(19) United States
(12) Patent Application Publication
(10) Pub. No.: US 2025/0059220 A1
(43) Pub. Date: Feb. 20, 2025

(54) SUBSTITUTED BENZOTHIOPHENE DERIVATIVES AND METHODS OF USE THEREOF

(71) Applicant: Merck Sharp & Dohme LLC, Rahway, NJ (US)

(72) Inventors: David A. Candito, Wrentham, MA (US); Matthew L. Childers, Medfield, MA (US); Duane E. DeMong, Hanover, MA (US); Christian Fischer, Natick, MA (US); Xavier Fradera, Jamaica Plain, MA (US); James P. Jewell, Newton, MA (US); Shuhei Kawamura, Cambridge, MA (US); Ping Liu, Westfield, NJ (US); Charles S. Yeung, Dedham, MA (US); Xiao Mei Zheng, Dover, MA (US); Kaitlyn Marie Logan, Jamaica Plain, MA (US); Ryan D. Otte, Natick, MA (US); Chunhui Huang, Arlington, MA (US); Sebastian E. Schneider, Cambridge, MA (US)

(73) Assignee: Merck Sharp & Dohme LLC, Rahway, NJ (US)

(21) Appl. No.: 18/720,698

(22) PCT Filed: Dec. 15, 2022
(86) PCT No.: PCT/US2022/052937

§ 371 Date: Jun. 17, 2024

Related U.S. Application Data
(60) Provisional application No. 63/293,167, filed on Dec. 23, 2021.

(51) Int. Cl. C07F 9/6553; A61K 31/67; A61K 31/675; A61K 31/695; A61K 45/06; C07F 9/6558; C07F 9/6561

(52) U.S. Cl. CPC C07F 9/655354; A61K 31/67; A61K 31/675; A61K 31/695; A61K 45/06; C07F 9/65586; C07F 9/6561

(57) ABSTRACT

Provided are novel Substituted Thiophene Derivatives of Formula (I) and pharmaceutically acceptable salts thereof, wherein R¹, R², R³, R⁴, and R⁷ are as defined herein, as well as compositions comprising at least one Substituted Thiophene Derivative, and methods of using the Substituted Thiophene Derivatives for treating or preventing cancer in a patient.

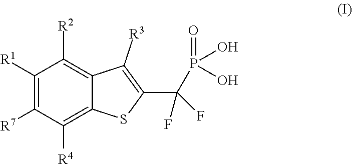

### FIELD OF THE INVENTION

The present disclosure relates to novel Substituted Benzothiophene Derivatives, compositions comprising at least one Substituted Benzothiophene Derivative, and methods of using the Substituted Benzothiophene Derivatives for treating or preventing a cellular proliferative disorder in a patient.

### BACKGROUND OF THE INVENTION

Cancer immunotherapy regimens targeting immune evasion mechanisms including checkpoint blockade (e.g., PD-1/PD-L1 and A CTLA-4 blocking antibodies) have been shown to be effective in treating in a variety of cancers, dramatically improving outcomes in some populations refractory to conventional therapies. However, incomplete clinical responses and the development of intrinsic or acquired resistance will continue to limit the patient populations who could benefit from checkpoint blockade.

Protein tyrosine phosphatase non-receptor type 2 (PTPN2), also known as T cell protein tyrosine phosphatase (TC-PTP), is an intracellular member of the class 1 subfamily of phospho-tyrosine specific phosphatases that control multiple cellular regulatory processes by removing phosphate groups from tyrosine substrates. PTPN2 is ubiquitously expressed, but expression is highest in hematopoietic and placental cells. In humans, PTPN2 expression is controlled post-transcriptionally by the existence of two splice variants: a 45 kDa form that contains a nuclear localization signal at the C-terminus upstream of the splice junction, and a 48 kDa canonical form which has a C-terminal ER retention motif. The 45 kDa isoform can passively transfuse into the cytosol under certain cellular stress conditions. Both isoforms share an N-terminal phospho-tyrosine phosphatase catalytic domain. PTPN2 negatively regulates signaling of non-receptor tyrosine kinases (e.g., JAK1, JAK3), receptor tyrosine kinases (e.g., INSR, EGFR, CSF1R, PDGFR), transcription factors (e.g. STAT1, STAT3, STAT5a/b), and Src family kinases (e.g., Fyn, Lck). As a critical negative regulator of the JAK-STAT pathway, PTPN2 functions to directly regulate signaling through cytokine receptors, including IFNγ. The PTPN2 catalytic domain shares 74% sequence homology with PTPN1, and shares similar enzymatic kinetics.

Data from a loss of function in vivo genetic screen using CRISPR/Cas9 genome editing in a mouse B16F10 transplantable tumor model show that deletion of PTPN2 gene in tumor cells improved response to the immunotherapy regimen of a GM-CSF secreting vaccine (GVAX) plus PD-1 checkpoint blockade. Loss of PTPN2 sensitized tumors to immunotherapy by enhancing IFNγ-mediated effects on antigen presentation and growth suppression. The same screen also revealed that genes known to be involved in immune evasion, including PD-L1 and CD47, were also depleted under immunotherapy selective pressure, while genes involved in the IFNγ signaling pathway, including IFNGR, JAK1, and STAT1, were enriched. These observations point to a putative role for therapeutic strategies that enhance IFNγ sensing and signaling in enhancing the efficacy of cancer immunotherapy regimens.

The prototypic tyrosine-specific phosphatase non-receptor type 1 (PTPN1), also known as protein tyrosine phosphatase-1B (PTP1B) is expressed ubiquitously and has been implicated in various physiological and pathological processes. Several studies suggest that PTPN1 can serve as a potential therapeutic target in solid tumors. In particular, PTPN1 levels are elevated in breast cancer where it is thought to contribute to tumor growth. Consistent with this, the global deletion of PTPN1 attenuates the development of mammary tumors driven by mutant ErbB2 in mice, whereas MSI-1436 attenuates the growth of xenografts in SCID-beige mice and the metastasis of ErbB2-driven mammary tumors in transgenic mice. These studies have focused on the cell autonomous contributions of PTPN1 in breast cancer tumorigenesis.

It is well-established that PTPN1 can attenuate JAK/STAT signaling by dephosphorylating and inactivating JAK-2 and Tyk2, and it has been shown that PTPN1 attenuates JAK/STAT-5 signaling and antagonises the expansion and activation of T cells. Also demonstrated is that PTPN1 abundance is increased in intratumoral CD8+ T cells to repress antitumor immunity. Using genetic approaches, it has been established that the deletion of PTPN1 in T cells promotes STAT-5 signaling to facilitate the antigen-induced expansion, activation and cytotoxicity of CD8+ T cells to attenuate the growth of solid tumors. It has been reported that the inhibition of PTPN1 in T cells enhances not only endogenous T cell-mediated antitumor immunity and the response to anti-PD-1 therapy, but also the efficacy of adoptively transferred T cells and CAR T cells to repress the growth of solid tumors. These findings define a novel intracellular checkpoint and actionable therapeutic target for enhancing the antitumor activity of T cells.

There remains a need in the art for compounds that can potentially treat cancer by inhibiting PTPN1 and/or PTPN2. The presently disclosed Substituted Benzothiophene Derivatives help address that need.

### SUMMARY OF THE INVENTION

In one aspect, provided are Compounds of Formula (I)

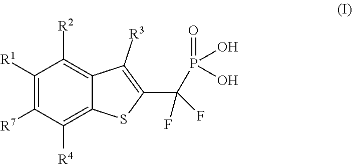

or a pharmaceutically acceptable salt thereof,
wherein:
R¹ is selected from H, C₁-C₁₀ alkyl, C₂-C₁₀ alkynyl, halo, —OH, —C(O)OH, —CN, —S(O)₂R⁹, —C(O)N(R⁵)(R⁶), —NHC(O)N(R⁵)(R⁶), —(C₁-C₁₀ alkenyl)ₙ-NHC(O)—(C₁-C₁₀ alkyl), —(C₁-C₁₀ alkenyl)ₙ-S(O)(NH)R⁹, NHC(O)—(C₁-C₁₀ alkenyl)ₙ-(5 or 6-membered monocyclic heteroaryl), —NH₂, —NHC(O)—CF₃, —(C₁-C₁₀ alkenyl)ₙ-NHC(O)—(C₃-C₇ monocyclic cycloalkyl), —O—(C₁-C₁₀ alkyl), —C(═NH)(NH₂), —O—(C₁-C₁₀ alkylene)-(C₆-C₁₀ aryl), —O—(C₆-C₁₀ aryl), —O-(5 or 6-membered monocyclic heteroaryl), —O-(8 to 10-membered bicyclic heteroaryl), 5 or 6-membered monocyclic heteroaryl, 4 to 6-membered monocyclic heterocycloalkenyl, —(8 to 10-membered bicyclic heteroaryl), —CH₂-(3 to 7-membered monocyclic heterocycloalkyl), 9 or 10-membered bicyclic heterocycloalkyl, —CH(NH₂)(CF₃), and —CH(NH)-phenyl, wherein said phenyl group, said C₆-C₁₀ aryl group, said C₃-C₇ monocyclic cycloalkyl group, said 5 or 6-membered monocyclic heteroaryl group, said 4 to 6-membered heterocycloalkenyl group, said 3 to 7-membered monocyclic heterocycloalkyl group, and said 9 or 10-membered monocyclic heterocycloalkyl group may be optionally substituted with 1-3 groups, which can be the same or different, and which are selected from C₁-C₁₀ alkyl, C₁-C₁₀ haloalkyl, —O—(C₁-C₁₀ alkyl), C₃-C₇ monocyclic cycloalkyl, and halo; and wherein said C₃-C₇ monocyclic cycloalkyl group, said 4 to 6-membered heterocycloalkenyl group, and said 3 to 7-membered monocyclic heterocycloalkyl group can have one or more ring carbon atoms or ring heteroatoms optionally substituted with an oxo group; and wherein any C₁-C₁₀ alkyl group can be optionally substituted with 1-3 groups, which can be the same or different, and which are selected from —OH, halo, —CN, —OR⁹, —N(R⁹)₂, —SO₂(R⁹), —S(O)₂N(R⁹)₂, —S(O)NH(R⁹), halo, —NHC(O)R⁹, and —C(O)N(R⁹)₂;
R² is H or halo;
R³ is selected from H, halo, —CN, C₁-C₁₀ alkyl, C₃-C₇ monocyclic cycloalkyl, and C₁-C₁₀ haloalkyl;
R⁴ is selected from H, halo, —OH, —CN, —(C₁-C₁₀ alkylene)ₙ-(5 or 6-membered monocyclic heteroaryl), —(C₁-C₁₀ alkylene)-(C₆-C₁₀ aryl), —(C₁-C₁₀ alkylene)-S-(5 or 6-membered monocyclic heteroaryl), —(C₁-C₁₀ alkylene)-Si(C₁-C₆ alkyl)₃, —O—(C₁-C₁₀ alkyl), —S—(C₁-C₁₀ alkyl), —O—(C₁-C₁₀ alkenylene), —O—(C₁-C₁₀ haloalkyl), —O—(C₁-C₁₀ hydroxyalkyl), —O—(C₁-C₁₀ alkylene)-S(O)₂R⁹, —O—(C₁-C₁₀ alkylene)-CN, —O—(C₁-C₁₀ alkylene)-O—(C₁-C₁₀ alkyl), —O—(C₁-C₁₀ alkylene)-O—(C₁-C₁₀ haloalkyl), —O—(C₁-C₁₀ alkylene)-(C₆-C₁₀ aryl), —O—(C₁-C₁₀ alkylene)-(C₃-C₇ monocyclic cycloalkyl), —O—(C₁-C₁₀ alkylene)-(3 to 7-membered monocyclic heterocycloalkyl), O—(C₁-C₁₀ alkylene)-(5 to 10-membered bridged heterocycloalkyl), —O—(C₁-C₁₀ alkylene)-(5 or 6-membered monocyclic heteroaryl), —C₁-C₁₀ alkenyl, —(C₁-C₁₀ alkylene)-O—(C₁-C₁₀ alkyl), —O—(C₁-C₁₀ alkylene)-S(O)₂N(R⁹)₂, —O—(C₁-C₁₀ alkylene)-NHS(O)₂—(C₁-C₁₀ alkyl), —O—(C₁-C₁₀ alkylene)-NHS(O)₂—(C₁-C₁₀ haloalkyl), —O—(C₁-C₁₀ alkylene)-NHS(O)₂—(C₃-C₇ monocyclic cycloalkyl), —O—(C₁-C₁₀ alkylene)-(C₅-C₁₀ bicyclic cycloalkyl), —O—(C₁-C₁₀ alkylene)-C(O)NH-phenyl, —O—(C₁-C₁₀ alkylene)-C(O)NH₂, —O—(C₁-C₁₀ alkylene)-C(O)NH-(5 or 6-membered monocyclic heteroaryl), —O—(C₁-C₁₀ alkylene)-C(O)NH—(C₃-C₇ monocyclic cycloalkyl), —O—(C₁-C₁₀ alkylene)-C(O)NH—(C₁-C₁₀ alkyl), —O—(C₁-C₁₀ alkylene)-C(O)NH—(C₁-C₁₀ haloalkyl), —(C₁-C₁₀ alkylene)ₙ-(3 to 7-membered monocyclic heterocycloalkyl), —(C₁-C₁₀ alkylene)ₙ-(8 to 10-membered bicyclic heteroaryl), —(C₁-C₁₀ alkylene)ₙ-(9 to 14-membered tricyclic heteroaryl), —(C₁-C₁₀ alkylene)ₙ-(10 to 14-membered tricyclic heterocycloalkyl), —O—(C₁-C₁₀ alkylene)ₙ-(3 to 7-membered monocyclic heterocycloalkyl), —O—(C₁-C₁₀ alkylene)ₙ-(C₅-C₁₀ membered bicyclic cycloalkyl), —O—(C₁-C₁₀ alkylene)ₙ-(10 to 14-membered tricyclic heterocycloalkyl), —NH—(C₁-C₁₀ alkyl), —NH—(C₁-C₁₀ haloalkyl), —NH—(C₁-C₁₀ hydroxyalkyl), —NH—(C₁-C₁₀ alkylene)-CN, —NH—(C₁-C₁₀ alkylene)-O—(C₁-C₁₀ alkyl), —NH—(C₁-C₁₀ alkylene)-O—(C₁-C₁₀ haloalkyl), —NH—(C₁-C₁₀ alkylene)-(C₆-C₁₀ aryl), —NH—(C₁-C₁₀ alkylene)-(C₃-C₇ monocyclic cycloalkyl), —NH—(C₁-C₁₀ alkylene)-(3 to 7-membered monocyclic heterocycloalkyl), —NH—(C₁-C₁₀ alkylene)-(5 or 6-membered monocyclic heteroaryl), —NH—(C₁-C₁₀ haloalkyl), —NH—(C₁-C₁₀ hydroxyalkyl), —NH—(C₁-C₁₀ alkylene)-S(O)₂—(C₁-C₁₀ alkyl), —NH—(C₁-C₁₀ alkylene)-S(O)₂N(R⁹)₂, —NH—(C₁-C₁₀ alkylene)-NHS(O)₂—(C₁-C₁₀ alkyl), —NH—(C₁-C₁₀ alkylene)-NHS(O)₂—(C₁-C₁₀ haloalkyl), —NH—(C₁-C₁₀ alkylene)-NHS(O)₂—(C₃-C₇ monocyclic cycloalkyl), —NH—(C₁-C₁₀ alkylene)-(C₆-C₁₀ bicyclic cycloalkyl), —NH—(C₁-C₁₀ alkylene)-C(O)NH-phenyl, —NH—(C₁-C₁₀ alkylene)-C(O)NH—(C₃-C₇ monocyclic cycloalkyl), —NH—(C₁-C₁₀ alkylene)-C(O)NH—(C₁-C₁₀ alkyl), —NH—(C₁-C₁₀ alkylene)-C(O)NH—(C₁-C₁₀ haloalkyl), —NH—(C₁-C₁₀ alkylene)ₙ-(3 to 7-membered monocyclic heterocycloalkyl), —NH—(C₁-C₁₀ alkylene)ₙ-(6 to 10 membered bicyclic cycloalkyl), —(C₁-C₁₀ alkylene)ₙ-S(O)₂N(R⁹)₂, and —NH—(C₁-C₁₀ alkylene)ₙ-(10 to 14-membered tricyclic heterocycloalkyl), wherein said 5 or 6-membered monocyclic heteroaryl group, said 3 to 7-membered monocyclic heterocycloalkyl group, said C₆-C₁₀ aryl group, said 8 to 10-membered bicyclic heteroaryl group, said 6 to 10-membered bicyclic heterocycloalkyl group, said 10 to 14-membered tricyclic heterocycloalkyl group, said C₃-C₇ monocyclic cycloalkyl group, said 5 to 10 membered bridged cycloalkyl group, 5 to 10-membered bridged heterocycloalkyl group and C₆-C₁₀ bicyclic cycloalkyl group may be optionally substituted with one or more R^{A} groups, which can be the same or different; and wherein the C₁-C₁₀ alkyl moiety of a —O—(C₁-C₁₀ alkyl) group can be optionally substituted with 1-3 groups, which can be the same or different, and which are selected from —OH, halo, —CN, —OR⁹, —N(R⁹)₂, —SO₂(R⁹), —S(O)₂N(R⁹)₂, —S(O)NH(R⁹), halo, —NHC(O)R⁹, and —C(O)N(R⁹)₂;
R⁵ is selected from H, C₁-C₁₀ alkyl, and —(C₁-C₁₀ alkylene)-O—(C₁-C₁₀ alkyl);
R⁶ is selected from H, C₁-C₁₀ alkyl, —OR⁹, —(C₁-C₁₀ alkylene)-S—(C₁-C₁₀ haloalkyl), —(C₁-C₁₀ alkylene)ₙ-OR⁹, —(C₁-C₁₀ alkylene)ₙ-(C₆-C₁₀ aryl), —(C₁-C₁₀ alkylene)ₙ-CN, —(C₁-C₁₀ alkylene)ₙ-(5 or 6-membered monocyclic heteroaryl), —(C₁-C₁₀ alkylene)ₙ-(8 to 10-membered bicyclic heteroaryl), —(C₁-C₁₀ alkylene)ₙ-(3 to 7-membered monocyclic cycloalkyl), —(C₁-C₁₀ alkylene)ₙ-(3 to 7-membered monocyclic heterocycloalkyl), —(C₁-C₁₀ alkylene)ₙ-(6 to 10 membered bicyclic heterocycloalkyl), and —(C₁-C₁₀ alkylene)ₙ-(10 to 14-membered membered tricyclic heterocycloalkyl) group, wherein said C₆-C₁₀ aryl group, said 5 or 6-membered monocyclic heteroaryl group, said 8 to 10-membered bicyclic heteroaryl group, said 10 to 14-membered membered tricyclic heterocycloalkyl group, said C₃-C₇ monocyclic cycloalkyl group, said 3 to 7-membered monocyclic heterocycloalkyl group, and said 6 to 10 membered bicyclic heterocycloalkyl group can each be optionally substituted with one or more R groups, which can be the same or different, and wherein the C₁-C₁₀ alkyl group or C₁-C₁₀ alkylenyl group can be optionally substituted with 1-3 groups, which can be the same or different, and which are selected from —OH, halo, —CN, —OR⁹, —N(R⁹)₂, —SO₂(R⁹), —S(O)₂N(R⁹)₂, —S(O)NH(R⁹), halo, phenyl, —NHC(O)R⁹, C₁-C₁₀ haloalkyl, and —C(O)N(R⁹)₂;
R⁷ is selected from H, halo, CN, —C(O)N(R⁸)₂, —O—(C₁-C₁₀ hydroxyalkyl), and —NHC(O)R⁸;
each occurrence of R⁸ is independently selected from H, C₁-C₁₀ alkyl, phenyl, and 5 or 6-membered monocyclic heteroaryl;
each occurrence of R⁹ is independently selected from H, C₁-C₁₀ alkyl, C₁-C₁₀ haloalkyl, —SO₂CH₃, C₃-C₇ monocyclic cycloalkyl, 3 to 7-membered monocyclic heterocycloalkyl, C₆-C₁₀ aryl, —(C₁-C₁₀ alkylene)ₙ-(C₆-C₁₀ aryl), and 5 or 6-membered monocyclic heteroaryl;
each occurrence of R^{A} is independently selected from C₁-C₁₀ alkyl, halo, —CN, C₁-C₁₀ haloalkyl, —O—(C₁-C₁₀ haloalkyl), oxo, —O—(C₁-C₁₀ alkyl), —C₃-C₇ monocyclic cycloalkyl, 5 or 6-membered monocyclic heteroaryl, C₆-C₁₀ aryl, —C(O)-(3 to 7-membered monocyclic heterocycloalkyl), —S(O)₂—(C₁-C₁₀ alkyl), and —O-(3 to 7-membered monocyclic heterocycloalkyl); wherein said C₃-C₇ monocyclic cycloalkyl group and said 3 to 7-membered monocyclic heterocycloalkyl group can be further substituted with C₁-C₁₀ alkyl, halo, C₁-C₁₀ haloalkyl or —CN;
each occurrence of R^{B} is independently selected from C₁-C₁₀ alkyl, halo, —OH, oxo, —SO2NH2, C₁-C₁₀ haloalkyl, C₁-C₁₀ hydroxyalkyl, —O—(C₁-C₁₀ alkyl), —O—(C₁-C₁₀ alkylene)-O—(C₁-C₁₀ alkyl), —O—(C₁-C₁₀ haloalkyl), —CN, —C(O)—(C₁-C₁₀ alkyl), —C(O)—(C₃-C₇ monocyclic cycloalkyl), —C(O)-(3 to 7-membered monocyclic heterocycloalkyl), —O-(3 to 7-membered monocyclic heterocycloalkyl), —S(O)₂—(C₁-C₁₀ alkyl), phenyl, benzyl, —O-phenyl, —O-benzyl, —S— phenyl, C₃-C₇ monocyclic cycloalkyl, —O—(C₁-C₁₀ alkylene)-phenyl, —O—(C₁-C₁₀ alkylene)ₙ-(3 to 7-membered monocyclic heterocycloalkyl), —(C₁-C₁₀ alkylene)ₙ-(5 or 6-membered monocyclic heteroaryl); wherein said phenyl group, said C₃-C₇ monocyclic cycloalkyl group, said 3 to 7-membered monocyclic heterocycloalkyl group, and said 5 or 6-membered monocyclic heteroaryl group, can be optionally substituted with 1-3 groups, which can be the same or different, and are selected from C₁-C₁₀ alkyl, —O—(C₁-C₁₀ alkyl), halo, C₁-C₁₀ haloalkyl, CN, and —OH; and
each occurrence of n is independently 0 or 1.

The Compounds of Formula (I) (also referred to herein as the “Substituted Benzothiophene Derivatives”), and pharmaceutically acceptable salts thereof, can be useful for treating or preventing a cellular proliferative disorder in a patient. Without being bound by any specific theory, it is believed that the Substituted Benzothiophene Derivatives act as inhibitors of protein tyrosine phosphatases (e.g, PTPN1 and/or PTPN2).

Accordingly, provided herein are methods for treating or preventing a cellular proliferative disorder in a patient, comprising administering to the patient an effective amount of at least one Substituted Benzothiophene Derivative.

Further details are set forth in the accompanying detailed description below.

Although any methods and materials similar to those described herein can be used in the practice or testing of the Substituted Benzothiophene Derivatives, illustrative methods and materials are now described. Other embodiments, aspects and features are either further described in or will be apparent from the ensuing description, examples and appended claims.

### DETAILED DESCRIPTION OF THE INVENTION

Described are novel Substituted Benzothiophene Derivatives, compositions comprising at least one Substituted Benzothiophene Derivative, and methods of using the Substituted Benzothiophene Derivatives for treating or preventing a cellular proliferative disorder in a patient.

### Definitions and Abbreviations

The terms used herein have their ordinary meaning and the meaning of such terms is independent at each occurrence thereof. That notwithstanding and except where stated otherwise, the following definitions apply throughout the specification and claims. Chemical names, common names, and chemical structures may be used interchangeably to describe the same structure. If a chemical compound is referred to using both a chemical structure and a chemical name and an ambiguity exists between the structure and the name, it is to be understood that the structure predominates. These definitions apply regardless of whether a term is used by itself or in combination with other terms, unless otherwise indicated. Hence, the definition of “alkyl” applies to “alkyl” as well as the “alkyl” portions of “hydroxyalkyl,” “haloalkyl,” “—O-alkyl,” etc. . . .

As used herein, and throughout this disclosure, the following terms, unless otherwise indicated, shall be understood to have the following meanings:

A “patient” is a human or non-human mammal. In one embodiment, a patient is a human.

The term “effective amount” as used herein, refers to an amount of Substituted Benzothiophene Derivative, and/or an additional therapeutic agent, or a composition thereof that is effective in producing the desired therapeutic, ameliorative, inhibitory or preventative effect when administered to a patient suffering from a cellular proliferative disorder. In the combination therapies described herein, an effective amount can refer to each individual agent or to the combination as a whole, wherein the amounts of all agents administered are together effective, but wherein the component agent of the combination may not be present individually in an effective amount.

The term “preventing,” as used herein with respect to a cellular proliferative disorder, refers to reducing the likelihood of a cellular proliferative disorder.

The term “alkyl,” as used herein, refers to an aliphatic hydrocarbon group having one of its hydrogen atoms replaced with a bond. An alkyl group may be straight or branched and contain from about 1 to about 20 carbon atoms. In one embodiment, an alkyl group contains from about 1 to about 10 carbon atoms. In different embodiments, an alkyl group contains from 1 to 10 carbon atoms (C₁-C₁₀ alkyl) or from about 1 to about 6 carbon atoms (C₁-C₆ alkyl). Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl and neohexyl. An alkyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, NH(cycloalkyl), —O—C(O)-alkyl, —O—C(O)-aryl, —O—C(O)-cycloalkyl, —C(O)OH and —C(O)O-alkyl. In one embodiment, an alkyl group is linear. In another embodiment, an alkyl group is branched. Unless otherwise indicated, an alkyl group is unsubstituted.

The term “alkenyl,” as used herein, refers to an aliphatic hydrocarbon group containing at least one carbon-carbon double bond and having one of its hydrogen atoms replaced with a bond. An alkenyl group may be straight or branched and contain from about 2 to about 15 carbon atoms. In one embodiment, an alkenyl group contains from about 2 to about 10 carbon atoms. In another embodiment, an alkenyl group contains from about 2 to about 6 carbon atoms. Non-limiting examples of alkenyl groups include ethenyl, propenyl, n-butenyl, 3-methylbut-2-enyl, n-pentenyl, octenyl and decenyl. An alkenyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. The term “C₂-C₁₀ alkenyl” refers to an alkenyl group having from 2 to 10 carbon atoms. Unless otherwise indicated, an alkenyl group is unsubstituted.

The term “alkynyl,” as used herein, refers to an aliphatic hydrocarbon group containing at least one carbon-carbon triple bond and having one of its hydrogen atoms replaced with a bond. An alkynyl group may be straight or branched and contain from about 2 to about 15 carbon atoms. In one embodiment, an alkynyl group contains from about 2 to about 10 carbon atoms. In another embodiment, an alkynyl group contains from about 2 to about 6 carbon atoms. Non-limiting examples of alkynyl groups include ethynyl, propynyl, 2-butynyl and 3-methylbutynyl. An alkynyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. The term “C₂-C₁₀ alkynyl” refers to an alkynyl group having from 2 to 10 carbon atoms. Unless otherwise indicated, an alkynyl group is unsubstituted.

The term “alkylene,” as used herein, refers to an alkyl group, as defined above, wherein one of the alkyl group's hydrogen atoms has been replaced with a bond. Non-limiting examples of alkylene groups include —CH₂—, —CH₂CH₂—, —CH₂CH₂CH₂—, —CH₂CH₂CH₂CH₂—, —CH(CH₃)CH₂CH₂—, —CH(CH₃)— and —CH₂CH(CH₃)CH₂—. In one embodiment, an alkylene group has from 1 to about 10 carbon atoms. In another embodiment, an alkylene group has from 1 to about 6 carbon atoms. In another embodiment, an alkylene group is branched. In another embodiment, an alkylene group is linear. In one embodiment, an alkylene group is —CH₂—. The term “C₁-C₁₀ alkylene” refers to an alkylene group having from 1 to 10 carbon atoms.

The term “alkenylene,” as used herein, refers to an alkenyl group, as defined above, wherein one of the alkenyl group's hydrogen atoms has been replaced with a bond. Non-limiting examples of alkylene groups include —CH═CH—, —CH═CHCH₂—, —CH₂CH₂CH═CH—, and —CH₂(CH₃)C═CH—. In one embodiment, an alkenylene group has from 1 to about 6 carbon atoms.

In another embodiment, an alkenylene group is branched. In another embodiment, an alkenylene group is linear. The term “C₁-C₆ alkenylene” refers to an alkenylene group having from 1 to 6 carbon atoms.

The term “alkynylene,” as used herein, refers to an alkynyl group, as defined above, wherein one of the alkynyl group's hydrogen atoms has been replaced with a bond. Non-limiting examples of alkylene groups include —C≡C—, —C≡CCH₂—, and —C≡CCH(CH₃)₂—. In one embodiment, an alkynylene group has from 1 to about 10 carbon atoms. In another embodiment, an alkynylene group has from 1 to about 6 carbon atoms. In another embodiment, an alkynylene group is branched. In another embodiment, an alkynylene group is linear. The term “C₁-C₁₀ alkynylene” refers to an alkynylene group having from 1 to 10 carbon atoms. The term “C₁-C₆ alkynylene” refers to an alkynylene group having from 1 to 6 carbon atoms.

The term “aminoalkyl,” as used herein, refers to an alkyl group as defined above, wherein one of the alkyl group's hydrogen atoms has been replaced with —NH₂, —NH(C₁-C₆ alkyl), or —N(C₁-C₆ alkyl)₂. In one embodiment, an aminoalkyl group has from 1 to 6 carbon atoms. Non-limiting examples of aminoalkyl groups include —CH₂NH₂, —CH₂N(CH₃)₂, —CH₂NH₂, and —CH₂NH(CH)₃. The term “C₁-C₆ aminoalkyl” refers to an aminoalkyl group having from 1 to 6 carbon atoms.

The term “aryl,” as used herein, refers to an aromatic monocyclic or multicyclic ring system comprising from about 6 to about 14 carbon. In one embodiment, an aryl group contains from about 6 to about 10 carbon atoms (“C₆-C₁₀ aryl” group). An aryl group can be optionally substituted with one or more “ring system substituents” which may be the same or different, and are as defined herein below. In one embodiment, an aryl group can be optionally fused to a cycloalkyl or cycloalkanoyl group. Non-limiting examples of aryl groups include phenyl and naphthyl. An example of an aryl group fused to a cycloalkyl ring includes:

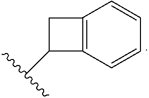 [SMILES: *C1Cc2ccccc21]

In one embodiment, an aryl group is phenyl. In another embodiment, an aryl group is napthalene. Unless otherwise indicated, an alkyl group is unsubstituted.

The term “cycloalkyl,” as used herein, refers to a non-aromatic mono- or multicyclic ring system comprising from about 3 to about 10 ring carbon atoms. In one embodiment, a cycloalkyl contains from about 5 to about 10 ring carbon atoms. In another embodiment, a cycloalkyl is monocyclic, and contains from about 3 to about 7 ring atoms. In another embodiment, a cycloalkyl is monocyclic, and contains from about 5 to about 6 ring atoms. In another embodiment, a cycloalkyl is bicyclic and contains about 4 to 10 ring atoms. Non-limiting examples of monocyclic cycloalkyls include cyclopropyl, cyclobutyl, cyclopentyl, cyclohexyl, cycloheptyl and cyclooctyl. Non-limiting examples of multicyclic cycloalkyls include 1-decalinyl, norbornyl and adamantyl. A cycloalkyl group can be optionally substituted with one or more “ring system substituents” which may be the same or different, and are as defined herein below. Unless otherwise indicated, cycloalkyl group is unsubstituted. In one embodiment, a cycloalkyl group is unsubstituted. The term “3 to 6-membered monocyclic cycloalkyl” refers to a monocyclic cycloalkyl group having from 3 to 6 ring carbon atoms. The term “3 to 7-membered monocyclic cycloalkyl” refers to a monocyclic cycloalkyl group having from 3 to 7 ring carbon atoms. The term “4 to 10-membered bicyclic cycloalkyl group” refers to a bicyclic cycloalkyl group having from 4 to 10 ring carbon atoms.

A multicyclic cycloalkyl group may have rings that are fused, rings that are joined in a spirocyclic manner, and rings that are bridged. In one embodiment, a cycloalkyl group can be a bicyclic spirocyclic cycloalkyl group having from 6 to 10 ring carbon atoms. Illustrative examples of such a bicyclic cycloalkyl group include:

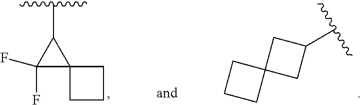 [SMILES: *C1C(F)(F)C12CCC2 ; *C1CC2(CCC2)C1]

In another embodiment, a cycloalkyl group can be a bicyclic fused cycloalkyl group having from 6 to 10 ring carbon atoms. Illustrative examples of such a fused bicyclic cycloalkyl group include:

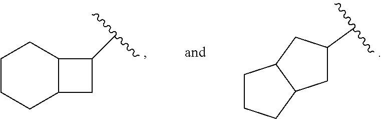 [SMILES: *C1CC2CCCCC12 ; *C1CC2CCCC2C1]

In another embodiment, a cycloalkyl group can be a bridged bicyclic cycloalkyl group having from 5 to 10 ring carbon atoms, or a bridged tricyclic cycloalkyl group having from 6 to 14 ring carbon atoms. Illustrative examples of such bridged bicyclic and tricyclic heterocycloalkyl groups include:

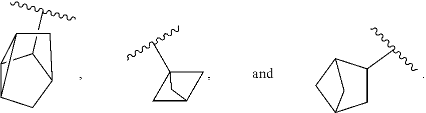 [SMILES: *C1C2CC3C(C2)C13 ; *C12CC(C1)C2 ; *C1CC2CC1C2]

A ring carbon atom of a cycloalkyl group may be functionalized as a carbonyl group. An illustrative example of such a cycloalkyl group (also referred to herein as a “cycloalkanoyl” group) includes, but is not limited to, cyclobutanoyl:

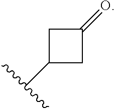 [SMILES: *C1CC(=O)C1]

The term “cycloalkenyl,” as used herein, refers to a non-aromatic mono- or multicyclic ring system comprising from about 4 to about 10 ring carbon atoms and containing at least one endocyclic double bond. In one embodiment, a cycloalkenyl contains from about 4 to about 7 ring carbon atoms. In another embodiment, a cycloalkenyl contains 5 or 6 ring atoms. Non-limiting examples of monocyclic cycloalkenyls include cyclopentenyl, cyclohexenyl, cyclohepta-1,3-dienyl, and the like. A cycloalkenyl group can be optionally substituted with one or more “ring system substituents” which may be the same or different, and are as defined herein below. A ring carbon atom of a cycloalkyl group may be functionalized as a carbonyl group. In one embodiment, a cycloalkenyl group is cyclopentenyl. In another embodiment, a cycloalkenyl group is cyclohexenyl. The term “4 to 6-membered cycloalkenyl” refers to a cycloalkenyl group having from 4 to 6 ring carbon atoms.

The term “halo,” as used herein, means —F, —Cl, —Br or —I.

The term “haloalkyl,” as used herein, refers to an alkyl group as defined above, wherein one or more of the alkyl group's hydrogen atoms has been replaced with a halogen. In one embodiment, a haloalkyl group has from 1 to 10 carbon atoms. In another embodiment, a haloalkyl group has from 1 to 6 carbon atoms. In another embodiment, a haloalkyl group is substituted with from 1 to 6 F atoms. In a class of this embodiment, the haloalkyl group is substituted with from 1 to 3 F atoms. Non-limiting examples of haloalkyl groups include —CH₂CHF₂, —CH₂F, —CHF₂, —CF₃, —CH₂Cl and —CCl₃. The term “C₁-C₁₀ haloalkyl” refers to a haloalkyl group having from 1 to 10 carbon atoms.

The term “hydroxyalkyl,” as used herein, refers to an alkyl group as defined above, wherein one or more of the alkyl group's hydrogen atoms has been replaced with an —OH group. In one embodiment, a hydroxyalkyl group has from 1 to 10 carbon atoms. In another embodiment, a hydroxyalkyl group has from 1 to 6 carbon atoms. Non-limiting examples of hydroxyalkyl groups include —CH₂OH, —CH₂CH₂OH, —CH₂CH₂CH₂OH and —CH₂CH(OH)CH₃. The term “C₁-C₁₀ hydroxyalkyl” refers to a hydroxyalkyl group having from 1 to 10 carbon atoms.

The term “heteroaryl,” as used herein, refers to an aromatic monocyclic or multicyclic ring system comprising about 5 to about 14 ring atoms, wherein from 1 to 4 of the ring atoms is independently O, N or S and the remaining ring atoms are carbon atoms. In one embodiment, a heteroaryl group has 5 to 10 ring atoms. In another embodiment, a heteroaryl group is monocyclic and has 5 or 6 ring atoms (“5 or 6-membered monocyclic heteroaryl”). In another embodiment, a heteroaryl group is bicyclic and had 8 to 10 ring atoms (“8 to 10-membered bicyclic heteroaryl”). In still another embodiment, a heteroaryl group is bicyclic and has 9 or 10 ring atoms (“9 or 10-membered bicyclic heteroaryl”). A heteroaryl group can be optionally substituted by one or more “ring system substituents” which may be the same or different, and are as defined herein below. A heteroaryl group is joined via a ring carbon atom, and any nitrogen atom of a heteroaryl can be optionally oxidized to the corresponding N-oxide. The term “heteroaryl” also encompasses a heteroaryl group, as defined above, which is fused to a benzene ring. Non-limiting examples of heteroaryls include pyridyl, pyrazinyl, furanyl, thienyl, pyrimidinyl, pyridone (including N-substituted pyridones), isoxazolyl, isothiazolyl, oxazolyl, oxadiazolyl, thiazolyl, pyrazolyl, furazanyl, pyrrolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, benzimidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like, and all isomeric forms thereof. The term “heteroaryl” also refers to partially saturated heteroaryl moieties such as, for example, tetrahydroisoquinolyl, tetrahydroquinolyl and the like. In one embodiment, a heteroaryl group is a 5-membered heteroaryl. In another embodiment, a heteroaryl group is a 6-membered heteroaryl, such as pyridyl.

In one embodiment, an 8 to 10-membered bicyclic heteroaryl group comprises a fused bicyclic heterocyclic group in which one of the two fused rings is phenyl or monocyclic heteroaryl, such as:

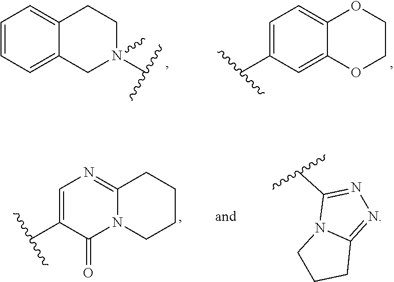

A “9 to 14-membered tricyclic heteroaryl” comprises an 8 to 10-membered bicyclic heteroaryl group, wherein a third ring is fused to one of the rings of the 8 to 10-membered bicyclic heteroaryl group. Such third ring can be a cycloalkyl, heterocycloalkyl, or heteroaryl ring. Examples of a 9 to 14-membered tricyclic heteroaryl group include:

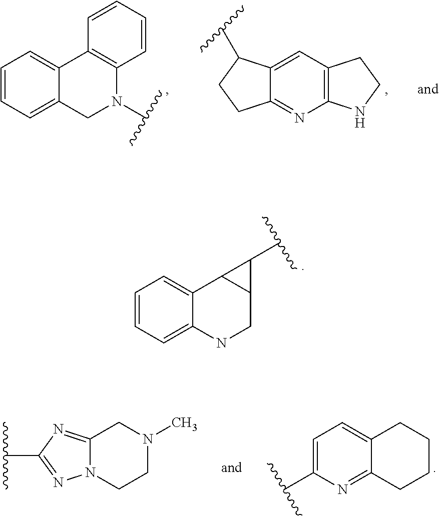 [SMILES: *N1Cc2ccccc2-c2ccccc21 ; *C1CCc2nc3c(cc21)CCN3 ; *C1C2CNc3ccccc3C12 ; *c1nc2n(n1)CCN(C)C2 ; *c1ccc2c(n1)CCCC2]

The term “heteroarylene,” as used herein, refers to a bivalent group derived from an heteroaryl group, as defined above, by removal of a hydrogen atom from a ring carbon or ring heteroatom of a heteroaryl group. A heteroarylene group can be derived from a monocyclic or multicyclic ring system comprising about 5 to about 14 ring atoms, wherein from 1 to 4 of the ring atoms are each independently O, N or S and the remaining ring atoms are carbon atoms. A heteroarylene group can be optionally substituted by one or more “ring system substituents” which may be the same or different, and are as defined herein below. A heteroarylene group is joined via a ring carbon atom or by a nitrogen atom with an open valence, and any nitrogen atom of a heteroarylene can be optionally oxidized to the corresponding N-oxide. The term “heteroarylene” also encompasses a heteroarylene group, as defined above, which is fused to a benzene ring. Non-limiting examples of heteroarylenes include pyridylene, pyrazinylene, furanylene, thienylene, pyrimidinylene, pyridonylene (including those derived from N-substituted pyridonyls), isoxazolylene, isothiazolylene, oxazolylene, oxadiazolylene, thiazolylene, pyrazolylene, thiophenylene, furazanylene, pyrrolylene, triazolylene, 1,2,4-thiadiazolylene, pyrazinylene, pyridazinylene, quinoxalinylene, phthalazinylene, oxindolylene, imidazo[1,2-a]pyridinylene, imidazo[2,1-b]thiazolylene, benzofurazanylene, indolylene, azaindolylene, benzimidazolylene, benzothienylene, quinolinylene, imidazolylene, benzimidazolylene, thienopyridylene, quinazolinylene, thienopyrimidylene, pyrrolopyridylene, imidazopyridylene, isoquinolinylene, benzoazaindolylene, 1,2,4-triazinylene, benzothiazolylene and the like, and all isomeric forms thereof. The term “heteroarylene” also refers to partially saturated heteroarylene moieties such as, for example, tetrahydroisoquinolylene, tetrahydroquinolylene, and the like. A heteroarylene group is divalent and unless specified otherwise, either available bond on a heteroarylene ring can connect to either group flanking the heteroarylene group. For example, the group “A-heteroarylene-B,” wherein the heteroarylene group is:

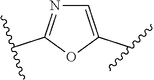 [SMILES: *c1cnc(*)o1]

is understood to represent both:

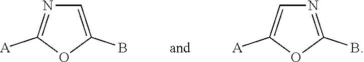

In one embodiment, a heteroarylene group is a monocyclic heteroarylene group or a bicyclic heteroarylene group. In another embodiment, a heteroarylene group is a monocyclic heteroarylene group. In another embodiment, a heteroarylene group is a bicyclic heteroarylene group. In still another embodiment, a heteroarylene group has from about 5 to about 10 ring atoms. In another embodiment, a heteroarylene group is monocyclic and has 5 or 6 ring atoms. In another embodiment, a heteroarylene group is bicyclic and has 9 or 10 ring atoms. In another embodiment, a heteroarylene group is a 5-membered monocyclic heteroarylene. In another embodiment, a heteroarylene group is a 6-membered monocyclic heteroarylene. In another embodiment, a bicyclic heteroarylene group comprises a 5 or 6-membered monocyclic heteroarylene group fused to a benzene ring. In still another embodiment, a heteroaryl group comprises a 5- to 6-membered monocyclic heteroarylene group fused to a cycloalkyl ring or a heterocycloalkyl ring.

The term “heterocycloalkyl,” as used herein, refers to a non-aromatic saturated monocyclic or multicyclic ring system comprising 3 to about 14 ring atoms, wherein from 1 to 4 of the ring atoms are independently O, S, N or Si, and the remainder of the ring atoms are carbon atoms. A heterocycloalkyl group can be joined via a ring carbon, ring silicon atom or ring nitrogen atom. In one embodiment, a heterocycloalkyl group is monocyclic. In one embodiment, a heterocycloalkyl group is monocyclic and has from about 3 to about 7 ring atoms (“3 to 7-membered monocyclic heterocycloalkyl”). In another embodiment, a heterocycloalkyl group is monocyclic has from about 4 to about 7 ring atoms (“4 to 7-membered monocyclic heterocycloalkyl”). In still another embodiment, a heterocycloalkyl group is monocyclic and has 5 or 6 ring atoms (“5 or 6-membered monocyclic heterocycloalkyl”). In one embodiment, a heterocycloalkyl group is bicyclic. In another embodiment, a heterocycloalkyl group is bicyclic and has from about 6 to about 10 ring atoms (“6 to 10-membered bicyclic heterocycloalkyl”). In another embodiment, a heterocycloalkyl group is tricyclic and has from about 10 to about 14 ring atoms (“10 to 14-membered tricyclic heterocycloalkyl”). There are no adjacent oxygen and/or sulfur atoms present in the ring system. Any —NH group in a heterocycloalkyl ring may exist protected such as, for example, as an —N(BOC), —N(CBz), —N(Tos) group and the like; such protected heterocycloalkyl groups are considered part of this disclosure. A heterocycloalkyl group can be optionally substituted by one or more “ring system substituents” which may be the same or different, and are as defined herein below. The nitrogen or sulfur atom of the heterocycloalkyl can be optionally oxidized to the corresponding N-oxide, S-oxide or S,S-dioxide. Non-limiting examples of monocyclic heterocycloalkyl rings include oxetanyl, piperidyl, pyrrolidinyl, piperazinyl, morpholinyl, thiomorpholinyl, thiazolidinyl, 1,4-dioxanyl, tetrahydrofuranyl, tetrahydrothiophenyl, delta-lactam, delta-lactone, silacyclopentane, silapyrrolidine and the like, and all isomers thereof. Non-limiting illustrative examples of a silyl-containing heterocycloalkyl group include:

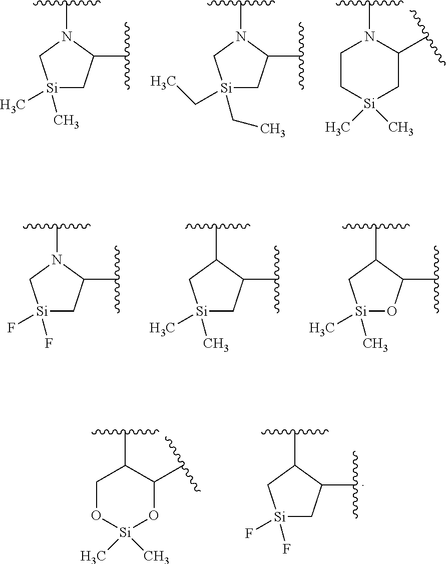

A ring carbon atom of a heterocycloalkyl group may be functionalized as a carbonyl group. Illustrative examples of such a heterocycloalkyl group include, but are not limited to:

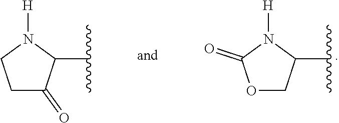 [SMILES: *C1NCCC1=O ; *C1COC(=O)N1]

A ring sulfur atom of a heterocycloalkyl group may also be functionalized as a sulfonyl group. An example of such a heterocycloalkyl group is:

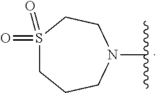 [SMILES: *N1CCCS(=O)(=O)CC1]

In one embodiment, a heterocycloalkyl group is a 5-membered monocyclic heterocycloalkyl. In another embodiment, a heterocycloalkyl group is a 6-membered monocyclic heterocycloalkyl.

A multicyclic heterocycloalkyl group may have rings that are fused, rings that are joined in a spirocyclic manner, and rings that are bridged. In one embodiment, a heterocycloalkyl group can be a bicyclic spirocyclic heteroaryl group having from 7 to 9 ring atoms. Illustrative examples of such a bicyclic heterocycloalkyl group include:

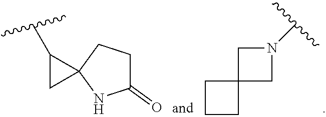 [SMILES: *C1CC12CCC(=O)N2 ; "and" ; *N1CC2(CCC2)C1]

In another embodiment, a heterocycloalkyl group can be a bicyclic fused heterocycloalkyl group having from 6 to 10 ring atoms. Illustrative examples of such a fused bicyclic heterocycloalkyl group include:

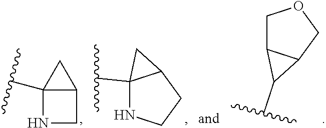

In another embodiment, a heterocycloalkyl group can be a bridged heterocycloalkyl group having from 6 to 10 ring atoms. Illustrative examples of such a bridged bicyclic heterocycloalkyl group include:

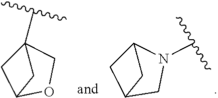 [SMILES: *C12COC(C1)C2 ; *N1CC2CC1C2]

The term “heterocycloalkenyl,” as used herein, refers to a heterocycloalkyl group, as defined above, wherein the heterocycloalkyl group contains from 4 to 10 ring atoms, and at least one endocyclic carbon-carbon or carbon-nitrogen double bond. A heterocycloalkenyl group can be joined via a ring carbon or ring nitrogen atom. In one embodiment, a heterocycloalkenyl group has from 4 to 6 ring atoms. In another embodiment, a heterocycloalkenyl group is monocyclic and has 5 or 6 ring atoms. In one embodiment, a heterocycloalkenyl group is monocyclic. In another embodiment, a heterocycloalkenyl group is bicyclic. A heterocycloalkenyl group can optionally substituted by one or more ring system substituents, wherein “ring system substituent” is as defined above. The nitrogen or sulfur atom of the heterocycloalkenyl can be optionally oxidized to the corresponding N-oxide, S-oxide or S,S-dioxide. A ring carbon atom of a heterocycloalkenyl group may be functionalized as a carbonyl group (i.e., an oxo substituent). Non-limiting examples of heterocycloalkenyl groups include 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrothiazolyl, 3,4-dihydro-2H-pyranyl, dihydrofuranyl, fluoro-substituted dihydrofuranyl, 7-oxabicyclo[2.2.1]heptenyl, dihydrothiophenyl, dihydrothiopyranyl, and the like and the like. In one embodiment, a heterocycloalkenyl group is a 5-membered heterocycloalkenyl. In another embodiment, a heterocycloalkenyl group is a 6-membered heterocycloalkenyl. The term “4 to 6-membered heterocycloalkenyl” refers to a heterocycloalkenyl group having from 4 to 6 ring atoms. An illustrative example of a monocyclic heterocycloalkenyl group is:

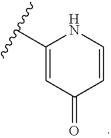 [SMILES: *c1cc(=O)cc[nH]1]

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound’ or “stable structure” is meant a compound that is sufficiently robust to survive isolation to a useful degree of purity from a reaction mixture, and formulation into an efficacious therapeutic agent.

The term “in substantially purified form,” as used herein, refers to the physical state of a compound after the compound is isolated from a synthetic process (e.g., from a reaction mixture), a natural source, or a combination thereof. The term “in substantially purified form,” also refers to the physical state of a compound after the compound is obtained from a purification process or processes described herein or well-known to the skilled artisan (e.g., chromatography, recrystallization and the like), in sufficient purity to be characterizable by standard analytical techniques described herein or well-known to the skilled artisan.

It should also be noted that any carbon as well as heteroatom with unsatisfied valences in the text, schemes, examples and tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, Greene et al., Protective Groups in Organic Synthesis, Wiley-Interscience, New York, (1999).

Examples of “ring system substituents” include, but are not limited to, alkyl, alkenyl, alkynyl, aryl, heteroaryl, -alkylene-aryl, -arylene-alkyl, -alkylene-heteroaryl, -alkenylene-heteroaryl, -alkynylene-heteroaryl, —OH, hydroxyalkyl, haloalkyl, —O-alkyl, —O-haloalkyl, -alkylene-O-alkyl, —O-aryl, —O-alkylene-aryl, acyl, —C(O)-aryl, halo, —NO₂, —CN, —SF₅, —C(O)OH, —C(O)O-alkyl, —C(O)O-aryl, —C(O)O-alkylene-aryl, —S(O)-alkyl, —S(O)₂-alkyl, —S(O)-aryl, —S(O)₂-aryl, —S(O)-heteroaryl, —S(O)z-heteroaryl, —S-alkyl, —S-aryl, —S-heteroaryl, —S-alkylene-aryl, —S-alkyleneheteroaryl, —S(O)₂-alkylene-aryl, —S(O)₂-alkylene-heteroaryl, —Si(alkyl)₂, —Si(aryl)₂, Si(heteroaryl)₂-Si(alkyl)(aryl), —Si(alkyl)(cycloalkyl), —Si(alkyl)(heteroaryl), cycloalkyl, heterocycloalkyl, —O—C(O)-alkyl, —O—C(O)-aryl, —O—C(O)-cycloalkyl, —C(═N—CN)—NH₂, —C(═NH)—NH₂, —C(═NH)—NH(alkyl), —N(Y¹)(Y²), -alkylene-N(Y¹)(Y²), —C(O)N(Y¹)(Y²), and —S(O)₂N(Y¹)(Y²), wherein Y¹ and Y² can be the same or different and are independently selected from the group consisting of hydrogen, alkyl, aryl, cycloalkyl, and -alkylene-aryl. “Ring system substituent” may also mean a single moiety which simultaneously replaces two available hydrogens on two adjacent carbon atoms (one H on each carbon) on a ring system. Examples of such moiety are methylenedioxy, ethylenedioxy, —C(CH₃)₂— and the like which form moieties such as, for example:

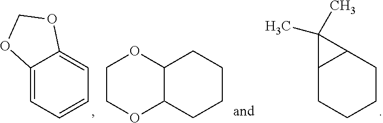

When any substituent or variable (e.g., R⁵, n, etc.) occurs more than one time in any constituent or in Formula (I), its definition on each occurrence is independent of its definition at every other occurrence, unless otherwise indicated.

As used herein, the term “composition” is intended to encompass a product comprising the specified ingredients in the specified amounts, as well as any product which results from combination of the specified ingredients in the specified amounts.

Prodrugs and solvates of the compounds of the present disclosure are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to provide a Substituted Benzothiophene Derivative or a pharmaceutically acceptable salt or solvate of the compound. The transformation may occur by various mechanisms (e.g., by metabolic or chemical processes), such as, for example, through hydrolysis in blood.

For example, if a Substituted Benzothiophene Derivative or a pharmaceutically acceptable salt, hydrate or solvate thereof contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 6 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di (C₁-C₂)alkylcarbamoyl-(C₁-C₂)alkyl and piperidino-, pyrrolidino- or morpholino(C₂-C₃)alkyl, and the like.

Similarly, if a Substituted Benzothiophene Derivative contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkyl, α-amino(C₁-C₄)alkylene-aryl, arylacyl and α-aminoacyl, or α-aminoacyl-α-aminoacyl, where each α-aminoacyl group is independently selected from the naturally occurring L-amino acids, —P(O)(OH)₂, —P(O)(O(C₁-C₆)alkyl)₂ or glycosyl (the radical resulting from the removal of a hydroxyl group of the hemiacetal form of a carbohydrate), and the like.

If a Substituted Benzothiophene Derivative incorporates an amine functional group, a prodrug can be formed by the replacement of a hydrogen atom in the amine group with a group such as, for example, R-carbonyl-, RO-carbonyl-, NRR′-carbonyl- wherein R and R′ are each independently (C₁-C₁₀)alkyl, (C₃-C₇) cycloalkyl, benzyl, a natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl; carboxy (C₁-C₆)alkyl; amino(C₁-C₄)alkyl or mono-N— or di-N,N—(C₁-C₆)alkylaminoalkyl; —C(Y⁴)Y⁵ wherein Y⁴ is H or methyl and Y⁵ is mono-N— or di-N,N—(C₁-C₆)alkylamino morpholino; piperidin-1-yl or pyrrolidin-1-yl, and the like.

Pharmaceutically acceptable esters of the present compounds include the following groups: (1) carboxylic acid esters obtained by esterification of the hydroxy group of a hydroxyl compound, in which the non-carbonyl moiety of the carboxylic acid portion of the ester grouping is selected from straight or branched chain alkyl (e.g., methyl, ethyl, n-propyl, isopropyl, t-butyl, sec-butyl or n-butyl), alkoxyalkyl (e.g., methoxymethyl), aralkyl (e.g., benzyl), aryloxyalkyl (for example, phenoxymethyl), aryl (e.g., phenyl optionally substituted with, for example, halogen, C₁₋₄alkyl, —O—(C₁₋₄alkyl) or amino); (2) sulfonate esters, such as alkyl- or aralkylsulfonyl (for example, methanesulfonyl); (3) amino acid esters (e.g., L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

One or more compounds of the present disclosure may exist in unsolvated as well as solvated forms with pharmaceutically acceptable solvents such as water, ethanol, and the like, and it is intended that the present disclosure embrace both solvated and unsolvated forms. “Solvate” means a physical association of a compound of this disclosure with one or more solvent molecules. This physical association involves varying degrees of ionic and covalent bonding, including hydrogen bonding. In certain instances, the solvate will be capable of isolation, for example when one or more solvent molecules are incorporated in the crystal lattice of the crystalline solid. “Solvate” encompasses both solution-phase and isolatable solvates. Non-limiting examples of solvates include ethanolates, methanolates, and the like. A “hydrate” is a solvate wherein the solvent molecule is water.

One or more compounds of the present disclosure may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTechours., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than room temperature, and cooling the solution at a rate sufficient to form crystals which are then isolated by standard methods. Analytical techniques such as, for example IR spectroscopy, show the presence of the solvent (or water) in the crystals as a solvate (or hydrate).

The Substituted Benzothiophene Derivatives can form salts which are also within the scope of this disclosure. The term “salt(s)”, as employed herein, denotes acidic salts formed with inorganic and/or organic acids, as well as basic salts formed with inorganic and/or organic bases. In addition, when a Substituted Benzothiophene Derivative contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. In one embodiment, the salt is a pharmaceutically acceptable (i.e., non-toxic, physiologically acceptable) salt. In another embodiment, the salt is other than a pharmaceutically acceptable salt. Salts of the Compounds of Formula (I) may be formed, for example, by reacting a Substituted Benzothiophene Derivative with an amount of acid or base, such as an equivalent amount, in a medium such as one in which the salt precipitates or in an aqueous medium followed by lyophilization.

Exemplary acid addition salts include acetates, ammonium, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates (also known as mesylates), naphthalenesulfonates, nitrates, oxalates, phosphates, propionates, salicylates, succinates, sulfates, tartarates, thiocyanates, toluenesulfonates (also known as tosylates), and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto. In one embodiment, an acid salt is an ammonium salt or a di-ammonium salt.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamine, t-butyl amine, choline, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

All such acid salts and base salts are intended to be pharmaceutically acceptable salts within the scope of the present disclosure and all acid and base salts are considered equivalent to the free forms of the corresponding compounds for purposes of the present disclosure.

Diastereomeric mixtures can be separated into their individual diastereomers on the basis of their physical chemical differences by methods well-known to those skilled in the art, such as, for example, by chromatography and/or fractional crystallization. Enantiomers can be separated using converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Stereochemically pure compounds may also be prepared by using chiral starting materials or by employing salt resolution techniques. Also, some of the Substituted Benzothiophene Derivatives may be atropisomers (e.g., substituted biaryls), and are considered as part of this disclosure. Enantiomers can also be directly separated using chiral chromatographic techniques.

It is also possible that the Substituted Benzothiophene Derivatives may exist in different tautomeric forms, and all such forms are embraced within the scope of the present disclosure. For example, all keto-enol and imine-enamine forms of the compounds are included in the present disclosure.

All stereoisomers (for example, geometric isomers, optical isomers and the like) of the present compounds (including those of the salts, solvates, hydrates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this disclosure. If a Substituted Benzothiophene Derivative incorporates a double bond or a fused ring, both the cis- and trans-forms, as well as mixtures, are embraced within the scope of the present disclosure.

Individual stereoisomers of the compounds of the present disclosure may, for example, be substantially free of other isomers, or may be admixed, for example, as racemates or with all other, or other selected, stereoisomers. The chiral centers of the present disclosure can have the S or R configuration as defined by the IUPAC 1974 Recommendations. The use of the terms “salt”, “solvate”, “ester”, “prodrug” and the like, is intended to apply equally to the salt, solvate, ester and prodrug of enantiomers, stereoisomers, rotamers, tautomers, positional isomers, racemates or prodrugs of the inventive compounds.

In the Compounds of Formula (I), the atoms may exhibit their natural isotopic abundances, or one or more of the atoms may be artificially enriched in a particular isotope having the same atomic number, but an atomic mass or mass number different from the atomic mass or mass number predominantly found in nature. The present disclosure is meant to include all suitable isotopic variations of the compounds of generic Formula I. For example, different isotopic forms of hydrogen (H) include protium (H), and deuterium (H). Protium is the predominant hydrogen isotope found in nature. Enriching for deuterium may provide certain therapeutic advantages, such as increasing in vivo half-life or reducing dosage requirements, or may provide a compound useful as a standard for characterization of biological samples. Isotopically-enriched Compounds of Formula (I) can be prepared without undue experimentation by conventional techniques well known to those skilled in the art or by processes analogous to those described in the Schemes and Examples herein using appropriate isotopically-enriched reagents and/or intermediates. In one embodiment, a Compound of Formula (I) has one or more of its hydrogen atoms replaced with deuterium.

Polymorphic forms of the Substituted Benzothiophene Derivatives, and of the salts, solvates, hydrates, esters and prodrugs of the Substituted Benzothiophene Derivatives, are intended to be included in the present disclosure.

The following abbreviations are used below and have the following meanings: Ac is acetyl; ACN or MeCN is acetonitrile; AcOH is acetic acid; AIBN is azobisisobutyronitrile; APhos-Pd-G3 is [4-(Di-tert-butylphosphino)-N,N-dimethylaniline-2-(2′-aminobiphenyl)]palladium(II) methanesulfonate; APhos-Pd-G4 is 4-ditert-butylphosphanyl-N,N-dimethylaniline; methanesulfonic acid; N-methyl-2-phenylaniline; palladium; Boc is tert-butyloxycarbonyl; Boc-Ser(Bzl)-OH is N-(tert-Butoxycarbonyl)-O-benzyl-L-serine; BPO is benzoyl peroxide; cataCXium A® is Di(1-adamantyl)-n-butylphosphine; CDI is 1,1′-carbonyldiimidazole; Celite is diatomaceous earth; ChiralArt Cellulose-SB is cellulose tris(3,5-dimethylphenylcarbamate) coated on silica gel; ChiralPak AD-H is amylose tris-(3,5-dimethylphenylcarbamate) coated on 5 μm silica-gel; ChiralPak AS is amylose-tris(S)-α-methylphenylcarbamate coated on silica-gel; ChiralPak IE is amylose tris-(3,5-dichlorophenylcarbamate) immobilised on 5 μm silica-gel; CyJohnPhos is 2-(Dicyclohexylphosphino)biphenyl; Cphos Pd G4 is 2-(2-dicyclohexylphosphanylphenyl)-1-N,1-N,3-N,3-N-tetramethylbenzene-1,3-diamine methanesulfonic acid N-methyl-2-phenylaniline palladium (II); DAST is Diethylaminosulfur trifluoride; DCE is dichloroethane; DCM is dichloromethane; Deoxofluor is Bis(2-methoxyethyl)aminosulfur trifluoride; Dess-Martin periodinane is 1,1,1-Tris(acetyloxy)-1,1-dihydro-1,2-benziodoxol-3-(1H)-one; DIAD is Diisopropyl azodicarboxylate; DIEA or DIPEA is diisopropylethylamine; DMA is Dimethylacetamide; DMAP is 4-Dimethylaminopyridine DME is dimethoxyethane; DMF is N,N-dimethylformamide; DMSO is dimethylsulfoxide; DPPE is 1,2-bis(diphenylphosphino)ethane; EDCI is 1-ethyl-3-(3-dimethylaminopropyl) carbodiimide; ESI is electrospray ionization; Et is ethyl, EtOH is ethanol; Et₃N or TEA is triethylamine; EtOAc is ethyl acetate; HATU is (1-[bis(dimethylamino)methylene]-1H-1,2,3-triazolo[4,5-b]pyridinium 3-oxide hexafluorophosphate; HFBA is heptafluorobutyric acid; HOBT is hydroxybenzotriazole; HPLC is high performance liquid chromatography; LAH or LiAlH₄ is lithium aluminum hydride; LCMS is liquid chromatography/mass spectrometry; LDA is lithium diisopropylamide; LG is leaving group; LHMDS is lithium hexamethyldisilazane; Lux Cellulose is cellulose tris-(3,5-dimethylphenylcarbamate) coated on silica-gel; mCPBA is meta-chloroperoxybenzoic acid; Me is methyl; MeOH is methanol; Ms is mesyl; MS is mass spectrometry; MTBE is methyl tert-butyl ether; NBS is N-bromosuccinimide; NCS is N-chlorosuccinimide; NMP is N-methylpyrrolidinone; OMs is mesylate; OTs is tosylate; OTf is triflate; OXONE is Potassium peroxymonosulfate; PMB is para-methoxy benzyl; Pd/C is palladium on carbon; Pd(OH)₂/C (Pearlman's catalyst) is palladium hydroxide on carbon; Pd₂(dba)₃ is Tris(dibenzylideneacetone) dipalladium(0); Pd(dppf)Cl₂ is [1,1′-Bis(diphenylphosphino)ferrocene]palladium(II) dichloride; Pd(dtbpf) Cl₂ is [1,1′-Bis(di-tert-butylphosphino)ferrocene]dichloropalladium(II); Pd(Ph₃P)₄ is Tetrakis(triphenylphosphine)palladium(0); Prep TLC is preparative thin layer chromatography; (R,R)-WHELK-01-Kromasil is 1-(3,5-dinitrobenzamido)-1,2,3,4,-tetrahydrophenanthrene coated on silica gel; RuPhos Pd G3 or RuPhos-G3-Palladacycle is (2-Dicyclohexylphosphino-2′,6′-diisopropoxy-1,1′-biphenyl)[2-(2′-amino-1,1′-biphenyl)]palladium(II) methanesulfonate; SEM is 2-(trimethylsilyl)ethoxymethyl; SFC is supercritical fluid chromatography; SPhos Pd G2 is chloro(sodium-2-dicyclohexylphosphino-2′,6′-dimethoxy-1,1′-biphenyl-3′-sulfonate)[2-(2′-amino-1,1′-biphenyl)]palladium(II); tBu is tert-butyl; tert-butyl Xphos Pd G3 is [(2-Di-tert-butylphosphino-2′,4′,6′-triisopropyl-1,1′-biphenyl)-2-(2′-amino-1,1′-biphenyl)]palladium(II) methanesulfonate; TBAF is Tetrabutylammonium fluoride; TBS is tert-butyl(chloro)dimethylsilane; TEA is triethylamine; Tf is triflyl; TFA is trifluoroacetic acid; THF is tetrahydrofuran; TLC is thin-layer chromatography; TMS-Br is trimethylsilyl bromide; Ts is tosyl; Xantphos is 4,5-bis(diphenylphosphino)-9,9-dimethylxanthene; and Xphos-Pd G2 or X-Phos aminobiphenyl palladium chloride precatalyst is Chloro(2-dicyclohexylphosphino-2′,4′,6′-triisopropyl-1,1′-biphenyl)[2-(2′-amino-1,1′-biphenyl)]palladium(II).

### The Compounds of Formula (I)

Provided herein are Substituted Benzothiophene Derivatives of Formula (I):

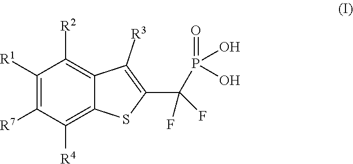

and pharmaceutically acceptable salts thereof, wherein R¹, R², R³, R⁴, and R⁷ are defined above for the Compounds of Formula (I).

In one embodiment, for the compounds of formula (I), R¹ is selected from H, —C(O)N(R⁵)(R⁶), 5 or 6-membered monocyclic heteroaryl, —CN, —NH₂, NHCOCF₃, —CH═NHNH₂, 9 or 10-membered bicyclic heterocycloalkyl, —CHNH₂CF₃, —CH(NH)phenyl, —NHC(O)—(C₁-C₁₀ alkenyl)ₙ-(5 or 6-membered monocyclic heteroaryl), and —O—(C₁-C₁₀ alkylene)-(C₆-C₁₀ aryl), In another embodiment, for the compounds of formula (I), R¹ is —C(O)N(R⁵)(R⁶) or 5 or 6-membered heteroaryl.

In another embodiment, for the compounds of formula (I), R¹ is —C(O)N(R⁵)(R⁶), wherein R⁵ is H or methyl, and R⁶ is selected from H, C₁-C₁₀ alkyl, and —(C₁-C₁₀ alkylene)ₙ-5 or 6-membered monocyclic heteroaryl.

In still another embodiment, for the compounds of formula (I), R¹ is —C(O)NH(R⁶), wherein R⁶ is selected from H, C₁-C₁₀ alkyl, and —(C₁-C₁₀ alkylene)ₙ-5 or 6-membered monocyclic heteroaryl.

In one embodiment, for the compounds of formula (I), R² is H.

In one embodiment, for the compounds of formula (I), R³ is selected from Br, Cl, —CN—CH₃, —CHF₂, and CF₃.

In another embodiment, for the compounds of formula (I), R³ is Br.

In one embodiment, for the compounds of formula (I), R⁴ is selected from H, halo, —O—(C₁-C₁₀ haloalkyl), —O—(C₁-C₁₀ hydroxyalkyl), —O—(C₁-C₁₀ alkylene)-CN, —O—(C₁-C₁₀ alkylene)-S(O)₂—(C₁-C₁₀ alkyl), —O—(C₁-C₁₀ alkylene)-S(O)₂NH—(C₁-C₁₀ alkyl), —O—(C₁-C₁₀ alkylene)-(C₆-C₁₀ aryl), —O—(C₁-C₁₀ alkylene)-(C₃-C₇ monocyclic cycloalkyl), —O—(C₁-C₁₀ alkylene)-(3 to 7-membered monocyclic heterocycloalkyl), —O—(C₁-C₁₀ alkylene)-(5 or 6-membered monocyclic heteroaryl), —O—(C₁-C₁₀ alkyl), —NH—(C₁-C₁₀ alkyl), —NH—(C₁-C₁₀ haloalkyl), —NH—(C₁-C₁₀ alkylene)-(C₃-C₇ monocyclic cycloalkyl), —O—(C₁-C₁₀ alkylene)-C(O)NH₂, —O—(C₁-C₁₀ alkylene)-C(O)NH—(5 or 6-membered monocyclic heteroaryl), —O—(C₁-C₁₀ alkylene)ₙ-(5 to 10 membered bridged cycloalkyl), —O—(C₁-C₁₀ haloalkyl), (9 or 10-membered bicyclic heteroaryl), and —O—(C₁-C₁₀ alkylene)ₙ-(3 to 7-membered monocyclic heterocycloalkyl), wherein said C₃-C₇ monocyclic cycloalkyl can be optionally substituted with 1-3 groups, each independently selected from halo and —CN.

In one embodiment, for the compounds of formula (I), R⁷ is selected from H, Br, F, CN and CON(R⁸)₂, wherein R⁸ is H, C₁-C₆ alkyl, or C₆-C₁₀ aryl.

In one embodiment, for the compounds of formula (I), R¹ is selected from: I, —OH, —C(O)NH₂, —C(O)NHCH₃, —C—CH, —C(O)NHCH₂CH₃, —C(O)NHCD₃-C(O)OH, —CH₂OH, —CH₂CN, pyrazolyl, —CH₂S(O)(NH)—CH₃, —S(O)(NH)—CH₃, imidazolyl, —C(O)NHCH₂CN, —C(O)NHCH₂CH₂CN, —S(O)₂NHCH₃, —CH₂NHC(O)CH₃,

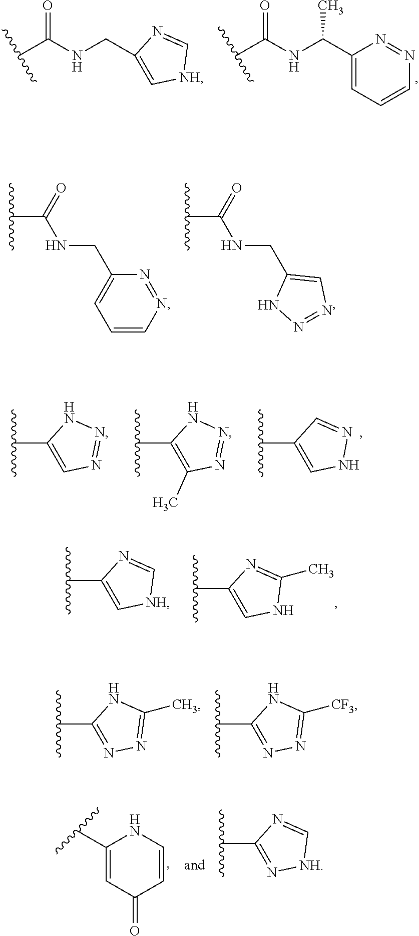

In one embodiment, for the compounds of formula (I), R⁴ is selected from: H, —O(CH₂)₃CF₃, —O(CH₂)₂C(CH₃)₂OH, —O(CH₂)₃SO₂CH₃, —O(CH₂)₃SO₂NH₂, —O(CH₂)₄C(O)NH₂, —OCH(CH₃)CH₂CH₂CH₃, —O(CH₂)₃SO₂N(CH₃)₂, —O(CH₂)₃SO₂NHCH₃, —O(CH₂)₄SO₂CH₃, —(CH₂)₄SO₂NH₂, —NH(CH₂)SO₂NH₂, —O(CH₂)₄C(O)NH₂, —O(CH₂)₂NHSO₂CH₃, —O(CH₂)₂CF₂CH₃, —O—CH(CH₃)CH₂CH₂CH₃, pyrazolyl, —S(CH₂)₃CH₃, —CH₂CH₂-phenyl, —C—C(CH₂)₂SO₂NH₂, —CH₂CH₂—Si(CH₃)₃, —O(CH₂)₃SO₂CF₃, —OCH(CH₂OH)CH₂CH₂CH₃, —OCH₂CH(F)CH₂CF₃,

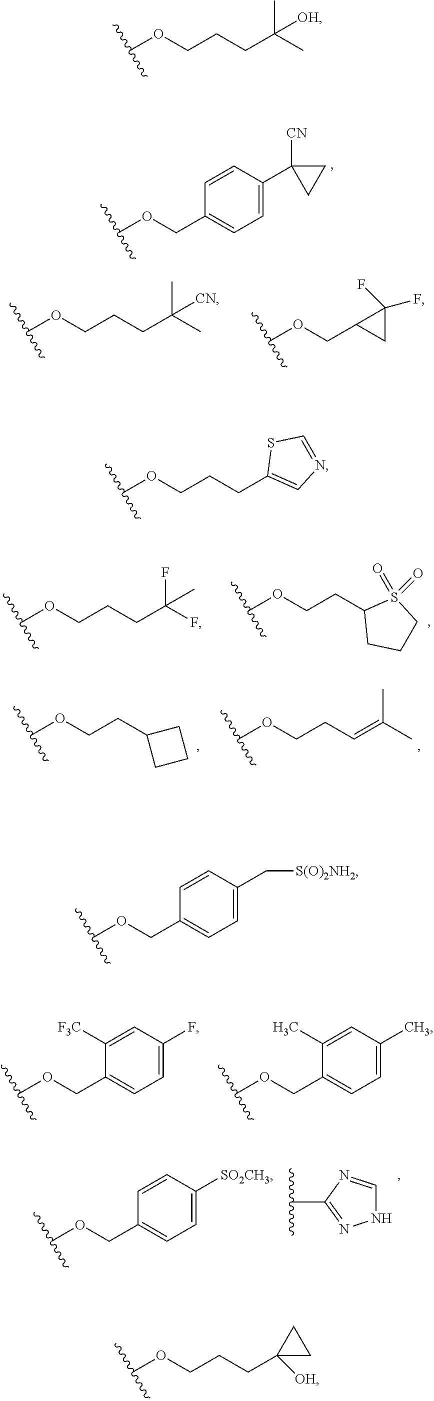

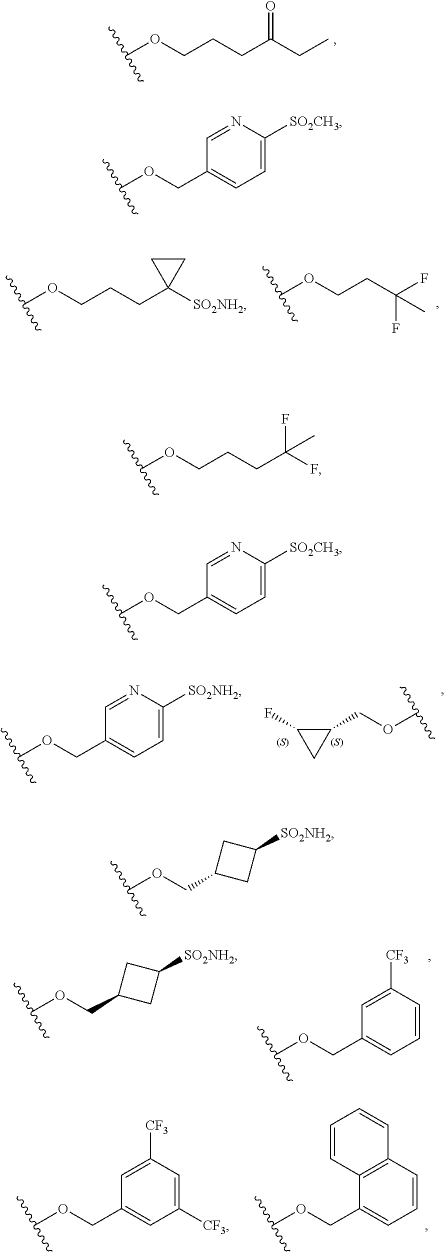

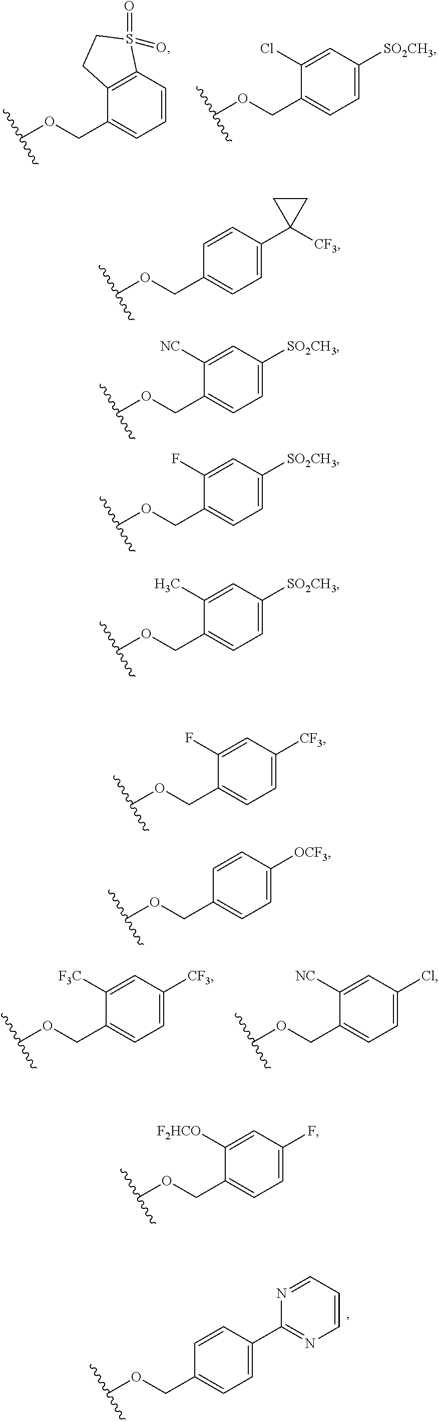

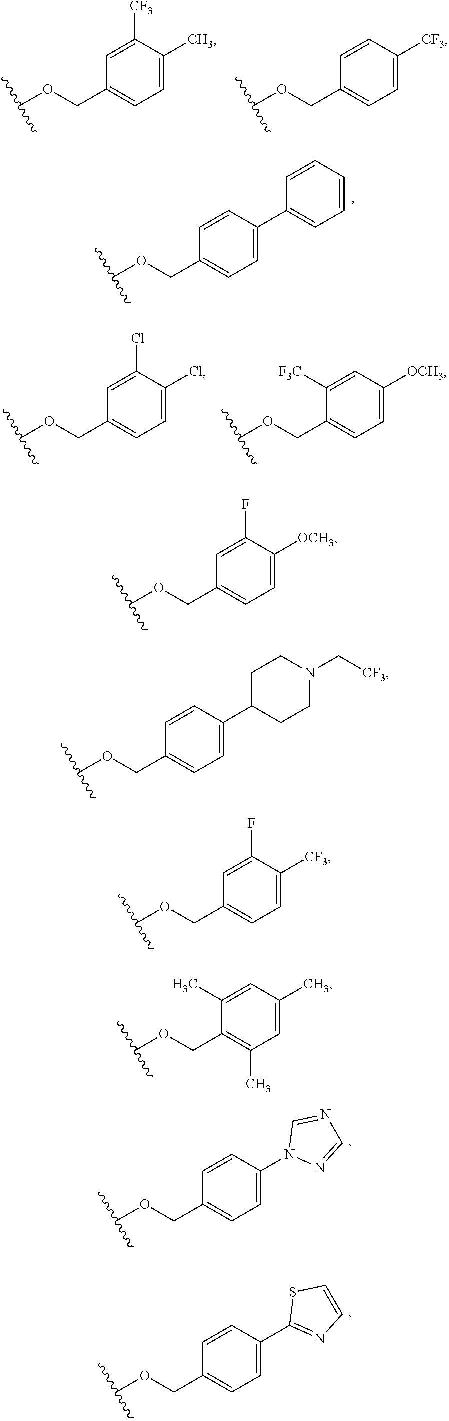

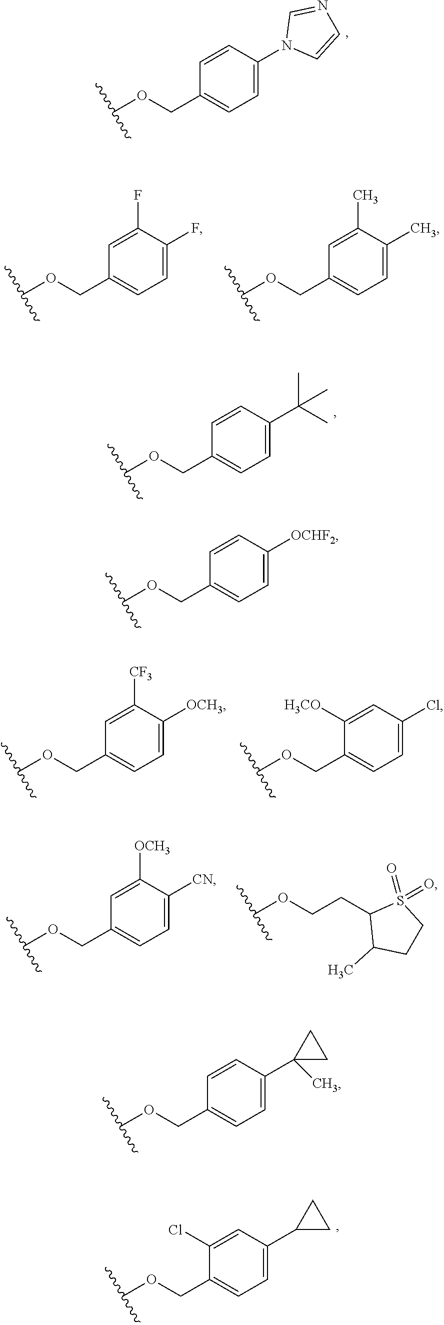

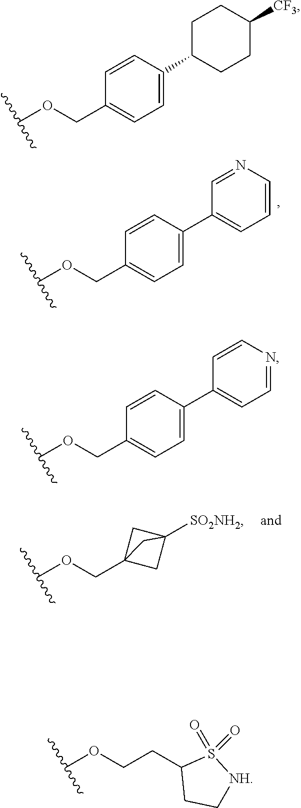 [SMILES: *OCc1ccc([C@H]2CC[C@H](C(F)(F)F)CC2)cc1 ; *OCc1ccc(-c2cccnc2)cc1 ; *OCc1ccc(-c2ccncc2)cc1 ; *OCC12CC(S(N)(=O)=O)(C1)C2 ; *OCCC1CCNS1(=O)=O]

In one embodiment, the compounds of formula (I) have the formula (Ia):

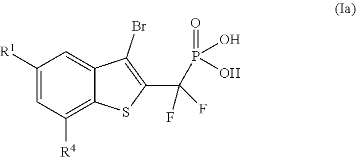

or a pharmaceutically acceptable salt thereof,
wherein:
R¹ is —C(O)NH(R⁶) or 5-membered monocyclic heteroaryl,
R⁴ is H, halo, —O—(C₁-C₁₀ haloalkyl), —O—(C₁-C₁₀ hydroxyalkyl), —O—(C₁-C₁₀ alkylene)-S(O)₂—(C₁-C₁₀ alkyl), —O—(C₁-C₁₀ alkylene)-S(O)₂NH—(C₁-C₁₀ alkyl), —O—(C₁-C₁₀ alkylene)ₙ-(3 to 7-membered monocyclic heterocycloalkyl); and
R⁶ is selected from H, C₁-C₁₀ alkyl, and —(C₁-C₁₀ alkylene)ₙ-5 or 6-membered monocyclic heteroaryl; and each occurrence of n is independently selected from 0 or 1.

In one embodiment, for the compounds of formula (I) or (Ia), R¹ is —C(O)NH(R⁶).

In another embodiment, for the compounds of formula (I) or (Ia), R¹ is —C(O)NH(R⁶), and R⁶ is —(C₁-C₁₀ alkylene)ₙ-(5 or 6-membered monocyclic heteroaryl).

In another embodiment, for the compounds of formula (I) or (Ia), R¹ is 5-membered heteroaryl.

In one embodiment, for the compounds of formula (I) or (Ia), R¹ is selected from: —C(O)NH₂, —C(O)NHCH₃,

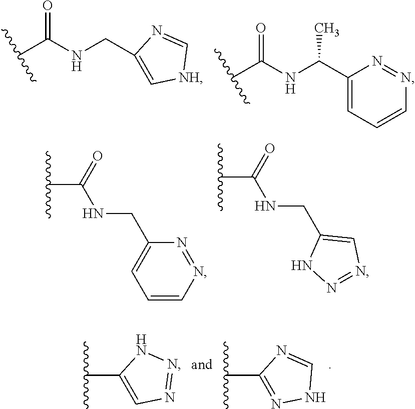

In one embodiment, for the compounds of formula (I) or (Ia), R⁴ is selected from: H, —O(CH₂)₃CF₃, —O(CH₂)₂C(CH₃)₂OH, —O(CH₂)₃SO₂CH₃, —O(CH₂)₃SO₂NH₂,

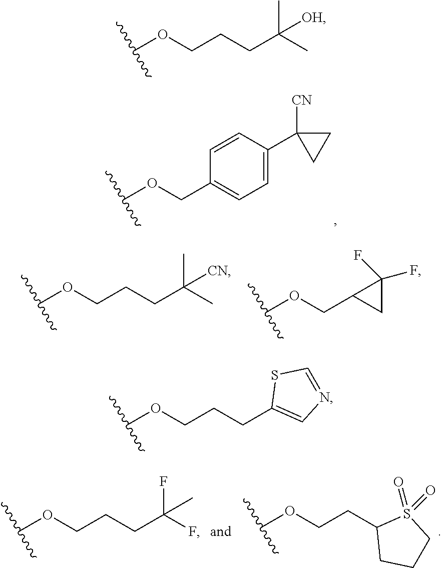

In one embodiment, the compound of formula (I) is any of the compounds numbered 1-464 in the instant specification, or a pharmaceutically acceptable salt thereof.

In one embodiment, the compound of formula (I) is in substantially purified form.

Other embodiments include the following:
(a) A pharmaceutical composition comprising an effective amount of a Substituted Benzothiophene Derivative, and a pharmaceutically acceptable carrier.
(b) The pharmaceutical composition of (a), further comprising a second therapeutic agent selected from the group consisting of anticancer agents.
(c) The pharmaceutical composition of (b), wherein the anticancer agent is an anti-human PD-1 antibody (or antigen-binding fragment thereof).
(d) A pharmaceutical combination that comprises: (i) a Substituted Benzothiophene Derivative, and (ii) a second therapeutic agent selected from the group consisting of anticancer agents, wherein the Substituted Benzothiophene Derivative, and the second therapeutic agent are each employed in an amount that renders the combination effective for inhibiting replication of cancer cells, or for treating cancer and/or reducing the likelihood or severity of symptoms of cancer.
(e) The combination of (d), wherein the second therapeutic agent is an anti-human PD-1 antibody (or antigen-binding fragment thereof).
(f) A method of inhibiting cancer cell replication in a subject in need thereof which comprises administering to the subject an effective amount of a Substituted Benzothiophene Derivative.
(g) A method of treating cancer and/or reducing the likelihood or severity of symptoms of cancer in a subject in need thereof which comprises administering to the subject an effective amount of a Substituted Benzothiophene Derivative.
(h) The method of (g), wherein the Substituted Benzothiophene Derivative is administered in combination with an effective amount of at least one second therapeutic agent selected from the group consisting of anticancer agents.
(i) The method of (h), wherein the second therapeutic agent is an anti-human PD-1 antibody (or antigen-binding fragment thereof).
(j) A method of inhibiting cancer cell replication in a subject in need thereof which comprises administering to the subject the pharmaceutical composition of (a), (b) or (c) or the combination of (d) or (e).
(k) A method of treating cancer and/or reducing the likelihood or severity of symptoms of cancer in a subject in need thereof which comprises administering to the subject the pharmaceutical composition of (a), (b) or (c) or the combination of (d) or (e).

Also described herein are Substituted Benzothiophene Derivative for use (i) in, (ii) as a medicament for, or (iii) in the preparation of a medicament for: (a) medicine; (b) inhibiting cancer cell replication, or (c) treating cancer and/or reducing the likelihood or severity of symptoms of cancer. In these uses, the Substituted Benzothiophene Derivative can optionally be employed in combination with one or more additional therapeutic agents selected from anticancer agents.

It is further to be understood that the embodiments of compositions and methods provided as (a) through (k) above are understood to include all embodiments of the compounds, including such embodiments as result from combinations of embodiments.

Non-limiting examples of the Compounds of Formula (I) include compounds 1-464, as set forth in the Examples below, and pharmaceutically acceptable salts thereof.

### Methods for Making the Compounds of Formula (I)

The Compounds of Formula (I) may be prepared from known or readily prepared starting materials, following methods known to one skilled in the art of organic synthesis. Methods useful for making the Compounds of Formula (I) are set forth in the Examples below Alternative synthetic pathways and analogous structures will be apparent to those skilled in the art of organic synthesis.

One skilled in the art of organic synthesis will recognize that the synthesis of the bicyclic heterocycle cores contained in Compounds of Formula (I) may require protection of certain functional groups (i.e., derivatization for the purpose of chemical compatibility with a particular reaction condition). Suitable protecting groups for the various functional groups of these Compounds and methods for their installation and removal are well known in the art of organic chemistry. A summary of many of these methods can be found in Greene et al., Protective Groups in Organic Synthesis, Wiley-Interscience, New York, (1999).

One skilled in the art of organic synthesis will also recognize that one route for the synthesis of the bicyclic heterocycle cores of the Compounds of Formula (I) may be more desirable depending on the choice of appendage substituents.

Additionally, one skilled in the art will recognize that in some cases the order of reactions may differ from that presented herein to avoid functional group incompatibilities and thus adjust the synthetic route accordingly.

The preparation of multicyclic intermediates useful for making the bicyclic heterocycle cores of the Compounds of Formula (I) have been described in the literature and in compendia such as “Comprehensive Heterocyclic Chemistry” editions I, II and III, published by Elsevier and edited by A. R. Katritzky & R. J K Taylor. Manipulation of the required substitution patterns have also been described in the available chemical literature as summarized in compendia such as “Comprehensive Organic Chemistry” published by Elsevier and edited by DH R. Barton and W. D. Ollis; “Comprehensive Organic Functional Group Transformations” edited by edited by A. R. Katritzky & R. J K Taylor and “Comprehensive Organic Transformation” 3^{rd} Edition, published by Wiley-CVH and edited by R. C. Larock.

The starting materials used, and the intermediates prepared using the methods set forth in the Examples below may be isolated and purified if desired using conventional techniques, including but not limited to filtration, distillation, crystallization, chromatography and alike. Such materials can be characterized using conventional means, including physical constants and spectral data.

One skilled in the art will be aware of standard formulation techniques as set forth in the open literature as well as in textbooks such as Zheng, “Formulation and Analytical Development for Low-dose Oral Drug Products,” Wiley, 2009, ISBN.

### EXAMPLES

### General Methods

Solvents, reagents, and intermediates that are commercially available were used as received. Reagents and intermediates that are not commercially available were prepared in the manner as described below. ¹H NMR spectra are reported as ppm from residual solvent with number of protons, multiplicities, and coupling constants in Hertz indicated parenthetically. Where LC/MS data are presented, the observed parent ions are given. Unless otherwise noted, all reactions are magnetically stirred. Unless otherwise noted, flash chromatography is carried out on an Isco, Analogix, or Biotage automated chromatography system using a commercially available silica gel cartridge as the column. Reverse phase prep-HPLC conditions are described herein. When an aqueous solutions was concentrated, it was either concentrated on a Genevac evaporator or lyophilized.

### Synthesis of Intermediates

### Synthesis of Intermediate Compound Int-1

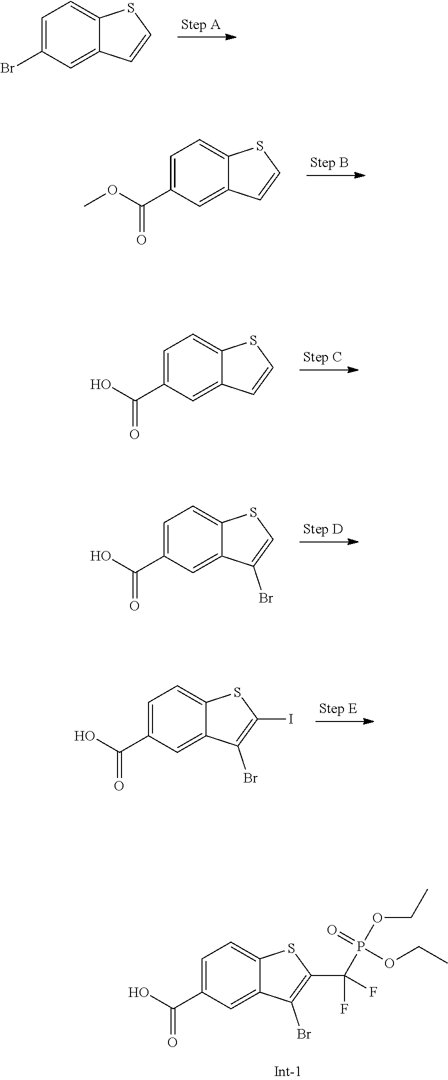 [SMILES: Brc1ccc2sccc2c1 ; COC(=O)c1ccc2sccc2c1 ; O=C(O)c1ccc2sccc2c1 ; O=C(O)c1ccc2scc(Br)c2c1 ; O=C(O)c1ccc2sc(I)c(Br)c2c1 ; CCOP(=O)(OCC)C(F)(F)c1sc2ccc(C(=O)O)cc2c1Br]

### Step A: Synthesis of methyl benzo[b]thiophene-5-carboxylate

A mixture of 5-bromobenzothiophene (400 g, 1.88 mol), Pd(dppf)Cl₂ (82.4 g, 112 mmol), and triethylamine (783 mL, 5.63 mol) in methanol (2.0 L) was degassed with nitrogen and then purged with CO. The mixture was stirred under a CO atmosphere for 12 hours at 100° C. The mixture was cooled to room temperature, filtered, then concentrated in vacuo, and the resulting residue was suspended in ethyl acetate (1.0 L). The mixture was filtered, and the filtrate was concentrated in vacuo to provide methyl benzo[b]thiophene-5-carboxylate, which was used without purification in the next step. ¹H NMR (400 MHz, DMSO-d₆) δ 8.54 (d, J=1.2 Hz, 1H), 8.15 (d, J=8.4 Hz, 1H), 7.86-7.96 (m, 2H), 7.63 (d, J=5.6 Hz, 1H), 3.90 (s, 3H).

### Step B: Synthesis of benzo[b]thiophene-5-carboxylic acid

A mixture of methyl benzo[b]thiophene-5-carboxylate (180 g, 936 mmol), and sodium hydroxide (74.9 g, 1.87 mol) in methanol (0.75 L), and water (0.15 L) was allowed to stir at room temperature for 12 hours. The mixture was partially concentrated in vacuo and then diluted with additional water (0.8 L). HCl (2.0 M) was added to the mixture until pH ˜4-5. The mixture was then filtered, and the collected solids were dried in vacuo to provide benzo[b]thiophene-5-carboxylic acid, which was used in the next step without purification. ¹H NMR (400 MHz, DMSO-d₆) δ 13.83-12.31 (br s, 1H), 8.52 (s, 1H), 8.12 (br d, J=8.0 Hz, 1H), 7.83-7.97 (m, 2H), 7.61 (br d, J=5.2 Hz, 1H).

### Step C: Synthesis of 3-bromobenzo[b]thiophene-5-carboxylic acid

A mixture of benzo[b]thiophene-5-carboxylic acid (160 g, 897 mmol), and bromine (186 g, 1.17 mol) in acetic acid (1.6 L) was allowed to stir at room temperature for 2 hours. The mixture was diluted with saturated aqueous ammonium chloride (1.0 L), and the mixture was filtered. The collected solids were washed with water (2×500 mL), and the collected solids were dried in vacuo to provide 3-bromobenzo[b]thiophene-5-carboxylic acid, which was used without purification in the next step. NMR (400 MHz, DMSO-d₆) δ 8.30 (d, J=1.2 Hz, 1H), 8.18 (d, J=8.4 Hz, 1H), 8.11 (s, 1H), 7.98 (dd, J=1.2, 8.4 Hz, 1H). MS (ESI, m/z): 255, 257 (M−H)⁻

### Step D: Synthesis of 3-bromo-2-iodobenzo[b]thiophene-5-carboxylic acid

LDA (2.0 M in THF, 382 mL, 0.76 mol) was added dropwise to a mixture of 3-bromobenzo[b]thiophene-5-carboxylic acid (82.0 g, 318 mmol) in THF (0.8 L) at −78° C. The mixture was allowed to stir at −78° C. for 3 hours, then a solution of iodine (121 g, 478 mmol) in THF (200 mL) was added at −78° C. The mixture was gradually warmed to room temperature, and stirred for 12 hours at room temperature. The reaction was quenched by the addition of water (1.0 L), then partially concentrated in vacuo. Citric acid was added to the mixture until pH 4-5. The mixture was filtered, and the collected solids were dried in vacuo to provide 3-bromo-2-iodobenzo[b]thiophene-5-carboxylic acid, which was used in the next step without purification. NMR (400 MHz, DMSO-d₆) δ 8.23 (s, 1H), 8.03 (d, J=8.4 Hz, 1H), 7.90-7.96 (m, 1H). MS (ESI, m/z): 381, 383 (M−H)⁻

### Step E: Synthesis of ((diethoxyphosphoryl)difluoromethyl)zinc(II) bromide

A mixture of zinc (27.7 g, 424 mmol), and iodine (5.99 g, 23.6 mmol) in DMA (200 mL) was allowed to stir for 12 minutes at room temperature. Diethyl (bromodifluoromethyl)phosphonate (63.0 g, 235 mmol) was added to the mixture. The mixture was allowed to stir at room temperature for an additional 1 hour. The mixture was used directly in the next step as a solution in DMA.

### Step F: Synthesis of 3-bromo-2-((diethoxyphosphoryl)difluoromethyl)benzo[b]thiophene-5-carboxylic acid

A mixture of ((diethoxyphosphoryl)difluoromethyl)zinc(II) bromide (1.18 M in DMA, 200 mL, 235 mmol) was added to a mixture of 3-bromo-2-iodobenzo[b]thiophene-5-carboxylic acid (30.0 g, 78.3 mmol), and CuBr (22.4 g, 156 mmol) in DMA (150 mL) at room temperature. The mixture was allowed to stir at room temperature for 12 hours. The reaction was quenched with 1.0 M HCl and then filtered through Celite. The filtrate was diluted with ethyl acetate (2 L), and washed with water (2×500 mL). The organic layer was separated, dried over anhydrous sodium sulfate, filtered, and concentrated in vacuo, and the resulting residue was purified using reverse phase HPLC (eluting acetonitrile in water, with HCl modifier) to provide 3-bromo-2-((diethoxyphosphoryl)difluoromethyl)benzo[b]thiophene-5-carboxylic acid (Int-1). NMR (400 MHz, DMSO-d₆) δ 13.39 (s, 1H), 8.46 (s, 1H), 8.32 (d, J=8.4 Hz, 1H), 8.12 (d, J=8.4 Hz, 1H), 4.18-4.32 (m, 4H), 1.29 (t, J=7.0 Hz, 6H). MS (ESI, m/z): 441, 443 (M−H)⁻

### Synthesis of Intermediate Compound Int-2

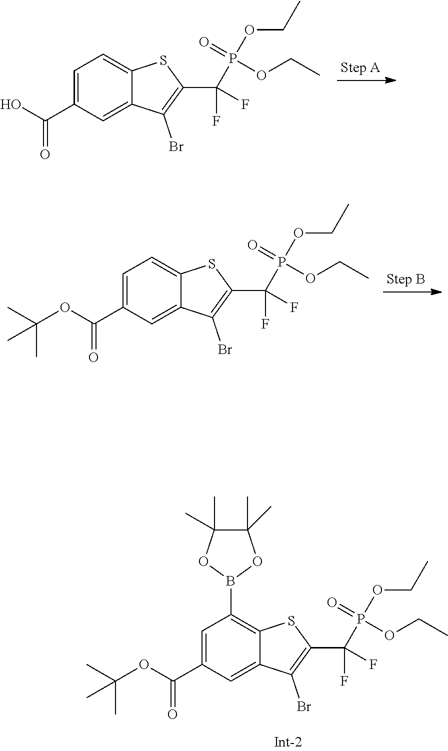

### Step A: Synthesis of tert-butyl 3-bromo-2-((diethoxyphosphoryl)difluoromethyl) benzo[b]thiophene-5-carboxylate

di-tert-Butyl dicarbonate (31.8 g, 146 mmol), and DMAP (2.14 g, 17.5 mmol) were added to a mixture of compound Int-1 (80.0 g, 58.3 mmol) in tert-butanol (480 mL) at room temperature. The mixture was heated to 50° C., and allowed to stir for 3 hours. The mixture was cooled to room temperature, diluted with ethyl acetate (2.0 L), and washed with saturated aqueous sodium bicarbonate (1.0 L). The organic layer was separated, dried over anhydrous sodium sulfate, filtered, and concentrated in vacuo, and the resulting residue was purified using silica gel chromatography (eluting ethyl acetate in petroleum ether) to provide tert-butyl 3-bromo-2-((diethoxyphosphoryl)difluoromethyl)benzo[b]thiophene-5-carboxylate. NMR (400 MHz, DMSO-d₆) δ 8.40 (s, 1H), 8.31 (d, J=8.4 Hz, 1H), 8.07 (d, J=8.4 Hz, 1H), 4.21-4.27 (m, 4H), 1.60 (s, 9H), 1.28 (t, J=7.2 Hz, 6H). MS (ESI, m/z): 499, 501 (M+H)⁺

### Step B: Synthesis of tert-butyl 3-bromo-2-((diethoxyphosphoryl)difluoromethyl)-7-(4,4,5,5-tetramethyl-1,3,2-dioxaborolan-2-yl)benzo[b]thiophene-5-carboxylate

A mixture of bis(1,5-cyclooctadiene)dimethoxydiiridium (66 mg, 0.10 mmol), and 4,4′-di-tert-butyl-2,2′-bipyridine (81 mg, 0.30 mmol) in cyclohexane (12 mL) was degassed with nitrogen (subsurface sparge) for 20 minutes. tert-butyl 3-bromo-2-((diethoxyphosphoryl)difluoromethyl)benzo[b]thiophene-5-carboxylate (2.00 g, 4.01 mmol), and bis(pinacolato)diboron (1.53 g, 6.01 mmol) were added to the reaction mixture. The mixture was degassed with nitrogen (subsurface sparge) for 20 minutes, then the reaction mixture was stirred and heated to 60° C. for 1 hour and then heated to 80° C. for an additional 1 hour. The mixture was concentrated in vacuo and The resulting residue was purified using silica gel chromatography (eluting ethyl acetate in petroleum ether). The isolated product was triturated with petroleum ether and filtered. The isolated solids were dried in vacuo to provide tert-butyl 3-bromo-2-((diethoxyphosphoryl)difluoromethyl)-7-(4,4,5,5-tetramethyl-1,3,2-dioxaborolan-2-yl)benzo[b]thiophene-5-carboxylate (Int-2). NMR (400 MHz, DMSO-d₆) δ 8.51 (d, J=1.2 Hz, 1H), 8.35 (d, J=1.2 Hz, 1H), 4.21-4.29 (m, 4H), 1.61 (s, 9H), 1.38 (s, 12H), 1.27-1.31 (m, 6H). MS (ESI, m/z): 625, 627 (M+H)⁺

### Synthesis of Intermediate Compound Int-3

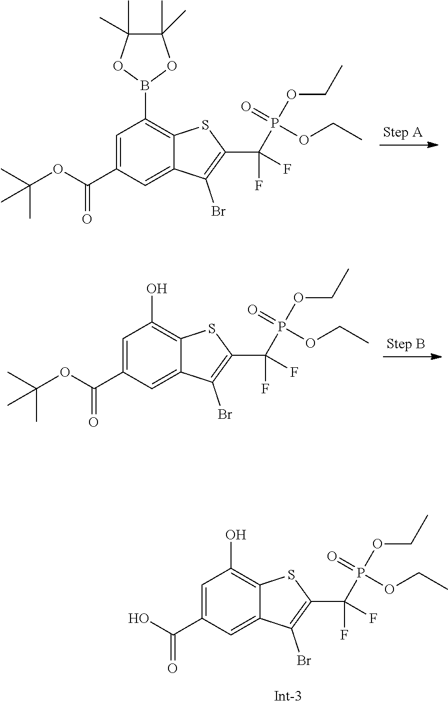

### Step A: Synthesis of tert-butyl 3-bromo-2-((diethoxyphosphoryl)difluoromethyl)-7-hydroxybenzo[b]thiophene-5-carboxylate

A mixture of OXONE (492 mg, 0.800 mmol) in water (10 mL) was added to a mixture of compound Int-2 (500 mg, 0.800 mmol) in acetone (10 mL) at room temperature. The mixture was allowed to stir at room temperature for 24 hours. The reaction mixture was diluted with ethyl acetate (250 mL), and washed with brine (50 mL). The organic layer was separated, dried over magnesium sulfate, filtered, and concentrated to provide tert-butyl 3-bromo-2-((diethoxyphosphoryl)difluoromethyl)-7-hydroxybenzo[b]thiophene-5-carboxylate, which was used without purification in the next step. MS (ESI, m/z): 515, 517 (M+H)⁺

### Step B: Synthesis of 3-bromo-2-((diethoxyphosphoryl)difluoromethyl)-7-hydroxybenzo[b]thiophene-5-carboxylic acid

TFA (1.8 mL, 23 mmol) was added to a mixture of tert-butyl 3-bromo-2-((diethoxyphosphoryl)difluoromethyl)-7-hydroxybenzo[b]thiophene-5-carboxylate (470 mg, 0.912 mmol) in dichloromethane (6 mL) at room temperature. The mixture was allowed to stir at room temperature for 2 hours. The reaction mixture was concentrated in vacuo to provide 3-bromo-2-((diethoxyphosphoryl)difluoromethyl)-7-hydroxybenzo[b]thiophene-5-carboxylic acid (Int-3), which was used without purification. MS (ESI, m/z): 459, 461 (M+H)⁺

### Synthesis of Intermediate Compound Int-4

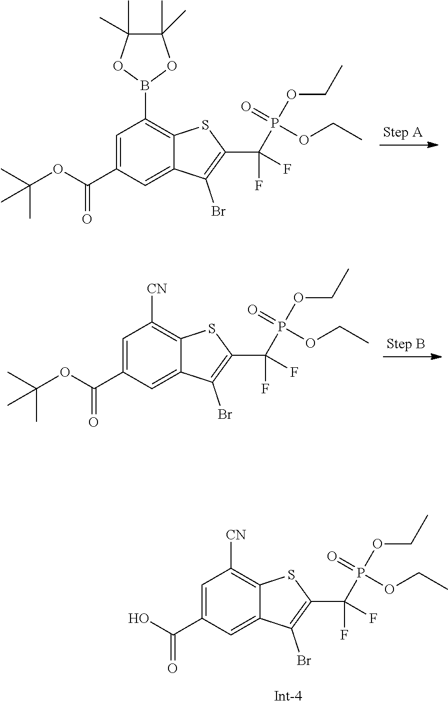

### Step A: Synthesis of tert-butyl 3-bromo-7-cyano-2-((diethoxyphosphoryl) difluoromethyl) benzo[b]thiophene-5-carboxylate

A mixture of tert-butyl 3-bromo-2-((diethoxyphosphoryl)difluoromethyl)-7-(4,4,5,5-tetramethyl-1,3,2-dioxaborolan-2-yl)benzo[b]thiophene-5-carboxylate (200 mg, 0.320 mmol), copper (II) nitrate trihydrate (155 mg, 0.640 mmol), zinc cyanide (113 mg, 0.960 mmol), and CsF (74 mg, 0.48 mmol) in methanol (2.3 mL), and water (0.9 mL) was stirred and heated to 80° C. for 3 hours. The reaction mixture was cooled to room temperature, and diluted with dichloromethane. The organic layer was separated, dried over magnesium sulfate, filtered, and concentrated in vacuo, and the resulting residue was purified using silica gel chromatography (eluting EtOAc/EtOH (3:1) in hexanes) to provide tert-butyl 3-bromo-7-cyano-2-((diethoxyphosphoryl)difluoromethyl)benzo[b]thiophene-5-carboxylate. MS (ESI, m/z): 524, 526 (M+H)⁺

### Step B: Synthesis of 3-bromo-7-cyano-2-((diethoxyphosphoryl)difluoromethyl)benzo[b]thiophene-5-carboxylic acid

TFA was added to a mixture of tert-butyl 3-bromo-7-cyano-2-((diethoxyphosphoryl)difluoromethyl)benzo[b]thiophene-5-carboxylate (100 mg, 0.191 mmol) in dichloromethane (1 mL) at room temperature. The mixture was allowed to stir at room temperature for 45 minutes. The mixture was concentrated in vacuo to provide 3-bromo-7-cyano-2-((diethoxyphosphoryl)difluoromethyl)benzo[b]thiophene-5-carboxylic acid (Int-4), which was used without purification in the next step. MS (ESI, m/z): 468, 470 (M+H)⁺.

### Synthesis of Intermediate Compound Int-5

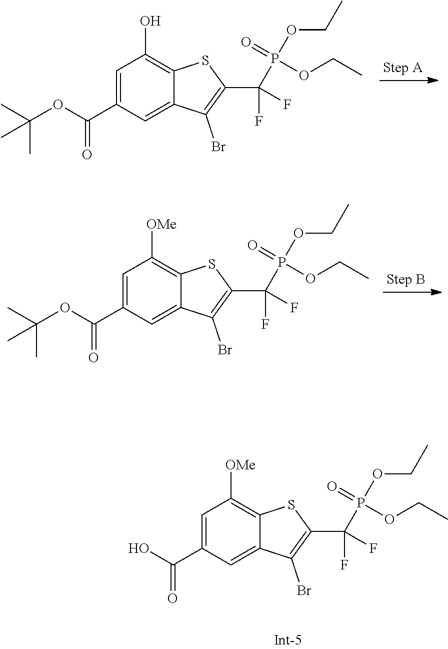

### Step A: Synthesis of tert-butyl 3-bromo-2-((diethoxyphosphoryl)difluoromethyl)-7-methoxybenzo[b]thiophene-5-carboxylate

Potassium carbonate (22 mg, 0.16 mmol), and iodomethane (25 μl, 0.40 mmol) were added to a mixture of tert-butyl 3-bromo-2-((diethoxyphosphoryl)difluoromethyl)-7-hydroxybenzo[b]thiophene-5-carboxylate (41 mg, 0.080 mmol) in DMF (0.8 mL). The mixture was allowed to stir at room temperature for 18 hours, then diluted with water and extracted with DCM. The organic layer was separated, dried over magnesium sulfate, filtered, and concentrated in vacuo to provide tert-butyl 3-bromo-2-((diethoxyphosphoryl)difluoromethyl)-7-methoxybenzo[b]thiophene-5-carboxylate, which was used without purification in the next step. MS (ESI, m/z): 529, 531 (M+H)⁺

### Step B: Synthesis of 3-bromo-2-((diethoxyphosphoryl)difluoromethyl)-7-methoxybenzo[b]thiophene-5-carboxylic acid

TFA (0.12 mL, 1.6 mmol) was added to a mixture of tert-butyl 3-bromo-2-((diethoxyphosphoryl)difluoromethyl)-7-methoxybenzo[b]thiophene-5-carboxylate (42 mg, 0.08 mmol) in DCM (0.5 mL) at room temperature. The mixture was allowed to stir at room temperature for 30 minutes. The mixture was concentrated in vacuo to provide 3-bromo-2-((diethoxyphosphoryl)difluoromethyl)-7-methoxybenzo[b]thiophene-5-carboxylic acid (Int-5), which was used without purification in the next step. MS (ESI, m/z): 473, 475 (M+H)⁺

### Synthesis of Intermediate Compound Int-6

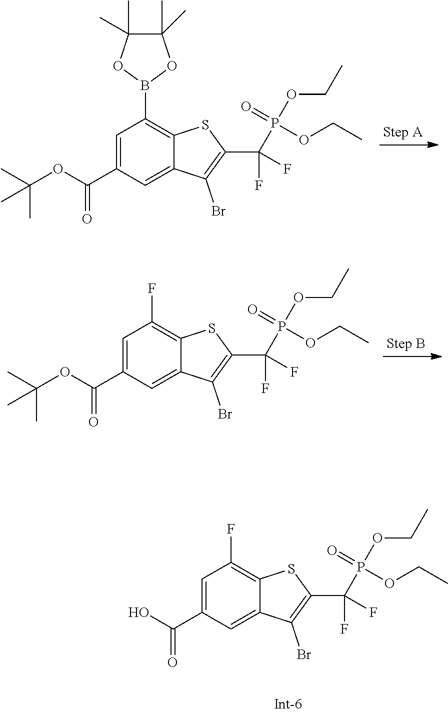

### Step A: Synthesis of tert-butyl 3-bromo-2-((diethoxyphosphoryl)difluoromethyl)-7-fluorobenzo[b]thiophene-5-carboxylate

A mixture of tert-butyl 3-bromo-2-((diethoxyphosphoryl)difluoromethyl)-7-(4,4,5,5-tetramethyl-1,3,2-dioxaborolan-2-yl)benzo[b]thiophene-5-carboxylate (250 mg, 0.400 mmol), copper (II) trifluoromethanesulfonate (868 mg, 2.40 mmol), and cesium fluoride (364 mg, 2.40 mmol) in acetonitrile (2 mL) was stirred and heated to 60° C. for 2 hours. The mixture was cooled to room temperature, diluted with water, and extracted with DCM. The organic layer was separated, dried over magnesium sulfate, filtered, and concentrated. The resulting residue was purified using silica gel chromatography (eluting EtOAc in hexanes) to provide tert-butyl 3-bromo-2-((diethoxyphosphoryl)difluoromethyl)-7-fluorobenzo[b]thiophene-5-carboxylate. ¹H NMR (600 MHz, DMSO-d₆) δ 8.27 (s, 1H), 7.92 (d, J=10.1 Hz, 1H), 4.31-4.32 (m, 4H), 1.61 (s, 9H), 1.29 (t, J=7.0 Hz, 6H). MS (ESI, m/z): 517, 519 (M+H)⁺

### Step B: Synthesis of 3-bromo-2-((diethoxyphosphoryl)difluoromethyl)-7-fluorobenzo[b]thiophene-5-carboxylic acid

TFA (0.31 mL) was added to a mixture of tert-butyl 3-bromo-2-((diethoxyphosphoryl)difluoromethyl)-7-fluorobenzo[b]thiophene-5-carboxylate (34 mg, 0.066 mmol) in DCM (0.5 mL) at room temperature. The mixture was allowed to stir at room temperature for 1 hour, then concentrated in vacuo to provide 3-bromo-2-((diethoxyphosphoryl)difluoromethyl)-7-fluorobenzo[b]thiophene-5-carboxylic acid (Int-6), which was used without purification in the next step. MS (ESI, m/z): 461, 463 (M+H)⁺

### Synthesis of Intermediate Compound Int-7

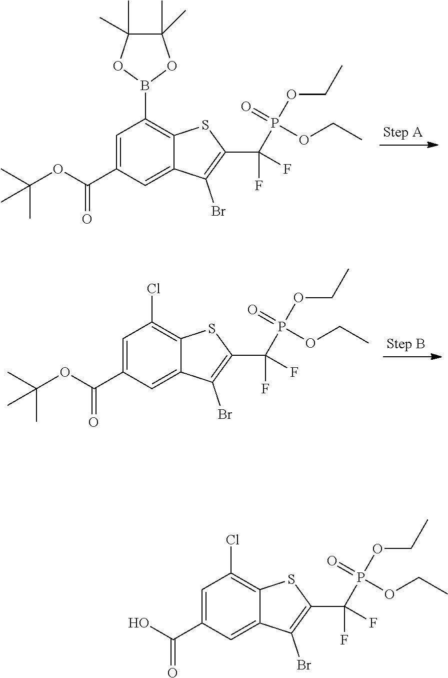

### Step A: Synthesis of tert-butyl 3-bromo-7-chloro-2-((diethoxyphosphoryl)difluoromethyl)benzo [b]thiophene-5-carboxylate

A mixture of copper (II) chloride (52 mg, 0.38 mmol) in water (0.64 mL) was added to a mixture of tert-butyl 3-bromo-2-((diethoxyphosphoryl)difluoromethyl)-7-(4,4,5,5-tetramethyl-1,3,2-dioxaborolan-2-yl)benzo[b]thiophene-5-carboxylate (80 mg, 0.13 mmol) in MeOH (0.64 mL). The mixture was stirred and heated to 100° C. for 1 hour. The mixture was cooled to room temperature, and diluted with DCM. The organic layer was separated, dried over magnesium sulfate, filtered, and concentrated in vacuo, and the resulting residue was purified using silica gel chromatography (eluting EtOAc/EtOH (3:1) in hexanes) to provide tert-butyl 3-bromo-7-chloro-2-((diethoxyphosphoryl)difluoromethyl)benzo[b]thiophene-5-carboxylate. MS (ESI, m/z): 533, 535 (M+H)⁺

### Step B: Synthesis of 3-bromo-7-chloro-2-((diethoxyphosphoryl)difluoromethyl)benzo[b]thiophene-5-carboxylic acid

TFA (0.30 mL, 3.8 mmol) was added to a mixture of tert-butyl 3-bromo-7-chloro-2-((diethoxyphosphoryl)difluoromethyl)benzo[b]thiophene-5-carboxylate (34 mg, 0.064 mmol) in DCM (0.5 mL) at room temperature. The mixture was allowed to stir at room temperature for 45 minutes. The mixture was concentrated in vacuo to provide 3-bromo-7-chloro-2-((diethoxyphosphoryl)difluoromethyl)benzo[b]thiophene-5-carboxylic acid (Int-7), which was used in the next step without purification. MS (ESI, m/z): 477, 479 (M+H)⁺

### Synthesis of Intermediate Compound Int-8a

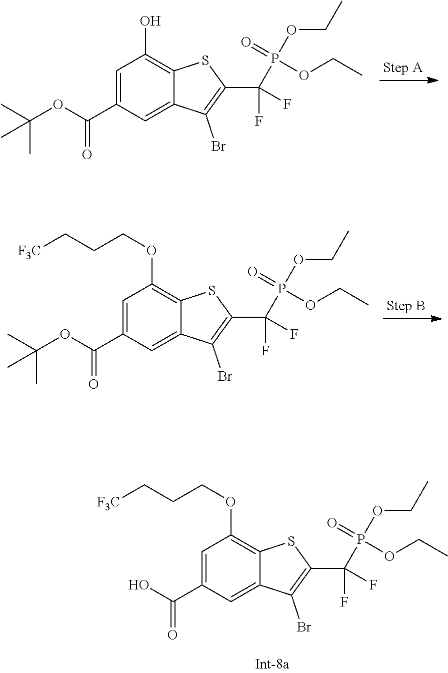

### Step A: Synthesis of tert-butyl 3-bromo-2-((diethoxyphosphoryl)difluoromethyl)-7-(4,4,4-trifluorobutoxy)benzo[b]thiophene-5-carboxylate

A mixture of tert-butyl 3-bromo-2-((diethoxyphosphoryl)difluoromethyl)-7-hydroxybenzo[b]thiophene-5-carboxylate (230 mg, 0.446 mmol), 4-bromo-1,1,1-trifluorobutane (170 mg, 0.893 mmol), and potassium carbonate (93 mg, 0.67 mmol) in acetonitrile (3 mL) was stirred and heated at 50° C. for 24 hours. The mixture was cooled to room temperature, and diluted with DCM (20 mL), then filtered through Celite, washing with DCM. The filtrate was concentrated in vacuo, and the resulting residue was purified using silica gel chromatography (eluting ethyl acetate in hexanes) to provide tert-butyl 3-bromo-2-((diethoxyphosphoryl)difluoromethyl)-7-(4,4,4-trifluorobutoxy)benzo[b]thiophene-5-carboxylate. MS (ESI, m/z): 625, 627 (M+H)⁺

### Step B: Synthesis of 3-bromo-2-((diethoxyphosphoryl)difluoromethyl)-7-(4,4,4-trifluorobutoxy)benzo[b]thiophene-5-carboxylic acid

TFA (2.0 mL, 26 mmol) was added to a mixture of tert-butyl 3-bromo-2-((diethoxyphosphoryl)difluoromethyl)-7-(4,4,4-trifluorobutoxy)benzo[b]thiophene-5-carboxylate (230 mg, 0.368 mmol) in DCM (5 mL) at room temperature. The mixture was allowed to stir at room temperature for 1 hour. The mixture was concentrated in vacuo to provide 3-bromo-2-((diethoxyphosphoryl)difluoromethyl)-7-(4,4,4-trifluorobutoxy)benzo[b]thiophene-5-carboxylic acid (Int-8a), which was used without purification in the next step. ¹H NMR (300 MHz, CDCl₃) δ 8.32 (s, 1H), 7.53 (s, 1H), 4.45-4.22 (m, 6H), 2.49-2.28 (m, 2H), 2.26-2.11 (m, 2H), 1.40 (t, J=7.1 Hz, 6H). MS (ESI, m/z): 569, 571 (M+H)⁺

The following intermediate compound was made using the methods described above and substituting the appropriate reactants and/or reagents.

| Intermediate | | |
|---|---|---|
| Compound | Structure | Mass [M + H]⁺ |
| Int-8b | 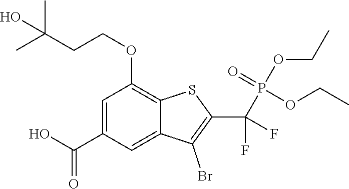 [SMILES: CCOP(=O)(OCC)C(F)(F)c1sc2c(OCCC(C)(C)O)cc(C(=O)O)cc2c1Br] | 567, 569 [M + Na]⁺ |
| Int-8c | 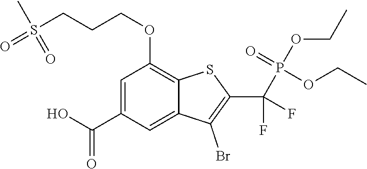 [SMILES: CCOP(=O)(OCC)C(F)(F)c1sc2c(OCCCS(C)(=O)=O)cc(C(=O)O)cc2c1Br] | 579, 581 |
| Int-8d | 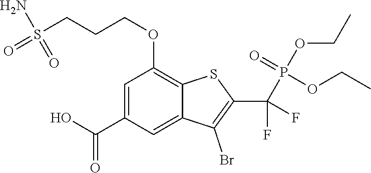 [SMILES: CCOP(=O)(OCC)C(F)(F)c1sc2c(OCCCS(N)(=O)=O)cc(C(=O)O)cc2c1Br] | 580, 582 |
| Int-8e | 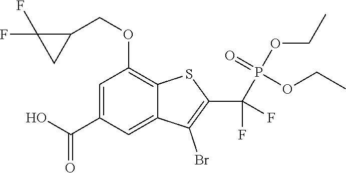 [SMILES: CCOP(=O)(OCC)C(F)(F)c1sc2c(OCC3CC3(F)F)cc(C(=O)O)cc2c1Br] | 547, 549 [M − H]⁻ |
| Int-8f | 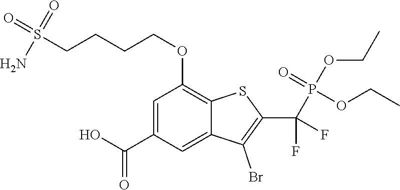 [SMILES: CCOP(=O)(OCC)C(F)(F)c1sc2c(OCCCCS(N)(=O)=O)cc(C(=O)O)cc2c1Br] | 592, 594 [M − H]⁻ |
| Int-8g ^{a} | 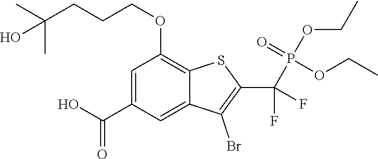 [SMILES: CCOP(=O)(OCC)C(F)(F)c1sc2c(OCCCC(C)(C)O)cc(C(=O)O)cc2c1Br] | 557, 559 [M − H]⁻ |
| Int-8h | 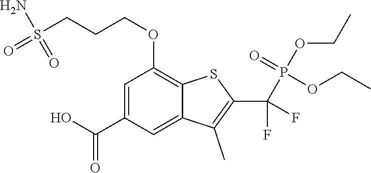 [SMILES: CCOP(=O)(OCC)C(F)(F)c1sc2c(OCCCS(N)(=O)=O)cc(C(=O)O)cc2c1C] | 516 |
| Int-8i | 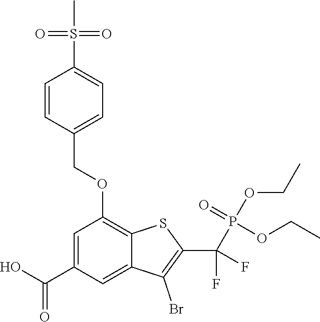 [SMILES: CCOP(=O)(OCC)C(F)(F)c1sc2c(OCc3ccc(S(C)(=O)=O)cc3)cc(C(=O)O)cc2c1Br] | 627, 629 |

^{a} Step 2: TFA replaced with Zn(II)Br₂ as acid source

### Synthesis of Intermediate Compound Int-9

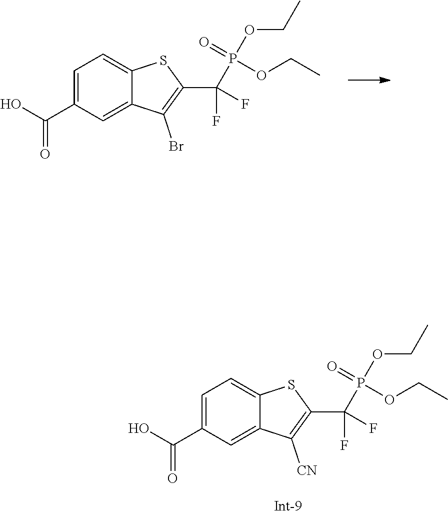 [SMILES: CCOP(=O)(OCC)C(F)(F)c1sc2ccc(C(=O)O)cc2c1Br ; CCOP(=O)(OCC)C(F)(F)c1sc2ccc(C(=O)O)cc2c1C#N]

Dicyanozinc (106 mg, 0.903 mmol) was added to a mixture of compound Int-1 (200 mg, 0.451 mmol), and Pd(Ph₃P)₄ (521 mg, 0.451 mmol) in DMF (2 mL) at room temperature under an argon atmosphere. The resulting reaction was heated to 100° C. for 1 hour. The mixture was cooled to room temperature, and directly purified using reverse phase HPLC (eluting acetonitrile in water with TFA modifier) to provide 3-cyano-2-((diethoxyphosphoryl)difluoromethyl)benzo-[b]thiophene-5-carboxylic acid (Int-9). ¹H NMR: (300 MHz, CD₃CN) δ 8.52 (br s, 1H), 8.22 (br s, 1H), 7.97 (br s, 1H), 4.38-4.17 (m, 4H), 1.30 (t, J=7.1 Hz, 6H). MS (ESI, m/z): 388 (M−H)⁻

### Synthesis of Intermediate Compound Int-10

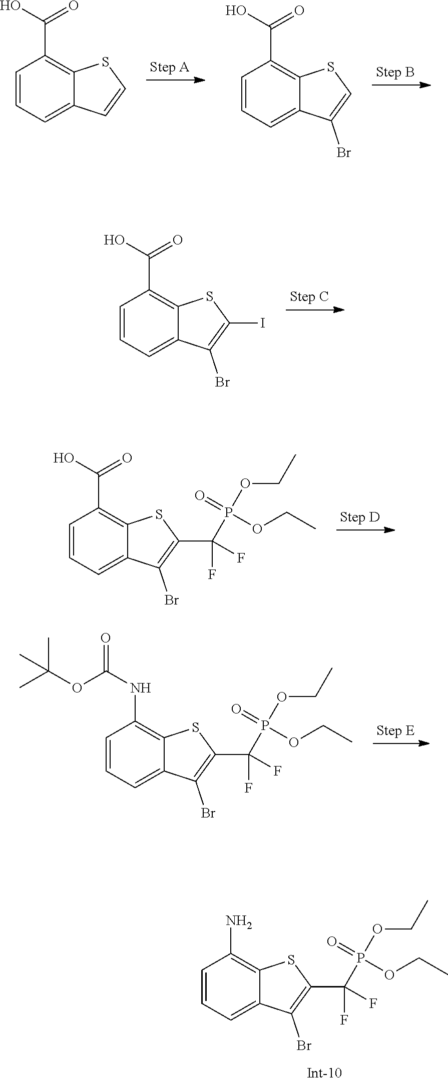 [SMILES: O=C(O)c1cccc2ccsc12 ; O=C(O)c1cccc2c(Br)csc12 ; O=C(O)c1cccc2c(Br)c(I)sc12 ; CCOP(=O)(OCC)C(F)(F)c1sc2c(C(=O)O)cccc2c1Br ; CCOP(=O)(OCC)C(F)(F)c1sc2c(NC(=O)OC(C)(C)C)cccc2c1Br ; CCOP(=O)(OCC)C(F)(F)c1sc2c(N)cccc2c1Br]

### Step A: Synthesis of 3-bromobenzo[b]thiophene-7-carboxylic acid

NBS (1.05 g, 5.89 mmol) was added to a mixture of benzo[b]thiophene-7-carboxylic acid (1.00 g, 5.61 mmol) in dichloromethane (25 mL) at room temperature. The mixture was allowed to stir at room temperature for 18 hours. The reaction mixture was concentrated in vacuo, and the resulting residue was purified using silica gel chromatography (eluting [10% methanol in ethyl acetate]in hexanes) to provide 3-bromobenzo[b]thiophene-7-carboxylic acid. MS (ESI, m/z): 257, 259 (M+H)⁺

### Step B: Synthesis of 3-bromo-2-iodobenzo[b]thiophene-7-carboxylic acid

LDA (1.0 M in THF/hexanes, 6.2 mL, 6.2 mmol) was added to a mixture of 3-bromobenzo[b]thiophene-7-carboxylic acid (535 mg, 2.08 mmol) in THF (15 mL) at −78° C. The mixture was allowed to stir at −78° C. for 30 minutes. A solution of iodine (581 mg, 2.29 mmol) in THF (3 mL) was added to the mixture at −78° C. The mixture was then warmed to room temperature, and stirred for 1 hour. The reaction mixture was quenched with saturated aqueous ammonium chloride (10 mL), then diluted with ethyl acetate (500 mL). Additional water (50 mL), and 1N HCl (12 mL) were added to the mixture. The organic layer was separated, washed with brine (25 mL), dried over magnesium sulfate, filtered, and concentrated to provide 3-bromo-2-iodobenzo[b]thiophene-7-carboxylic acid, which was used without purification in the next step. ¹H NMR (500 MHz, DMSO-d₆) δ 8.06 (d, J=7.2 Hz, 1H), 7.98 (d, J=8.2 Hz, 1H), 7.63 (t, J=7.6 Hz, 1H).

### Step C: Synthesis of 3-bromo-2-((diethoxyphosphoryl)difluoromethyl)benzo[b]thiophene-7-carboxylic acid

A solution of ((diethoxyphosphoryl)difluoromethyl)zinc(II) bromide (1.0 M in THF, 12.3 mL, 12.3 mmol) was added to a mixture of 3-bromo-2-iodobenzo[b]thiophene-7-carboxylic acid (784 mg, 2.05 mmol), and copper (I) bromide (587 mg, 4.09 mmol) in DMF (10 mL) at 25° C. under an argon atmosphere. The reaction mixture was stirred under an argon atmosphere for 5 days. The reaction mixture was quenched by the addition of saturated aqueous ammonium chloride (50 mL), then diluted with ethyl acetate (1000 mL). 1N HCl (50 mL) was added to the mixture and stirred vigorously. The organic layer was separated and washed with additional water (4×50 mL), then brine (50 mL), dried over magnesium sulfate, filtered, and concentrated in vacuo, and the resulting residue was purified using silica gel chromatography (eluting methanol in dichloromethane) to provide 3-bromo-2-((diethoxyphosphoryl)difluoromethyl)benzo [b]thiophene-7-carboxylic acid, which was used without purification in the next step. MS (ESI, m/z): 441, 443 (M−H)⁻

### Step D: Synthesis of tert-butyl (3-bromo-2 ((diethoxyphosphoryl)difluoromethyl) benzo[b]thiophen-7-yl)carbamate

A mixture of 3-bromo-2-((diethoxyphosphoryl)difluoromethyl)benzo[b]thiophene-7-carboxylic acid (907 mg, 2.05 mmol), diphenylphosphoryl azide (0.53 mL, 2.4 mmol), tert-butanol (2.0 mL, 20 mmol), and Hünig's base (0.47 mL, 2.7 mmol) in toluene (10 mL) was stirred and heated to 90° C. for 5 hours. The reaction mixture was cooled to room temperature, and concentrated in vacuo, and the resulting residue was directly purified using silica gel chromatography (eluting ethyl acetate in hexanes) to provide tert-butyl (3-bromo-2-((diethoxyphosphoryl)difluoromethyl)benzo[b]thiophen-7-yl)carbamate. ¹H NMR (500 MHz, DMSO-d₆) δ 9.65 (s, 1H), 7.72 (d, J=7.7 Hz, 1H), 7.62-7.54 (m, 2H), 4.29-4.18 (m, 4H), 1.50 (s, 9H), 1.29 (t, J=7.0 Hz, 6H). MS (ESI, m/z): 536, 538 (M+Na)⁺

### Step E: Synthesis of diethyl ((7-amino-3-bromobenzo[b]thiophen-2-yl)difluoromethyl)phosphonate

TFA (3.7 mL, 48 mmol) was added to a mixture of tert-butyl (3-bromo-2-((diethoxyphosphoryl)difluoromethyl)benzo[b]thiophen-7-yl)carbamate (490 mg, 0.953 mmol) in dichloromethane (10 mL) at room temperature. The mixture was allowed to stir at room temperature for 2 hours. The reaction mixture was concentrated in vacuo, and the resulting residue was taken up in DCM, and passed through a NaHCO₃resin cartridge to neutralize (washing with excess DCM). The filtrate was concentrated in vacuo to provide diethyl ((7-amino-3-bromobenzo[b]thiophen-2-yl)difluoromethyl)phosphonate (Int-10), which was used in the next step without purification. MS (ESI, m/z): 414, 416 (M+H)⁺

### Synthesis of Intermediate Compound Int-11a

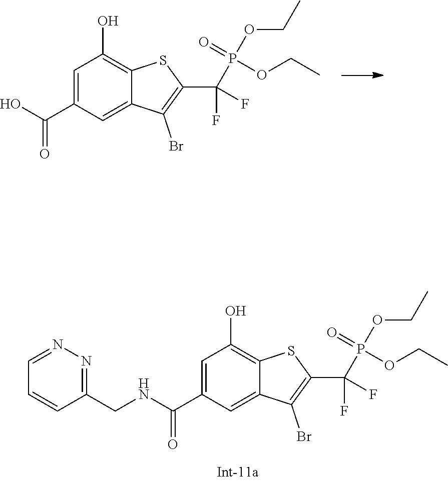 [SMILES: CCOP(=O)(OCC)C(F)(F)c1sc2c(O)cc(C(=O)O)cc2c1Br ; CCOP(=O)(OCC)C(F)(F)c1sc2c(O)cc(C(=O)NCc3cccnn3)cc2c1Br]

A mixture of compound Int-3 (267 mg, 0.582 mmol), pyridazin-3-ylmethanamine (95 mg, 0.87 mmol), HATU (266 mg, 0.699 mmol), and Hünig's base (305 μl, 1.75 mmol) in DMF (3 mL). The mixture was allowed to stir at room temperature for 30 minutes. The mixture was diluted with water and extracted with DCM. The organic layer was separated, dried over magnesium sulfate, filtered, and concentrated in vacuo, and the resulting residue was purified using silica gel chromatography (eluting [3:1 ethyl acetate:ethanol]in hexanes) to provide diethyl ((3-bromo-7-hydroxy-5-((pyridazin-3-ylmethyl)carbamoyl)benzo[b]thiophen-2-yl)difluoromethyl)phosphonate (Int-11a). MS (ESI, m/z): 550, 552 (M+H)⁺

The following intermediate compounds were made using the methods described in the Example immediately above, and substituting the appropriate reactants and/or reagents.

| Intermediate | | Mass |
|---|---|---|
| Compound | Structure | [M + H]⁺ |
| Int-11b | 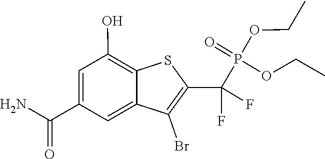 [SMILES: CCOP(=O)(OCC)C(F)(F)c1sc2c(O)cc(C(N)=O)cc2c1Br] | 458, 460 [M + H]⁺ |
| Int-11c | 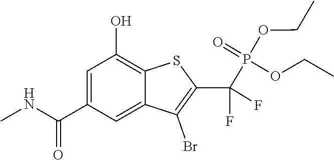 [SMILES: CCOP(=O)(OCC)C(F)(F)c1sc2c(O)cc(C(=O)NC)cc2c1Br] | 472, 474 [M + H]⁺ |
| Int-11d | 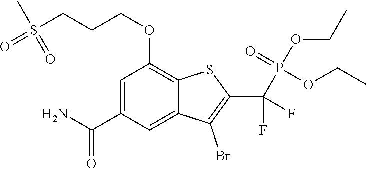 [SMILES: CCOP(=O)(OCC)C(F)(F)c1sc2c(OCCCS(C)(=O)=O)cc(C(N)=O)cc2c1Br] | 578, 580 |
| Int-11e | 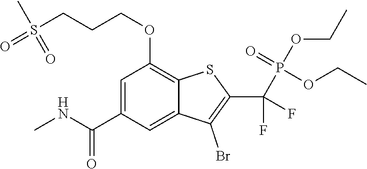 [SMILES: CCOP(=O)(OCC)C(F)(F)c1sc2c(OCCCS(C)(=O)=O)cc(C(=O)NC)cc2c1Br] | 592, 594 |
| Int-11f | 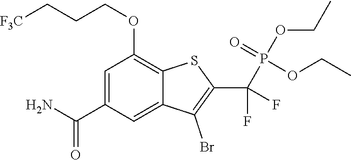 [SMILES: CCOP(=O)(OCC)C(F)(F)c1sc2c(OCCCC(F)(F)F)cc(C(N)=O)cc2c1Br] | 568, 570 |
| Int-11g ª | 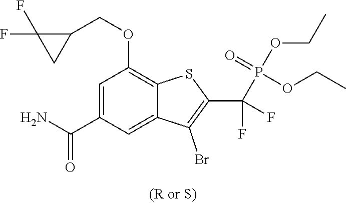 [SMILES: CCOP(=O)(OCC)C(F)(F)c1sc2c(OCC3CC3(F)F)cc(C(N)=O)cc2c1Br] | 548, 550 |
| Int-11h ^{a} | 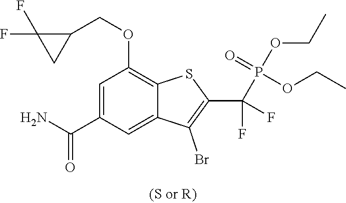 [SMILES: CCOP(=O)(OCC)C(F)(F)c1sc2c(OCC3CC3(F)F)cc(C(N)=O)cc2c1Br] | 548, 550 |
| Int-11i | 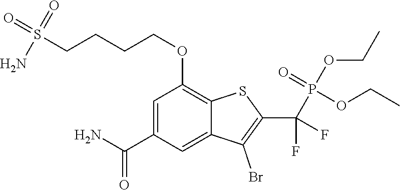 [SMILES: CCOP(=O)(OCC)C(F)(F)c1sc2c(OCCCCS(N)(=O)=O)cc(C(N)=O)cc2c1Br] | 591, 593 [M − H]⁻ |
| Int-11j | 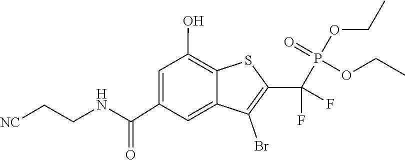 [SMILES: CCOP(=O)(OCC)C(F)(F)c1sc2c(O)cc(C(=O)NCCC#N)cc2c1Br] | 509, 511 [M − H]⁻ |
| Int-11k | 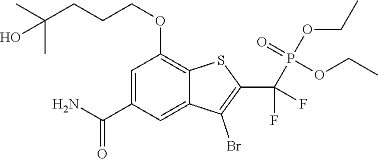 [SMILES: CCOP(=O)(OCC)C(F)(F)c1sc2c(OCCCC(C)(C)O)cc(C(N)=O)cc2c1Br] | 556, 558 [M − H]⁻ |
| Int-11l | 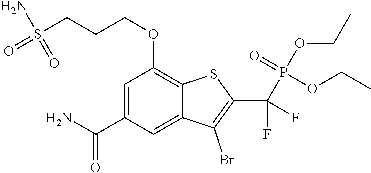 [SMILES: CCOP(=O)(OCC)C(F)(F)c1sc2c(OCCCS(N)(=O)=O)cc(C(N)=O)cc2c1Br] | 515 |
| Int-11m | 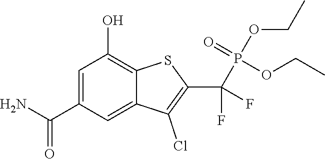 [SMILES: CCOP(=O)(OCC)C(F)(F)c1sc2c(O)cc(C(N)=O)cc2c1Cl] | 414 |
| Int-11n | 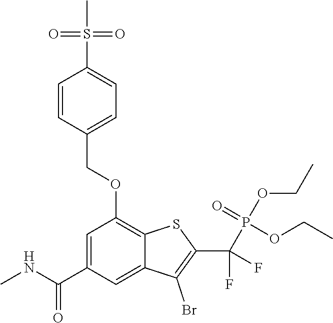 [SMILES: CCOP(=O)(OCC)C(F)(F)c1sc2c(OCc3ccc(S(C)(=O)=O)cc3)cc(C(=O)NC)cc2c1Br] | 640, 642 |

^{a} Products were separated into pure stereoisomers using the following condtions: ChiralPak IH-3; 4.5 × 50 mm, 3 μm; 40% ethanol in hexane with 0.1% diethylamine; flow rate = 1 mL/min

### Synthesis of Intermediate Compound Int-12

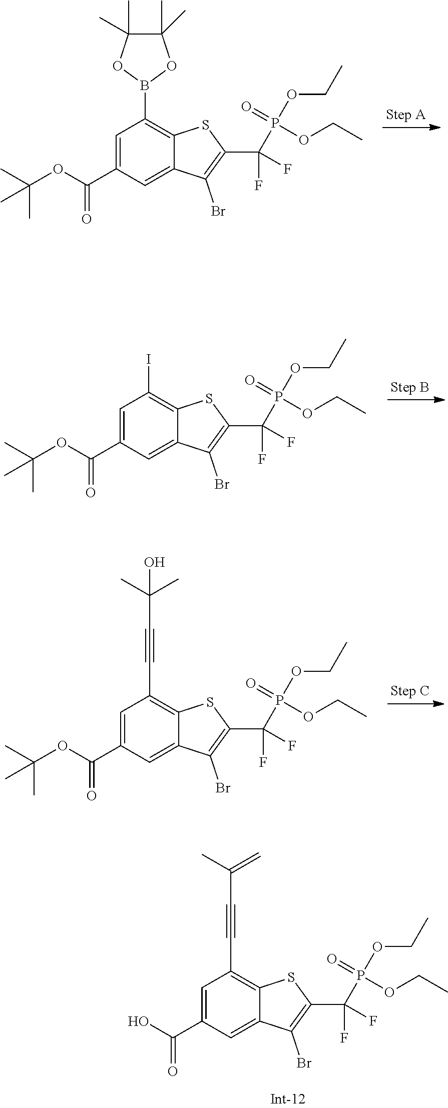

### Step A: Synthesis of tert-butyl 3-bromo-2-((diethoxyphosphoryl)difluoromethyl)-7-iodobenzo[b]thiophene-5-carboxylate

A mixture of tert-butyl 3-bromo-2-((diethoxyphosphoryl)difluoromethyl)-7-(4,4,5,5-tetramethyl-1,3,2-dioxaborolan-2-yl)benzo[b]thiophene-5-carboxylate (300 mg, 0.480 mmol), 1,10-phenanthroline (86 mg, 0.48 mmol), copper (I) iodide (46 mg, 0.24 mmol), potassium iodide (119 mg, 0.720 mmol) in MeOH (1.9 mL), and water (0.48 mL) was allowed to stir at room temperature for 15 minutes and then heated to 80° C. for 1 hour. The mixture was cooled to room temperature, diluted with water, and extracted with DCM. The organic layer was separated, dried over magnesium sulfate, filtered, and concentrated in vacuo, and the resulting residue was purified using silica gel chromatography (eluting EtOAc in hexanes) to provide tert-butyl 3-bromo-2-((diethoxyphosphoryl)difluoromethyl)-7-iodobenzo[b]thiophene-5-carboxylate. MS (ESI, m/z): 625, 627 (M+H)⁺

### Step B: Synthesis of tert-butyl 3-bromo-2-((diethoxyphosphoryl)difluoromethyl)-7-(3-hydroxy-3-methylbut-1-yn-1-yl)benzo[b]thiophene-5-carboxylate

A mixture of tert-butyl 3-bromo-2-((diethoxyphosphoryl)difluoromethyl)-7-iodobenzo[b]thiophene-5-carboxylate (30 mg, 0.048 mmol), and 2-methyl-3-butyn-2-ol (9 μl, 0.1 mmol) in THF (480 μl) was purged with nitrogen. Copper (I) iodide (4 mg, 0.02 mmol), bis(triphenylphosphine)palladium (II) dichloride (7 mg, 10 μmol), and triethylamine (13 μl, 0.096 mmol) were added to the mixture. The reaction was allowed to stir at room temperature for 3 hours. The mixture was diluted with water and extracted with DCM. The organic layer was separated, dried over magnesium sulfate, filtered, and concentrated. The resulting residue was purified using silica gel chromatography (eluting EtOAc in hexanes) to provide tert-butyl 3-bromo-2-((diethoxyphosphoryl)difluoromethyl)-7-(3-hydroxy-3-methylbut-1-yn-1-yl)benzo[b]thiophene-5-carboxylate. MS (ESI, m/z): 603, 605 (M+Na)⁺

### Step C: Synthesis of 3-bromo-2-((diethoxyphosphoryl)difluoromethyl)-7-(3-methylbut-3-en-1-yn-1-yl)benzo[b]thiophene-5-carboxylic acid

TFA (0.074 mL, 0.96 mmol) was added to a mixture of tert-butyl 3-bromo-2-((diethoxyphosphoryl)difluoromethyl)-7-(3-hydroxy-3-methylbut-1-yn-1-yl)benzo[b]thiophene-5-carboxylate (17 mg, 0.048 mmol) in DCM (0.5 mL) at room temperature. The mixture was allowed to stir at room temperature for 5 minutes. The mixture was concentrated in vacuo to provide 3-bromo-2-((diethoxyphosphoryl)difluoromethyl)-7-(3-methylbut-3-en-1-yn-1-yl)benzo[b]thiophene-5-carboxylic acid (Int-12), which was used without purification in the next step. MS (ESI, m/z): 507, 509 (M+H)⁺

### Synthesis of Intermediate Compound Int-13

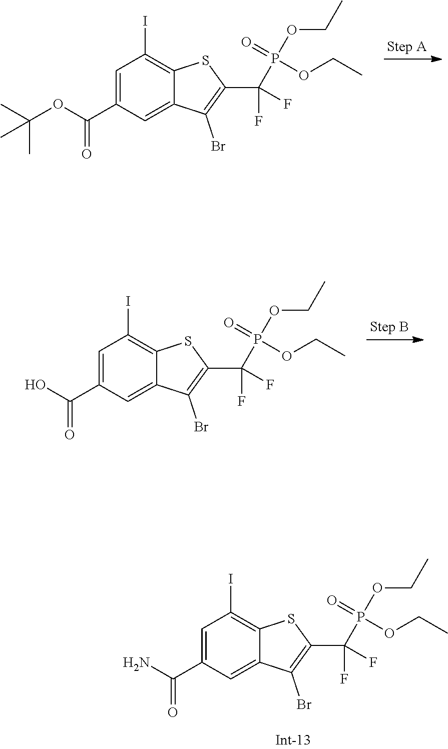 [SMILES: CCOP(=O)(OCC)C(F)(F)c1sc2c(I)cc(C(=O)OC(C)(C)C)cc2c1Br ; CCOP(=O)(OCC)C(F)(F)c1sc2c(I)cc(C(=O)O)cc2c1Br ; CCOP(=O)(OCC)C(F)(F)c1sc2c(I)cc(C(N)=O)cc2c1Br]

### Step A: Synthesis of 3-bromo-2-((diethoxyphosphoryl)difluoromethyl)-7-iodobenzo[b]thiophene-5-carboxylic acid

TFA (0.43 mL, 5.6 mmol) was added to a mixture of tert-butyl 3-bromo-2-((diethoxyphosphoryl)difluoromethyl)-7-iodobenzo[b]thiophene-5-carboxylate (140 mg, 0.224 mmol) in DCM (0.75 mL). The mixture was allowed to stir at room temperature for 1.5 hours. The mixture was concentrated in vacuo to provide 3-bromo-2-((diethoxyphosphoryl)difluoromethyl)-7-iodobenzo[b]thiophene-5-carboxylic acid, which was used without purification in the next step. MS (ESI, m/z): 569, 571 (M+H)⁺

### Step B: Synthesis of diethyl ((3-bromo-5-carbamoyl-7-iodobenzo[b]thiophen-2-yl)difluoromethyl)phosphonate

Ammonium chloride (36 mg, 0.67 mmol), HATU (128 mg, 0.336 mmol), and Hünig's base (196 μl, 1.12 mmol) were added to a mixture of 3-bromo-2-((diethoxyphosphoryl)difluoromethyl)-7-iodobenzo[b]thiophene-5-carboxylic acid in DMF (0.8 mL). The mixture was allowed to stir at room temperature for 1.5 hours. The mixture was diluted with water, and the mixture was filtered. The collected solids were dried in vacuo to provide diethyl ((3-bromo-5-carbamoyl-7-iodobenzo[b]thiophen-2-yl)difluoromethyl)phosphonate (Int-13), which was used without purification.

### Synthesis of Intermediate Compound Int-14

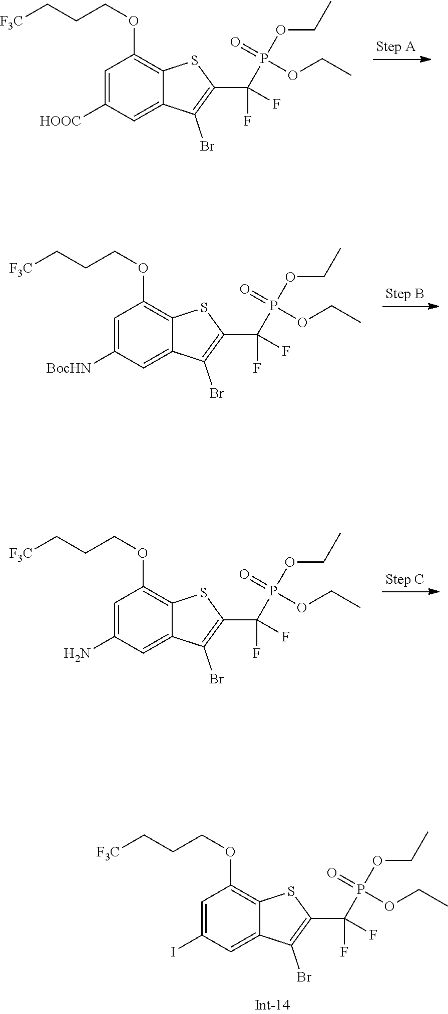 [SMILES: CCOP(=O)(OCC)C(F)(F)c1sc2c(OCCCC(F)(F)F)cc(C(=O)O)cc2c1Br ; CCOP(=O)(OCC)C(F)(F)c1sc2c(OCCCC(F)(F)F)cc(NC(=O)OC(C)(C)C)cc2c1Br ; CCOP(=O)(OCC)C(F)(F)c1sc2c(OCCCC(F)(F)F)cc(N)cc2c1Br ; CCOP(=O)(OCC)C(F)(F)c1sc2c(OCCCC(F)(F)F)cc(I)cc2c1Br]

### Step A: Synthesis of tert-butyl (3-bromo-2-((diethoxyphosphoryl)difluoromethyl)-7-(4,4,4-trifluorobutoxy)benzo[b]thiophen-5-yl)carbamate

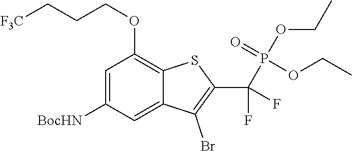 [SMILES: CCOP(=O)(OCC)C(F)(F)c1sc2c(OCCCC(F)(F)F)cc(NC(=O)OC(C)(C)C)cc2c1Br]

Triethylamine (0.269 mL, 1.93 mmol) was added to a mixture of 3-bromo-2-((diethoxyphosphoryl)difluoromethyl)-7-(4,4,4-trifluorobutoxy)benzo[b]thiophene-5-carboxylic acid (1.00 g, 1.76 mmol) in toluene (10 mL), and t-butanol (10 mL) at room temperature under an argon atmosphere. Diphenylphosphoryl azide (0.379 mL, 1.76 mmol) was added, and the resulting reaction was heated to 80° C., and allowed to stir at this temperature for 16 hours. The reaction mixture was diluted with water (50 mL), and extracted with ethyl acetate (50 mL×2). The combined organic extracts were washed with brine (100 mL×2), dried over anhydrous sodium sulfate, filtered, and concentrated in vacuo. The resulting residue was purified using silica gel column chromatography (eluting ethyl acetate in petroleum ether) to provide tert-butyl (3-bromo-2-((diethoxyphosphoryl)difluoromethyl)-7-(4,4,4-trifluorobutoxy)benzo[b]thiophen-5-yl)carbamate. ¹H NMR: (400 MHz, CDCl₃) δ 7.32-7.30 (m, 1H), 6.70 (s, 1H), 4.37-4.19 (m, 6H), 2.42-2.29 (m, 2H), 2.18-2.08 (m, 2H), 1.54 (s, 9H), 1.37 (t, J=7.1 Hz, 6H). MS (ESI, m/z): 638, 640 (M−H)⁻

### Step B: Synthesis of diethyl ((5-amino-3-bromo-7-(4,4,4-trifluorobutoxy)benzo[b]thiophen-2-yl)difluoromethyl)phosphonate

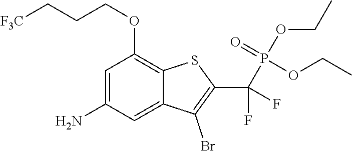 [SMILES: CCOP(=O)(OCC)C(F)(F)c1sc2c(OCCCC(F)(F)F)cc(N)cc2c1Br]

Trifluoroacetic acid (5.00 mL, 64.9 mmol) was added to a mixture of tert-butyl (3-bromo-2-((diethoxyphosphoryl)difluoromethyl)-7-(4,4,4-trifluorobutoxy)benzo[b]thiophen-5-yl)carbamate (1.20 g, 1.87 mmol) in DCM (15 mL) at −20° C. The resulting reaction was allowed to stir for 30 min at room temperature, then the reaction mixture was diluted with toluene (50 mL), and concentrated in vacuo. The resulting residue was taken up in ethyl acetate (50 mL), and the solution was adjusted to pH 8 using saturated aqueous sodium bicarbonate (100 mL), then extracted with ethyl acetate (100 mL×2). The combined organic extracts were washed with brine (100 mL×2), dried over anhydrous sodium sulfate, filtered, and concentrated in vacuo. The resulting residue was purified using a silica gel column chromatography (eluting ethyl acetate in petroleum ether) to provide diethyl ((5-amino-3-bromo-7-(4,4,4-trifluorobutoxy)benzo[b]thiophen-2-yl)difluoromethyl)phosphonate. ¹H NMR (400 MHz, CDCl₃) δ 6.89 (s, 1H), 6.39 (s, 1H), 4.37-4.21 (m, 4H), 4.16 (t, J=6.0 Hz, 2H), 2.43-2.28 (m, 2H), 2.17-2.08 (m, 2H), 1.37 (t, J=7.1 Hz, 6H). MS (ESI, m/z): 540, 542 (M+H)⁺

### Step C: Synthesis of diethyl ((3-bromo-5-iodo-7-(4,4,4-trifluorobutoxy)benzo[b]thiophen-2-yl)difluoromethyl)phosphonate

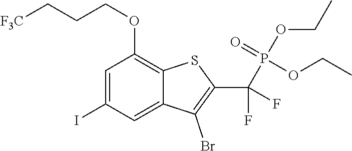 [SMILES: CCOP(=O)(OCC)C(F)(F)c1sc2c(OCCCC(F)(F)F)cc(I)cc2c1Br]

A solution of sodium nitrite (178 mg, 2.58 mmol) in water (1.2 mL) was added to a mixture of diethyl ((5-amino-3-bromo-7-(4,4,4-trifluorobutoxy)benzo[b]thiophen-2-yl)difluoromethyl)phosphonate (930 mg, 1.72 mmol) in aqueous HCl (12 M, 2.83 mL, 34 mmol) at 0° C. under an argon atmosphere. The resulting reaction was allowed to stir for 30 min at 0° C., then acetonitrile (7.7 mL), and a solution of potassium iodide (1000 mg, 6.0 mmol) in water (3.6 mL) were added to the solution. The resulting reaction was allowed to stir for 2 hours at room temperature, then was diluted with diluted with water (50 mL), and extracted with ethyl acetate (50 mL×2). The combined organic extracts were washed with brine (100 mL×2), dried over anhydrous sodium sulfate, filtered, and concentrated in vacuo. The resulting residue was purified using silica gel column chromatography (eluting ethyl acetate in petroleum ether) to provide diethyl ((3-bromo-5-iodo-7-(4,4,4-trifluorobutoxy)benzo[b]thiophen-2-yl)difluoromethyl)phosphonate (Int-14). ¹H NMR (400 MHz, CDCl₃) δ 7.90 (s, 1H), 7.12 (s, 1H), 4.38-4.23 (m, 4H), 4.21 (t, J=6.0 Hz, 2H), 2.44-2.27 (m, 2H), 2.20-2.08 (m, 2H), 1.38 (t, J=7.1 Hz, 6H). MS (ESI, m/z): 651, 653 (M+H)⁺

### Synthesis of Intermediate Compounds Int-15a and Int 15-b

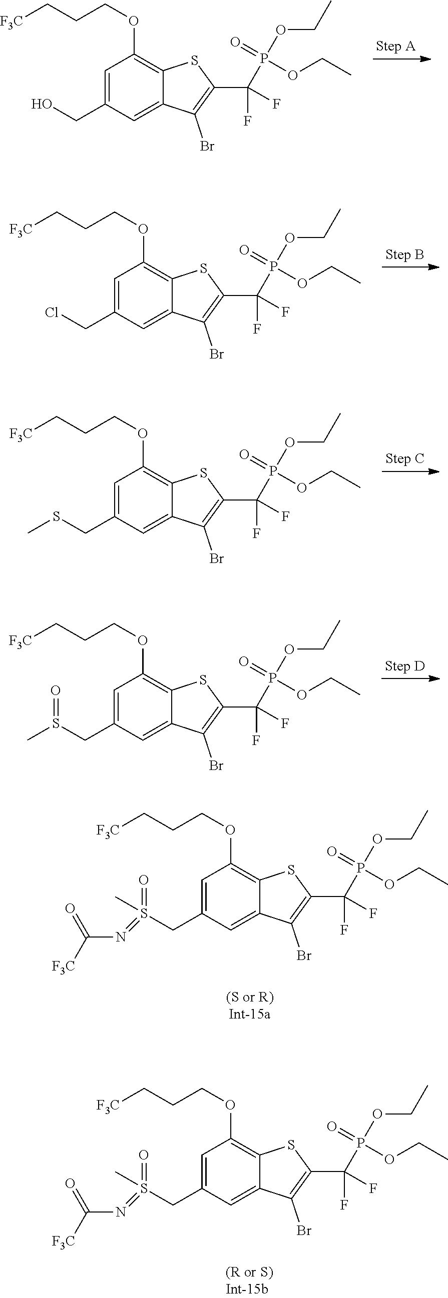 [SMILES: CCOP(=O)(OCC)C(F)(F)c1sc2c(OCCCC(F)(F)F)cc(CO)cc2c1Br ; CCOP(=O)(OCC)C(F)(F)c1sc2c(OCCCC(F)(F)F)cc(CCl)cc2c1Br ; CCOP(=O)(OCC)C(F)(F)c1sc2c(OCCCC(F)(F)F)cc(CSC)cc2c1Br ; CCOP(=O)(OCC)C(F)(F)c1sc2c(OCCCC(F)(F)F)cc(CS(C)=O)cc2c1Br ; CCOP(=O)(OCC)C(F)(F)c1sc2c(OCCCC(F)(F)F)cc(CS(C)(=O)=NC(=O)C(F)(F)F)cc2c1Br ; CCOP(=O)(OCC)C(F)(F)c1sc2c(OCCCC(F)(F)F)cc(CS(C)(=O)=NC(=O)C(F)(F)F)cc2c1Br]

### Step A: Synthesis of diethyl ((3-bromo-5-(chloromethyl)-7-(4,4,4-trifluorobutoxy)benzo[b]thiophen-2-yl)difluoromethyl)phosphonate

A solution of diethyl ((3-bromo-5-(hydroxymethyl)-7-(4,4,4-trifluorobutoxy)benzo[b]thiophen-2-yl)difluoromethyl)phosphonate (300 mg, 0.540 mmol) in sulfurous dichloride (3.00 mL, 41.1 mmol) was allowed to stir for 2 hours at 80° C. The reaction mixture was concentrated in vacuo, and the resulting residue was purified using silica gel column chromatography (eluting ethyl acetate in petroleum ether) to provide diethyl ((3-bromo-5-(chloromethyl)-7-(4,4,4-trifluorobutoxy)benzo[b]thiophen-2-yl)difluoromethyl)phosphonate. ¹H NMR (300 MHz, CDCl₃) δ 7.53 (s, 1H), 6.92 (s, 1H), 4.72 (s, 2H), 4.37-4.23 (m, 6H), 2.45-2.27 (m, 2H), 2.24-2.09 (m, 2H), 1.38 (t, J=7.1 Hz, 6H). MS (ESI, m/z): 573, 575 (M+H)⁺

### Step B: Synthesis of diethyl ((3-bromo-5-((methylthio)methyl)-7-(4,4,4-trifluorobutoxy)benzo[b]thiophen-2-yl)difluoromethyl)phosphonate

Sodium methanethiolate (71 mg, 1.0 mmol) was added to a mixture of diethyl ((3-bromo-5-(chloromethyl)-7-(4,4,4-trifluorobutoxy)benzo[b]thiophen-2-yl)difluoromethyl)phosphonate (290 mg, 0.505 mmol) in ethanol (0.6 mL) at −40° C. The resulting reaction was allowed to stir for 1 hour at 0° C. then the reaction was quenched by the addition of water (100 mL), and the resulting solution was extracted with ethyl acetate (100 mL×3). The combined organic extracts were washed with brine (100 mL), dried over anhydrous sodium sulfate, filtered, and concentrated in vacuo. The resulting residue was purified using reverse phase HPLC (eluting acetonitrile in water) to provide diethyl ((3-bromo-5-((methylthio)methyl)-7-(4,4,4-trifluorobutoxy)benzo[b]thiophen-2-yl)difluoromethyl)phosphonate. ¹H NMR (300 MHz, CDCl₃) δ 7.40 (s, 1H), 6.92 (s, 1H), 4.43-4.19 (m, 6H), 3.81 (s, 2H), 2.45-2.29 (m, 2H), 2.22-2.10 (m, 2H), 2.02 (s, 3H), 1.38 (t, J=7.1 Hz, 6H). MS (ESI, m/z): 585, 587 (M+H)⁺

### Step C: Synthesis of diethyl ((3-bromo-5-((methylsulfinyl)methyl)-7-(4,4,4-trifluorobutoxy)benzo[b]thiophen-2-yl)difluoromethyl)phosphonate

3-Chlorobenzoperoxoic acid (85 mg, 0.49 mmol) was added to a mixture of diethyl ((3-bromo-5-((methylthio)methyl)-7-(4,4,4-trifluorobutoxy)benzo[b]thiophen-2-yl)difluoromethyl)phosphonate (240 mg, 0.410 mmol) in DCM (2 mL) at 0° C. The resulting reaction was allowed to stir for 1 hour at 0° C., then was quenched by the addition of saturated aqueous NH₄Cl (30 mL), and the resulting solution was extracted with ethyl acetate (100 mL×3). The combined organic extracts were washed with brine (100 mL×3), dried over anhydrous sodium sulfate, filtered, and concentrated in vacuo. The resulting residue was purified using reverse phase HPLC (eluting acetonitrile in water) to provide diethyl ((3-bromo-5-((methylsulfinyl)methyl)-7-(4,4,4-trifluorobutoxy)benzo[b]thiophen-2-yl)difluoromethyl)phosphonate. ¹H NMR (300 MHz, CDCl₃) δ 7.42 (s, 1H), 6.86 (s, 1H), 4.37-4.23 (m, 6H), 4.10 (s, 2H), 2.53 (s, 3H), 2.41-2.28 (m, 2H), 2.22-2.09 (m, 2H), 1.38 (t, J=7.1 Hz, 6H). MS (ESI, m/z): 599, 601 (M−H)⁻

### Step D: Synthesis of diethyl ((3-bromo-5-(((S or R)-methyl-N-(2,2,2-trifluoroacetyl)sulfonimidoyl)methyl)-7-(4,4,4-trifluorobutoxy)benzo[b]thiophen-2-yl)difluoromethyl)phosphonate and diethyl ((3-bromo-5-(((R or S)-methyl-N-(2,2,2-trifluoroacetyl)sulfonimidoyl)methyl)-7-(4,4,4-trifluorobutoxy)benzo[b]thiophen-2-yl)difluoromethyl)phosphonate

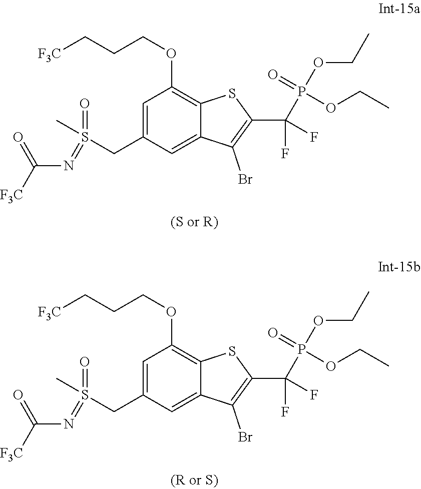 [SMILES: CCOP(=O)(OCC)C(F)(F)c1sc2c(OCCCC(F)(F)F)cc(CS(C)(=O)=NC(=O)C(F)(F)F)cc2c1Br ; CCOP(=O)(OCC)C(F)(F)c1sc2c(OCCCC(F)(F)F)cc(CS(C)(=O)=NC(=O)C(F)(F)F)cc2c1Br]

A mixture of diethyl ((3-bromo-5-((methylsulfinyl)methyl)-7-(4,4,4-trifluorobutoxy)benzo[b]thiophen-2-yl)difluoromethyl)phosphonate (160 mg, 0.266 mmol), rhodium(II) acetate dimer (3 mg, 7 μmol), 2,2,2-trifluoroacetamide (60 mg, 0.53 mmol), magnesium oxide (44 mg, 1.1 mmol), and phenyl prop-1-en-2-yloxyethanecarboperoxoyl iodide (128 mg, 0.399 mmol) in DCM (3 mL) was allowed to stir for 4 hours at room temperature. The reaction mixture was then concentrated in vacuo, and the resulting residue was purified using silica gel column chromatography (eluting ethyl acetate in petroleum ether) to provide the product as a mixture of stereoisomers, which were separated using Chiral-HPLC (Chiralpak IF, 20×250 mm, 5 μm; eluting 15% ethanol in hexane (with 0.5% 2 M NH₃-MeOH); Flow rate: 20 mL/min) to provide:

Peak 1 (10.11 minute): diethyl ((3-bromo-5-(((S or R)-methyl-N-(2,2,2-trifluoroacetyl)sulfonimidoyl)methyl)-7-(4,4,4-trifluorobutoxy)benzo[b]thiophen-2-yl)difluoromethyl)phosphonate (Int-15a). ¹H NMR (300 MHz, CDCl₃) δ 7.53 (s, 1H), 6.95 (s, 1H), 4.89 (s, 2H), 4.40-4.20 (m, 6H), 3.20 (s, 3H), 2.50-2.26 (m, 2H), 2.24-2.11 (m, 2H), 1.40 (t, J=7.1 Hz, 6H). MS (ESI, m/z): 710, 712 (M−H)⁻Peak 2 (12.74 minutes): diethyl ((3-bromo-5-(((R or S)-methyl-N-(2,2,2-trifluoroacetyl)sulfonimidoyl)methyl)-7-(4,4,4-trifluorobutoxy) benzo[b]thiophen-2-yl)difluoromethyl)phosphonate (Int-15b). ¹H NMR (300 MHz, CDCl₃) δ 7.53 (s, 1H), 6.95 (s, 1H), 4.89 (s, 2H), 4.45-4.17 (m, 6H), 3.20 (s, 3H), 2.46-2.28 (m, 2H), 2.26-2.09 (m, 2H), 1.46-1.33 (m, 6H). MS (ESI, m/z): 710, 712 (M−H)⁻

The following intermediate compounds were made using the methods described in the Example immediately above, and substituting the appropriate reactants and/or reagents.

| Intermediate | | Mass |
|---|---|---|
| Compound | Structure | [M + H]⁺ |
| Int-15c | 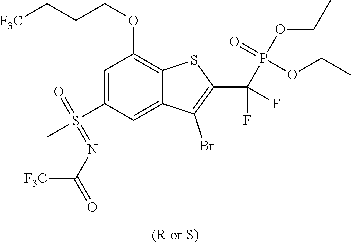 [SMILES: CCOP(=O)(OCC)C(F)(F)c1sc2c(OCCCC(F)(F)F)cc(S(C)(=O)=NC(=O)C(F)(F)F)cc2c1Br] | 698, 700 |
| Int-15d | 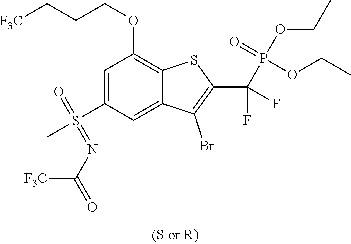 [SMILES: CCOP(=O)(OCC)C(F)(F)c1sc2c(OCCCC(F)(F)F)cc(S(C)(=O)=NC(=O)C(F)(F)F)cc2c1Br] | 698, 700 |

### Synthesis of Intermediate Compound Int-16a

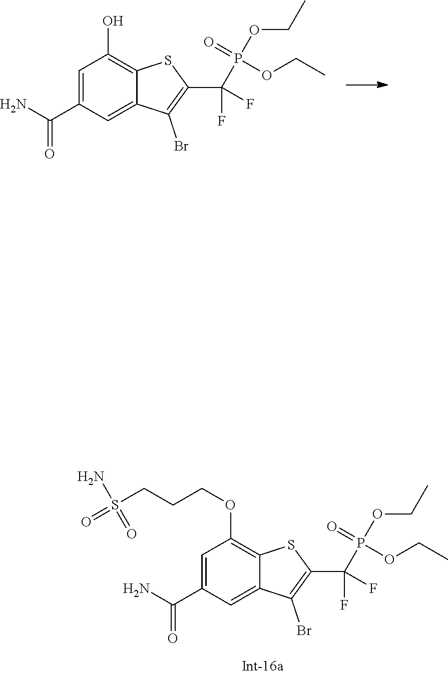 [SMILES: CCOP(=O)(OCC)C(F)(F)c1sc2c(O)cc(C(N)=O)cc2c1Br ; CCOP(=O)(OCC)C(F)(F)c1sc2c(OCCCS(N)(=O)=O)cc(C(N)=O)cc2c1Br]

A mixture of diethyl ((3-bromo-5-carbamoyl-7-hydroxybenzo[b]thiophen-2-yl)difluoromethyl)phosphonate (100 mg, 0.22 mmol), potassium carbonate (45 mg, 0.33 mmol), and 3-bromopropane-1-sulfonamide (88 mg, 0.44 mmol) in DMF (1.5 mL) was stirred at room temperature for 16 hours. The reaction mixture was then diluted with water (0.2 mL), and the solution was directly using reverse phase HPLC (eluting acetonitrile in water) to provide diethyl ((3-bromo-5-carbamoyl-7-(3-sulfamoylpropoxy)benzo[b]thiophen-2-yl)difluoromethyl)phosphonate (Int-16a). ¹H NMR (300 MHz, CD₃CN) δ 7.99 (s, 1H), 7.49 (s, 1H), 4.42 (t, J=6.1 Hz, 2H), 4.35-4.16 (m, 4H), 3.35-3.23 (m, 2H), 2.41-2.26 (m, 2H), 1.32 (t, J=7.1, 6H). MS (ESI, m/z): 579, 581 (M+H)⁺

The following intermediate compounds were made using the methods described in the Example immediately above, and substituting the appropriate reactants and/or reagents.

| Intermediate | | Mass |
|---|---|---|
| Compound | Structure | [M + H]⁺ |
| Int-16b | 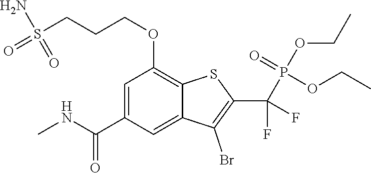 [SMILES: CCOP(=O)(OCC)C(F)(F)c1sc2c(OCCCS(N)(=O)=O)cc(C(=O)NC)cc2c1Br] | 593, 595 |
| Int-16c | 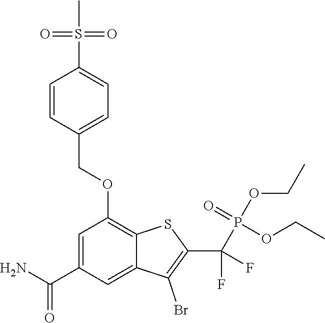 [SMILES: CCOP(=O)(OCC)C(F)(F)c1sc2c(OCc3ccc(S(C)(=O)=O)cc3)cc(C(N)=O)cc2c1Br] | 624, 626 [M − H]⁻ |

### Synthesis of Intermediate Compound Int-17

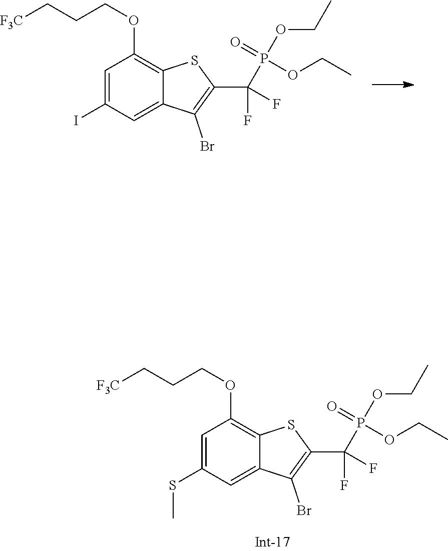 [SMILES: CCOP(=O)(OCC)C(F)(F)c1sc2c(OCCCC(F)(F)F)cc(I)cc2c1Br ; CCOP(=O)(OCC)C(F)(F)c1sc2c(OCCCC(F)(F)F)cc(SC)cc2c1Br]

A mixture of diethyl ((3-bromo-5-iodo-7-(4,4,4-trifluorobutoxy)benzo[b]thiophen-2-yl)difluoromethyl)phosphonate (250 mg, 0.384 mmol), XantPhos (13 mg, 0.023 mmol), diisopropylethylamine (0.067 mL, 0.38 mmol), tris(dibenzylideneacetone)dipalladium-chloroform adduct (14 mg, 0.013 mmol), and methyl mercaptan (10% in propylene glycol, 240 mg, 0.499 mmol) in 1,4-dioxane (2.5 mL) was stirred and heated for 2 hours at 90° C. The mixture was cooled to room temperature, quenched with saturated aqueous NH₄Cl (0.5 mL), and directly purified using reverse phase HPLC (eluting acetonitrile in water) to provide diethyl ((3-bromo-5-(methylthio)-7-(4,4,4-trifluorobutoxy)benzo[b]thiophen-2-yl)difluoromethyl)phosphonate (Int-17). ¹H NMR (400 MHz, CDCl₃) δ 7.35 (s, 1H), 6.79 (s, 1H), 4.35-4.25 (m, 4H), 4.21 (t, J=5.9 Hz, 2H), 2.59 (s, 3H), 2.42-2.30 (m, 2H), 2.18-2.10 (m, 2H), 1.38 (t, J=7.1 Hz, 6H). MS (ESI, m/z): 571, 573 (M+H)⁺

### Synthesis of Intermediate Compound Int-18

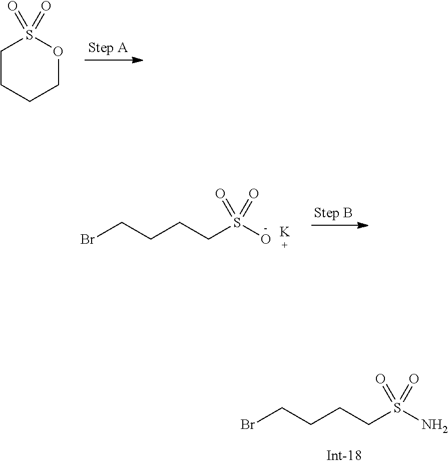

### Step A: Synthesis of potassium 4-bromobutane-1-sulfonate

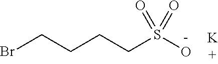

1,2-Oxathiane 2,2-dioxide (5.00 g, 36.7 mmol) was added to a mixture of potassium bromide (4.37 g, 36.7 mmol) in water (8 mL) at 25° C. under an argon atmosphere. The mixture was stirred and heated for 1 hour at 60° C. The mixture was cooled to room temperature and diluted with ethanol (150 mL). The suspension was filtered and the collected solids were collected and dried under reduced pressure to provide potassium 4-bromobutane-1-sulfonate. ¹H NMR (300 MHz, D₂O) δ 3.54 (t, J=6.4 Hz, 2H), 3.00-2.89 (m, 2H), 2.08-1.81 (m, 4H).

### Step B: Synthesis of 4-bromobutane-1-sulfonamide

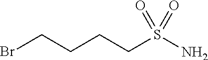

Water (0.05 mL) was added to a mixture of potassium 4-bromobutane-1-sulfonate (1.00 g, 3.92 mmol), and phosphorus pentachloride (1.06 g, 5.09 mmol). The mixture was stirred and heated for 10 min at 70° C. The mixture was cooled to room temperature and quenched with ice-water (50 mL). The mixture was extracted with diethyl ether (50 mL×2). The combined organic layers were washed with brine (50 mL), dried over anhydrous sodium sulfate, filtered, and concentrated to a total volume of 20 mL. The mixture was cooled to 0° C. and diluted with ammonia (28% in water, 3.0 mL, 3.9 mmol). The mixture was allowed to stir for 20 min at 0° C. The reaction was quenched with brine (50 mL), and extracted with ethyl ether (50 mL×2). The combined organic extracts were dried over anhydrous sodium sulfate, filtered, and concentrated in vacuo to provide 4-bromobutane-1-sulfonamide (Int-18). ¹H NMR (300 MHz, CDCl₃) δ 4.68 (s, 2H), 3.50-3.39 (m, 2H), 3.22-3.11 (m, 2H), 2.13-1.97 (m, 4H).

### Synthesis of Intermediate Compound Int-19

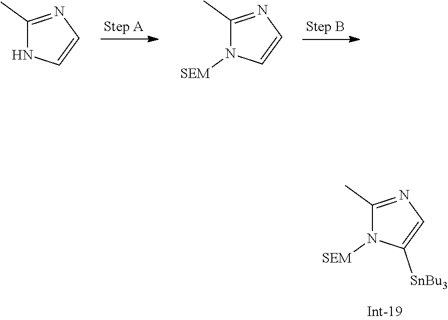

### Step A: Synthesis of 2-methyl-1-((2-(trimethylsilyl)ethoxy)methyl)-1H-imidazole

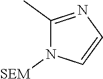

Sodium hydride (60% in mineral oil dispersion, 1.07 g, 27 mmol) was added to a mixture of 2-methyl-1H-imidazole (2.00 g, 24.4 mmol) in THF (80 mL) at 0° C. under an argon atmosphere. The resulting reaction was allowed to stir for 0.5 hours at 0° C. 2-(trimethylsilyl)ethoxymethyl chloride (4.32 mL, 24.4 mmol) was then added, and the reaction mixture was warmed to room temperature and stirred at this temperature for 1 hour. The reaction mixture was quenched with water (500 mL), and extracted with ethyl acetate (500 mL×3). The combined organic extracts were washed with brine (200 mL×2), dried over anhydrous sodium sulfate, filtered, and concentrated in vacuo. The resulting residue was purified using silica gel chromatography (eluting methanol in dichloromethane) to provide 2-methyl-1-((2-(trimethylsilyl)ethoxy)methyl)-1H-imidazole. ¹H NMR (300 MHz, CDCl₃) δ 6.87 (s, 2H), 5.15 (s, 2H), 3.51-3.37 (m, 2H), 2.40 (s, 3H), 0.90-0.82 (m, 2H), −0.05 (s, 9H).

### Step B: Synthesis of 2-methyl-5-(tributylstannyl)-1-((2-(trimethylsilyl)ethoxy)methyl)-1H-imidazole

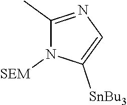

n-Butyllithium (2.5 M in THF, 1.88 mL, 4.7 mmol) was added to a mixture of 2-methyl-1-((2-(trimethylsilyl)ethoxy)methyl)-1H-imidazole (1.00 g, 4.71 mmol) in ethyl ether (25 mL) at 0° C. under an argon atmosphere. The mixture was allowed to stir for 1 hour at 25° C. Tributyltin chloride (1.53 g, 4.71 mmol) was added to the mixture at 0° C. The mixture was allowed to stir for 2 hours at 25° C. The reaction was quenched with water (500 mL), and extracted with ethyl ether (400 mL×3). The combined organic extracts were washed with brine (300 mL×3), dried over anhydrous sodium sulfate, filtered, and concentrated in vacuo. The resulting residue was purified using silica gel chromatography (eluting ethyl acetate in petroleum ether) to provide 2-methyl-5-(tributylstannyl)-1-((2-(trimethylsilyl)ethoxy)methyl)-1H-imidazole (Int-19). ¹H NMR (300 MHz, CDCl₃) δ 6.89 (s, 1H), 5.13 (s, 2H), 3.49-3.32 (m, 2H), 2.47 (s, 3H), 1.57-1.43 (m, 6H), 1.37-1.28 (m, 6H), 1.11-1.00 (m, 6H), 0.92-0.85 (m, 11H), —0.01 (s, 9H).

### Synthesis of Intermediate Compound Int-20

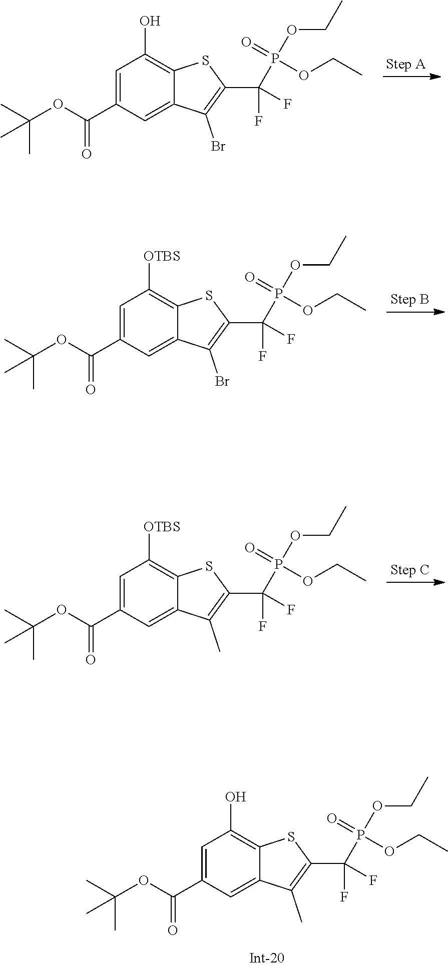

### Step A: Synthesis of tert-butyl 3-bromo-7-((tert-butyldimethylsilyl)oxy)-2-((diethoxyphosphoryl)difluoromethyl)benzo[b]thiophene-5-carboxylate

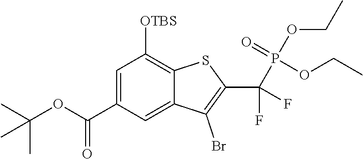 [SMILES: CCOP(=O)(OCC)C(F)(F)c1sc2c(O[Si](C)(C)C(C)(C)C)cc(C(=O)OC(C)(C)C)cc2c1Br]

Imidazole (66 mg, 0.97 mmol) was added to a mixture of tert-butyl 3-bromo-2-((diethoxyphosphoryl)difluoromethyl)-7-hydroxybenzo[b]thiophene-5-carboxylate (200 mg, 0.388 mmol), and TBS-Cl (70 mg, 0.47 mmol) in DCM (3.9 mL). The resulting reaction was allowed to stir for 18 hours at room temperature, then the reaction mixture was diluted with ethyl acetate and water. The organic layer was separated, dried over magnesium sulfate, filtered, and concentrated in vacuo, and the resulting residue was purified using silica gel column chromatography (eluting ethyl acetate in hexanes) to provide tert-butyl 3-bromo-7-((tert-butyldimethylsilyl)oxy)-2-((diethoxyphosphoryl)difluoromethyl)benzo[b]thiophene-5-carboxylate. MS (ESI, m/z): 629, 631 (M+H)⁺

### Step B: Synthesis of tert-butyl 7-((tert-butyldimethylsilyl)oxy)-2-((diethoxyphosphoryl)difluoromethyl)-3-methylbenzo[b]thiophene-5-carboxylate

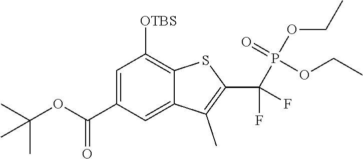 [SMILES: CCOP(=O)(OCC)C(F)(F)c1sc2c(O[Si](C)(C)C(C)(C)C)cc(C(=O)OC(C)(C)C)cc2c1C]

A mixture of tert-butyl 3-bromo-7-((tert-butyldimethylsilyl)oxy)-2-((diethoxyphosphoryl)difluoromethyl)benzo[b]thiophene-5-carboxylate (232 mg, 0.369 mmol) in 1,4-dioxane (3.7 mL) was purged with argon for 5 minutes. Trimethylboroxine (103 μl, 0.737 mmol), bis(di-tert-butyl(4-dimethylaminophenyl)phosphine)dichloropalladium (II) (52 mg, 0.074 mmol), and potassium carbonate (255 mg, 1.84 mmol) were added to the mixture. The mixture was heated in a microwave reactor at 120° C. for 6 minutes. The mixture was diluted with ethyl acetate and water. The mixture was filtered through celite and then the organic layer was separated, dried over magnesium sulfate, filtered, and concentrated in vacuo. The resulting residue was purified using silica gel chromatography (eluting ethyl acetate in hexanes) to provide tert-butyl 7-((tert-butyldimethylsilyl)oxy)-2-((diethoxyphosphoryl)difluoromethyl)-3-methylbenzo[b]thiophene-5-carboxylate. MS (ESI, m/z): 565 (M+H)⁺

### Step C: Synthesis of tert-butyl 2-((diethoxyphosphoryl)difluoromethyl)-7-hydroxy-3-methylbenzo[b]thiophene-5-carboxylate

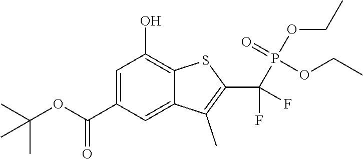 [SMILES: CCOP(=O)(OCC)C(F)(F)c1sc2c(O)cc(C(=O)OC(C)(C)C)cc2c1C]

A mixture of tert-butyl 7-((tert-butyldimethylsilyl)oxy)-2-((diethoxyphosphoryl)difluoromethyl)-3-methylbenzo[b]thiophene-5-carboxylate (157 mg, 0.278 mmol), and TBAF (556 μl, 0.556 mmol) in THF (3 mL) was stirred at room temperature for one hours. The mixture was diluted with ethyl acetate and water. The organic layer was separated, dried over magnesium sulfate, filtered, and concentrated in vacuo. The resulting residue was purified using silica gel chromatography (eluting ethyl acetate in hexanes) to provide tert-butyl 2-((diethoxyphosphoryl)difluoromethyl)-7-hydroxy-3-methylbenzo[b]thiophene-5-carboxylate (Int-20). MS (ESI, m/z): 451 (M+H)⁺

### Synthesis of Intermediate Compound Int-21

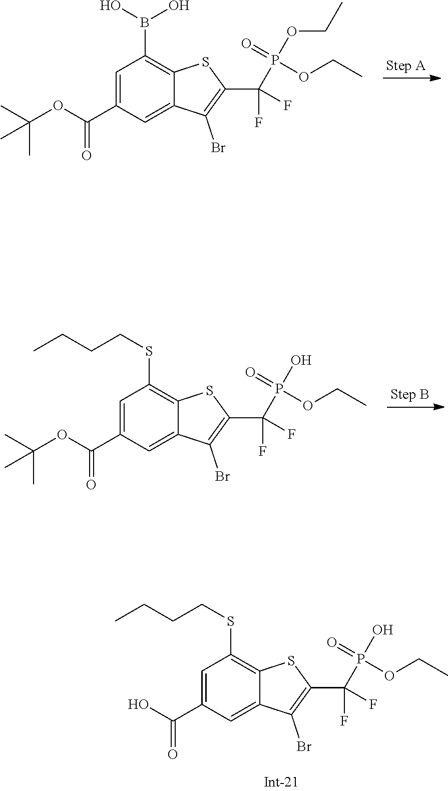

### Step A: Synthesis of tert-butyl 3-bromo-7-(butylthio)-2-((ethoxy(hydroxy)phosphoryl)difluoromethyl)benzo[b]thiophene-5-carboxylate

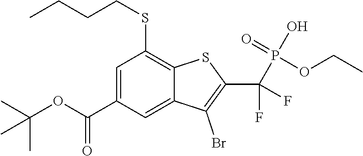 [SMILES: CCCCSc1cc(C(=O)OC(C)(C)C)cc2c(Br)c(C(F)(F)P(=O)(O)OCC)sc12]

A mixture of (3-bromo-5-(tert-butoxycarbonyl)-2-((diethoxyphosphoryl)difluoromethyl)benzo[b]thiophen-7-yl)boronic acid (30 mg, 0.055 mmol), copper(II) acetate (15 mg, 0.083 mmol), pyridine (9 mg, 0.1 mmol), butane-1-thiol (10 mg, 0.1 mmol), and 4A molecular sieves in DMF (2 mL) was stirred and heated at 100° C. for 2 hours. The mixture was directly purified using reverse phase HPLC (eluting acetonitrile in water) to provide tert-butyl 3-bromo-7-(butylthio)-2-((ethoxy(hydroxy)phosphoryl)difluoromethyl)-benzo[b]thiophene-5-carboxylate. ¹H NMR (400 MHz, DMSO-d₆) δ 8.21 (s, 1H), 7.97 (s, 1H), 3.90-3.82 (m, 2H), 3.16 (t, J=7.3 Hz, 2H), 1.65-1.53 (m, 2H), 1.60 (s, 9H), 1.47-1.36 (m, 2H), 1.11 (t, J=6.9 Hz, 3H), 0.89 (t, J=7.3 Hz, 3H). MS (ESI, m/z): 557, 559 (M−H)⁻

### Step B: Synthesis of 3-bromo-7-(butylthio)-2-((ethoxy(hydroxy)phosphoryl)difluoromethyl)benzo[b]thiophene-5-carboxylic acid

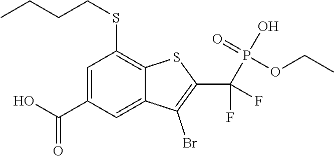 [SMILES: CCCCSc1cc(C(=O)O)cc2c(Br)c(C(F)(F)P(=O)(O)OCC)sc12]

A mixture of tert-butyl 3-bromo-7-(butylthio)-2-((ethoxy(hydroxy)phosphoryl)difluoromethyl)benzo[b]thiophene-5-carboxylate (10 mg, 0.018 mmol) in trifluoroacetic acid (0.5 mL, 6.49 mmol), and dichloromethane (0.5 mL) was stirred at 25° C. for 6 hours. The mixture was concentrated in vacuo to provide 3-bromo-7-(butylthio)-2-((ethoxy(hydroxy)phosphoryl)difluoromethyl)benzo[b]thiophene-5-carboxylic acid (Int-21), which was used without purification in the next step. MS (ESI, m/z): 501, 503 (M−H)⁻

### Synthesis of Intermediate Compound Int-22

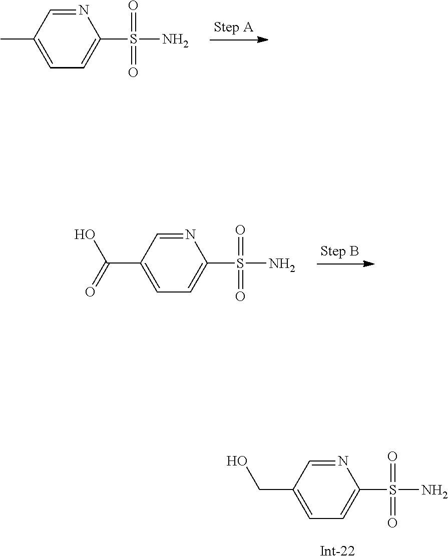

### Step A: Synthesis of 6-sulfamoylnicotinic acid

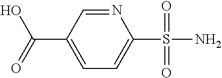 [SMILES: NS(=O)(=O)c1ccc(C(=O)O)cn1]

Sodium hydroxide (1.0 M in water, 12 mL, 12 mmol), and potassium permanganate (9.18 g, 58.1 mmol) were added to a mixture of 5-methylpyridine-2-sulfonamide (5.00 g, 29.0 mmol) in water (20 mL) at 25° C. under an argon atmosphere. The resulting reaction was heated to 100° C. for 3 hours. The mixture was cooled to 25° C. The mixture was filtered through Celite. The pH value of the filtrate was adjusted to −1 with aqueous NaHSO₄. The mixture was diluted with water (500 mL), and extracted with ethyl acetate (1500 mL×3). The combined organic extracts were washed with brine (500 mL×3), dried over anhydrous sodium sulfate, filtered, and concentrated in vacuo to provide 6-sulfamoylnicotinic acid, which was used without purification in the next step. ¹H NMR (300 MHz, DMSO-d₆) δ 13.80 (br s, 1H), 9.16 (dd, J=2.2, 0.9 Hz, 1H), 8.59-8.46 (m, 1H), 8.05 (dd, J=8.2, 0.9 Hz, 1H), 7.66 (br s, 2H). MS (ESI, m/z): 203 (M+H)⁺

### Step B: Synthesis of 5-(hydroxymethyl)pyridine-2-sulfonamide

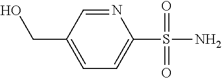 [SMILES: NS(=O)(=O)c1ccc(CO)cn1]

Borane (1.0 M in THF, 9.9 mL, 9.9 mmol) was added to a mixture of 6-sulfamoylnicotinic acid (200 mg, 0.989 mmol) in THF (2 mL) at 0° C. under an argon atmosphere. The mixture was allowed to stir for 2 hours at 25° C. The reaction was quenched with ethanol (30 mL), and concentrated in vacuo. The mixture was diluted with ethyl acetate (150 mL). The organic layer was washed with water (30 mL), and brine (100 mL). The organic layer was dried over anhydrous sodium sulfate, filtered, and concentrated in vacuo. The resulting residue was triturated with diethyl ether and filtered. The collected solids were washed with hexane (100 mL×3), and the solids were then dried to provide 5-(hydroxymethyl)pyridine-2-sulfonamide (Int-22). ¹H NMR (300 MHz, DMSO-d₆) δ 8.64 (d, J=2.0 Hz, 1H), 7.98 (dd, J=8.1, 2.1 Hz, 1H), 7.91 (d, J=8.1 Hz, 1H), 4.64 (s, 2H). MS (ESI, m/z): 189 (M+H)⁺

### Synthesis of Intermediate Compound Int-23

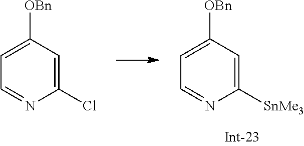

Dichlorobis(triphenylphosphine)palladium(II) (48 mg, 0.068 mmol), and hexamethylditin (0.094 mL, 0.46 mmol) were added to a mixture of 4-(benzyloxy)-2-chloropyridine (50 mg, 0.23 mmol) in 1,4-dioxane (0.5 mL) at 25° C. under an argon atmosphere. The mixture was stirred and heated for 3 hours at 90° C. The mixture was cooled to room temperature to provide 4-(benzyloxy)-2-(trimethylstannyl)pyridine (Int-23), which was used directly in the next step without workup, concentration, or purification. MS (ESI, m/z): 350 (M+H)⁺

### Synthesis of Intermediate Compound Int-24

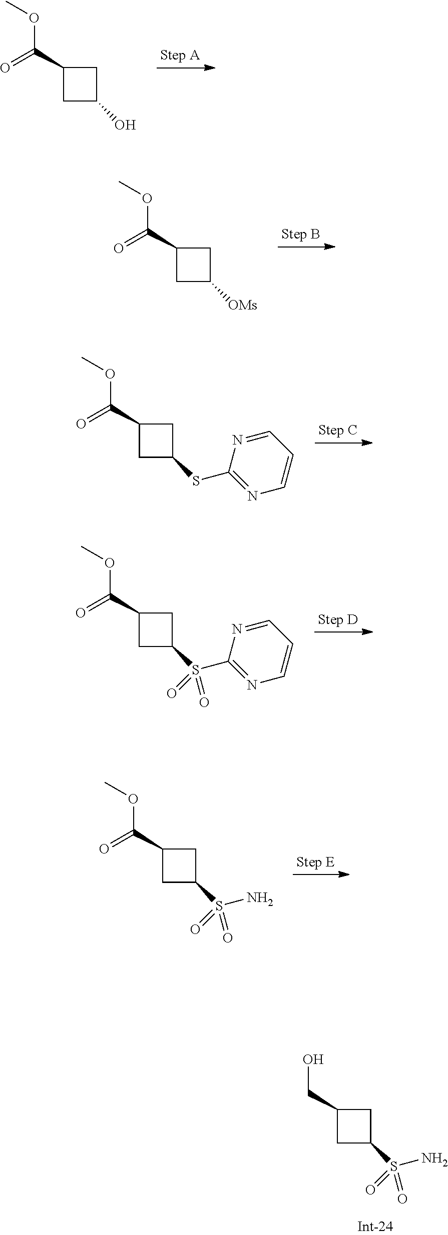

### Step A: Synthesis of methyl (trans)-3-((methylsulfonyl)oxy)cyclobutane-1-carboxylate

Methanesulfonyl chloride (5.39 mL, 69.2 mmol) was added to a mixture of methyl (trans)-3-hydroxycyclobutane-1-carboxylate (5.00 g, 38.4 mmol), and TEA (11 mL, 77 mmol) in DCM (60 mL) at 0° C. The mixture was allowed to stir for 2 hours at 25° C. The mixture was diluted with 1 M HCl (250 mL), and extracted with DCM (300 mL×3). The combined organic extracts were washed with brine (200 mL×3), dried over anhydrous sodium sulfate, filtered, and concentrated in vacuo to provide synthesis of methyl (trans)-3-((methylsulfonyl)oxy)cyclobutane-1-carboxylate, which was used in the next step without purification.

### Step B: Synthesis of methyl (cis)-3-(pyrimidin-2-ylthio)cyclobutane-1-carboxylate

A mixture of methyl (trans)-3-((methylsulfonyl)oxy)cyclobutane-1-carboxylate (8.00 g, 38.4 mmol), pyrimidine-2-thiol (12.9 g, 115 mmol), and potassium carbonate (11.2 g, 81.0 mmol) in DMF (80 mL) was stirred and heated for 2 hours at 70° C. The mixture was diluted with water (500 mL), and extracted with ethyl acetate (500 mL×3). The combined organic extracts were washed with brine (200 mL×2), dried over anhydrous sodium sulfate, filtered, and concentrated in vacuo. The resulting residue was purified using silica gel chromatography (eluting ethyl acetate in petroleum ether) to provide methyl (cis)-3-(pyrimidin-2-ylthio)cyclobutane-1-carboxylate. ¹H NMR (300 MHz, CDCl₃) δ 8.48 (d, J=4.9 Hz, 2H), 6.94 (t, J=4.9 Hz, 1H), 4.37-4.22 (m, 1H), 3.69 (s, 3H), 3.21-3.04 (m, 1H), 2.85-2.70 (m, 2H), 2.51-2.34 (m, 2H). MS (ESI, m/z): 225 (M+H)⁺

### Step C: Synthesis of methyl (cis)-3-(pyrimidin-2-ylsulfonyl)cyclobutane-1-carboxylate

mCPBA (6.77 g, 39.2 mmol) was added to a mixture of methyl (cis)-3-(pyrimidin-2-ylthio)cyclobutane-1-carboxylate (4.00 g, 17.8 mmol) in DCM (80 mL) at 25° C. under an argon atmosphere. The mixture was allowed to stir for 16 hours at 25° C. The reaction was quenched with water (500 mL), and extracted with dichloromethane (500 mL×3). The combined organic extracts were washed with brine (300 mL×3), dried over anhydrous sodium sulfate, filtered, and concentrated in vacuo. The resulting residue was purified using reverse phase HPLC (eluting acetonitrile in water) to provide methyl (cis)-3-(pyrimidin-2-ylsulfonyl)cyclobutane-1-carboxylate. ¹H NMR (300 MHz, CDCl₃) δ 8.91 (d, J=4.9 Hz, 2H), 7.57 (t, J=4.9 Hz, 1H), 4.48-4.28 (m, 1H), 3.69 (s, 3H), 3.27-3.09 (m, 1H), 3.01-2.84 (m, 2H), 2.67-2.47 (m, 2H). MS (ESI, m/z): 257 (M+H)⁺

### Step D: Synthesis of methyl (cis)-3-sulfamoylcyclobutane-1-carboxylate

Sodium methanolate (0.632 g, 11.7 mmol) was added to a mixture of methyl (cis)-3-(pyrimidin-2-ylsulfonyl)cyclobutane-1-carboxylate (3.00 g, 11.7 mmol) in MeOH (100 mL) at 25° C. under an argon atmosphere. The mixture was allowed to stir for 1 hour at 25° C. The mixture was concentrated in vacuo and The resulting residue was taken up in water (120 mL) at 25° C. under an argon atmosphere. Sodium acetate (1.92 g, 23.4 mmol), and hydroxylamine-O-sulfonic acid (1.99 g, 17.6 mmol) were added to the mixture at 25° C. The mixture was stirred and heated for 1 hour at 50° C. The mixture was directly purified using reverse phase HPLC (eluting acetonitrile in water) to provide methyl (cis)-3-sulfamoylcyclobutane-1-carboxylate. ¹H NMR (300 MHz, CD₃CN) δ 3.81-3.65 (m, 1H), 3.62 (s, 3H), 3.16-2.97 (m, 1H), 2.54-2.40 (m, 4H). MS (ESI, m/z): 194 (M+H)⁺

### Step E: Synthesis of (cis)-3-(hydroxymethyl)cyclobutane-1-sulfonamide

LiBH₄ (1.0 M in THF, 4.1 mL, 4.1 mmol) was added to a mixture of methyl (cis)-3-sulfamoylcyclobutane-1-carboxylate (200 mg, 1.04 mmol) in 2-propanol (2 mL) at 0° C. under an argon atmosphere. The mixture was stirred and heated for 1 hour at 40° C. The reaction was quenched with ethanol (0.5 mL) and concentrated in vacuo. The resulting residue was purified using silica gel chromatography (eluting methanol in dichloromethane) to provide (cis)-3-(hydroxymethyl)cyclobutane-1-sulfonamide (Int-24). ¹H NMR (300 MHz, CD₃CN) δ 3.78-3.63 (m, 1H), 3.44 (d, J=5.9 Hz, 2H), 2.46-2.33 (m, 1H), 2.31-2.21 (m, 2H), 2.13-2.02 (m, 2H).

### Synthesis of Intermediate Compound Int-25

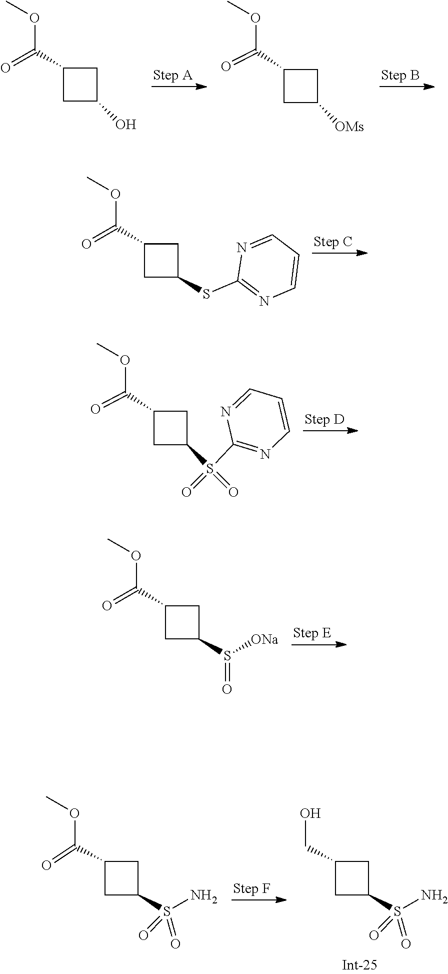

### Step A: Synthesis of methyl (cis)-3-((methylsulfonyl)oxy)cyclobutane-1-carboxylate

Methanesulfonyl chloride (5.39 mL, 69.2 mmol) was added to a mixture of methyl (cis)-3-hydroxycyclobutane-1-carboxylate (5.00 g, 38.4 mmol), and TEA (10.7 mL, 77.0 mmol) in DCM (60 mL) at 0° C. under an argon atmosphere. The mixture was allowed to stir for 2 hours at 25° C. The mixture was diluted with 1 M HCl (250 mL), and extracted with DCM (200 mL×3). The combined organic extracts were washed with brine (200 mL×3), dried over anhydrous sodium sulfate, filtered, and concentrated in vacuo to provide methyl (cis)-3-((methylsulfonyl)oxy)cyclobutane-1-carboxylate, which was used in the next step without purification.

### Step B: Synthesis of methyl (trans)-3-(pyrimidin-2-ylthio)cyclobutane-1-carboxylate

A mixture of methyl (cis)-3-((methylsulfonyl)oxy)cyclobutane-1-carboxylate (7.90 g, 37.9 mmol), pyrimidine-2-thiol (12.8 g, 114 mmol), and potassium carbonate (11.0 g, 80.0 mmol) in DMF (80 mL) was stirred and heated for 2 hours at 70° C. The mixture was diluted with water (500 mL), and extracted with ethyl acetate (500 mL×3). The combined organic extracts were washed with brine (200 mL×2), dried over anhydrous sodium sulfate, filtered, and concentrated in vacuo. The resulting residue was purified using silica gel chromatography (eluting ethyl acetate in petroleum ether) to provide methyl (trans)-3-(pyrimidin-2-ylthio)cyclobutane-1-carboxylate. ¹H NMR (300 MHz, CDCl₃) δ 8.48 (d, J=4.8 Hz, 2H), 6.94 (t, J=4.9 Hz, 1H), 4.43-4.23 (m, 1H), 3.71 (s, 3H), 3.43-3.17 (m, 1H), 2.96-2.78 (m, 2H), 2.47-2.25 (m, 2H). MS (ESI, m/z): 225 (M+H)⁺

### Step C: Synthesis of methyl (trans)-3-(pyrimidin-2-ylsulfonyl)cyclobutane-1-carboxylate

A mixture of mCPBA (6.09 g, 35.3 mmol), and methyl (trans)-3-(pyrimidin-2-ylthio)cyclobutane-1-carboxylate (3.60 g, 16.1 mmol) in DCM (120 mL) was allowed to stir for 16 hours at 25° C. The reaction was quenched with water (500 mL), and extracted with dichloromethane (500 mL×3). The combined organic extracts were washed with brine (300 mL×3), dried over anhydrous sodium sulfate, filtered, and concentrated in vacuo. The resulting residue was purified using reverse phase HPLC (eluting acetonitrile in water) to provide methyl (trans)-3-(pyrimidin-2-ylsulfonyl)cyclobutane-1-carboxylate. ¹H NMR (400 MHz, CDCl₃) δ 8.93 (d, J=4.9 Hz, 2H), 7.56 (t, J=4.8 Hz, 1H), 4.60-4.40 (m, 1H), 3.72 (s, 3H), 3.46-3.21 (m, 1H), 3.02-2.86 (m, 2H), 2.77-2.61 (m, 2H). MS (ESI, m/z): 257 (M+H)⁺

### Step D: Synthesis of sodium (trans)-3-(methoxycarbonyl)cyclobutane-1-sulfinate

Sodium methanolate (0.632 g, 11.7 mmol) was added to a mixture of methyl (trans)-3-(pyrimidin-2-ylsulfonyl)cyclobutane-1-carboxylate (3.00 g, 11.7 mmol) in MeOH (120 mL) at 25° C. under an argon atmosphere. The mixture was allowed to stir for 1 hour at 25° C. The mixture was concentrated in vacuo to provide sodium (trans)-3-(methoxycarbonyl)cyclobutane-1-sulfinate, which was used without purification in the next step.

### Step E: Synthesis of methyl (trans)-3-sulfamoylcyclobutane-1-carboxylate

Hydroxylamine-O-sulfonic acid (1.98 g, 17.5 mmol), and sodium acetate (1.92 g, 23.4 mmol) were added to a mixture of sodium (trans)-3-(methoxycarbonyl)cyclobutane-1-sulfinate (2.34 g, 11.7 mmol) in water (140 mL) at 25° C. under an argon atmosphere. The mixture was stirred and heated for 1 hour at 50° C. The mixture was purified using reverse phase HPLC (eluting acetonitrile in water) to provide give methyl (trans)-3-sulfamoylcyclobutane-1-carboxylate. ¹H NMR (300 MHz, CD₃CN) δ 3.85-3.72 (m, 1H), 3.66 (s, 3H), 3.32-3.11 (m, 1H), 2.65-2.48 (m, 4H). MS (ESI, m/z): 194 (M+H)⁺

### Step F: Synthesis of (trans)-3-(hydroxymethyl)cyclobutane-1-sulfonamide

LiBH₄ (2.0 M in THF, 5.2 mL, 10 mmol) was added to a mixture of methyl (trans)-3-sulfamoylcyclobutane-1-carboxylate (500 mg, 2.59 mmol) in 2-propanol (5 mL) at 0° C. under an argon atmosphere. The mixture was stirred and heated for 1 hour at 40° C. The reaction was quenched with ethanol (0.5 mL), and concentrated in vacuo. The resulting residue was purified using silica gel chromatography (eluting methanol in dichloromethane) to provide (trans)-3-(hydroxymethyl)cyclobutane-1-sulfonamide (Int-25). ¹H NMR (400 MHz, CD₃CN) δ 3.84-3.66 (m, 1H), 3.52 (d, J=6.4, 2.7 Hz, 2H), 2.56-2.43 (m, 1H), 2.42-2.30 (m, 2H), 2.24-2.06 (m, 2H).

### Synthesis of Intermediate Compound Int-26

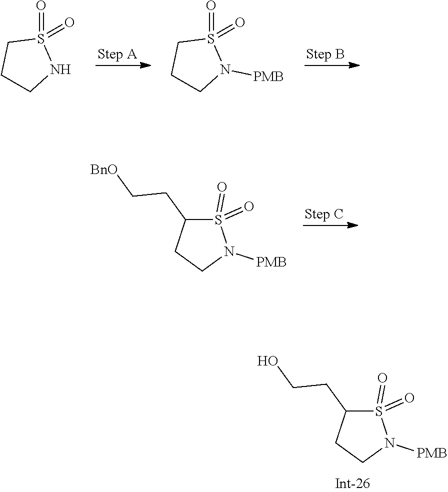

### Step A: Synthesis of 2-(4-methoxybenzyl)isothiazolidine 1,1-dioxide

A mixture of isothiazolidine 1,1-dioxide (3.00 g, 24.8 mmol), potassium carbonate (6.84 g, 49.5 mmol), and 1-(chloromethyl)-4-methoxybenzene (3.69 mL, 27.2 mmol) in acetonitrile (30 mL) was stirred and heated at 80° C. for 12 hours. The reaction was quenched with water (300 mL), and extracted with ethyl acetate (500 mL×3). The combined organic extracts were washed with brine (300 mL), dried over anhydrous sodium sulfate, filtered, and concentrated in vacuo. The resulting residue was purified using silica gel chromatography (eluting ethyl acetate in petroleum ether) to provide 2-(4-methoxybenzyl)isothiazolidine 1,1-dioxide. ¹H NMR (300 MHz, CDCl₃) δ 7.30-7.21 (m, 2H), 6.90-6.84 (m, 2H), 4.11 (s, 2H), 3.80 (s, 3H), 3.18 (t, J=7.8 Hz, 2H), 3.08 (t, J=6.8 Hz, 2H), 2.35-2.19 (m, 2H). MS (ESI, m/z): 242 (M+H)⁺

### Step B: Synthesis of 5-(2-(benzyloxy)ethyl)-2-(4-methoxybenzyl)isothiazolidine 1,1-dioxide

n-Butyllithium (2.5 M in THF, 5.0 mL, 13 mmol) was added to a mixture of 2-(4-methoxybenzyl)isothiazolidine 1,1-dioxide (2.00 g, 8.29 mmol) in THF (30 mL) at −78° C. under an argon atmosphere. The mixture was allowed to stir for 1 hour at −78° C. A solution of ((2-bromoethoxy)methyl)benzene (2.67 g, 12.4 mmol) in THF (6 mL) was added to the mixture at −78° C. The mixture was allowed to stir for 1 hour at −78° C. The reaction was quenched with saturated aqueous NH₄Cl (150 mL), and extracted with ethyl acetate (150 mL×3). The combined organic extracts were dried over anhydrous sodium sulfate, filtered, and concentrated in vacuo. The resulting residue was purified using silica gel chromatography (eluting ethyl acetate in petroleum ether) to provide 5-(2-(benzyloxy)ethyl)-2-(4-methoxybenzyl)isothiazolidine 1,1-dioxide. ¹H NMR (300 MHz, CDCl₃) δ 7.33 (s, 2H), 7.27-7.25 (m, 5H), 6.87 (d, J=8.5 Hz, 2H), 4.60-4.44 (m, 2H), 4.24-4.06 (m, 2H), 3.80 (s, 3H), 3.76-3.34 (m, 2H), 3.04-2.92 (m, 1H), 2.39-1.82 (m, 2H), 1.55 (s, 4H). MS (ESI, m/z): 376 (M+H)⁺

### Step C: Synthesis of 5-(2-hydroxyethyl)-2-(4-methoxybenzyl)isothiazolidine 1,1-dioxide

A mixture of 5-(2-(benzyloxy)ethyl)-2-(4-methoxybenzyl)isothiazolidine 1,1-dioxide (550 mg, 1.5 mmol), and Pd/C (550 mg, 0.52 mmol) in MeOH (15 mL) was stirred under a hydrogen atmosphere for 16 hours at 25° C. The mixture was filtered through Celite (washing with MeOH (100 mL×3)). The filtrate was concentrated in vacuo afford 5-(2-hydroxyethyl)-2-(4-methoxybenzyl)isothiazolidine 1,1-dioxide (Int-26), which was used without purification in the next step. ¹H NMR (300 MHz, CDCl₃) δ 7.28 (s, 1H), 7.25 (s, 1H), 6.90-6.85 (m, 2H), 4.23-4.08 (m, 2H), 3.98-3.75 (m, 5H), 3.49-3.36 (m, 1H), 3.09-2.96 (m, 2H), 2.47-2.17 (m, 2H), 2.07-1.84 (m, 2H). MS (ESI, m/z): 286 (M+H)⁺

### Synthesis of Intermediate Compound Int-27

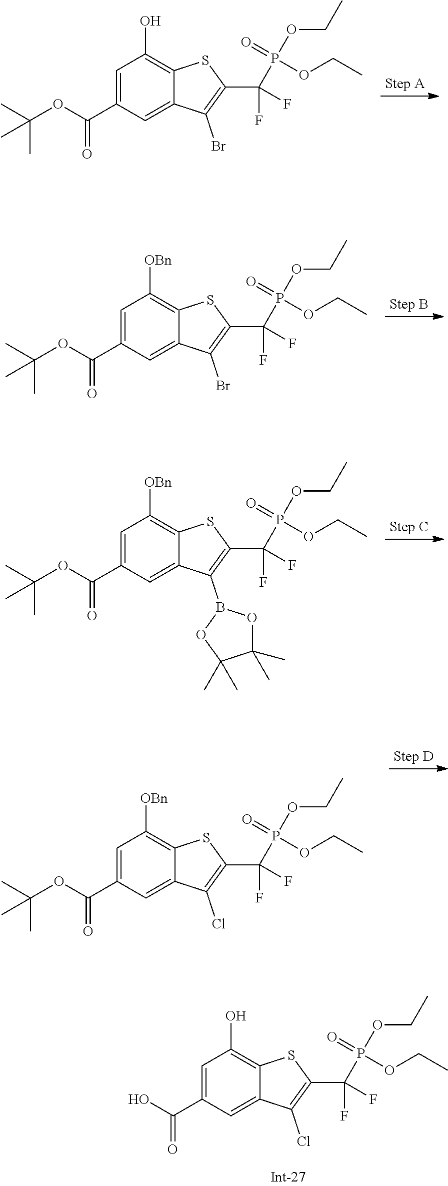

### Step A: Synthesis of tert-butyl 7-(benzyloxy)-3-bromo-2-((diethoxyphosphoryl)difluoromethyl) benzo[b]thiophene-5-carboxylate

A mixture of tert-butyl 3-bromo-2-((diethoxyphosphoryl)difluoromethyl)-7-hydroxybenzo[b]thiophene-5-carboxylate (500 mg, 0.970 mmol), potassium carbonate (201 mg, 1.46 mmol), and (bromomethyl)benzene (332 mg, 1.94 mmol) in DMF (5 mL) was allowed to stir for 16 hours at 25° C. The mixture was directly purified using reverse phase HPLC (eluting acetonitrile in water) to provide tert-butyl 7-(benzyloxy)-3-bromo-2-((diethoxyphosphoryl)difluoromethyl)benzo[b]thiophene-5-carboxylate. ¹H NMR (300 MHz, CDCl₃) δ 8.18 (s, 1H), 7.59 (s, 1H), 7.51-7.45 (m, 2H), 7.45-7.33 (m, 3H), 5.31 (s, 2H), 4.39-4.15 (m, 4H), 1.64 (s, 9H), 1.44-1.28 (m, 6H). MS (ESI, m/z): 605, 607 (M+H)⁺

### Step B: Synthesis of tert-butyl 7-(benzyloxy)-2-((diethoxyphosphoryl)difluoromethyl)-3-(4,4,5,5-tetramethyl-1,3,2-dioxaborolan-2-yl)benzo[b]thiophene-5-carboxylate

A mixture of tert-butyl 7-(benzyloxy)-3-bromo-2-((diethoxyphosphoryl)difluoromethyl) benzo[b]thiophene-5-carboxylate (400 mg, 0.661 mmol), 4,4,4′,4′,5,5,5′,5′-octamethyl-2,2′-bi(1,3,2-dioxaborolane) (419 mg, 1.65 mmol), potassium acetate (130 mg, 1.32 mmol), and dichlorobis(triphenylphosphine)palladium(II) (46 mg, 0.066 mmol) in toluene (4 mL) was stirred and heated at 100° C. for 18 hours. The mixture was concentrated in vacuo. The resulting residue was purified using reverse phase HPLC (eluting acetonitrile in water) to provide tert-butyl 7-(benzyloxy)-2-((diethoxyphosphoryl)difluoromethyl)-3-(4,4,5,5-tetramethyl-1,3,2-dioxaborolan-2-yl)benzo[b]thiophene-5-carboxylate. MS (ESI, m/z): 653 (M+H)⁺

### Step C: Synthesis of tert-butyl 7-(benzyloxy)-3-chloro-2-((diethoxyphosphoryl)difluoromethyl) benzo[b]thiophene-5-carboxylate

A mixture of tert-butyl 7-(benzyloxy)-2-((diethoxyphosphoryl)difluoromethyl)-3-(4,4,5,5-tetramethyl-1,3,2-dioxaborolan-2-yl)benzo[b]thiophene-5-carboxylate (200 mg, 0.307 mmol), and copper(II) chloride (82 mg, 0.61 mmol) in MeOH/water/MeCN (v:v=1:1:1;6 mL) was stirred and heated at 65° C. for 16 hours. The reaction was quenched with water (50 mL), and extracted with ethyl acetate (100 mL×2). The combined organic extracts were dried over anhydrous sodium sulfate, filtered, and concentrated in vacuo. The resulting residue was purified using silica gel chromatography (eluting ethyl acetate in petroleum ether) to provide tert-butyl 7-(benzyloxy)-3-chloro-2-((diethoxyphosphoryl)difluoromethyl)benzo[b]thiophene-5-carboxylate. ¹H NMR (300 MHz, CDCl₃) δ 8.16 (s, 1H), 7.59 (s, 1H), 7.52-7.31 (m, 5H), 5.31 (s, 2H), 4.39-4.21 (m, 4H), 1.64 (s, 9H), 1.43-1.30 (m, 6H). MS (ESI, m/z): 561 (M+H)⁺

### Step D: Synthesis of 3-chloro-2-((diethoxyphosphoryl)difluoromethyl)-7-hydroxybenzo[b]thiophene-5-carboxylic acid

Trichloroborane (1.0 M in DCM, 0.36 mL, 0.36 mmol) was added to a mixture of tert-butyl 7-(benzyloxy)-3-chloro-2-((diethoxyphosphoryl)difluoromethyl)benzo[b]thiophene-5-carboxylate (100 mg, 0.178 mmol) in DCM (1 mL) at −78° C. under an argon atmosphere. The mixture was allowed to stir for 2 hours at −78° C. The reaction was quenched with ethanol (2 mL). The mixture was concentrated in vacuo and The resulting residue was purified reverse phase HPLC (eluting acetonitrile in water) to provide 3-chloro-2-((diethoxyphosphoryl)difluoromethyl)-7-hydroxybenzo[b]thiophene-5-carboxylic acid. ¹H NMR (400 MHz, CD₃CN) δ 8.08 (s, 1H), 7.56 (s, 1H), 4.35-4.16 (m, 4H), 1.32 (t, J=7.0 Hz, 6H). MS (ESI, m/z): 415 (M+H)⁺

### Synthesis of Intermediate Compound Int-28

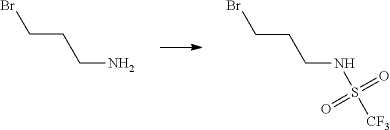 [SMILES: NCCCBr ; O=S(=O)(NCCCBr)C(F)(F)F]

Trifluoromethanesulfonic anhydride (1.29 g, 4.57 mmol) was added to a mixture of 3-bromopropan-1-amine hydrobromide (1.00 g, 4.57 mmol), and TEA (1.27 mL, 9.14 mmol) in DCM (10 mL) at 0° C. The mixture was allowed to stir for 2 hours at 25° C. The mixture was diluted with water (100 mL), and extracted with ethyl acetate (200 mL×3). The combined organic extracts were washed with brine (200 mL×2), dried over anhydrous sodium sulfate, filtered, and concentrated in vacuo. The resulting residue was purified using silica gel chromatography (eluting methanol in dichloromethane) to provide N-(3-bromopropyl)-1,1,1-trifluoromethanesulfonamide. ¹H NMR (400 MHz, DMSO-d₆) δ 9.49 (s, 1H), 3.56 (t, J=6.5 Hz, 2H), 3.28 (t, J=7.0 Hz, 2H), 2.08-1.94 (m, 2H).

### Synthesis of Intermediate Compound Int-29

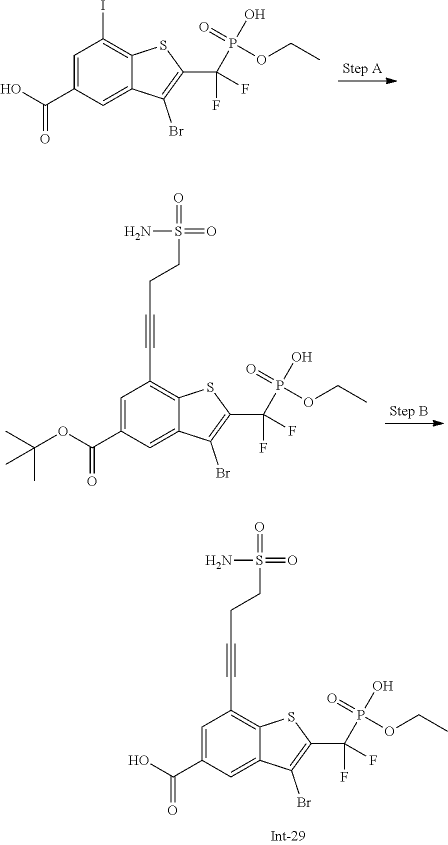

### Step A: Synthesis of tert-butyl 3-bromo-2-((ethoxy(hydroxy)phosphoryl)difluoromethyl)-7-(4-sulfamoylbut-1-yn-1-yl)benzo[b]thiophene-5-carboxylate

A mixture of tert-butyl 3-bromo-2-((diethoxyphosphoryl)difluoromethyl)-7-iodobenzo[b]thiophene-5-carboxylate (150 mg, 0.24 mmol), copper(I) iodide (5 mg, 0.02 mmol), TEA (0.100 mL, 0.720 mmol), dichlorobis(triphenylphosphine)palladium(II) (17 mg, 0.024 mmol), and but-3-yne-1-sulfonamide (48 mg, 0.36 mmol) in DMF (2 mL) was stirred at 25° C. for 16 hours. The mixture was purified using reverse phase HPLC (eluting acetonitrile in water) to provide tert-butyl 3-bromo-2-((ethoxy(hydroxy)phosphoryl)difluoromethyl)-7-(4-sulfamoylbut-1-yn-1-yl)benzo[b]thiophene-5-carboxylate. ¹H NMR (300 MHz, DMSO-d₆) δ 8.29 (s, 1H), 8.00 (s, 1H), 7.00 (s, 2H), 3.89-3.81 (m, 2H), 3.34-3.31 (m, 2H), 3.11-3.00 (m, 2H), 1.59 (s, 9H), 1.15 (t, J=7.2 Hz, 3H). MS (ESI, m/z): 602, 604 (M+H)⁺

### Step B: Synthesis of 3-bromo-2-((ethoxy(hydroxy)phosphoryl)difluoromethyl)-7-(4-sulfamoylbut-1-yn-1-yl)benzo[b]thiophene-5-carboxylic acid

A mixture of tert-butyl 3-bromo-2-((ethoxy(hydroxy)phosphoryl)difluoromethyl)-7-(4-sulfamoylbut-1-yn-1-yl)benzo[b]thiophene-5-carboxylate (10 mg, 0.02 mmol) in TFA (0.3 mL), and DCM (0.3 mL) was stirred at 25° C. for 2 hours. The mixture was concentrated in vacuo to provide 3-bromo-2-((ethoxy(hydroxy)phosphoryl)difluoromethyl)-7-(4-sulfamoylbut-1-yn-1-yl)benzo[b]thiophene-5-carboxylic acid (Int-29), which was used without purification in the next step. ¹H NMR (300 MHz, DMSO-d₆) δ 8.35 (s, 1H), 8.06 (s, 1H), 7.00 (s, 2H), 3.93-3.83 (m, 2H), 3.41-3.30 (m, 2H), 3.03 (t, J=7.4 Hz, 2H), 1.13 (t, J=7.0 Hz, 3H). MS (ESI, m/z): 546, 548 (M+H)⁺

### Synthesis of Intermediate Compound Int-30

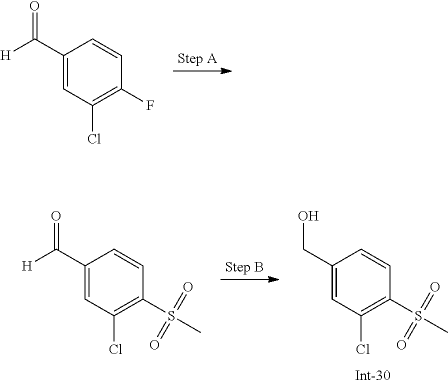

### Step A: Synthesis of 3-chloro-4-(methylsulfonyl)benzaldehyde

Sodium methanesulfinate (0.966 g, 9.46 mmol) was added to a mixture of 3-chloro-4-fluorobenzaldehyde (1.00 g, 6.31 mmol) in DMSO (10 mL) at room temperature under an argon atmosphere. The mixture was allowed to stir for 1 hr at 100° C. The reaction was quenched by the addition of water (300 mL), and extracted with ethyl acetate (500 mL×3). The combined organic extracts were washed with brine (300 mL×3), dried over anhydrous sodium sulfate, and filtered. The filtrate was concentrated in vacuo to provide 3-chloro-4-(methylsulfonyl)benzaldehyde, which was used without purification in the next step. MS (ESI, m/z): 217 (M−H)⁻

### Step B: Synthesis of (3-chloro-4-(methylsulfonyl)phenyl)methanol

NaBH₄ (130 mg, 3.43 mmol) was added to a mixture of 3-chloro-4-(methylsulfonyl)benzaldehyde (500 mg, 2.29 mmol) in EtOH (5 mL) at 0° C. under an argon atmosphere. The mixture was allowed to stir for 1 hour at room temperature. The reaction was quenched by the addition of saturated aqueous NH₄Cl (100 mL). The mixture was extracted with ethyl acetate (200 mL×3). The combined organic extracts were washed with brine (100 mL×3), dried over anhydrous sodium sulfate, and filtered. The filtrate was concentrated in vacuo and The resulting residue was purified using silica gel column chromatography (eluting methanol in dichloromethane) to provide (3-chloro-4-(methylsulfonyl)phenyl)methanol (Int-30). MS (ESI, m/z): 219 (M−H)⁻

### Synthesis of Intermediate Compound Int-31

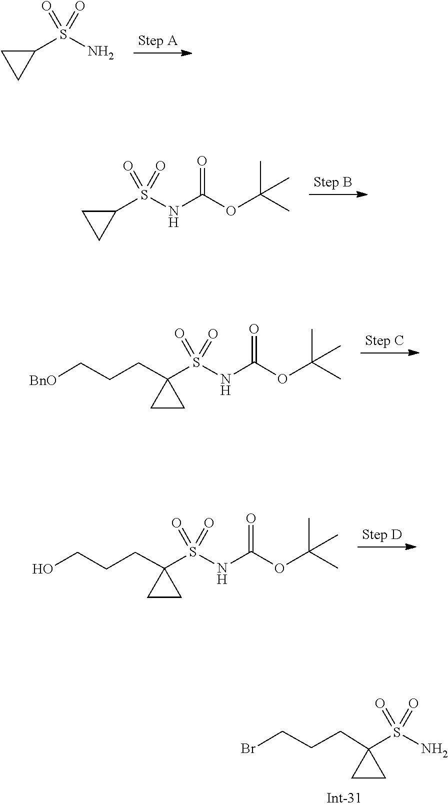 [SMILES: NS(=O)(=O)C1CC1 ; CC(C)(C)OC(=O)NS(=O)(=O)C1CC1 ; CC(C)(C)OC(=O)NS(=O)(=O)C1(CCCOCc2ccccc2)CC1 ; CC(C)(C)OC(=O)NS(=O)(=O)C1(CCCO)CC1 ; NS(=O)(=O)C1(CCCBr)CC1]

### Step A: Synthesis of tert-butyl (cyclopropylsulfonyl)carbamate

To a stirred solution of cyclopropanesulfonamide (2.00 g, 16.51 mmol), triethylamine (3.45 mL, 24.8 mmol), and 4-(dimethylamino)pyridine (0.202 g, 1.65 mmol) in dichloromethane (40 mL) was added di-tert-butyl dicarbonate (4.60 mL, 19.8 mmol) at room temperature. The mixture was allowed to stir for 3 hours at room temperature. The reaction was quenched by the addition of water (200 mL), and extracted with ethyl acetate (300 mL×3). The combined organic extracts were dried over anhydrous sodium sulfate, filtered, and concentrated in vacuo. The resulting residue was purified using silica gel column chromatography (eluting with ethyl acetate in petroleum ether) to provide tert-butyl (cyclopropylsulfonyl)carbamate. ¹H NMR (300 MHz, DMSO-d₆) δ 11.11 (s, 1H), 3.00-2.83 (m, 1H), 1.44 (s, 9H), 1.10-0.98 (m, 4H). MS (ESI, m/z): 220 (M−H)⁻

### Step B: Synthesis of tert-butyl ((1-(3-(benzyloxy)propyl)cyclopropyl)sulfonyl)carbamate

To a stirred solution of tert-butyl (cyclopropylsulfonyl)carbamate (500 mg, 2.26 mmol) in tetrahydrofuran (12.5 mL) was added n-butyllithium (2.5 M in THF, 2.71 mL, 6.8 mmol) at −78° C. under an argon atmosphere. The mixture was warmed to room temperature and stirred for 1.5 hours. ((3-Iodopropoxy)methyl)benzene (1.25 g, 4.52 mmol) was added to the mixture at −78° C. The mixture was warmed to room temperature and stirred for 16 hours. The reaction was quenched by the addition of water (100 mL), and extracted with ethyl acetate (200 mL×3). The organic fractions were combined, dried over anhydrous sodium sulfate, filtered, and concentrated in vacuo. The resulting residue was purified using silica gel column chromatography (eluting with ethyl acetate in petroleum ether) to provide tert-butyl ((1-(3-(benzyloxy)propyl)cyclopropyl)sulfonyl)carbamate. ¹H NMR (400 MHz, DMSO-d₆) δ 11.04 (s, 1H), 7.39-7.24 (m, 5H), 4.44 (s, 2H), 3.42 (t, J=6.1 Hz, 2H), 1.86-1.79 (m, 2H), 1.76-1.66 (m, 2H), 1.42 (s, 9H), 1.35-1.29 (m, 2H), 1.00-0.91 (m, 2H). MS (ESI, m/z): 368 (M−H)⁻

### Step C: Synthesis of tert-butyl ((1-(3-hydroxypropyl)cyclopropyl)sulfonyl)carbamate

To a solution of tert-butyl ((1-(3-(benzyloxy)propyl)cyclopropyl)sulfonyl)carbamate (230 mg, 0.623 mmol) in tetrahydrofuran (10 mL) was added palladium on carbon (10%, 1150 mg) at room temperature. The mixture was allowed to stir for 1 hour under a hydrogen atmosphere. The mixture was filtered and the filtrate was directly purified using silica gel column chromatography (eluting with ethyl acetate in petroleum ether) to provide tert-butyl ((1-(3-hydroxypropyl)cyclopropyl)sulfonyl)carbamate. ¹H NMR (300 MHz, DMSO-d₆) δ 11.00 (s, 1H), 4.42 (t, J=5.0 Hz, 1H), 3.43-3.33 (m, 2H), 1.84-1.73 (m, 2H), 1.62-1.50 (m, 2H), 1.43 (s, 9H), 1.35-1.28 (m, 2H), 0.97-0.88 (m, 2H). MS (ESI, m/z): 278 (M−H)⁻

### Step D: Synthesis of 1-(3-bromopropyl)cyclopropane-1-sulfonamide

To a solution of tert-butyl ((1-(3-hydroxypropyl)cyclopropyl)sulfonyl)carbamate (100 mg, 0.358 mmol) in DCM (0.5 mL) was added tribromophosphane (194 mg, 0.716 mmol) at 0° C. under an argon atmosphere. The mixture was heated to 35° C., and allowed to stir for 2 hours under a hydrogen atmosphere. The reaction was quenched by the addition of water (30 mL), and extracted with ethyl acetate (50 mL×3). The combined organic extracts were washed with saturated aqueous sodium bicarbonate (100 mL), dried over anhydrous sodium sulfate, filtered, and concentrated in vacuo. The resulting residue was purified using silica gel column chromatography (eluting with ethyl acetate in petroleum ether) to provide 1-(3-bromopropyl)cyclopropane-1-sulfonamide (Int-31). ¹H NMR (300 MHz, DMSO-d₆) δ 6.82 (s, 2H), 3.53 (t, J=6.6 Hz, 2H), 2.11-1.95 (m, 2H), 1.95-1.80 (m, 2H), 1.19-1.08 (m, 2H), 0.88-0.75 (m, 2H).

### Synthesis of Intermediate Compound Int-32

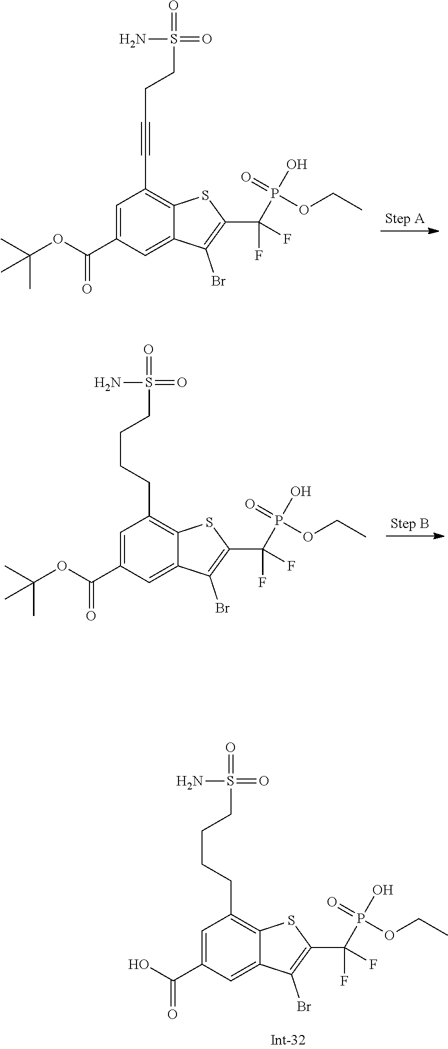

### Step A: Synthesis of tert-butyl 3-bromo-2-((ethoxy(hydroxy)phosphoryl)difluoromethyl)-7-(4-sulfamoylbutyl)benzo[b]thiophene-5-carboxylate

To a mixture of tert-butyl 3-bromo-2-((ethoxy(hydroxy)phosphoryl)difluoromethyl)-7-(4-sulfamoylbut-1-yn-1-yl)benzo[b]thiophene-5-carboxylate (160 mg, 0.266 mmol) in MeOH (5 mL) was added platinum (IV) oxide (80 mg, 0.35 mmol) at room temperature under an argon atmosphere. The resulting reaction was allowed to stir at room temperature for 1 hour under a hydrogen atmosphere. The mixture was filtered and concentrated in vacuo. The resulting residue was purified using reverse phase HPLC (eluting acetonitrile in water) to provide tert-butyl 3-bromo-2-((ethoxy(hydroxy)phosphoryl)difluoromethyl)-7-(4-sulfamoylbutyl)benzo[b]thiophene-5-carboxylate. MS (ESI, m/z): 604, 606 (M−H)⁻

### Step B: Synthesis of 3-bromo-2-((ethoxy(hydroxy)phosphoryl)difluoromethyl)-7-(4-sulfamoylbutyl)benzo[b]thiophene-5-carboxylic acid

A mixture of tert-butyl 3-bromo-2-((ethoxy(hydroxy)phosphoryl)difluoromethyl)-7-(4-sulfamoylbutyl)benzo[b]thiophene-5-carboxylate (85 mg, 0.14 mmol) in TFA (1.00 mL, 13.0 mmol), and DCM (1 mL) was stirred at room temperature for 3 hours. The mixture was concentrated in vacuo to provide 3-bromo-2-((ethoxy(hydroxy)phosphoryl)difluoromethyl)-7-(4-sulfamoylbutyl)benzo[b]thiophene-5-carboxylic acid (Int-32), which was used without purification in the next step. MS (ESI, m/z): 548, 550 (M−H)⁻

### Synthesis of Intermediate Compound Int-33

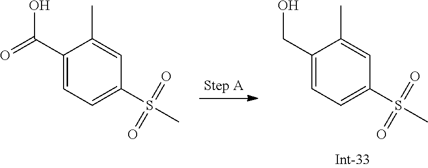

### Step A: Synthesis of (2-methyl-4-(methylsulfonyl)phenyl)methanol

A solution of borane (1.0 M in THF, 9.34 mL, 9.3 mmol) was added dropwise to a mixture of 2-methyl-4-(methylsulfonyl)benzoic acid (200 mg, 0.934 mmol) in THF (2 mL) at 0° C. under an argon atmosphere. The mixture was allowed to stir for 2 hours at room temperature. The reaction was quenched with methanol (10 mL) at 0° C. The mixture was diluted with water (50 mL), and extracted with ethyl acetate (100 mL×3). The combined organic extracts were washed with brine (50 mL×2), dried over anhydrous sodium sulfate, filtered, and concentrated in vacuo. The resulting residue was purified using silica gel column chromatography (eluting with ethyl acetate in petroleum ether) to provide (2-methyl-4-(methylsulfonyl)phenyl)methanol (Int-33). ¹H NMR (300 MHz, DMSO-d₆) δ 7.76-7.60 (m, 3H), 4.57 (d, J=5.4 Hz, 2H), 3.17 (s, 3H), 2.31 (s, 3H). MS (ESI, m/z): 199 (M−H)⁻

### Synthesis of Intermediate Compound Int-34

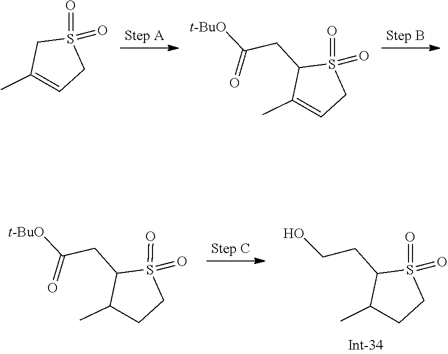

### Step A: Synthesis of tert-butyl 2-(3-methyl-1,1-dioxido-2,5-dihydrothiophen-2-yl)acetate

To a mixture of 3-methyl-2,5-dihydrothiophene 1,1-dioxide (2.00 g, 15.1 mmol) in THF (4 mL) was added sodium bis(trimethylsilyl)amide (2.0 M in THF, 4.0 mL, 8.0 mmol) at −78° C. under an argon atmosphere. The mixture was allowed to stir for 1 hour at −78° C. To the mixture was added tert-butyl 2-bromoacetate (8.85 g, 45.4 mmol) at −78° C. under an argon atmosphere. The mixture was allowed to stir for 1 hour at −78° C. The reaction was quenched with saturated aqueous ammonium chloride (100 mL), and extracted with dichloromethane (100 mL×3). The combined organic extracts were washed with brine (100 mL×3), dried over anhydrous sodium sulfate, filtered, and concentrated in vacuo. The resulting residue was purified using silica gel column chromatography (eluting ethyl acetate in petroleum ether) to provide tert-butyl 2-(3-methyl-1,1-dioxido-2,5-dihydrothiophen-2-yl)acetate. ¹H NMR (300 MHz, CDCl₃) δ 5.75-5.59 (m, 1H), 4.00 (t, J=7.2 Hz, 1H), 3.77-3.68 (m, 2H), 2.85-2.52 (m, 2H), 1.84 (s, 3H), 1.49 (s, 9H).

### Step B: Synthesis of tert-butyl 2-(3-methyl-1,1-dioxidotetrahydrothiophen-2-yl)acetate

A mixture of tert-butyl 2-(3-methyl-1,1-dioxido-2,5-dihydrothiophen-2-yl)acetate (440 mg, 1.79 mmol), and Pd/C (10%, 65 mg) in methanol (21 mL) was stirred under a hydrogen atmosphere for 1 hour at room temperature. The mixture was filtered and the filtrate was concentrated in vacuo to provide tert-butyl 2-(3-methyl-1,1-dioxidotetrahydrothiophen-2-yl)acetate, which was used without purification in the next step. ¹H NMR (300 MHz, CDCl₃) δ 3.60-2.70 (m, 4H), 2.55-1.55

### Step C: Synthesis of 2-(2-hydroxyethyl)-3-methyltetrahydrothiophene 1,1-dioxide

Lithium aluminum hydride (2.4 M in THF, 0.93 mL, 2.2 mmol) was added to a mixture of tert-butyl 2-(3-methyl-1,1-dioxidotetrahydrothiophen-2-yl)acetate (371 mg, 1.494 mmol) in THF (7.4 mL) at 0° C. under an argon atmosphere. The resulting mixture was allowed to stir for 2 hours at 0° C. The reaction was quenched with water (50 mL), and extracted with ethyl acetate (50 mL×3). The organic layers were washed with brine (50 mL×3), dried over anhydrous sodium sulfate, filtered, and concentrated in vacuo. The resulting residue was purified using silica gel column chromatography (eluting ethyl acetate in petroleum ether) to provide 2-(2-hydroxyethyl)-3-methyltetrahydrothiophene 1,1-dioxide (Int-34). ¹H NMR (300 MHz, CDCl₃) δ 3.97-3.76 (m, 2H), 3.33-2.92 (m, 2H), 2.84-2.55 (m, 1H), 2.38-2.15 (m, 1H), 2.07-1.95 (m, 2H), 1.83-1.75 (m, 2H), 1.19-1.11 (m, 3H).

### Synthesis of Intermediate Compound Int-35

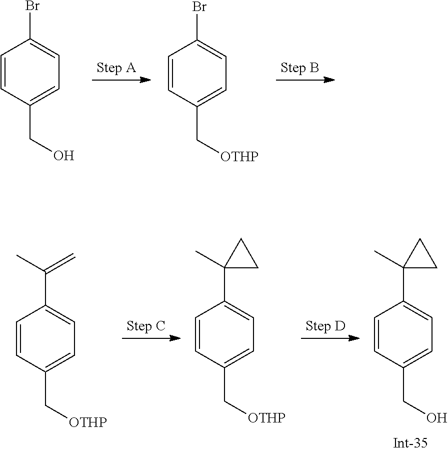

### Step A: Synthesis of 2-((4-bromobenzyl)oxy)tetrahydro-2H-pyran

A mixture of (4-bromophenyl)methanol (1.00 g, 5.35 mmol), pyridinium p-toluenesulfonate (0.269 g, 1.07 mmol), and 3,4-dihydro-2H-pyran (0.587 mL, 6.42 mmol) in DCM (10 mL) was stirred at room temperature for 16 hours. The reaction was quenched with water (300 mL), and extracted with ethyl acetate (500 mL×3). The combined organic extracts were washed with brine (300 mL×2), dried over anhydrous sodium sulfate, filtered, and concentrated in vacuo. The resulting residue was purified using silica gel chromatography (eluting ethyl acetate in petroleum ether) to provide 2-((4-bromobenzyl)oxy)tetrahydro-2H-pyran. ¹H NMR (300 MHz, CDCl₃) δ 7.47 (d, J=8.3 Hz, 2H), 7.24 (d, J=8.2 Hz, 2H), 4.78-4.64 (m, 2H), 4.45 (d, J=12.3 Hz, 1H), 3.96-3.83 (m, 1H), 3.60-3.49 (m, 1H), 1.94-1.97-1.46 (m, 6H).

### Step B: Synthesis of 2-((4-(prop-1-en-2-yl)benzyl)oxy)tetrahydro-2H-pyran

A mixture of 2-((4-bromobenzyl)oxy)tetrahydro-2H-pyran (2.4 g, 8.9 mmol), [1,1′-bis(diphenylphosphino)ferrocene]dichloropalladium(II) (0.971 g, 1.33 mmol), 4,4,5,5-tetramethyl-2-(prop-1-en-2-yl)-1,3,2-dioxaborolane (2.23 g, 13.3 mmol), and potassium phosphate tribasic (5.64 g, 26.6 mmol) in 1,4-dioxane (20 mL), and water (4 mL) was stirred at 100° C. for 16 hours. The reaction was quenched with water (300 mL), and extracted with ethyl acetate (300 mL×3). The combined organic extracts were washed with brine (300 mL), dried over anhydrous sodium sulfate, filtered, and concentrated in vacuo. The resulting residue was purified using silica gel chromatography (eluting ethyl acetate in petroleum ether) to provide 2-((4-(prop-1-en-2-yl)benzyl)oxy)tetrahydro-2H-pyran. ¹H NMR (300 MHz, CDCl₃) δ 7.50-7.40 (m, 2H), 7.33 (d, J=8.3 Hz, 2H), 5.40-5.33 (m, 1H), 5.07 (s, 1H), 4.84-4.69 (m, 2H), 4.50 (d, J=12.0 Hz, 1H), 4.00-3.86 (m, 1H), 3.55 (d, J=10.1 Hz, 1H), 2.15 (s, 3H), 1.95-1.48 (m, 6H).

### Step C: Synthesis of 2-((4-(1-methylcyclopropyl)benzyl)oxy)tetrahydro-2H-pyran

A solution of trifluoroacetic acid (0.464 mL, 6.03 mmol) in DCM (6 mL) was added to a mixture of diethylzinc (1.49 g, 12.1 mmol) in DCM (1 mL) at 0° C. The mixture was allowed to stir for 30 min at 0° C. A solution of diiodomethane (1.61 g, 6.03 mmol) in DCM (6 mL) was then added at 0° C. The mixture was allowed to stir for 20 min at 0° C. A solution of 2-((4-(prop-1-en-2-yl)benzyl)oxy)tetrahydro-2H-pyran (700 mg, 3.01 mmol) in DCM (6 mL) was added to the mixture at 0° C. The mixture was allowed to stir for 16 hours at 0° C. The reaction was quenched with brine (60 mL), and extracted with ethyl acetate (200 mL×3). The combined organic extracts were washed with brine (200 mL), dried over anhydrous sodium sulfate, filtered, and concentrated in vacuo. The resulting residue was purified using silica gel chromatography (eluting ethyl acetate in petroleum ether) to provide 2-((4-(1-methylcyclopropyl)benzyl)oxy)tetrahydro-2H-pyran. ¹H NMR (300 MHz, CDCl₃) δ 7.40-7.18 (m, 4H), 4.85-4.62 (m, 2H), 4.56-4.38 (m, 1H), 3.99-3.86 (m, 1H), 3.59-3.48 (m, 1H), 1.80-1.49 (m, 6H), 1.40 (s, 3H), 0.90-0.63 (m, 4H).

### Step D: Synthesis of (4-(1-methylcyclopropyl)phenyl)methanol

A mixture of 2-((4-(1-methylcyclopropyl)benzyl)oxy)tetrahydro-2H-pyran (270 mg, 1.10 mmol) in hydrochloric acid (2.0 M in ethyl acetate, 5.0 mL, 10 mmol) was stirred at room temperature for 3 hours. The mixture was diluted with water (100 mL), and extracted with ethyl acetate (100 mL×3). The combined organic extracts were washed with brine (100 mL), dried over anhydrous sodium sulfate, filtered, and concentrated in vacuo. The resulting residue was purified using silica gel chromatography (eluting ethyl acetate in petroleum ether) to provide (4-(1-methylcyclopropyl)phenyl)methanol (Int-35). ¹H NMR (300 MHz, CDCl₃) δ 7.33-7.20 (m, 4H), 4.65 (s, 2H), 1.40 (s, 3H), 0.87-0.81 (m, 2H), 0.77-0.68 (m, 2H).

### Synthesis of Intermediate Compound Int-36

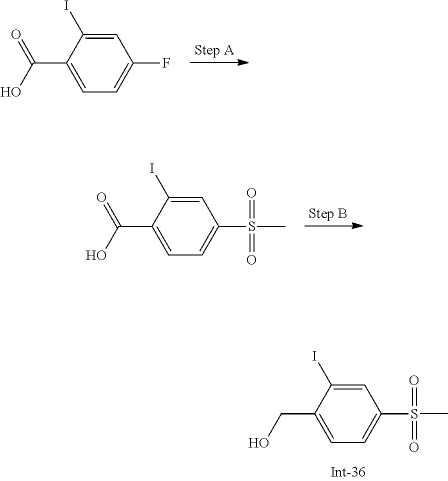

### Step A: Synthesis of 2-iodo-4-(methylsulfonyl)benzoic acid

Sodium methanesulfinate (1.54 g, 15.0 mmol) was added to a mixture of 4-fluoro-2-iodobenzoic acid (2.00 g, 7.52 mmol) in DMSO (20 mL) at room temperature under an argon atmosphere. The mixture was allowed to stir for 16 hours at 100° C. The mixture was diluted with water (500 mL). The pH of the mixture was adjusted to 2-3 by the addition of aqueous hydrochloric acid (1.0 M, 10 mL, 10 mmol), and then extracted with ethyl acetate (800 mL×3). The combined organic extracts were dried over anhydrous sodium sulfate, filtered, and concentrated in vacuo to provide 2-iodo-4-(methylsulfonyl)benzoic acid, which was used without purification in the next step. ¹H NMR (400 MHz, DMSO-d₆) δ 13.86 (s, 1H), 8.41 (d, J=1.8 Hz, 1H), 8.01 (dd, J=8.0, 1.8 Hz, 1H), 7.87 (d, J=8.1 Hz, 1H), 3.31 (s, 3H). MS (ESI, m/z): 325 (M−H)⁻

### Step B: Synthesis of (2-iodo-4-(methylsulfonyl)phenyl)methanol

A solution of borane (1.0 M in THF, 15.3 mL, 15 mmol) was added to a mixture of 2-iodo-4-(methylsulfonyl)benzoic acid (500 mg, 1.53 mmol) in THF (5 mL) at 0° C. under an argon atmosphere. The mixture was warmed to room temperature and stirred for 1 hour. The reaction was quenched with methanol (50 mL), diluted with water (200 mL), and extracted with ethyl acetate (300 mL×3). The combined organic extracts were washed with brine (200 mL×2), dried with anhydrous sodium sulfate, filtered, and concentrated in vacuo. The resulting residue was purified using silica gel chromatography (eluting ethyl acetate in petroleum ether) to provide (2-iodo-4-(methylsulfonyl)phenyl)methanol (Int-36). ¹H NMR (400 MHz, CDCl₃) δ 8.33 (d, J=1.9 Hz, 1H), 7.92 (dd, J=8.1, 1.9 Hz, 1H), 7.71 (d, J=8.1 Hz, 1H), 4.72 (s, 2H), 3.06 (s, 3H). MS (ESI, m/z): 311 (M−H)⁻

### Synthesis of Intermediate Compound Int-37

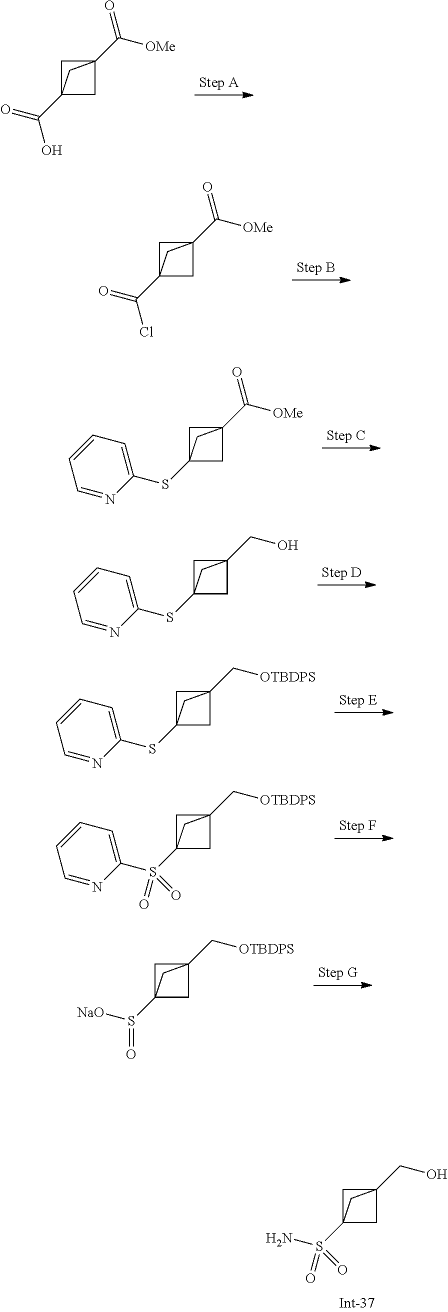

### Step A: Synthesis of methyl 3-(chlorocarbonyl)bicyclo[1.1.1]pentane-1-carboxylate

A mixture of 3-(methoxycarbonyl)bicyclo[1.1.1]pentane-1-carboxylic acid (5.00 g, 29.4 mmol) in thionyl chloride (30 mL, 411 mmol) was stirred at 85° C. for 2 hours. The mixture was concentrated in vacuo to provide methyl 3-(chlorocarbonyl)bicyclo[1.1.1]pentane-1-carboxylate, which was used in the next step without purification.

### Step B: Synthesis of methyl 3-(pyridin-2-ylthio)bicyclo[1.1.1]pentane-1-carboxylate

A mixture of methyl 3-(chlorocarbonyl)bicyclo[1.1.1]pentane-1-carboxylate (5.55 g, 29.4 mmol) in benzene (30.0 mL) was added to a mixture of sodium 2-thioxopyridin-1(2H)-olate (5.26 g, 35.3 mmol) in benzene (30 mL) at 82° C. The resulting reaction was heated to 82° C. for 2 hours. The mixture was concentrated in vacuo. The resulting residue was purified using silica gel chromatography (eluting ethyl acetate in petroleum ether) to provide methyl 3-(pyridin-2-ylthio)bicyclo[1.1.1]pentane-1-carboxylate. ¹H NMR (300 MHz, CDCl₃) δ 8.47 (s, 1H), 7.59-7.48 (m, 1H), 7.34-7.19 (m, 1H), 7.11-7.00 (m, 1H), 3.70 (s, 3H), 2.47 (s, 6H). MS (ESI, m/z): 236 (M+H)⁺

### Step C: Synthesis of (3-(84yridine-2-ylthio)bicyclo[1.1.1]pentan-1-yl)methanol

A mixture of methyl 3-(84yridine-2-ylthio)bicyclo[1.1.1]pentane-1-carboxylate (1.5 g, 6.4 mmol), and sodium borohydride (0.362 g, 9.56 mmol) in methanol (15 mL) was stirred at room temperature for 2 hours. The reaction was quenched with saturated aqueous ammonium chloride (250 mL), and extracted with ethyl acetate (250 mL×3). The combined organic extracts were washed with brine (250 mL), dried over anhydrous sodium sulfate, filtered, and concentrated in vacuo. The resulting residue was purified using silica gel chromatography (eluting ethyl acetate in petroleum ether) to provide (3-(84yridine-2-ylthio)bicyclo[1.1.1]pentan-1-yl)methanol. ¹H NMR (300 MHz, CDCl₃) δ 8.50-8.42 (m, 1H), 7.59-7.47 (m, 1H), 7.32-7.23 (m, 1H), 7.10-7.00 (m, 1H), 3.68 (s, 2H), 2.13 (s, 6H). MS (ESI, m/z): 208 (M+H)⁺

### Step D: Synthesis of 2-((3-(((tert-butyldiphenylsilyl)oxy)methyl)bicyclo[1.1.1]pentan-1-yl)thio)pyridine

To a mixture of (3-(84yridine-2-ylthio)bicyclo[1.1.1]pentan-1-yl)methanol (1.1 g, 5.3 mmol) in dichloromethane (20 mL) were added tert-butylchlorodiphenylsilane (1.60 g, 5.84 mmol), N,N-dimethylpyridin-4-amine (0.065 g, 0.53 mmol), and triethylamine (1.11 mL, 7.96 mmol) at 0° C. The mixture was warmed to room temperature and stirred for 2 hours. The reaction was quenched with water (200 mL), and extracted with ethyl acetate (200 mL×3). The combined organic layers were washed with brine (300 mL), dried over anhydrous sodium sulfate, filtered, and concentrated in vacuo. The resulting residue was purified using silica gel chromatography (eluting ethyl acetate in petroleum ether) to provide 2-((3-(((tert-butyldiphenylsilyl)oxy)methyl)bicyclo[1.1.1]pentan-1-yl)thio)pyridine. ¹H NMR (300 MHz, CDCl₃) δ 8.46 (s, 1H), 7.74-7.59 (m, 5H), 7.57-7.51 (m, 1H), 7.46-7.33 (m, 4H), 7.33-7.23 (m, 2H), 7.05 (s, 1H), 3.69 (s, 2H), 2.08 (s, 6H), 1.05 (s, 9H). MS (ESI, m/z): 446 (M+H)⁺

### Step E: Synthesis of 2-((3-(((tert-butyldiphenylsilyl)oxy)methyl)bicyclo[1.1.1]pentan-1-yl)sulfonyl)pyridine

To a mixture of 2-((3-(((tert-butyldiphenylsilyl)oxy)methyl)bicyclo[1.1.1]pentan-1-yl)thio)pyridine (2.2 g, 4.9 mmol) in dichloromethane (25 mL) was added 3-chloroperoxybenzoic acid (1.87 g, 10.9 mmol) at 0° C. The mixture was warmed to room temperature and stirred for 16 hours. The reaction was quenched with saturated aqueous sodium bicarbonate (100 mL), and extracted with ethyl acetate (300 mL×3). The combined organic layers were washed with brine (300 mL), dried over anhydrous sodium sulfate, filtered, and concentrated in vacuo. The resulting residue was purified using silica gel chromatography (eluting ethyl acetate in petroleum ether) to provide 2-((3-(((tert-butyldiphenylsilyl)oxy)methyl)bicyclo[1.1.1]pentan-1-yl)sulfonyl)pyridine. ¹H NMR (300 MHz, CDCl₃) δ 8.79 (s, 1H), 8.11-8.03 (m, 1H), 8.02-7.90 (m, 1H), 7.64-7.52 (m, 5H), 7.44-7.32 (m, 6H), 3.66 (s, 2H), 2.06 (s, 6H), 1.02 (s, 9H). MS (ESI, m/z): 478 (M−H)⁻

### Step F: Synthesis of sodium 3-(((tert-butyldiphenylsilyl)oxy)methyl)bicyclo[1.1.1]pentane-1-sulfinate

To a mixture of 2-((3-(((tert-butyldiphenylsilyl)oxy)methyl)bicyclo[1.1.1]pentan-1-yl)sulfonyl)pyridine (1.2 g, 2.5 mmol) in methanol (25 mL) was added a solution of sodium methylate (30% in methanol, 1.81 g, 10.1 mmol) at room temperature. The mixture was stirred and heated to 40° C. for 16 hours. The mixture was concentrated in vacuo to provide sodium 3-(((tert-butyldiphenylsilyl)oxy)methyl)bicyclo[1.1.1]pentane-1-sulfinate, which was used without purification in the next step. MS (ESI, m/z): 399 (M−H)⁻

### Step G: Synthesis of 3-(hydroxymethyl)bicyclo[1.1.1]pentane-1-sulfonamide

To a mixture of sodium 3-(((tert-butyldiphenylsilyl)oxy)methyl)bicyclo[1.1.1]pentane-1-sulfinate (1.06 g, 2.51 mmol) in water (50 mL) were added hydroxylamine-o-sulfonic acid (426 mg, 3.77 mmol), and sodium acetate (412 mg, 5.02 mmol) at room temperature. The mixture was stirred and heated to 50° C. for 16 hours. The mixture was diluted with water (150 mL), and extracted with ethyl acetate (200 mL×3). The combined organic layers were washed with brine (300 mL), dried over anhydrous sodium sulfate, filtered, and concentrated in vacuo. The resulting residue was purified using silica gel chromatography (eluting methanol in dichloromethane) to provide 3-(hydroxymethyl)bicyclo[1.1.1]pentane-1-sulfonamide (Int-37). ¹H NMR (300 MHz, DMSO-d₆) δ 6.75 (s, 2H), 4.65 (t, J=5.4 Hz, 1H), 3.48-3.41 (m, 2H), 1.88 (s, 6H). MS (ESI, m/z): 176 (M−H)⁻

### Synthesis of Intermediate Compound Int-38

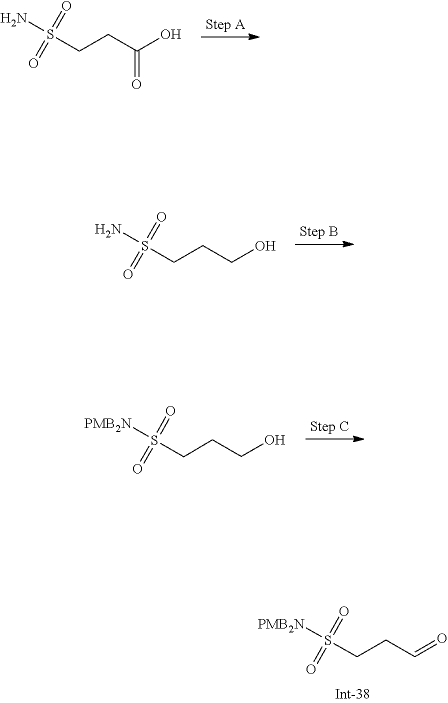

### Step A: Synthesis of 3-hydroxypropane-1-sulfonamide

A solution of borane (1.0 M in THF, 11.8 mL, 12 mmol) was added to a mixture of 3-sulfamoylpropanoic acid (200 mg, 1.31 mmol) in THF (5 mL) at 0° C. under an argon atmosphere. The mixture was warmed to room temperature and stirred for 1 hour. The reaction was quenched with methanol (5 mL), diluted with water (100 mL), and extracted with ethyl acetate (100 mL×3). The combined organic extracts were washed with brine (100 mL×2), dried over anhydrous sodium sulfate, filtered, and concentrated in vacuo. The resulting residue was purified using silica gel chromatography (eluting methanol in dichloromethane) to provide 3-hydroxypropane-1-sulfonamide. ¹H NMR (400 MHz, DMSO-d₆) δ 6.72 (s, 1H), 3.18-3.11 (m, 2H), 3.01-2.95 (m, 2H), 2.29-2.20 (m, 2H). MS (ESI, m/z): 138 (M−H)⁻

### Step B: Synthesis of 3-hydroxy-N,N-bis(4-methoxybenzyl)propane-1-sulfonamide

1-(Chloromethyl)-4-methoxybenzene (0.117 mL, 0.862 mmol) was added to a mixture of 3-hydroxypropane-1-sulfonamide (80 mg, 0.58 mmol), and cesium carbonate (562 mg, 1.73 mmol) in DMF (0.8 mL). The mixture was stirred and heated to 50° C. for 1 hour. The mixture was concentrated in vacuo and The resulting residue was purified using silica gel chromatography (eluting methanol in dichloromethane) to provide 3-hydroxy-N,N-bis(4-methoxybenzyl)propane-1-sulfonamide. ¹H NMR (300 MHz, DMSO-d₆) δ 6.81-6.75 (m, 4H), 6.53-6.46 (m, 4H), 3.81 (s, 4H), 3.35 (s, 6H), 3.10-3.00 (m, 2H), 2.73-2.64 (m, 2H), 1.47-1.36 (m, 2H). MS (ESI, m/z): 380 (M+H)⁺

### Step C: Synthesis of N,N-bis(4-methoxybenzyl)-3-oxopropane-1-sulfonamide

To a solution of 3-hydroxy-N,N-bis(4-methoxybenzyl)propane-1-sulfonamide (250 mg, 0.659 mmol) in DCM (4 mL) was added Dess-Martin periodinane (419 mg, 0.988 mmol). The mixture was allowed to stir for 2 hours at room temperature. The reaction was quenched with saturated aqueous sodium thiosulfate (30 mL), and extracted with ethyl acetate (40 mL×3). The combined organic layers were washed with brine (70 mL), dried over anhydrous sodium sulfate, filtered, and concentrated in vacuo. The resulting residue was purified using silica gel chromatography (eluting ethyl acetate in petroleum ether) to provide N,N-bis(4-methoxybenzyl)-3-oxopropane-1-sulfonamide (Int-38). ¹H NMR (400 MHz, CDCl₃) δ 9.73 (s, 1H), 7.23-7.19 (m, 4H), 6.90-6.86 (m, 4H), 4.27 (s, 4H), 3.81 (s, 6H), 3.16 (t, J=7.3 Hz, 2H), 2.95 (t, J=7.3 Hz, 2H). MS (ESI, m/z): 378 (M+H)⁺

### Synthesis of Intermediate Compound Int-39

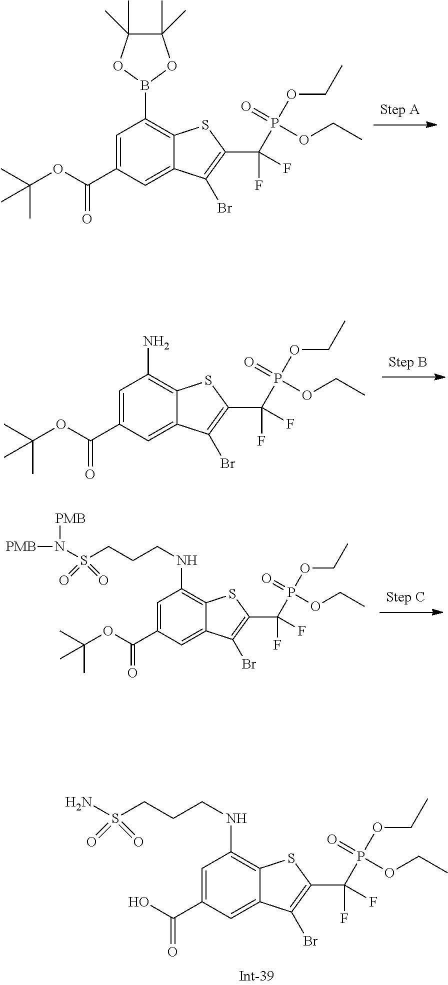

### Step A: Synthesis of tert-butyl 7-amino-3-bromo-2-((diethoxyphosphoryl)difluoromethyl) benzo[b]thiophene-5-carboxylate

A mixture of tert-butyl 3-bromo-2-((diethoxyphosphoryl)difluoromethyl)-7-(4,4,5,5-tetramethyl-1,3,2-dioxaborolan-2-yl)benzo[b]thiophene-5-carboxylate (400 mg, 0.640 mmol), 0-(2,4-dinitrophenyl)hydroxylamine (191 mg, 0.960 mmol), and cesium carbonate (313 mg, 0.960 mmol) in toluene (6 mL) was stirred and heated to 100° C. for 2 hours. The mixture was filtered and the filtrate was concentrated in vacuo. The resulting residue was purified using silica gel chromatography (eluting ethyl acetate in petroleum ether) to provide tert-butyl 7-amino-3-bromo-2-((diethoxyphosphoryl)difluoromethyl)benzo[b]thiophene-5-carboxylate. ¹H NMR (300 MHz, chloroform-d) δ 8.07 (d, J=3.7 Hz, 1H), 7.45 (s, 1H), 4.33-4.27 (m, 4H), 1.63 (s, 9H), 1.44-1.32 (m, 6H). MS (ESI, m/z): 514, 516 (M+H)⁺

### Step B: Synthesis of tert-butyl 7-((3-(N,N-bis(4-methoxybenzyl)sulfamoyl)propyl)amino)-3-bromo-2-((diethoxyphosphoryl)difluoromethyl)benzo[b]thiophene-5-carboxylate

A mixture of tert-butyl 7-amino-3-bromo-2-((diethoxyphosphoryl)difluoromethyl)-benzo[b]thiophene-5-carboxylate (120 mg, 0.233 mmol), N,N-bis(4-methoxybenzyl)-3-oxopropane-1-sulfonamide (176 mg, 0.467 mmol), sodium cyanoborohydride (29 mg, 0.47 mmol), and acetic acid (10 μL, 0.18 mmol) in in MeOH (0.2 mL) was allowed to stir for 16 hours at room temperature. The mixture was directly purified using reverse phase HPLC (eluting acetonitrile in water) to provide tert-butyl 7-((3-(N,N-bis(4-methoxybenzyl)sulfamoyl)propyl)amino)-3-bromo-2-((diethoxyphosphoryl)difluoromethyl)benzo[b]thiophene-5-carboxylate. ¹H NMR (300 MHz, CDCl₃) δ 7.99 (s, 1H), 7.28 (s, 1H), 7.25-7.16 (m, 4H), 6.90-6.77 (m, 4H), 4.29 (d, J=5.8 Hz, 8H), 3.78 (s, 6H), 3.52-3.47 (m, 2H), 2.95 (t, J=7.1 Hz, 2H), 2.24-2.11 (m, 2H), 1.64 (s, 9H), 1.36 (t, J=7.1 Hz, 6H). MS (ESI, m/z): 875, 877 (M+H)⁺

### Step C: Synthesis of 3-bromo-2-((diethoxyphosphoryl)difluoromethyl)-7-((3-sulfamoylpropyl)amino)benzo[b]thiophene-5-carboxylic acid

TFA (0.200 mL, 2.60 mmol) was added to a mixture of tert-butyl 7-((3-(N,N-bis(4-methoxybenzyl)sulfamoyl)propyl)amino)-3-bromo-2-((diethoxyphosphoryl)difluoromethyl)-benzo[b]thiophene-5-carboxylate (90 mg, 0.10 mmol) in DCM (1 mL). The resulting reaction was heated to 40° C. for 5 hours. The mixture was concentrated in vacuo to provide 3-bromo-2-((diethoxyphosphoryl)difluoromethyl)-7-((3-sulfamoylpropyl)amino)benzo[b]thiophene-5-carboxylic acid (Int-39). MS (ESI, m/z): 579, 581 (M+H)⁺

### Synthesis of Intermediate Compound Int-40

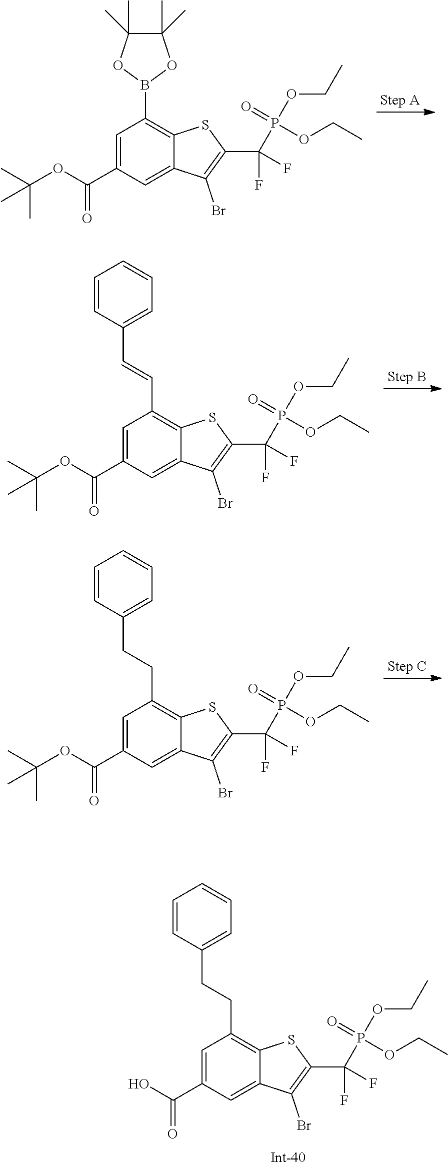

### Step A: Synthesis of tert-butyl (E)-3-bromo-2-((diethoxyphosphoryl)difluoromethyl)-7-styrylbenzo[b]thiophene-5-carboxylate

A mixture of potassium phosphate tribasic (68 mg, 0.32 mmol) in water (267 μl) was added to a mixture of tert-butyl 3-bromo-2-((diethoxyphosphoryl)difluoromethyl)-7-(4,4,5,5-tetramethyl-1,3,2-dioxaborolan-2-yl)benzo[b]thiophene-5-carboxylate (100 mg, 0.160 mmol), (E)-(2-bromovinyl)benzene (44 mg, 0.24 mmol), and cataCXium Pd G4 (30 mg, 0.04 mmol) in THF (1.3 mL). The mixture was heated to 60° C. and stirred under an argon atmosphere for 16 hours. The mixture was diluted with water (25 mL), and washed with ethyl acetate (2×50 mL).

The combined organic layers were dried over anhydrous sodium sulfate, filtered, and concentrated in vacuo. The resulting residue was purified using silica gel chromatography (eluting ethyl acetate in hexanes) to provide tert-butyl (E)-3-bromo-2-((diethoxyphosphoryl)difluoromethyl)-7-styrylbenzo[b]thiophene-5-carboxylate. MS (ESI, m/z): 601, 603 (M+H)⁺

### Step B: Synthesis of tert-butyl 3-bromo-2-((diethoxyphosphoryl)difluoromethyl)-7-phenethylbenzo[b]thiophene-5-carboxylate

A mixture of tert-butyl (E)-3-bromo-2-((diethoxyphosphoryl)difluoromethyl)-7-styrylbenzo[b]thiophene-5-carboxylate (40 mg, 0.067 mmol), and platinum (IV) oxide (23 mg, 0.10 mmol) in EtOH (665 μl) was stirred under a hydrogen atmosphere for 1 hour. The mixture was filtered and concentrated in vacuo to provide tert-butyl 3-bromo-2-((diethoxyphosphoryl)difluoromethyl)-7-phenethylbenzo[b]thiophene-5-carboxylate, which was used in the next step without purification. MS (ESI, m/z): 603, 605 (M+H)⁺

### Step C: Synthesis of 3-bromo-2-((diethoxyphosphoryl)difluoromethyl)-7-phenethylbenzo[b]thiophene-5-carboxylic acid

A mixture of tert-butyl 3-bromo-2-((diethoxyphosphoryl)difluoromethyl)-7-phenethylbenzo[b]thiophene-5-carboxylate (30 mg, 0.050 mmol), and TFA (96 μL, 1.2 mmol) in DCM (0.5 mL) was stirred at room temperature for 2 hours. The mixture was concentrated in vacuo to provide 3-bromo-2-((diethoxyphosphoryl)difluoromethyl)-7-phenethylbenzo[b]thiophene-5-carboxylic acid (Int-40), which was used without purification in the next step. MS (ESI, m/z): 547, 549 (M+H)⁺

The following intermediate compounds were made using the methods described in the Example immediately above, and substituting the appropriate reactants and/or reagents.

| Intermediate | | Mass |
|---|---|---|
| Compound | Structure | [M + H]⁺ |
| Int-40b | 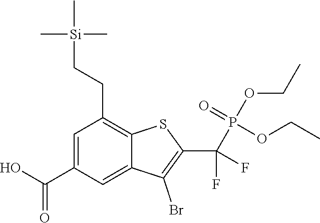 [SMILES: CCOP(=O)(OCC)C(F)(F)c1sc2c(CC[Si](C)(C)C)cc(C(=O)O)cc2c1Br] | 543, 545 |

### Synthesis of Intermediate Compound Int-41

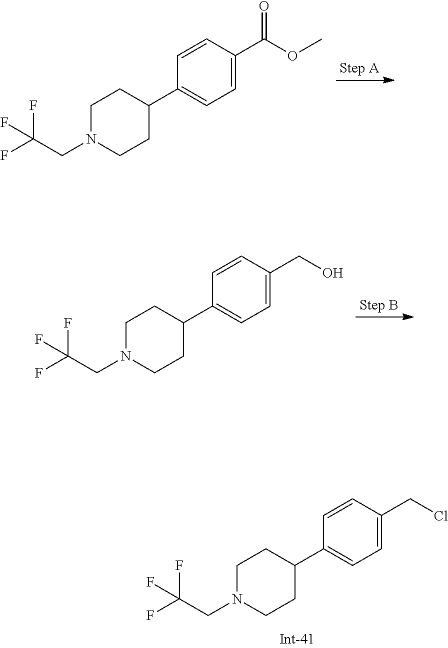 [SMILES: COC(=O)c1ccc(C2CCN(CC(F)(F)F)CC2)cc1 ; OCc1ccc(C2CCN(CC(F)(F)F)CC2)cc1 ; FC(F)(F)CN1CCC(c2ccc(CCl)cc2)CC1]

### Step A: Synthesis of (4-(1-(2,2,2-trifluoroethyl)piperidin-4-yl)phenyl)methanol

To a mixture of methyl 4-(1-(2,2,2-trifluoroethyl)piperidin-4-yl)benzoate (1.55 g, 5.14 mmol) in DCM (15 mL) was added a solution of diisobutylaluminum hydride (1.0 M in THF, 12.9 mL, 13 mmol) dropwise at 5° C. under a nitrogen atmosphere. The mixture was stirred at 5° C. for 30 minutes, and then diluted with Rochelle's salt (1.0 M in water, 30 mL, 30 mmol), and stirred at room temperature for 3 hours. The organic layer was separated, dried over MgSO₄, and concentrated in vacuo to provide (4-(1-(2,2,2-trifluoroethyl)piperidin-4-yl)phenyl)methanol, which was used without purification in the next step. ¹H NMR (400 MHz, CDCl₃) δ 7.32-7.38 (m, 2H), 7.23-7.29 (m, 2H), 4.65-4.72 (m, 2H), 3.00-3.16 (m, 4H), 2.45-2.59 (m, 3H), 1.79-1.90 (m, 4H).

### Step B: Synthesis of 4-(4-(chloromethyl)phenyl)-1-(2,2,2-trifluoroethyl)piperidine

To a mixture of thionyl chloride (0.304 mL, 4.17 mmol) in MeCN (4 mL) was added a solution of (4-(1-(2,2,2-trifluoroethyl)piperidin-4-yl)phenyl)methanol (0.76 g, 2.8 mmol) in MeCN (1 mL) at 5° C. The resulting reaction was allowed to stir at room temperature for 30 minutes, and then was diluted with toluene (10 mL), and concentrated in vacuo to provide 4-(4-(chloromethyl)phenyl)-1-(2,2,2-trifluoroethyl)piperidine (Int-41), which was used without purification in the next step. ¹H NMR (500 MHz, CDCl₃) δ 7.32-7.38 (m, 2H), 7.23-7.28 (m, 2H), 4.57 (s, 2H), 3.9 (br s, 2H), 3.70 (br s, 2H), 3.27 (br s, 2H), 2.74-2.85 (m, 1H), 2.59 (br s, 2H), 1.99-2.09 (m, 2H).

### Synthesis of the Compounds of the Invention

### Example 1

### Preparation of Compound 1

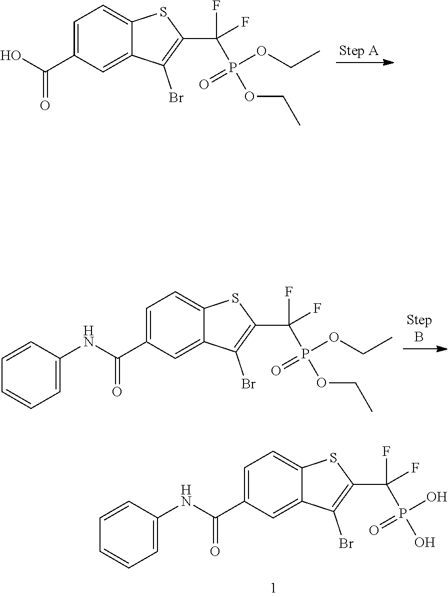

### Step A: Synthesis of diethyl ((3-bromo-5-(phenylcarbamoyl)benzo[b]thiophen-2-yl)difluoromethyl)phosphonate

HATU (172 mg, 0.451 mmol) was added to a mixture of N-ethyl-N-isopropylpropan-2-amine (115 μl, 0.677 mmol)), compound Int-1 (100 mg, 0.226 mmol), and aniline (41 μl, 0.45 mmol) in DMF (1.00 mL) at room temperature. The mixture was stirred overnight at room temperature. The mixture was diluted with EtOAc (5 mL) and washed with brine (3×5 mL). The organic layer was dried over anhydrous sodium sulfate, filtered, and concentrated in vacuo to provide diethyl ((3-bromo-5-(phenylcarbamoyl)benzo[b]thiophen-2-yl)difluoromethyl)phosphonate, which was used in the next step without purification. MS (ESI, m/z): 518, 520 (M+H)⁺

### Step B: Synthesis of ((3-bromo-5-(phenylcarbamoyl)benzo[b]thiophen-2-yl)difluoromethyl)phosphonic acid (Compound 1)

TMS-Br (1.00 mL, 7.71 mmol) was added to a mixture of diethyl ((3-bromo-5-(phenylcarbamoyl)benzo[b]thiophen-2-yl)difluoromethyl)phosphonate (117 mg, 0.226 mmol) in DCM (2 mL). The mixture was allowed to stir at room temperature for 18 hours. The reaction was quenched with methanol (2 mL), stirred for 30 minutes, then concentrated in vacuo and the residue obtained was purified using reverse phase HPLC (eluting acetonitrile in water, with TFA modifier) to provide compound 1. ¹H NMR (499 MHz, DMSO-d₆) δ 10.55 (s, 1H), 8.47 (s, 1H), 8.30 (d, J=8.5 Hz, 1H), 8.14 (d, J=8.4 Hz, 1H), 7.81 (d, J=7.9 Hz, 2H), 7.39 (t, J=7.9 Hz, 2H), 7.14 (t, J=7.4 Hz, 1H). MS (ESI, m/z): 460, 462 (M+H)⁺

The following illustrative compound was made using the methods described in the Example immediately above, and substituting the appropriate reactants and/or reagents.

| | | Mass |
|---|---|---|
| Compound | Structure | [M − H]⁻ |
| 2 | 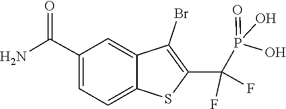 [SMILES: NC(=O)c1ccc2sc(C(F)(F)P(=O)(O)O)c(Br)c2c1] | 384, 386 |

### Example 2

### Preparation of Compound 3

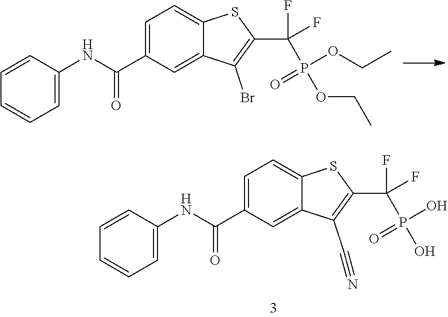

A mixture of diethyl ((3-bromo-5-(phenylcarbamoyl)benzo[b]thiophen-2-yl)difluoromethyl)phosphonate (25 mg, 0.048 mmol), and copper (I) cyanide (22 mg, 0.25 mmol) was evacuated and backfilled with argon (3×). NMP (0.5 mL) was added and the mixture was stirred and heated to 150° C. for 3 hours. The mixture was cooled to room temperature, and TMS-Br (0.50 mL, 3.9 mmol) was added. The mixture was stirred and heated to 60° C. for 4 hours, then quenched with MeOH (3 mL), stirred for 30 minutes, then directly purified using reverse phase HPLC (eluting acetonitrile in water, with TFA modifier) to provide ((3-cyano-5-(phenylcarbamoyl)benzo[b]thiophen-2-yl)difluoromethyl)phosphonic acid (Compound 3). ¹H NMR (499 MHz, DMSO-d₆) δ 10.58 (s, 1H), 8.59 (s, 1H), 8.42 (d, J=8.6 Hz, 1H), 8.20 (d, J=8.6 Hz, 1H), 7.81 (d, J=8.1 Hz, 2H), 7.40 (t, J=7.8 Hz, 2H), 7.15 (t, J=7.4 Hz, 1H). MS (ESI, m/z): 407 (M−H)⁻

The following illustrative compounds were made using the methods described in the Example immediately above, and substituting the appropriate reactants and/or reagents.

| | | Mass |
|---|---|---|
| Compound | Structure | [M + H]⁺ |
| 4 | 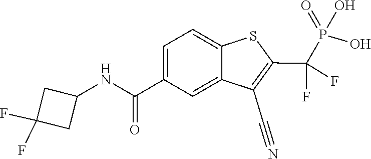 | 423 |
| 5 | 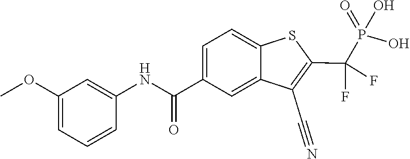 | 439 |
| 6 | 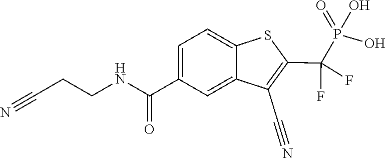 | 386 |

### Example 3

### Preparation of Compound 7

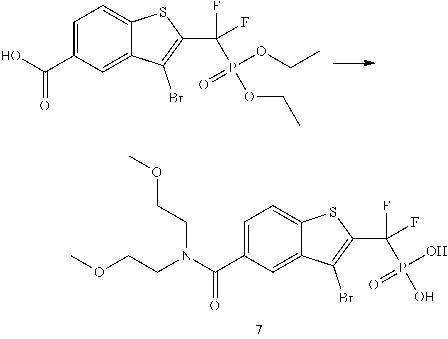

Deoxofluor (81 μl, 0.44 mmol) was added dropwise to a mixture of compound Int-1 (89 mg, 0.02 mmol), acetohydrazide (33 mg, 0.44 mmol), K₂CO₃ (83 mg, 0.60 mmol), and Hünig's base (0.091 mL, 0.52 mmol) in DCM (2.0 mL). The mixture was stirred overnight at room temperature, then the mixture was filtered. The filtrate was concentrated in vacuo, and the resulting residue was diluted with DMF (1.5 mL). TMS-Br (0.20 mL, 1.12 mmol) was added and the mixture was stirred and heated to 50° C. for 12 hours. The reaction was quenched with methanol (2 mL), and directly purified using reverse phase HPLC (eluting acetonitrile in water, with TFA modifier) to provide ((5-(bis(2-methoxyethyl)carbamoyl)-3-bromobenzo[b]thiophen-2-yl)difluoromethyl)phosphonic acid (compound 7). ¹H NMR (600 MHz, DMSO-d₆) δ 8.19 (d, J=8.3 Hz, 1H), 7.90-7.86 (m, 1H), 7.54 (dd, J=8.3, 1.4 Hz, 1H), 3.71-3.64 (m, 2H), 3.63-3.56 (m, 2H), 3.48-3.38 (m, 4H), 3.32 (s, 3H), 3.21 (s, 3H). MS (ESI, m/z): 502, 504 (M+H)⁺

### Example 4

### Preparation of Compound 8

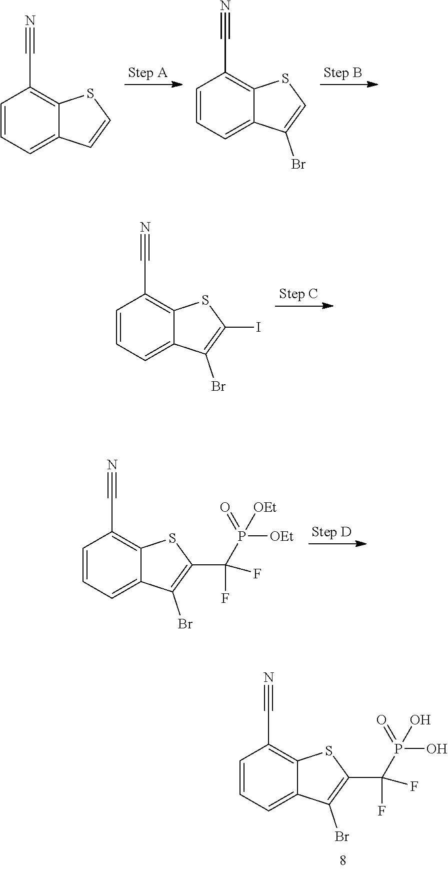 [SMILES: N#Cc1cccc2ccsc12 ; N#Cc1cccc2c(Br)csc12 ; N#Cc1cccc2c(Br)c(I)sc12 ; CCOP(=O)(OCC)C(F)(F)c1sc2c(C#N)cccc2c1Br ; N#Cc1cccc2c(Br)c(C(F)(F)P(=O)(O)O)sc12]

### Step A: Synthesis of 3-bromobenzo[b]thiophene-7-carbonitrile

To a mixture of benzo[b]thiophene-7-carbonitrile (300 mg, 1.9 mmol) in acetic acid (10 mL) was added bromine (0.19 mL, 3.8 mmol). The reaction was allowed to stir for 16 hours at room temperature then concentrated in vacuo. The resulting residue was directly purified using silica gel chromatography (eluting ethyl acetate in petroleum ether) to provide 3-bromobenzo[b]thiophene-7-carbonitrile. ¹H NMR (400 MHz, Chloroform-d) δ 8.06 (dd, J=8.2, 1.1 Hz, 1H), 7.78 (d, J=7.4, 1H), 7.61 (s, 1H), 7.57 (dd, J=8.2, 7.4 Hz, 1H).

### Step B: Synthesis of 3-bromo-2-iodobenzo[b]thiophene-7-carbonitrile

To a mixture of 3-bromobenzo[b]thiophene-7-carbonitrile (50 mg, 0.21 mmol) in THF (1 mL) was added lithium diisopropylamide (2M in THF/heptane/ethylbenzene) (0.22 mL, 0.44 mmol) at −78° C. The mixture was allowed to stir for 1 hour at −78° C. then a solution of iodine (64 mg, 0.25 mmol) in THF (0.5 mL) was added. The mixture was allowed to stir for an additional 2 hours at −78° C. The reaction was quenched by addition of saturated aqueous NH₄Cl (50 mL), and extracted with EtOAc (3×50 mL). The combined organic extracts were washed with brine (50 mL), dried over anhydrous Na₂SO₄ and concentrated in vacuo. The resulting residue was directly purified using silica gel chromatography (eluting ethyl acetate in petroleum ether) to provide 3-bromo-2-iodobenzo[b]thiophene-7-carbonitrile. ¹H NMR (400 MHz, DMSO-d₆) δ 8.15-7.95 (m, 2H), 7.72-7.65 (m, 1H). MS (ESI, m/z): 363, 365 (M+H)⁺

### Step C: Synthesis of diethyl ((3-bromo-7-cyanobenzo[b]thiophen-2-yl)difluoromethyl)phosphonate

To a mixture of cadmium (120 mg, 1.1 mmol) in DMF (1 mL) was added diethyl (bromodifluoromethyl)phosphonate (330 mg, 1.2 mmol), and acetic acid (0.016 mL, 0.27 mmol) under argon. The resulting mixture was allowed to stir for 5 hours at room temperature. This solution was added to a mixture of 3-bromo-2-iodobenzo[b]thiophene-7-carbonitrile (50 mg, 0.14 mmol), and copper(I) chloride (54 mg, 0.55 mmol). The reaction was allowed to stir for 16 hours at room temperature, diluted with water (50 mL), and extracted with EtOAc (3×50 mL). The combined organic extracts were washed with brine (50 mL), dried over anhydrous Na₂SO₄ and concentrated in vacuo. The resulting residue was directly purified using silica gel chromatography (eluting ethyl acetate in petroleum ether) to provide diethyl ((3-bromo-7-cyanobenzo[b]thiophen-2-yl)difluoromethyl)phosphonate. ¹H NMR (400 MHz, Chloroform-d) δ 8.16 (dd, J=8.3, 1.1 Hz, 1H), 7.89-7.82 (m, 1H), 7.62 (dd, J=8.3, 7.4 Hz, 1H), 4.43-4.24 (m, 4H), 1.42-1.40 (m, 6H). MS (ESI, m/z): 424, 426 (M+H)⁺

### Step D: Synthesis of ((3-bromo-7-cyanobenzo[b]thiophen-2-yl)difluoromethyl)phosphonic acid

To a solution of diethyl ((3-bromo-7-cyanobenzo[b]thiophen-2-yl)difluoromethyl)phosphonate (25 mg, 0.059 mmol) in DMF (0.6 mL) was added TMS-Br (0.23 mL, 1.8 mmol) under argon. The resulting solution was allowed to stir for 1.5 hour at 60° C. The reaction was cooled to room temperature, quenched by addition of EtOH (2 mL), and concentrated in vacuo. The resulting residue was purified using reverse phase HPLC (eluting acetonitrile in water with NH₄HCO₃ modifier) to provide ((3-bromo-7-cyanobenzo[b]thiophen-2-yl)difluoromethyl)phosphonic acid (compound 8). ¹H NMR (300 MHz, deuterium oxide) δ 8.20-8.09 (m, 1H), 7.95-7.85 (m, 1H), 7.67-7.55 (m, 1H). MS (ESI, m/z): 366, 368 (M−H)⁻

### Example 5

### Preparation of Compound 9

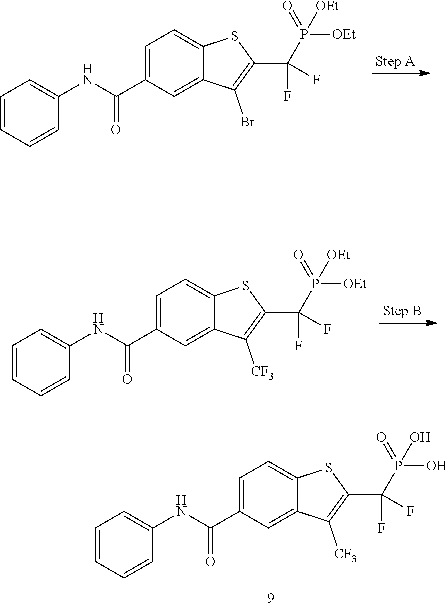 [SMILES: CCOP(=O)(OCC)C(F)(F)c1sc2ccc(C(=O)Nc3ccccc3)cc2c1Br ; CCOP(=O)(OCC)C(F)(F)c1sc2ccc(C(=O)Nc3ccccc3)cc2c1C(F)(F)F ; O=C(Nc1ccccc1)c1ccc2sc(C(F)(F)P(=O)(O)O)c(C(F)(F)F)c2c1]

### Step A: Synthesis of diethyl (difluoro(5-(phenylcarbamoyl)-3-(trifluoromethyl) benzo[b]thiophen-2-yl)methyl)phosphonate

To a mixture of diethyl ((3-bromo-5-(phenylcarbamoyl)benzo[b]thiophen-2-yl)difluoromethyl)phosphonate (50 mg, 0.096 mmol), and copper(I) iodide (3.7 mg, 0.019 mmol) in NMP (0.5 mL) was added methyl 2,2-difluoro-2-(fluorosulfonyl)acetate (56 mg, 0.29 mmol) under argon. The resulting solution was allowed to stir for 16 hours at 80° C. The reaction was cooled to room temperature, and concentrated in vacuo. The resulting residue was directly purified using reverse phase HPLC (eluting acetonitrile in water with TFA modifier) to provide diethyl (difluoro(5-(phenylcarbamoyl)-3-(trifluoromethyl)benzo[b]thiophen-2-yl)methyl)phosphonate. MS (ESI, m/z): 506 (M−H)⁻

### Step B: Synthesis of (difluoro(5-(phenylcarbamoyl)-3-(trifluoromethyl)benzo[b]thiophen-2-yl)methyl)phosphonic acid

To a solution of diethyl (difluoro(5-(phenylcarbamoyl)-3-(trifluoromethyl)benzo[b]thiophen-2-yl)methyl)phosphonate (20 mg, 0.039 mmol) in DMF (0.4 mL) was added TMS-Br (0.15 mL, 1.2 mmol) under argon. The resulting solution was allowed to stir for 1.5 hours at 60° C. The reaction was cooled to room temperature, quenched by addition of EtOH (0.5 mL), and concentrated in vacuo. The resulting residue was purified using reverse phase HPLC (eluting acetonitrile in water with NH₄HCO₃ modifier) to provide (difluoro(5-(phenylcarbamoyl)-3-(trifluoromethyl)benzo[b]thiophen-2-yl)methyl)phosphonic acid (compound 9). ¹H NMR (400 MHz, D₂O) δ 8.52-8.43 (m, 1H), 8.14-8.04 (m, 1H), 7.93-7.83 (m, 1H), 7.56-7.39 (m, 4H), 7.32-7.21 (m, 1H). MS (ESI, m/z): 450 (M−H)⁻

### Example 6

### Preparation of Compound 10

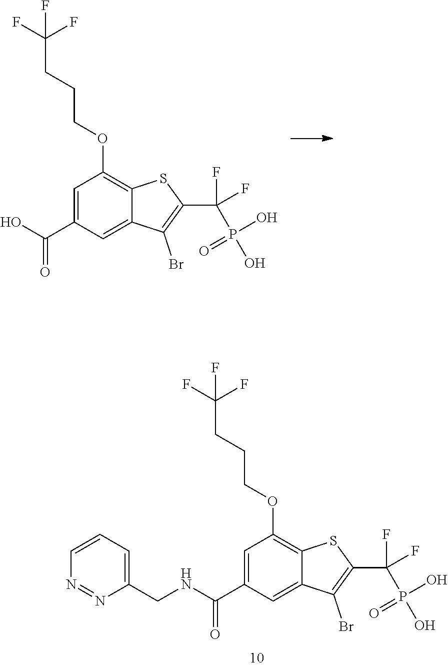 [SMILES: O=C(O)c1cc(OCCCC(F)(F)F)c2sc(C(F)(F)P(=O)(O)O)c(Br)c2c1 ; O=C(NCc1cccnn1)c1cc(OCCCC(F)(F)F)c2sc(C(F)(F)P(=O)(O)O)c(Br)c2c1]

2-(3H-[1,2,3]triazolo[4,5-b]pyridin-3-yl)-1,1,3,3-tetramethylisouronium hexafluorophosphate (V) (14 mg, 0.037 mmol), and Hünig's base (21 μl, 0.12 mmol) were added to a mixture of compound Int-8a (14 mg, 0.025 mmol) in DMF (0.25 mL) at room temperature. The mixture was allowed to stir at room temperature for 5 minutes. Pyridazin-3-ylmethanamine (5 mg, 0.05 mmol) was added, and the resulting reaction was allowed to stir at room temperature for 15 minutes. TMS-Br (96 μl, 0.738 mmol) was added to the mixture. The resulting reaction was heated to 50° C. for 12 hours. The reaction was quenched with MeOH and directly purified using reverse phase HPLC (eluting acetonitrile in water, with TFA modifier) to provide ((3-bromo-5-((pyridazin-3-ylmethyl)carbamoyl)-7-(4,4,4-trifluorobutoxy)benzo[b]thiophen-2-yl)difluoromethyl)phosphonic acid (compound 10). ¹H NMR (600 MHz, DMSO-d₆) δ 9.57 (s, 1H), 9.20-9.14 (m, 1H), 8.12 (s, 1H), 7.75-7.66 (m, 2H), 7.65 (s, 1H), 4.84 (d, J=4.0 Hz, 2H), 4.38 (t, J=5.2 Hz, 2H), 2.51-2.46 (m, 2H), 2.13-2.04 (m, 2H). MS (ESI, m/z): 604 (M+H)⁺

The following illustrative compounds were made using the methods described in the Example immediately above, and substituting the appropriate reactants and/or reagents.

| Com- | | Mass |
|---|---|---|
| pound | Structure | [M + H]⁺ |
| 11 | 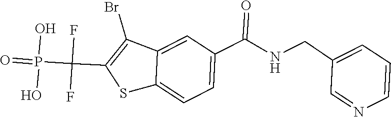 [SMILES: O=C(NCc1cccnc1)c1ccc2sc(C(F)(F)P(=O)(O)O)c(Br)c2c1] | 477, 479 |
| 12 | 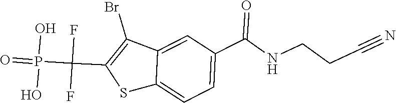 [SMILES: N#CCCNC(=O)c1ccc2sc(C(F)(F)P(=O)(O)O)c(Br)c2c1] | 439, 441 |
| 13 | 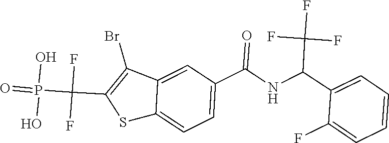 [SMILES: O=C(NC(c1ccccc1F)C(F)(F)F)c1ccc2sc(C(F)(F)P(=O)(O)O)c(Br)c2c1] | 562, 564 |
| 14 | 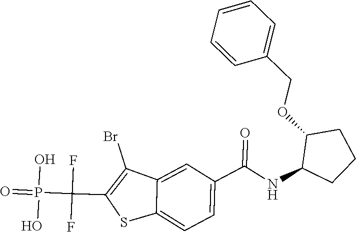 [SMILES: O=C(N[C@@H]1CCC[C@H]1OCc1ccccc1)c1ccc2sc(C(F)(F)P(=O)(O)O)c(Br)c2c1] | 560, 562 |
| 15 | 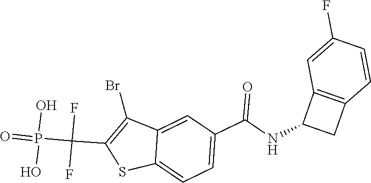 [SMILES: O=C(N[C@H]1Cc2ccc(F)cc21)c1ccc2sc(C(F)(F)P(=O)(O)O)c(Br)c2c1] | 506, 508 |
| 16 | 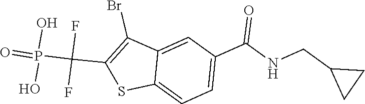 [SMILES: O=C(NCC1CC1)c1ccc2sc(C(F)(F)P(=O)(O)O)c(Br)c2c1] | 440, 442 |
| 17 | 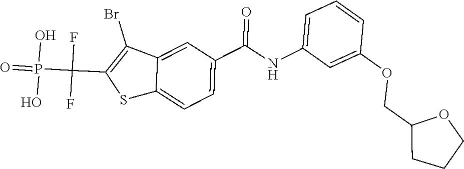 [SMILES: O=C(Nc1cccc(OCC2CCCO2)c1)c1ccc2sc(C(F)(F)P(=O)(O)O)c(Br)c2c1] | 562, 564 |
| 18 | 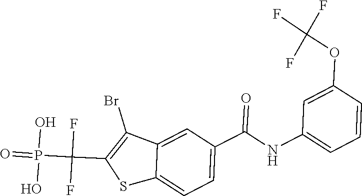 [SMILES: O=C(Nc1cccc(OC(F)(F)F)c1)c1ccc2sc(C(F)(F)P(=O)(O)O)c(Br)c2c1] | 546, 548 |
| 19 | 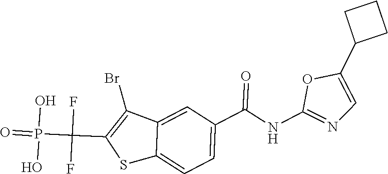 [SMILES: O=C(Nc1ncc(C2CCC2)o1)c1ccc2sc(C(F)(F)P(=O)(O)O)c(Br)c2c1] | 507, 509 |
| 20 | 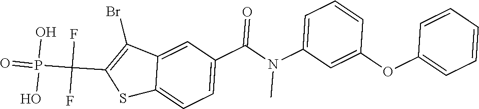 [SMILES: CN(C(=O)c1ccc2sc(C(F)(F)P(=O)(O)O)c(Br)c2c1)c1cccc(Oc2ccccc2)c1] | 568, 570 |
| 21 | 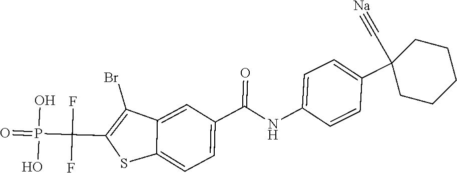 | 569, 571 |
| 22 | 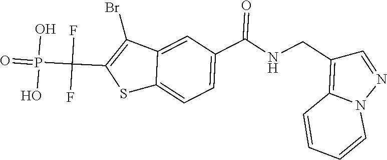 [SMILES: O=C(NCc1cnn2ccccc12)c1ccc2sc(C(F)(F)P(=O)(O)O)c(Br)c2c1] | 516, 518 |
| 23 | 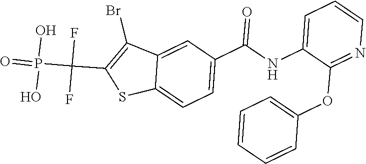 [SMILES: O=C(Nc1cccnc1Oc1ccccc1)c1ccc2sc(C(F)(F)P(=O)(O)O)c(Br)c2c1] | 555, 557 |
| 24 | 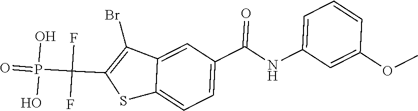 [SMILES: COc1cccc(NC(=O)c2ccc3sc(C(F)(F)P(=O)(O)O)c(Br)c3c2)c1] | 492, 494 |
| 25 | 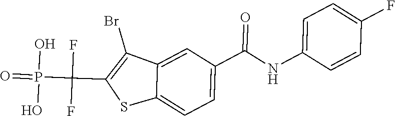 [SMILES: O=C(Nc1ccc(F)cc1)c1ccc2sc(C(F)(F)P(=O)(O)O)c(Br)c2c1] | 480, 482 |
| 26 | 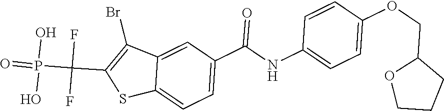 [SMILES: O=C(Nc1ccc(OCC2CCCO2)cc1)c1ccc2sc(C(F)(F)P(=O)(O)O)c(Br)c2c1] | 562, 564 |
| 27 | 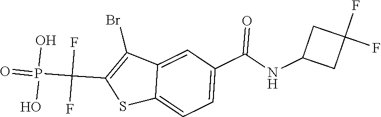 [SMILES: O=C(NC1CC(F)(F)C1)c1ccc2sc(C(F)(F)P(=O)(O)O)c(Br)c2c1] | 476, 478 |
| 28 | 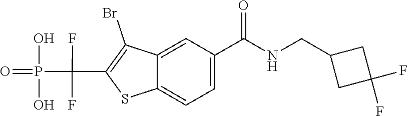 [SMILES: O=C(NCC1CC(F)(F)C1)c1ccc2sc(C(F)(F)P(=O)(O)O)c(Br)c2c1] | 490, 492 |
| 29 | 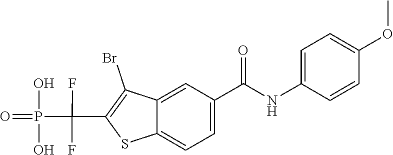 [SMILES: COc1ccc(NC(=O)c2ccc3sc(C(F)(F)P(=O)(O)O)c(Br)c3c2)cc1] | 492, 494 |
| 30 | 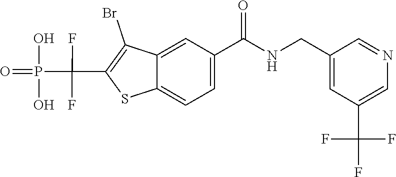 [SMILES: O=C(NCc1cncc(C(F)(F)F)c1)c1ccc2sc(C(F)(F)P(=O)(O)O)c(Br)c2c1] | 545, 547 |
| 31 | 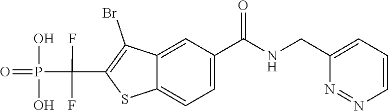 [SMILES: O=C(NCc1cccnn1)c1ccc2sc(C(F)(F)P(=O)(O)O)c(Br)c2c1] | 478, 480 |
| 32 | 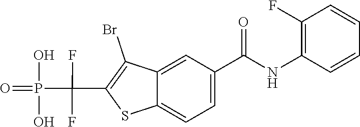 [SMILES: O=C(Nc1ccccc1F)c1ccc2sc(C(F)(F)P(=O)(O)O)c(Br)c2c1] | 480, 482 |
| 33 | 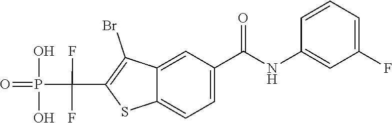 [SMILES: O=C(Nc1cccc(F)c1)c1ccc2sc(C(F)(F)P(=O)(O)O)c(Br)c2c1] | 480, 482 |
| 34 | 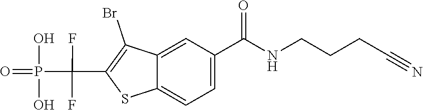 [SMILES: N#CCCCNC(=O)c1ccc2sc(C(F)(F)P(=O)(O)O)c(Br)c2c1] | 453, 455 |
| 35 | 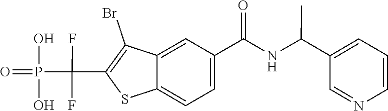 [SMILES: CC(NC(=O)c1ccc2sc(C(F)(F)P(=O)(O)O)c(Br)c2c1)c1cccnc1] | 491, 493 |
| 36 | 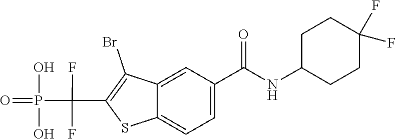 [SMILES: O=C(NC1CCC(F)(F)CC1)c1ccc2sc(C(F)(F)P(=O)(O)O)c(Br)c2c1] | 504, 506 |
| 37 | 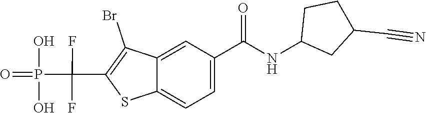 [SMILES: N#CC1CCC(NC(=O)c2ccc3sc(C(F)(F)P(=O)(O)O)c(Br)c3c2)C1] | 479, 481 |
| 38 | 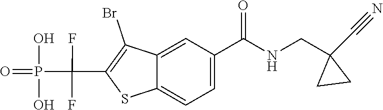 [SMILES: N#CC1(CNC(=O)c2ccc3sc(C(F)(F)P(=O)(O)O)c(Br)c3c2)CC1] | 465, 467 |
| 39 | 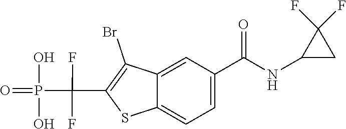 [SMILES: O=C(NC1CC1(F)F)c1ccc2sc(C(F)(F)P(=O)(O)O)c(Br)c2c1] | 462, 464 |
| 40 | 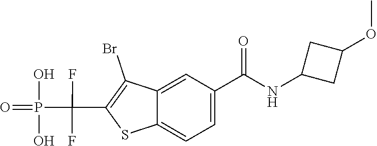 [SMILES: COC1CC(NC(=O)c2ccc3sc(C(F)(F)P(=O)(O)O)c(Br)c3c2)C1] | 470, 472 |
| 41 | 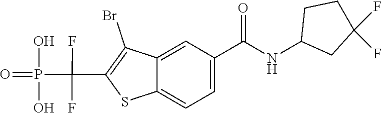 [SMILES: O=C(NC1CCC(F)(F)C1)c1ccc2sc(C(F)(F)P(=O)(O)O)c(Br)c2c1] | 490, 492 |
| 42 | 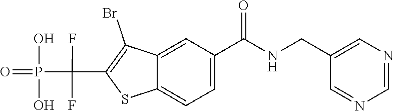 [SMILES: O=C(NCc1cncnc1)c1ccc2sc(C(F)(F)P(=O)(O)O)c(Br)c2c1] | 478, 480 |
| 43 | 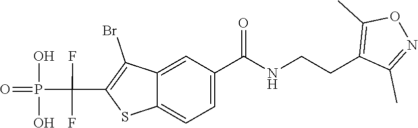 [SMILES: Cc1noc(C)c1CCNC(=O)c1ccc2sc(C(F)(F)P(=O)(O)O)c(Br)c2c1] | 509, 511 |
| 44 | 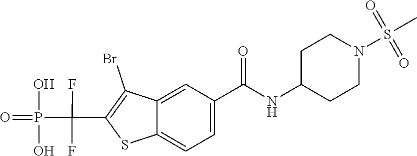 [SMILES: CS(=O)(=O)N1CCC(NC(=O)c2ccc3sc(C(F)(F)P(=O)(O)O)c(Br)c3c2)CC1] | 547, 549 |
| 45 | 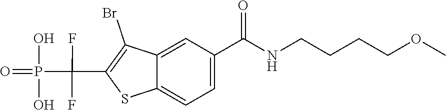 [SMILES: COCCCCNC(=O)c1ccc2sc(C(F)(F)P(=O)(O)O)c(Br)c2c1] | 472, 474 |
| 46 | 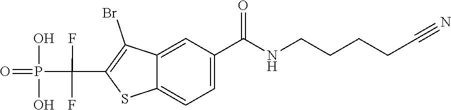 [SMILES: N#CCCCCNC(=O)c1ccc2sc(C(F)(F)P(=O)(O)O)c(Br)c2c1] | 467, 469 |
| 47 | 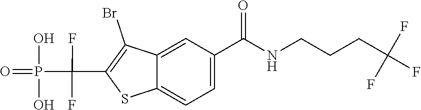 [SMILES: O=C(NCCCC(F)(F)F)c1ccc2sc(C(F)(F)P(=O)(O)O)c(Br)c2c1] | 496, 498 |
| 48 | 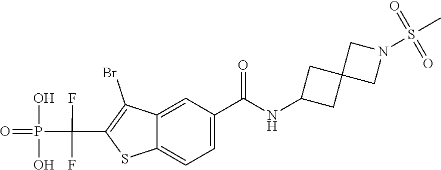 [SMILES: CS(=O)(=O)N1CC2(CC(NC(=O)c3ccc4sc(C(F)(F)P(=O)(O)O)c(Br)c4c3)C2)C1] | 559, 561 |
| 49 | 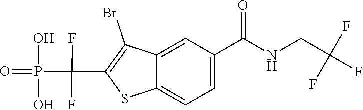 [SMILES: O=C(NCC(F)(F)F)c1ccc2sc(C(F)(F)P(=O)(O)O)c(Br)c2c1] | 468, 470 |
| 50 | 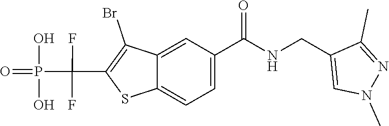 [SMILES: Cc1nn(C)cc1CNC(=O)c1ccc2sc(C(F)(F)P(=O)(O)O)c(Br)c2c1] | 494, 496 |
| 51 | 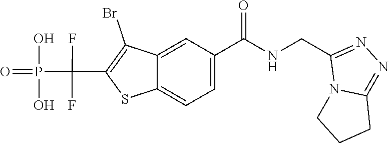 [SMILES: O=C(NCc1nnc2n1CCC2)c1ccc2sc(C(F)(F)P(=O)(O)O)c(Br)c2c1] | 507, 509 |
| 52 | 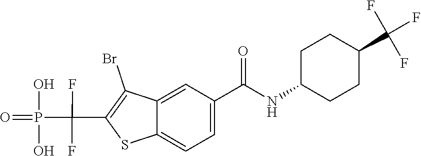 [SMILES: O=C(N[C@H]1CC[C@H](C(F)(F)F)CC1)c1ccc2sc(C(F)(F)P(=O)(O)O)c(Br)c2c1] | 536, 538 |
| 53 | 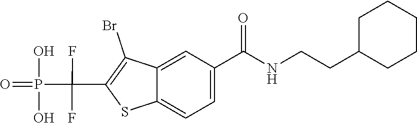 [SMILES: O=C(NCCC1CCCCC1)c1ccc2sc(C(F)(F)P(=O)(O)O)c(Br)c2c1] | 496, 498 |
| 54 | 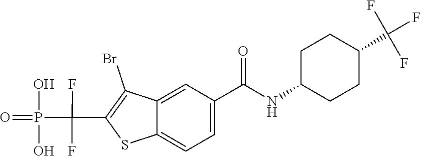 [SMILES: O=C(N[C@H]1CC[C@@H](C(F)(F)F)CC1)c1ccc2sc(C(F)(F)P(=O)(O)O)c(Br)c2c1] | 536, 538 |
| 55 | 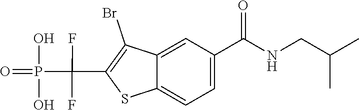 [SMILES: CC(C)CNC(=O)c1ccc2sc(C(F)(F)P(=O)(O)O)c(Br)c2c1] | 442, 444 |
| 56 | 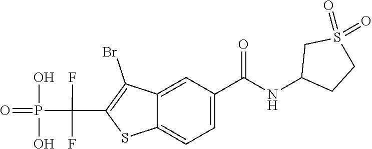 [SMILES: O=C(NC1CCS(=O)(=O)C1)c1ccc2sc(C(F)(F)P(=O)(O)O)c(Br)c2c1] | 504, 506 |
| 57 | 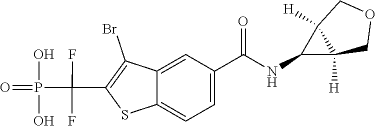 [SMILES: O=C(N[C@@H]1[C@@H]2COC[C@@H]21)c1ccc2sc(C(F)(F)P(=O)(O)O)c(Br)c2c1] | 468, 470 |
| 58 | 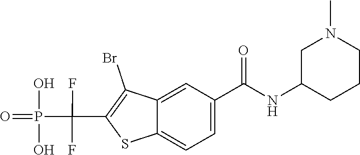 [SMILES: CN1CCCC(NC(=O)c2ccc3sc(C(F)(F)P(=O)(O)O)c(Br)c3c2)C1] | 483, 485 |
| 59 | 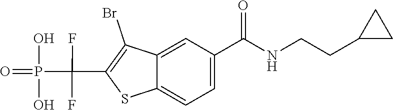 [SMILES: O=C(NCCC1CC1)c1ccc2sc(C(F)(F)P(=O)(O)O)c(Br)c2c1] | 454, 456 |
| 60 | 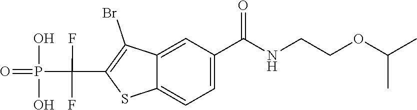 [SMILES: CC(C)OCCNC(=O)c1ccc2sc(C(F)(F)P(=O)(O)O)c(Br)c2c1] | 472, 474 |
| 61 | 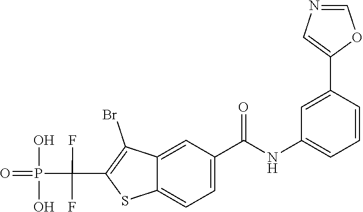 [SMILES: O=C(Nc1cccc(-c2cnco2)c1)c1ccc2sc(C(F)(F)P(=O)(O)O)c(Br)c2c1] | 529, 531 |
| 62 | 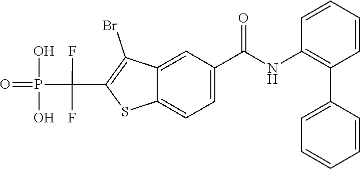 [SMILES: O=C(Nc1ccccc1-c1ccccc1)c1ccc2sc(C(F)(F)P(=O)(O)O)c(Br)c2c1] | 538, 540 |
| 63 | 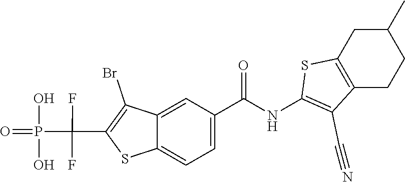 [SMILES: CC1CCc2c(sc(NC(=O)c3ccc4sc(C(F)(F)P(=O)(O)O)c(Br)c4c3)c2C#N)C1] | 561, 563 |
| 64 | 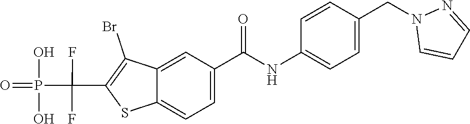 [SMILES: O=C(Nc1ccc(Cn2cccn2)cc1)c1ccc2sc(C(F)(F)P(=O)(O)O)c(Br)c2c1] | 542, 544 |
| 65 | 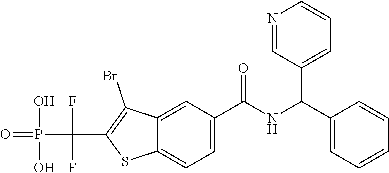 [SMILES: O=C(NC(c1ccccc1)c1cccnc1)c1ccc2sc(C(F)(F)P(=O)(O)O)c(Br)c2c1] | 553, 555 |
| 66 | 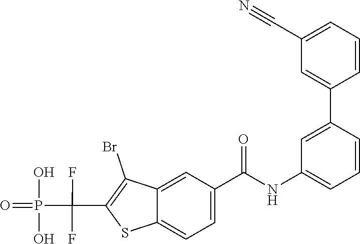 [SMILES: N#Cc1cccc(-c2cccc(NC(=O)c3ccc4sc(C(F)(F)P(=O)(O)O)c(Br)c4c3)c2)c1] | 563, 565 |
| 67 | 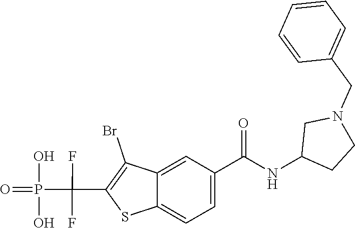 [SMILES: O=C(NC1CCN(Cc2ccccc2)C1)c1ccc2sc(C(F)(F)P(=O)(O)O)c(Br)c2c1] | 545, 547 |
| 68 | 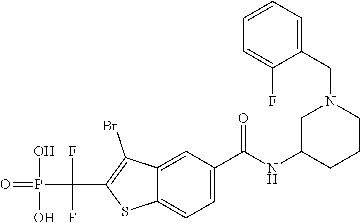 [SMILES: O=C(NC1CCCN(Cc2ccccc2F)C1)c1ccc2sc(C(F)(F)P(=O)(O)O)c(Br)c2c1] | 577, 579 |
| 69 | 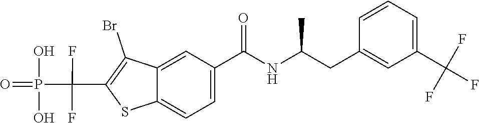 [SMILES: C[C@@H](Cc1cccc(C(F)(F)F)c1)NC(=O)c1ccc2sc(C(F)(F)P(=O)(O)O)c(Br)c2c1] | 572, 574 |
| 70 | 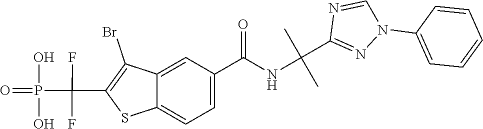 [SMILES: CC(C)(NC(=O)c1ccc2sc(C(F)(F)P(=O)(O)O)c(Br)c2c1)c1ncn(-c2ccccc2)n1] | 571, 573 |
| 71 | 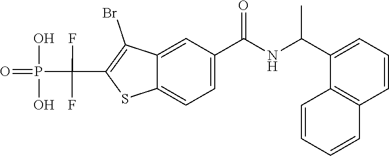 [SMILES: CC(NC(=O)c1ccc2sc(C(F)(F)P(=O)(O)O)c(Br)c2c1)c1cccc2ccccc12] | 540, 542 |
| 72 | 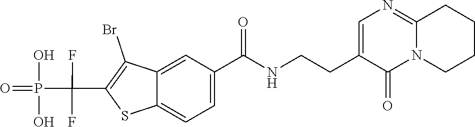 [SMILES: O=C(NCCc1cnc2n(c1=O)CCCC2)c1ccc2sc(C(F)(F)P(=O)(O)O)c(Br)c2c1] | 562, 564 |
| 73 | 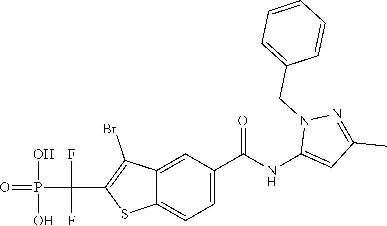 [SMILES: Cc1cc(NC(=O)c2ccc3sc(C(F)(F)P(=O)(O)O)c(Br)c3c2)n(Cc2ccccc2)n1] | 556, 558 |
| 74 | 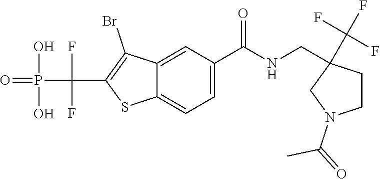 [SMILES: CC(=O)N1CCC(CNC(=O)c2ccc3sc(C(F)(F)P(=O)(O)O)c(Br)c3c2)(C(F)(F)F)C1] | 579, 581 |
| 75 | 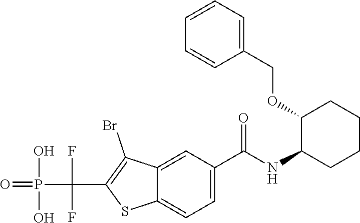 [SMILES: O=C(N[C@@H]1CCCC[C@H]1OCc1ccccc1)c1ccc2sc(C(F)(F)P(=O)(O)O)c(Br)c2c1] | 574, 576 |
| 76 | 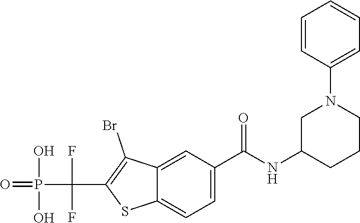 [SMILES: O=C(NC1CCCN(c2ccccc2)C1)c1ccc2sc(C(F)(F)P(=O)(O)O)c(Br)c2c1] | 545, 547 |
| 77 | 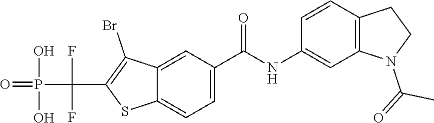 [SMILES: CC(=O)N1CCc2ccc(NC(=O)c3ccc4sc(C(F)(F)P(=O)(O)O)c(Br)c4c3)cc21] | 545, 547 |
| 78 | 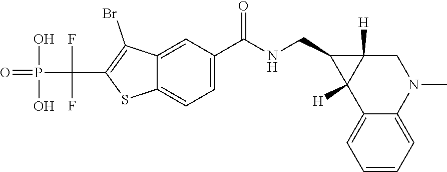 [SMILES: CN1C[C@H]2[C@H](CNC(=O)c3ccc4sc(C(F)(F)P(=O)(O)O)c(Br)c4c3)[C@H]2c2ccccc21] | 557, 559 |
| 79 | 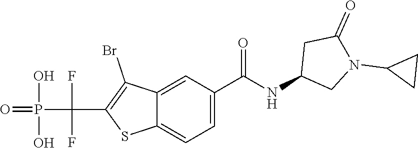 [SMILES: O=C(N[C@H]1CC(=O)N(C2CC2)C1)c1ccc2sc(C(F)(F)P(=O)(O)O)c(Br)c2c1] | 509, 511 |
| 80 | 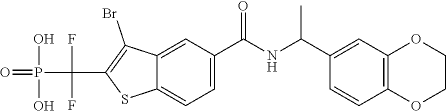 [SMILES: CC(NC(=O)c1ccc2sc(C(F)(F)P(=O)(O)O)c(Br)c2c1)c1ccc2c(c1)OCCO2] | 548, 550 |
| 81 | 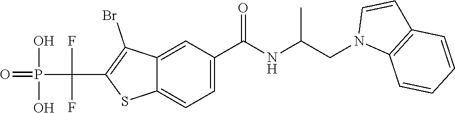 [SMILES: CC(Cn1ccc2ccccc21)NC(=O)c1ccc2sc(C(F)(F)P(=O)(O)O)c(Br)c2c1] | 543, 545 |
| 82 | 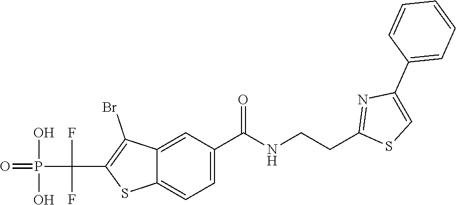 [SMILES: O=C(NCCc1nc(-c2ccccc2)cs1)c1ccc2sc(C(F)(F)P(=O)(O)O)c(Br)c2c1] | 573, 575 |
| 83 | 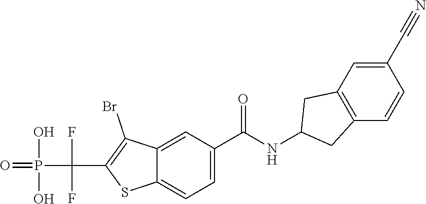 [SMILES: N#Cc1ccc2c(c1)CC(NC(=O)c1ccc3sc(C(F)(F)P(=O)(O)O)c(Br)c3c1)C2] | 527, 529 |
| 84 | 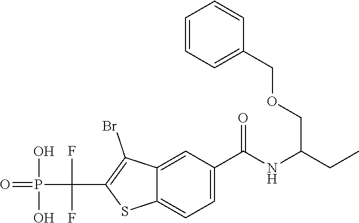 [SMILES: CCC(COCc1ccccc1)NC(=O)c1ccc2sc(C(F)(F)P(=O)(O)O)c(Br)c2c1] | 548, 550 |
| 85 | 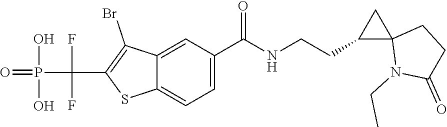 [SMILES: CCN1C(=O)CCC12C[C@H]2CCNC(=O)c1ccc2sc(C(F)(F)P(=O)(O)O)c(Br)c2c1] | 551, 553 |
| 86 | 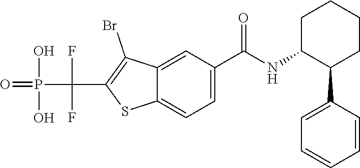 [SMILES: O=C(N[C@@H]1CCCC[C@H]1c1ccccc1)c1ccc2sc(C(F)(F)P(=O)(O)O)c(Br)c2c1] | 544, 546 |
| 87 | 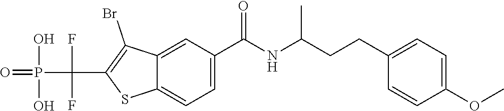 [SMILES: COc1ccc(CCC(C)NC(=O)c2ccc3sc(C(F)(F)P(=O)(O)O)c(Br)c3c2)cc1] | 548, 550 |
| 88 | 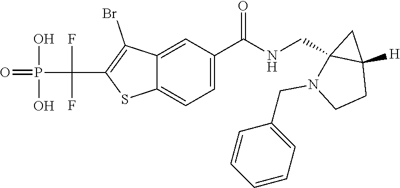 [SMILES: O=C(NC[C@]12C[C@@H]1CCN2Cc1ccccc1)c1ccc2sc(C(F)(F)P(=O)(O)O)c(Br)c2c1] | 571, 573 |
| 89 | 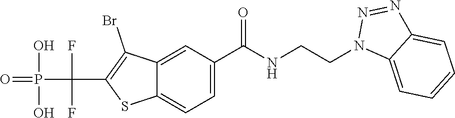 [SMILES: O=C(NCCn1nnc2ccccc21)c1ccc2sc(C(F)(F)P(=O)(O)O)c(Br)c2c1] | 531, 533 |
| 90 | 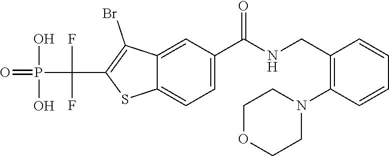 [SMILES: O=C(NCc1ccccc1N1CCOCC1)c1ccc2sc(C(F)(F)P(=O)(O)O)c(Br)c2c1] | 561, 563 |
| 91 | 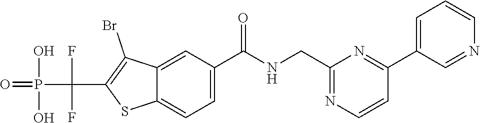 [SMILES: O=C(NCc1nccc(-c2cccnc2)n1)c1ccc2sc(C(F)(F)P(=O)(O)O)c(Br)c2c1] | 555, 557 |
| 92 | 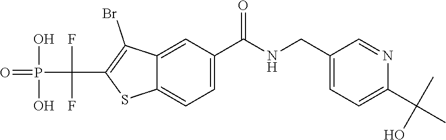 [SMILES: CC(C)(O)c1ccc(CNC(=O)c2ccc3sc(C(F)(F)P(=O)(O)O)c(Br)c3c2)cn1] | 535, 537 |
| 93 | 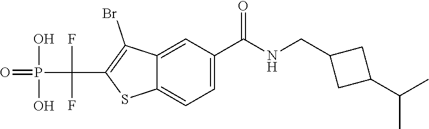 [SMILES: CC(C)C1CC(CNC(=O)c2ccc3sc(C(F)(F)P(=O)(O)O)c(Br)c3c2)C1] | 496, 498 |
| 94 | 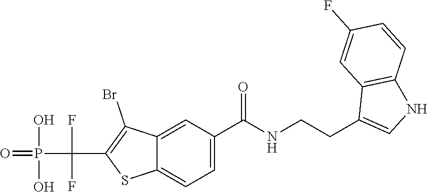 [SMILES: O=C(NCCc1c[nH]c2ccc(F)cc12)c1ccc2sc(C(F)(F)P(=O)(O)O)c(Br)c2c1] | 547, 549 |
| 95 | 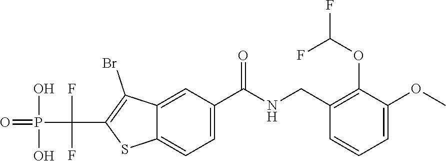 [SMILES: COc1cccc(CNC(=O)c2ccc3sc(C(F)(F)P(=O)(O)O)c(Br)c3c2)c1OC(F)F] | 572, 574 |
| 96 | 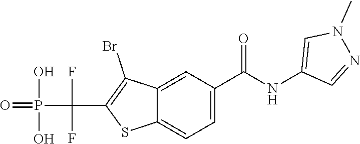 [SMILES: Cn1cc(NC(=O)c2ccc3sc(C(F)(F)P(=O)(O)O)c(Br)c3c2)cn1] | 466, 468 |
| 97 | 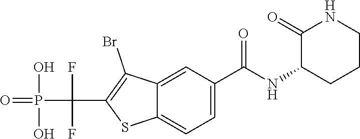 [SMILES: O=C(N[C@H]1CCCNC1=O)c1ccc2sc(C(F)(F)P(=O)(O)O)c(Br)c2c1] | 483, 485 |
| 98 | 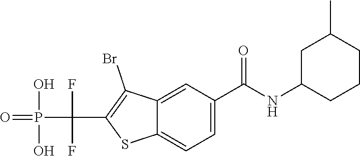 [SMILES: CC1CCCC(NC(=O)c2ccc3sc(C(F)(F)P(=O)(O)O)c(Br)c3c2)C1] | 482, 484 |
| 99 | 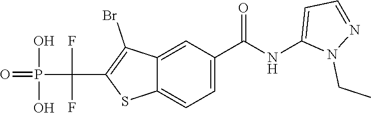 [SMILES: CCn1nccc1NC(=O)c1ccc2sc(C(F)(F)P(=O)(O)O)c(Br)c2c1] | 480, 482 |
| 100 | 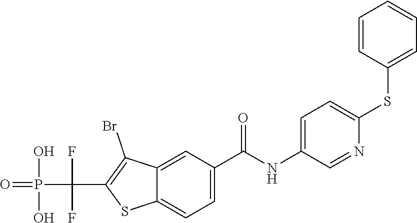 [SMILES: O=C(Nc1ccc(Sc2ccccc2)nc1)c1ccc2sc(C(F)(F)P(=O)(O)O)c(Br)c2c1] | 571, 573 |
| 101 | 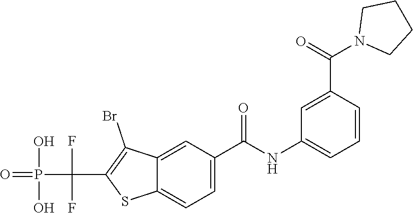 [SMILES: O=C(Nc1cccc(C(=O)N2CCCC2)c1)c1ccc2sc(C(F)(F)P(=O)(O)O)c(Br)c2c1] | 559, 561 |
| 102 | 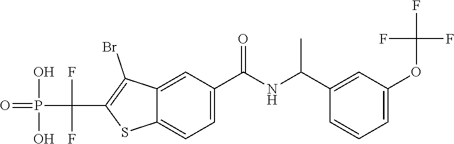 [SMILES: CC(NC(=O)c1ccc2sc(C(F)(F)P(=O)(O)O)c(Br)c2c1)c1cccc(OC(F)(F)F)c1] | 574, 576 |
| 103 | 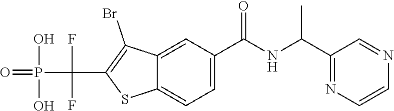 [SMILES: CC(NC(=O)c1ccc2sc(C(F)(F)P(=O)(O)O)c(Br)c2c1)c1cnccn1] | 492, 494 |
| 104 | 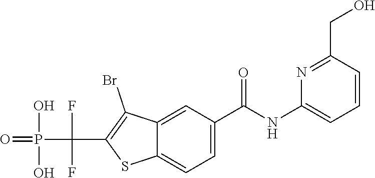 [SMILES: O=C(Nc1cccc(CO)n1)c1ccc2sc(C(F)(F)P(=O)(O)O)c(Br)c2c1] | 493, 495 |
| 105 | 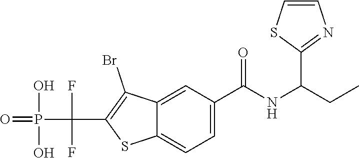 [SMILES: CCC(NC(=O)c1ccc2sc(C(F)(F)P(=O)(O)O)c(Br)c2c1)c1nccs1] | 511, 513 |
| 106 | 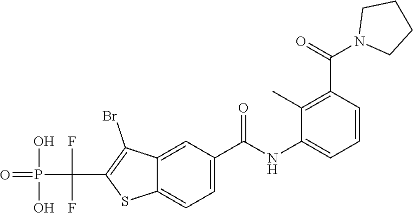 [SMILES: Cc1c(NC(=O)c2ccc3sc(C(F)(F)P(=O)(O)O)c(Br)c3c2)cccc1C(=O)N1CCCC1] | 573, 575 |
| 107 | 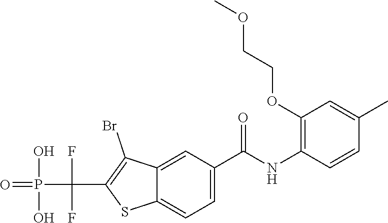 [SMILES: COCCOc1cc(C)ccc1NC(=O)c1ccc2sc(C(F)(F)P(=O)(O)O)c(Br)c2c1] | 550, 552 |
| 108 | 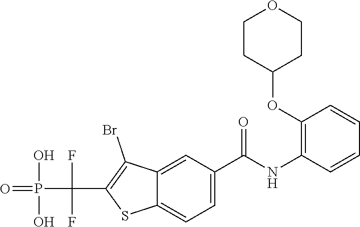 [SMILES: O=C(Nc1ccccc1OC1CCOCC1)c1ccc2sc(C(F)(F)P(=O)(O)O)c(Br)c2c1] | 562, 564 |
| 109 | 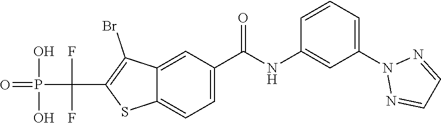 [SMILES: O=C(Nc1cccc(-n2nccn2)c1)c1ccc2sc(C(F)(F)P(=O)(O)O)c(Br)c2c1] | 529, 531 |
| 110 | 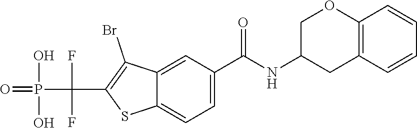 [SMILES: O=C(NC1COc2ccccc2C1)c1ccc2sc(C(F)(F)P(=O)(O)O)c(Br)c2c1] | 518, 520 |
| 111 | 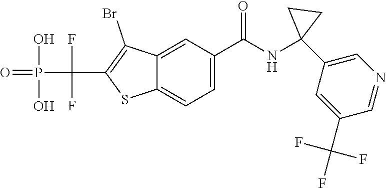 [SMILES: O=C(NC1(c2cncc(C(F)(F)F)c2)CC1)c1ccc2sc(C(F)(F)P(=O)(O)O)c(Br)c2c1] | 571, 573 |
| 112 | 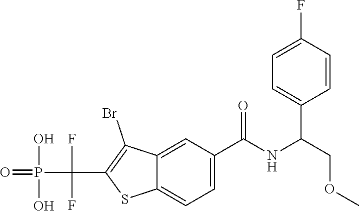 [SMILES: COCC(NC(=O)c1ccc2sc(C(F)(F)P(=O)(O)O)c(Br)c2c1)c1ccc(F)cc1] | 538, 540 |
| 113 | 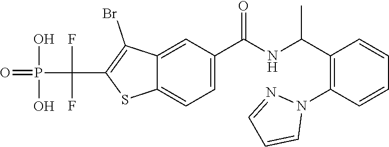 [SMILES: CC(NC(=O)c1ccc2sc(C(F)(F)P(=O)(O)O)c(Br)c2c1)c1ccccc1-n1cccn1] | 556, 558 |
| 114 | 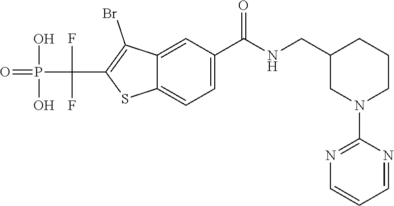 [SMILES: O=C(NCC1CCCN(c2ncccn2)C1)c1ccc2sc(C(F)(F)P(=O)(O)O)c(Br)c2c1] | 561, 563 |
| 115 | 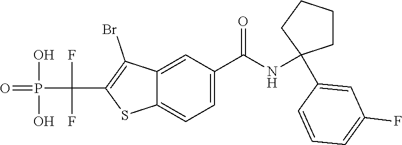 [SMILES: O=C(NC1(c2cccc(F)c2)CCCC1)c1ccc2sc(C(F)(F)P(=O)(O)O)c(Br)c2c1] | 548, 550 |
| 116 | 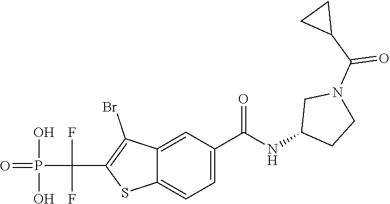 [SMILES: O=C(N[C@H]1CCN(C(=O)C2CC2)C1)c1ccc2sc(C(F)(F)P(=O)(O)O)c(Br)c2c1] | 523, 525 |
| 117 | 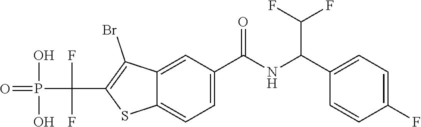 [SMILES: O=C(NC(c1ccc(F)cc1)C(F)F)c1ccc2sc(C(F)(F)P(=O)(O)O)c(Br)c2c1] | 544, 546 |
| 118 | 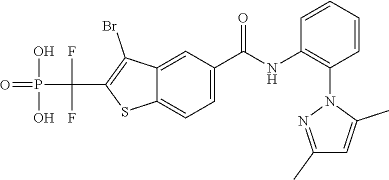 [SMILES: Cc1cc(C)n(-c2ccccc2NC(=O)c2ccc3sc(C(F)(F)P(=O)(O)O)c(Br)c3c2)n1] | 556, 558 |
| 119 | 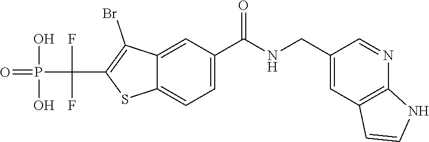 [SMILES: O=C(NCc1cnc2[nH]ccc2c1)c1ccc2sc(C(F)(F)P(=O)(O)O)c(Br)c2c1] | 516, 518 |
| 120 | 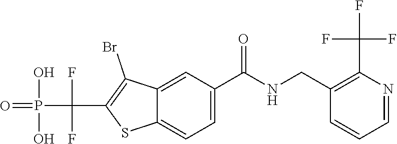 [SMILES: O=C(NCc1cccnc1C(F)(F)F)c1ccc2sc(C(F)(F)P(=O)(O)O)c(Br)c2c1] | 545, 547 |
| 121 | 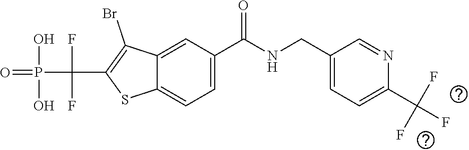 [SMILES: O=C(NCc1ccc(C(F)(F)F)nc1)c1ccc2sc(C(F)(F)P(=O)(O)O)c(Br)c2c1] | 545, 547 |
| 122 | 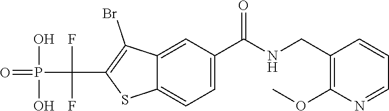 [SMILES: COc1ncccc1CNC(=O)c1ccc2sc(C(F)(F)P(=O)(O)O)c(Br)c2c1] | 507, 509 |
| 123 | 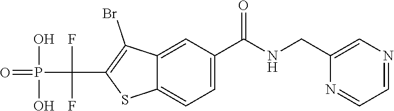 [SMILES: O=C(NCc1cnccn1)c1ccc2sc(C(F)(F)P(=O)(O)O)c(Br)c2c1] | 478, 480 |
| 124 | 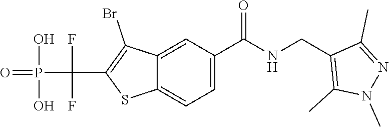 [SMILES: Cc1nn(C)c(C)c1CNC(=O)c1ccc2sc(C(F)(F)P(=O)(O)O)c(Br)c2c1] | 508, 510 |
| 125 | 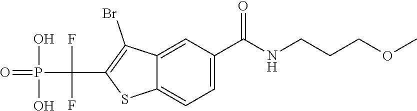 [SMILES: COCCCNC(=O)c1ccc2sc(C(F)(F)P(=O)(O)O)c(Br)c2c1] | 458, 460 |
| 126 | 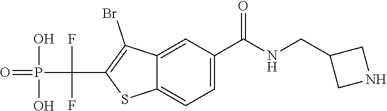 [SMILES: O=C(NCC1CNC1)c1ccc2sc(C(F)(F)P(=O)(O)O)c(Br)c2c1] | 455, 457 |
| 127 | 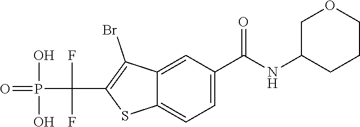 [SMILES: O=C(NC1CCCOC1)c1ccc2sc(C(F)(F)P(=O)(O)O)c(Br)c2c1] | 470, 472 |
| 128 | 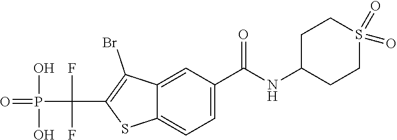 [SMILES: O=C(NC1CCS(=O)(=O)CC1)c1ccc2sc(C(F)(F)P(=O)(O)O)c(Br)c2c1] | 518, 520 |
| 129 | 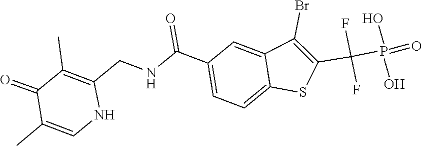 [SMILES: Cc1c[nH]c(CNC(=O)c2ccc3sc(C(F)(F)P(=O)(O)O)c(Br)c3c2)c(C)c1=O] | 521, 523 |
| 130 | 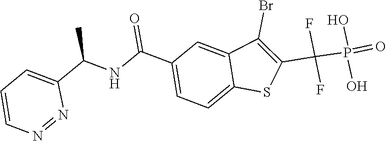 [SMILES: C[C@@H](NC(=O)c1ccc2sc(C(F)(F)P(=O)(O)O)c(Br)c2c1)c1cccnn1] | 492, 494 |
| 131 | 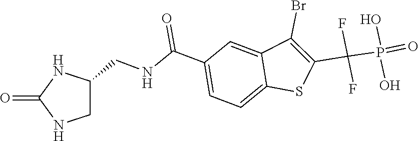 [SMILES: O=C1NC[C@@H](CNC(=O)c2ccc3sc(C(F)(F)P(=O)(O)O)c(Br)c3c2)N1] | 484, 486 |
| 132 | 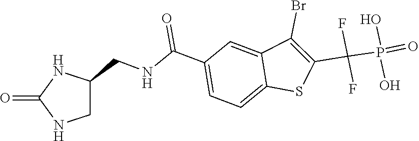 [SMILES: O=C1NC[C@H](CNC(=O)c2ccc3sc(C(F)(F)P(=O)(O)O)c(Br)c3c2)N1] | 484, 486 |
| 133 | 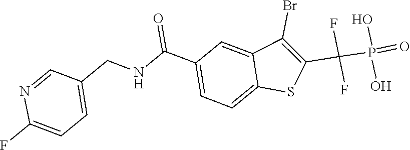 [SMILES: O=C(NCc1ccc(F)nc1)c1ccc2sc(C(F)(F)P(=O)(O)O)c(Br)c2c1] | 495, 497 |
| 134 | 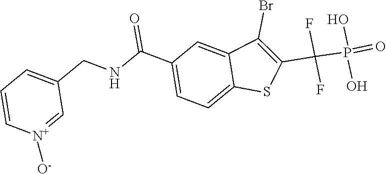 [SMILES: O=C(NCc1ccc[n+]([O-])c1)c1ccc2sc(C(F)(F)P(=O)(O)O)c(Br)c2c1] | 493, 495 |
| 135 | 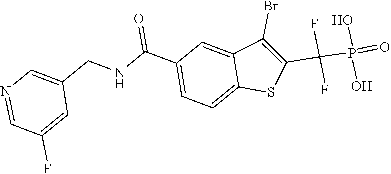 [SMILES: O=C(NCc1cncc(F)c1)c1ccc2sc(C(F)(F)P(=O)(O)O)c(Br)c2c1] | 495, 497 |
| 136 | 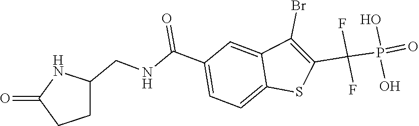 [SMILES: O=C1CCC(CNC(=O)c2ccc3sc(C(F)(F)P(=O)(O)O)c(Br)c3c2)N1] | 483, 485 |
| 137 | 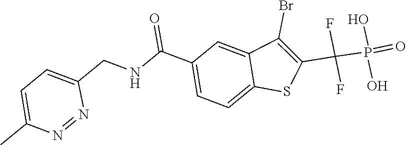 [SMILES: Cc1ccc(CNC(=O)c2ccc3sc(C(F)(F)P(=O)(O)O)c(Br)c3c2)nn1] | 492, 494 |
| 138 | 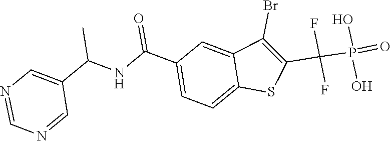 [SMILES: CC(NC(=O)c1ccc2sc(C(F)(F)P(=O)(O)O)c(Br)c2c1)c1cncnc1] | 492, 494 |
| 139 | 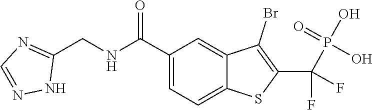 [SMILES: O=C(NCc1ncn[nH]1)c1ccc2sc(C(F)(F)P(=O)(O)O)c(Br)c2c1] | 467, 469 |
| 140 | 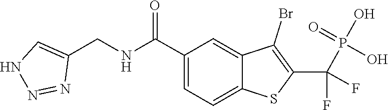 [SMILES: O=C(NCc1c[nH]nn1)c1ccc2sc(C(F)(F)P(=O)(O)O)c(Br)c2c1] | 467, 469 |
| 141 | 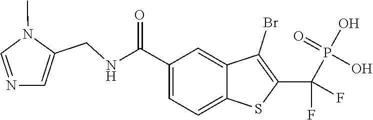 [SMILES: Cn1cncc1CNC(=O)c1ccc2sc(C(F)(F)P(=O)(O)O)c(Br)c2c1] | 480, 482 |
| 142 | 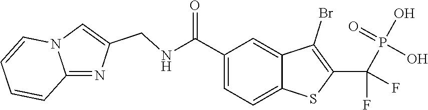 [SMILES: O=C(NCc1cn2ccccc2n1)c1ccc2sc(C(F)(F)P(=O)(O)O)c(Br)c2c1] | 516, 518 |
| 143 | 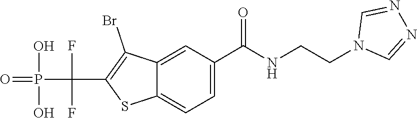 [SMILES: O=C(NCCn1cnnc1)c1ccc2sc(C(F)(F)P(=O)(O)O)c(Br)c2c1] | 481, 483 |
| 144 | 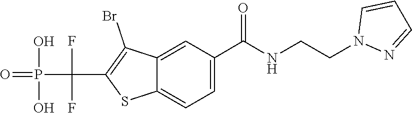 [SMILES: O=C(NCCn1cccn1)c1ccc2sc(C(F)(F)P(=O)(O)O)c(Br)c2c1] | 480, 482 |
| 145 | 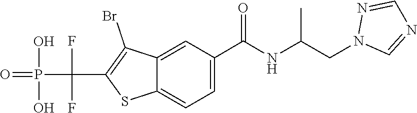 [SMILES: CC(Cn1cncn1)NC(=O)c1ccc2sc(C(F)(F)P(=O)(O)O)c(Br)c2c1] | 495, 497 |
| 146 | 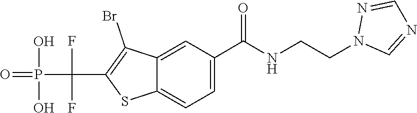 [SMILES: O=C(NCCn1cncn1)c1ccc2sc(C(F)(F)P(=O)(O)O)c(Br)c2c1] | 481, 483 |
| 147 | 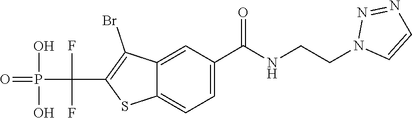 [SMILES: O=C(NCCn1ccnn1)c1ccc2sc(C(F)(F)P(=O)(O)O)c(Br)c2c1] | 481, 483 |
| 148 | 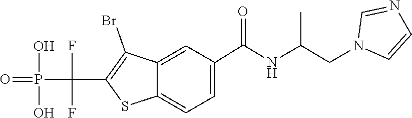 [SMILES: CC(Cn1ccnc1)NC(=O)c1ccc2sc(C(F)(F)P(=O)(O)O)c(Br)c2c1] | 494, 496 |
| 149 | 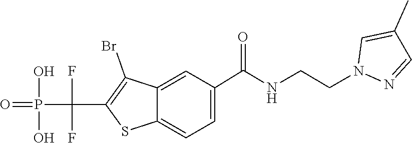 [SMILES: Cc1cnn(CCNC(=O)c2ccc3sc(C(F)(F)P(=O)(O)O)c(Br)c3c2)c1] | 494, 496 |
| 150 | 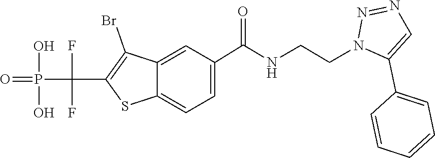 [SMILES: O=C(NCCn1nncc1-c1ccccc1)c1ccc2sc(C(F)(F)P(=O)(O)O)c(Br)c2c1] | 557, 559 |
| 151 | 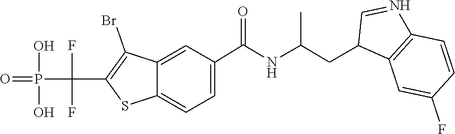 [SMILES: CC(CC1C=Nc2ccc(F)cc21)NC(=O)c1ccc2sc(C(F)(F)P(=O)(O)O)c(Br)c2c1] | 561, 563 |
| 152 | 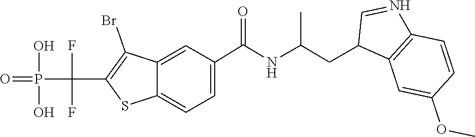 [SMILES: COc1ccc2c(c1)C(CC(C)NC(=O)c1ccc3sc(C(F)(F)P(=O)(O)O)c(Br)c3c1)C=N2] | 573, 575 |
| 153 | 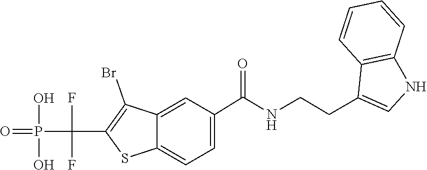 [SMILES: O=C(NCCc1c[nH]c2ccccc12)c1ccc2sc(C(F)(F)P(=O)(O)O)c(Br)c2c1] | 529, 531 |
| 154 | 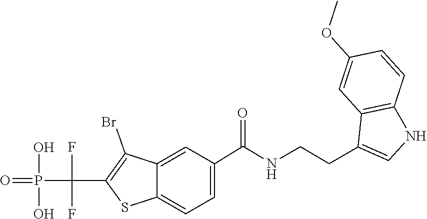 [SMILES: COc1ccc2[nH]cc(CCNC(=O)c3ccc4sc(C(F)(F)P(=O)(O)O)c(Br)c4c3)c2c1] | 559, 561 |
| 155 | 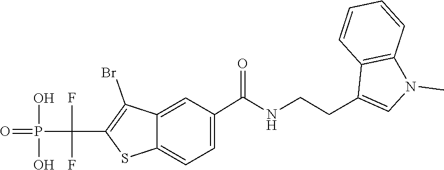 [SMILES: Cn1cc(CCNC(=O)c2ccc3sc(C(F)(F)P(=O)(O)O)c(Br)c3c2)c2ccccc21] | 543, 545 |
| 156 | 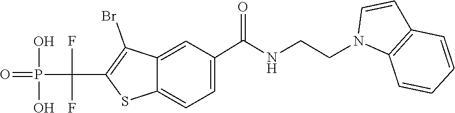 [SMILES: O=C(NCCn1ccc2ccccc21)c1ccc2sc(C(F)(F)P(=O)(O)O)c(Br)c2c1] | 529, 531 |
| 157 | 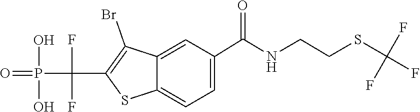 [SMILES: O=C(NCCSC(F)(F)F)c1ccc2sc(C(F)(F)P(=O)(O)O)c(Br)c2c1] | 514, 516 |
| 158 | 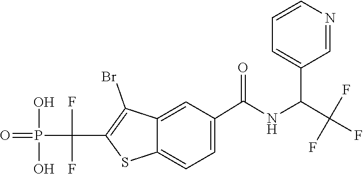 [SMILES: O=C(NC(c1cccnc1)C(F)(F)F)c1ccc2sc(C(F)(F)P(=O)(O)O)c(Br)c2c1] | 545, 547 |
| 159 | 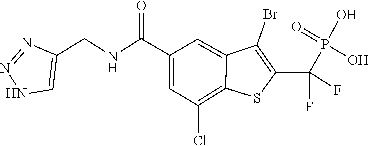 [SMILES: O=C(NCc1c[nH]nn1)c1cc(Cl)c2sc(C(F)(F)P(=O)(O)O)c(Br)c2c1] | 501, 503 |
| 160 | 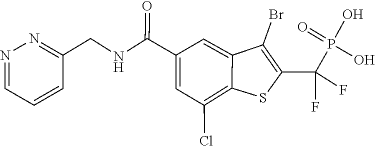 [SMILES: O=C(NCc1cccnn1)c1cc(Cl)c2sc(C(F)(F)P(=O)(O)O)c(Br)c2c1] | 512, 514 |
| 161 | 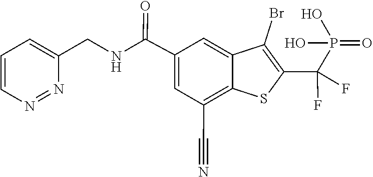 | 503, 505 |
| 162 | 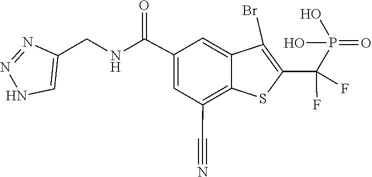 | 492, 494 |
| 163 | 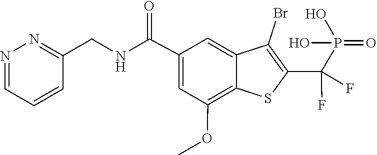 | 508, 510 |
| 164 | 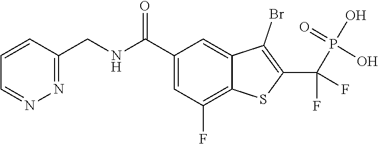 [SMILES: O=C(NCc1cccnn1)c1cc(F)c2sc(C(F)(F)P(=O)(O)O)c(Br)c2c1] | 496, 498 |
| 165 | 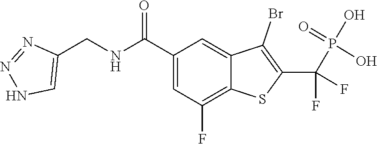 [SMILES: O=C(NCc1c[nH]nn1)c1cc(F)c2sc(C(F)(F)P(=O)(O)O)c(Br)c2c1] | 485, 487 |
| 166 | 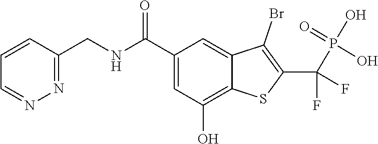 [SMILES: O=C(NCc1cccnn1)c1cc(O)c2sc(C(F)(F)P(=O)(O)O)c(Br)c2c1] | 494, 496 |
| 167 | 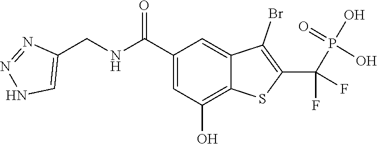 [SMILES: O=C(NCc1c[nH]nn1)c1cc(O)c2sc(C(F)(F)P(=O)(O)O)c(Br)c2c1] | 483, 485 |
| 168 | 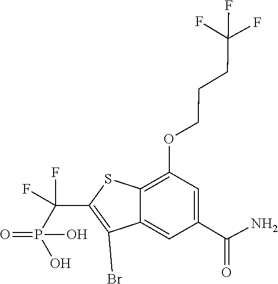 [SMILES: NC(=O)c1cc(OCCCC(F)(F)F)c2sc(C(F)(F)P(=O)(O)O)c(Br)c2c1] | 512, 514 |
| 169 | 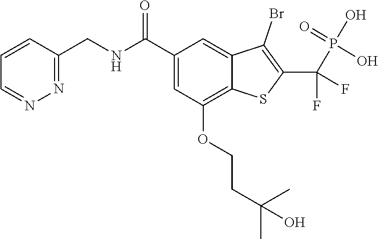 [SMILES: CC(C)(O)CCOc1cc(C(=O)NCc2cccnn2)cc2c(Br)c(C(F)(F)P(=O)(O)O)sc12] | 580, 582 |
| 170 | 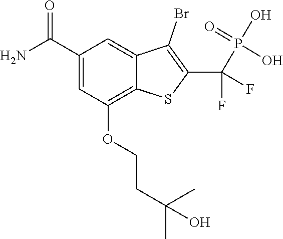 [SMILES: CC(C)(O)CCOc1cc(C(N)=O)cc2c(Br)c(C(F)(F)P(=O)(O)O)sc12] | 470, 472 [M + H − H₂O]⁺ |
| 171 | 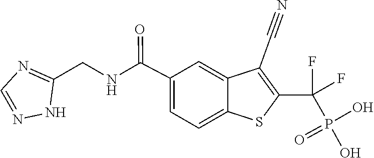 [SMILES: N#Cc1c(C(F)(F)P(=O)(O)O)sc2ccc(C(=O)NCc3ncn[nH]3)cc12] | 414 |
| 172 | 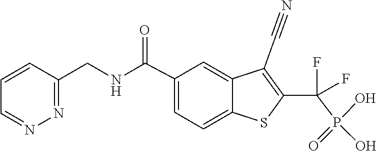 [SMILES: N#Cc1c(C(F)(F)P(=O)(O)O)sc2ccc(C(=O)NCc3cccnn3)cc12] | 425 |
| 173 | 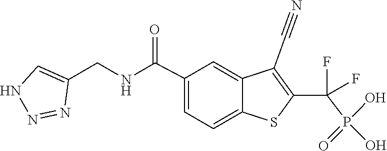 [SMILES: N#Cc1c(C(F)(F)P(=O)(O)O)sc2ccc(C(=O)NCc3c[nH]nn3)cc12] | 414 |
| 174 | 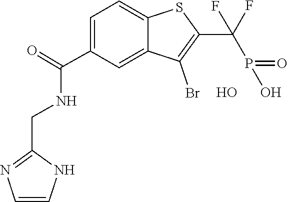 | 466, 468 |
| 175 | 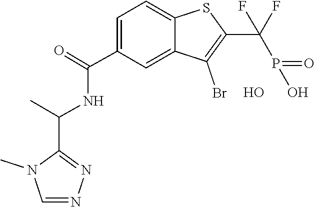 | 495, 497 |
| 176 | 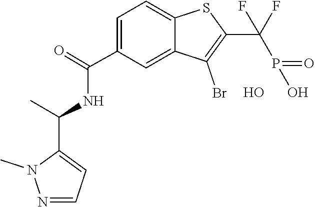 | 494, 496 |
| 177 | 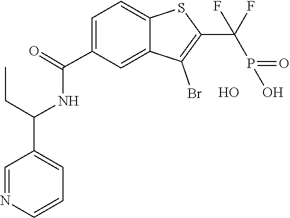 | 505, 507 |
| 178 | 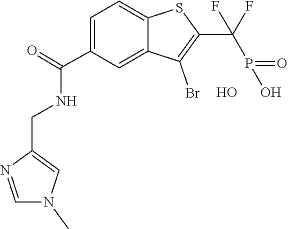 | 480, 482 |
| 179 | 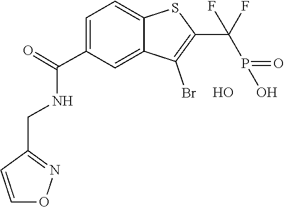 | 467, 469 |
| 180 | 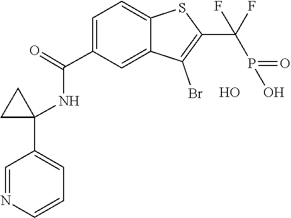 | 503, 505 |
| 181 | 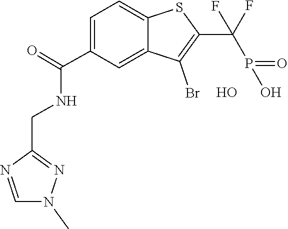 | 481, 483 |
| 182 | 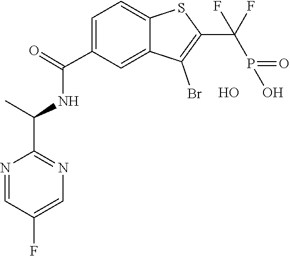 | 510, 512 |
| 183 | 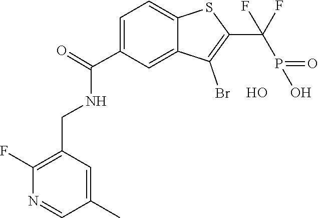 | 509, 511 |
| 184 | 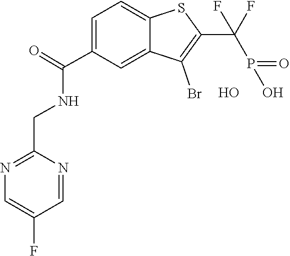 | 496, 498 |
| 185 | 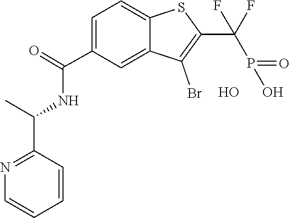 | 491, 493 |
| 186 | 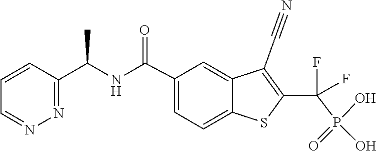 [SMILES: C[C@@H](NC(=O)c1ccc2sc(C(F)(F)P(=O)(O)O)c(C#N)c2c1)c1cccnn1] | 439 |
| 187 | 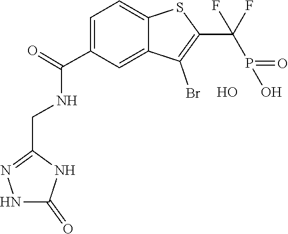 | 483, 485 |
| 188 | 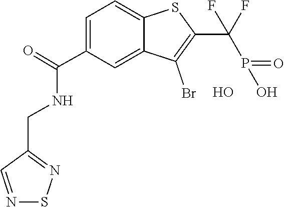 | 484, 486 |
| 189 | 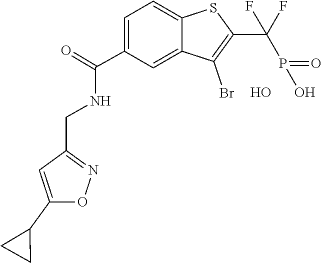 | 507, 509 |
| 190 | 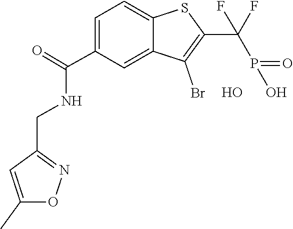 | 481, 483 |
| 191 | 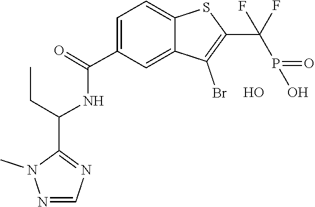 | 509, 511 |
| 192 | 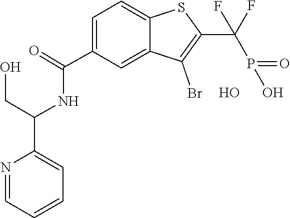 | 507, 509 |
| 193 | 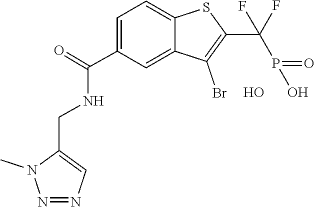 | 481, 483 |
| 194 | 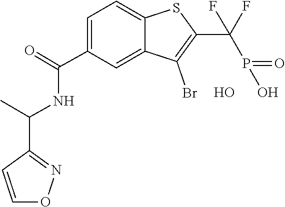 | 481, 483 |
| 195 | 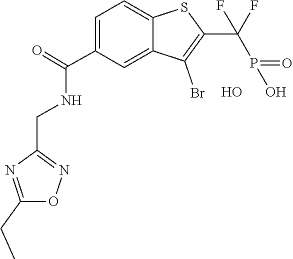 | 496, 498 |
| 196 | 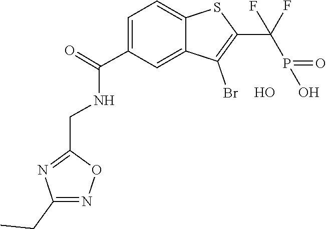 | 496, 498 |
| 197 | 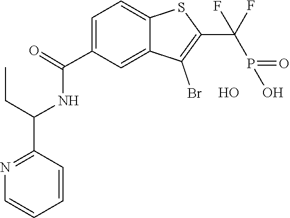 | 505, 507 |
| 198 | 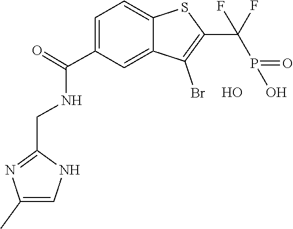 | 480, 482 |
| 199 | 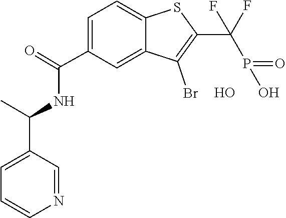 | 491, 493 |
| 200 | 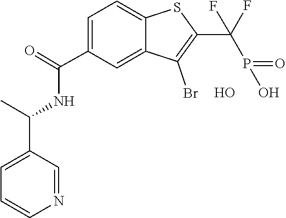 | 491, 493 |
| 201 | 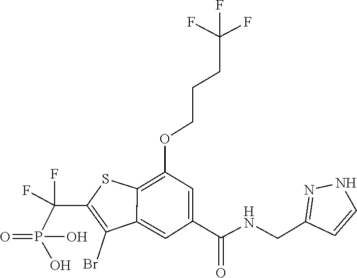 [SMILES: O=C(NCc1cc[nH]n1)c1cc(OCCCC(F)(F)F)c2sc(C(F)(F)P(=O)(O)O)c(Br)c2c1] | 592, 594 |
| 202 | 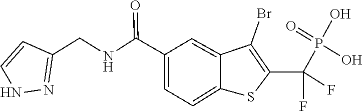 [SMILES: O=C(NCc1cc[nH]n1)c1ccc2sc(C(F)(F)P(=O)(O)O)c(Br)c2c1] | 466, 468 |
| 203 | 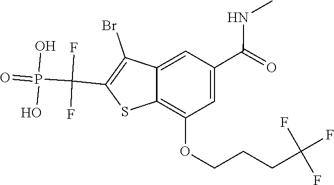 [SMILES: CNC(=O)c1cc(OCCCC(F)(F)F)c2sc(C(F)(F)P(=O)(O)O)c(Br)c2c1] | 526, 528 |
| 204 | 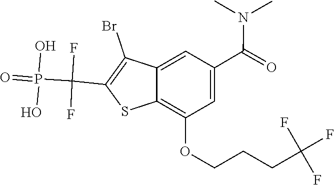 [SMILES: CN(C)C(=O)c1cc(OCCCC(F)(F)F)c2sc(C(F)(F)P(=O)(O)O)c(Br)c2c1] | 540, 542 |
| 205 | 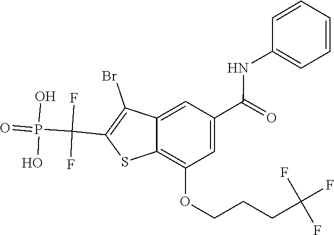 [SMILES: O=C(Nc1ccccc1)c1cc(OCCCC(F)(F)F)c2sc(C(F)(F)P(=O)(O)O)c(Br)c2c1] | 588, 590 |
| 206 | 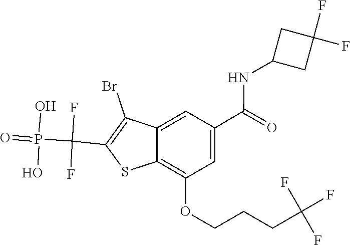 [SMILES: O=C(NC1CC(F)(F)C1)c1cc(OCCCC(F)(F)F)c2sc(C(F)(F)P(=O)(O)O)c(Br)c2c1] | 602, 604 |
| 207 | 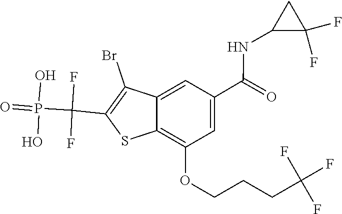 [SMILES: O=C(NC1CC1(F)F)c1cc(OCCCC(F)(F)F)c2sc(C(F)(F)P(=O)(O)O)c(Br)c2c1] | 588, 590 |
| 208 | 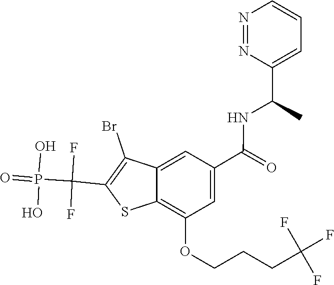 [SMILES: C[C@@H](NC(=O)c1cc(OCCCC(F)(F)F)c2sc(C(F)(F)P(=O)(O)O)c(Br)c2c1)c1cccnn1] | 618, 620 |
| 209 | 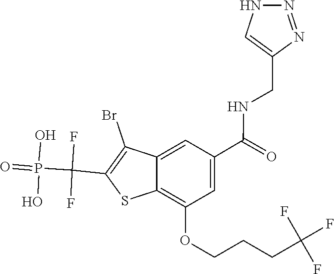 [SMILES: O=C(NCc1c[nH]nn1)c1cc(OCCCC(F)(F)F)c2sc(C(F)(F)P(=O)(O)O)c(Br)c2c1] | 593, 595 |
| 210 | 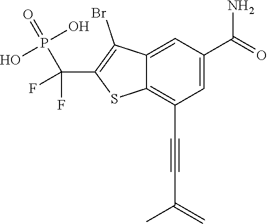 [SMILES: C=C(C)C#Cc1cc(C(N)=O)cc2c(Br)c(C(F)(F)P(=O)(O)O)sc12] | 450, 452 |
| 211 | 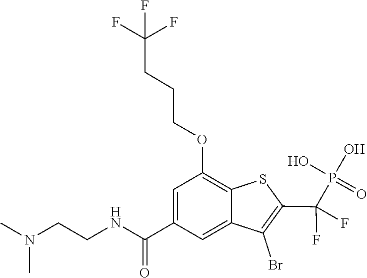 [SMILES: CN(C)CCNC(=O)c1cc(OCCCC(F)(F)F)c2sc(C(F)(F)P(=O)(O)O)c(Br)c2c1] | 583, 585 |
| 304 | 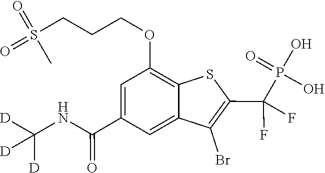 [SMILES: [2H]C([2H])([2H])NC(=O)c1cc(OCCCS(C)(=O)=O)c2sc(C(F)(F)P(=O)(O)O)c(Br)c2c1] | 539, 541 |
| 305 | 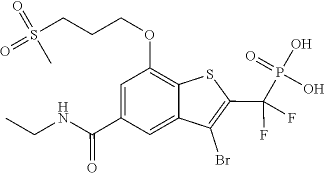 [SMILES: CCNC(=O)c1cc(OCCCS(C)(=O)=O)c2sc(C(F)(F)P(=O)(O)O)c(Br)c2c1] | 550, 552 |
| 306 | 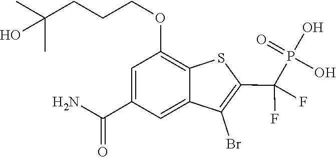 [SMILES: CC(C)(O)CCCOc1cc(C(N)=O)cc2c(Br)c(C(F)(F)P(=O)(O)O)sc12] | 500, 502 [M − H]⁻ |
| 307 | 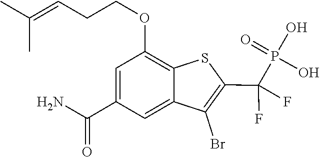 [SMILES: CC(C)=CCCOc1cc(C(N)=O)cc2c(Br)c(C(F)(F)P(=O)(O)O)sc12] | 484, 486 |
| 308 | 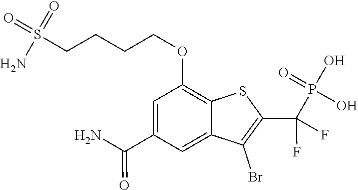 [SMILES: NC(=O)c1cc(OCCCCS(N)(=O)=O)c2sc(C(F)(F)P(=O)(O)O)c(Br)c2c1] | 535, 537 [M − H]⁻ |
| 309 | 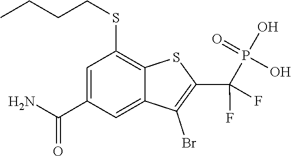 [SMILES: CCCCSc1cc(C(N)=O)cc2c(Br)c(C(F)(F)P(=O)(O)O)sc12] | 474, 476 |
| 310 | 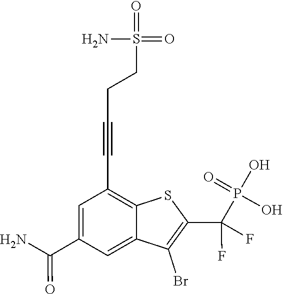 [SMILES: NC(=O)c1cc(C#CCCS(N)(=O)=O)c2sc(C(F)(F)P(=O)(O)O)c(Br)c2c1] | 515, 517 [M − H]⁻ |
| 311 | 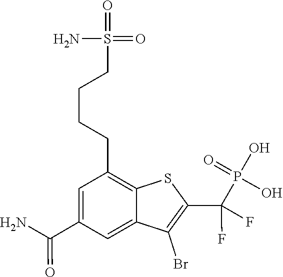 [SMILES: NC(=O)c1cc(CCCCS(N)(=O)=O)c2sc(C(F)(F)P(=O)(O)O)c(Br)c2c1] | 519, 521 [M − H]⁻ |
| 312 | 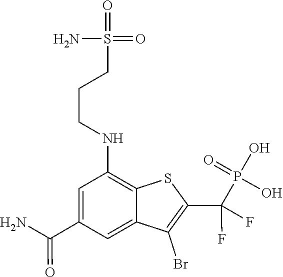 [SMILES: NC(=O)c1cc(NCCCS(N)(=O)=O)c2sc(C(F)(F)P(=O)(O)O)c(Br)c2c1] | 522, 524 |
| 313 | 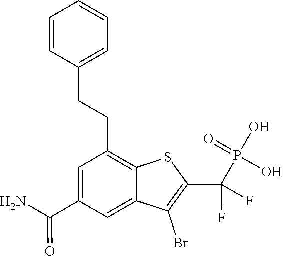 [SMILES: NC(=O)c1cc(CCc2ccccc2)c2sc(C(F)(F)P(=O)(O)O)c(Br)c2c1] | 488, 490 [M − H]⁻ |
| 314 | 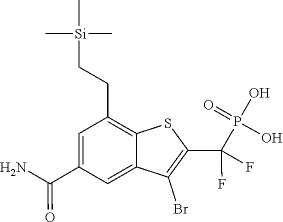 [SMILES: C[Si](C)(C)CCc1cc(C(N)=O)cc2c(Br)c(C(F)(F)P(=O)(O)O)sc12] | 484, 486 [M − H]⁻ |

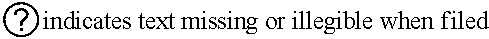

### Example 7

### Preparation of Compound 212

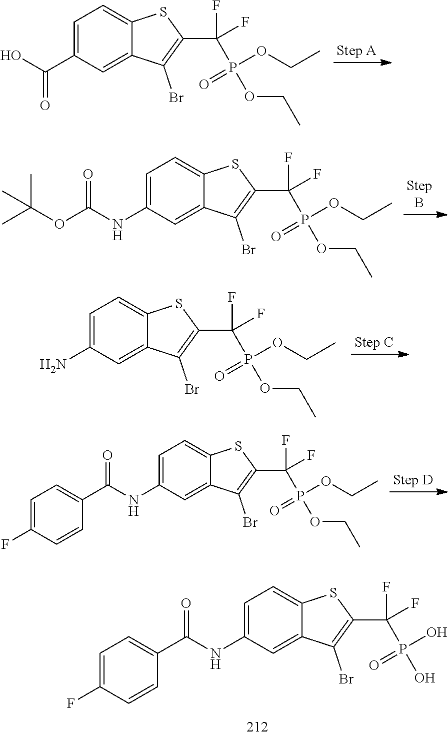

### Step A: Synthesis of tert-butyl (3-bromo-2-((diethoxyphosphoryl)difluoromethyl)benzo[b]thiophen-5-yl)carbamate

A mixture of compound Int-1 (300 mg, 0.677 mmol), diphenylphosphoryl azide (0.18 mL, 0.81 mmol), tert-butanol (0.65 mL, 6.8 mmol), and Hünig's base (0.154 mL, 0.880 mmol) in toluene (3.0 mL) was stirred and heated to 90° C. for 3 hours. The reaction mixture was cooled to room temperature, and directly purified using silica gel chromatography (eluting ethyl acetate in hexanes) to provide tert-butyl (3-bromo-2-((diethoxyphosphoryl)difluoromethyl)benzo[b]thiophen-5-yl)carbamate. MS (ESI, m/z): 536, 538 (M+Na)⁺

### Step B: Synthesis of diethyl ((5-amino-3-bromobenzo[b]thiophen-2-yl)difluoromethyl)phosphonate

TFA (2.4 mL, 31 mmol) was added to a mixture of tert-butyl (3-bromo-2-((diethoxyphosphoryl)difluoromethyl)benzo[b]thiophen-5-yl)carbamate (320 mg, 0.622 mmol) in dichloromethane (4 mL) at room temperature. The mixture was allowed to stir at room temperature for 1 hour. The mixture was concentrated in vacuo and The resulting residue was partitioned between dichloromethane (100 mL), and saturated aqueous sodium bicarbonate (25 mL). The organic layer was separated and washed with additional saturated aqueous sodium bicarbonate (25 mL). The organic layer was separated, dried over magnesium sulfate, filtered, and concentrated in vacuo to provide diethyl ((5-amino-3-bromobenzo[b]thiophen-2-yl)difluoromethyl)phosphonate, which was used without purification in the next step. MS (ESI, m/z): 414, 416 (M+H)⁺

### Step C: Synthesis of diethyl ((3-bromo-5-(4-fluorobenzamido)benzo[b]thiophen-2-yl)difluoromethyl)phosphonate

Triethylamine (0.108 mL, 0.773 mmol) was added to a mixture of diethyl ((5-amino-3-bromobenzo[b]thiophen-2-yl)difluoromethyl)phosphonate (64 mg, 0.16 mmol), and 4-fluorobenzoyl chloride (31 mg, 0.19 mmol) in dichloromethane (2.0 mL) at room temperature. The mixture was allowed to stir for 2 hours at room temperature. The reaction mixture was directly purified using silica gel chromatography (eluting ethyl acetate in dichloromethane) to provide diethyl ((3-bromo-5-(4-fluorobenzamido)benzo[b]thiophen-2-yl)difluoromethyl)phosphonate. MS (ESI, m/z): 536, 538 (M+H)⁺

### Step D: Synthesis of ((3-bromo-5-(4-fluorobenzamido)benzo[b]thiophen-2-yl)difluoromethyl)phosphonic acid

TMS-Br (0.71 mL, 5.5 mmol) was added to a mixture of diethyl ((3-bromo-5-(4-fluorobenzamido)benzo[b]thiophen-2-yl)difluoromethyl)phosphonate (59 mg, 0.11 mmol) in dichloromethane (2.0 mL) at 20° C. The mixture was allowed to stir at room temperature for 18 hours. The mixture was cooled to 0° C. and quenched with methanol (2 mL). The mixture was allowed to stir for 30 minutes at room temperature, and then concentrated in vacuo, and the resulting residue was taken up in methanol (2 mL), filtered, and purified using reverse phase HPLC (eluting acetonitrile in water, with TFA modifier) to provide ((3-bromo-5-(4-fluorobenzamido)benzo[b]thiophen-2-yl)difluoromethyl)phosphonic acid (compound 212). ¹H NMR (499 MHz, DMSO-d₆) δ 10.57 (s, 1H), 8.51 (d, J=1.7 Hz, 1H), 8.13-8.07 (m, 3H), 7.93 (dd, J=8.8, 1.8 Hz, 1H), 7.43-7.38 (m, 2H). MS (ESI, m/z): 480, 482 (M+H)⁺

The following illustrative compounds were made using the methods described in the Example immediately above, and substituting the appropriate reactants and/or reagents.

| | | Mass |
|---|---|---|
| Compound | Structure | [M + H]⁺ |
| 213 | 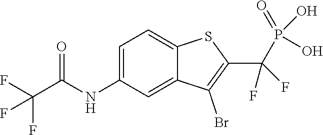 [SMILES: O=C(Nc1ccc2sc(C(F)(F)P(=O)(O)O)c(Br)c2c1)C(F)(F)F] | 454, 456 |
| 214 | 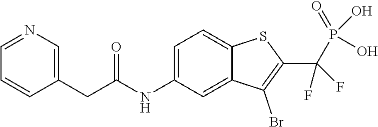 [SMILES: O=C(Cc1cccnc1)Nc1ccc2sc(C(F)(F)P(=O)(O)O)c(Br)c2c1] | 477, 479 |

### Example 8

### Preparation of Compound 215

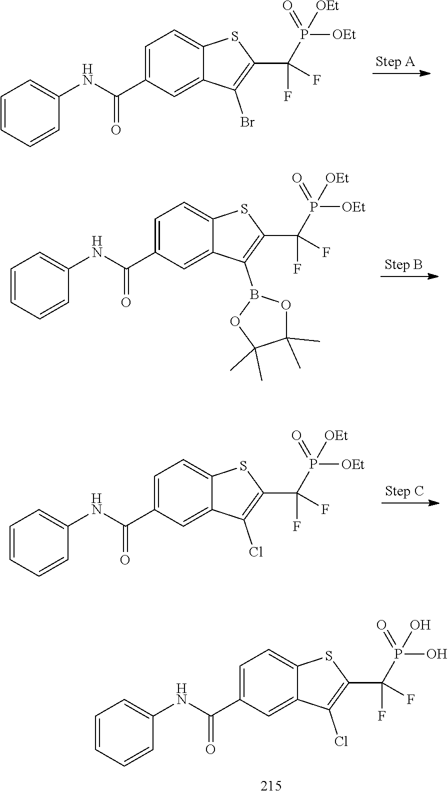 [SMILES: CCOP(=O)(OCC)C(F)(F)c1sc2ccc(C(=O)Nc3ccccc3)cc2c1Br ; CCOP(=O)(OCC)C(F)(F)c1sc2ccc(C(=O)Nc3ccccc3)cc2c1B1OC(C)(C)C(C)(C)O1 ; CCOP(=O)(OCC)C(F)(F)c1sc2ccc(C(=O)Nc3ccccc3)cc2c1Cl ; O=C(Nc1ccccc1)c1ccc2sc(C(F)(F)P(=O)(O)O)c(Cl)c2c1]

### Step A: Synthesis of diethyl (difluoro(5-(phenylcarbamoyl)-3-(4,4,5,5-tetramethyl-1,3,2-dioxaborolan-2-yl)benzo[b]thiophen-2-yl)methyl)phosphonate

To a mixture of diethyl ((3-bromo-5-(phenylcarbamoyl)benzo[b]thiophen-2-yl)difluoromethyl)phosphonate (50 mg, 0.096 mmol), Pd(OAc)₂ (0.54 mg, 0.0024 mmol), and CyJohnPhos (3.4 mg, 0.0097 mmol) in 1,4-dioxane (0.5 mL) was added TEA (0.054 mL, 0.39 mmol), and 4,4,5,5-tetramethyl-1,3,2-dioxaborolane (0.035 mL, 0.24 mmol) at room temperature under argon. The resulting mixture was allowed to stir for 2 hours at 80° C. The reaction was cooled to room temperature, diluted with water (10 mL), and extracted with EtOAc (2×20 mL). The combined organic extracts were dried over anhydrous Na₂SO₄ and concentrated in vacuo to provide diethyl (difluoro(5-(phenylcarbamoyl)-3-(4,4,5,5-tetramethyl-1,3,2-dioxaborolan-2-yl)benzo[b]thiophen-2-yl)methyl)phosphonate, which was used without purification in the next step. MS (ESI, m/z): 566 (M+H)⁺

### Step B: Synthesis of diethyl ((3-chloro-5-(phenylcarbamoyl)benzo[b]thiophen-2-yl)difluoromethyl)phosphonate

A mixture of diethyl (difluoro(5-(phenylcarbamoyl)-3-(4,4,5,5-tetramethyl-1,3,2-dioxaborolan-2-yl)benzo[b]thiophen-2-yl)methyl)phosphonate (25 mg, 0.044 mmol), and CuCl₂ (5.9 mg, 0.044 mmol) in water (0.75 mL), and MeOH (0.75 mL) was allowed to stir for 16 hours at 65° C. The reaction was cooled to room temperature, diluted with water (4 mL), and extracted with EtOAc (2×10 mL). The combined organic extracts were dried over anhydrous Na₂SO₄ and concentrated in vacuo. The resulting residue was purified using reverse phase HPLC (eluting acetonitrile in water with NH₄OAc modifier) to provide diethyl ((3-chloro-5-(phenylcarbamoyl)benzo[b]thiophen-2-yl)difluoromethyl)phosphonate. MS (ESI, m/z): 472 (M−H)⁻

### Step C: Synthesis of ((3-chloro-5-(phenylcarbamoyl)benzo[b]thiophen-2-yl)difluoromethyl)phosphonic acid

To a solution of diethyl ((3-chloro-5-(phenylcarbamoyl)benzo[b]thiophen-2-yl)difluoromethyl)phosphonate (7.0 mg, 0.015 mmol) in DMF (0.1 mL) was added TMSBr (0.058 mL, 0.44 mmol) under argon. The resulting solution was allowed to stir for 1.5 hours at 60° C. The reaction was cooled to room temperature, and concentrated in vacuo. The resulting residue was co-evaporated with DCM (2×2 mL), and EtOH (2×2 mL). The obtained residue was purified using reverse phase HPLC (eluting acetonitrile in water with NH₄HCO₃ modifier) to provide ((3-chloro-5-(phenylcarbamoyl)benzo[b]thiophen-2-yl)difluoromethyl)phosphonic acid (compound 215). ¹H NMR (400 MHz, D₂O) δ 8.47-8.37 (m, 1H), 8.15-8.03 (m, 1H), 8.01-7.91 (m, 1H), 7.66-7.55 (m, 2H), 7.53-7.44 (m, 2H), 7.37-7.26 (m, 1H). MS (ESI, m/z): 418 (M+H)⁺

### Example 9

### Preparation of Compound 216

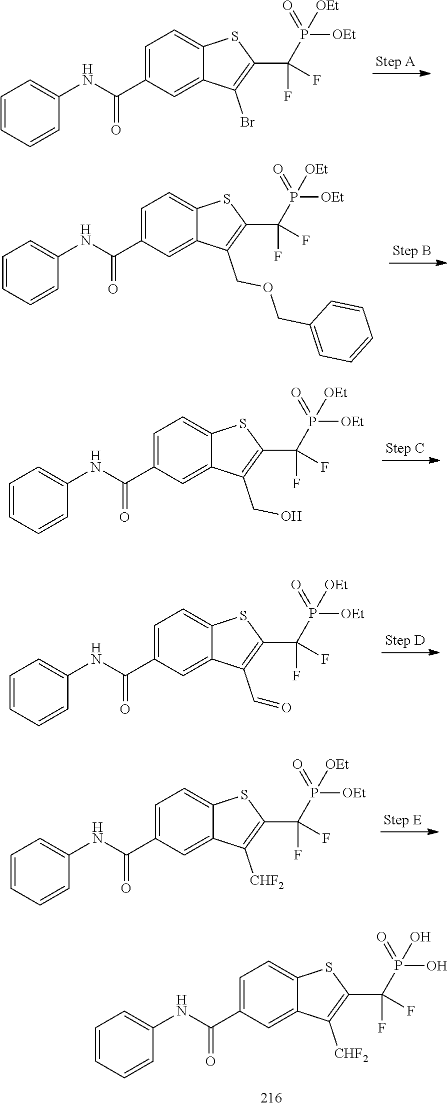

### Step A: Synthesis of diethyl ((3-((benzyloxy)methyl)-5-(phenylcarbamoyl)benzo[b]thiophen-2-yl)difluoromethyl)phosphonate

To a mixture of diethyl ((3-bromo-5-(phenylcarbamoyl)benzo[b]thiophen-2-yl)difluoromethyl)phosphonate (330 mg, 0.64 mmol), potassium ((benzyloxy)methyl)trifluoroborate (170 mg, 0.76 mmol), and cesium carbonate (750 mg, 2.3 mmol) in toluene (3 mL) was added chloro(2-dicyclohexylphosphino-2′,6′-diisopropyl-1,1′biphenyl)[2-(2′-amino-1,1′-biphenyl)]palladium(II) (47 mg, 0.064 mmol) at room temperature under argon. The resulting mixture was allowed to stir for 16 hours at 80° C., then the reaction mixture was cooled to room temperature, filtered, and concentrated in vacuo. The resulting residue was purified using reverse phase HPLC (eluting acetonitrile in water) to provide diethyl ((3-((benzyloxy)methyl)-5-(phenylcarbamoyl)benzo[b]thiophen-2-yl)difluoromethyl)phosphonate. MS (ESI, m/z): 558 (M−H)⁻

### Step B: Synthesis of diethyl (difluoro(3-(hydroxymethyl)-5-(phenylcarbamoyl)benzo[b]thiophen-2-yl)methyl)phosphonate

To a mixture of diethyl ((3-((benzyloxy)methyl)-5-(phenylcarbamoyl)benzo[b]thiophen-2-yl)difluoromethyl)phosphonate (78 mg, 0.14 mmol) in DCM (1 mL) was added a solution of boron trichloride in DCM (1M) (0.63 mL, 0.63 mmol) at −78° C. under argon. The reaction was held at −78° C. for 3 hours then quenched with MeOH (0.5 mL). The resulting solution was concentrated in vacuo and purified using reverse phase HPLC (eluting acetonitrile in water) to provide diethyl (difluoro(3-(hydroxymethyl)-5-(phenylcarbamoyl)benzo[b]thiophen-2-yl)methyl)phosphonate. MS (ESI, m/z): 468 (M−H)⁻

### Step C: Synthesis of diethyl (difluoro(3-formyl-5-(phenylcarbamoyl)benzo[b]thiophen-2-yl)methyl)phosphonate

To a solution of diethyl (difluoro(3-(hydroxymethyl)-5-(phenylcarbamoyl)benzo[b]thiophen-2-yl)methyl)phosphonate (30 mg, 0.064 mmol) in DCM (0.6 mL) was added a Dess-Martin periodinane (27 mg, 0.064 mmol) at 0° C. under argon. The reaction was warmed to room temperature, and stirred for 2 hours. The reaction was directly purified using reverse phase HPLC (eluting acetonitrile in water) to provide diethyl (difluoro(3-formyl-5-(phenylcarbamoyl)benzo[b]thiophen-2-yl)methyl)phosphonate. MS (ESI, m/z): 468 (M+H)⁺

### Step D: Synthesis of diethyl ((3-(difluoromethyl)-5-(phenylcarbamoyl)benzo[b]thiophen-2-yl)difluoromethyl)phosphonate

To a solution of diethyl (difluoro(3-formyl-5-(phenylcarbamoyl)benzo[b]thiophen-2-yl)methyl)phosphonate (16 mg, 0.034 mmol) in DCM (0.2 mL) was added DAST (0.014 mL, 0.1 mmol) at 0° C. under argon. The reaction was warmed to room temperature, and stirred for 2 hours. The reaction was directly purified using reverse phase HPLC (eluting acetonitrile in water with NH₄HCO₃ modifier) to provide diethyl ((3-(difluoromethyl)-5-(phenylcarbamoyl)benzo[b]thiophen-2-yl)difluoromethyl)phosphonate. MS (ESI, m/z): 490 (M+H)⁺

### Step E: Synthesis of ((3-(difluoromethyl)-5-(phenylcarbamoyl)benzo[b]thiophen-2-yl)difluoromethyl)phosphonic acid

To a solution of diethyl ((3-(difluoromethyl)-5-(phenylcarbamoyl)benzo[b]thiophen-2-yl)difluoromethyl)phosphonate (5.0 mg, 0.01 mmol) in DMF (0.2 mL) was added TMS-Br (0.04 mL, 0.31 mmol) under argon. The resulting solution was allowed to stir for 1.5 hours at 60° C. The reaction was cooled to room temperature, quenched by addition of EtOH (0.5 mL), and concentrated in vacuo. The resulting residue was purified using reverse phase HPLC (eluting acetonitrile in water with NH₄HCO₃ modifier) to provide ((3-(difluoromethyl)-5-(phenylcarbamoyl)benzo[b]thiophen-2-yl)difluoromethyl)phosphonic acid (compound 216). ¹H NMR (300 MHz, D₂O) δ 8.55 (s, 1H), 8.04 (d, J=8.6 Hz, 1H), 7.84 (d, J=8.9 Hz, 1H), 7.56-7.12 (m, 6H). MS (ESI, m/z): 432 (M−H)⁻

### Example 10

### Preparation of Compound 217

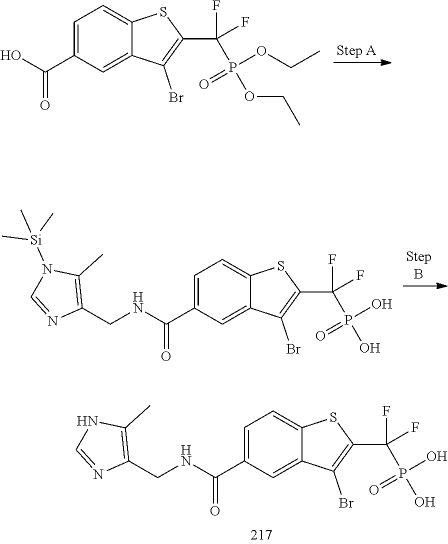

### Step A: Synthesis of ((3-bromo-5-(((5-methyl-1-(trimethylsilyl)-1H-imidazol-4-yl)methyl)carbamoyl)benzo[b]thiophen-2-yl)difluoromethyl)phosphonic acid

A mixture of compound Int-1 (25 mg, 0.056 mmol), (5-methyl-1H-imidazol-4-yl)methanamine (12 mg, 0.11 mmol), HATU (43 mg, 0.11 mmol), and Hünig's base (0.029 mL, 0.17 mmol) in DMF (0.5 mL) was allowed to stir at room temperature for 30 minutes. TMS-Br (0.22 mL, 1.7 mmol) was added and the mixture was allowed to stir at room temperature for 18 hours. The reaction was quenched with methanol, then concentrated in vacuo to provide ((3-bromo-5-(((5-methyl-1-(trimethylsilyl)-1H-imidazol-4-yl)methyl)carbamoyl)benzo[b]thiophen-2-yl)difluoromethyl)phosphonic acid, which was used without purification in the next step.

### Step B: Synthesis of ((3-bromo-5-(((5-methyl-1H-imidazol-4-yl)methyl)carbamoyl)benzo[b]thiophen-2-yl)difluoromethyl)phosphonic acid

A solution of TBAF (1.0 M in THF, 330 μl, 0.330 mmol) was added directly to ((3-bromo-5-(((5-methyl-1-(trimethylsilyl)-1H-imidazol-4-yl)methyl)carbamoyl)benzo[b]thiophen-2-yl)difluoromethyl)phosphonic acid (31 mg, 0.056 mmol). The mixture was stirred and heated to 50° C. for 30 minutes, then the reaction mixture was concentrated in vacuo and The resulting residue was purified using reverse phase HPLC (eluting acetonitrile in water, with TFA modifier) to provide ((3-bromo-5-(((5-methyl-1H-imidazol-4-yl)methyl)carbamoyl)benzo[b]thiophen-2-yl)difluoromethyl)phosphonic acid (compound 217).

¹H NMR (600 MHz, DMSO-d₆) δ 9.93 (br s, 1H), 8.88 (s, 1H), 7.94-7.90 (m, 2H), 7.73 (d, J=8.4 Hz, 1H), 4.33 (d, J=5.2 Hz, 2H), 2.35 (s, 3H). MS (ESI, m/z): 480, 482 (M+H)⁺

The following illustrative compound was made using the methods described in the Example immediately above, and substituting the appropriate reactants and/or reagents.

| | | Mass |
|---|---|---|
| Compound | Structure | [M + H]⁺ |
| 218 | 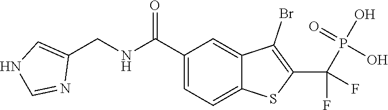 [SMILES: O=C(NCc1c[nH]cn1)c1ccc2sc(C(F)(F)P(=O)(O)O)c(Br)c2c1] | 466, 468 |

### Example 11

### Preparation of Compound 219

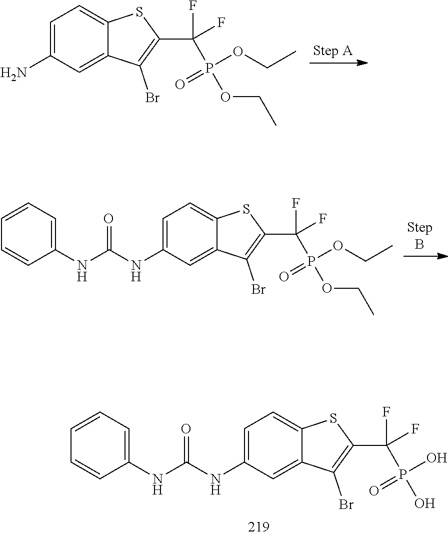 [SMILES: CCOP(=O)(OCC)C(F)(F)c1sc2ccc(N)cc2c1Br ; CCOP(=O)(OCC)C(F)(F)c1sc2ccc(NC(=O)Nc3ccccc3)cc2c1Br ; O=C(Nc1ccccc1)Nc1ccc2sc(C(F)(F)P(=O)(O)O)c(Br)c2c1]

### Step A: Synthesis of diethyl ((3-bromo-5-(3-phenylureido)benzo[b]thiophen-2-yl)difluoromethyl)phosphonate

Isocyanatobenzene (41.4 mg, 0.348 mmol) was added to a mixture of diethyl ((5-amino-3-bromobenzo[b]thiophen-2-yl)difluoromethyl)phosphonate (72 mg, 0.17 mmol) in DCM (2.0 mL) at room temperature. The reaction was allowed to stir at room temperature for 1 hour, then the reaction mixture was directly purified using silica gel chromatography (eluting ethyl acetate in dichloromethane) to provide diethyl ((3-bromo-5-(3-phenylureido)benzo[b]thiophen-2-yl)difluoromethyl)phosphonate. MS (ESI, m/z): 533, 535 (M+H)⁺

### Step B: Synthesis of ((3-bromo-5-(3-phenylureido)benzo[b]thiophen-2-yl)difluoromethyl)phosphonic acid

TMS-Br (0.41 mL, 3.2 mmol) was added to a mixture of diethyl ((3-bromo-5-(3-phenylureido)benzo[b]thiophen-2-yl)difluoromethyl)phosphonate (68 mg, 0.13 mmol) in dichloromethane (2.0 mL) at room temperature. The resulting reaction was allowed to stir at room temperature for 18 hours, then the reaction mixture was cooled to 0° C., and quenched with methanol (2 mL). The mixture was allowed to stir for 30 minutes at room temperature, and then concentrated in vacuo, and the resulting residue was taken up in methanol (2 mL), filtered, and purified using reverse phase HPLC (eluting acetonitrile in water, with 0.1% TFA modifier) to provide ((3-bromo-5-(3-phenylureido)benzo[b]thiophen-2-yl)difluoromethyl)phosphonic acid (compound 219). ¹H NMR (499 MHz, DMSO-d₆) δ 9.06 (s, 1H), 8.76 (s, 1H), 8.26 (s, 1H), 7.99 (d, J=8.7 Hz, 1H), 7.51-7.45 (m, 3H), 7.30 (t, J=7.8 Hz, 2H), 6.99 (t, J=7.3 Hz, 1H). MS (ESI, m/z): 477, 479 (M+H)⁺

### Example 12

### Preparation of Compound 220

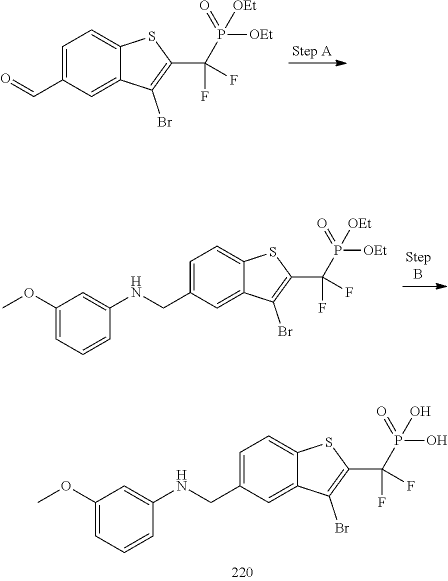 [SMILES: CCOP(=O)(OCC)C(F)(F)c1sc2ccc(C=O)cc2c1Br ; CCOP(=O)(OCC)C(F)(F)c1sc2ccc(CNc3cccc(OC)c3)cc2c1Br ; COc1cccc(NCc2ccc3sc(C(F)(F)P(=O)(O)O)c(Br)c3c2)c1]

### Step A: Synthesis of diethyl ((3-bromo-5-(((3-methoxyphenyl)amino)methyl)benzo[b]thiophen-2-yl)difluoromethyl)phosphonate

To a mixture of diethyl ((3-bromo-5-formylbenzo[b]thiophen-2-yl)difluoromethyl)phosphonate (50 mg, 0.12 mmol) in MeOH (0.5 mL) was added 3-methoxyaniline (22 mg, 0.18 mmol), and sodium borohydride (8.9 mg, 0.23 mmol) at 0° C. under argon. The resulting mixture was warmed to room temperature, and stirred for 2 hours. The reaction was quenched with water (0.3 mL), and directly purified using reverse phase HPLC (eluting acetonitrile in water with NH₄HCO₃ modifier) to provide diethyl ((3-bromo-5-(((3-methoxyphenyl)amino)methyl)benzo[b]thiophen-2-yl)difluoromethyl)phosphonate. ¹H NMR (300 MHz, CDCl₃) δ 7.92 (s, 1H), 7.79 (d, J=8.3 Hz, 1H), 7.52 (d, J=8.7 Hz, 1H), 7.09 (t, J=8.1 Hz, 1H), 6.38-6.27 (m, 2H), 6.25 (d, J=2.3 Hz, 1H), 4.49 (s, 2H), 4.42-4.18 (m, 4H), 3.75 (s, 3H), 1.43-1.32 (m, 6H).

### Step B: Synthesis of ((3-bromo-5-(((3-methoxyphenyl)amino)methyl)benzo[b]thiophen-2-yl)difluoromethyl)phosphonic acid

To a solution of diethyl ((3-bromo-5-(((3-methoxyphenyl)amino)methyl)benzo[b]thiophen-2-yl)difluoromethyl)phosphonate (15 mg, 0.028 mmol) in DMF (0.2 mL) was added TMS-Br (0.06 mL, 0.46 mmol) under argon. The resulting solution was allowed to stir for 1 hour at 60° C. The reaction was cooled to room temperature, quenched by addition of EtOH (0.5 mL), and concentrated in vacuo. The resulting residue was purified using reverse phase HPLC (eluting acetonitrile in water with NH₄HCO₃ modifier) to ((3-bromo-5-(((3-methoxyphenyl)amino)methyl)benzo[b]thiophen-2-yl)difluoromethyl)phosphonic acid (compound 220). ¹H NMR (400 MHz, D₂O) δ 7.94-7.86 (m, 2H), 7.49 (d, J=8.3 Hz, 1H), 7.11 (t, J=8.2 Hz, 1H), 6.46 (d, J=8.1 Hz, 1H), 6.42-6.33 (m, 2H), 4.50 (s, 2H), 3.72 (s, 3H). MS (ESI, m/z): 478, 480 (M+H)⁺

### Example 13

### Preparation of Compound 221

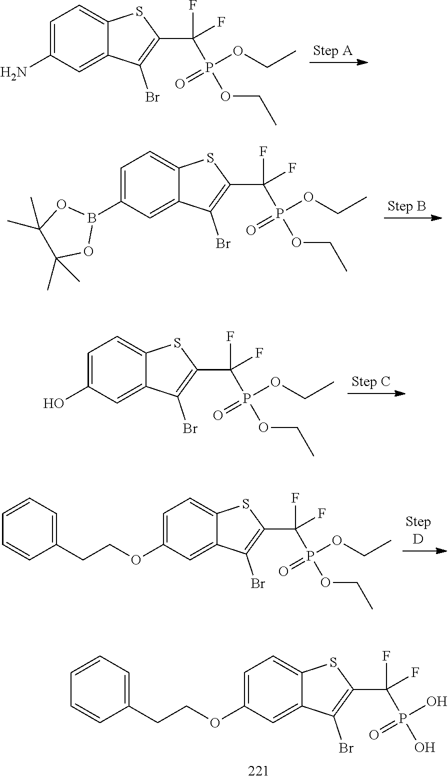

### Step A: Synthesis of diethyl ((3-bromo-5-(4,4,5,5-tetramethyl-1,3,2-dioxaborolan-2-yl)benzo[b]thiophen-2-yl)difluoromethyl)phosphonate

A solution of sodium nitrite (48 mg, 0.70 mmol) in water (500 μL) was added dropwise to a mixture of diethyl ((5-amino-3-bromobenzo[b]thiophen-2-yl)difluoromethyl)phosphonate (262 mg, 0.633 mmol), and hydrochloric acid (1.0 M in water, 1.26 mL, 1.26 mmol) in methanol (5.0 mL) at 0° C. The mixture was allowed to stir at 0° C. for 15 minutes. Bis(pinacolato)diboron (643 mg, 2.53 mmol) was added to the mixture at 0° C. and the mixture was warmed to room temperature, and stirred for 4 hours. The mixture was diluted with water (10 mL), and extracted with DCM (100 mL). The organic layer was separated, washed with brine (10 mL), dried over magnesium sulfate, filtered, and concentrated in vacuo to provide diethyl ((3-bromo-5-(4,4,5,5-tetramethyl-1,3,2-dioxaborolan-2-yl)benzo[b]thiophen-2-yl)difluoromethyl)phosphonate, which was used in the next step without purification. MS (ESI, m/z): 525, 527 (M+H)⁺

### Step B: Synthesis of diethyl ((3-bromo-5-hydroxybenzo[b]thiophen-2-yl)difluoromethyl)phosphonate

A solution of Oxone (0.20 M in water, 13 mL, 2.5 mmol) was added to a mixture of diethyl ((3-bromo-5-(4,4,5,5-tetramethyl-1,3,2-dioxaborolan-2-yl)benzo[b]thiophen-2-yl)difluoromethyl)phosphonate (664 mg, 1.26 mmol) in acetone (30 mL) at room temperature. The mixture was allowed to stir for 1 hour at room temperature, diluted with water (40 mL), and extracted with DCM (200 mL). The organic layer was separated, washed with brine (20 mL), dried over magnesium sulfate, filtered, and concentrated in vacuo, and the resulting residue was purified using silica gel chromatography (eluting ethyl acetate in dichloromethane) to provide diethyl ((3-bromo-5-hydroxybenzo[b]thiophen-2-yl)difluoromethyl)phosphonate. MS (ESI, m/z): 415, 417 (M+H)⁺

### Step C: Synthesis of diethyl ((3-bromo-5-phenethoxybenzo[b]thiophen-2-yl)difluoromethyl)phosphonate

A mixture of DIAD (0.056 mL, 0.29 mmol) in tetrahydrofuran (0.75 mL) was added to a mixture of diethyl ((3-bromo-5-hydroxybenzo[b]thiophen-2-yl)difluoromethyl)phosphonate (80 mg, 0.19 mmol), 2-phenylethan-1-ol (35 mg, 0.29 mmol), and triphenylphosphine (76 mg, 0.29 mmol) in tetrahydrofuran (0.50 mL) at room temperature. The mixture was allowed to stir at room temperature for 2 hours. The reaction mixture was directly purified using silica gel chromatography (eluting ethyl acetate in hexanes) to provide diethyl ((3-bromo-5-phenethoxybenzo[b]thiophen-2-yl)difluoromethyl)phosphonate. MS (ESI, m/z): 519, 521 (M+H)⁺

### Step D: Synthesis of ((3-bromo-5-phenethoxybenzo[b]thiophen-2-yl)difluoromethyl)phosphonic acid

TMS-Br (0.44 mL, 3.4 mmol) was added to a mixture of diethyl ((3-bromo-5-phenethoxybenzo[b]thiophen-2-yl)difluoromethyl)phosphonate (88 mg, 0.17 mmol) in dichloromethane (2.0 mL) at room temperature. The mixture was allowed to stir at room temperature for 24 hours. The reaction mixture was cooled to 0° C. and quenched with methanol (2 mL). The mixture was allowed to stir for 30 minutes at room temperature, and then concentrated in vacuo, and the resulting residue was taken up in methanol (2 mL), filtered, and purified using reverse phase HPLC (eluting acetonitrile in water, with 0.1% TFA modifier) to provide ((3-bromo-5-phenethoxybenzo[b]thiophen-2-yl)difluoromethyl)phosphonic acid (compound 221). ¹H NMR (499 MHz, DMSO-d₆) δ 8.00 (d, J=8.9 Hz, 1H), 7.38-7.30 (m, 4H), 7.28-7.17 (m, 3H), 4.32 (t, J=6.7 Hz, 2H), 3.10 (t, J=6.6 Hz, 2H). MS (ESI, m/z): 463, 465 (M+H)⁺

### Example 14

### Preparation of Compound 222a and 222b

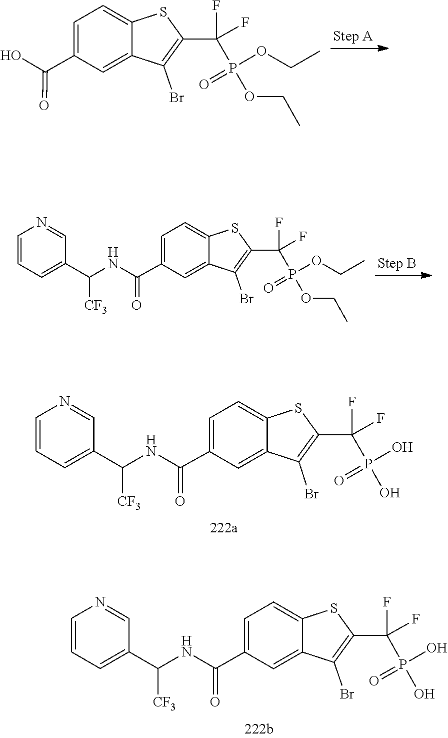 [SMILES: CCOP(=O)(OCC)C(F)(F)c1sc2ccc(C(=O)O)cc2c1Br ; CCOP(=O)(OCC)C(F)(F)c1sc2ccc(C(=O)NC(c3cccnc3)C(F)(F)F)cc2c1Br ; O=C(NC(c1cccnc1)C(F)(F)F)c1ccc2sc(C(F)(F)P(=O)(O)O)c(Br)c2c1 ; O=C(NC(c1cccnc1)C(F)(F)F)c1ccc2sc(C(F)(F)P(=O)(O)O)c(Br)c2c1]

### Step A: Synthesis of (R or S)-diethyl ((3-bromo-5-((2,2,2-trifluoro-1-(pyridin-3-yl)ethyl)carbamoyl)benzo[b]thiophen-2-yl)difluoromethyl)phosphonate and (S or R)-diethyl ((3-bromo-5-((2,2,2-trifluoro-1-(pyridin-3-yl)ethyl)carbamoyl)benzo[b]thiophen-2-yl)difluoromethyl)phosphonate

HATU (343 mg, 0.903 mmol) was added to a mixture of compound Int-1 (200 mg, 0.451 mmol) in DMF (4.5 mL). Hünig's base (292 mg, 2.256 mmol) was added to the mixture (292 mg, 2.26 mmol). The mixture was allowed to stir at room temperature for 5 minutes. (rac)-2,2,2-trifluoro-1-(pyridin-3-yl)ethan-1-amine hydrochloride (192 mg, 0.903 mmol) was then added to the mixture. The reaction mixture was allowed to stir at room temperature for 30 minutes. The mixture was filtered, and directly purified using reverse phase HPLC (eluting acetonitrile/water gradient with 0.1% TFA modifier) to provide the product as a mixture of isomers. The mixture of isomers was resolved by chiral SFC (Chiralpak IA column, 21×250 mm, 5 μm; eluting 25% methanol (with 0.1% ammonium hydroxide modifier) in CO₂) to provide:

Peak 1: (R or S)-diethyl ((3-bromo-5-((2,2,2-trifluoro-1-(pyridin-3-yl)ethyl)carbamoyl)benzo[b]thiophen-2-yl)difluoromethyl)phosphonate (tᵣ=2.20 minutes). MS (ESI, m/z): 601, 603 (M+H)⁺

Peak 2: (S or R)-diethyl ((3-bromo-5-((2,2,2-trifluoro-1-(pyridin-3-yl)ethyl)carbamoyl)benzo[b]thiophen-2-yl)difluoromethyl)phosphonate (tᵣ=3.00 minutes). MS (ESI, m/z): 601, 603 (M+H)⁺

### Step B: Synthesis of (R or S)-((3-bromo-5-((2,2,2-trifluoro-1-(pyridin-3-yl)ethyl)carbamoyl)benzo[b]thiophen-2-yl)difluoromethyl)phosphonic acid and (S or R)-((3-bromo-5-((2,2,2-trifluoro-1-(pyridin-3-yl)ethyl)carbamoyl)benzo[b]thiophen-2-yl)difluoromethyl)phosphonic acid

TMS-Br (518 μl, 3.99 mmol) was added to a mixture of (S or R)-diethyl-((3-bromo-5-((2,2,2-trifluoro-1-(pyridin-3-yl)ethyl)carbamoyl)benzo[b]thiophen-2-yl)difluoromethyl)phosphonate (Peak 1 from previous step) (80 mg, 0.13 mmol) in DCM (1.3 mL). The mixture was allowed to stir at room temperature for 2 days. The mixture was diluted with MeOH (1 mL), filtered, and directly purified using reverse phase HPLC (eluting acetonitrile/water with 0.1% TFA modifier) to provide (S or R)-((3-bromo-5-((2,2,2-trifluoro-1-(pyridin-3-yl)ethyl)carbamoyl)benzo[b]thiophen-2-yl)difluoromethyl)phosphonic acid. ¹H NMR (499 MHz, DMSO-d₆) δ 9.90 (d, J=9.4 Hz, 1H), 8.97 (s, 1H), 8.69 (d, J=4.7 Hz, 1H), 8.41 (s, 1H), 8.28 (d, J=8.4 Hz, 2H), 8.07 (d, J=8.5 Hz, 1H), 7.60 (dd, J=7.8, 5.0 Hz, 1H), 6.32 (p, J=8.8 Hz, 1H). MS (ESI, m/z): 545, 547 (M+H)⁺

In a separate reaction, TMS-Br (518 μl, 3.99 mmol) was added to a mixture of (R or S)-diethyl-((3-bromo-5-((2,2,2-trifluoro-1-(pyridin-3-yl)ethyl)carbamoyl)benzo[b]thiophen-2-yl)difluoromethyl)phosphonate (Peak 2 from previous step) (80 mg, 0.13 mmol) in DCM (1.3 mL). The mixture was allowed to stir at room temperature for 2 days. The mixture was diluted with MeOH (1 mL), filtered, and directly purified using reverse phase HPLC (eluting acetonitrile/water with 0.1% TFA modifier) to provide (R or S)-((3-bromo-5-((2,2,2-trifluoro-1-(pyridin-3-yl)ethyl)carbamoyl)benzo[b]thiophen-2-yl)difluoromethyl)phosphonic acid (compounds 222a and 222b, absolute stereochemistry not determined). ¹H NMR (499 MHz, DMSO-d₆) δ 9.89 (d, J=9.4 Hz, 1H), 8.97 (s, 1H), 8.69 (d, J=4.7 Hz, 1H), 8.41 (s, 1H), 8.30-8.25 (m, 2H), 8.07 (d, J=8.5 Hz, 1H), 7.59 (dd, J=7.9, 4.9 Hz, 1H), 6.32 (p, J=8.5 Hz, 1H). MS (ESI, m/z): 545, 547 (M+H)⁺

### Example 15

### Preparation of Compound 223

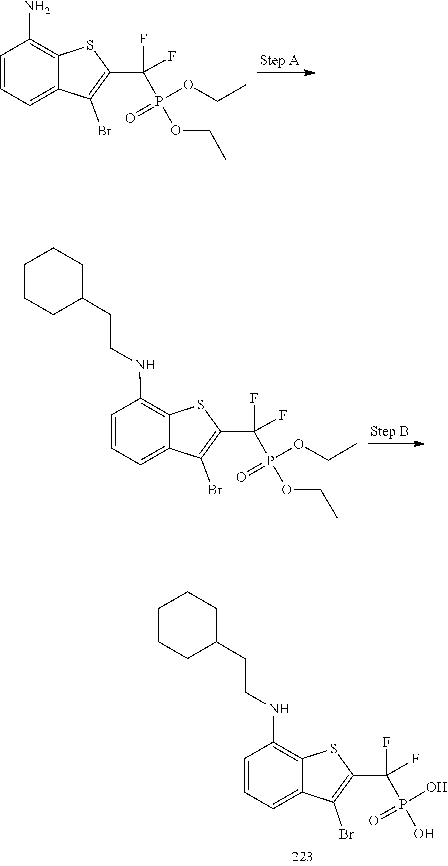 [SMILES: CCOP(=O)(OCC)C(F)(F)c1sc2c(N)cccc2c1Br ; CCOP(=O)(OCC)C(F)(F)c1sc2c(NCCC3CCCCC3)cccc2c1Br ; O=P(O)(O)C(F)(F)c1sc2c(NCCC3CCCCC3)cccc2c1Br]

### Step A: Synthesis of diethyl ((3-bromo-7-((2-cyclohexylethyl)amino)benzo[b]thiophen-2-yl)difluoromethyl)phosphonate

Sodium triacetoxyborohydride (38 mg, 0.18 mmol) was added to a mixture of Int-10 (25 mg, 0.060 mmol), and 2-cyclohexylacetaldehyde (23 mg, 0.18 mmol) in DCE (1.0 mL) at room temperature. The mixture was allowed to stir at room temperature for 6 hours. The reaction mixture was directly purified using silica gel chromatography (eluting [25% ethanol in ethyl acetate]in dichloromethane) to provide diethyl ((3-bromo-7-((2-cyclohexylethyl)amino)benzo[b]thiophen-2-yl)difluoromethyl)phosphonate. MS (ESI, m/z): 524, 526 (M+H)⁺

### Step B: Synthesis of ((3-bromo-7-((2-cyclohexylethyl)amino)benzo[b]thiophen-2-yl)difluoromethyl)phosphonic acid

TMS-Br (0.199 mL, 1.54 mmol) was added to a mixture of diethyl ((3-bromo-7-((2-cyclohexylethyl)amino)benzo[b]thiophen-2-yl)difluoromethyl)phosphonate (23 mg, 0.044 mmol) in dichloromethane (2.0 mL) at room temperature. The mixture was allowed to stir at room temperature for 18 hours. The mixture was cooled to 0° C. and quenched with methanol (2 mL). The mixture was allowed to stir for 30 minutes at room temperature, and then concentrated in vacuo, and the resulting residue was taken up in methanol (2 mL), filtered, and purified using reverse phase HPLC (eluting acetonitrile in water, with 0.1% TFA modifier) to provide ((3-bromo-7-((2-cyclohexylethyl)amino)benzo[b]thiophen-2-yl)difluoromethyl)phosphonic acid (compound 223). ¹H NMR (499 MHz, DMSO-d₆) δ 7.37 (t, J=7.9 Hz, 1H), 7.12 (d, J=8.0 Hz, 1H), 6.66 (d, J=7.8 Hz, 1H), 5.89 (s, 1H), 3.23 (t, J=7.2 Hz, 2H), 1.75 (d, J=12.2 Hz, 2H), 1.70-1.59 (m, 3H), 1.57-1.50 (m, 2H), 1.45-1.35 (m, 1H), 1.27-1.13 (m, 3H), 0.98-0.89 (m, 2H). MS (ESI, m/z): 468, 470 (M+H)⁺

The following illustrative compound was made using the methods described in the Example immediately above, and substituting the appropriate reactants and/or reagents.

| | | Mass |
|---|---|---|
| Compound | Structure | [M + H]⁺ |
| 224 | 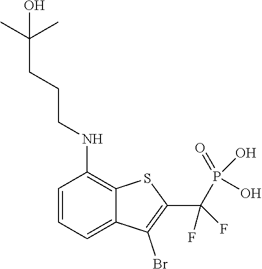 [SMILES: CC(C)(O)CCCNc1cccc2c(Br)c(C(F)(F)P(=O)(O)O)sc12] | 458, 460 |

### Example 16

### Preparation of Compound 225

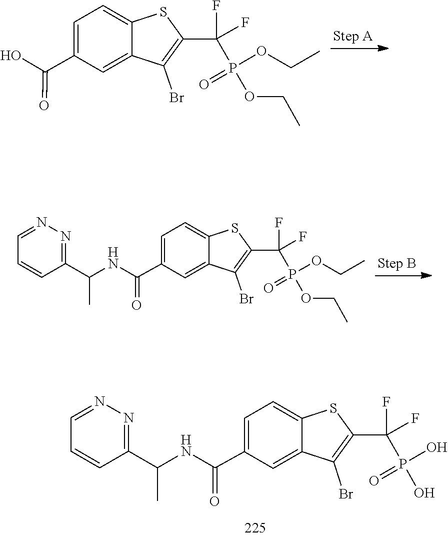 [SMILES: CCOP(=O)(OCC)C(F)(F)c1sc2ccc(C(=O)O)cc2c1Br ; CCOP(=O)(OCC)C(F)(F)c1sc2ccc(C(=O)NC(C)c3cccnn3)cc2c1Br ; CC(NC(=O)c1ccc2sc(C(F)(F)P(=O)(O)O)c(Br)c2c1)c1cccnn1]

### Step A: Synthesis of (R or S)-diethyl ((3-bromo-5-((1-(pyridazin-3-yl)ethyl)carbamoyl)benzo[b]thiophen-2-yl)difluoromethyl)phosphonate

HATU (172 mg, 0.451 mmol), and Hünig's base (146 mg, 1.13 mmol) were added to a mixture of compound Int-1 (100 mg, 0.226 mmol) in DMF (2.3 mL). The mixture was allowed to stir at room temperature for 5 minutes and then 1-(pyridazin-3-yl)ethan-1-amine (56 mg, 0.45 mmol) was added to the mixture. The mixture was allowed to stir at room temperature for 30 minutes. The mixture was filtered, and directly purified using reverse phase HPLC (eluting acetonitrile/water gradient with 0.1% TFA modifier) to provide the product as a mixture isomers. The mixture of isomers was resolved by chiral SFC (Chiralpak IA column, 21×250 mm, 5 μm; eluting 45% MeOH in CO2) to provide (R or S)-diethyl ((3-bromo-5-((1-(pyridazin-3-yl)ethyl)carbamoyl)benzo[b]thiophen-2-yl)difluoromethyl)phosphonate as the second eluting (of two) peak. MS (ESI, m/z): 548, 550 (M+H)⁺

### Step B: Synthesis of (R or S)-((3-bromo-5-((1-(pyridazin-3-yl)ethyl)carbamoyl)benzo[b]thiophen-2-yl)difluoromethyl)phosphonic acid

TMS-Br (355 μl, 2.74 mmol) was added to a mixture of diethyl (R or S)-((3-bromo-5-((1-(pyridazin-3-yl)ethyl)carbamoyl)benzo[b]thiophen-2-yl)difluoromethyl)phosphonate (50 mg, 0.091 mmol) in DCM (912 μl). The mixture was allowed to stir at room temperature for 1 day. MeOH (1 mL) was added and the mixture was filtered. The filtrate was purified using reverse phase HPLC (eluting acetonitrile/water with 0.1% TFA modifier) to provide (R or S)-((3-bromo-5-((1-(pyridazin-3-yl)ethyl)carbamoyl)benzo[b]thiophen-2-yl)difluoromethyl)phosphonic acid (compound 225). ¹H NMR (499 MHz, DMSO-d₆) δ 9.33 (d, J=7.3 Hz, 1H), 9.16 (d, J=4.8 Hz, 1H), 8.44 (s, 1H), 8.24 (d, J=8.5 Hz, 1H), 8.09 (d, J=8.5 Hz, 1H), 7.76 (d, J=8.4 Hz, 1H), 7.73-7.69 (m, 1H), 5.49-5.42 (m, 1H), 1.65 (d, J=7.1 Hz, 3H). MS (ESI, m/z): 492, 494 (M+H)⁺

### Example 17

### Preparation of Compound 226

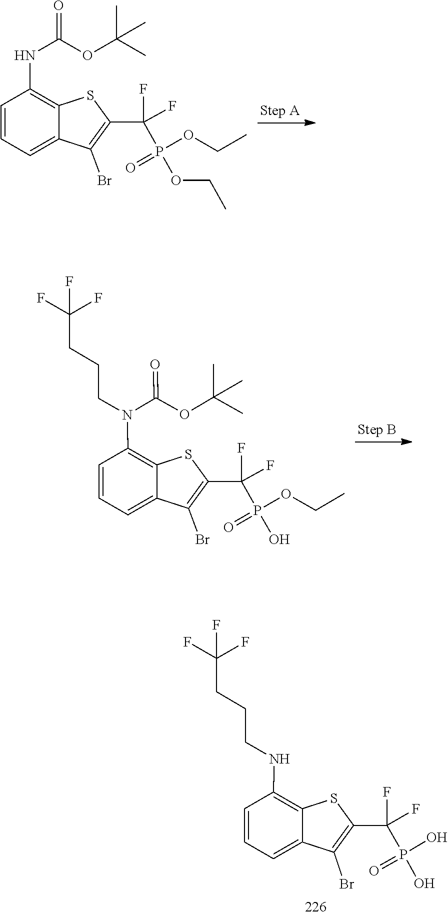 [SMILES: CCOP(=O)(OCC)C(F)(F)c1sc2c(NC(=O)OC(C)(C)C)cccc2c1Br ; CCOP(=O)(O)C(F)(F)c1sc2c(N(CCCC(F)(F)F)C(=O)OC(C)(C)C)cccc2c1Br ; O=P(O)(O)C(F)(F)c1sc2c(NCCCC(F)(F)F)cccc2c1Br]

### Step A: Synthesis of tert-butyl (3-bromo-2-((ethoxy(hydroxy)phosphoryl) difluoromethyl) benzo[b]thiophen-7-yl)(4,4,4-trifluorobutyl) carbamate

Sodium hydride (13 mg, 0.33 mmol) was added to a mixture of tert-butyl (3-bromo-2-((diethoxyphosphoryl)difluoromethyl)benzo[b]thiophen-7-yl)carbamate (40 mg, 0.082 mmol), and 4-bromo-1,1,1-trifluorobutane (31 mg, 0.17 mmol) in DMF (1.0 mL) at room temperature. The mixture was allowed to stir at room temperature for 2 hours. The mixture was concentrated in vacuo to provide tert-butyl (3-bromo-2-((ethoxy(hydroxy)phosphoryl)difluoromethyl) benzo[b]thiophen-7-yl)(4,4,4-trifluorobutyl)carbamate, which was used in the next step without purification. MS (ESI, m/z): 540, 542 (M+H-tBu)+

### Step B: Synthesis of ((3-bromo-7-((4,4,4-trifluorobutyl)amino)benzo[b]thiophen-2-yl)difluoromethyl)phosphonic acid

TMS-Br (1.07 mL, 8.22 mmol) was added to a mixture of tert-butyl (3-bromo-2-((ethoxy(hydroxy)phosphoryl)difluoromethyl)benzo[b]thiophen-7-yl)(4,4,4-trifluorobutyl)carbamate (49 mg, 0.082 mmol) in DMF (2.0 mL) at room temperature. The mixture was allowed to stir at room temperature for 3 days. The mixture was cooled to 0° C. and quenched with methanol (2 mL). The mixture was allowed to stir for 30 minutes at room temperature, and then concentrated in vacuo, and the resulting residue was taken up in DMF (3 mL), filtered, and purified using reverse phase HPLC (eluting acetonitrile in water, with 0.1% TFA modifier) to provide ((3-bromo-7-((4,4,4-trifluorobutyl)amino)benzo[b]thiophen-2-yl)difluoromethyl)phosphonic acid (compound 226). ¹H NMR (499 MHz, DMSO-d₆) δ 7.39 (t, J=7.9 Hz, 1H), 7.16 (d, J=8.0 Hz, 1H), 6.71 (d, J=7.8 Hz, 1H), 3.31 (t, J=6.8 Hz, 2H), 2.44-2.33 (m, 2H), 1.88-1.81 (m, 2H). MS (ESI, m/z): 468, 470 (M+H)⁺

### Example 18

### Preparation of Compound 227

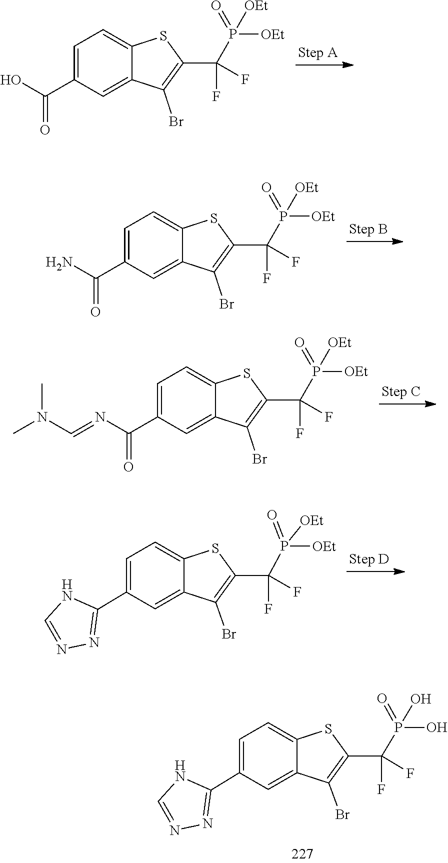

### Step A: Synthesis of diethyl ((3-bromo-5-carbamoylbenzo[b]thiophen-2-yl)difluoromethyl)phosphonate

A mixture of 3-bromo-2-((diethoxyphosphoryl)difluoromethyl)benzo[b]thiophene-5-carboxylic acid (50 mg, 0.11 mmol) in thionyl chloride (0.5 mL, 6.9 mmol) was allowed to stir at 50° C. for 1 hour then cooled to room temperature, and concentrated in vacuo. The resulting residue was taken up in ammonia (0.4 M in 1,4-dioxane, 1.0 mL, 0.4 mmol), and stirred at room temperature for 2 hours. The reaction was diluted with water (100 mL), and extracted with EtOAc (3×100 mL). The combined organic extracts were washed with brine (100 mL), dried over anhydrous Na₂SO₄ and concentrated in vacuo. The resulting residue was purified using reverse phase HPLC (eluting acetonitrile in water) to provide diethyl ((3-bromo-5-carbamoylbenzo[b]thiophen-2-yl)difluoromethyl)phosphonate. MS (ESI, m/z): 442, 444 (M+H)⁺

### Step B: Synthesis of diethyl (E)-((3-bromo-5-(((dimethylamino)methylene) carbamoyl)benzo[b]thiophen-2-yl)difluoromethyl)phosphonate

A mixture of diethyl ((3-bromo-5-carbamoylbenzo[b]thiophen-2-yl)difluoromethyl)phosphonate (34 mg, 0.077 mmol), and 1,1-dimethoxy-N,N-dimethylmethanamine (4 mL, 3 mmol) was allowed to stir for 2 hours at 80° C. under argon. The reaction mixture was concentrated in vacuo to provide diethyl (E)-((3-bromo-5-(((dimethylamino)methylene)carbamoyl)benzo[b]thiophen-2-yl)difluoromethyl)phosphonate, which was used in the next step without purification. MS (ESI, m/z): 497, 499 (M+H)⁺

### Step C: Synthesis of diethyl ((3-bromo-5-(4H-1,2,4-triazol-3-yl)benzo[b]thiophen-2-yl)difluoromethyl)phosphonate

To a mixture of diethyl (E)-((3-bromo-5-(((dimethylamino)methylene)carbamoyl)benzo[b]thiophen-2-yl)difluoromethyl)phosphonate (28 mg, 0.056 mmol) in acetic acid (0.8 mL) was added hydrazine hydrate (65% in water) (0.027 mL, 0.56 mmol) at room temperature under argon. The resulting solution was allowed to stir for 2 hours at 90° C. The reaction was cooled to room temperature, and was directly purified using reverse phase HPLC (eluting acetonitrile in water) to provide diethyl ((3-bromo-5-(4H-1,2,4-triazol-3-yl)benzo[b]thiophen-2-yl)difluoromethyl)phosphonate. MS (ESI, m/z): 466, 468 (M+H)⁺

### Step D: Synthesis of ((3-bromo-5-(4H-1,2,4-triazol-3-yl)benzo[b]thiophen-2-yl)difluoromethyl)phosphonic acid

To a solution of diethyl ((3-bromo-5-(4H-1,2,4-triazol-3-yl)benzo[b]thiophen-2-yl)difluoromethyl)phosphonate (18 mg, 0.039 mmol) in DMF (0.2 mL) was added TMS-Br (0.15 mL, 1.2 mmol) under argon. The resulting solution was allowed to stir for 1.5 hours at 60° C. The reaction was cooled to room temperature, quenched by addition of EtOH (0.5 mL), and concentrated in vacuo. The resulting residue was purified using reverse phase HPLC (eluting acetonitrile in water with NH₄HCO₃ modifier) to provide ((3-bromo-5-(4H-1,2,4-triazol-3-yl)benzo[b]thiophen-2-yl)difluoromethyl)phosphonic acid (compound 227). ¹H NMR: (300 MHz, DMSO-d₆) δ 8.49 (s, 1H), 8.47 (s, 1H), 8.12 (s, 2H). MS (ESI, m/z): 408, 410 (M−H)⁻

### Example 19

### Preparation of Compound 228

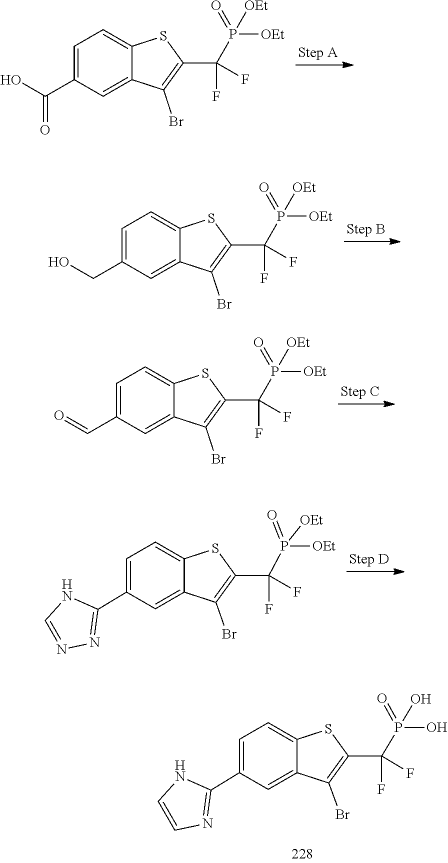

### Step A: Synthesis of diethyl ((3-bromo-5-(hydroxymethyl)benzo[b]thiophen-2-yl)difluoromethyl)phosphonate

To a mixture of 3-bromo-2-((diethoxyphosphoryl)difluoromethyl)benzo[b]thiophene-5-carboxylic acid (2000 mg, 4.5 mmol) in THF (5 mL) was added borane (1M in THF) (27 mL, 27 mmol) at 0° C. under argon. The resulting mixture was allowed to stir for 3 hours at room temperature. The reaction was quenched with water (15 mL), and concentrated in vacuo. The resulting residue was purified using reverse phase HPLC (eluting acetonitrile in water) to provide diethyl ((3-bromo-5-(hydroxymethyl)benzo[b]thiophen-2-yl)difluoromethyl)phosphonate. MS (ESI, m/z): 429, 431 (M+H)⁺

### Step B: Synthesis of diethyl ((3-bromo-5-formylbenzo[b]thiophen-2-yl)difluoromethyl)phosphonate

To a mixture of diethyl ((3-bromo-5-(hydroxymethyl)benzo[b]thiophen-2-yl)difluoromethyl)phosphonate (1400 mg, 3.3 mmol) in DCM (20 mL) was added Dess-Martin periodinane (2800 mg, 6.5 mmol) at 0° C. under argon. The resulting mixture was allowed to stir for 2 hours at room temperature. The reaction was quenched with saturated aqueous sodium bicarbonate (30 mL), and extracted with EtOAc (3×50 mL). The combined organic extracts were washed with brine (60 mL), dried over anhydrous Na₂SO₄ and concentrated in vacuo. The resulting residue was purified using silica gel chromatography (eluting ethyl acetate in petroleum ether) to provide diethyl ((3-bromo-5-formylbenzo[b]thiophen-2-yl)difluoromethyl)phosphonate. MS (ESI, m/z): 427, 429 (M+H)⁺

### Step C: Synthesis of diethyl ((3-bromo-5-(1H-imidazol-2-yl)benzo[b]thiophen-2-yl)difluoromethyl)phosphonate

To a mixture of diethyl ((3-bromo-5-formylbenzo[b]thiophen-2-yl)difluoromethyl)phosphonate (300 mg, 0.7 mmol) in MeOH (0.3 mL) was added a suspension of ammonium bicarbonate (110 mg, 1.4 mmol), and oxalaldehyde (40% in water) (0.09 mL, 0.07 mmol) in water (0.3 mL) at 0° C. under argon. The resulting solution was allowed to stir for 24 hours at room temperature. The reaction was directly purified using reverse phase HPLC (eluting acetonitrile in water) to provide diethyl ((3-bromo-5-(1H-imidazol-2-yl)benzo[b]thiophen-2-yl)difluoromethyl)phosphonate. MS (ESI, m/z): 465, 467 (M+H)⁺

### Step D: Synthesis of ((3-bromo-5-(1H-imidazol-2-yl)benzo[b]thiophen-2-yl)difluoromethyl)phosphonic acid

To a solution of diethyl ((3-bromo-5-(1H-imidazol-2-yl)benzo[b]thiophen-2-yl)difluoromethyl)phosphonate (35 mg, 0.075 mmol) in DMF (0.3 mL) was added TMS-Br (0.15 mL, 1.2 mmol) under argon. The resulting solution was allowed to stir for 1.5 hours at 60° C. The reaction was cooled to room temperature, quenched by addition of EtOH (0.3 mL), and concentrated in vacuo. The resulting residue was purified using reverse phase HPLC (eluting acetonitrile in water with NH₄HCO₃ modifier) to provide ((3-bromo-5-(1H-imidazol-2-yl)benzo[b]thiophen-2-yl)difluoromethyl)phosphonic acid (compound 228). ¹H NMR: (400 MHz, DMSO-d₆) δ 8.28 (s, 1H), 8.02 (d, J=8.5 Hz, 1H), 7.96 (d, J=8.6 Hz, 1H), 7.25 (s, 2H). MS (ESI, m/z): 409, 411 (M+H)⁺

### Example 20

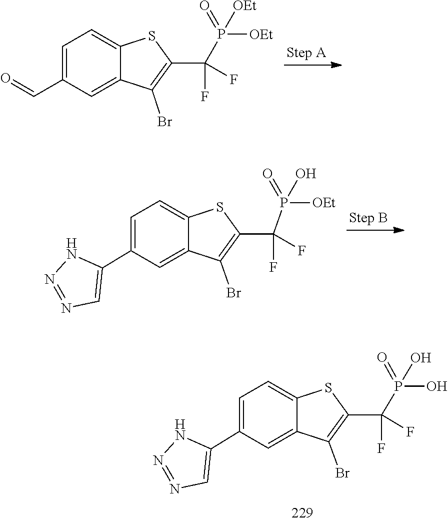 [SMILES: CCOP(=O)(OCC)C(F)(F)c1sc2ccc(C=O)cc2c1Br ; CCOP(=O)(O)C(F)(F)c1sc2ccc(-c3cnn[nH]3)cc2c1Br ; O=P(O)(O)C(F)(F)c1sc2ccc(-c3cnn[nH]3)cc2c1Br]

### Step A: Synthesis of ethyl hydrogen ((3-bromo-5-(1H-1,2,3-triazol-5-yl)benzo[b]thiophen-2-yl)difluoromethyl)phosphonate

To a mixture of diethyl ((3-bromo-5-formylbenzo[b]thiophen-2-yl)difluoromethyl)phosphonate (200 mg, 0.47 mmol) in DMSO (4 mL) was added nitromethane (0.038 mL, 0.70 mmol), sodium azide (37 mg, 0.56 mmol), and copper ferrite (5.6 mg, 0.023 mmol) at room temperature under argon. The mixture was subjected to microwave irradiation for 5 minutes at 120° C. The reaction was directly purified using reverse phase HPLC (eluting acetonitrile in water with NH₄HCO₃ modifier) to provide ethyl hydrogen ((3-bromo-5-(1H-1,2,3-triazol-5-yl)benzo[b]thiophen-2-yl)difluoromethyl)phosphonate. MS (ESI, m/z): 438, 440 (M+H)⁺

### Step B: Synthesis of ((3-bromo-5-(1H-1,2,3-triazol-5-yl)benzo[b]thiophen-2-yl)difluoromethyl)phosphonic acid

To a solution of ethyl hydrogen ((3-bromo-5-(1H-1,2,3-triazol-5-yl)benzo[b]thiophen-2-yl)difluoromethyl)phosphonate (18 mg, 0.041 mmol) in DMF (0.2 mL) was added TMS-Br (0.085 mL, 0.64 mmol) under argon. The resulting solution was allowed to stir for 1.5 hours at 60° C. The reaction was cooled to room temperature, quenched by addition of EtOH (0.3 mL), and concentrated in vacuo. The resulting residue was purified using reverse phase HPLC (eluting acetonitrile in water with NH₄HCO₃ modifier) to provide ((3-bromo-5-(1H-1,2,3-triazol-5-yl)benzo[b]thiophen-2-yl)difluoromethyl)phosphonic acid (compound 229). ¹H NMR (400 MHz, D₂O) δ 7.95 (s, 1H), 7.86 (s, 1H), 7.77 (d, J=8.4 Hz, 1H), 7.58 (d, J=8.3 Hz, 1H). MS (ESI, m/z): 410, 412 (M+H)⁺

### Example 21

### Preparation of Compound 230

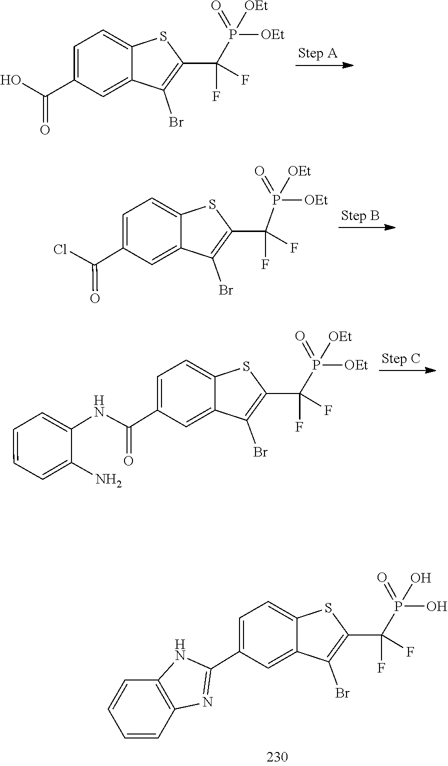 [SMILES: CCOP(=O)(OCC)C(F)(F)c1sc2ccc(C(=O)O)cc2c1Br ; CCOP(=O)(OCC)C(F)(F)c1sc2ccc(C(=O)Cl)cc2c1Br ; CCOP(=O)(OCC)C(F)(F)c1sc2ccc(C(=O)Nc3ccccc3N)cc2c1Br ; O=P(O)(O)C(F)(F)c1sc2ccc(-c3nc4ccccc4[nH]3)cc2c1Br]

### Step A: Synthesis of diethyl ((3-bromo-5-(chlorocarbonyl)benzo[b]thiophen-2-yl)difluoromethyl)phosphonate

A mixture of 3-bromo-2-((diethoxyphosphoryl)difluoromethyl)benzo[b]thiophene-5-carboxylic acid (400 mg, 0.09 mmol) in thionyl chloride (4 mL, 55 mmol) was allowed to stir for 1 hour at 80° C. under argon. The reaction mixture was cooled to room temperature, and concentrated in vacuo to provide diethyl ((3-bromo-5-(chlorocarbonyl)benzo[b]thiophen-2-yl)difluoromethyl)phosphonate, which was used in next step without purification.

### Step B: Synthesis of diethyl ((5-((2-aminophenyl)carbamoyl)-3-bromobenzo[b]thiophen-2-yl)difluoromethyl)phosphonate

To a mixture of diethyl ((3-bromo-5-(chlorocarbonyl)benzo[b]thiophen-2-yl)difluoromethyl)phosphonate (400 mg, 0.87 mmol) in DCM (4 mL) was added benzene-1,2-diamine (110 mg, 1.0 mmol), and TEA (0.22 mL, 1.6 mmol) at room temperature under argon. The resulting mixture was allowed to stir at room temperature for 2 hours. The reaction was quenched with water (10 mL), and extracted with EtOAc (3×15 mL). The combined organic extracts were washed with brine (30 mL), dried over anhydrous Na₂SO₄ and concentrated in vacuo. The resulting residue was purified using silica gel chromatography (eluting ethyl acetate in petroleum ether) to provide diethyl ((5-((2-aminophenyl)carbamoyl)-3-bromobenzo[b]thiophen-2-yl)difluoromethyl)phosphonate. MS (ESI, m/z): 533, 535 (M+H)⁺

### Step C: Synthesis of ((5-(1H-benzo[d]imidazol-2-yl)-3-bromobenzo[b]thiophen-2-yl)difluoromethyl)phosphonic acid

To a mixture of diethyl ((5-((2-aminophenyl)carbamoyl)-3-bromobenzo[b]thiophen-2-yl)difluoromethyl)phosphonate (30 mg, 0.056 mmol) in xylene (0.3 mL) was added 4-methylbenzenesulfonic acid (19 mg, 0.11 mmol) at room temperature under argon. The resulting mixture was allowed to stir for 1 hour at 140° C. The reaction was directly purified using reverse phase HPLC (eluting acetonitrile in water with NH₄HCO₃ modifier) to provide ((5-(1H-benzo[d]imidazol-2-yl)-3-bromobenzo[b]thiophen-2-yl)difluoromethyl)phosphonic acid (compound 230). ¹H NMR (300 MHz, DMSO-d₆) δ 8.63 (s, 1H), 8.29 (d, J=8.5 Hz, 1H), 8.18 (d, J=8.5 Hz, 1H), 7.72-7.57 (m, 2H), 7.29-7.19 (m, 2H). MS (ESI, m/z): 459, 461 (M+H)⁺

### Example 22

### Preparation of Compound 231

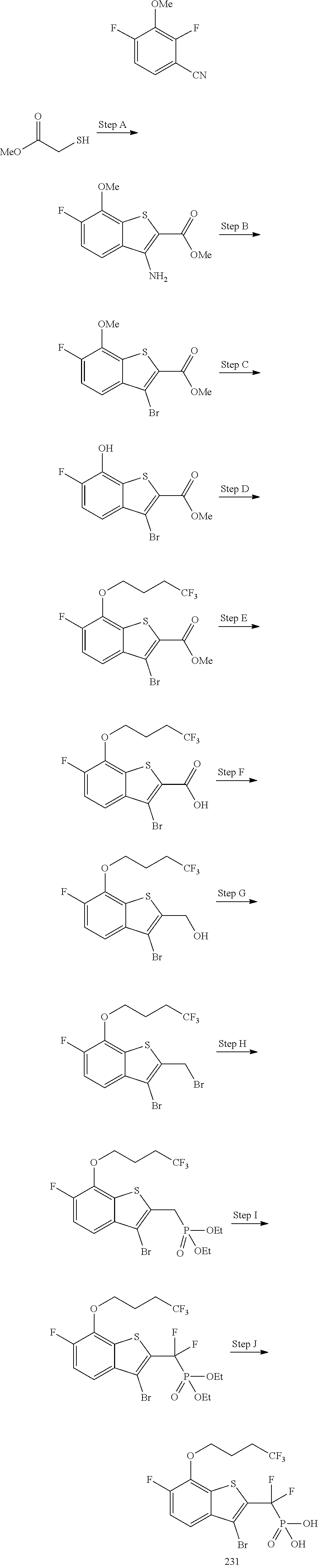

### Step A: Synthesis of methyl 3-amino-6-fluoro-7-methoxybenzo[b]thiophene-2-carboxylate

To a mixture of 2,4-difluoro-3-methoxybenzonitrile (4600 mg, 27 mmol), and methyl 2-mercaptoacetate (2900 mg, 27 mmol) in DMF (20 mL) was added potassium tert-butoxide (3700 mg, 33 mmol) at 0° C. The mixture was warmed to room temperature, and stirred for 1 hour. The reaction was poured into ice water (300 mL), and extracted with EtOAc (3×300 mL). The combined organic extracts were washed with brine (300 mL), dried over anhydrous Na2SO₄ and concentrated in vacuo. The resulting residue was purified using silica gel chromatography (eluting ethyl acetate in petroleum ether) to provide methyl 3-amino-6-fluoro-7-methoxybenzo[b]thiophene-2-carboxylate. MS (ESI, m/z): 254 (M−H)⁻

### Step B: Synthesis of methyl 3-bromo-6-fluoro-7-methoxybenzo[b]thiophene-2-carboxylate

To a mixture of copper (II) bromide (3300 mg, 15 mmol), and tert-butyl nitrite (1900 mg, 18 mmol) in acetonitrile (80 mL) was added a solution of methyl 3-amino-6-fluoro-7-methoxybenzo[b]thiophene-2-carboxylate (3100 mg, 12 mmol) in acetonitrile (40 mL). The resulting solution was allowed to stir for 2 hours at 65° C. The mixture was cooled to room temperature then poured into saturated aqueous NH₄Cl (500 mL), and extracted with EtOAc (3×500 mL). The combined organic extracts were washed with brine (500 mL), dried over anhydrous Na₂SO₄ and concentrated in vacuo. The resulting residue was purified using silica gel chromatography (eluting ethyl acetate in petroleum ether) to provide methyl 3-bromo-6-fluoro-7-methoxybenzo[b]thiophene-2-carboxylate. ¹H NMR (300 MHz, CDCl₃) δ 7.61 (dd, J=8.9, 4.0 Hz, 1H), 7.28 (dd, J=11.8, 8.9 Hz, 1H), 4.16 (d, J=2.6 Hz, 3H), 3.97 (s, 3H).

### Step C: Synthesis of methyl 3-bromo-6-fluoro-7-hydroxybenzo[b]thiophene-2-carboxylate

To a mixture of methyl 3-bromo-6-fluoro-7-methoxybenzo[b]thiophene-2-carboxylate (3000 mg, 9.4 mmol) in DCM (30 mL) was added boron tribromide (1.0 M in DCM, 94 mL, 94 mmol) at −78° C. The mixture was allowed to stir for 3 hours at 0° C. The reaction was quenched by addition of MeOH (50 mL), and concentrated in vacuo. The resulting residue was purified using silica gel chromatography (eluting ethyl acetate in petroleum ether) to provide methyl 3-bromo-6-fluoro-7-hydroxybenzo[b]thiophene-2-carboxylate. MS (ESI, m/z): 305, 307 (M+H)⁺

### Step D: Synthesis of methyl 3-bromo-6-fluoro-7-(4,4,4-trifluorobutoxy)benzo[b]thiophene-2-carboxylate

To a mixture of methyl 3-bromo-6-fluoro-7-hydroxybenzo[b]thiophene-2-carboxylate (2400 mg, 7.9 mmol), and 4-bromo-1,1,1-trifluorobutane (1800 mg, 9.4 mmol) in DMF (24 mL) was added potassium carbonate (2200 mg, 16 mmol) at room temperature. The resulting mixture was allowed to stir for 2 hours at room temperature. The reaction was diluted with water (200 mL) and extracted with EtOAc (3×200 mL). The combined organic extracts were washed with brine (200 mL), dried over anhydrous Na₂SO₄ and concentrated in vacuo. The resulting residue was purified using silica gel chromatography (eluting ethyl acetate in petroleum ether) to provide methyl 3-bromo-6-fluoro-7-(4,4,4-trifluorobutoxy)benzo[b]thiophene-2-carboxylate. ¹H NMR (400 MHz, CDCl₃) δ 7.59-7.54 (m, 1H), 7.26-7.19 (m, 1H), 4.31 (t, J=5.9 Hz, 2H), 3.91 (s, 3H), 2.42-2.26 (m, 2H), 2.09-1.96 (m, 2H).

### Step E: Synthesis of 3-bromo-6-fluoro-7-(4,4,4-trifluorobutoxy)benzo[b]thiophene-2-carboxylic acid

To a mixture of methyl 3-bromo-6-fluoro-7-(4,4,4-trifluorobutoxy)benzo[b]thiophene-2-carboxylate (2400 mg, 5.8 mmol) in THF (10 mL), and MeOH (10 mL) was added aqueous sodium hydroxide (6 M, 19 mL, 120 mmol) at room temperature. The mixture was allowed to stir for 4 hours at 60° C. The reaction mixture was cooled to room temperature, and pH was adjusted between 3-4 with aqueous HCl (6 M). The mixture was diluted with water (200 mL), and extracted with EtOAc (3×300 mL). The combined organic extracts were washed with brine (300 mL), dried over anhydrous Na₂SO₄ and concentrated in vacuo to provide 3-bromo-6-fluoro-7-(4,4,4-trifluorobutoxy)benzo[b]thiophene-2-carboxylic acid, which was used in the next step without purification. MS (ESI, m/z): 399, 401 (M−H)⁻

### Step F: Synthesis of (3-bromo-6-fluoro-7-(4,4,4-trifluorobutoxy)benzo[b]thiophen-2-yl)methanol

To a mixture of 3-bromo-6-fluoro-7-(4,4,4-trifluorobutoxy)benzo[b]thiophene-2-carboxylic acid (2400 mg, 6.0 mmol) in THF (36 mL) was added borane (1M in THF) (36 mL, 36 mmol) at room temperature. The mixture was allowed to stir for 4 hours at room temperature. The reaction mixture was quenched by addition of MeOH (50 mL), and concentrated in vacuo. The resulting residue was purified using silica gel column chromatography (eluting ethyl acetate in petroleum ether) to provide (3-bromo-6-fluoro-7-(4,4,4-trifluorobutoxy)benzo[b]thiophen-2-yl)methanol. MS (ESI, m/z): 385, 387 (M−H)⁻

### Step G: Synthesis of 3-bromo-2-(bromomethyl)-6-fluoro-7-(4,4,4-trifluorobutoxy)benzo[b]thiophene

To a mixture of (3-bromo-6-fluoro-7-(4,4,4-trifluorobutoxy)benzo[b]thiophen-2-yl)methanol (1900 mg, 4.9 mmol) in DCM (50 mL) was added phosphorus tribromide (2700 g, 9.8 mmol) at room temperature. The mixture was allowed to stir for 2 hours at room temperature. The mixture was concentrated under pressure. The resulting residue was purified using silica gel column chromatography (eluting ethyl acetate in petroleum ether) to provide 3-bromo-2-(bromomethyl)-6-fluoro-7-(4,4,4-trifluorobutoxy)benzo[b]thiophene. ¹H NMR: (400 MHz, CDCl₃) δ 7.43 (dd, J=8.8, 4.0 Hz, 1H), 7.24 (dd, J=11.7, 8.8 Hz, 1H), 4.76 (s, 2H), 4.36 (t, J=6.0 Hz, 2H), 2.49-2.33 (m, 2H), 2.14-2.03 (m, 2H).

### Step H: Synthesis of diethyl ((3-bromo-6-fluoro-7-(4,4,4-trifluorobutoxy)benzo[b]thiophen-2-yl)methyl)phosphonate

To a mixture of 3-bromo-2-(bromomethyl)-6-fluoro-7-(4,4,4-trifluorobutoxy)benzo[b]thiophene (500 mg, 1.1 mmol) in 1,4-dioxane (10 mL) was added triethyl phosphite (370 mg, 2.2 mmol) at room temperature. The mixture was allowed to stir for 3 hours at 110° C. The mixture was cooled to room temperature, and concentrated in vacuo. The resulting residue was purified using reverse phase HPLC (eluting acetonitrile in water) to provide diethyl ((3-bromo-6-fluoro-7-(4,4,4-trifluorobutoxy)benzo[b]thiophen-2-yl)methyl)phosphonate. MS (ESI, m/z): 507, 509 (M+H)⁺

### Step I: Synthesis of diethyl ((3-bromo-6-fluoro-7-(4,4,4-trifluorobutoxy)benzo[b]thiophen-2-yl)difluoromethyl)phosphonate

To a mixture of diethyl ((3-bromo-6-fluoro-7-(4,4,4-trifluorobutoxy)benzo[b]thiophen-2-yl)methyl)phosphonate (100 mg, 0.2 mmol) in THF (2 mL) was added sodium bis(trimethylsilyl)amide (1.9 M in THF) (0.26 mL, 0.49 mmol) at −78° C. The mixture was allowed to stir for 1 hour at −78° C. To the reaction was added a solution of N-fluorobenzenesulfonimide (170 mg, 0.53 mmol) in THF (1 mL) at −78° C. The mixture was allowed to stir for 1 hour at −78° C. The reaction mixture was quenched by addition of saturated aqueous NH₄Cl (50 mL), and extracted with EtOAc (3×50 mL). The combined organic extracts were washed with brine (100 mL), dried over anhydrous Na₂SO₄ and concentrated in vacuo. The resulting residue was purified using silica gel chromatography (eluting ethyl acetate in petroleum ether) to provide diethyl ((3-bromo-6-fluoro-7-(4,4,4-trifluorobutoxy)benzo[b]thiophen-2-yl)difluoromethyl)phosphonate. MS (ESI, m/z): 543, 545 (M+H)⁺

### Step J: Synthesis of ((3-bromo-6-fluoro-7-(4,4,4-trifluorobutoxy)benzo[b]thiophen-2-yl)difluoromethyl)phosphonic acid

To a mixture of diethyl ((3-bromo-6-fluoro-7-(4,4,4-trifluorobutoxy)benzo[b]thiophen-2-yl)difluoromethyl)phosphonate (30 mg, 0.055 mmol) in DMF (0.42 mL) was added TMS-Br (0.22 mL, 1.7 mmol) at room temperature. The resulting solution was allowed to stir for 1.5 hours at 60° C. The reaction was cooled to room temperature, quenched by addition of EtOH (1 mL), and concentrated in vacuo. The resulting residue was purified using reverse phase HPLC (eluting acetonitrile in water with NH₄HCO₃ modifier) to provide ((3-bromo-6-fluoro-7-(4,4,4-trifluorobutoxy)benzo[b]thiophen-2-yl)difluoromethyl)phosphonic acid (compound 231). ¹H NMR (400 MHz, D₂O) δ 7.66 (dd, J=8.9, 4.1 Hz, 1H), 7.39 (dd, J=11.7, 8.9 Hz, 1H), 4.42 (t, J=6.2 Hz, 2H), 2.52-2.41 (m, 2H), 2.13-2.03 (m, 2H). MS (ESI, m/z): 485, 487 (M−H)⁻

### Example 23

### Preparation of Compound 232

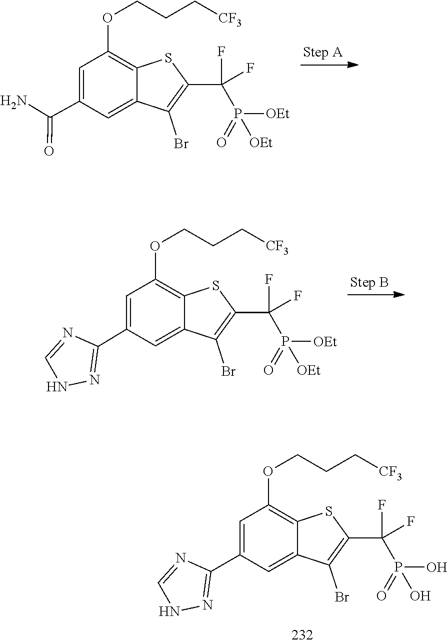 [SMILES: CCOP(=O)(OCC)C(F)(F)c1sc2c(OCCCC(F)(F)F)cc(C(N)=O)cc2c1Br ; CCOP(=O)(OCC)C(F)(F)c1sc2c(OCCCC(F)(F)F)cc(-c3nc[nH]n3)cc2c1Br ; O=P(O)(O)C(F)(F)c1sc2c(OCCCC(F)(F)F)cc(-c3nc[nH]n3)cc2c1Br]

### Step A: Synthesis of diethyl ((3-bromo-5-(1H-1,2,4-triazol-3-yl)-7-(4,4,4-trifluorobutoxy)benzo[b]thiophen-2-yl)difluoromethyl)phosphonate

A mixture of diethyl ((3-bromo-5-carbamoyl-7-(4,4,4-trifluorobutoxy)benzo[b]thiophen-2-yl)difluoromethyl)phosphonate (45 mg, 0.079 mmol) in 1,1-dimethoxy-N,N-dimethylmethanamine (0.5 mL, 0.079 mmol) was allowed to stir for 2 hours at 80° C. The reaction was concentrated in vacuo and the resulting residue was taken up in AcOH (0.5 mL). Hydrazine hydrate (0.04 mL, 0.8 mmol) was added at room temperature under argon. The resulting solution was allowed to stir for 2 hours at 90° C. The reaction was cooled to room temperature, diluted with water (20 mL), and extracted with EtOAc (3×30 mL). The combined organic extracts were washed with brine (60 mL), dried over anhydrous Na₂SO₄ and concentrated in vacuo. The resulting residue was purified using reverse phase HPLC (eluting acetonitrile in water) to provide diethyl ((3-bromo-5-(1H-1,2,4-triazol-3-yl)-7-(4,4,4-trifluorobutoxy)benzo[b]thiophen-2-yl)difluoromethyl)phosphonate. MS (ESI, m/z): 592, 594 (M+H)⁺

### Step B: Synthesis of ((3-bromo-5-(1H-1,2,4-triazol-3-yl)-7-(4,4,4-trifluorobutoxy)benzo[b]thiophen-2-yl)difluoromethyl)phosphonic acid

To a solution of diethyl ((3-bromo-5-(1H-1,2,4-triazol-3-yl)-7-(4,4,4-trifluorobutoxy)benzo[b]thiophen-2-yl)difluoromethyl)phosphonate (30 mg, 0.051 mmol) in DMF (0.3 mL) was added TMSBr (0.15 mL, 1.2 mmol) under argon. The resulting solution was allowed to stir for 1.5 hours at 60° C. The reaction was cooled to room temperature, quenched by addition of EtOH (0.3 mL), and concentrated in vacuo. The resulting residue was purified using reverse phase HPLC (eluting acetonitrile in water with NH₄HCO₃ modifier) to provide ((3-bromo-5-(1H-1,2,4-triazol-3-yl)-7-(4,4,4-trifluorobutoxy)benzo[b]thiophen-2-yl)difluoromethyl)phosphonic acid (compound 232). ¹H NMR (400 MHz, deuterium oxide) δ 8.12 (s, 1H), 7.22 (s, 1H), 6.68 (s, 1H), 3.92 (t, J=6.1 Hz, 2H), 2.38-2.24 (m, 2H), 2.02-1.92 (m, 2H). MS (ESI, m/z): 536, 538 (M+H)⁺

### Example 24

### Preparation of Compound 233 Preparation of((3-bromo-5-cyano-7-(4,4,4-trifluorobutoxy)benzo[b]thiophen-2-yl)difluoromethyl)phosphonic acid

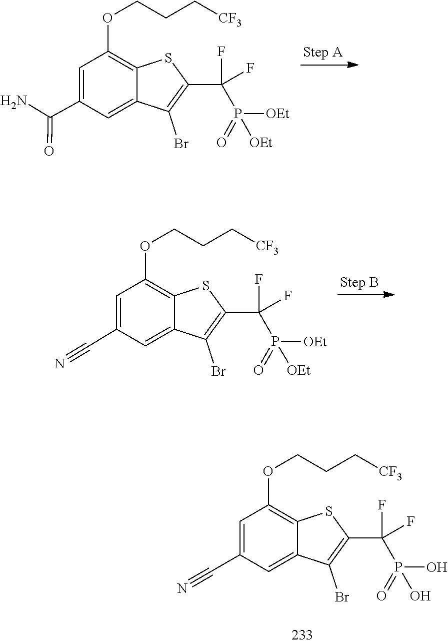 [SMILES: CCOP(=O)(OCC)C(F)(F)c1sc2c(OCCCC(F)(F)F)cc(C(N)=O)cc2c1Br ; CCOP(=O)(OCC)C(F)(F)c1sc2c(OCCCC(F)(F)F)cc(C#N)cc2c1Br ; N#Cc1cc(OCCCC(F)(F)F)c2sc(C(F)(F)P(=O)(O)O)c(Br)c2c1]

### Step A: Synthesis of diethyl ((3-bromo-5-cyano-7-(4,4,4-trifluorobutoxy)benzo[b]thiophen-2-yl)difluoromethyl)phosphonate

To a mixture of diethyl ((3-bromo-5-carbamoyl-7-(4,4,4-trifluorobutoxy)benzo[b]thiophen-2-yl)difluoromethyl)phosphonate (30 mg, 0.053 mmol) in DCM (0.3 mL) was added triethylamine (0.022 mL, 0.16 mmol), and trifluoromethanesulfonic anhydride (0.098 mL, 0.058 mmol) at 0° C. under argon. The reaction was allowed to stir for 2 hours at room temperature. The reaction was diluted with water (10 mL), and extracted with DCM (3×20 mL). The combined organic extracts were washed with brine (10 mL), dried over anhydrous Na₂SO₄ and concentrated in vacuo. The resulting residue was purified using Prep-TLC (developed by ethyl acetate/petroleum ether) to provide diethyl ((3-bromo-5-cyano-7-(4,4,4-trifluorobutoxy)benzo[b]thiophen-2-yl)difluoromethyl)phosphonate. MS (ESI, m/z): 550, 552 (M+H)⁺

### Step B: Synthesis of ((3-bromo-5-cyano-7-(4,4,4-trifluorobutoxy)benzo[b]thiophen-2-yl)difluoromethyl)phosphonic acid

To a solution of diethyl ((3-bromo-5-cyano-7-(4,4,4-trifluorobutoxy)benzo[b]thiophen-2-yl)difluoromethyl)phosphonate (18 mg, 0.033 mmol) in DMF (0.2 mL) was added TMS-Br (0.13 mL, 0.98 mmol) under argon. The resulting solution was allowed to stir for 1 hour at 40° C. The reaction was cooled to room temperature, quenched by addition of EtOH (0.1 mL), and concentrated in vacuo. The resulting residue was purified using reverse phase HPLC (eluting acetonitrile in water with NH₄HCO₃ modifier) to provide ((3-bromo-5-cyano-7-(4,4,4-trifluorobutoxy)benzo[b]thiophen-2-yl)difluoromethyl)phosphonic acid (compound 233). ¹H NMR (400 MHz, deuterium oxide) δ 7.85 (s, 1H), 7.18 (s, 1H), 4.29 (t, J=6.0 Hz, 2H), 2.52-2.35 (m, 2H), 2.17-2.07 (m, 2H). MS (ESI, m/z): 492, 494 (M−H)⁻

### Example 25

### Preparation of Compound 234

### Preparation of ((3-bromo-5-carbamoyl-7-(3-methoxy-3-methylbut-1-yn-1-yl)benzo[b]thiophen-2-yl)difluoromethyl)phosphonic acid

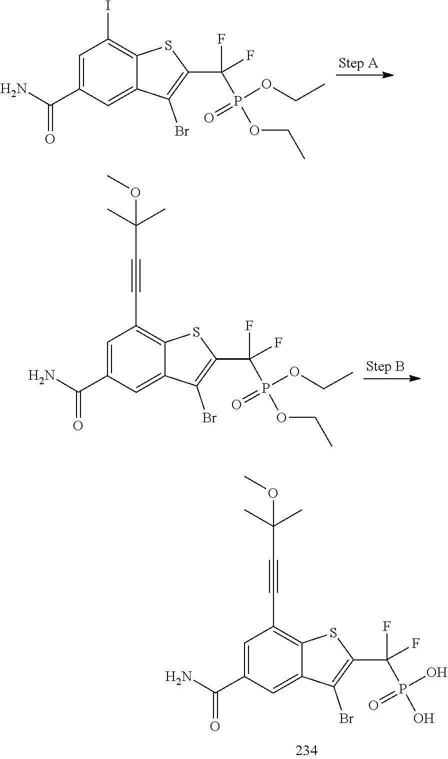 [SMILES: CCOP(=O)(OCC)C(F)(F)c1sc2c(I)cc(C(N)=O)cc2c1Br ; CCOP(=O)(OCC)C(F)(F)c1sc2c(C#CC(C)(C)OC)cc(C(N)=O)cc2c1Br ; COC(C)(C)C#Cc1cc(C(N)=O)cc2c(Br)c(C(F)(F)P(=O)(O)O)sc12]

### Step A: Synthesis of diethyl ((3-bromo-5-carbamoyl-7-(3-methoxy-3-methylbut-1-yn-1-yl)benzo[b]thiophen-2-yl)difluoromethyl)phosphonate

A mixture of Int-13 (25 mg, 0.044 mmol), and 3-methoxy-3-methylbut-1-yne (13 mg, 0.13 mmol), copper (I) iodide (3.4 mg, 0.018 mmol), bis(triphenylphosphine)palladium (II) dichloride (6.2 mg, 8.8 μmol), and triethylamine (12 μl, 0.088 mmol) was degassed with nitrogen. THF (0.44 mL was added and the mixture was allowed to stir at room temperature for 15 minutes. The mixture was filtered, and the filtrate was concentrated in vacuo to provide diethyl ((3-bromo-5-carbamoyl-7-(3-methoxy-3-methylbut-1-yn-1-yl)benzo[b]thiophen-2-yl)difluoromethyl)phosphonate, which was used in the next step without purification. MS (ESI, m/z): 538, 540 (M+H)⁺

### Step B: Synthesis of ((3-bromo-5-carbamoyl-7-(3-methoxy-3-methylbut-1-yn-1-yl)benzo[b]thiophen-2-yl)difluoromethyl)phosphonic acid

TMS-Br (114 μl, 0.880 mmol) was added to a mixture of diethyl ((3-bromo-5-carbamoyl-7-(3-methoxy-3-methylbut-1-yn-1-yl)benzo[b]thiophen-2-yl)difluoromethyl)phosphonate (24 mg, 0.044 mmol) in DMF (0.5 mL) at room temperature. The mixture was allowed to stir for 18 hours at room temperature, and then heated to 60° C. for 1 hour (to effect complete conversion). The mixture was diluted with DMSO (1 mL), then directly purified using reverse phase HPLC (eluting acetonitrile in water, with 0.1% TFA modifier) to provide ((3-bromo-5-carbamoyl-7-(3-methoxy-3-methylbut-1-yn-1-yl)benzo[b]thiophen-2-yl)difluoromethyl)phosphonic acid (compound 234). ¹H NMR (600 MHz, DMSO-d₆) δ 8.41 (s, 1H), 8.35 (s, 1H), 8.18 (s, 1H), 7.64 (s, 1H), 3.40 (s, 3H), 1.57 (s, 6H). MS (ESI, m/z): 482, 484 (M+H)⁺

### Example 26

### Preparation of Compound 235

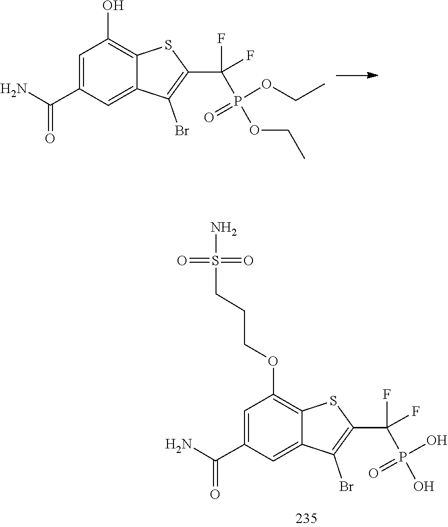 [SMILES: CCOP(=O)(OCC)C(F)(F)c1sc2c(O)cc(C(N)=O)cc2c1Br ; NC(=O)c1cc(OCCCS(N)(=O)=O)c2sc(C(F)(F)P(=O)(O)O)c(Br)c2c1]

A mixture of diethyl ((3-bromo-5-carbamoyl-7-hydroxybenzo[b]thiophen-2-yl)difluoromethyl)phosphonate (20 mg, 0.044 mmol), 3-bromopropane-1-sulfonamide (18 mg, 0.087 mmol), and potassium carbonate (9 mg, 0.07 mmol) in DMF (0.175 mL) was allowed to stir at room temperature for 18 hours. TMS-Br (113 μl, 0.873 mmol) was added to the mixture and the mixture was stirred and heated at 60° C. for 1 hour. The mixture was cooled to room temperature, and quenched with methanol (1 mL). The mixture was filtered, and the filtrate was purified using reverse phase HPLC (eluting acetonitrile in water, with 0.1% TFA modifier) to provide ((3-bromo-5-carbamoyl-7-(3-sulfamoylpropoxy)benzo[b]thiophen-2-yl)difluoromethyl)phosphonic acid (compound 235). ¹H NMR (600 MHz, DMSO-d₆) δ 8.29 (s, 1H), 8.05 (s, 1H), 7.61 (s, 1H), 7.56 (s, 1H), 6.92 (s, 2H), 4.42 (t, J=6.1 Hz, 2H), 3.23-3.19 (m, 3H), 2.29-2.23 (m, 2H). MS (ESI, m/z): 521, 523 (M−H)⁻

The following illustrative compounds were made using the methods described in the Example immediately above and substituting the appropriate reactants and/or reagents.

| | | Mass |
|---|---|---|
| Compound | Structure | [M + H]⁺ |
| 236 | 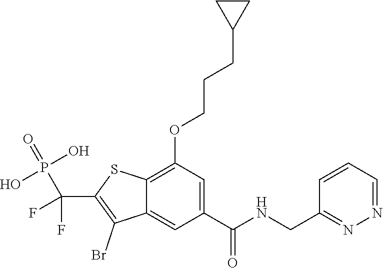 [SMILES: O=C(NCc1cccnn1)c1cc(OCCCC2CC2)c2sc(C(F)(F)P(=O)(O)O)c(Br)c2c1] | 576, 578 |
| 237 | 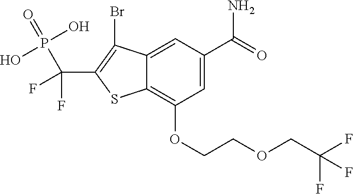 [SMILES: NC(=O)c1cc(OCCOCC(F)(F)F)c2sc(C(F)(F)P(=O)(O)O)c(Br)c2c1] | 528, 530 |
| 238 | 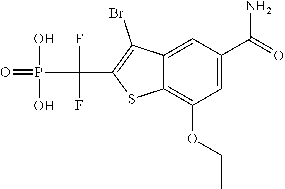 | 859 (2M + H)⁺ |
| 239 | 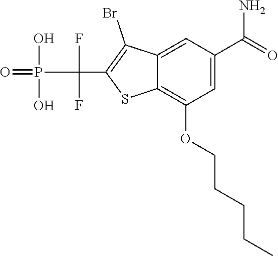 [SMILES: CCCCCOc1cc(C(N)=O)cc2c(Br)c(C(F)(F)P(=O)(O)O)sc12] | 472, 474 |
| 240 | 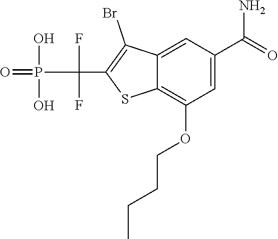 [SMILES: CCCCOc1cc(C(N)=O)cc2c(Br)c(C(F)(F)P(=O)(O)O)sc12] | 458, 460 |
| 241 | 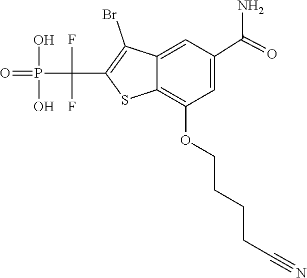 [SMILES: N#CCCCCOc1cc(C(N)=O)cc2c(Br)c(C(F)(F)P(=O)(O)O)sc12] | 483 |
| 242 | 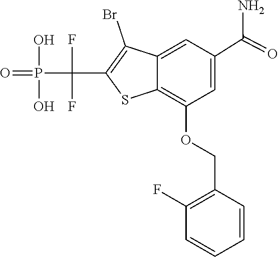 [SMILES: NC(=O)c1cc(OCc2ccccc2F)c2sc(C(F)(F)P(=O)(O)O)c(Br)c2c1] | 510, 512 |
| 243 | 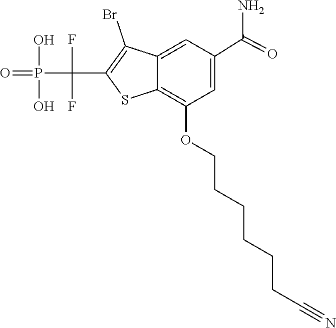 [SMILES: N#CCCCCCCOc1cc(C(N)=O)cc2c(Br)c(C(F)(F)P(=O)(O)O)sc12] | 511, 513 |
| 244 | 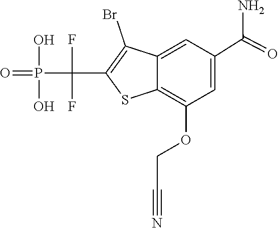 [SMILES: N#CCOc1cc(C(N)=O)cc2c(Br)c(C(F)(F)P(=O)(O)O)sc12] | 441, 443 |
| 245 | 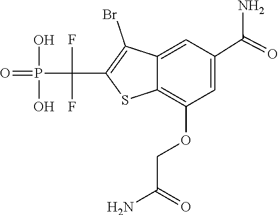 [SMILES: NC(=O)COc1cc(C(N)=O)cc2c(Br)c(C(F)(F)P(=O)(O)O)sc12] | 459, 461 |
| 246 | 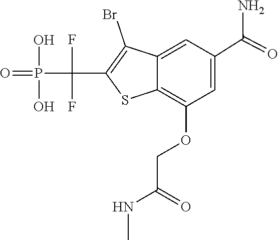 [SMILES: CNC(=O)COc1cc(C(N)=O)cc2c(Br)c(C(F)(F)P(=O)(O)O)sc12] | 473 |
| 247 | 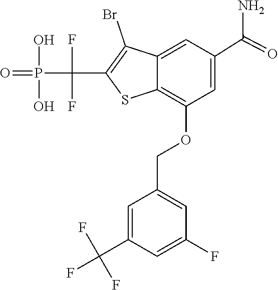 [SMILES: NC(=O)c1cc(OCc2cc(F)cc(C(F)(F)F)c2)c2sc(C(F)(F)P(=O)(O)O)c(Br)c2c1] | 578, 580 |
| 248 | 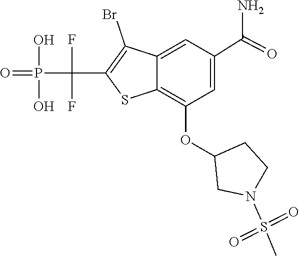 [SMILES: CS(=O)(=O)N1CCC(Oc2cc(C(N)=O)cc3c(Br)c(C(F)(F)P(=O)(O)O)sc23)C1] | 549, 551 |
| 249 | 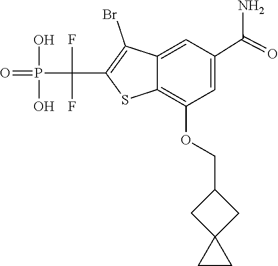 [SMILES: NC(=O)c1cc(OCC2CC3(CC3)C2)c2sc(C(F)(F)P(=O)(O)O)c(Br)c2c1] | 496, 498 |
| 250 | 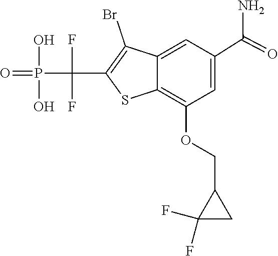 [SMILES: NC(=O)c1cc(OCC2CC2(F)F)c2sc(C(F)(F)P(=O)(O)O)c(Br)c2c1] | 492, 494 |
| 251 | 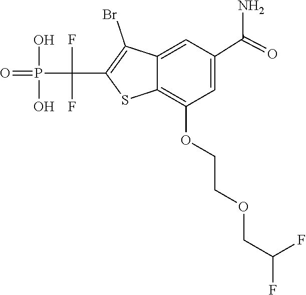 [SMILES: NC(=O)c1cc(OCCOCC(F)F)c2sc(C(F)(F)P(=O)(O)O)c(Br)c2c1] | 510, 512 |
| 252 | 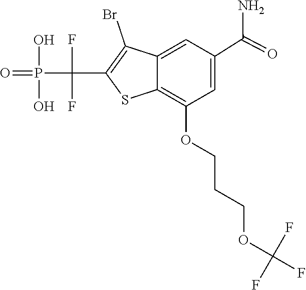 [SMILES: NC(=O)c1cc(OCCCOC(F)(F)F)c2sc(C(F)(F)P(=O)(O)O)c(Br)c2c1] | 528, 530 |
| 253 | 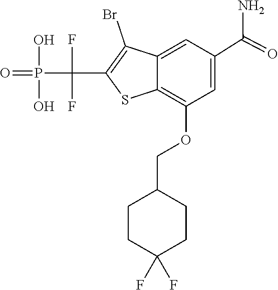 [SMILES: NC(=O)c1cc(OCC2CCC(F)(F)CC2)c2sc(C(F)(F)P(=O)(O)O)c(Br)c2c1] | 534, 536 |
| 254 | 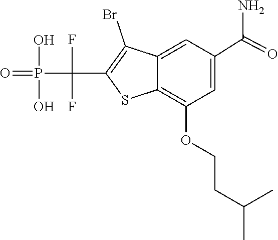 [SMILES: CC(C)CCOc1cc(C(N)=O)cc2c(Br)c(C(F)(F)P(=O)(O)O)sc12] | 472, 474 |
| 255 | 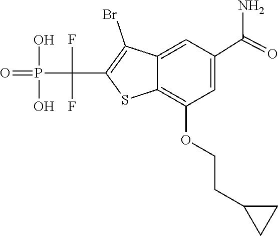 [SMILES: NC(=O)c1cc(OCCC2CC2)c2sc(C(F)(F)P(=O)(O)O)c(Br)c2c1] | 470, 472 |
| 256 | 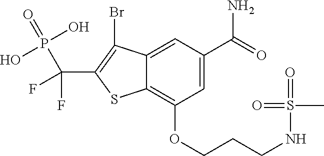 [SMILES: CS(=O)(=O)NCCCOc1cc(C(N)=O)cc2c(Br)c(C(F)(F)P(=O)(O)O)sc12] | 537, 539 |
| 257 | 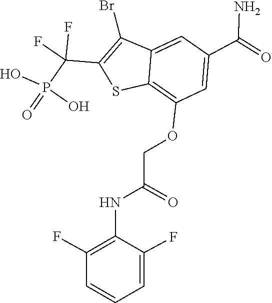 [SMILES: NC(=O)c1cc(OCC(=O)Nc2c(F)cccc2F)c2sc(C(F)(F)P(=O)(O)O)c(Br)c2c1] | 571, 573 |
| 315 | 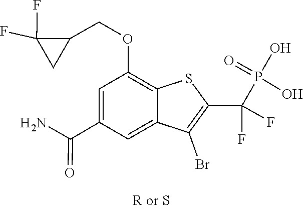 [SMILES: NC(=O)c1cc(OCC2CC2(F)F)c2sc(C(F)(F)P(=O)(O)O)c(Br)c2c1] | 492, 494 |
| 316 | 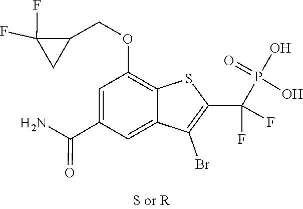 [SMILES: NC(=O)c1cc(OCC2CC2(F)F)c2sc(C(F)(F)P(=O)(O)O)c(Br)c2c1] | 492, 494 |
| 317 | 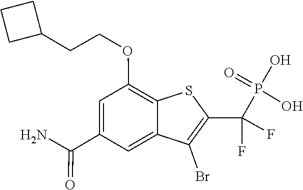 [SMILES: NC(=O)c1cc(OCCC2CCC2)c2sc(C(F)(F)P(=O)(O)O)c(Br)c2c1] | 484, 486 |
| 318 | 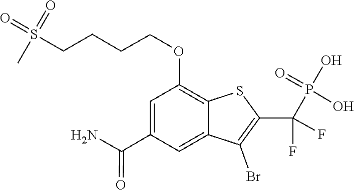 [SMILES: CS(=O)(=O)CCCCOc1cc(C(N)=O)cc2c(Br)c(C(F)(F)P(=O)(O)O)sc12] | 536, 538 |
| 319 | 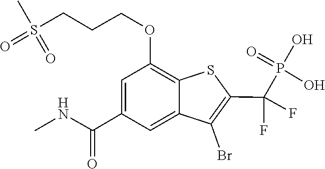 [SMILES: CNC(=O)c1cc(OCCCS(C)(=O)=O)c2sc(C(F)(F)P(=O)(O)O)c(Br)c2c1] | 536, 538 |
| 320 | 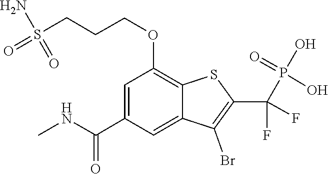 [SMILES: CNC(=O)c1cc(OCCCS(N)(=O)=O)c2sc(C(F)(F)P(=O)(O)O)c(Br)c2c1] | 537, 539 |
| 321 | 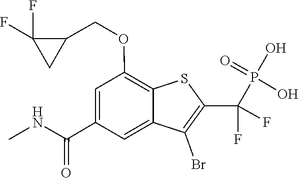 [SMILES: CNC(=O)c1cc(OCC2CC2(F)F)c2sc(C(F)(F)P(=O)(O)O)c(Br)c2c1] | 506, 508 |
| 322 | 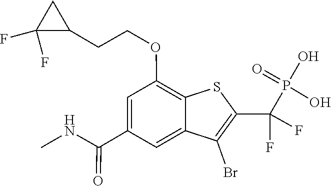 [SMILES: CNC(=O)c1cc(OCCC2CC2(F)F)c2sc(C(F)(F)P(=O)(O)O)c(Br)c2c1] | 520, 522 |
| 323 | 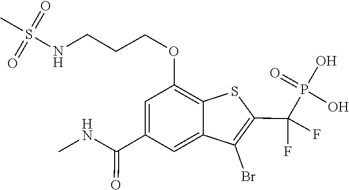 [SMILES: CNC(=O)c1cc(OCCCNS(C)(=O)=O)c2sc(C(F)(F)P(=O)(O)O)c(Br)c2c1] | 551, 553 |
| 324 | 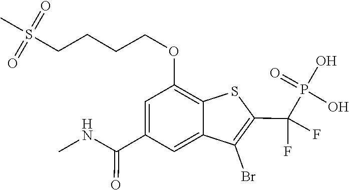 [SMILES: CNC(=O)c1cc(OCCCCS(C)(=O)=O)c2sc(C(F)(F)P(=O)(O)O)c(Br)c2c1] | 550, 552 |
| 325 | 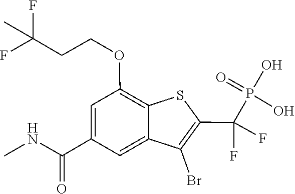 [SMILES: CNC(=O)c1cc(OCCC(C)(F)F)c2sc(C(F)(F)P(=O)(O)O)c(Br)c2c1] | 508, 510 |
| 326 | 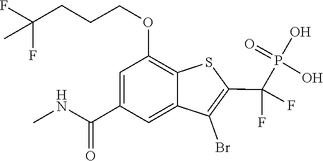 [SMILES: CNC(=O)c1cc(OCCCC(C)(F)F)c2sc(C(F)(F)P(=O)(O)O)c(Br)c2c1] | 522, 524 |
| 327 | 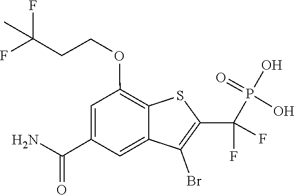 [SMILES: CC(F)(F)CCOc1cc(C(N)=O)cc2c(Br)c(C(F)(F)P(=O)(O)O)sc12] | 494, 496 |
| 328 | 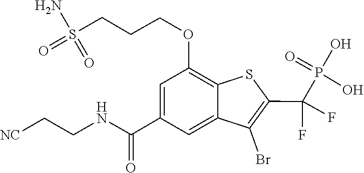 [SMILES: N#CCCNC(=O)c1cc(OCCCS(N)(=O)=O)c2sc(C(F)(F)P(=O)(O)O)c(Br)c2c1] | 574, 576 [M − H]⁻ |
| 329 | 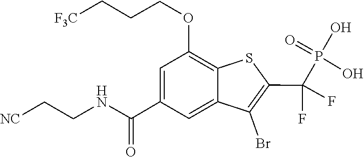 [SMILES: N#CCCNC(=O)c1cc(OCCCC(F)(F)F)c2sc(C(F)(F)P(=O)(O)O)c(Br)c2c1] | 563, 565 [M − H]⁻ |
| 330 | 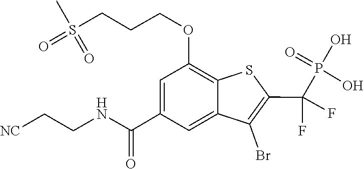 [SMILES: CS(=O)(=O)CCCOc1cc(C(=O)NCCC#N)cc2c(Br)c(C(F)(F)P(=O)(O)O)sc12] | 573, 575 [M − H]⁻ |
| 331 | 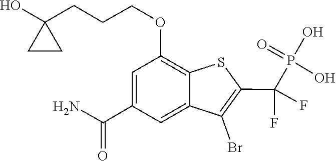 [SMILES: NC(=O)c1cc(OCCCC2(O)CC2)c2sc(C(F)(F)P(=O)(O)O)c(Br)c2c1] | 500, 502 |
| 332 | 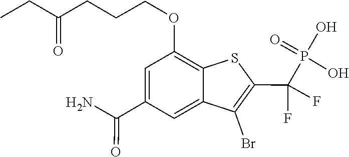 [SMILES: CCC(=O)CCCOc1cc(C(N)=O)cc2c(Br)c(C(F)(F)P(=O)(O)O)sc12] | 500, 502 |
| 333 | 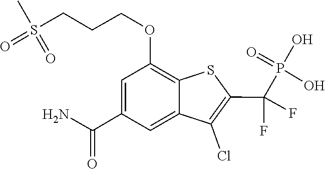 [SMILES: CS(=O)(=O)CCCOc1cc(C(N)=O)cc2c(Cl)c(C(F)(F)P(=O)(O)O)sc12] | 478 |
| 334 | 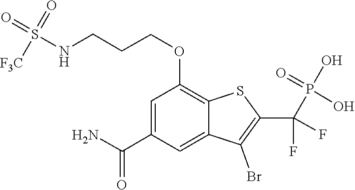 [SMILES: NC(=O)c1cc(OCCCNS(=O)(=O)C(F)(F)F)c2sc(C(F)(F)P(=O)(O)O)c(Br)c2c1] | 591, 593 |
| 335 | 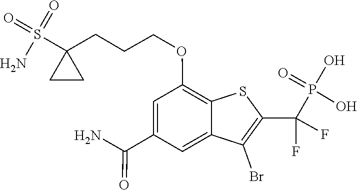 [SMILES: NC(=O)c1cc(OCCCC2(S(N)(=O)=O)CC2)c2sc(C(F)(F)P(=O)(O)O)c(Br)c2c1] | 563, 565 |
| 336 | 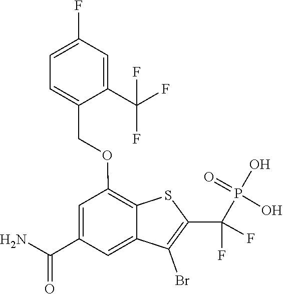 [SMILES: NC(=O)c1cc(OCc2ccc(F)cc2C(F)(F)F)c2sc(C(F)(F)P(=O)(O)O)c(Br)c2c1] | 576, 578 [M − H]⁻ |
| 337 | 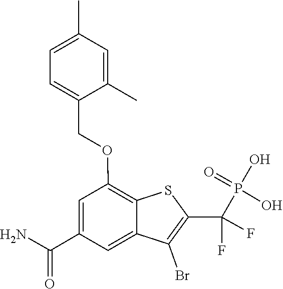 [SMILES: Cc1ccc(COc2cc(C(N)=O)cc3c(Br)c(C(F)(F)P(=O)(O)O)sc23)c(C)c1] | 520, 522 |
| 338 | 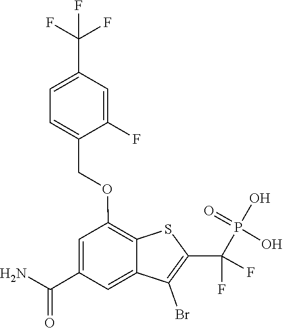 [SMILES: NC(=O)c1cc(OCc2ccc(C(F)(F)F)cc2F)c2sc(C(F)(F)P(=O)(O)O)c(Br)c2c1] | 576, 578 [M − H]⁻ |
| 339 | 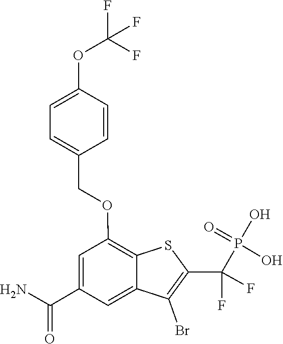 [SMILES: NC(=O)c1cc(OCc2ccc(OC(F)(F)F)cc2)c2sc(C(F)(F)P(=O)(O)O)c(Br)c2c1] | 576, 578 |
| 340 | 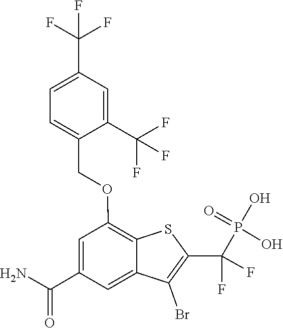 [SMILES: NC(=O)c1cc(OCc2ccc(C(F)(F)F)cc2C(F)(F)F)c2sc(C(F)(F)P(=O)(O)O)c(Br)c2c1] | 626, 628 [M − H]⁻ |
| 341 | 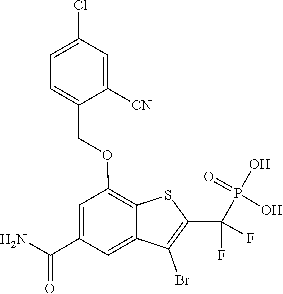 [SMILES: N#Cc1cc(Cl)ccc1COc1cc(C(N)=O)cc2c(Br)c(C(F)(F)P(=O)(O)O)sc12] | 549, 551 [M − H]⁻ |
| 342 | 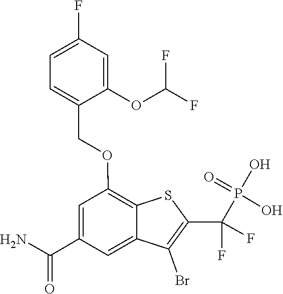 [SMILES: NC(=O)c1cc(OCc2ccc(F)cc2OC(F)F)c2sc(C(F)(F)P(=O)(O)O)c(Br)c2c1] | 576, 578 |
| 343 | 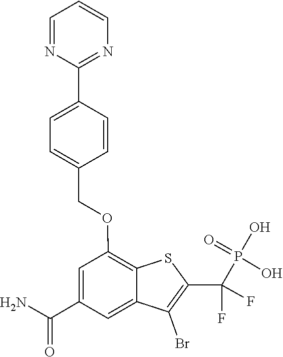 [SMILES: NC(=O)c1cc(OCc2ccc(-c3ncccn3)cc2)c2sc(C(F)(F)P(=O)(O)O)c(Br)c2c1] | 568, 570 [M − H]⁻ |
| 344 | 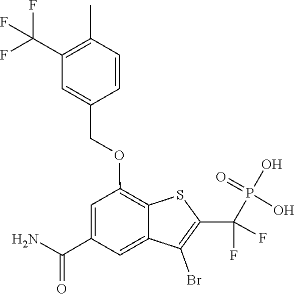 [SMILES: Cc1ccc(COc2cc(C(N)=O)cc3c(Br)c(C(F)(F)P(=O)(O)O)sc23)cc1C(F)(F)F] | 574, 576 |
| 345 | 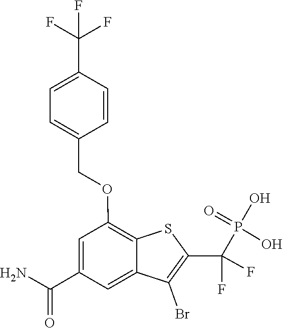 [SMILES: NC(=O)c1cc(OCc2ccc(C(F)(F)F)cc2)c2sc(C(F)(F)P(=O)(O)O)c(Br)c2c1] | 560, 562 |
| 346 | 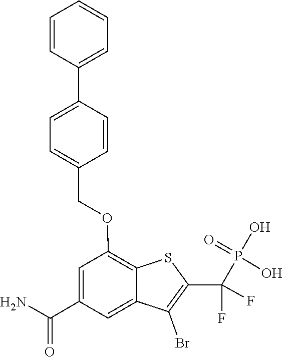 [SMILES: NC(=O)c1cc(OCc2ccc(-c3ccccc3)cc2)c2sc(C(F)(F)P(=O)(O)O)c(Br)c2c1] | 568, 570 |
| 347 | 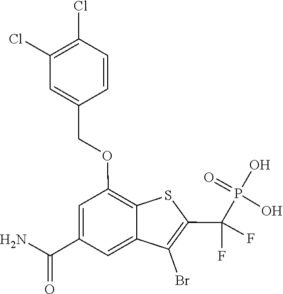 [SMILES: NC(=O)c1cc(OCc2ccc(Cl)c(Cl)c2)c2sc(C(F)(F)P(=O)(O)O)c(Br)c2c1] | 560, 562 |
| 348 | 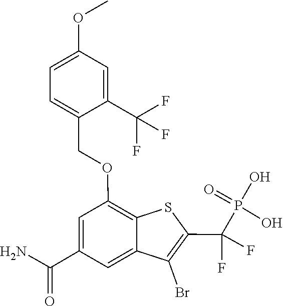 [SMILES: COc1ccc(COc2cc(C(N)=O)cc3c(Br)c(C(F)(F)P(=O)(O)O)sc23)c(C(F)(F)F)c1] | 588, 590 [M − H]⁻ |
| 349 | 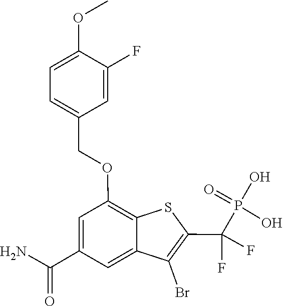 [SMILES: COc1ccc(COc2cc(C(N)=O)cc3c(Br)c(C(F)(F)P(=O)(O)O)sc23)cc1F] | 538, 540 [M − H]⁻ |
| 350 | 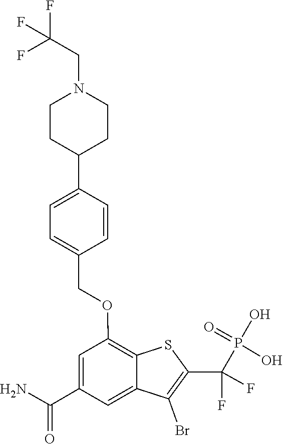 [SMILES: NC(=O)c1cc(OCc2ccc(C3CCN(CC(F)(F)F)CC3)cc2)c2sc(C(F)(F)P(=O)(O)O)c(Br)c2c1] | 655, 657 [M − H]⁻ |
| 351 | 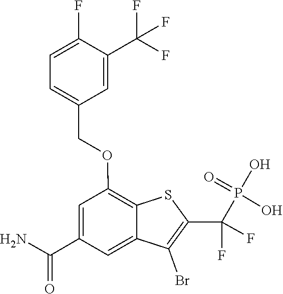 [SMILES: NC(=O)c1cc(OCc2ccc(F)c(C(F)(F)F)c2)c2sc(C(F)(F)P(=O)(O)O)c(Br)c2c1] | 578, 580 |
| 352 | 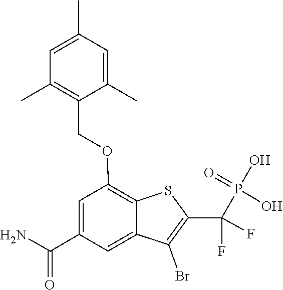 [SMILES: Cc1cc(C)c(COc2cc(C(N)=O)cc3c(Br)c(C(F)(F)P(=O)(O)O)sc23)c(C)c1] | 534, 536 |
| 353 | 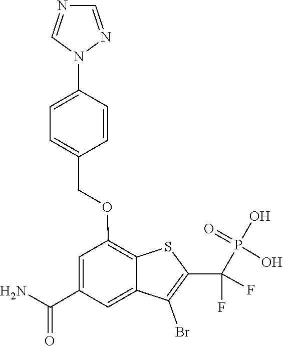 [SMILES: NC(=O)c1cc(OCc2ccc(-n3cncn3)cc2)c2sc(C(F)(F)P(=O)(O)O)c(Br)c2c1] | 557, 559 [M − H]⁻ |
| 354 | 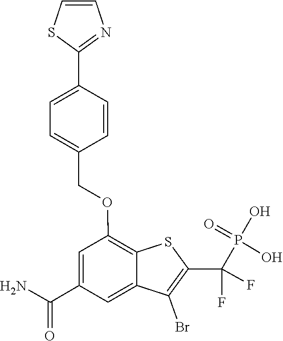 [SMILES: NC(=O)c1cc(OCc2ccc(-c3nccs3)cc2)c2sc(C(F)(F)P(=O)(O)O)c(Br)c2c1] | 573, 575 [M − H]⁻ |
| 355 | 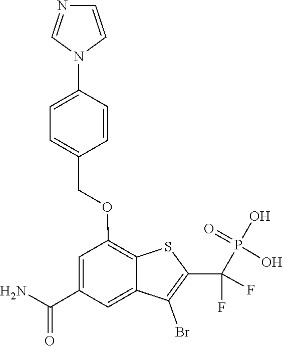 [SMILES: NC(=O)c1cc(OCc2ccc(-n3ccnc3)cc2)c2sc(C(F)(F)P(=O)(O)O)c(Br)c2c1] | 556, 558 [M − H]⁻ |
| 356 | 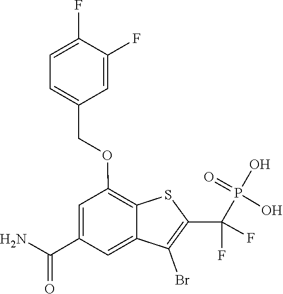 [SMILES: NC(=O)c1cc(OCc2ccc(F)c(F)c2)c2sc(C(F)(F)P(=O)(O)O)c(Br)c2c1] | 528, 530 |
| 357 | 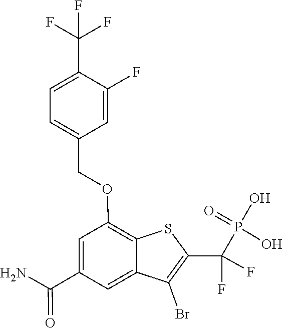 [SMILES: NC(=O)c1cc(OCc2ccc(C(F)(F)F)c(F)c2)c2sc(C(F)(F)P(=O)(O)O)c(Br)c2c1] | 578, 580 |
| 358 | 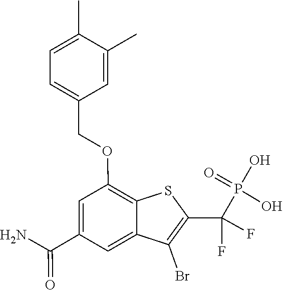 [SMILES: Cc1ccc(COc2cc(C(N)=O)cc3c(Br)c(C(F)(F)P(=O)(O)O)sc23)cc1C] | 518, 510 [M − H]⁻ |
| 359 | 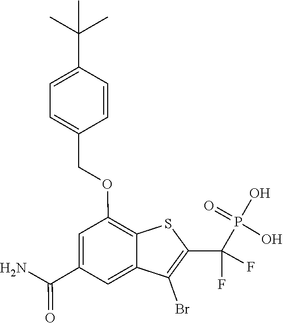 [SMILES: CC(C)(C)c1ccc(COc2cc(C(N)=O)cc3c(Br)c(C(F)(F)P(=O)(O)O)sc23)cc1] | 548, 550 |
| 360 | 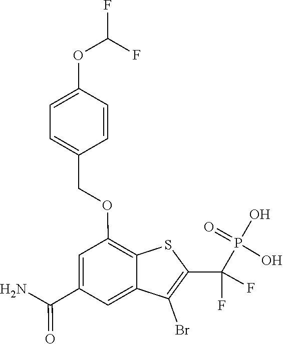 [SMILES: NC(=O)c1cc(OCc2ccc(OC(F)F)cc2)c2sc(C(F)(F)P(=O)(O)O)c(Br)c2c1] | 558, 560 |
| 361 | 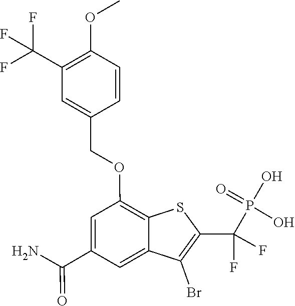 [SMILES: COc1ccc(COc2cc(C(N)=O)cc3c(Br)c(C(F)(F)P(=O)(O)O)sc23)cc1C(F)(F)F] | 588, 590 [M − H]⁻ |
| 362 | 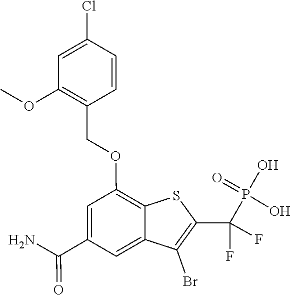 [SMILES: COc1cc(Cl)ccc1COc1cc(C(N)=O)cc2c(Br)c(C(F)(F)P(=O)(O)O)sc12] | 554, 556 [M − H]⁻ |
| 363 | 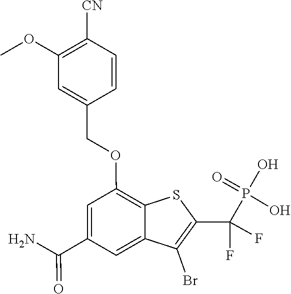 [SMILES: COc1cc(COc2cc(C(N)=O)cc3c(Br)c(C(F)(F)P(=O)(O)O)sc23)ccc1C#N] | 545, 547 [M − H]⁻ |

^{a}Products were separated into pure stereoisomers prior to TMS-Br deprotection using the following conditions: ChiralPak AS-H; 21 × 250 mm, 5 um; 20% methanol in CO₂ with 0.1% ammonium hydroxide; flow rate = 70 mL/min

### Example 27

### Preparation of Compound 258

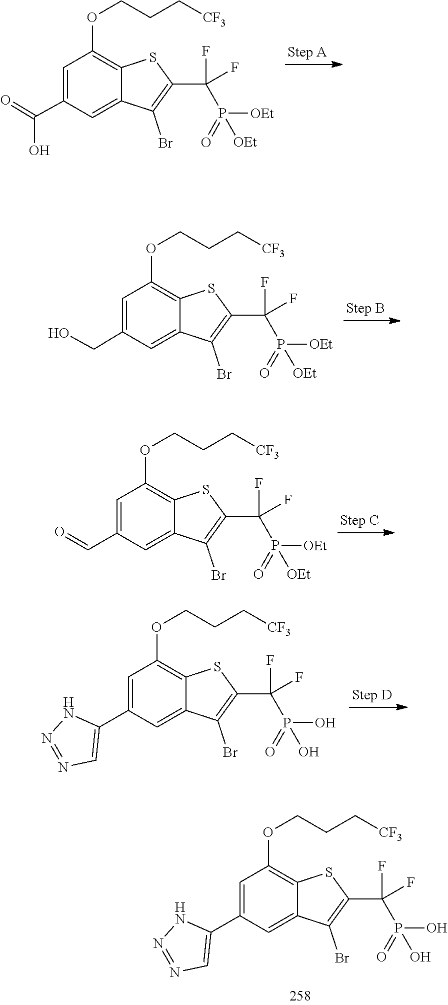

### Step A: Synthesis of diethyl ((3-bromo-5-(hydroxymethyl)-7-(4,4,4-trifluorobutoxy)benzo[b]thiophen-2-yl)difluoromethyl)phosphonate

To a mixture of compound Int-8a (200 mg, 0.35 mmol) in THF (3 mL) was added dropwise borane-THF (1M in THF) (3.2 mL, 3.2 mmol) at 0° C. under argon. The resulting solution was allowed to stir for 16 hours at room temperature. The reaction was quenched with MeOH (10 mL), and cool water (50 mL), and extracted with EtOAc (3×100 mL). The combined organic extracts were washed with brine (100 mL), dried over anhydrous Na₂SO₄ and concentrated in vacuo. The resulting residue was purified using silica gel chromatography (eluting ethyl acetate in petroleum ether) to provide diethyl ((3-bromo-5-(hydroxymethyl)-7-(4,4,4-trifluorobutoxy)benzo[b]thiophen-2-yl)difluoromethyl)phosphonate. MS (ESI, m/z): 555, 557 (M+H)⁺

### Step B: Synthesis of diethyl ((3-bromo-5-formyl-7-(4,4,4-trifluorobutoxy)benzo[b]thiophen-2-yl)difluoromethyl)phosphonate

To a mixture of diethyl ((3-bromo-5-(hydroxymethyl)-7-(4,4,4-trifluorobutoxy)benzo[b]thiophen-2-yl)difluoromethyl)phosphonate (140 mg, 0.25 mmol) in DCM (2 mL) was added Dess-Martin periodinane (210 mg, 0.5 mmol) at 0° C. under argon. The resulting mixture was allowed to stir for 1 hour at room temperature. The reaction was quenched with saturated aqueous sodium bicarbonate (10 mL), and extracted with EtOAc (3×50 mL). The combined organic extracts were washed with brine (60 mL), dried over anhydrous Na₂SO₄ and concentrated in vacuo. The resulting residue was purified using silica gel chromatography (eluting ethyl acetate in petroleum ether) to provide diethyl ((3-bromo-5-formyl-7-(4,4,4-trifluorobutoxy)benzo[b]thiophen-2-yl)difluoromethyl)phosphonate. MS (ESI, m/z): 553, 555 (M+H)⁺

### Step C: Synthesis of ethyl hydrogen ((3-bromo-5-(1H-1,2,3-triazol-5-yl)-7-(4,4,4-trifluorobutoxy)benzo[b]thiophen-2-yl)difluoromethyl)phosphonate

To a mixture of diethyl ((3-bromo-5-formyl-7-(4,4,4-trifluorobutoxy)benzo[b]thiophen-2-yl)difluoromethyl)phosphonate (30 mg, 0.054 mmol) in DMF (0.3 mL) was added acetic acid (0.025 mL, 0.43 mmol), nitromethane (0.0059 mL, 0.11 mmol), sodium azide (7.1 mg, 0.11 mmol), and ammonium acetate (4.2 mg, 0.054 mmol) under argon. The resulting mixture was allowed to stir for 1.5 hours at 140° C. The reaction was cooled to room temperature, and directly purified using reverse phase HPLC (eluting acetonitrile in water with NH₄HCO₃ modifier) to provide ethyl hydrogen ((3-bromo-5-(1H-1,2,3-triazol-5-yl)-7-(4,4,4-trifluorobutoxy)benzo[b]thiophen-2-yl)difluoromethyl)phosphonate. MS (ESI, m/z): 562, 564 (M−H)⁻

### Step D: Synthesis of ((3-bromo-5-(1H-1,2,3-triazol-5-yl)-7-(4,4,4-trifluorobutoxy)benzo[b]thiophen-2-yl)difluoromethyl)phosphonic acid

To a solution of ethyl hydrogen ((3-bromo-5-(1H-1,2,3-triazol-5-yl)-7-(4,4,4-trifluorobutoxy)benzo[b]thiophen-2-yl)difluoromethyl)phosphonate (6.6 mg, 0.011 mmol) in DMF (0.12 mL) was added TMS-Br (0.044 mL, 0.34 mmol) under argon. The resulting solution was allowed to stir for 1 hour at 60° C. The reaction was cooled to room temperature, quenched by addition of EtOH (0.1 mL), and concentrated in vacuo. The resulting residue was purified using reverse phase HPLC (eluting acetonitrile in water with NH₄HCO₃ modifier) to provide ((3-bromo-5-(1H-1,2,3-triazol-5-yl)-7-(4,4,4-trifluorobutoxy)benzo[b]thiophen-2-yl)difluoromethyl)phosphonic acid (compound 258). ¹H NMR (400 MHz, deuterium oxide) δ 7.70 (s, 1H), 7.25 (s, 1H), 6.74 (s, 1H), 4.01 (t, J=6.0 Hz, 2H), 2.38-2.22 (m, 2H), 2.00-1.92 (m, 2H). MS (ESI, m/z): 536, 538 (M+H)⁺

The following illustrative compounds were made using the methods described in the Example immediately above, and substituting the appropriate reactants and/or reagents.

| | | Mass |
|---|---|---|
| Compound | Structure | [M + H]⁺ |
| 364 | 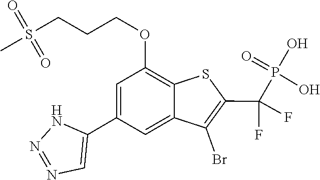 [SMILES: CS(=O)(=O)CCCOc1cc(-c2cnn[nH]2)cc2c(Br)c(C(F)(F)P(=O)(O)O)sc12] | 546, 548 |
| 365 | 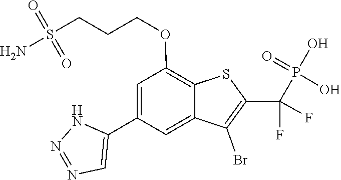 [SMILES: NS(=O)(=O)CCCOc1cc(-c2cnn[nH]2)cc2c(Br)c(C(F)(F)P(=O)(O)O)sc12] | 547, 549 |
| 366 | 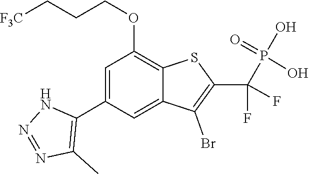 [SMILES: Cc1nn[nH]c1-c1cc(OCCCC(F)(F)F)c2sc(C(F)(F)P(=O)(O)O)c(Br)c2c1] | 550, 552 |
| 367 | 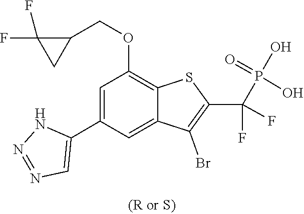 [SMILES: O=P(O)(O)C(F)(F)c1sc2c(OCC3CC3(F)F)cc(-c3cnn[nH]3)cc2c1Br] | 516, 518 |
| 368 | 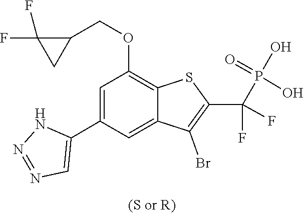 [SMILES: O=P(O)(O)C(F)(F)c1sc2c(OCC3CC3(F)F)cc(-c3cnn[nH]3)cc2c1Br] | 516, 518 |
| 369 | 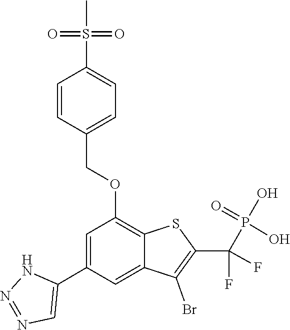 [SMILES: CS(=O)(=O)c1ccc(COc2cc(-c3cnn[nH]3)cc3c(Br)c(C(F)(F)P(=O)(O)O)sc23)cc1] | 594, 596 |

^{a}Products were separated into pure stereoisomers following Step B using the following conditions: ChiralPak AS-3; 4.6 × 100 mm, 3 um; 20% isopropanol in hexane with 0.1% diethylamine; flow rate = 1 mL/min. The separated stereoisomers were then subjected to Steps C and D.

### Example 28

### Preparation of Compound 259

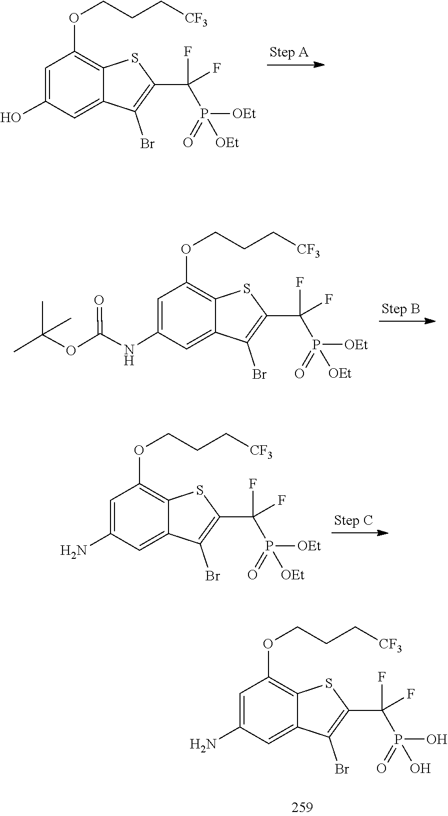 [SMILES: CCOP(=O)(OCC)C(F)(F)c1sc2c(OCCCC(F)(F)F)cc(O)cc2c1Br ; CCOP(=O)(OCC)C(F)(F)c1sc2c(OCCCC(F)(F)F)cc(NC(=O)OC(C)(C)C)cc2c1Br ; CCOP(=O)(OCC)C(F)(F)c1sc2c(OCCCC(F)(F)F)cc(N)cc2c1Br ; Nc1cc(OCCCC(F)(F)F)c2sc(C(F)(F)P(=O)(O)O)c(Br)c2c1]

### Step A: Synthesis of tert-butyl (3-bromo-2-((diethoxyphosphoryl)difluoromethyl)-7-(4,4,4-trifluorobutoxy)benzo[b]thiophen-5-yl)carbamate

To a mixture of 3-bromo-2-((diethoxyphosphoryl)difluoromethyl)-7-(4,4,4-trifluorobutoxy)benzo[b]thiophene-5-carboxylic acid (1000 mg, 1.8 mmol), and triethylamine (0.27 mL, 1.9 mmol) in toluene (10 mL), and t-BuOH (10 mL) was added diphenylphosphoryl azide (0.38 mL, 1.8 mmol) under argon. The mixture was allowed to stir for 16 hours at 80° C. The mixture was cooled to room temperature, diluted with EtOAc (100 mL), and washed with brine (2×100 mL). The organic layer was dried over anhydrous Na₂SO₄ and concentrated in vacuo. The resulting residue was purified using silica gel chromatography (eluting ethyl acetate in petroleum ether) to provide tert-butyl (3-bromo-2-((diethoxyphosphoryl)difluoromethyl)-7-(4,4,4-trifluorobutoxy)benzo[b]thiophen-5-yl)carbamate. MS (ESI, m/z): 640, 642 (M+H)⁺

### Step B: Synthesis of diethyl ((5-amino-3-bromo-7-(4,4,4-trifluorobutoxy)benzo[b]thiophen-2-yl)difluoromethyl)phosphonate

To a mixture of tert-butyl (3-bromo-2-((diethoxyphosphoryl)difluoromethyl)-7-(4,4,4-trifluorobutoxy)benzo[b]thiophen-5-yl)carbamate (340 mg, 0.53 mmol) in DCM (6 mL) was added TFA (2 mL, 26 mmol). The mixture was allowed to stir for 30 minutes at room temperature then concentrated in vacuo. The resulting residue was taken up in EtOAc (10 mL), and the pH was adjusted to 8 with saturated aqueous sodium bicarbonate. The aqueous layer was extracted with EtOAc (2×50 mL). The combined organic extracts were washed with brine (100 mL), dried over anhydrous Na₂SO₄ and concentrated in vacuo. The resulting residue was purified using silica gel chromatography (eluting ethyl acetate in petroleum ether) to provide diethyl ((5-amino-3-bromo-7-(4,4,4-trifluorobutoxy)benzo[b]thiophen-2-yl)difluoromethyl)phosphonate. MS (ESI, m/z): 540, 542 (M+H)⁺

### Step C: Synthesis of ((5-amino-3-bromo-7-(4,4,4-trifluorobutoxy)benzo[b]thiophen-2-yl)difluoromethyl)phosphonic acid

To a solution of diethyl ((5-amino-3-bromo-7-(4,4,4-trifluorobutoxy)benzo[b]thiophen-2-yl)difluoromethyl)phosphonate (25 mg, 0.046 mmol) in DMF (0.36 mL) was added TMS-Br (0.18 mL, 1.4 mmol) under argon. The resulting solution was allowed to stir for 1.5 hours at 60° C. The reaction was cooled to room temperature, quenched by addition of EtOH (0.5 mL), and concentrated in vacuo. The resulting residue was purified using reverse phase HPLC (eluting acetonitrile in water with NH₄HCO₃ modifier) to provide ((5-amino-3-bromo-7-(4,4,4-trifluorobutoxy)benzo[b]thiophen-2-yl)difluoromethyl)phosphonic acid (compound 259). ¹H NMR (400 MHz, DMSO-d₆) δ 7.26 (s, 2H), 6.59 (s, 1H), 6.44 (s, 1H), 4.15 (t, J=6.1 Hz, 2H), 2.47-2.38 (m, 2H), 2.05-1.97 (m, 2H). MS (ESI, m/z): 484, 486 (M+H)⁺

### Example 29

### Preparation of Compound 260

### Preparation of ((3-bromo-5-(1H-pyrazol-5-yl)-7-(4,4,4-trifluorobutoxy)benzo[b]thiophen-2-yl)difluoromethyl)phosphonic acid

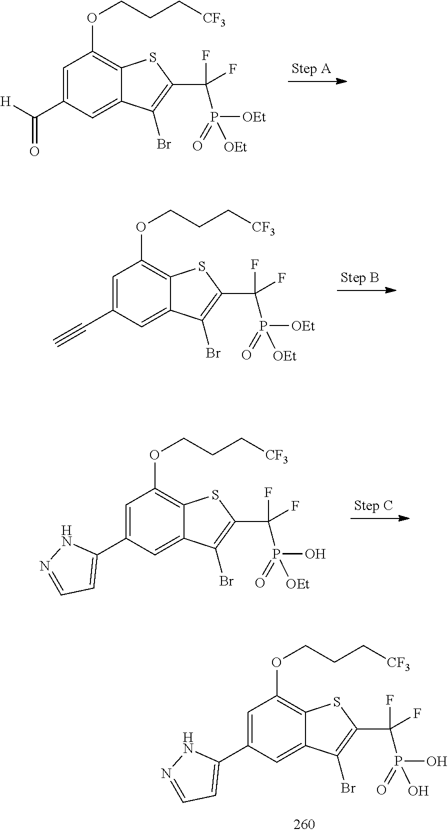 [SMILES: CCOP(=O)(OCC)C(F)(F)c1sc2c(OCCCC(F)(F)F)cc(C=O)cc2c1Br ; C#Cc1cc(OCCCC(F)(F)F)c2sc(C(F)(F)P(=O)(OCC)OCC)c(Br)c2c1 ; CCOP(=O)(O)C(F)(F)c1sc2c(OCCCC(F)(F)F)cc(-c3ccn[nH]3)cc2c1Br ; O=P(O)(O)C(F)(F)c1sc2c(OCCCC(F)(F)F)cc(-c3ccn[nH]3)cc2c1Br]

### Step A: Synthesis of diethyl ((3-bromo-5-ethynyl-7-(4,4,4-trifluorobutoxy)benzo[b]thiophen-2-yl)difluoromethyl)phosphonate

To a mixture of diethyl ((3-bromo-5-formyl-7-(4,4,4-trifluorobutoxy)benzo[b]thiophen-2-yl)difluoromethyl)phosphonate (100 mg, 0.18 mmol), and potassium carbonate (38 mg, 0.27 mmol) in MeOH (1 mL) was added dimethyl (1-diazo-2-oxopropyl)phosphonate (52 mg, 0.27 mmol) under argon. The mixture was allowed to stir for 1 hour at room temperature. The reaction was diluted with water (20 mL), and extracted with EtOAc (50 mL). The organic layer was dried over anhydrous Na₂SO₄ and concentrated in vacuo. The resulting residue was purified using Prep-TLC (developed by ethyl acetate/petroleum ether) to provide diethyl ((3-bromo-5-ethynyl-7-(4,4,4-trifluorobutoxy)benzo[b]thiophen-2-yl)difluoromethyl)phosphonate. MS (ESI, m/z): 549, 551 (M+H)⁺

### Step B: Synthesis of ethyl hydrogen ((3-bromo-5-(1H-pyrazol-5-yl)-7-(4,4,4-trifluorobutoxy)benzo[b]thiophen-2-yl)difluoromethyl)phosphonate

To a mixture of diethyl ((3-bromo-5-ethynyl-7-(4,4,4-trifluorobutoxy)benzo[b]thiophen-2-yl)difluoromethyl)phosphonate (70 mg, 0.13 mmol), N-isocyano-1,1,1-triphenyl-15-phosphanimine (77 mg, 0.26 mmol), lithium methanolate (15 mg, 0.38 mmol), Mo(CO)₆ (1.7 mg, 0.0064 mmol), and silver carbonate (18 mg, 0.064 mmol) in THF (0.4 mL) was added water (0.04 mL) dropwise under argon. The resulting solution was allowed to stir for 16 hours at 60° C. The reaction mixture was cooled to room temperature, and diluted with water (20 mL), and extracted with EtOAc (20 mL). The organic layer was washed with brine (20 mL), dried over anhydrous Na₂SO₄ and concentrated in vacuo. The resulting residue was purified using reverse phase HPLC (eluting acetonitrile in water) to provide ethyl hydrogen ((3-bromo-5-(1H-pyrazol-5-yl)-7-(4,4,4-trifluorobutoxy)benzo[b]thiophen-2-yl)difluoromethyl)phosphonate. MS (ESI, m/z): 563, 565 (M+H)⁺

### Step C: Synthesis of ((3-bromo-5-(1H-pyrazol-5-yl)-7-(4,4,4-trifluorobutoxy)benzo[b]thiophen-2-yl)difluoromethyl)phosphonic acid

To a solution of ethyl hydrogen ((3-bromo-5-(1H-pyrazol-5-yl)-7-(4,4,4-trifluorobutoxy)benzo[b]thiophen-2-yl)difluoromethyl)phosphonate (37 mg, 0.066 mmol) in DMF (0.51 mL) was added TMS-Br (0.26 mL, 2.0 mmol) at 0° C. under argon. The resulting solution was allowed to stir for 2 hours at 40° C. The reaction was cooled to room temperature, quenched by addition of EtOH (0.2 mL), and concentrated in vacuo. The resulting residue was purified using reverse phase HPLC (eluting acetonitrile in water with NH₄HCO₃ modifier) to provide ((3-bromo-5-(1H-pyrazol-5-yl)-7-(4,4,4-trifluorobutoxy)benzo[b]thiophen-2-yl)difluoromethyl)phosphonic acid (compound 260). ¹H NMR (400 MHz, deuterium oxide) δ 7.60 (s, 1H), 7.53 (br s, 1H), 7.02 (br s, 1H), 6.55 (s, 1H), 4.16-4.08 (m, 2H), 2.43-2.28 (m, 2H), 2.08-1.97 (m, 2H). MS (ESI, m/z): 535, 537 (M+H)⁺

### Example 30

### Preparation of Compound 261

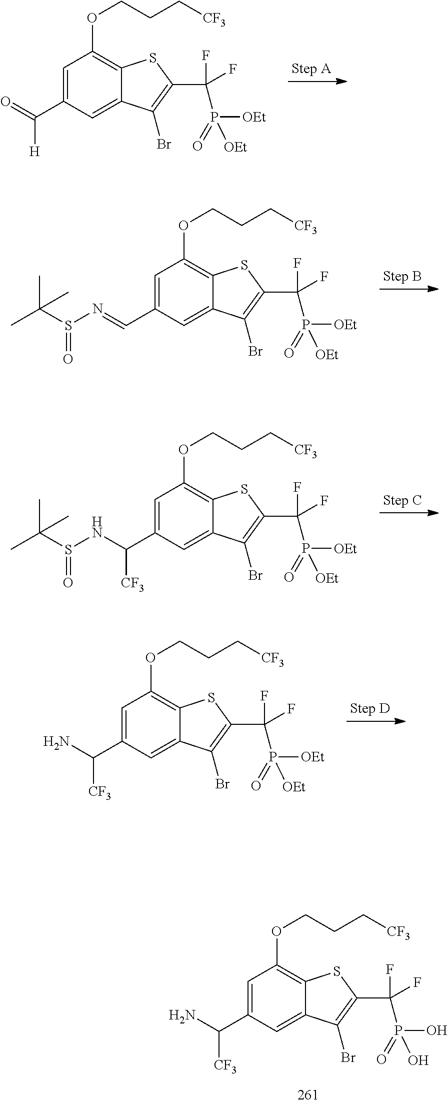 [SMILES: CCOP(=O)(OCC)C(F)(F)c1sc2c(OCCCC(F)(F)F)cc(C=O)cc2c1Br ; CCOP(=O)(OCC)C(F)(F)c1sc2c(OCCCC(F)(F)F)cc(/C=N/S(=O)C(C)(C)C)cc2c1Br ; CCOP(=O)(OCC)C(F)(F)c1sc2c(OCCCC(F)(F)F)cc(C(NS(=O)C(C)(C)C)C(F)(F)F)cc2c1Br ; CCOP(=O)(OCC)C(F)(F)c1sc2c(OCCCC(F)(F)F)cc(C(N)C(F)(F)F)cc2c1Br ; NC(c1cc(OCCCC(F)(F)F)c2sc(C(F)(F)P(=O)(O)O)c(Br)c2c1)C(F)(F)F]

### Step A: Synthesis of diethyl (E)-((3-bromo-5-(((tert-butylsulfinyl)imino)methyl)-7-(4,4,4-trifluorobutoxy)benzo[b]thiophen-2-yl)difluoromethyl)phosphonate

To a mixture of diethyl ((3-bromo-5-formyl-7-(4,4,4-trifluorobutoxy)benzo[b]thiophen-2-yl)difluoromethyl)phosphonate (180 mg, 0.33 mmol), and tetraethoxytitanium (220 mg, 0.98 mmol) in THF (2 mL) was added 2-methylpropane-2-sulfinamide (79 mg, 0.65 mmol) at room temperature. The resulting solution was allowed to stir for 16 hours at 75° C. The reaction was cooled to room temperature, and was directly purified using silica gel chromatography (eluting ethyl acetate in petroleum ether) to provide diethyl (E)-((3-bromo-5-(((tert-butylsulfinyl)imino)methyl)-7-(4,4,4-trifluorobutoxy)benzo[b]thiophen-2-yl)difluoromethyl)phosphonate. MS (ESI, m/z): 656, 658 (M+H)⁺

### Step B: Synthesis of diethyl ((3-bromo-5-(1-((tert-butylsulfinyl)amino)-2,2,2-trifluoroethyl)-7-(4,4,4-trifluorobutoxy)benzo[b]thiophen-2-yl)difluoromethyl)phosphonate

To a solution of diethyl (E)-((3-bromo-5-(((tert-butylsulfinyl)imino)methyl)-7-(4,4,4-trifluorobutoxy)benzo[b]thiophen-2-yl)difluoromethyl)phosphonate (170 mg, 0.26 mmol), and tetrabutylammonium difluorotriphenylsilicate (IV) (154 mg, 0.29 mmol) in THF (2.6 mL) was added trimethyl(trifluoromethyl)silane (110 mg, 0.78 mmol) at −55° C. under argon. The resulting solution was allowed to stir for 1 hour at −55° C. then saturated aqueous NH₄Cl (0.6 mL) was added and the reaction was allowed to warm to room temperature. The reaction was diluted with water (30 mL), and extracted with EtOAc (2×35 mL). The combined organic extracts were washed with brine (50 mL), dried over anhydrous Na₂SO₄ and concentrated in vacuo. The resulting residue was purified using silica gel chromatography (eluting ethyl acetate in petroleum ether) to provide diethyl ((3-bromo-5-(1-((tert-butylsulfinyl)amino)-2,2,2-trifluoroethyl)-7-(4,4,4-trifluorobutoxy)benzo[b]thiophen-2-yl)difluoromethyl)phosphonate. MS (ESI, m/z): 724, 726 (M−H)⁻

### Step C: Synthesis of diethyl (R or S)-((5-(1-amino-2,2,2-trifluoroethyl)-3-bromo-7-(4,4,4-trifluorobutoxy)benzo[b]thiophen-2-yl)difluoromethyl)phosphonate

To a mixture of diethyl ((3-bromo-5-(1-((tert-butylsulfinyl)amino)-2,2,2-trifluoroethyl)-7-(4,4,4-trifluorobutoxy)benzo[b]thiophen-2-yl)difluoromethyl)phosphonate (140 mg, 0.19 mmol) in MeOH (1.5 mL) was added HCl (4M in 1,4-dioxane) (0.096 mL, 0.39 mmol) at room temperature under argon. The resulting solution was allowed to stir for 1 hour at room temperature. The reaction mixture was concentrated in vacuo, reconstituted with MeOH (1 mL), and pH adjusted between 7-8 with aqueous sodium bicarbonate. The resulting solution was purified using reverse phase HPLC (eluting with acetonitrile in water) to provide diethyl ((5-(1-amino-2,2,2-trifluoroethyl)-3-bromo-7-(4,4,4-trifluorobutoxy)benzo[b]thiophen-2-yl)difluoromethyl)phosphonate as a white solid. Solid was further purified using chiral HPLC (CHIRALPAK IE-3 column, 20% EtOH in petroleum ether with 0.1% diethylamine) to provide diethyl (R or S)-((5-(1-amino-2,2,2-trifluoroethyl)-3-bromo-7-(4,4,4-trifluorobutoxy)benzo[b]thiophen-2-yl)difluoromethyl)phosphonate as the first eluting peak t_{R}=1.37 minutes. MS (ESI, m/z): 622, 624 (M+H)⁺

### Step D: Synthesis of (R or S)-((5-(1-amino-2,2,2-trifluoroethyl)-3-bromo-7-(4,4,4-trifluorobutoxy)benzo[b]thiophen-2-yl)difluoromethyl)phosphonic acid

To a solution of diethyl (R or S)-((5-(1-amino-2,2,2-trifluoroethyl)-3-bromo-7-(4,4,4-trifluorobutoxy)benzo[b]thiophen-2-yl)difluoromethyl)phosphonate (28 mg, 0.045 mmol) in DMF (0.4 mL) was added TMS-Br (0.12 mL, 0.93 mmol) under argon. The resulting solution was allowed to stir for 1.5 hours at 60° C. The reaction was cooled to room temperature, quenched by addition of EtOH (0.3 mL), and concentrated in vacuo. The resulting residue was purified using reverse phase HPLC (eluting acetonitrile in water with NH₄HCO₃ modifier) to provide (R or S)-((5-(1-amino-2,2,2-trifluoroethyl)-3-bromo-7-(4,4,4-trifluorobutoxy)benzo[b]thiophen-2-yl)difluoromethyl)phosphonic acid (provides separate isomers of compound 261, designated herein as compounds 261a and 261b, with absolute stereochemistry not determined). ¹H NMR (300 MHz, deuterium oxide) δ 7.65 (s, 1H), 7.10 (s, 1H), 4.79-4.71 (m, 1H), 4.32 (t, J=6.2 Hz, 2H), 2.53-2.30 (m, 2H), 2.24-1.99 (m, 2H). MS (ESI, m/z): 564, 566 (M−H)⁻

### Example 31

### Preparation of Compound 262

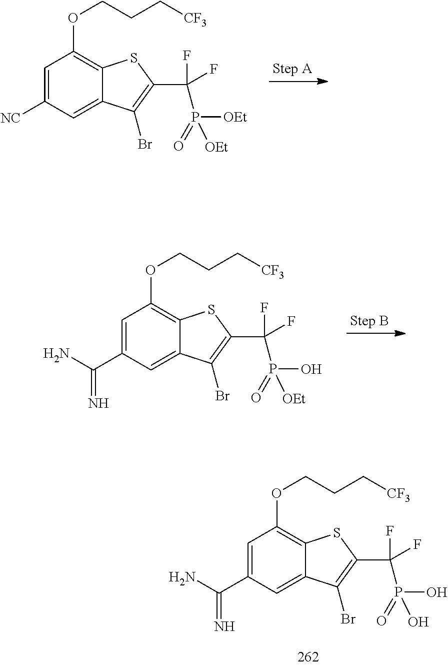 [SMILES: CCOP(=O)(OCC)C(F)(F)c1sc2c(OCCCC(F)(F)F)cc(C#N)cc2c1Br ; CCOP(=O)(O)C(F)(F)c1sc2c(OCCCC(F)(F)F)cc(C(=N)N)cc2c1Br ; N=C(N)c1cc(OCCCC(F)(F)F)c2sc(C(F)(F)P(=O)(O)O)c(Br)c2c1]

### Step A: Synthesis of ethyl hydrogen ((3-bromo-5-carbamimidoyl-7-(4,4,4-trifluorobutoxy)benzo[b]thiophen-2-yl)difluoromethyl)phosphonate

To a mixture of ammonium chloride (200 mg, 3.7 mmol) in toluene (4 mL) was added trimethylaluminum (2 M in toluene, 2 mL, 4 mmol) at 0° C. under argon. The resulting mixture was allowed to stir at room temperature for 2 hours then added to a mixture of diethyl ((3-bromo-5-cyano-7-(4,4,4-trifluorobutoxy)benzo[b]thiophen-2-yl)difluoromethyl)phosphonate (100 mg, 0.18 mmol) in toluene (1 mL) under argon. The resulting mixture was allowed to stir for 16 hours at 80° C. The reaction was cooled to room temperature, and slowly poured into a slurry of silica gel in chloroform. The mixture was vigorously stirred for 5 minutes then filtered. The filter cake was washed with MeOH (10 mL), and the filtrate was concentrated in vacuo to provide ethyl hydrogen ((3-bromo-5-carbamimidoyl-7-(4,4,4-trifluorobutoxy)benzo[b]thiophen-2-yl)difluoromethyl)phosphonate, which was taken on without purification. MS (ESI, m/z): 539, 541 (M+H)⁺

### Step B: Synthesis of ((3-bromo-5-carbamimidoyl-7-(4,4,4-trifluorobutoxy)benzo[b]thiophen-2-yl)difluoromethyl)phosphonic acid

To a solution of ethyl hydrogen ((3-bromo-5-carbamimidoyl-7-(4,4,4-trifluorobutoxy)benzo[b]thiophen-2-yl)difluoromethyl)phosphonate (30 mg, 0.056 mmol) in DMF (0.3 mL) was added TMS-Br (0.22 mL, 1.7 mmol) under argon. The resulting solution was allowed to stir for 1 hour at 60° C. The reaction was cooled to room temperature, quenched by addition of EtOH (0.1 mL), and concentrated in vacuo. The resulting residue was purified using reverse phase HPLC (eluting acetonitrile in water with TFA modifier) to provide ((3-bromo-5-carbamimidoyl-7-(4,4,4-trifluorobutoxy)benzo[b]thiophen-2-yl)difluoromethyl)phosphonic acid (compound 262). ¹H NMR (400 MHz, DMSO-d₆) δ 9.53 (br s, 1H), 9.17 (br s, 1H), 7.97 (s, 1H), 7.47 (s, 1H), 4.40 (t, J=6.0 Hz, 2H), 2.48-2.40 (m, 2H), 2.15-2.04 (m, 2H). MS (ESI, m/z): 511, 513 (M+H)⁺

### Example 32

### Preparation of Compound 263

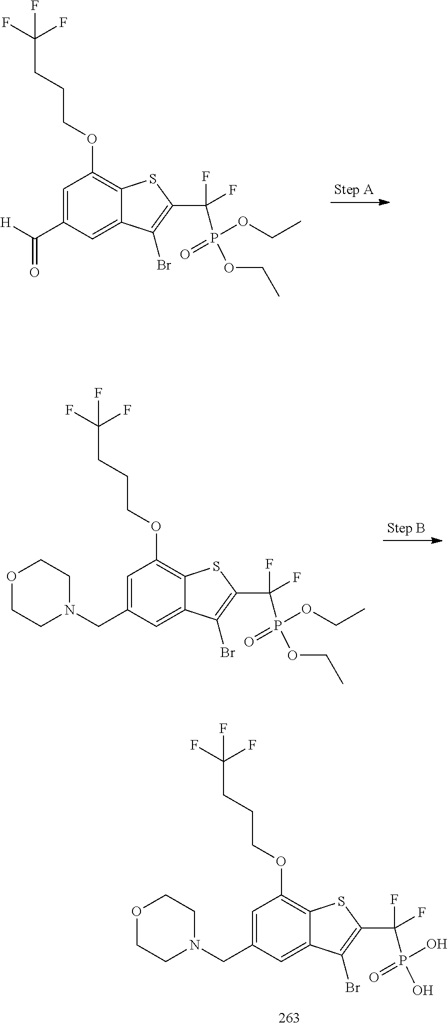 [SMILES: CCOP(=O)(OCC)C(F)(F)c1sc2c(OCCCC(F)(F)F)cc(C=O)cc2c1Br ; CCOP(=O)(OCC)C(F)(F)c1sc2c(OCCCC(F)(F)F)cc(CN3CCOCC3)cc2c1Br ; O=P(O)(O)C(F)(F)c1sc2c(OCCCC(F)(F)F)cc(CN3CCOCC3)cc2c1Br]

### Step A: Synthesis of diethyl ((3-bromo-5-(morpholinomethyl)-7-(4,4,4-trifluorobutoxy)benzo[b]thiophen-2-yl)difluoromethyl)phosphonate

Sodium triacetoxyborohydride (29 mg, 0.14 mmol) was added to a mixture of diethyl ((3-bromo-5-formyl-7-(4,4,4-trifluorobutoxy)benzo[b]thiophen-2-yl)difluoromethyl)phosphonate (30 mg, 0.054 mmol), and morpholine (0.028 mL, 0.33 mmol) in AcOH (6 μL, 0.1 mmol), and DCE (1 mL) at room temperature. The mixture was allowed to stir for 18 hours at room temperature. The mixture was filtered, and the filtrate as concentrated in vacuo to provide diethyl ((3-bromo-5-(morpholinomethyl)-7-(4,4,4-trifluorobutoxy)benzo[b]thiophen-2-yl)difluoromethyl) phosphonate, which was used without purification in the next step.

### Step B: Synthesis of ((3-bromo-5-(morpholinomethyl)-7-(4,4,4-trifluorobutoxy)benzo[b]thiophen-2-yl)difluoromethyl)phosphonic acid

TMSBr (406 μL, 3.07 mmol) was added to a mixture of diethyl ((3-bromo-5-(morpholinomethyl)-7-(4,4,4-trifluorobutoxy)benzo[b]thiophen-2-yl)difluoromethyl)phosphonate (34 mg, 0.054 mmol) in DMF (1 mL). The resulting reaction was heated to 60° C. for 90 minutes. The mixture was cooled to room temperature, and quenched with methanol (0.5 mL). The filtrate was purified using reverse phase HPLC (eluting acetonitrile in water, with TFA modifier) to provide ((3-bromo-5-(morpholinomethyl)-7-(4,4,4-trifluorobutoxy)benzo[b]thiophen-2-yl)difluoromethyl)phosphonic acid (compound 263). ¹H NMR (499 MHz, DMSO-d₆) δ 7.69 (s, 1H), 7.29 (s, 1H), 4.55 (s, 2H), 4.32 (t, J=6.1 Hz, 2H), 3.97 (d, J=12.0 Hz, 2H), 3.64 (t, J=12.2 Hz, 2H), 3.30 (d, J=12.2 Hz, 2H), 3.20-3.13 (m, 2H), 2.53-2.42 (m, 2H), 2.12-2.04 (m, 2H). MS (ESI, m/z): 568, 570 (M+H)⁺

### Example 33

### Preparation of Compound 264

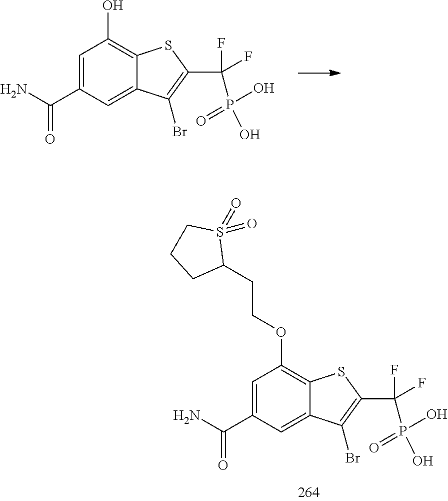 [SMILES: NC(=O)c1cc(O)c2sc(C(F)(F)P(=O)(O)O)c(Br)c2c1 ; NC(=O)c1cc(OCCC2CCCS2(=O)=O)c2sc(C(F)(F)P(=O)(O)O)c(Br)c2c1]

A mixture of diethyl ((3-bromo-5-carbamoyl-7-hydroxybenzo[b]thiophen-2-yl)difluoromethyl)phosphonate (20 mg, 0.044 mmol), 2-(2-hydroxyethyl)tetrahydrothiophene 1,1-dioxide (16 mg, 0.10 mmol), and tributylphosphoranylidene)acetonitrile (15 mg, 0.060 mmol) in toluene (0.40 mL) was stirred and heated at 80° C. for 20 minutes. The mixture was cooled to room temperature, and concentrated in vacuo, and the resulting residue was diluted with DMF (0.5 mL), and TMS-Br (0.11 mL, 0.88 mmol) was added to the mixture. The resulting reaction was heated to 60° C. for 90 minutes. The mixture was cooled to room temperature, quenched with methanol, and purified using reverse phase HPLC (eluting acetonitrile in water, with TFA modifier) to provide ((3-bromo-5-carbamoyl-7-(2-(1,1-dioxidotetrahydrothiophen-2-yl)ethoxy)benzo[b]thiophen-2-yl)difluoromethyl)phosphonic acid (compound 264). ¹H NMR (600 MHz, DMSO-d₆) δ 8.29 (s, 1H), 8.06 (s, 1H), 7.61 (s, 1H), 7.56 (s, 1H), 4.42 (t, J=6.2 Hz, 2H), 3.25-3.17 (m, 2H), 3.07-3.02 (m, 1H), 2.46-2.40 (m, 1H), 2.33-2.26 (m, 1H), 2.16-1.98 (m, 3H), 1.83-1.75 (m, 1H). MS (ESI, m/z): 548, 550 (M+H)⁺

The following illustrative compounds were made using the methods described in the Example immediately above, and substituting the appropriate reactants and/or reagents.

| | | Mass |
|---|---|---|
| Compound | Structure | [M + H]⁺ |
| 265 | 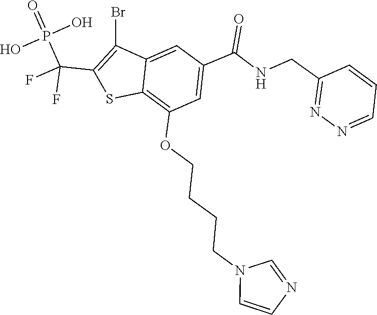 [SMILES: O=C(NCc1cccnn1)c1cc(OCCCCn2ccnc2)c2sc(C(F)(F)P(=O)(O)O)c(Br)c2c1] | 616, 618 |
| 266 | 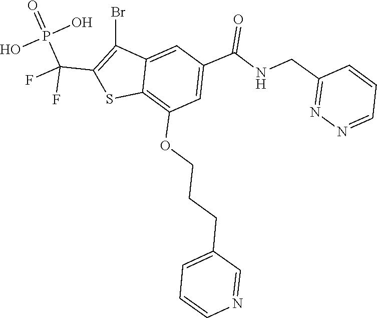 [SMILES: O=C(NCc1cccnn1)c1cc(OCCCc2cccnc2)c2sc(C(F)(F)P(=O)(O)O)c(Br)c2c1] | 613, 615 |
| 267 | 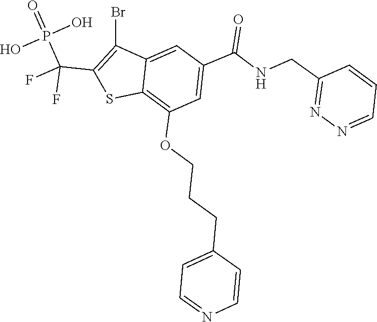 [SMILES: O=C(NCc1cccnn1)c1cc(OCCCc2ccncc2)c2sc(C(F)(F)P(=O)(O)O)c(Br)c2c1] | 613, 615 |
| 268 | 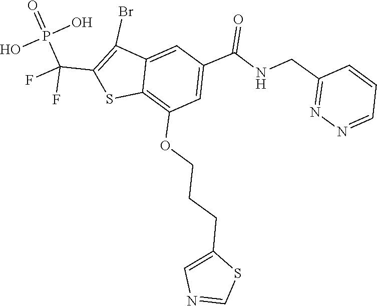 [SMILES: O=C(NCc1cccnn1)c1cc(OCCCc2cncs2)c2sc(C(F)(F)P(=O)(O)O)c(Br)c2c1] | 619, 621 |
| 269 | 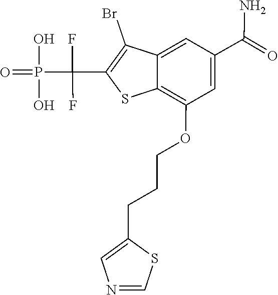 [SMILES: NC(=O)c1cc(OCCCc2cncs2)c2sc(C(F)(F)P(=O)(O)O)c(Br)c2c1] | 527, 529 |
| 270 | 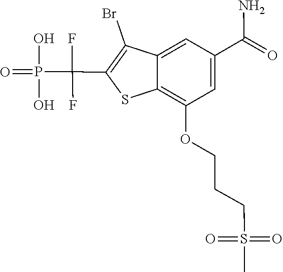 [SMILES: CS(=O)(=O)CCCOc1cc(C(N)=O)cc2c(Br)c(C(F)(F)P(=O)(O)O)sc12] | 522. 524 |
| 271 | 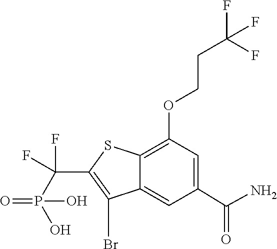 [SMILES: NC(=O)c1cc(OCCC(F)(F)F)c2sc(C(F)(F)P(=O)(O)O)c(Br)c2c1] | 498, 500 |
| 272 | 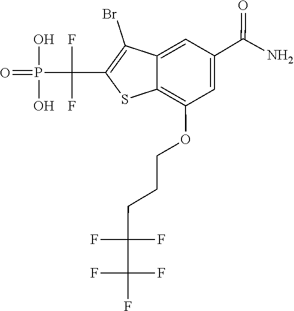 [SMILES: NC(=O)c1cc(OCCCC(F)(F)C(F)(F)F)c2sc(C(F)(F)P(=O)(O)O)c(Br)c2c1] | 562, 564 |
| 273 | 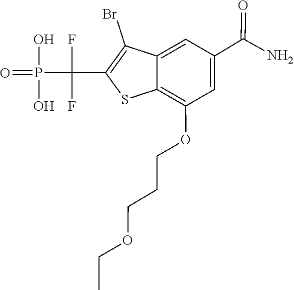 [SMILES: CCOCCCOc1cc(C(N)=O)cc2c(Br)c(C(F)(F)P(=O)(O)O)sc12] | 488, 490 |
| 274 | 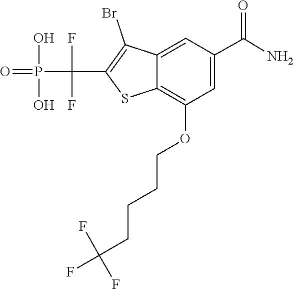 [SMILES: NC(=O)c1cc(OCCCCC(F)(F)F)c2sc(C(F)(F)P(=O)(O)O)c(Br)c2c1] | 526, 528 |
| 275 | 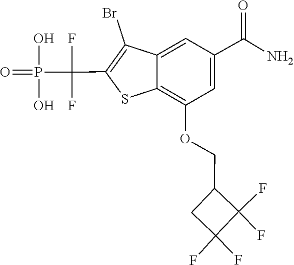 [SMILES: NC(=O)c1cc(OCC2CC(F)(F)C2(F)F)c2sc(C(F)(F)P(=O)(O)O)c(Br)c2c1] | 542, 544 |
| 276 | 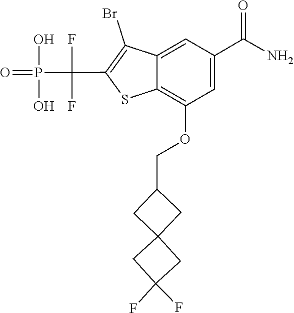 [SMILES: NC(=O)c1cc(OCC2CC3(C2)CC(F)(F)C3)c2sc(C(F)(F)P(=O)(O)O)c(Br)c2c1] | 546, 548 |
| 277 | 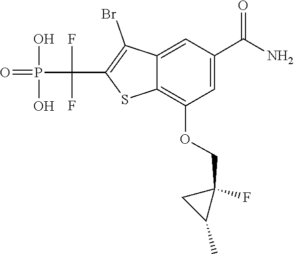 [SMILES: C[C@@H]1C[C@@]1(F)COc1cc(C(N)=O)cc2c(Br)c(C(F)(F)P(=O)(O)O)sc12] | 488, 490 |
| 278 | 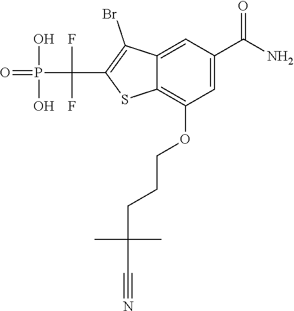 [SMILES: CC(C)(C#N)CCCOc1cc(C(N)=O)cc2c(Br)c(C(F)(F)P(=O)(O)O)sc12] | 511, 513 |
| 279 | 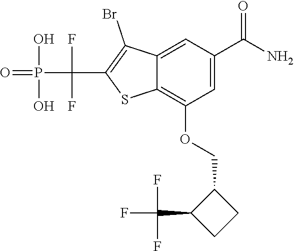 [SMILES: NC(=O)c1cc(OC[C@@H]2CC[C@H]2C(F)(F)F)c2sc(C(F)(F)P(=O)(O)O)c(Br)c2c1] | 538, 540 |
| 280 | 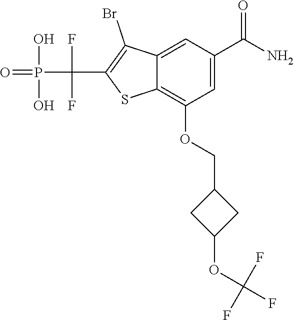 [SMILES: NC(=O)c1cc(OCC2CC(OC(F)(F)F)C2)c2sc(C(F)(F)P(=O)(O)O)c(Br)c2c1] | 554, 556 |
| 281 | 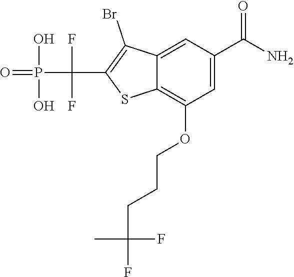 [SMILES: CC(F)(F)CCCOc1cc(C(N)=O)cc2c(Br)c(C(F)(F)P(=O)(O)O)sc12] | 508, 510 |
| 282 | 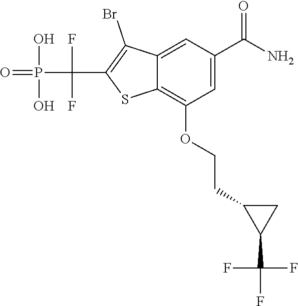 [SMILES: NC(=O)c1cc(OCC[C@@H]2C[C@H]2C(F)(F)F)c2sc(C(F)(F)P(=O)(O)O)c(Br)c2c1] | 538, 540 |
| 283 | 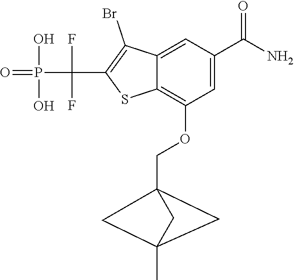 [SMILES: CC12CC(COc3cc(C(N)=O)cc4c(Br)c(C(F)(F)P(=O)(O)O)sc34)(C1)C2] | 496, 498 |
| 284 | 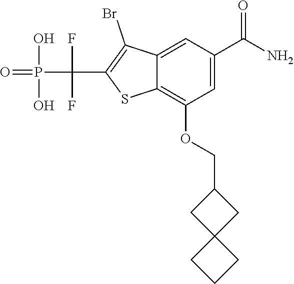 [SMILES: NC(=O)c1cc(OCC2CC3(CCC3)C2)c2sc(C(F)(F)P(=O)(O)O)c(Br)c2c1] | 510, 512 |
| 285 | 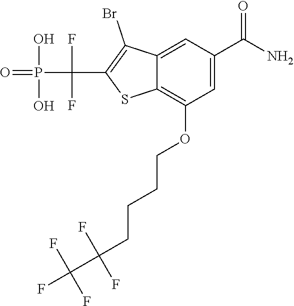 [SMILES: NC(=O)c1cc(OCCCCC(F)(F)C(F)(F)F)c2sc(C(F)(F)P(=O)(O)O)c(Br)c2c1] | 576, 578 |
| 286 | 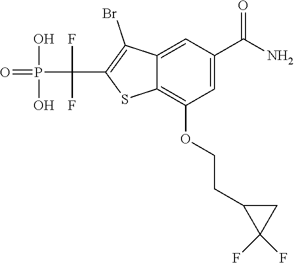 [SMILES: NC(=O)c1cc(OCCC2CC2(F)F)c2sc(C(F)(F)P(=O)(O)O)c(Br)c2c1] | 506, 508 |
| 287 | 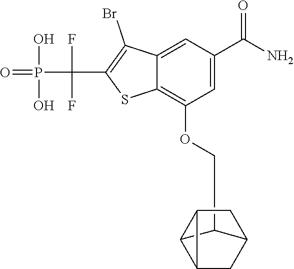 [SMILES: NC(=O)c1cc(OCC2C3CC4C(C3)C24)c2sc(C(F)(F)P(=O)(O)O)c(Br)c2c1] | 508, 510 |
| 288 | 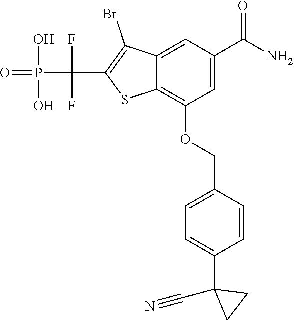 [SMILES: N#CC1(c2ccc(COc3cc(C(N)=O)cc4c(Br)c(C(F)(F)P(=O)(O)O)sc34)cc2)CC1] | 557, 559 |
| 289 | 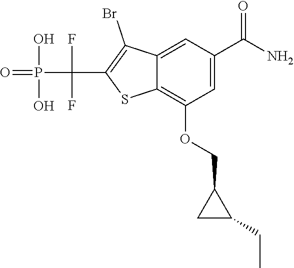 [SMILES: CC[C@@H]1C[C@H]1COc1cc(C(N)=O)cc2c(Br)c(C(F)(F)P(=O)(O)O)sc12] | 484, 486 |
| 290 | 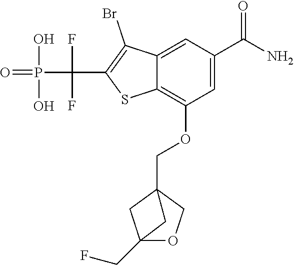 [SMILES: NC(=O)c1cc(OCC23COC(CF)(C2)C3)c2sc(C(F)(F)P(=O)(O)O)c(Br)c2c1] | 530, 532 |
| 291 | 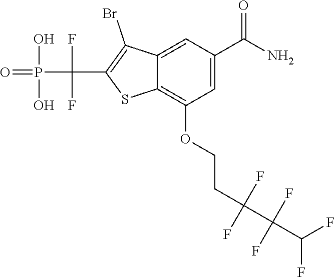 [SMILES: NC(=O)c1cc(OCCC(F)(F)C(F)(F)C(F)F)c2sc(C(F)(F)P(=O)(O)O)c(Br)c2c1] | 580, 582 |
| 292 | 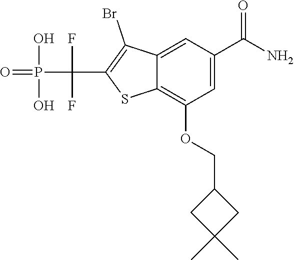 [SMILES: CC1(C)CC(COc2cc(C(N)=O)cc3c(Br)c(C(F)(F)P(=O)(O)O)sc23)C1] | 498, 500 |
| 293 | 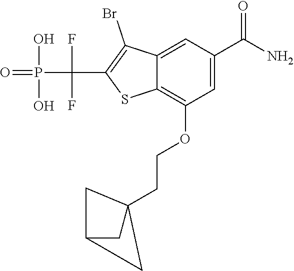 [SMILES: NC(=O)c1cc(OCCC23CC(C2)C3)c2sc(C(F)(F)P(=O)(O)O)c(Br)c2c1] | 496, 498 |
| 294 | 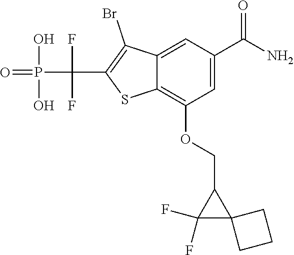 [SMILES: NC(=O)c1cc(OCC2C(F)(F)C23CCC3)c2sc(C(F)(F)P(=O)(O)O)c(Br)c2c1] | 532, 534 |
| 295 | 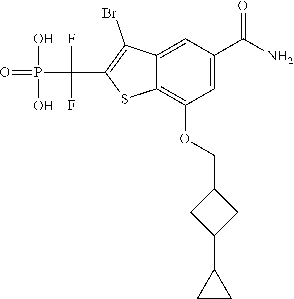 [SMILES: NC(=O)c1cc(OCC2CC(C3CC3)C2)c2sc(C(F)(F)P(=O)(O)O)c(Br)c2c1] | 510, 512 |
| 370 | 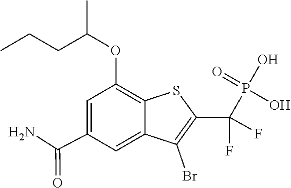 [SMILES: CCCC(C)Oc1cc(C(N)=O)cc2c(Br)c(C(F)(F)P(=O)(O)O)sc12] | 472, 474 |
| 371^{a} | 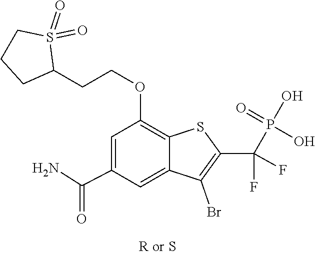 [SMILES: NC(=O)c1cc(OCCC2CCCS2(=O)=O)c2sc(C(F)(F)P(=O)(O)O)c(Br)c2c1] | 548, 550 |
| 372^{a} | 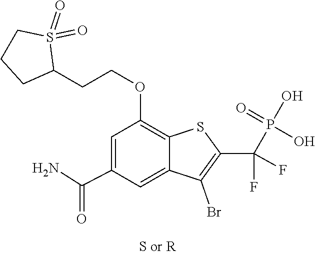 [SMILES: NC(=O)c1cc(OCCC2CCCS2(=O)=O)c2sc(C(F)(F)P(=O)(O)O)c(Br)c2c1] | 548, 550 |
| 373 | 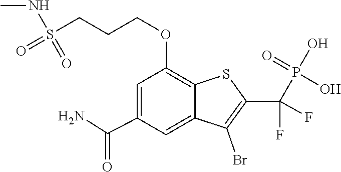 [SMILES: CNS(=O)(=O)CCCOc1cc(C(N)=O)cc2c(Br)c(C(F)(F)P(=O)(O)O)sc12] | 537, 539 |
| 374 | 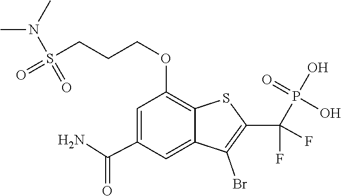 [SMILES: CN(C)S(=O)(=O)CCCOc1cc(C(N)=O)cc2c(Br)c(C(F)(F)P(=O)(O)O)sc12] | 551, 553 |
| 375 | 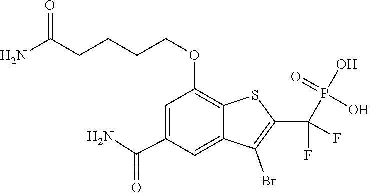 [SMILES: NC(=O)CCCCOc1cc(C(N)=O)cc2c(Br)c(C(F)(F)P(=O)(O)O)sc12] | 501, 503 |
| 376 | 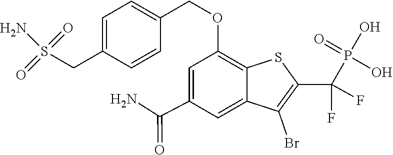 [SMILES: NC(=O)c1cc(OCc2ccc(CS(N)(=O)=O)cc2)c2sc(C(F)(F)P(=O)(O)O)c(Br)c2c1] | 585, 587 |
| 377 | 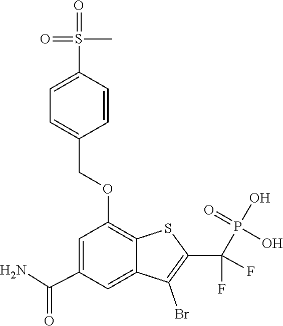 [SMILES: CS(=O)(=O)c1ccc(COc2cc(C(N)=O)cc3c(Br)c(C(F)(F)P(=O)(O)O)sc23)cc1] | 570, 572 |
| 378 | 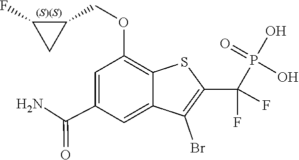 | 474, 476 |
| 379 | 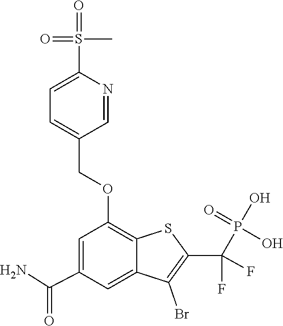 [SMILES: CS(=O)(=O)c1ccc(COc2cc(C(N)=O)cc3c(Br)c(C(F)(F)P(=O)(O)O)sc23)cn1] | 571, 573 |
| 380 | 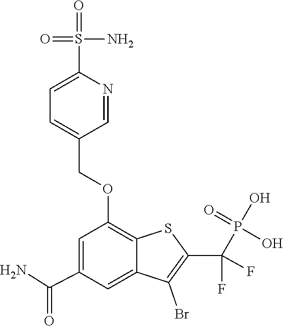 [SMILES: NC(=O)c1cc(OCc2ccc(S(N)(=O)=O)nc2)c2sc(C(F)(F)P(=O)(O)O)c(Br)c2c1] | 570, 572 [M − H]⁻ |
| 381 | 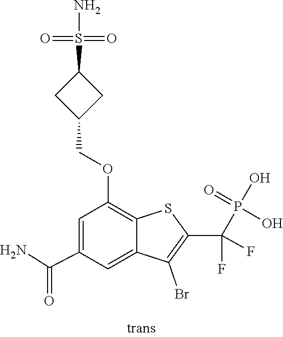 [SMILES: NC(=O)c1cc(OC[C@H]2C[C@H](S(N)(=O)=O)C2)c2sc(C(F)(F)P(=O)(O)O)c(Br)c2c1] | 549, 551 |
| 382 | 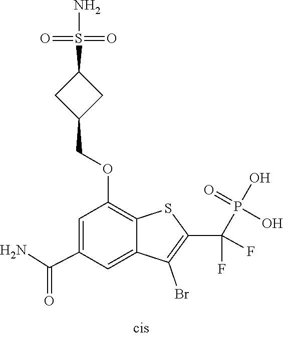 [SMILES: NC(=O)c1cc(OC[C@H]2C[C@@H](S(N)(=O)=O)C2)c2sc(C(F)(F)P(=O)(O)O)c(Br)c2c1] | 549, 551 |
| 383 | 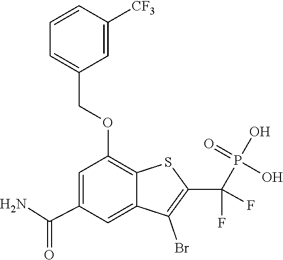 [SMILES: NC(=O)c1cc(OCc2cccc(C(F)(F)F)c2)c2sc(C(F)(F)P(=O)(O)O)c(Br)c2c1] | 560, 562 |
| 384 | 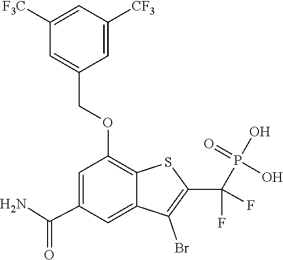 [SMILES: NC(=O)c1cc(OCc2cc(C(F)(F)F)cc(C(F)(F)F)c2)c2sc(C(F)(F)P(=O)(O)O)c(Br)c2c1] | 628, 630 |
| 385 | 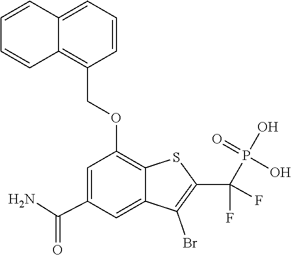 [SMILES: NC(=O)c1cc(OCc2cccc3ccccc23)c2sc(C(F)(F)P(=O)(O)O)c(Br)c2c1] | 542, 544 |
| 386 | 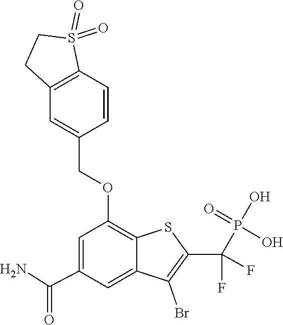 [SMILES: NC(=O)c1cc(OCc2ccc3c(c2)CCS3(=O)=O)c2sc(C(F)(F)P(=O)(O)O)c(Br)c2c1] | 582, 584 |
| 387 | 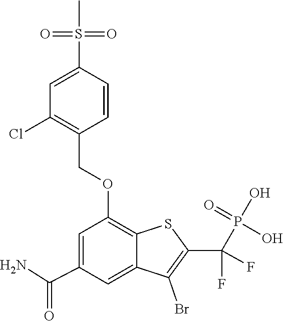 [SMILES: CS(=O)(=O)c1ccc(COc2cc(C(N)=O)cc3c(Br)c(C(F)(F)P(=O)(O)O)sc23)c(Cl)c1] | 602, 604 [M − H]⁻ |
| 388 | 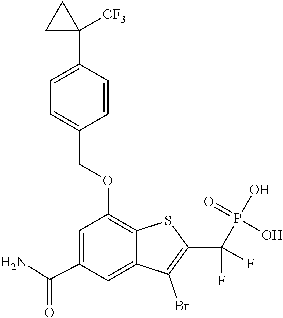 [SMILES: NC(=O)c1cc(OCc2ccc(C3(C(F)(F)F)CC3)cc2)c2sc(C(F)(F)P(=O)(O)O)c(Br)c2c1] | 600, 602 |
| 389 | 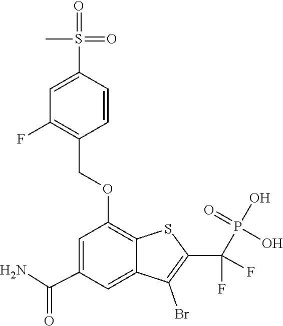 [SMILES: CS(=O)(=O)c1ccc(COc2cc(C(N)=O)cc3c(Br)c(C(F)(F)P(=O)(O)O)sc23)c(F)c1] | 588, 590 |
| 390 | 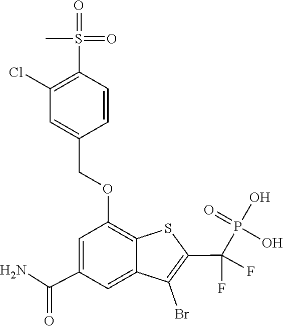 [SMILES: CS(=O)(=O)c1ccc(COc2cc(C(N)=O)cc3c(Br)c(C(F)(F)P(=O)(O)O)sc23)cc1Cl] | 604, 606 |
| 391 | 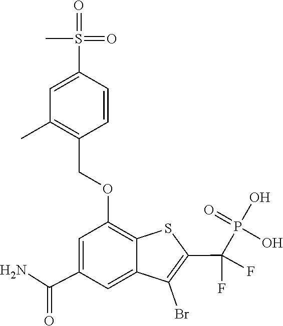 [SMILES: Cc1cc(S(C)(=O)=O)ccc1COc1cc(C(N)=O)cc2c(Br)c(C(F)(F)P(=O)(O)O)sc12] | 582, 584 [M − H]⁻ |
| 392 | 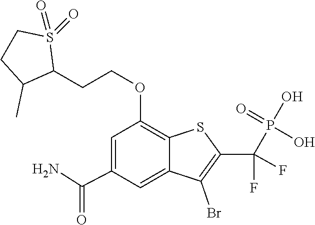 [SMILES: CC1CCS(=O)(=O)C1CCOc1cc(C(N)=O)cc2c(Br)c(C(F)(F)P(=O)(O)O)sc12] | 562, 564 |
| 393^{b1} | 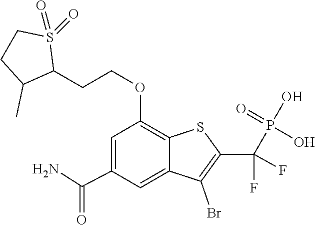 [SMILES: CC1CCS(=O)(=O)C1CCOc1cc(C(N)=O)cc2c(Br)c(C(F)(F)P(=O)(O)O)sc12] | 562, 564 |
| 394^{b2} | 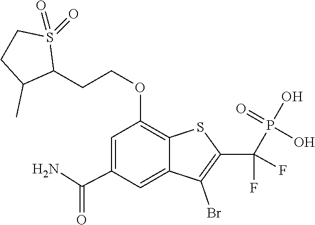 [SMILES: CC1CCS(=O)(=O)C1CCOc1cc(C(N)=O)cc2c(Br)c(C(F)(F)P(=O)(O)O)sc12] | 562, 564 |
| 395^{b2} | 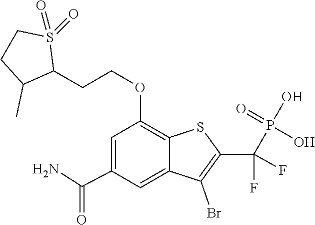 [SMILES: CC1CCS(=O)(=O)C1CCOc1cc(C(N)=O)cc2c(Br)c(C(F)(F)P(=O)(O)O)sc12] | 562, 564 |
| 396 | 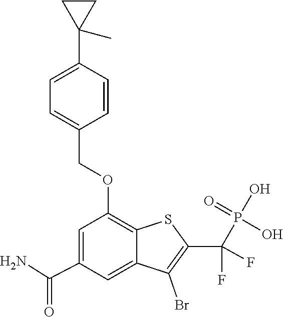 [SMILES: CC1(c2ccc(COc3cc(C(N)=O)cc4c(Br)c(C(F)(F)P(=O)(O)O)sc34)cc2)CC1] | 544, 546 [M − H]⁻ |
| 397 | 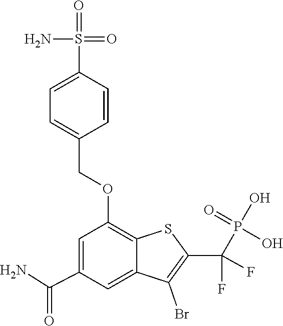 [SMILES: NC(=O)c1cc(OCc2ccc(S(N)(=O)=O)cc2)c2sc(C(F)(F)P(=O)(O)O)c(Br)c2c1] | 571, 573 |
| 398 | 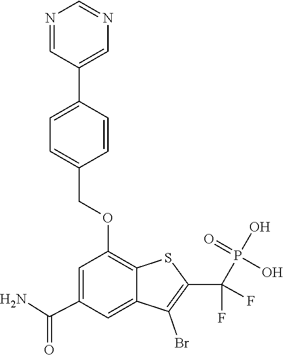 [SMILES: NC(=O)c1cc(OCc2ccc(-c3cncnc3)cc2)c2sc(C(F)(F)P(=O)(O)O)c(Br)c2c1] | 568, 570 [M − H]⁻ |
| 399 | 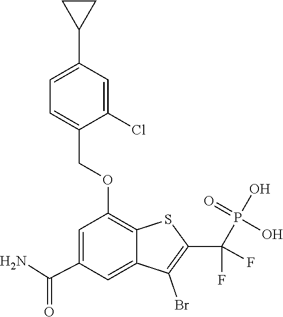 [SMILES: NC(=O)c1cc(OCc2ccc(C3CC3)cc2Cl)c2sc(C(F)(F)P(=O)(O)O)c(Br)c2c1] | 564, 566 [M − H]⁻ |
| 400 | 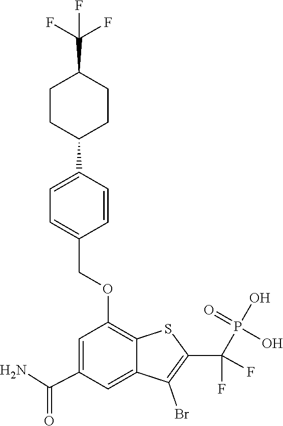 [SMILES: NC(=O)c1cc(OCc2ccc([C@H]3CC[C@H](C(F)(F)F)CC3)cc2)c2sc(C(F)(F)P(=O)(O)O)c(Br)c2c1] | 640, 642 [M − H]⁻ |
| 401 | 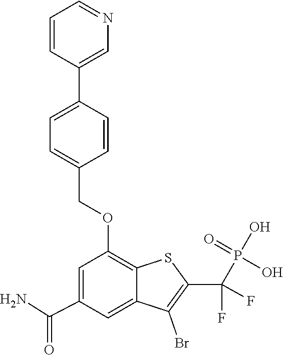 [SMILES: NC(=O)c1cc(OCc2ccc(-c3cccnc3)cc2)c2sc(C(F)(F)P(=O)(O)O)c(Br)c2c1] | 567, 569 [M − H]⁻ |
| 402 | 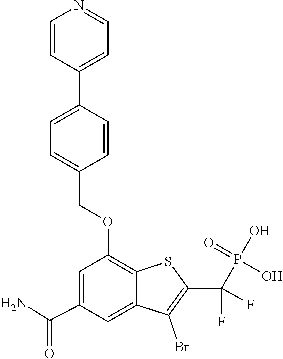 [SMILES: NC(=O)c1cc(OCc2ccc(-c3ccncc3)cc2)c2sc(C(F)(F)P(=O)(O)O)c(Br)c2c1] | 567, 569 [M − H]⁻ |
| 403 | 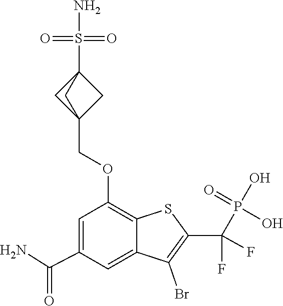 [SMILES: NC(=O)c1cc(OCC23CC(S(N)(=O)=O)(C2)C3)c2sc(C(F)(F)P(=O)(O)O)c(Br)c2c1] | 559, 561 [M − H]⁻ |

^{a}Products were separated into pure stereoisomers prior to TMS-Br deprotection using the following condtions: ChiralPak AD-H; 21 xIn 250 mm, 5 um; 30% methanol in CO₂ with 0.1% ammonium hydroxide; flow rate = 70 mL/min
^{b1}Products were separated into pure stereoisomers prior to TMS-Br deprotection using the following condtions: ChiralPak IE; 21 x 250 mm, 5 um; 20% [(0.5% ammonia/MeOH) in MTBE] in isopropanol; flow rate = 20 mL/min, followed by Chiral Art Cellulose-SB; 21 x 250 mm, 5 um; 80% [(0.5% ammonia/MeOH) in MTBE] in ethanol; flow rate = 20 mL/min
^{b2}Products were separated into pure stereoisomers prior to TMS-Br deprotection using the following condtions: ChiralPak IE; 21 x 250 mm, 5 um; 20% [(0.5% ammonia/MeOH) in MTBE] in isopropanol; flow rate = 20 mL/min, followed by Lux Cellulose-3; 21 x 250 mm, 5 um; 30% [(0.5% ammonia/MeOH) in hexane] in ethanol; flow rate = 20 mL/min

### Example 34

### Preparation of Compound 296

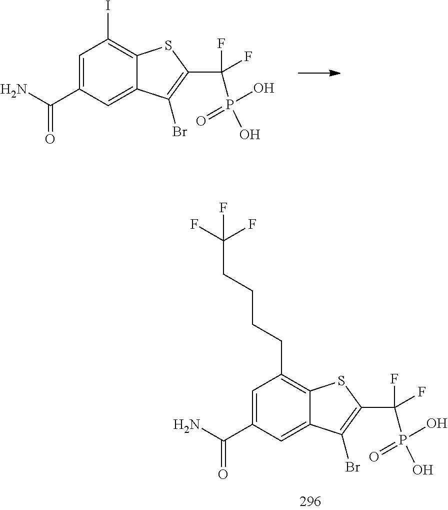 [SMILES: NC(=O)c1cc(I)c2sc(C(F)(F)P(=O)(O)O)c(Br)c2c1 ; NC(=O)c1cc(CCCCC(F)(F)F)c2sc(C(F)(F)P(=O)(O)O)c(Br)c2c1]

A mixture of (5,5,5-trifluoropentyl)zinc (II) bromide (0.25 M in THF, 0.900 mL, 0.224 mmol) was added to a mixture of Int-13 (20 mg, 0.035 mmol), and CPhos Pd G4 (6 mg, 7 μmol) in THF (352 mL) at room temperature. The resulting reaction was heated to 40° C. for 3 hours. The mixture was concentrated in vacuo and then diluted with DMF (0.4 mL). TMS-Br (91 μl, 0.704 mmol) was added and the mixture was stirred and heated to 60° C. for 1 hour. The mixture was cooled to room temperature, and quenched with MeOH. The mixture was purified using reverse phase HPLC (eluting acetonitrile in water, with 0.1% TFA modifier) to provide ((3-bromo-5-carbamoyl-7-(5,5,5-trifluoropentyl)benzo[b]thiophen-2-yl)difluoromethyl)phosphonic acid (compound 296). ¹H NMR (600 MHz, DMSO-d₆) δ 8.26 (s, 2H), 7.90 (s, 1H), 7.49 (s, 1H), 2.95-2.89 (m, 2H), 2.36-2.27 (m, 2H), 1.86-1.79 (m, 2H), 1.62-1.53 (m, 2H). MS (ESI, m/z): 510, 512 (M+H)⁺

### Example 35

### Preparation of Compound 297

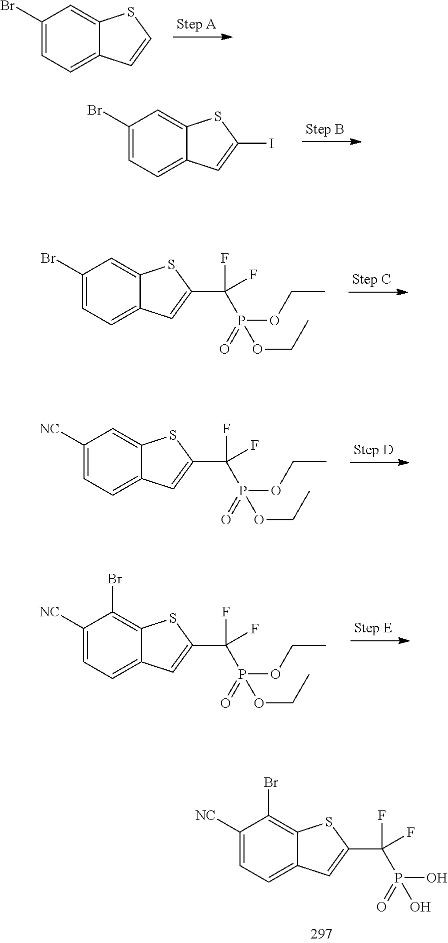 [SMILES: Brc1ccc2ccsc2c1 ; Brc1ccc2cc(I)sc2c1 ; CCOP(=O)(OCC)C(F)(F)c1cc2ccc(Br)cc2s1 ; CCOP(=O)(OCC)C(F)(F)c1cc2ccc(C#N)cc2s1 ; CCOP(=O)(OCC)C(F)(F)c1cc2ccc(C#N)c(Br)c2s1 ; N#Cc1ccc2cc(C(F)(F)P(=O)(O)O)sc2c1Br]

### Step A: Synthesis of 6-bromo-2-iodobenzo[b]thiophene

LDA (1.0 M in THF, 5.4 mL, 5.4 mmol) was added to a solution of 6-bromobenzo[b]thiophene (1.04 g, 4.88 mmol) in THF (20 ml) at −78° C. The mixture was allowed to stir at −78° C. for 3 hours. Iodine (1.86 g, 7.31 mmol) was added to the mixture at −78° C., and the mixture was warmed to room temperature, and stirred for 18 hours. The mixture was slowly quenched by the addition of saturated aqueous sodium thiosulfate (10 mL). The mixture was diluted with EtOAc (10 mL). The organic layer was separated, washed with brine (3×), dried over sodium sulfate, filtered, and concentrated in vacuo, and the resulting residue was purified using silica gel chromatography (eluting with neat hexanes) to provide 6-bromo-2-iodobenzo[b]thiophene. ¹H NMR (499 MHz, chloroform-d) δ 7.93 (s, 1H), 7.58 (d, J=8.5 Hz, 1H), 7.51 (s, 1H), 7.46-7.41 (m, 1H).

### Step B: Synthesis of diethyl ((6-bromobenzo[b]thiophen-2-yl)difluoromethyl)phosphonate

A mixture of copper (I) bromide (192 mg, 1.34 mmol), and 6-bromo-2-iodobenzo[b]thiophene (227 mg, 0.670 mmol) was purged with argon (3×). A solution of ((diethoxyphosphoryl)difluoromethyl)cadmium (1.0 M in DMF, 2.0 mL, 2.0 mmol) was added to the mixture, and the mixture was allowed to stir at room temperature for 18 hours. The reaction was quenched with 1M HCl and filtered through Celite. The filtrate was washed with water (3×), and the organic layer was separated, dried over sodium sulfate, filtered, and concentrated in vacuo, and the resulting residue was purified using silica gel chromatography (eluting (1:3) EtOH/EtOAc in hexanes to provide diethyl ((6-bromobenzo[b]thiophen-2-yl)difluoromethyl)phosphonate. MS (ESI, m/z): 399, 401 (M+H)⁺

### Step C: Synthesis of diethyl ((6-cyanobenzo[b]thiophen-2-yl)difluoromethyl)phosphonate

A mixture of diethyl ((6-bromobenzo[b]thiophen-2-yl)difluoromethyl)phosphonate (112 mg, 0.281 mmol), dicyanozinc (22 mg, 0.19 mmol), and tert-butyl X-phos Pd G3 (19 mg, 0.028 mmol) was purged with argon (3×). THF (1.0 mL), and water (0.2 mL) were added and the reaction was stirred and heated at 40° C. for 18 hours. The mixture was cooled to room temperature, and concentrated in vacuo, and the resulting residue was purified using silica gel chromatography (eluting (1:3) EtOH/EtOAc in hexanes) to provide diethyl ((6-cyanobenzo[b]thiophen-2-yl)difluoromethyl)phosphonate.

### Step D: Synthesis of diethyl ((7-bromo-6-cyanobenzo[b]thiophen-2-yl)difluoromethyl)phosphonate

NBS (14 mg, 0.076 mmol) was added to a solution of diethyl ((6-cyanobenzo[b]thiophen-2-yl)difluoromethyl)phosphonate (24 mg, 0.069 mmol) in TFA (2.0 mL). The resulting reaction was heated to 50° C. for 3 days. The mixture was cooled to room temperature, and concentrated in vacuo, and the resulting residue was taken up in DCM (2 mL), and washed with saturated aqueous sodium bicarbonate. The organic layer was separated, dried over magnesium sulfate, filtered, and concentrated in vacuo, and the resulting residue was purified using silica gel chromatography (eluting (1:3) EtOH/EtOAc in hexanes) to provide diethyl ((7-bromo-6-cyanobenzo[b]thiophen-2-yl)difluoromethyl)phosphonate. MS (ESI, m/z): 424, 426 (M+H)⁺

### Step E: Synthesis of ((7-bromo-6-cyanobenzo[b]thiophen-2-yl)difluoromethyl)phosphonic acid

TMS-Br (500 μl, 3.85 mmol) was added to a mixture of diethyl ((7-bromo-6-cyanobenzo[b]thiophen-2-yl)difluoromethyl)phosphonate in DCM (1 mL). The mixture was stirred and heated to 50° C. for 4 hours. The mixture was concentrated in vacuo, diluted with MeOH (0.1 mL), and again concentrated in vacuo, and the resulting residue was purified using reverse phase HPLC (eluting acetonitrile in water, with 0.1% TFA modifier) to provide ((7-bromo-6-cyanobenzo[b]thiophen-2-yl)difluoromethyl)phosphonic acid (compound 297). ¹H NMR (499 MHz, chloroform-d) δ 8.21 (s, 1H), 8.06 (d, J=8.5 Hz, 1H), 7.76 (dd, J=8.5, 1.3 Hz, 1H).

### Example 36

### Preparation of Compound 298

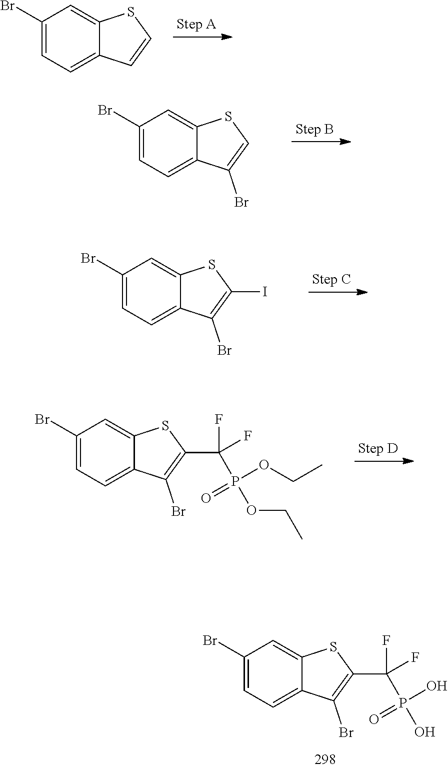 [SMILES: Brc1ccc2ccsc2c1 ; Brc1ccc2c(Br)csc2c1 ; Brc1ccc2c(Br)c(I)sc2c1 ; CCOP(=O)(OCC)C(F)(F)c1sc2cc(Br)ccc2c1Br ; O=P(O)(O)C(F)(F)c1sc2cc(Br)ccc2c1Br]

### Step A: Synthesis of 3,6-dibromobenzo[b]thiophene

To a mixture of 6-bromobenzo[b]thiophene (7700 mg, 36 mmol) in DCM (20 mL), and acetic acid (20 mL) was added N-bromosuccinimide (7100 mg, 40 mmol). The reaction was allowed to stir for 18 hours at 50° C. then concentrated in vacuo, and the resulting residue was purified using silica gel chromatography (eluting with petroleum ether) to provide 3,6-dibromobenzo[b]thiophene. ¹H NMR (499 MHz, CDCl₃) δ 8.01 (d, J=1.4 Hz, 1H), 7.70 (d, J=8.6 Hz, 1H), 7.58 (dd, J=8.6, 1.6 Hz, 1H), 7.43 (s, 1H).

### Step B: Synthesis of 3,6-dibromo-2-iodobenzo[b]thiophene

To a mixture of 3,6-dibromobenzo[b]thiophene (2000 mg, 7.0 mmol) in THF (20 mL) was added LDA (1.0 M in THF, 7.7 mL, 7.7 mmol) at −78° C. The mixture was allowed to stir for 3 hours at −78° C. then a solution of iodine (2700 mg, 10 mmol) in THF (10 mL) was added. The mixture was warmed to room temperature, and stirred for an additional 18 hours. The reaction was quenched by addition of saturated aqueous sodium thiosulfate (50 mL), and extracted with EtOAc (50 mL). The organic extract was washed with brine (50 mL), dried over anhydrous Na₂SO₄ and concentrated in vacuo. The resulting residue was directly purified using silica gel chromatography (eluting with petroleum ether) to provide 3,6-dibromo-2-iodobenzo[b]thiophene. ¹H NMR (499 MHz, CDCl₃) δ 7.92 (d, J=1.4 Hz, 1H), 7.64 (d, J=8.6 Hz, 1H), 7.53 (dd, J=8.6, 1.6 Hz, 1H).

### Step C: Synthesis of diethyl ((3,6-dibromobenzo[b]thiophen-2-yl)difluoromethyl)phosphonate

To a mixture of 3,6-dibromo-2-iodobenzo[b]thiophene (75 mg, 0.18 mmol), and copper(I) bromide (52 mg, 0.36 mmol) in DMF (1 mL) was added ((diethoxyphosphoryl)difluoromethyl)zinc(II) bromide (1.0 M in THF, 0.36 mL, 0.36 mmol) under argon. The mixture was allowed to stir at room temperature for 18 hours. The reaction was quenched by addition of 1M HCl (1 mL), and extracted with EtOAc (5 mL). The organic extract was washed with water (3×5 mL), dried over anhydrous Na₂SO₄ and concentrated in vacuo to provide diethyl ((3,6-dibromobenzo[b]thiophen-2-yl)difluoromethyl)phosphonate, which was used without purification in the next step.

### Step D: Synthesis of ((3,6-dibromobenzo[b]thiophen-2-yl)difluoromethyl)phosphonic acid

To a mixture of diethyl ((3,6-dibromobenzo[b]thiophen-2-yl)difluoromethyl)phosphonate (86 mg, 0.18 mmol) in DCM (2 mL) was added TMS-Br (1.0 mL, 7.7 mmol). The reaction was stirred and heated at 50° C. for 18 hours. The mixture was then cooled to room temperature, and concentrated in vacuo, and the resulting residue was taken up in MeOH (1 mL), and again concentrated in vacuo, and the resulting residue was purified using reverse phase HPLC (eluting acetonitrile in water, with 0.1% TFA modifier) to provide ((3,6-dibromobenzo[b]thiophen-2-yl)difluoromethyl)phosphonic acid (compound 298). ¹H NMR (499 MHz, DMSO-d₆) δ 8.48 (d, J=1.6 Hz, 1H), 7.81 (d, J=8.7 Hz, 1H), 7.75 (dd, J=8.7, 1.7 Hz, 1H). MS (ESI, m/z): 423 (M+H)⁺.

### Example 37

### Preparation of Compound 299

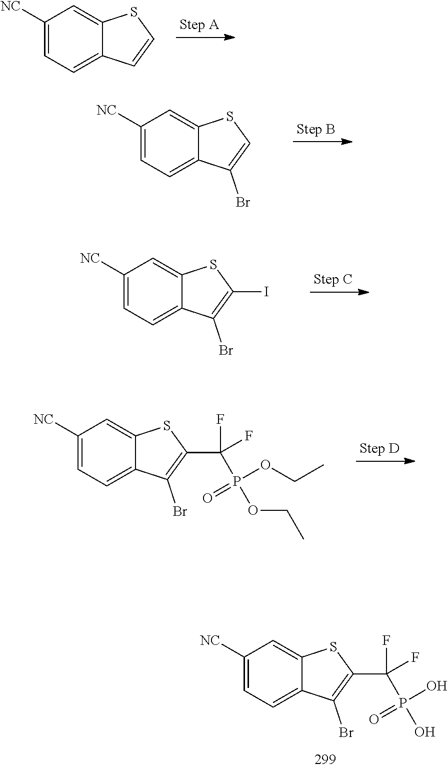 [SMILES: N#Cc1ccc2ccsc2c1 ; N#Cc1ccc2c(Br)csc2c1 ; N#Cc1ccc2c(Br)c(I)sc2c1 ; CCOP(=O)(OCC)C(F)(F)c1sc2cc(C#N)ccc2c1Br ; N#Cc1ccc2c(Br)c(C(F)(F)P(=O)(O)O)sc2c1]

### Step A: Synthesis of 3-bromobenzo[b]thiophene-6-carbonitrile

To a mixture of benzo[b]thiophene-6-carbonitrile (1.0 g, 6.3 mmol) in TFA (6 mL) was added N-bromosuccinimide (1.2 g, 7.0 mmol). The reaction was allowed to stir for 18 hours at room temperature, and then concentrated in vacuo, and the resulting residue was treated with water (20 mL) to provide a slurry. The slurry was filtered, and the solid was collected and dried in vacuo to provide 3-bromobenzo[b]thiophene-6-carbonitrile. ¹H NMR (499 MHz, CDCl₃) δ 8.22 (s, 1H), 7.96 (d, J=8.4 Hz, 1H), 7.74-7.71 (m, 2H).

### Step B: Synthesis of 3-bromo-2-iodobenzo[b]thiophene-6-carbonitrile

To a mixture of 3-bromobenzo[b]thiophene-6-carbonitrile (0.10 g, 0.42 mmol) in THF (2.1 mL) was added a solution of LDA (1.0 M in THF, 0.46 mL, 0.46 mmol) at −78° C. The mixture was allowed to stir for 3 hours at −78° C., then a solution of iodine (160 mg, 0.63 mmol) in THF (2 mL) was added. The mixture was warmed to room temperature and stirred for an additional 18 hours. The reaction was quenched by addition of saturated aqueous sodium thiosulfate (10 mL) and extracted with EtOAc (10 mL). The organic extract was washed with brine (10 mL), dried over anhydrous Na₂SO₄, filtered, and concentrated in vacuo, and the resulting residue was purified using silica gel chromatography (eluting with petroleum ether) to provide 3-bromo-2-iodobenzo[b]thiophene-6-carbonitrile. ¹H NMR (499 MHz, CDCl₃) δ 8.11 (s, 1H), 7.88 (d, J=8.4 Hz, 1H), 7.66 (d, J=8.4 Hz, 1H).

### Step C: Synthesis of diethyl ((3-bromo-6-cyanobenzo[b]thiophen-2-yl)difluoromethyl)phosphonate

To a mixture of 3-bromo-2-iodobenzo[b]thiophene-6-carbonitrile (130 mg, 0.35 mmol), and copper (I) bromide (100 mg, 0.7 mmol) in DMF (4 mL) was added a solution of ((diethoxyphosphoryl)difluoromethyl)zinc(II) bromide (1 M in THF, 0.7 mL, 0.7 mmol) under an argon atmosphere. The mixture was allowed to stir at room temperature for 18 hours. The reaction was quenched by the addition of 1M HCl (2 mL) and extracted with EtOAc (5 mL). The organic extract was washed with water (3×5 mL), dried over anhydrous Na₂SO₄, filtered, and concentrated in vacuo to provide diethyl ((3-bromo-6-cyanobenzo[b]thiophen-2-yl)difluoromethyl)phosphonate, which was used without purification in the next step.

### Step D: Synthesis of ((3-bromo-6-cyanobenzo[b]thiophen-2-yl)difluoromethyl)phosphonic acid

To a mixture of diethyl ((3-bromo-6-cyanobenzo[b]thiophen-2-yl)difluoromethyl)phosphonate (150 mg, 0.35 mmol) in DCM (2 mL) was added TMS-Br (1.0 mL, 7.7 mmol). The mixture was allowed to stir for 18 hours at 50° C., then concentrated in vacuo, and the resulting residue was diluted with MeOH (1 mL), and again concentrated in vacuo, and the resulting residue was purified using reverse phase HPLC (eluting acetonitrile in water, with 0.1% TFA modifier) to provide ((3-bromo-6-cyanobenzo[b]thiophen-2-yl)difluoromethyl)phosphonic acid (compound 299)¹H NMR (499 MHz, DMSO-d₆) δ 8.79 (s, 1H), 8.03 (d, J=8.4 Hz, 1H), 7.96 (d, J=8.4 Hz, 1H). MS (ESI, m/z): 366, 368 (M−H)⁻.

### Example 38

### Preparation of Compound 300

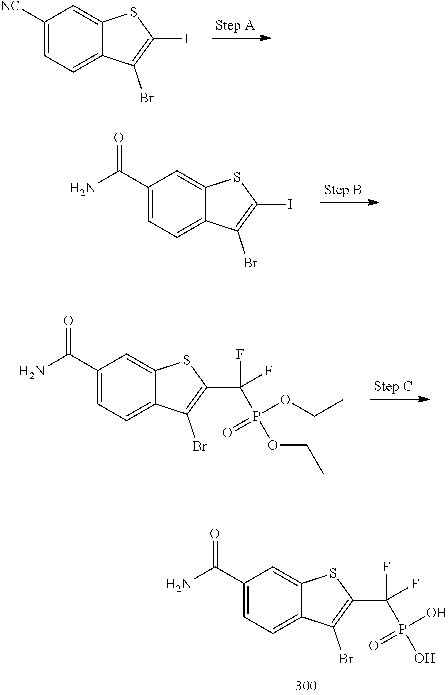 [SMILES: N#Cc1ccc2c(Br)c(I)sc2c1 ; NC(=O)c1ccc2c(Br)c(I)sc2c1 ; CCOP(=O)(OCC)C(F)(F)c1sc2cc(C(N)=O)ccc2c1Br ; NC(=O)c1ccc2c(Br)c(C(F)(F)P(=O)(O)O)sc2c1]

### Step A: Synthesis of 3-bromo-2-iodobenzo[b]thiophene-6-carboxamide

To a mixture of 3-bromo-2-iodobenzo[b]thiophene-6-carbonitrile (210 mg, 0.58 mmol) in EtOH (1 mL), and water (1 mL) was added hydrido(dimethylphosphinous acid-kP)[hydrogen bis(dimethylphosphinito-kP)]platinum(II) (25 mg, 0.058 mmol). The reaction was allowed to stir for 18 hours at 100° C. The reaction was cooled to room temperature, diluted with EtOAc (10 mL), and washed brine (3×10 mL). The organic layer was separated, dried over anhydrous Na₂SO₄, filtered, and concentrated in vacuo to provide 3-bromo-2-iodobenzo[b]thiophene-6-carboxamide, which was used without purification in the next step. MS (ESI, m/z): 382, 384 (M+H)⁺.

### Step B: Synthesis of diethyl ((3-bromo-6-carbamoylbenzo[b]thiophen-2-yl)difluoromethyl)phosphonate

To a mixture of 3-bromo-2-iodobenzo[b]thiophene-6-carboxamide (130 mg, 0.34 mmol), and copper (I) bromide (98 mg, 0.69 mmol) in DMF (3 mL) was added a solution of ((diethoxyphosphoryl)difluoromethyl)zinc (II) bromide (1 M in THF, 1 mL, 1 mmol) under an argon atmosphere. The mixture was allowed to stir at room temperature for 18 hours. The reaction was quenched by addition of 1M HCl (2 mL), and extracted with EtOAc (5 mL). The organic extract was washed with water (3×5 mL), dried over anhydrous Na₂SO₄, filtered, and concentrated in vacuo to provide diethyl ((3-bromo-6-carbamoylbenzo[b]thiophen-2-yl)difluoromethyl)phosphonate, which was used without purification in the next step. MS (ESI, m/z): 442, 444 (M+H)⁺.

### Step C: Synthesis of ((3-bromo-6-carbamoylbenzo[b]thiophen-2-yl)difluoromethyl)phosphonic acid

To a mixture of diethyl ((3-bromo-6-carbamoylbenzo[b]thiophen-2-yl)difluoromethyl)phosphonate (150 mg, 0.34 mmol) in DCM (3 mL) was added TMS-Br (1 mL, 7.7 mmol). The reaction was allowed to stir for 18 hours at 50° C., then concentrated in vacuo, and the resulting residue was diluted with MeOH (1 mL), and again concentrated in vacuo, and the resulting residue was purified using reverse phase HPLC (eluting acetonitrile in water, with 0.1% TFA modifier) to provide ((3-bromo-6-carbamoylbenzo[b]thiophen-2-yl)difluoromethyl)phosphonic acid (compound 300). ¹H NMR (499 MHz, DMSO-d₆) δ 8.62 (s, 1H), 8.16 (s, 1H), 8.06 (d, J=8.5 Hz, 1H), 7.93 (d, J=8.5 Hz, 1H), 7.57 (s, 1H). MS (ESI, m/z): 384, 386 (M−H)⁻.

### Example 39

### Preparation of Compound 301

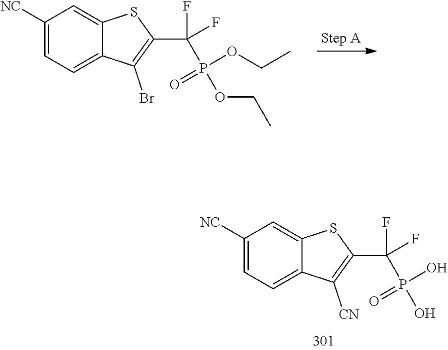 [SMILES: CCOP(=O)(OCC)C(F)(F)c1sc2cc(C#N)ccc2c1Br ; N#Cc1ccc2c(C#N)c(C(F)(F)P(=O)(O)O)sc2c1]

A mixture of diethyl ((3-bromo-6-cyanobenzo[b]thiophen-2-yl)difluoromethyl)phosphonate (34 mg, 0.081 mmol), and copper(I) cyanide (36 mg, 0.40 mmol) in NMP (0.8 mL) was stirred and heated to 150° C. for 3 hours. The mixture was cooled to room temperature. TMS-Br (0.21 mL 1.6 mmol) was added to the mixture and the mixture was stirred and heated to 50° C. for 1 hour. The mixture was cooled to room temperature, and quenched with methanol (0.1 mL), and sodium hydroxide (2N, 0.1 mL). The mixture was purified using reverse phase HPLC (eluting acetonitrile in water, with 0.1% TFA modifier) to provide ((3,6-dicyanobenzo[b]thiophen-2-yl)difluoromethyl)phosphonic acid (compound 301). ¹H NMR (600 MHz, DMSO-d₆) δ 8.89 (s, 1H), 8.15-8.00 (m, 2H). MS (ESI, m/z): 315 (M+H)⁺

### Example 40

### Preparation of Compound 302

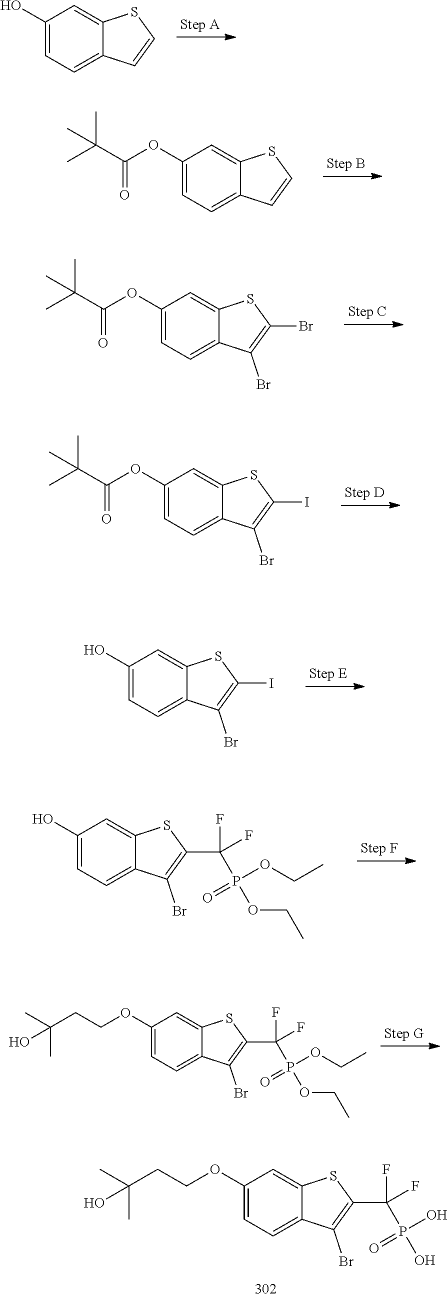

### Step A: Synthesis of benzo[b]thiophen-6-yl pivalate

Pivaloyl chloride (4.80 ml, 39.0 mmol) was added dropwise to a mixture of benzo[b]thiophen-6-ol (4.88 g, 32.5 mmol) in pyridine (100 ml) at 0° C. The mixture was warmed to room temperature and stirred for 72 hours. The mixture was concentrated in vacuo and then partitioned between EtOAc (200 mL), and brine (100 mL). The organic layer was separated, washed with brine (3×50 mL), dried over Na₂SO₄, filtered, and concentrated in vacuo to provide benzo[b]thiophen-6-yl pivalate, which was used without further purification in the next step. ¹H NMR (499 MHz, chloroform-d) δ 7.82 (d, J=8.6 Hz, 1H), 7.62 (s, 1H), 7.44 (d, J=5.4 Hz, 1H), 7.34 (d, J=5.4 Hz, 1H), 7.10 (dd, J=8.6, 1.5 Hz, 1H), 1.42 (s, 9H). MS (ESI, m/z): 235 (M+H)⁺

### Step B: Synthesis of 2,3-dibromobenzo[b]thiophen-6-yl pivalate

Bromine (1.94 mL, 37.6 mmol) was added to a mixture of benzo[b]thiophen-6-yl pivalate (4.2 g, 18 mmol) in DCM (50 mL) at 0° C. and then warmed to room temperature, and stirred overnight. The reaction was quenched with saturated aqueous sodium thiosulfate (100 mL), then diluted with brine (100 mL). The organic layer was separated and the aqueous layer was extracted with additional DCM (100 mL). The combined organic extracts were dried over anhydrous Na₂SO₄, filtered, and concentrated in vacuo to provide 2,3-dibromobenzo[b]thiophen-6-yl pivalate, which was without further purification in the next step. ¹H NMR (499 MHz, DMSO-d₆) δ 7.91 (d, J=2.0 Hz, 1H), 7.75 (d, J=8.7 Hz, 1H), 7.29 (dd, J=8.7, 2.1 Hz, 1H), 1.34 (s, 9H). MS (ESI, m/z): 393 (M+H)⁺

### Step C: Synthesis of 3-bromo-2-iodobenzo[b]thiophen-6-yl pivalate

A solution of isopropylmagnesium chloride lithium chloride complex (1.3 M in THF, 1.2 mL, 1.6 mmol) was added to a mixture of 2,3-dibromobenzo[b]thiophen-6-yl pivalate (505 mg, 1.29 mmol) in THF (5.0 mL) at −50° C. The mixture was allowed to stir at −50° C. for 2 hours. A mixture of iodine (490 mg, 1.93 mmol) in THF (5.0 mL) was added and the mixture was warmed to room temperature, and stirred for 18 hours. The reaction was quenched with saturated aqueous sodium thiosulfate (20 mL) and diluted with EtOAc (50 mL). The organic layer was separated, washed with brine (3×20 mL), dried over anhydrous sodium sulfate, filtered, and concentrated in vacuo to provide 3-bromo-2-iodobenzo[b]thiophen-6-yl pivalate, which was used without purification in the next step. MS (ESI, m/z): 439, 441 (M+H)⁺

### Step D: Synthesis of 3-bromo-2-iodobenzo[b]thiophen-6-ol

A mixture of 50% potassium hydroxide in water (3.0 mL, 27 mmol) was added to a mixture of 3-bromo-2-iodobenzo[b]thiophen-6-yl pivalate (520 mg, 1.18 mmol) in ethanol (10 mL). The mixture was stirred and heated to 100° C. for 18 hours. The mixture was cooled to room temperature, quenched with hydrochloric acid (1.0 M in water, added to pH 2 as determined by litmus test), and extracted with EtOAc (25 mL). The organic layer was separated, washed with brine (3×25 mL), dried over anhydrous Na₂SO₄, filtered, and concentrated in vacuo, and the resulting residue was purified using silica gel chromatography (eluting EtOAc in isohexane). The isolated material was contaminated with pivalic acid which was removed by dissolving the residue in EtOAc (25 mL) and washing with saturated sodium bicarbonate (3×25 mL). The organic layer was separated, dried over anhydrous Na₂SO₄, filtered, and concentrated in vacuo to provide 3-bromo-2-iodobenzo[b]thiophen-6-ol. ¹H NMR (499 MHz, chloroform-d) δ 7.64 (d, J=8.7 Hz, 1H), 7.21 (d, J=2.2 Hz, 1H), 6.95 (dd, J=8.7, 2.3 Hz, 1H).

### Step E: Synthesis of diethyl ((3-bromo-6-hydroxybenzo[b]thiophen-2-yl)difluoromethyl)phosphonate

A mixture of copper (I) bromide (163 mg, 1.14 mmol), and 3-bromo-2-iodobenzo[b]thiophen-6-ol (202 mg, 0.569 mmol) was evacuated and backfilled with argon (3×). DMF (6.0 mL) was added to the mixture followed by a solution of ((diethoxyphosphoryl)difluoromethyl)zinc (II) bromide (1.0 M in THF, 2.0 mL, 2.0 mmol). The mixture was allowed to stir at room temperature for 18 hours. The reaction was quenched with HCl (1.0 M in water, added until pH=2 as determined by litmus test), then diluted with brine (10 mL), and EtOAc (50 mL). The organic layer was separated, washed with brine (3×25 mL), dried over anhydrous sodium sulfate, filtered, and concentrated in vacuo, and the resulting residue was purified using silica gel chromatography (eluting [3:1 EtOAc:EtOH]in hexanes) to provide diethyl ((3-bromo-6-hydroxybenzo[b]thiophen-2-yl)difluoromethyl)phosphonate. ¹H NMR (499 MHz, DMSO-d₆) δ 10.25 (s, 1H), 7.71 (d, J=8.8 Hz, 1H), 7.44 (d, J=2.0 Hz, 1H), 7.09 (dd, J=8.8, 2.1 Hz, 1H), 4.26-4.16 (m, 4H), 1.34-1.22 (m, 6H). MS (ESI, m/z): 415, 417 (M+H)⁺

### Step F: Synthesis of diethyl ((3-bromo-6-(3-hydroxy-3-methylbutoxy)benzo[b]thiophen-2-yl)difluoromethyl)phosphonate

4-bromo-2-methylbutan-2-ol (126 mg, 0.751 mmol) was added to a mixture of diethyl ((3-bromo-6-hydroxybenzo[b]thiophen-2-yl)difluoromethyl)phosphonate (260 mg, 0.626 mmol), and potassium carbonate (173 mg, 1.25 mmol) in DMF (6.0 mL) at room temperature. The reaction was allowed to stir for 18 hours at room temperature. The reaction was quenched with hydrochloric acid (1.0 M in water, 5.0 mL, 5.0 mmol), and extracted with ethyl acetate (20 mL). The organic layer was separated, washed with brine (3×20 mL), dried over anhydrous Na₂SO₄, filtered, and concentrated in vacuo, and the resulting residue was purified using silica gel chromatography (eluting [3:1 EtOAc/EtOH]in hexanes to provide diethyl ((3-bromo-6-(3-hydroxy-3-methylbutoxy)benzo[b]thiophen-2-yl)difluoromethyl)phosphonate.

### Step G: Synthesis of ((3-bromo-6-(3-hydroxy-3-methylbutoxy)benzo[b]thiophen-2-yl)difluoromethyl)phosphonic acid

TMS-Br (0.50 mL, 3.9 mmol) was added to a mixture of diethyl ((3-bromo-6-(3-hydroxy-3-methylbutoxy)benzo[b]thiophen-2-yl)difluoromethyl)phosphonate (72 mg, 0.14 mmol) in DCM (3 mL) at room temperature. The mixture was allowed to stir at room temperature for 18 hours. The mixture was concentrated in vacuo, and the resulting residue was diluted with DCM (3 mL) and concentrated in vacuo. Ammonium hydroxide (500 uL), and MeOH (1.0 mL) were added to the residue, and the mixture was concentrated in vacuo, and the resulting residue was purified using reverse phase HPLC (eluting acetonitrile in water, with 0.1% TFA modifier) to provide ((3-bromo-6-(3-hydroxy-3-methylbutoxy)benzo[b]thiophen-2-yl)difluoromethyl)phosphonic acid (compound 302). ¹H NMR (499 MHz, DMSO-d₆) δ 7.73 (d, J=8.9 Hz, 1H), 7.71 (s, 1H), 7.17 (dd, J=8.9, 2.0 Hz, 1H), 4.18 (t, J=7.2 Hz, 2H), 1.89 (t, J=7.1 Hz, 2H), 1.19 (s, 6H). MS (ESI, m/z): 427, 429 (M+H−H₂O)⁺

### Example 41

### Preparation of Compound 303

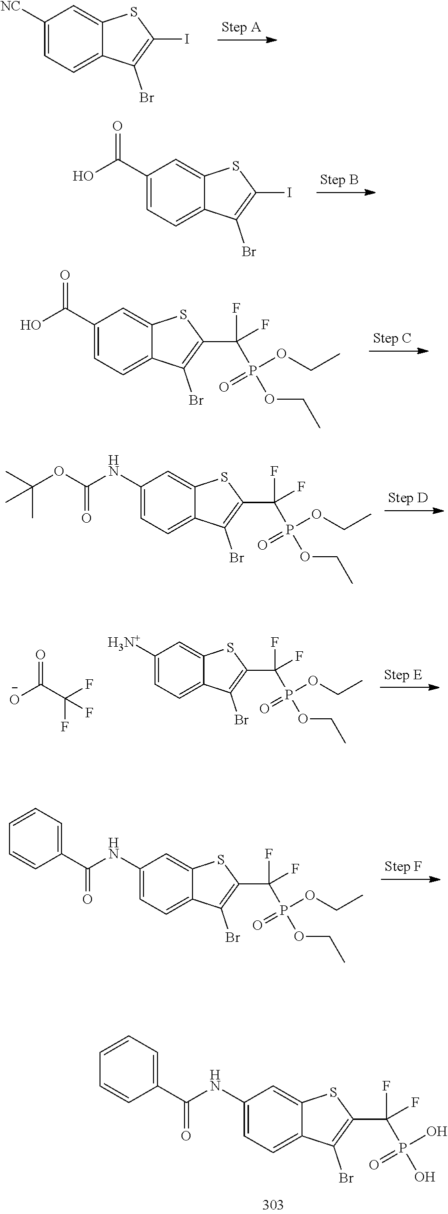

### Step A: Synthesis of 3-bromo-2-iodobenzo[b]thiophene-6-carboxylic acid

3-bromo-2-iodobenzo[b]thiophene-6-carbonitrile (5.8 g, 16 mmol) was suspended in water (20 mL), and acetic acid (20 mL), and cooled to 0° C. Sulfuric acid (20 mL, 375 mmol) was added slowly to the mixture, and the mixture was stirred and heated to 115° C. for 18 hours. The mixture was cooled to room temperature and diluted with water. The mixture was filtered, and the collected solids were washed with water (3×10 mL), azeotroped with toluene (3×25 mL), and dried in vacuo to provide 3-bromo-2-iodobenzo[b]thiophene-6-carboxylic acid. ¹H NMR (499 MHz, DMSO-d₆) δ 8.65 (s, 1H), 8.02-7.99 (m, 1H), 7.80 (d, J=8.4 Hz, 1H). MS (ESI, m/z): 383, 385 (M+H)⁺

### Step B: Synthesis of 3-bromo-2-((diethoxyphosphoryl)difluoromethyl)benzo[b]thiophene-6-carboxylic acid

A mixture of copper (I) bromide (2.70 g, 18.9 mmol), and 3-bromo-2-iodobenzo[b]thiophene-6-carboxylic acid (3.61 g, 9.43 mmol) in DMF (100 mL) was evacuated and backfilled with argon (3×). A solution of ((diethoxyphosphoryl)difluoromethyl)zinc(II) bromide (1.0 M in THF, 28 ml, 28 mmol) was added to the mixture, and the mixture was allowed to stir at room temperature for 18 hours. The reaction was quenched with 1M HCl (50 mL), then diluted with distilled water (200 mL). The mixture was filtered, and the collected solids were washed with saturated ammonium chloride (3×100 mL), distilled water (1×100 mL), and methanol (20 mL). The resulting residue was purified using silica gel chromatography (eluting with EtOAc/EtOH (3:1) in hexanes to provide 3-bromo-2-((diethoxyphosphoryl)difluoromethyl)benzo[b]thiophene-6-carboxylic acid. MS (ESI, m/z): 443, 445 (M+H)⁺

### Step C: Synthesis of tert-butyl (3-bromo-2-((diethoxyphosphoryl)difluoromethyl)benzo[b]thiophen-6-yl)carbamate

A mixture of 3-bromo-2-((diethoxyphosphoryl)difluoromethyl)benzo[b]thiophene-6-carboxylic acid (1.00 g, 2.26 mmol), diphenylphosphoryl azide (535 μl, 2.48 mmol), tert-butanol (2.16 mL, 22.6 mmol), and Hünig's base (500 μl, 2.86 mmol) in dioxane (22 mL) was stirred and heated to 90° C. for 3 hours. The reaction mixture was cooled, quenched with saturated sodium bicarbonate (10 mL), and diluted with DCM (20 mL). The organic layer was separated and concentrated in vacuo, and the resulting residue was purified using silica gel chromatography (eluting with [3:1 EtOAc/EtOH]in hexanes to provide tert-butyl (3-bromo-2-((diethoxyphosphoryl)difluoromethyl)benzo[b]thiophen-6-yl)carbamate. MS (ESI, m/z): 514, 516 (M+H)⁺

### Step D: Synthesis of 3-bromo-2-((diethoxyphosphoryl)difluoromethyl)benzo[b]thiophen-6-aminium 2,2,2-trifluoroacetate

A mixture of tert-butyl (3-bromo-2-((diethoxyphosphoryl)difluoromethyl) benzo[b]thiophen-6-yl)carbamate (588 mg, 1.14 mmol) in DCM (5 mL), and TFA (2.0 mL) was allowed to stir at room temperature for 3 hours. The mixture was concentrated in vacuo to provide 3-bromo-2-((diethoxyphosphoryl)difluoromethyl)benzo[b]thiophen-6-aminium 2,2,2-trifluoroacetate. The isolated product was used without purification in the next step. MS (ESI, m/z): 414, 416 (M+H)⁺

### Step E: Synthesis of diethyl ((6-benzamido-3-bromobenzo[b]thiophen-2-yl)difluoromethyl)phosphonate

Benzoyl chloride (26 μl, 0.23 mmol) was added to a mixture of 3-bromo-2-((diethoxyphosphoryl)difluoromethyl)benzo[b]thiophen-6-aminium 2,2,2-trifluoroacetate (100 mg, 0.189 mmol), DMAP (2.3 mg, 0.019 mmol), and Hünig's base (132 μl, 0.757 mmol) in DCM (2.0 mL). The mixture was allowed to stir at room temperature for 18 hours. The reaction was quenched with saturated aqueous sodium bicarbonate (2 mL), and the organic layer was separated with a phase separator. The filtrate was concentrated in vacuo to provide diethyl ((6-benzamido-3-bromobenzo[b]thiophen-2-yl)difluoromethyl)phosphonate, which was used without purification in the next step.

### Step F: Synthesis of ((6-benzamido-3-bromobenzo[b]thiophen-2-yl)difluoromethyl)phosphonic acid

TMS-Br (500 μl, 3.85 mmol) was added to a mixture of diethyl ((6-benzamido-3-bromobenzo[b]thiophen-2-yl)difluoromethyl)phosphonate (98 mg, 0.19 mmol) in DCM (2.0 mL). The resulting reaction was heated to 50° C. for 18 hours. The mixture was cooled to room temperature, quenched with MeOH (2 mL), then concentrated in vacuo. The resulting residue was purified using reverse phase HPLC (eluting acetonitrile in water, with 0.1% TFA modifier) to provide ((6-benzamido-3-bromobenzo[b]thiophen-2-yl)difluoromethyl)phosphonic acid (compound 303). ¹H NMR (499 MHz, DMSO-d₆) δ 10.61 (s, 1H), 8.66 (s, 1H), 8.01 (d, J=7.2 Hz, 2H), 7.91-7.83 (m, 2H), 7.66-7.60 (m, 1H), 7.57 (t, J=7.3 Hz, 2H). MS (ESI, m/z): 462, 464 (M+H)⁺

### Example 42

### Preparation of Compound 404

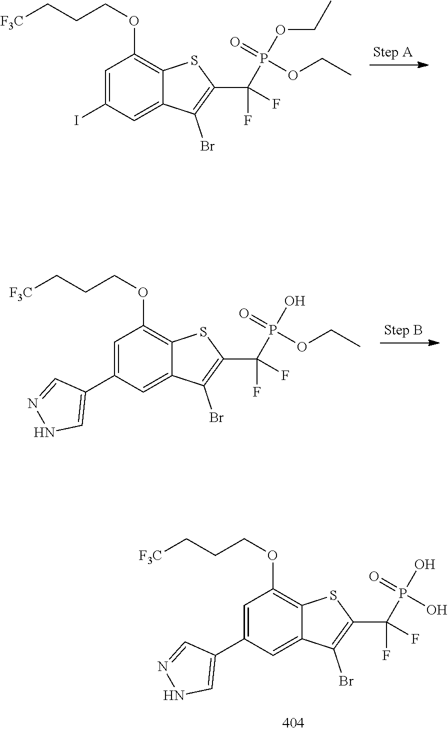 [SMILES: CCOP(=O)(OCC)C(F)(F)c1sc2c(OCCCC(F)(F)F)cc(I)cc2c1Br ; CCOP(=O)(O)C(F)(F)c1sc2c(OCCCC(F)(F)F)cc(-c3cn[nH]c3)cc2c1Br ; O=P(O)(O)C(F)(F)c1sc2c(OCCCC(F)(F)F)cc(-c3cn[nH]c3)cc2c1Br]

### Step A: Synthesis of ethyl hydrogen ((3-bromo-5-(1H-pyrazol-4-yl)-7-(4,4,4-trifluorobutoxy)benzo[b]thiophen-2-yl)difluoromethyl)phosphonate

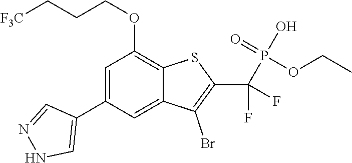 [SMILES: CCOP(=O)(O)C(F)(F)c1sc2c(OCCCC(F)(F)F)cc(-c3cn[nH]c3)cc2c1Br]

A mixture of diethyl ((3-bromo-5-iodo-7-(4,4,4-trifluorobutoxy)benzo[b]thiophen-2-yl) difluoromethyl)phosphonate (60 mg, 0.092 mmol), tert-butyl 4-(4,4,5,5-tetramethyl-1,3,2-dioxaborolan-2-yl)-1H-pyrazole-1-carboxylate (27 mg, 0.092 mmol), potassium phosphate tribasic (39 mg, 0.18 mmol), and dichloro[9,9-dimethyl-4,5-bis(diphenylphosphino)xanthene]palladium (II) (7 mg, 9 μmol) in 1,4-dioxane (6 mL), and water (1.2 mL) was stirred and heated at 90° C. for 16 hours under an argon atmosphere. The reaction mixture was concentrated in vacuo, and the resulting residue was purified using reverse phase HPLC [eluting acetonitrile in (water with 0.01M ammonium bicarbonate)]to provide ethyl hydrogen ((3-bromo-5-(1H-pyrazol-4-yl)-7-(4,4,4-trifluorobutoxy)benzo[b]thiophen-2-yl)difluoromethyl) phosphonate. MS (ESI, m/z): 561, 563 (M−H)⁻

### Step B: Synthesis of((3-bromo-5-(1H-pyrazol-4-yl)-7-(4,4,4-trifluorobutoxy)benzo[b]thiophen-2-yl)difluoromethyl)phosphonic acid

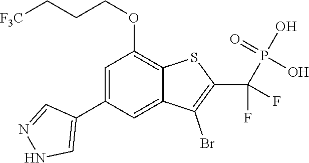 [SMILES: O=P(O)(O)C(F)(F)c1sc2c(OCCCC(F)(F)F)cc(-c3cn[nH]c3)cc2c1Br]

To a stirred solution of ethyl hydrogen ((3-bromo-5-(1H-pyrazol-4-yl)-7-(4,4,4-trifluorobutoxy)benzo[b]thiophen-2-yl)difluoromethyl)phosphonate (20 mg, 0.036 mmol) in DMF (0.4 mL) was added bromotrimethylsilane (138 μL, 1.07 mmol). The resulting reaction was heated to 60° C. and allowed to stir at this temperature for 2 hours. The reaction mixture was quenched with ethanol (0.5 mL), and the resulting solution was directly purified using reverse phase HPLC [eluting acetonitrile in (water with 0.01 M ammonium bicarbonate)] to provide ((3-bromo-5-(1H-pyrazol-4-yl)-7-(4,4,4-trifluorobutoxy)benzo[b]thiophen-2-yl)difluoromethyl)phosphonic acid (Compound 404). ¹H NMR (400 MHz, DMSO-d₆) δ 8.19 (s, 2H), 7.57 (s, 1H), 7.37 (s, 1H), 4.45-4.28 (m, 2H), 2.60-2.41 (m, 2H), 2.13-1.97 (m, 2H). MS (ESI, m/z): 533, 535 (M−H)⁻

### Example 43

### Preparation of Compound 405

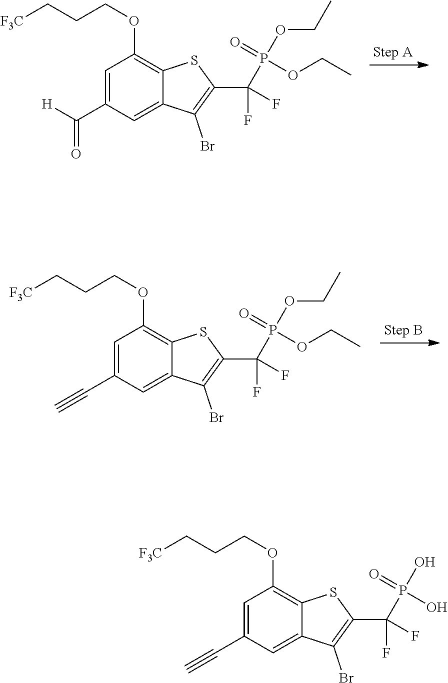 [SMILES: CCOP(=O)(OCC)C(F)(F)c1sc2c(OCCCC(F)(F)F)cc(C=O)cc2c1Br ; C#Cc1cc(OCCCC(F)(F)F)c2sc(C(F)(F)P(=O)(OCC)OCC)c(Br)c2c1 ; C#Cc1cc(OCCCC(F)(F)F)c2sc(C(F)(F)P(=O)(O)O)c(Br)c2c1]

### Step A: Synthesis of diethyl ((3-bromo-5-ethynyl-7-(4,4,4-trifluorobutoxy)benzo[b]thiophen-2-yl)difluoromethyl)phosphonate

To a mixture of diethyl ((3-bromo-5-formyl-7-(4,4,4-trifluorobutoxy)benzo[b]thiophen-2-yl)difluoromethyl)phosphonate (100 mg, 0.181 mmol) in methanol (1 mL) was added dimethyl (1-diazo-2-oxopropyl)phosphonate (52 mg, 0.27 mmol), and potassium carbonate (38 mg, 0.27 mmol) at room temperature under an argon atmosphere. The resulting reaction was allowed to stir for 1 hour at room temperature, then was quenched with water (20 mL) and extracted with ethyl acetate (50 mL). The organic extract was dried over anhydrous sodium sulfate, filtered and concentrated in vacuo. The resulting residue was purified using silica gel chromatography (eluting ethyl acetate in petroleum ether) to provide diethyl((3-bromo-5-ethynyl-7-(4,4,4-trifluorobutoxy)benzo[b]thiophen-2-yl)difluoromethyl)phosphonate. MS (ESI, m/z): 549, 551 (M+H)⁺

### Step B: Synthesis of ((3-bromo-5-ethynyl-7-(4,4,4-trifluorobutoxy)benzo[b]thiophen-2-yl)difluoromethyl)phosphonic acid

To a mixture of diethyl ((3-bromo-5-ethynyl-7-(4,4,4-trifluorobutoxy)benzo[b]thiophen-2-yl)difluoromethyl)phosphonate (15 mg, 0.027 mmol) in DMF (0.2 mL) was added dropwise bromotrimethylsilane (0.11 mL, 0.819 mmol) at room temperature under an argon atmosphere. The resulting reaction was heated to 60° C. and allowed to stir at this temperature for 1 hour. The reaction was then quenched with ethanol (0.5 mL) and concentrated in vacuo. The resulting residue was purified using reverse phase HPLC [eluting acetonitrile in (water with 0.01 M ammonium bicarbonate)]to provide ((3-bromo-5-ethynyl-7-(4,4,4-trifluorobutoxy)benzo[b]thiophen-2-yl)difluoromethyl)phosphonic acid (Compound 405). ¹H NMR (400 MHz, D₂O) δ 7.74 (s, 1H), 7.14 (s, 1H), 4.35 (t, J=6.2 Hz, 2H), 3.62 (s, 1H), 2.57-2.43 (m, 2H), 2.24-2.15 (m, 2H). MS (ESI, m/z): 491, 493 (M−H)⁻

### Example 44

### Preparation of Compound 406

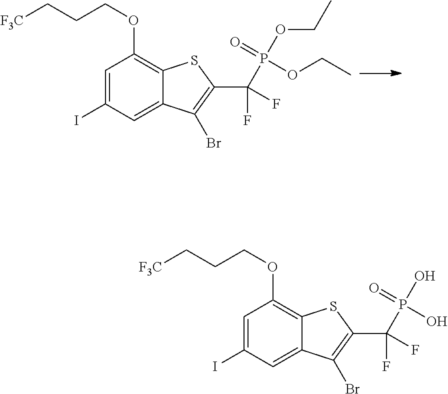 [SMILES: CCOP(=O)(OCC)C(F)(F)c1sc2c(OCCCC(F)(F)F)cc(I)cc2c1Br ; O=P(O)(O)C(F)(F)c1sc2c(OCCCC(F)(F)F)cc(I)cc2c1Br]

To a stirred solution of ethyl hydrogen ((3-bromo-5-iodo-7-(4,4,4-trifluorobutoxy)benzo[b]thiophen-2-yl)difluoromethyl)phosphonate (20 mg, 0.032 mmol) in DMF (250 μL) was added bromotrimethylsilane (125 μL, 0.963 mmol) at room temperature under an argon atmosphere. The resulting reaction was heated to 60° C. for 2 hours. The reaction was quenched with ethanol (0.5 mL), and purified using reverse phase HPLC [eluting acetonitrile in (water with 0.01 M ammonium bicarbonate)]to provide ((3-bromo-5-iodo-7-(4,4,4-trifluorobutoxy)benzo[b]thiophen-2-yl)difluoromethyl)phosphonic acid (Compound 406). ¹H NMR (400 MHz, D₂O) δ 7.98 (s, 1H), 7.37 (s, 1H), 4.35 (d, J=6.6 Hz, 2H), 2.58-2.41 (m, 2H), 2.27-2.11 (m, 2H). MS (ESI, m/z): 593, 595 (M−H)⁻

The following illustrative compounds were made using the methods described in the Example immediately above, and substituting the appropriate reactants and/or reagents.

| Com- | | Mass |
|---|---|---|
| pound | Structure | [M + H]⁺ |
| 407 | 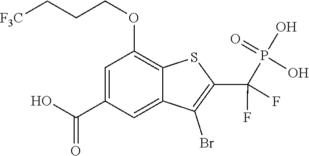 [SMILES: O=C(O)c1cc(OCCCC(F)(F)F)c2sc(C(F)(F)P(=O)(O)O)c(Br)c2c1] | 511, 513 [M − H]⁻ |
| 408 | 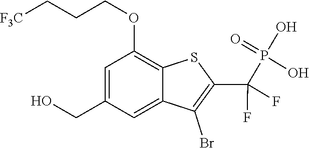 [SMILES: O=P(O)(O)C(F)(F)c1sc2c(OCCCC(F)(F)F)cc(CO)cc2c1Br] | 497, 499 [M − H]⁻ |
| 409 | 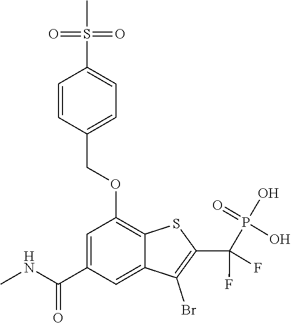 [SMILES: CNC(=O)c1cc(OCc2ccc(S(C)(=O)=O)cc2)c2sc(C(F)(F)P(=O)(O)O)c(Br)c2c1] | 584, 586 |

### Example 45

### Preparation of Compound 410

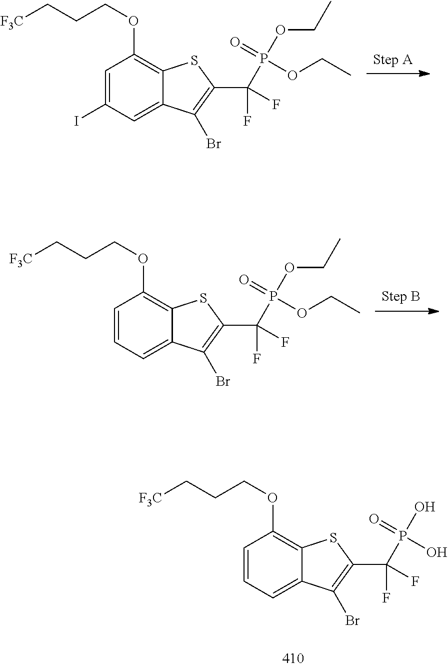 [SMILES: CCOP(=O)(OCC)C(F)(F)c1sc2c(OCCCC(F)(F)F)cc(I)cc2c1Br ; CCOP(=O)(OCC)C(F)(F)c1sc2c(OCCCC(F)(F)F)cccc2c1Br ; O=P(O)(O)C(F)(F)c1sc2c(OCCCC(F)(F)F)cccc2c1Br]

### Step A: Synthesis of diethyl ((3-bromo-7-(4,4,4-trifluorobutoxy)benzo[b]thiophen-2-yl)difluoromethyl)phosphonate

To a solution of diethyl ((3-bromo-5-iodo-7-(4,4,4-trifluorobutoxy)benzo[b]thiophen-2-yl)difluoromethyl)phosphonate (25 mg, 0.038 mmol) in MeOH (1 mL) was added palladium on carbon (10%, 5 mg). The reaction was put under a hydrogen atmosphere and allowed to stir for 2 hours at room temperature. The reaction mixture was filtered, and concentrated in vacuo to provide diethyl ((3-bromo-7-(4,4,4-trifluorobutoxy)benzo[b]thiophen-2-yl)difluoromethyl)phosphonate, which was used without purification in the next step. ¹H NMR (400 MHz, CDCl₃) δ 7.57-7.53 (m, 1H), 7.47-7.42 (m, 1H), 6.88 (d, J=7.8 Hz, 1H), 4.38-4.20 (m, 6H), 2.45-2.31 (m, 2H), 2.21-2.10 (m, 2H), 1.38 (t, J=7.1 Hz, 6H). MS (ESI, m/z): 525, 527 (M+H)⁺

### Step B: Synthesis of ((3-bromo-7-(4,4,4-trifluorobutoxy)benzo[b]thiophen-2-yl)difluoromethyl)phosphonic acid

To a stirred solution of diethyl ((3-bromo-7-(4,4,4-trifluorobutoxy)benzo[b]thiophen-2-yl)difluoromethyl)phosphonate (20 mg, 0.038 mmol) in DMF (300 μL) was added bromotrimethylsilane (148 μL, 1.14 mmol). The reaction mixture was heated to 60° C. and allowed to stir at this temperature for 2 hours. The reaction was quenched with ethanol (0.5 mL), and the reaction mixture was concentrated in vacuo. The resulting residue was purified using reverse phase HPLC [eluting acetonitrile in (water with 0.01 M ammonium bicarbonate)]to provide ((3-bromo-7-(4,4,4-trifluorobutoxy)benzo[b]thiophen-2-yl)difluoromethyl)phosphonic acid (Compound 410). ¹H NMR (400 MHz, D₂O) δ 7.56 (d, J=8.3 Hz, 1H), 7.53-7.45 (m, 1H), 7.05 (d, J=7.7 Hz, 1H), 4.32 (t, J=6.2 Hz, 2H), 2.52-2.34 (m, 2H), 2.19-2.06 (m, 2H). MS (ESI, m/z): 467, 469 (M−H)⁻

### Example 46

### Preparation of Compound 411

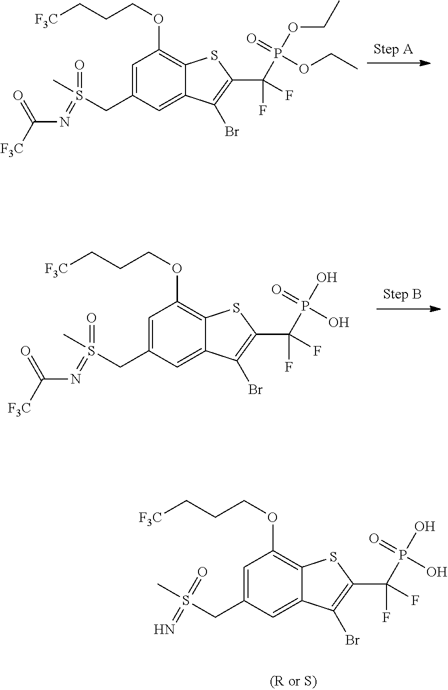 [SMILES: CCOP(=O)(OCC)C(F)(F)c1sc2c(OCCCC(F)(F)F)cc(CS(C)(=O)=NC(=O)C(F)(F)F)cc2c1Br ; CS(=O)(Cc1cc(OCCCC(F)(F)F)c2sc(C(F)(F)P(=O)(O)O)c(Br)c2c1)=NC(=O)C(F)(F)F ; CS(=N)(=O)Cc1cc(OCCCC(F)(F)F)c2sc(C(F)(F)P(=O)(O)O)c(Br)c2c1]

### Step A: Synthesis of ((3-bromo-5-(((R or S)-methyl-N-(2,2,2-trifluoroacetyl)sulfonimidoyl)methyl)-7-(4,4,4-trifluorobutoxy)benzo[b]thiophen-2-yl)difluoromethyl)phosphonic acid

To a mixture of diethyl ((3-bromo-5-(((R or S)-methyl-N-(2,2,2-trifluoroacetyl)sulfonimidoyl)methyl)-7-(4,4,4-trifluorobutoxy)benzo[b]thiophen-2-yl)difluoromethyl)phosphonate (35 mg, 0.049 mmol) in DMF (0.4 mL) was added bromotrimethylsilane (0.191 mL, 1.47 mmol). The resulting reaction was heated to 60° C., and allowed to stir at this temperature for 1.5 hours. The reaction was then quenched with ethanol (2 mL), and concentrated in vacuo to provide ((3-bromo-5-(((R or S)-methyl-N-(2,2,2-trifluoroacetyl)sulfonimidoyl)methyl)-7-(4,4,4-trifluorobutoxy)benzo[b]thiophen-2-yl)difluoromethyl)phosphonic acid, which was used in the next step without purification. MS (ESI, m/z): 654, 656 [M−H]⁻

### Step B: Synthesis of ((3-bromo-5-(((R or S)-methylsulfonimidoyl)methyl)-7-(4,4,4-trifluorobutoxy)benzo[b]thiophen-2-yl)difluoromethyl)phosphonic acid

To a mixture of ((3-bromo-5-(((R or S)-methyl-N-(2,2,2-trifluoroacetyl)sulfonimidoyl)methyl)-7-(4,4,4-trifluorobutoxy)benzo[b]thiophen-2-yl)difluoromethyl)phosphonic acid (35 mg, 0.053 mmol) in methanol (0.4 mL) was added potassium carbonate (37 mg, 0.27 mmol). The resulting reaction was allowed to stir for 16 hours at room temperature, then the reaction mixture was filtered, and the filtrate was directly purified using reverse phase HPLC [eluting acetonitrile in (water with 0.01 M ammonium bicarbonate)]to provide ((3-bromo-5-(((R or S)-methylsulfonimidoyl)methyl)-7-(4,4,4-trifluorobutoxy)benzo[b]thiophen-2-yl)difluoromethyl)phosphonic acid (Compound 411). ¹H NMR (300 MHz, D₂O) δ 7.35 (s, 1H), 6.87 (s, 1H), 4.62 (s, 2H), 4.38-4.20 (m, 2H), 3.00 (s, 3H), 2.53-2.33 (m, 2H), 2.20-2.03 (m, 2H). MS (ESI, m/z): 558, 560 [M−H]⁻

The following illustrative compounds were made using the methods described in the Example immediately above, and substituting the appropriate reactants and/or reagents.

| Com- | | Mass |
|---|---|---|
| pound | Structure | [M + H]⁺ |
| 412 | 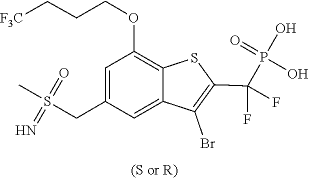 [SMILES: CS(=N)(=O)Cc1cc(OCCCC(F)(F)F)c2sc(C(F)(F)P(=O)(O)O)c(Br)c2c1] | 560, 562 |
| 413 | 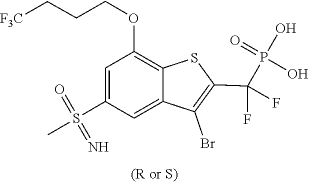 [SMILES: CS(=N)(=O)c1cc(OCCCC(F)(F)F)c2sc(C(F)(F)P(=O)(O)O)c(Br)c2c1] | 546, 548 |
| 414 | 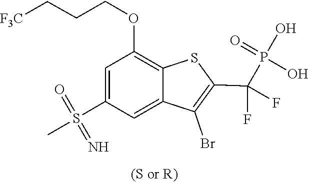 [SMILES: CS(=N)(=O)c1cc(OCCCC(F)(F)F)c2sc(C(F)(F)P(=O)(O)O)c(Br)c2c1] | 546, 548 |

### Example 47

### Preparation of Compound 415

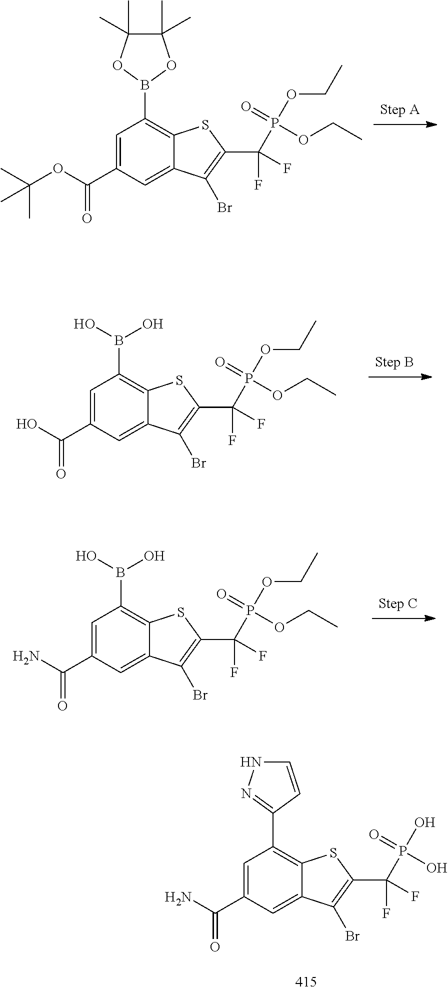 [SMILES: CCOP(=O)(OCC)C(F)(F)c1sc2c(B3OC(C)(C)C(C)(C)O3)cc(C(=O)OC(C)(C)C)cc2c1Br ; CCOP(=O)(OCC)C(F)(F)c1sc2c(B(O)O)cc(C(=O)O)cc2c1Br ; CCOP(=O)(OCC)C(F)(F)c1sc2c(B(O)O)cc(C(N)=O)cc2c1Br ; NC(=O)c1cc(-c2cc[nH]n2)c2sc(C(F)(F)P(=O)(O)O)c(Br)c2c1]

### Step A: Synthesis of 3-bromo-2-((diethoxyphosphoryl)difluoromethyl)-7-(4,4,5,5-tetramethyl-1,3,2-dioxaborolan-2-yl)benzo[b]thiophene-5-carboxylic acid

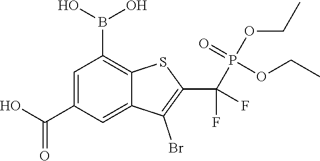 [SMILES: CCOP(=O)(OCC)C(F)(F)c1sc2c(B(O)O)cc(C(=O)O)cc2c1Br]

TFA (10 ml, 130 mmol) was added to a solution of tert-butyl 3-bromo-2-((diethoxyphosphoryl)difluoromethyl)-7-(4,4,5,5-tetramethyl-1,3,2-dioxaborolan-2-yl)benzo[b]thiophene-5-carboxylate (1.13 g, 1.81 mmol) in DCM (20 ml) at room temperature and the resulting reaction was allowed to stir for 2.5 hours. The reaction mixture was then concentrated in vacuo to provide 3-bromo-2-((diethoxyphosphoryl)difluoromethyl)-7-(4,4,5,5-tetramethyl-1,3,2-dioxaborolan-2-yl)benzo[b]thiophene-5-carboxylic acid, which was used without purification. MS (ESI, m/z): 486, 488 (M+H)⁺

### Step B: Synthesis of (3-bromo-5-carbamoyl-2-((diethoxyphosphoryl)difluoromethyl)benzo[b]thiophen-7-yl)boronic acid

A mixture of HATU (1.37 g, 3.60 mmol), N-ethyl-N-isopropylpropan-2-amine (1.22 mL, 7.20 mmol), 3-bromo-2-((diethoxyphosphoryl)difluoromethyl)-7-(4,4,5,5-tetramethyl-1,3,2-dioxaborolan-2-yl)benzo[b]thiophene-5-carboxylic acid (1.02 g, 1.8 mmol), and ammonium chloride (193 mg, 3.60 mmol) in DMF (9 mL) was allowed to stir at room temperature for 1.5 hours. The reaction mixture was partially concentrated in vacuo, and the concentrate was diluted with EtOAc (100 mL), and washed with brine (3×15 mL). The organic phase was collected, dried over anhydrous sodium sulfate, filtered, and concentrated in vacuo. The resulting residue was purified using reverse phase HPLC (eluting acetonitrile in water, with 0.1% TFA) to provide (3-bromo-5-carbamoyl-2-((diethoxyphosphoryl)difluoromethyl)benzo[b]thiophen-7-yl)boronic acid. MS (ESI, m/z): 485, 487 (M+H)⁺

### Step C: Synthesis of ((3-bromo-5-carbamoyl-7-(1H-pyrazol-3-yl)benzo[b]thiophen-2-yl)difluoromethyl)phosphonic acid

A mixture of (3-bromo-5-carbamoyl-2-((diethoxyphosphoryl)difluoromethyl)benzo[b]thiophen-7-yl)boronic acid (45 mg, 0.093 mmol), dichloro[9,9-dimethyl-4,5-bis(diphenylphosphino) xanthene]palladium (II) (7 mg, 9 μmol), 1-tert-butoxycarbonyl-3-iodo-1H-pyrazole (27 mg, 0.093 mmol), and potassium phosphate tribasic (39 mg, 0.19 mmol) in 1,4-dioxane:water (5:1) was heated to 90° C., and allowed to stir at this temperature for 18 hours. The reaction mixture was cooled to room temperature, concentrated in vacuo, and the resulting residue was taken up in DMSO (1.5 mL), and filtered. The filtrate was treated with TMSBr (400 μl, 3.03 mmol), and the resulting reaction was heated to 60° C., and allowed to stir at this temperature for 1.5 hours. The reaction mixture was then quenched with MeOH (0.5 mL), and concentrated in vacuo. The resulting residue was purified using reverse phase HPLC (eluting acetonitrile in water, with TFA modifier) to provide ((3-bromo-5-carbamoyl-7-(1H-pyrazol-3-yl)benzo[b]thiophen-2-yl)difluoromethyl)phosphonic acid (Compound 415). ¹H NMR (499 MHz, DMSO-d₆) δ 8.50 (s, 1H), 8.39 (s, 1H), 8.37 (s, 1H), 7.99 (d, J=2.2 Hz, 1H), 7.63 (s, 1H), 7.10 (d, J=2.2 Hz, 1H). MS (ESI, m/z) 451, 453 (M+H)⁺

### Example 48

### Preparation of Compound 416

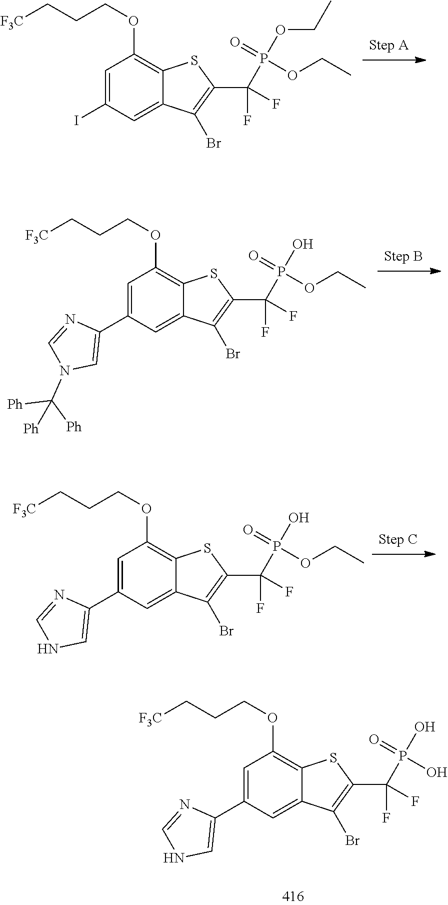 [SMILES: CCOP(=O)(OCC)C(F)(F)c1sc2c(OCCCC(F)(F)F)cc(I)cc2c1Br ; CCOP(=O)(O)C(F)(F)c1sc2c(OCCCC(F)(F)F)cc(-c3cn(C(c4ccccc4)(c4ccccc4)c4ccccc4)cn3)cc2c1Br ; CCOP(=O)(O)C(F)(F)c1sc2c(OCCCC(F)(F)F)cc(-c3c[nH]cn3)cc2c1Br ; O=P(O)(O)C(F)(F)c1sc2c(OCCCC(F)(F)F)cc(-c3c[nH]cn3)cc2c1Br]

### Step A: Synthesis of ethyl hydrogen ((3-bromo-7-(4,4,4-trifluorobutoxy)-5-(1-trityl-1H-imidazol-4-yl)benzo[b]thiophen-2-yl)difluoromethyl)phosphonate

A mixture of diethyl ((3-bromo-5-iodo-7-(4,4,4-trifluorobutoxy)benzo[b]thiophen-2-yl)difluoromethyl)phosphonate (100 mg, 0.154 mmol), (1-trityl-1H-imidazol-4-yl)boronic acid (54 mg, 0.15 mmol), dichloro[9,9-dimethyl-4,5-bis(diphenylphosphino)xanthene]palladium (II) (12 mg, 0.015 mmol), and potassium phosphate (65 mg, 0.31 mmol) in 1,4-dioxane (10 mL), and water (2 mL) was heated to 90° C., and allowed to stir at this temperature for 2 hours under an argon atmosphere. The reaction mixture was cooled to room temperature, then directly purified using reverse phase HPLC (eluting acetonitrile in water) to provide ethyl hydrogen ((3-bromo-7-(4,4,4-trifluorobutoxy)-5-(1-trityl-1H-imidazol-4-yl)benzo[b]thiophen-2-yl)difluoromethyl)phosphonate. MS (ESI, m/z): 805, 807 (M+H)⁺

### Step B: Synthesis of ethyl hydrogen ((3-bromo-5-(1H-imidazol-4-yl)-7-(4,4,4-trifluorobutoxy)benzo[b]thiophen-2-yl)difluoromethyl)phosphonate

A mixture of ethyl hydrogen ((3-bromo-7-(4,4,4-trifluorobutoxy)-5-(1-trityl-1H-imidazol-4-yl)benzo[b]thiophen-2-yl)difluoromethyl)phosphonate (23 mg, 0.029 mmol) in hydrogen chloride (12 M in dioxane, 0.20 mL, 2.4 mmol) was heated to 60° C., and allowed to stir at this temperature for 1 hour. The reaction mixture was concentrated in vacuo to provide ethyl hydrogen ((3-bromo-5-(1H-imidazol-4-yl)-7-(4,4,4-trifluorobutoxy)benzo[b]thiophen-2-yl)difluoromethyl)phosphonate, which was used without purification. MS (ESI, m/z): 563, 565 (M+H)⁺

### Step C: Synthesis of ((3-bromo-5-(1H-imidazol-4-yl)-7-(4,4,4-trifluorobutoxy)benzo[b]thiophen-2-yl)difluoromethyl)phosphonic acid

To a mixture of ethyl hydrogen ((3-bromo-5-(1H-imidazol-4-yl)-7-(4,4,4-trifluorobutoxy)benzo[b]thiophen-2-yl)difluoromethyl)phosphonate (16 mg, 0.028 mmol) in DMF (0.2 mL) was added bromotrimethylsilane (120 μL, 0.909 mmol). The resulting reaction was heated to 60° C., and allowed to stir at this temperature for 1.5 hours. The reaction was quenched with ethanol (0.3 mL), concentrated in vacuo, and the resulting residue was purified using reverse phase HPLC [eluting acetonitrile in (water with 0.01 M ammonium bicarbonate)]to provide ((3-bromo-5-(1H-imidazol-4-yl)-7-(4,4,4-trifluorobutoxy)benzo[b]thiophen-2-yl)difluoromethyl)phosphonic acid (Compound 416). ¹H NMR (400 MHz, CD₃₀D) δ 7.80 (s, 1H), 7.78 (s, 1H), 7.52 (s, 1H), 7.32 (s, 1H), 4.33 (t, J=6.1 Hz, 2H), 2.50-2.36 (m, 2H), 2.20-2.08 (m, 2H). MS (ESI, m/z): 535, 537 (M+H)⁺

### Example 49

### Preparation of Compound 417

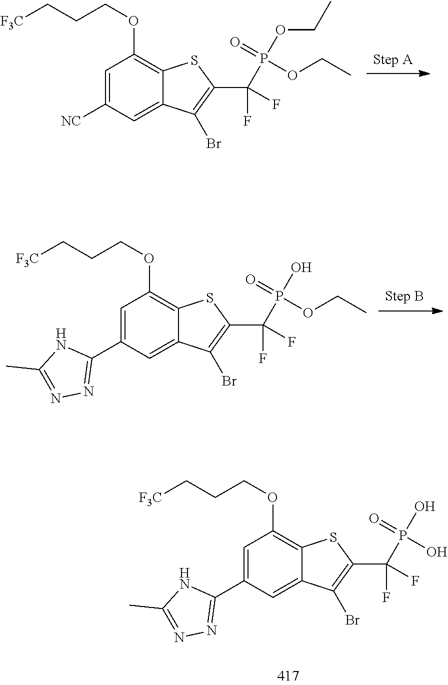

### Step A: Synthesis of ethyl hydrogen ((3-bromo-5-(5-methyl-4H-1,2,4-triazol-3-yl)-7-(4,4,4-trifluorobutoxy)benzo[b]thiophen-2-yl)difluoromethyl)phosphonate

A mixture of diethyl ((3-bromo-5-cyano-7-(4,4,4-trifluorobutoxy)benzo[b]thiophen-2-yl)difluoromethyl)phosphonate (100 mg, 0.182 mmol), cesium carbonate (53 mg, 0.16 mmol), copper (I) bromide (13 mg, 0.091 mmol), and acetimidamide hydrochloride (26 mg, 0.27 mmol) in DMSO (1 mL) was heated to 120° C., and allowed to stir at this temperature for 2 hours. The reaction mixture was then directly purified using reverse phase HPLC (eluting acetonitrile in water, with 0.1% TFA) to provide ethyl hydrogen ((3-bromo-5-(5-methyl-4H-1,2,4-triazol-3-yl)-7-(4,4,4-trifluorobutoxy)benzo[b]thiophen-2-yl)difluoromethyl)phosphonate. MS (ESI, m/z) 578, 580 (M+H)⁺.

### Step B: Synthesis of ((3-bromo-5-(5-methyl-4H-1,2,4-triazol-3-yl)-7-(4,4,4-trifluorobutoxy)benzo[b]thiophen-2-yl)difluoromethyl)phosphonic acid

To a mixture of ethyl hydrogen ((3-bromo-5-(5-methyl-4H-1,2,4-triazol-3-yl)-7-(4,4,4-trifluorobutoxy)benzo[b]thiophen-2-yl)difluoromethyl)phosphonate (4 mg, 7 μmol) in DMF (0.05 mL) was added bromotrimethylsilane (0.027 mL, 0.21 mmol). The resulting reaction was heated to 60° C., and allowed to stir at this temperature for 1 hour. The reaction mixture was quenched with ethanol (0.1 mL), concentrated in vacuo, and the resulting residue was purified using reverse phase HPLC [eluting acetonitrile in (water with 0.01 M ammonium bicarbonate)]to provide ((3-bromo-5-(5-methyl-4H-1,2,4-triazol-3-yl)-7-(4,4,4-trifluorobutoxy)benzo[b]thiophen-2-yl)difluoromethyl)phosphonic acid (Compound 417). ¹H NMR: (400 MHz, DMSO-d₆) δ 8.05 (s, 1H), 7.60 (s, 1H), 4.40-4.26 (m, 2H), 2.42 (s, 3H), 2.55-2.47 (m, 2H), 2.11-1.98 (m, 2H). MS (ESI, m/z) 550, 552 (M+H)⁺

The following illustrative compounds were made using the methods described in the Example immediately above, and substituting the appropriate reactants and/or reagents.

| | | Mass |
|---|---|---|
| Com- | | [M + |
| pound | Structure | H]⁺ |
| 418 | 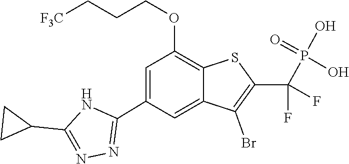 [SMILES: O=P(O)(O)C(F)(F)c1sc2c(OCCCC(F)(F)F)cc(-c3nnc(C4CC4)[nH]3)cc2c1Br] | 576, 578 |

### Example 50

### Preparation of Compound 419

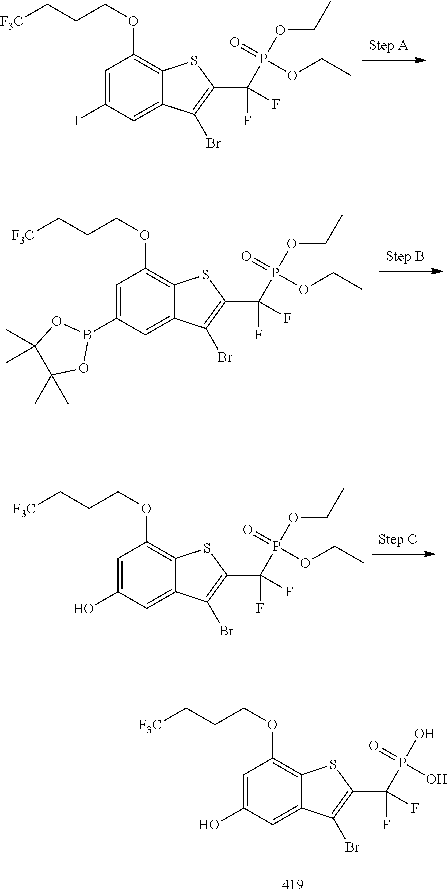

### Step A: Synthesis of diethyl ((3-bromo-5-(4,4,5,5-tetramethyl-1,3,2-dioxaborolan-2-yl)-7-(4,4,4-trifluorobutoxy)benzo[b]thiophen-2-yl)difluoromethyl)phosphonate

A mixture of diethyl ((3-bromo-5-iodo-7-(4,4,4-trifluorobutoxy)benzo[b]thiophen-2-yl)difluoromethyl)phosphonate (150 mg, 0.23 mmol), bis(pinacolato)diboron (88 mg, 0.35 mmol), potassium acetate (45 mg, 0.46 mmol), and dichlorobis(triphenylphosphine)palladium (II) (16 mg, 0.023 mmol) in 1,4-dioxane (15 mL) was heated to 90° C., and allowed to stir at this temperature for 16 hours under an argon atmosphere. The reaction mixture was concentrated in vacuo, and the resulting residue was purified using reverse phase HPLC (eluting acetonitrile in water) to provide diethyl ((3-bromo-5-(4,4,5,5-tetramethyl-1,3,2-dioxaborolan-2-yl)-7-(4,4,4-trifluorobutoxy)benzo[b]thiophen-2-yl)difluoromethyl)phosphonate. MS (ESI, m/z) 651, 653 (M+H)⁺.

### Step B: Synthesis of diethyl ((3-bromo-5-hydroxy-7-(4,4,4-trifluorobutoxy)benzo[b]thiophen-2-yl)difluoromethyl)phosphonate

3-Chloroperoxybenzoic acid (5 mg, 0.03 mmol) was added to mixture of diethyl ((3-bromo-5-(4,4,5,5-tetramethyl-1,3,2-dioxaborolan-2-yl)-7-(4,4,4-trifluorobutoxy)benzo[b]thiophen-2-yl)difluoromethyl)phosphonate (50 mg, 0.028 mmol) in ethanol (400 μL), and water (200 μL). The resulting reaction was allowed to stir at room temperature for 16 hours, then the reaction mixture was quenched with saturated aqueous Na₂S₂O₃ (0.5 mL), and directly purified using reverse phase HPLC (eluting acetonitrile in water) to provide diethyl ((3-bromo-5-hydroxy-7-(4,4,4-trifluorobutoxy)benzo[b]thiophen-2-yl)difluoromethyl)phosphonate. MS (ESI, m/z) 539, 541 (M−H)⁻

### Step C: Synthesis of ((3-bromo-5-hydroxy-7-(4,4,4-trifluorobutoxy)benzo[b]thiophen-2-yl)difluoromethyl)phosphonic acid

To a mixture of diethyl ((3-bromo-5-hydroxy-7-(4,4,4-trifluorobutoxy)benzo[b]thiophen-2-yl)difluoromethyl)phosphonate (4 mg, 7 μmol) in DMF (60 μL) was added bromotrimethylsilane (29 μL, 0.22 mmol). The resulting reaction was heated to 60° C., and allowed to stir at this temperature for 2 hours. The reaction was quenched with ethanol (0.5 mL), concentrated in vacuo, and the resulting residue was purified using reverse phase HPLC [eluting acetonitrile in (water with 0.01 M ammonium bicarbonate)]to provide ammonium ((3-bromo-5-hydroxy-7-(4,4,4-trifluorobutoxy)benzo[b]thiophen-2-yl)difluoromethyl)phosphonic acid (Compound 419). ¹H NMR (400 MHz, D₂O) δ 6.86 (s, 1H), 6.55 (s, 1H), 4.23 (t, J=6.1 Hz, 2H), 2.54-2.32 (m, 2H), 2.16-2.03 (m, 2H). MS (ESI, m/z) 483, 485 (M−H)⁻

### Example 51

### Preparation of Compound 420

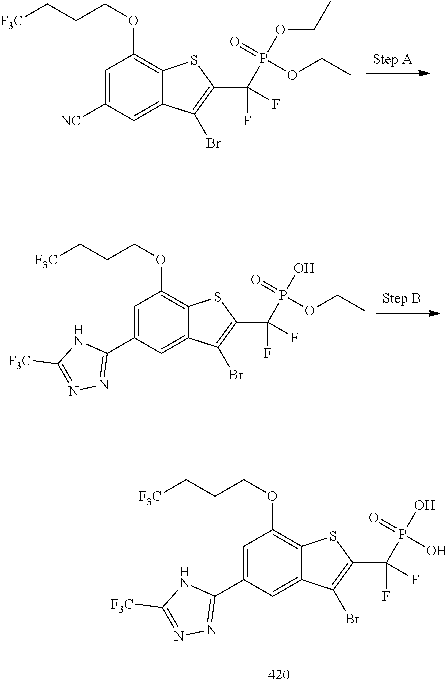 [SMILES: CCOP(=O)(OCC)C(F)(F)c1sc2c(OCCCC(F)(F)F)cc(C#N)cc2c1Br ; CCOP(=O)(O)C(F)(F)c1sc2c(OCCCC(F)(F)F)cc(-c3nnc(C(F)(F)F)[nH]3)cc2c1Br ; O=P(O)(O)C(F)(F)c1sc2c(OCCCC(F)(F)F)cc(-c3nnc(C(F)(F)F)[nH]3)cc2c1Br]

### Step A: Synthesis of ethyl hydrogen ((3-bromo-7-(4,4,4-trifluorobutoxy)-5-(5-(trifluoromethyl)-4H-1,2,4-triazol-3-yl)benzo[b]thiophen-2-yl)difluoromethyl)phosphonate

A mixture of diethyl ((3-bromo-5-cyano-7-(4,4,4-trifluorobutoxy)benzo[b]thiophen-2-yl)difluoromethyl)phosphonate (50 mg, 0.091 mmol), potassium carbonate (13 mg, 0.091 mmol), and 2,2,2-trifluoroacetohydrazide (12 mg, 0.091 mmol) in n-butanol (0.5 mL) was heated to at 150° C., and irradiated with microwave radiation for 4 hours. The reaction mixture was directly purified using reverse phase HPLC [eluting acetonitrile in (water with 0.01 M ammonium bicarbonate)]to provide ethyl hydrogen((3-bromo-7-(4,4,4-trifluorobutoxy)-5-(5-(trifluoromethyl)-4H-1,2,4-triazol-3-yl)benzo[b]thiophen-2-yl)difluoromethyl)phosphonate. MS (ESI, m/z) 630, 632 (M−H)⁻

### Step B: Synthesis of ((3-bromo-7-(4,4,4-trifluorobutoxy)-5-(5-(trifluoromethyl)-4H-1,2,4-triazol-3-yl)benzo[b]thiophen-2-yl)difluoromethyl)phosphonic acid

To a mixture of ethyl hydrogen ((3-bromo-7-(4,4,4-trifluorobutoxy)-5-(5-(trifluoromethyl)-4H-1,2,4-triazol-3-yl)benzo[b]thiophen-2-yl)difluoromethyl)phosphonate (4 mg, 6 μmol) in DMF (0.5 mL) was added bromotrimethylsilane (0.023 mL, 0.18 mmol). The resulting reaction was heated to 60° C., and allowed to stir at this temperature for 1 hour. The reaction was quenched with ethanol (0.1 mL), and concentrated in vacuo. The resulting residue was purified using reverse phase HPLC [eluting acetonitrile in (water with 0.01 M ammonium bicarbonate)]to provide ((3-bromo-7-(4,4,4-trifluorobutoxy)-5-(5-(trifluoromethyl)-4H-1,2,4-triazol-3-yl)benzo[b]thiophen-2-yl)difluoromethyl)phosphonic acid (Compound 420). ¹H NMR (300 MHz, D₂O) ¹H NMR: (300 MHz, D₂O) δ 7.53 (s, 1H), 6.98 (s, 1H), 4.24-4.03 (m, 2H), 2.59-2.27 (m, 2H), 2.21-1.96 (m, 2H). MS (ESI, m/z) 602, 604 (M−H)⁻

### Example 52

### Preparation of Compound 421

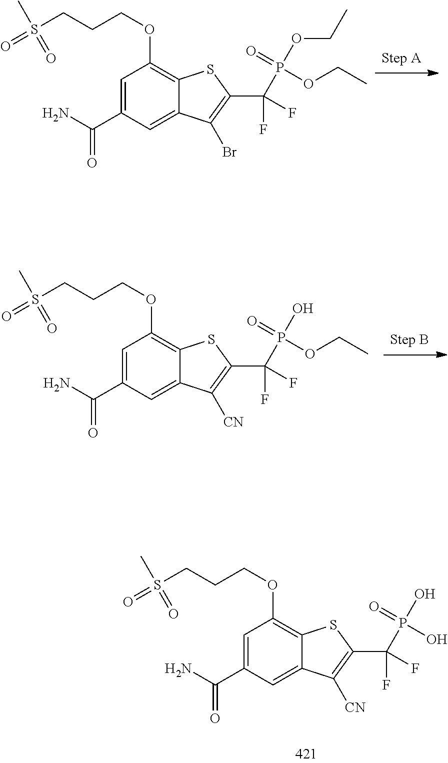 [SMILES: CCOP(=O)(OCC)C(F)(F)c1sc2c(OCCCS(C)(=O)=O)cc(C(N)=O)cc2c1Br ; CCOP(=O)(O)C(F)(F)c1sc2c(OCCCS(C)(=O)=O)cc(C(N)=O)cc2c1C#N ; CS(=O)(=O)CCCOc1cc(C(N)=O)cc2c(C#N)c(C(F)(F)P(=O)(O)O)sc12]

### Step A: Synthesis of ethyl hydrogen ((5-carbamoyl-3-cyano-7-(3-(methylsulfonyl)propoxy)benzo[b]thiophen-2-yl)difluoromethyl)phosphonate

A mixture of diethyl ((3-bromo-5-carbamoyl-7-(3-(methylsulfonyl)propoxy)benzo[b]thiophen-2-yl)difluoromethyl)phosphonate (50 mg, 0.086 mmol), tetrakis(triphenylphosphine) palladium (0) (200 mg, 0.173 mmol), and zinc cyanide (11 μl, 0.17 mmol) in DMF (2 mL) was heated to 100° C., and allowed to stir at this temperature for 16 hours. The reaction mixture was directly purified using reverse phase HPLC (eluting acetonitrile in water) to provide ethyl hydrogen ((5-carbamoyl-3-cyano-7-(3-(methylsulfonyl)propoxy)benzo[b]thiophen-2-yl)difluoromethyl)phosphonate. MS (ESI, m/z) 497 (M+H)⁺

### Step B: Synthesis of ((5-carbamoyl-3-cyano-7-(3-(methylsulfonyl)propoxy)benzo[b]thiophen-2-yl)difluoromethyl)phosphonic acid

To a mixture of ethyl hydrogen ((5-carbamoyl-3-cyano-7-(3-(methylsulfonyl)propoxy)benzo[b]thiophen-2-yl)difluoromethyl)phosphonate (25 mg, 0.050 mmol) in DMF (0.26 mL) was added bromotrimethdylsilane (0.13 mL, 1.0 mmol). The mixture was stirred heated to 60° C., and allowed to stir at this temperature for 1.5 hours. The reaction mixture was quenched with ethanol (1 mL), concentrated in vacuo, and the resulting residue was purified using reverse phase HPLC [eluting acetonitrile in (water with 0.01 M ammonium bicarbonate)]to provide ((5-carbamoyl-3-cyano-7-(3-(methylsulfonyl)propoxy)benzo[b]thiophen-2-yl)difluoromethyl)phosphonic acid (Compound 421). ¹H NMR (400 MHz, D₂O) δ 7.70 (s, 1H), 7.21 (s, 1H), 4.40-4.31 (m, 2H), 3.54 (t, J=7.8 Hz, 2H), 3.17 (s, 3H), 2.46-2.38 (m, 2H). MS (ESI, m/z): 469 (M+H)⁺

The following illustrative compounds were made using the methods described in the Example immediately above, and substituting the appropriate reactants and/or reagents.

| Com- | | Mass |
|---|---|---|
| pound | Structure | [M + H]⁺ |
| 422 | 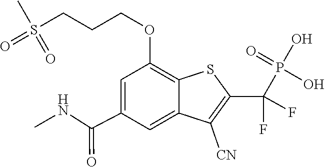 [SMILES: CNC(=O)c1cc(OCCCS(C)(=O)=O)c2sc(C(F)(F)P(=O)(O)O)c(C#N)c2c1] | 483 |
| 423 | 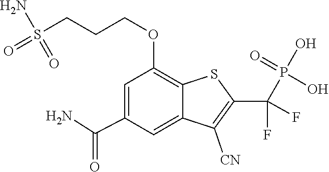 [SMILES: N#Cc1c(C(F)(F)P(=O)(O)O)sc2c(OCCCS(N)(=O)=O)cc(C(N)=O)cc12] | 468 [M − H]⁻ |
| 424 | 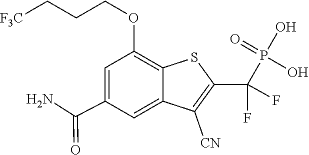 [SMILES: N#Cc1c(C(F)(F)P(=O)(O)O)sc2c(OCCCC(F)(F)F)cc(C(N)=O)cc12] | 459 |
| 425 | 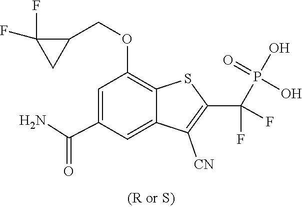 [SMILES: N#Cc1c(C(F)(F)P(=O)(O)O)sc2c(OCC3CC3(F)F)cc(C(N)=O)cc12] | 439 |
| 426 | 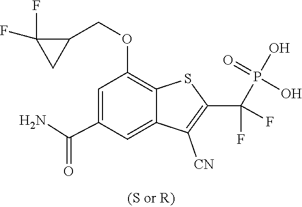 [SMILES: N#Cc1c(C(F)(F)P(=O)(O)O)sc2c(OCC3CC3(F)F)cc(C(N)=O)cc12] | 439 |
| 427 | 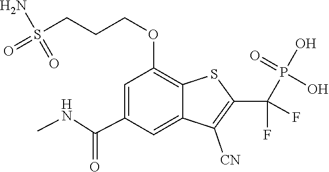 [SMILES: CNC(=O)c1cc(OCCCS(N)(=O)=O)c2sc(C(F)(F)P(=O)(O)O)c(C#N)c2c1] | 484 |
| 428 | 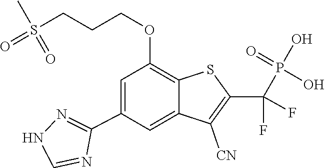 [SMILES: CS(=O)(=O)CCCOc1cc(-c2nc[nH]n2)cc2c(C#N)c(C(F)(F)P(=O)(O)O)sc12] | 491 [M − H]⁻ |

### Example 53

### Preparation of Compound 429

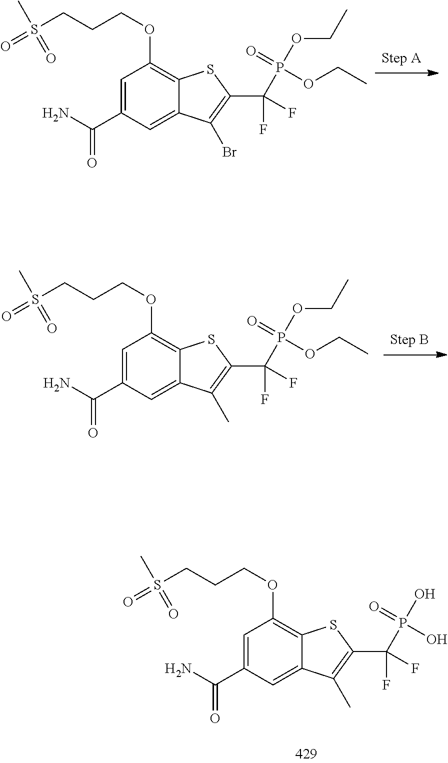 [SMILES: CCOP(=O)(OCC)C(F)(F)c1sc2c(OCCCS(C)(=O)=O)cc(C(N)=O)cc2c1Br ; CCOP(=O)(OCC)C(F)(F)c1sc2c(OCCCS(C)(=O)=O)cc(C(N)=O)cc2c1C ; Cc1c(C(F)(F)P(=O)(O)O)sc2c(OCCCS(C)(=O)=O)cc(C(N)=O)cc12]

### Step A: Synthesis of diethyl ((5-carbamoyl-3-methyl-7-(3-(methylsulfonyl)propoxy)benzo[b]thiophen-2-yl)difluoromethyl)phosphonate

A mixture of diethyl ((3-bromo-5-carbamoyl-7-(3-(methylsulfonyl)propoxy)benzo[b]thiophen-2-yl)difluoromethyl)phosphonate (50 mg, 0.086 mmol), bis(di-tert-butyl(4-dimethylaminophenyl)phosphine)dichloropalladium (II) (6 mg, 9 μmol), 2,4,6-trimethyl-1,3,5,2,4,6-trioxatriborinane (13 mg, 0.10 mmol), and potassium carbonate (60 mg, 0.43 mmol) in 1,4-dioxane (4 mL) was heated to 80° C., and allowed to stir at this temperature for 1 hour under an argon atmosphere. The reaction mixture was then diluted with water (20 mL), and the mixture was extracted with ethyl acetate (30 mL×3). The combined organic extracts were washed with brine (20 mL), dried over anhydrous sodium sulfate, and filtered. The filtrate was concentrated in vacuo, and the resulting residue was purified using reverse phase HPLC [eluting acetonitrile in (water with 0.01 M ammonium bicarbonate)]to provide diethyl ((5-carbamoyl-3-methyl-7-(3-(methylsulfonyl)propoxy)benzo[b]thiophen-2-yl)difluoromethyl)phosphonate. MS (ESI, m/z): 514 (M+H)⁺

### Step B: Synthesis of ((5-carbamoyl-3-methyl-7-(3-(methylsulfonyl)propoxy)benzo[b]thiophen-2-yl)difluoromethyl)phosphonic acid

To a mixture of diethyl ((5-carbamoyl-3-methyl-7-(3-(methylsulfonyl)propoxy)benzo[b]thiophen-2-yl)difluoromethyl)phosphonate (12 mg, 0.023 mmol) in DMF (182 μL) was added bromotrimethylsilane (91 μL, 0.69 mmol). The mixture was heated to 60° C., and allowed to stir at this temperature for 1.5 hours. The reaction was quenched with ethanol (0.2 mL), concentrated in vacuo, and the resulting residue was purified using reverse phase HPLC [eluting acetonitrile in (water with 0.01 M ammonium bicarbonate)]to provide ((5-carbamoyl-3-methyl-7-(3-(methylsulfonyl)propoxy) benzo[b]thiophen-2-yl)difluoromethyl)phosphonic acid (Compound 429). ¹H NMR (400 MHz, D₂O) δ 7.52 (s, 1H), 6.90 (s 1H), 4.16 (t, J=6.0 Hz, 2H), 3.52-3.44 (m, 2H), 3.15 (s, 3H), 2.37 (s, 3H), 2.35-2.29 (in, 2H). MS (ESI, m/z): 458 (M+H)⁺

The following illustrative compounds were made using the methods described in the Example immediately above, and substituting the appropriate reactants and/or reagents.

| Com- | | Mass |
|---|---|---|
| pound | Structure | [M + H]⁺ |
| 430 | 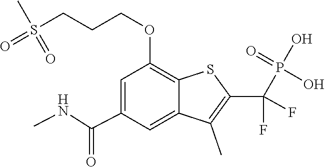 [SMILES: CNC(=O)c1cc(OCCCS(C)(=O)=O)c2sc(C(F)(F)P(=O)(O)O)c(C)c2c1] | 472 |
| 431 | 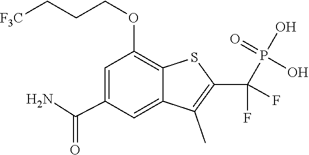 [SMILES: Cc1c(C(F)(F)P(=O)(O)O)sc2c(OCCCC(F)(F)F)cc(C(N)=O)cc12] | 448 |
| 432 | 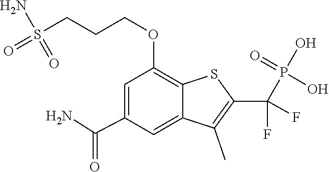 [SMILES: Cc1c(C(F)(F)P(=O)(O)O)sc2c(OCCCS(N)(=O)=O)cc(C(N)=O)cc12] | 459 |
| 433 | 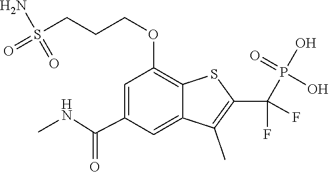 [SMILES: CNC(=O)c1cc(OCCCS(N)(=O)=O)c2sc(C(F)(F)P(=O)(O)O)c(C)c2c1] | 473 |
| 434^{a} | 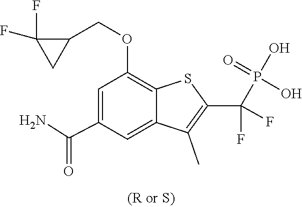 [SMILES: Cc1c(C(F)(F)P(=O)(O)O)sc2c(OCC3CC3(F)F)cc(C(N)=O)cc12] | 426 [M − H]⁻ |
| 435^{a} | 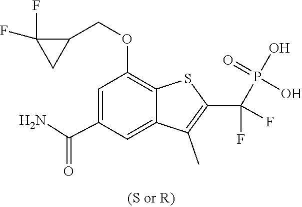 [SMILES: Cc1c(C(F)(F)P(=O)(O)O)sc2c(OCC3CC3(F)F)cc(C(N)=O)cc12] | 426 [M − H]⁻ |
| 436 | 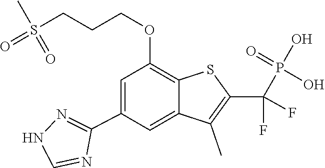 [SMILES: Cc1c(C(F)(F)P(=O)(O)O)sc2c(OCCCS(C)(=O)=O)cc(-c3nc[nH]n3)cc12] | 482 |
| 437 | 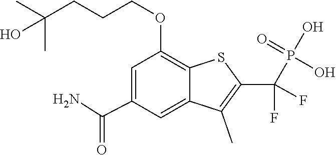 [SMILES: Cc1c(C(F)(F)P(=O)(O)O)sc2c(OCCCC(C)(C)O)cc(C(N)=O)cc12] | 436 [M − H]⁻ |
| 438 | 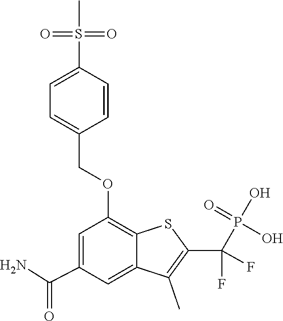 [SMILES: Cc1c(C(F)(F)P(=O)(O)O)sc2c(OCc3ccc(S(C)(=O)=O)cc3)cc(C(N)=O)cc12] | 506 |

^{a}Products were separated into pure stereoisomers prior to TMS-Br deprotection (after Step A) using the following condtions: ChiralArt Cellulose-SB; 4.6 x 100 mm, 3 um; 25% isopropanol in hexanes with 0.1% diethylamine; flow rate = 1 mL/min

### Example 54

### Preparation of Compound 439

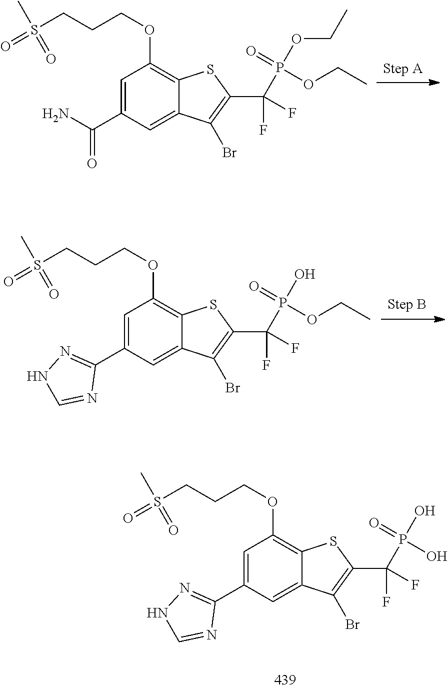 [SMILES: CCOP(=O)(OCC)C(F)(F)c1sc2c(OCCCS(C)(=O)=O)cc(C(N)=O)cc2c1Br ; CCOP(=O)(O)C(F)(F)c1sc2c(OCCCS(C)(=O)=O)cc(-c3nc[nH]n3)cc2c1Br ; CS(=O)(=O)CCCOc1cc(-c2nc[nH]n2)cc2c(Br)c(C(F)(F)P(=O)(O)O)sc12]

### Step A: Synthesis of ethyl hydrogen ((3-bromo-7-(3-(methylsulfonyl)propoxy)-5-(1H-1,2,4-triazol-3-yl)benzo[b]thiophen-2-yl)difluoromethyl)phosphonate

A mixture of diethyl ((3-bromo-5-carbamoyl-7-(3-(methylsulfonyl)propoxy)benzo[b]thiophen-2-yl)difluoromethyl)phosphonate (50 mg, 0.086 mmol) in N,N-dimethylformamide dimethyl acetal (0.50 mL) was heated to 80° C., and allowed to stir at this temperature for 2 hours. The reaction mixture was then concentrated in vacuo, and the resulting residue was taken up in acetic acid (0.5 mL). Hydrazine hydrate (98%, 43 mg, 0.86 mmol) was added, and the resulting reaction was heated to 90° C., and allowed to stir at this temperature for 2 hours. The reaction mixture was then directly purified using reverse phase HPLC (eluting acetonitrile in water) to provide ethyl hydrogen ((3-bromo-7-(3-(methylsulfonyl)propoxy)-5-(1H-1,2,4-triazol-3-yl)benzo[b]thiophen-2-yl)difluoromethyl)phosphonate. MS (ESI, m/z): 574, 576 (M+H)⁺

### Step B: ((3-bromo-7-(3-(methylsulfonyl)propoxy)-5-(1H-1,2,4-triazol-3-yl)benzo[b]thiophen-2-yl)difluoromethyl)phosphonic acid

To a mixture of ethyl hydrogen ((3-bromo-7-(3-(methylsulfonyl)propoxy)-5-(1H-1,2,4-triazol-3-yl)benzo[b]thiophen-2-yl)difluoromethyl)phosphonate (20 mg, 0.035 mmol) in DMF (182 μL) was added bromotrimethylsilane (91 μL, 1.1 mmol). The resulting reaction was heated to 60° C., and allowed to stir at this temperature for 2 hours. The reaction was quenched with ethanol (0.2 mL), concentrated in vacuo, and the resulting residue was purified using reverse phase HPLC [eluting acetonitrile in (water with 0.01 M ammonium bicarbonate)]to provide ((3-bromo-7-(3-(methylsulfonyl)propoxy)-5-(1H-1,2,4-triazol-3-yl)benzo[b]thiophen-2-yl)difluoromethyl)phosphonic acid (Compound 438). ¹H NMR (400 MHz, D₂O) δ 8.22 (s, 1H), 7.42 (s, 1H), 6.88 (s, 1H), 4.13 (t, J=6.0 Hz, 2H), 3.52-3.46 (m, 2H), 3.17 (s, 3H), 2.36-2.31 (m, 2H). MS (ESI, m/z): 546, 548 (M+H)⁺

The following illustrative compounds were made using the methods described in the Example immediately above, and substituting the appropriate reactants and/or reagents.

| Com- | | Mass |
|---|---|---|
| pound | Structure | [M + H]⁺ |
| 440 | 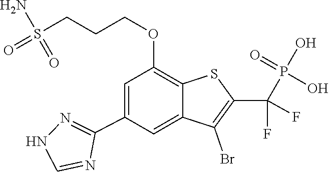 [SMILES: NS(=O)(=O)CCCOc1cc(-c2nc[nH]n2)cc2c(Br)c(C(F)(F)P(=O)(O)O)sc12] | 545, 547 [M − H]⁻ |
| 441 | 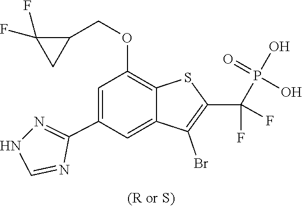 [SMILES: O=P(O)(O)C(F)(F)c1sc2c(OCC3CC3(F)F)cc(-c3nc[nH]n3)cc2c1Br] | 516, 518 |
| 442 | 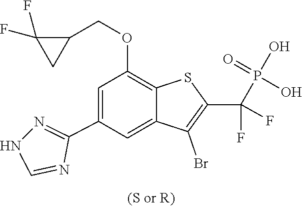 [SMILES: O=P(O)(O)C(F)(F)c1sc2c(OCC3CC3(F)F)cc(-c3nc[nH]n3)cc2c1Br] | 516, 518 |
| 443 | 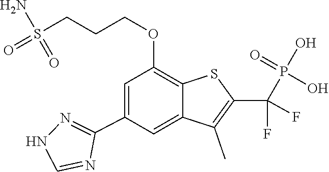 [SMILES: Cc1c(C(F)(F)P(=O)(O)O)sc2c(OCCCS(N)(=O)=O)cc(-c3nc[nH]n3)cc12] | 483 |
| 444 | 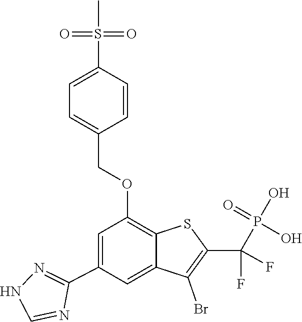 [SMILES: CS(=O)(=O)c1ccc(COc2cc(-c3nc[nH]n3)cc3c(Br)c(C(F)(F)P(=O)(O)O)sc23)cc1] | 594, 596 |

### Example 55

### Preparation of Compound 445

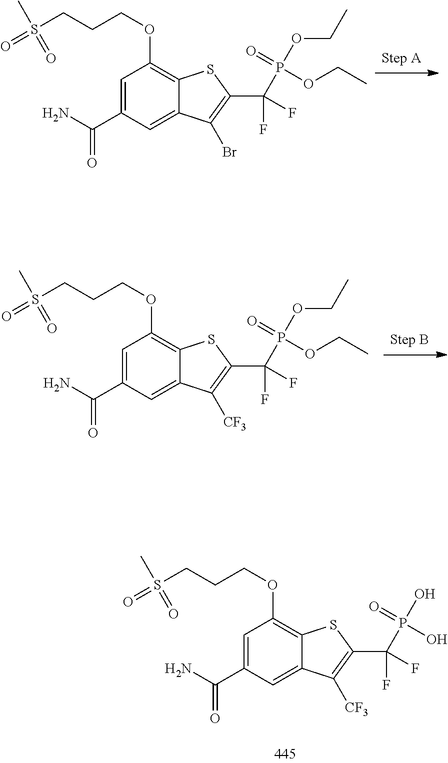 [SMILES: CCOP(=O)(OCC)C(F)(F)c1sc2c(OCCCS(C)(=O)=O)cc(C(N)=O)cc2c1Br ; CCOP(=O)(OCC)C(F)(F)c1sc2c(OCCCS(C)(=O)=O)cc(C(N)=O)cc2c1C(F)(F)F ; CS(=O)(=O)CCCOc1cc(C(N)=O)cc2c(C(F)(F)F)c(C(F)(F)P(=O)(O)O)sc12]

### Step A: Synthesis of diethyl ((5-carbamoyl-7-(3-(methylsulfonyl)propoxy)-3-(trifluoromethyl) benzo[b]thiophen-2-yl)difluoromethyl)phosphonate

To a stirred mixture of diethyl ((3-bromo-5-carbamoyl-7-(3-(methylsulfonyl)propoxy) benzo[b]thiophen-2-yl)difluoromethyl)phosphonate (50 mg, 0.086 mmol), and 4-methylmorpholine (0.50 mL) was added copper (I) iodide (16 mg, 0.086 mmol), and methyl 2,2-difluoro-2-(fluorosulfonyl)acetate (0.16 g, 0.86 mmol) at room temperature under argon atmosphere. The resulting reaction was heated to 80° C., and allowed to stir at this temperature for 4 hours. The reaction mixture was directly purified using reverse phase HPLC (eluting acetonitrile in water) to provide a crud product which was further purified using reverse phase HPLC (eluting acetonitrile in [water with 10 mM NH₄HCO₃]) to provide diethyl ((5-carbamoyl-7-(3-(methylsulfonyl)propoxy)-3-(trifluoromethyl)benzo[b]thiophen-2-yl)difluoromethyl)phosphonate. MS (ESI, m/z): 568 [M+H]⁺

### Step B: Synthesis of ((5-carbamoyl-7-(3-(methylsulfonyl)propoxy)-3-(trifluoromethyl)benzo[b]thiophen-2-yl)difluoromethyl)phosphonic acid

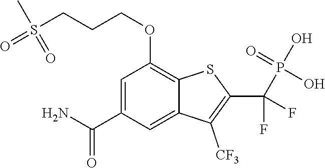 [SMILES: CS(=O)(=O)CCCOc1cc(C(N)=O)cc2c(C(F)(F)F)c(C(F)(F)P(=O)(O)O)sc12]

To a stirred mixture of diethyl ((5-carbamoyl-7-(3-(methylsulfonyl)propoxy)-3-(trifluoromethyl)benzo[b]thiophen-2-yl)difluoromethyl)phosphonate (8.0 mg, 0.014 mmol) in DMF (0.10 mL) was added bromotrimethylsilane (55 μL, 0.42 mmol). The resulting reaction was heated to 60° C., and allowed to stir at this temperature for 1.5 hours. The reaction was quenched with ethanol (0.1 mL), and concentrated in vacuo. The resulting residue was purified using reverse phase (eluting acetonitrile in [water with 10 mM NH₄HCO₃) to provide ((5-carbamoyl-7-(3-(methylsulfonyl)propoxy)-3-(trifluoromethyl)benzo[b]thiophen-2-yl)difluoromethyl)phosphonic acid (Compound 445). ¹H NMR (300 MHz, D₂O) δ 7.96 (s, 1H), 7.23 (s, 1H), 4.36 (t, J=5.7 Hz, 2H), 3.55 (t, J=7.7 Hz, 2H), 3.20 (s, 3H), 2.51-2.35 (m, 2H). MS (ESI, m/z): 512 [M+H]⁺

The following illustrative compounds were made using the methods described in the Example immediately above, and substituting the appropriate reactants and/or reagents.

| Com- | | Mass |
|---|---|---|
| pound | Structure | [M + H]⁺ |
| 446 | 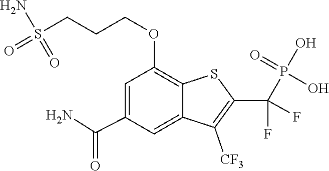 [SMILES: NC(=O)c1cc(OCCCS(N)(=O)=O)c2sc(C(F)(F)P(=O)(O)O)c(C(F)(F)F)c2c1] | 511 [M − H]⁻ |

### Example 56

### Preparation of Compound 447

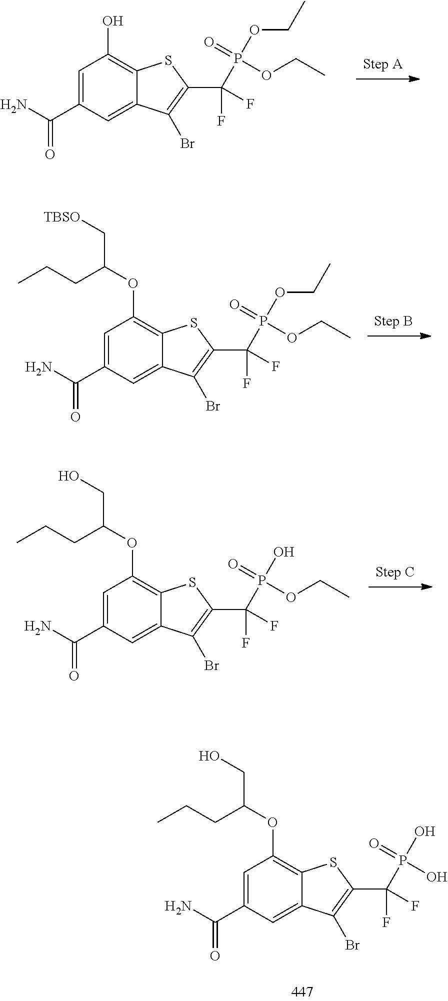

### Step A: Synthesis of diethyl ((3-bromo-7-((1-((tert-butyldimethylsilyl)oxy)pentan-2-yl)oxy)-5-carbamoylbenzo[b]thiophen-2-yl)difluoromethyl)phosphonate

To a flask containing diethyl ((3-bromo-5-carbamoyl-7-hydroxybenzo[b]thiophen-2-yl)difluoromethyl)phosphonate (0.22 g, 0.48 mmol) was added a mixture of 1-((tert-butyldimethylsilyl)oxy)pentan-2-ol (0.26 mg, 1.2 mmol) in toluene (3.2 mL). Cyanomethylenetributylphosphorane (0.13 mL, 0.48 mmol) was added and the resulting reaction was heated to 80° C., and allowed to stir at this temperature for 4 hours. The mixture was diluted with ethyl acetate and water, and the organic phase was collected, dried over magnesium sulfate, filtered and then concentrated in vacuo. The resulting residue was purified using silica gel chromatography (eluting ethyl acetate in petroleum ether) to provide diethyl ((3-bromo-7-((1-((tert-butyldimethylsilyl)oxy)pentan-2-yl)oxy)-5-carbamoylbenzo[b]thiophen-2-yl)difluoromethyl)phosphonate. MS (ESI, m/z): 658, 660 [M+H]⁺

### Step B: Synthesis of ethyl hydrogen ((3-bromo-5-carbamoyl-7-((1-hydroxypentan-2-yl)oxy)benzo[b]thiophen-2-yl)difluoromethyl)phosphonate

To a flask containing diethyl ((3-bromo-7-((1-((tert-butyldimethylsilyl)oxy)pentan-2-yl)oxy)-5-carbamoylbenzo[b]thiophen-2-yl)difluoromethyl)phosphonate (83 mg, 0.13 mmol) was added THF (1.2 ml), and then TBAF (1.0 M in THF, 0.38 mL, 0.38 mmol). The resulting reaction was heated to 50° C., and allowed to stir for 1 hour. The reaction mixture was diluted with ethyl acetate and water, and the organic phase was collected, dried over magnesium sulfate, filtered, and concentrated in vacuo. The resulting residue was purified using silica gel chromatography (eluting ethyl acetate in petroleum ether) to provide ethyl hydrogen ((3-bromo-5-carbamoyl-7-((1-hydroxypentan-2-yl)oxy)benzo[b]thiophen-2-yl)difluoromethyl)phosphonate. MS (ESI, m/z): 516, 518 [M+H]⁺

### Step C: Synthesis of ((3-bromo-5-carbamoyl-7-((1-hydroxypentan-2-yl)oxy)benzo[b]thiophen-2-yl)difluoromethyl)phosphonic acid

To a flask containing ethyl hydrogen ((3-bromo-5-carbamoyl-7-((1-hydroxypentan-2-yl)oxy)benzo[b]thiophen-2-yl)difluoromethyl)phosphonate (31 mg, 0.060 mmol) was added DMF (0.60 mL), and bromotrimethylsilane (0.39 mL, 3.0 mmol). The resulting reaction was heated to 60° C., and allowed to stir at this temperature for 3 hours. The reaction was quenched with methanol (1.0 mL), and then directly purified using reverse phase HPLC (eluting acetonitrile in water, with a 0.1% TFA modifier) to provide ((3-bromo-5-carbamoyl-7-((1-hydroxypentan-2-yl)oxy)benzo[b]thiophen-2-yl)difluoromethyl)phosphonic acid (Compound 447). ¹H NMR (500 MHz, DMSO-d₆) δ 8.26 (s, 1H), 8.00 (s, 1H), 7.64 (s, 1H), 7.53 (s, 1H), 4.73-4.67 (m, 1H), 3.65-3.61 (m, 2H), 3.04-2.97 (m, 1H), 1.77-1.63 (m, 2H), 1.50-1.34 (m, 2H), 0.93-0.88 (m, 3H). MS (ESI, m/z): 488, 490 [M+H]⁺

### Example 57

### Preparation of Compound 448

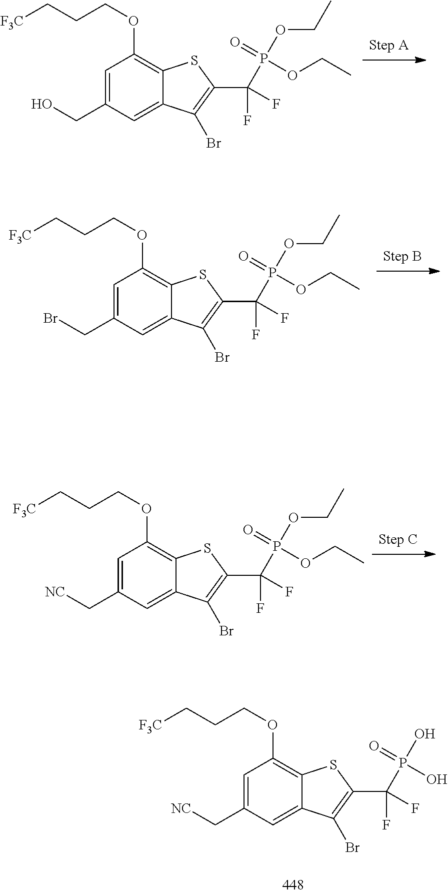 [SMILES: CCOP(=O)(OCC)C(F)(F)c1sc2c(OCCCC(F)(F)F)cc(CO)cc2c1Br ; CCOP(=O)(OCC)C(F)(F)c1sc2c(OCCCC(F)(F)F)cc(CBr)cc2c1Br ; CCOP(=O)(OCC)C(F)(F)c1sc2c(OCCCC(F)(F)F)cc(CC#N)cc2c1Br ; N#CCc1cc(OCCCC(F)(F)F)c2sc(C(F)(F)P(=O)(O)O)c(Br)c2c1]

### Step A: Synthesis of diethyl ((3-bromo-5-(bromomethyl)-7-(4,4,4-trifluorobutoxy)benzo[b]thiophen-2-yl)difluoromethyl)phosphonate

To a mixture of diethyl ((3-bromo-5-(hydroxymethyl)-7-(4,4,4-trifluorobutoxy)benzo [b]thiophen-2-yl)difluoromethyl)phosphonate (0.15 g, 0.27 mmol) in DCM (2.0 mL) was added tribromophosphane (0.15 g, 0.54 mmol) at 0° C. under an argon atmosphere. The resulting reaction was warmed to room temperature, and allowed to stir at this temperature for 2.5 hours. The reaction mixture was cooled to 0° C., and adjusted to pH 8 with saturated aqueous sodium bicarbonate (100 mL). The resulting solution was extracted with ethyl acetate (3×100 mL), and the combined organic extracts were washed with brine (200 mL), dried over anhydrous sodium sulfate, filtered, and concentrated in vacuo. The resulting residue was purified using silica gel chromatography (eluting ethyl acetate in petroleum ether) to provide diethyl ((3-bromo-5-(bromomethyl)-7-(4,4,4-trifluorobutoxy)benzo[b]thiophen-2-yl)difluoromethyl)phosphonate. MS (ESI, m/z): 617, 619 and 621 [M+H]⁺

### Step B: Synthesis of diethyl ((3-bromo-5-(cyanomethyl)-7-(4,4,4-trifluorobutoxy)benzo[b]thiophen-2-yl)difluoromethyl)phosphonate

To a mixture of diethyl ((3-bromo-5-(bromomethyl)-7-(4,4,4-trifluorobutoxy)benzo[b]thiophen-2-yl)difluoromethyl)phosphonate (0.040 g, 0.065 mmol) in acetonitrile (0.40 mL) was added trimethylsilanecarbonitrile (13 mg, 0.13 mmol) at room temperature. The resulting reaction was allowed to sit at room temperature for 1 hour, then the reaction mixture was cooled to 0° C., and tetrabutylammonium fluoride (1.0 M in THF, 0.13 mL, 0.13 mmol) was added. The resulting reaction was warmed to room temperature, and allowed to stir at this temperature for 2 hours. The reaction mixture was then filtered, and concentrated in vacuo. The resulting residue was purified using reverse phase HPLC (eluting acetonitrile in water) to provide diethyl ((3-bromo-5-(cyanomethyl)-7-(4,4,4-trifluorobutoxy)benzo[b]thiophen-2-yl)difluoromethyl)phosphonate. MS (ESI, m/z): 564, 566 [M+H]⁺

### Step C: Synthesis of ((3-bromo-5-(cyanomethyl)-7-(4,4,4-trifluorobutoxy)benzo[b]thiophen-2-yl)difluoromethyl)phosphonic acid

To a mixture of diethyl ((3-bromo-5-(cyanomethyl)-7-(4,4,4-trifluorobutoxy)benzo[b]thiophen-2-yl)difluoromethyl)phosphonate (0.020 g, 0.035 mmol) in DMF (0.28 mL) was added bromotrimethylsilane (0.14 mL, 1.1 mmol). The mixture was heated to 60° C., and allowed to stir at this temperature for 1.5 hours. The reaction was quenched with ethanol (1.0 mL), and concentrated in vacuo. The resulting residue was purified using reverse phase HPLC (eluting acetonitrile in [water with 10 mM NH₄HCO₃]) to provide ((3-bromo-5-(cyanomethyl)-7-(4,4,4-trifluorobutoxy)benzo[b]thiophen-2-yl)difluoromethyl)phosphonic acid (Compound 448). ¹H NMR: (300 MHz, D₂O) δ 7.29 (s, 1H), 6.78 (s, 1H), 4.18 (t, J=6.1 Hz, 2H), 3.93 (s, 2H), 2.44-2.27 (m, 2H), 2.08-1.99 (m, 2H). MS (ESI, m/z): 508, 510 [M+H]⁺.

### Example 58

### Preparation of Compound 449

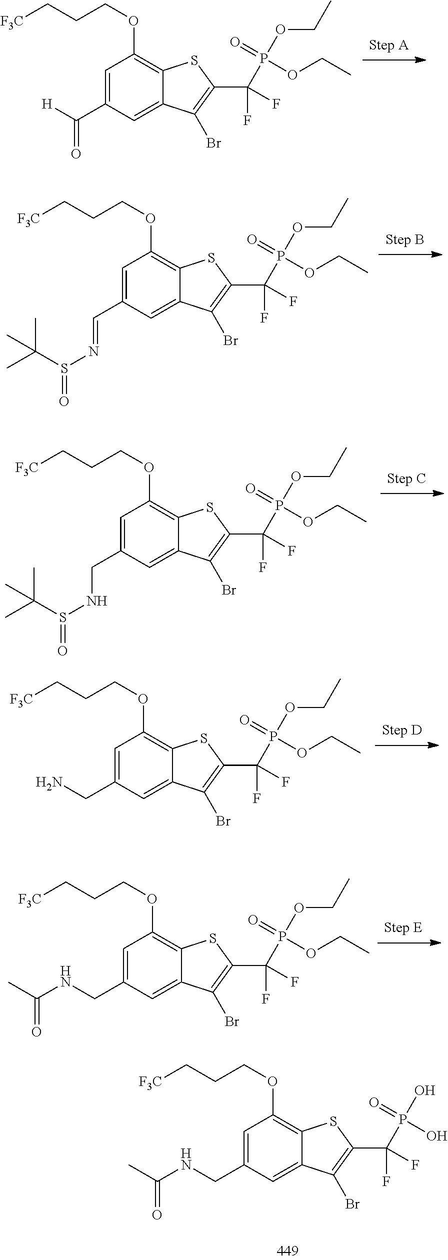

### Step A: Synthesis of diethyl ((3-bromo-5-(((tert-butylsulfinyl)imino)methyl)-7-(4,4,4-trifluorobutoxy)benzo[b]thiophen-2-yl)difluoromethyl)phosphonate

To a mixture of diethyl ((3-bromo-5-formyl-7-(4,4,4-trifluorobutoxy)benzo[b]thiophen-2-yl)difluoromethyl)phosphonate (0.10 g, 0.18 mmol) in THF (1.0 mL) was added 2-methylpropane-2-sulfinamide (44 mg, 0.36 mmol), and titanium (IV) ethoxide (0.11 mL, 0.54 mmol). The resulting reaction was heated to 75° C., and allowed to stir at this temperature for 2 hours. The reaction was quenched with water (100 mL), and then extracted with ethyl acetate (3×100 mL). The combined organic extracts were washed with brine (3×100 mL), dried over anhydrous sodium sulfate, filtered, and concentrated in vacuo. The resulting residue was purified using silica gel chromatography (eluting ethyl acetate in petroleum ether) to provide diethyl ((3-bromo-5-(((tert-butylsulfinyl)imino)methyl)-7-(4,4,4-trifluorobutoxy)benzo[b]thiophen-2-yl)difluoromethyl)phosphonate. MS (ESI, m/z): 656, 658 [M+H]⁺

### Step B: Synthesis of diethyl ((3-bromo-5-(((tert-butylsulfinyl)amino)methyl)-7-(4,4,4-trifluorobutoxy)benzo[b]thiophen-2-yl)difluoromethyl)phosphonate

To a mixture of diethyl ((3-bromo-5-(((tert-butylsulfinyl)imino)methyl)-7-(4,4,4-trifluorobutoxy)benzo[b]thiophen-2-yl)difluoromethyl)phosphonate (0.10 g, 0.15 mmol) in THF (1.0 mL) was added sodium borohydride (12 mg, 0.31 mmol). The resulting reaction was allowed to stir at room temperature for 2 hours, then the reaction was quenched with water (100 mL), and extracted with ethyl acetate (3×100 mL). The combined organic extracts were washed with brine (3×100 mL), dried over anhydrous sodium sulfate, filtered, and concentrated in vacuo. The resulting residue was purified using silica gel chromatography (eluting methanol in dichloromethane) to provide diethyl ((3-bromo-5-(((tert-butylsulfinyl)amino)methyl)-7-(4,4,4-trifluorobutoxy)benzo[b]thiophen-2-yl)difluoromethyl)phosphonate. MS (ESI, m/z): 658, 660 [M+H]⁺

### Step C: Synthesis of diethyl ((5-(aminomethyl)-3-bromo-7-(4,4,4-trifluorobutoxy)benzo[b]thiophen-2-yl)difluoromethyl)phosphonate

To a mixture of diethyl ((3-bromo-5-(((tert-butylsulfinyl)amino)methyl)-7-(4,4,4-trifluorobutoxy)benzo[b]thiophen-2-yl)difluoromethyl)phosphonate (0.080 g, 0.12 mmol) in methanol (1.0 mL) was added a solution of hydrogen chloride (4.0 M in 1,4-dioxane, 0.061 mL, 0.24 mmol). The resulting reaction was allowed to stir at room temperature for 1 hour. The reaction mixture was concentrated in vacuum and the resulting residue was diluted with water (30 mL), and then adjusted to pH 7 ˜8 with saturated aqueous sodium bicarbonate (2.0 mL). The resulting solution was extracted with ethyl acetate (3×100 mL), and the combined organic extracts were washed with brine (3×100 mL), dried over anhydrous sodium sulfate, filtered, and concentrated in vacuo. The resulting residue was purified using silica gel chromatography (eluting methanol in dichloromethane) to provide diethyl ((5-(aminomethyl)-3-bromo-7-(4,4,4-trifluorobutoxy)benzo[b]thiophen-2-yl)difluoromethyl)phosphonate. MS (ESI, m/z): 554, 556 [M+H]⁺

### Step D: Synthesis of diethyl ((5-(acetamidomethyl)-3-bromo-7-(4,4,4-trifluorobutoxy) benzo[b]thiophen-2-yl)difluoromethyl)phosphonate

To a mixture of diethyl ((5-(aminomethyl)-3-bromo-7-(4,4,4-trifluorobutoxy)benzo[b]thiophen-2-yl)difluoromethyl)phosphonate (25 mg, 0.045 mmol) in DCM (0.30 mL) was added triethylamine (13 μL, 0.090 mmol), N,N-dimethylpyridin-4-amine (0.55 mg, 4.5 μmol), and acetic anhydride (4.2 μL, 0.045 mmol) at 0° C. under an argon atmosphere. The resulting reaction was warmed to room temperature and allowed to stir at this temperature for 1 hour. The reaction mixture was directly purified using reverse phase HPLC (eluting acetonitrile in water) to provide diethyl ((5-(acetamidomethyl)-3-bromo-7-(4,4,4-trifluorobutoxy)benzo[b]thiophen-2-yl)difluoromethyl)phosphonate. MS (ESI, m/z): 596, 598 [M+H]⁺

### Step E: Synthesis of ((5-(acetamidomethyl)-3-bromo-7-(4,4,4-trifluorobutoxy)benzo[b]thiophen-2-yl)difluoromethyl)phosphonic acid

To a mixture of diethyl ((5-(acetamidomethyl)-3-bromo-7-(4,4,4-trifluorobutoxy)benzo[b]thiophen-2-yl)difluoromethyl)phosphonate (6.0 mg, 10 μmol) in DMF (80 μL) was added bromotrimethylsilane (39 μL, 0.30 mmol). The resulting reaction was heated to 60° C. and allowed to stir at this temperature for 1.5 hours. The reaction was quenched with ethanol (0.10 mL) and concentrated in vacuo. The resulting residue was purified using reverse phase HPLC (eluting acetonitrile in [water with 10 mM NH₄HCO₃]) to provide ((5-(acetamidomethyl)-3-bromo-7-(4,4,4-trifluorobutoxy)benzo[b]thiophen-2-yl)difluoromethyl)phosphonic acid (Compound 449). ¹H NMR: (300 MHz, D₂O) δ 7.46 (s, 1H), 6.95 (s, 1H), 4.50 (s, 2H), 4.33 (t, J=6.0 Hz, 2H), 2.54-2.32 (m, 2H), 2.19-2.09 (m, 2H), 2.07 (s, 3H). MS (ESI, m/z): 540, 542 [M+H]⁺

### Example 59

### Preparation of Compound 450

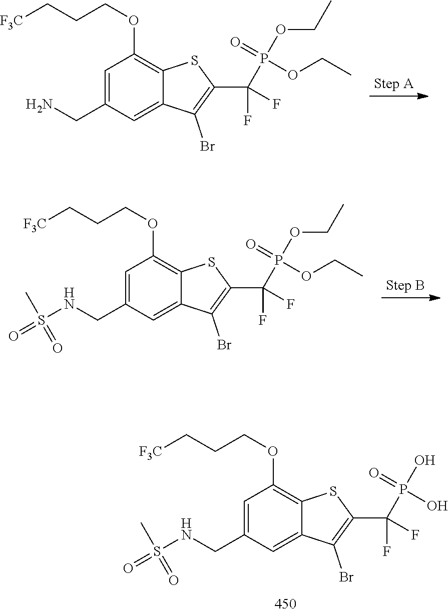 [SMILES: CCOP(=O)(OCC)C(F)(F)c1sc2c(OCCCC(F)(F)F)cc(CN)cc2c1Br ; CCOP(=O)(OCC)C(F)(F)c1sc2c(OCCCC(F)(F)F)cc(CNS(C)(=O)=O)cc2c1Br ; CS(=O)(=O)NCc1cc(OCCCC(F)(F)F)c2sc(C(F)(F)P(=O)(O)O)c(Br)c2c1]

### Step A: Synthesis of diethyl ((3-bromo-5-(methylsulfonamidomethyl)-7-(4,4,4-trifluorobutoxy) benzo[b]thiophen-2-yl)difluoromethyl)phosphonate

To a mixture of diethyl ((5-(aminomethyl)-3-bromo-7-(4,4,4-trifluorobutoxy)benzo[b]thiophen-2-yl)difluoromethyl)phosphonate (25 mg, 0.045 mmol) in DCM (0.30 mL) was added triethylamine (9.4 μL, 0.068 mmol), and methanesulfonyl chloride (3.5 μL, 0.045 mmol) at 0° C. under an argon atmosphere. The resulting reaction was warmed to room temperature, and allowed to stir at this temperature for 1 hour. The reaction mixture was concentrated in vacuo, and the resulting residue was purified using reverse phase HPLC (eluting acetonitrile in water) to provide diethyl ((3-bromo-5-(methylsulfonamidomethyl)-7-(4,4,4-trifluorobutoxy)benzo[b]thiophen-2-yl)difluoromethyl) phosphonate. MS (ESI, m/z): 632, 634 [M+H]⁺

### Step B: Synthesis of ((3-bromo-5-(methylsulfonamidomethyl)-7-(4,4,4-trifluorobutoxy)benzo[b]thiophen-2-yl)difluoromethyl)phosphonic acid

To a mixture of diethyl ((3-bromo-5-(methylsulfonamidomethyl)-7-(4,4,4-trifluorobutoxy) benzo[b]thiophen-2-yl)difluoromethyl)phosphonate (5.0 mg, 7.9 μmol) in DMF (60 μL) was added bromotrimethylsilane (31 μL, 0.24 mmol). The mixture was heated to 60° C., and allowed to stir for 1.5 hours. The reaction was quenched with ethanol (0.10 mL), and then concentrated in vacuo. The resulting residue was purified using reverse phase HPLC (eluting acetonitrile in [water with 10 mM NH₄HCO₃]) to provide ((3-bromo-5-(methylsulfonamidomethyl)-7-(4,4,4-trifluorobutoxy)benzo[b]thiophen-2-yl)difluoromethyl)phosphonic acid (Compound 450). ¹H NMR (400 MHz, D₂O) δ 7.50 (s, 1H), 7.01 (s, 1H), 4.40 (s, 2H), 4.32 (t, J=6.2 Hz, 2H), 2.95 (s, 3H), 2.51-2.37 (m, 2H), 2.17-2.09 (m, 2H). MS (ESI, m/z): 574, 576 [M−H]⁻

### Example 60

### Preparation of Compound 451

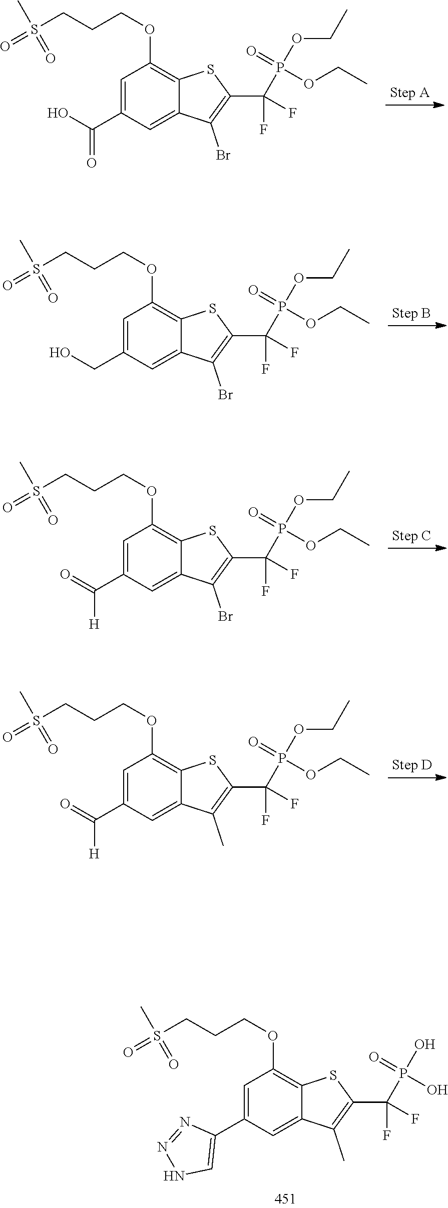 [SMILES: CCOP(=O)(OCC)C(F)(F)c1sc2c(OCCCS(C)(=O)=O)cc(C(=O)O)cc2c1Br ; CCOP(=O)(OCC)C(F)(F)c1sc2c(OCCCS(C)(=O)=O)cc(CO)cc2c1Br ; CCOP(=O)(OCC)C(F)(F)c1sc2c(OCCCS(C)(=O)=O)cc(C=O)cc2c1Br ; CCOP(=O)(OCC)C(F)(F)c1sc2c(OCCCS(C)(=O)=O)cc(C=O)cc2c1C ; Cc1c(C(F)(F)P(=O)(O)O)sc2c(OCCCS(C)(=O)=O)cc(-c3c[nH]nn3)cc12]

### Step A: Synthesis of diethyl ((3-bromo-5-(hydroxymethyl)-7-(3-(methylsulfonyl)propoxy)benzo[b]thiophen-2-yl)difluoromethyl)phosphonate

Borane-tetrahydrofuran complex (1.0 M in THF, 4.3 mL, 4.3 mmol) was added to a mixture of 3-bromo-2-((diethoxyphosphoryl)difluoromethyl)-7-(3-(methylsulfonyl)propoxy)benzo[b]thiophene-5-carboxylic acid (250 mg, 0.432 mmol) in THF (2.1 mL). The resulting reaction was allowed to stir at room temperature for 1 hour. The reaction was quenched with MeOH and water, and then extracted with DCM (3×50 mL). The combined organic extracts were dried over anhydrous sodium sulfate, filtered, and concentrated in vacuo to provide diethyl ((3-bromo-5-(hydroxymethyl)-7-(3-(methylsulfonyl)propoxy)benzo[b]thiophen-2-yl)difluoromethyl)phosphonate, which was used without purification in the next step. MS (ESI, m/z): 565, 567 (M+H)⁺

### Step B: Synthesis of diethyl ((3-bromo-5-formyl-7-(3-(methylsulfonyl)propoxy)benzo[b]thiophen-2-yl)difluoromethyl)phosphonate

Dess-Martin periodinane (225 mg, 0.531 mmol) was added to a mixture of diethyl ((3-bromo-5-(hydroxymethyl)-7-(3-(methylsulfonyl)propoxy)benzo[b]thiophen-2-yl)difluoromethyl)phosphonate (250 mg, 0.442 mmol) in DCM (2.2 mL). The resulting reaction was allowed to stir at room temperature for 15 minutes. The reaction was quenched with saturated aqueous Na₂S₂O₃ and extracted with DCM. The organic phase was collected, dried over anhydrous sodium sulfate, filtered, and concentrated in vacuo. The resulting residue was purified using silica gel chromatography (eluting EtOAc/EtOH (3:1) in petroleum ether) to provide diethyl ((3-bromo-5-formyl-7-(3-(methylsulfonyl)propoxy)benzo[b]thiophen-2-yl)difluoromethyl)phosphonate. MS (ESI, m/z): 563, 565 (M+H)⁺

### Step C: Synthesis of diethyl (difluoro(5-formyl-3-methyl-7-(3-(methylsulfonyl)propoxy)benzo[b]thiophen-2-yl)methyl)phosphonate

A mixture of diethyl ((3-bromo-5-formyl-7-(3-(methylsulfonyl)propoxy)benzo[b]thiophen-2-yl)difluoromethyl)phosphonate (25 mg, 0.044 mmol), trimethylboroxine (12 μl, 0.089 mmol), bis(di-tert-butyl(4-dimethylaminophenyl)phosphine)dichloropalladium (II) (6.3 mg, 8.9 μmol), and potassium carbonate (31 mg, 0.22 mmol) in 1,4-dioxane (0.44 mL) was heated to 120° C. in a microwave reactor for 6 minutes. The reaction mixture was diluted with water and extracted with DCM. The organic layer was collected, dried over anhydrous sodium sulfate, filtered, and concentrated in vacuo. The resulting residue was purified using silica gel chromatography (eluting EtOAc/EtOH (3:1) in petroleum ether) to provide diethyl (difluoro(5-formyl-3-methyl-7-(3-(methylsulfonyl)propoxy)benzo[b]thiophen-2-yl)methyl)phosphonate. MS (ESI, m/z): 521 (M+Na)⁺

### Step D: Synthesis of (difluoro(3-methyl-7-(3-(methylsulfonyl)propoxy)-5-(1H-1,2,3-triazol-4-yl)benzo[b]thiophen-2-yl)methyl)phosphonic acid

A mixture of diethyl (difluoro(5-formyl-3-methyl-7-(3-(methylsulfonyl)propoxy)benzo[b]thiophen-2-yl)methyl)phosphonate (25 mg, 0.050 mmol), nitromethane (8 μl, 0.2 mmol), ammonium acetate (4 mg, 0.05 mmol), sodium azide (8 mg, 0.1 mmol), and acetic acid (6 μl, 0.1 mmol) in DMF (0.3 mL) was heated to 130° C., and allowed to stir at this temperature for 15 minutes. The reaction mixture was cooled to room temperature and TMS-Br (130 μl, 1.0 mmol) was added. The resulting reaction was heated to 60° C., and allowed to stir at this temperature for 1 hour. The reaction was quenched with MeOH, and the mixture was directly purified using reverse phase HPLC (eluting acetonitrile in water, with 0.1% TFA modifier) to provide (difluoro(3-methyl-7-(3-(methylsulfonyl)propoxy)-5-(1H-1,2,3-triazol-4-yl)benzo[b]thiophen-2-yl)methyl)phosphonic acid (Compound 451). ¹H NMR (600 MHz, DMSO-d₆) δ 8.53 (s, 1H), 7.98 (s, 1H), 7.54 (s, 1H), 4.42 (t, J=6.2 Hz, 2H), 3.38-3.33 (m, 2H), 3.07 (s, 3H), 2.55 (s, 3H), 2.28 (dt, J=13.9, 6.2 Hz, 2H). MS (ESI, m/z): 482 (M+H)⁺

### Example 61

### Preparation of Compound 452

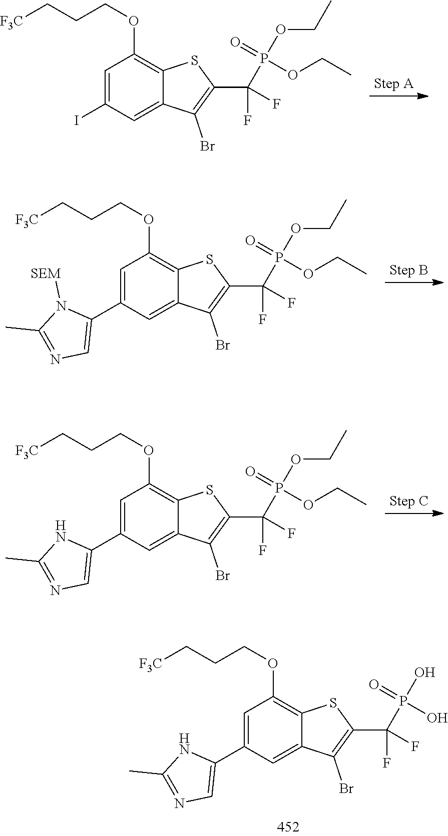

### Step A: Synthesis of diethyl ((3-bromo-5-(2-methyl-1-((2-(trimethylsilyl)ethoxy)methyl)-1H-imidazol-5-yl)-7-(4,4,4-trifluorobutoxy)benzo[b]thiophen-2-yl)difluoromethyl)phosphonate

To a mixture of diethyl ((3-bromo-5-iodo-7-(4,4,4-trifluorobutoxy)benzo[b]thiophen-2-yl)difluoromethyl)phosphonate (0.050 g, 0.077 mmol) in toluene (0.50 mL) was added 2-methyl-5-(tributylstannyl)-1-((2-(trimethylsilyl)ethoxy)methyl)-1H-imidazole (39 mg, 0.077 mmol), and dichlorobis(triphenylphosphine)palladium (II) (5.4 mg, 7.7 μmol) at room temperature under an argon atmosphere. The resulting reaction was heated to 100° C., and allowed to stir at this temperature for 2 hours. The reaction mixture was cooled to room temperature, and directly purified using reverse phase HPLC (eluting acetonitrile in water) to provide diethyl ((3-bromo-5-(2-methyl-1-((2-(trimethylsilyl)ethoxy)methyl)-1H-imidazol-5-yl)-7-(4,4,4-trifluorobutoxy)benzo[b]thiophen-2-yl)difluoromethyl)phosphonate. MS (ESI, m/z): 735, 737 (M+H)⁺

### Step B: Synthesis of diethyl ((3-bromo-5-(2-methyl-1H-imidazol-5-yl)-7-(4,4,4-trifluorobutoxy) benzo[b]thiophen-2-yl)difluoromethyl)phosphonate

To a mixture of diethyl ((3-bromo-5-(2-methyl-1-((2-(trimethylsilyl)ethoxy)methyl)-1H-imidazol-5-yl)-7-(4,4,4-trifluorobutoxy)benzo[b]thiophen-2-yl)difluoromethyl)phosphonate (35 mg, 0.048 mmol) in DCM (0.40 mL) was added trifluoroacetic acid (0.40 mL, 5.2 mmol) at room temperature under an argon atmosphere. The resulting reaction was allowed to stir at room temperature for 16 hours, and then the reaction mixture was concentrated in vacuo. The resulting residue was purified using reverse phase HPLC [eluting acetonitrile in (water with 10 mM NH₄HCO₃)]to provide diethyl ((3-bromo-5-(2-methyl-1H-imidazol-5-yl)-7-(4,4,4-trifluorobutoxy)benzo[b]thiophen-2-yl)difluoromethyl)phosphonate. MS (ESI, m/z): 605, 607 (M+H)⁺

### Step C: Synthesis of ((3-bromo-5-(2-methyl-1H-imidazol-5-yl)-7-(4,4,4-trifluorobutoxy)benzo[b]thiophen-2-yl)difluoromethyl)phosphonic acid

To a mixture of diethyl ((3-bromo-5-(2-methyl-1H-imidazol-5-yl)-7-(4,4,4-trifluorobutoxy)benzo[b]thiophen-2-yl)difluoromethyl)phosphonate (0.010 g, 0.017 mmol) in DMF (0.13 mL) was added bromotrimethylsilane (64 μL, 0.50 mmol) at room temperature under an argon atmosphere. The resulting reaction was heated to 60° C., and allowed to stir at this temperature for 1.5 hours. The reaction was quenched with ethanol (0.10 mL), and concentrated in vacuo. The resulting residue was purified using reverse phase HPLC [eluting acetonitrile in (water with 10 mM NH₄HCO₃)]to provide ((3-bromo-5-(2-methyl-1H-imidazol-5-yl)-7-(4,4,4-trifluorobutoxy)benzo[b]thiophen-2-yl)difluoromethyl)phosphonic acid (Compound 452). ¹H NMR (300 MHz, DMSO-d₆) δ 7.60 (s, 1H), 7.49 (s, 1H), 7.20 (s, 1H), 4.37-4.23 (m, 2H), 2.59-2.52 (m, 2H), 2.45 (s, 3H), 2.06-1.95 (m, 2H). MS (ESI, m/z): 549, 551 (M+H)⁺

### Example 62

### Preparation of Compound 453

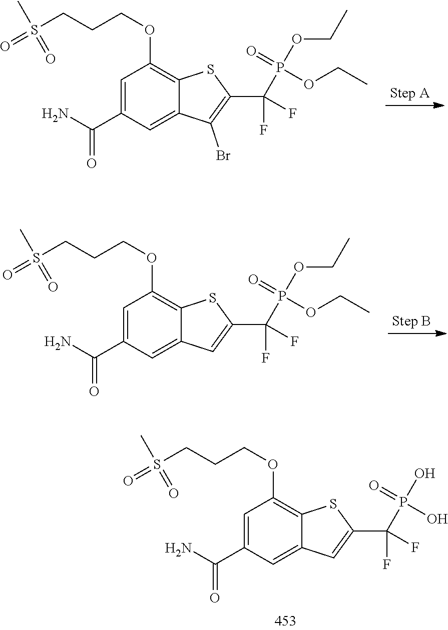 [SMILES: CCOP(=O)(OCC)C(F)(F)c1sc2c(OCCCS(C)(=O)=O)cc(C(N)=O)cc2c1Br ; CCOP(=O)(OCC)C(F)(F)c1cc2cc(C(N)=O)cc(OCCCS(C)(=O)=O)c2s1 ; CS(=O)(=O)CCCOc1cc(C(N)=O)cc2cc(C(F)(F)P(=O)(O)O)sc12]

### Step A: Synthesis of diethyl ((5-carbamoyl-7-(3-(methylsulfonyl)propoxy)benzo[b]thiophen-2-yl)difluoromethyl)phosphonate

To a mixture of diethyl ((3-bromo-5-carbamoyl-7-(3-(methylsulfonyl)propoxy)benzo[b]thiophen-2-yl)difluoromethyl)phosphonate (25 mg, 0.043 mmol) in dry THF (3 mL) was added Pd/C (10%, 25 mg) at room temperature under a hydrogen atmosphere (1 atm). The resulting reaction was allowed to stir for 2 hours at room temperature, then the reaction mixture was filtered, and concentrated in vacuo. The resulting residue was purified using reverse phase HPLC [eluting acetonitrile in (water with 10 mM NH₄HCO₃)]to provide diethyl ((5-carbamoyl-7-(3-(methylsulfonyl)propoxy)benzo[b]thiophen-2-yl)difluoromethyl)phosphonate MS (ESI, m/z): 500 (M+H)⁺

### Step B: Synthesis of ((5-carbamoyl-7-(3-(methylsulfonyl)propoxy)benzo[b]thiophen-2-yl)difluoromethyl)phosphonic acid

To diethyl ((5-carbamoyl-7-(3-(methylsulfonyl)propoxy)benzo[b]thiophen-2-yl)difluoromethyl)phosphonate (6.0 mg, 0.012 mmol) in DMF (96 μL) was added bromotrimethylsilane (48 μL, 0.36 mmol). The resulting reaction was heated to 60° C., and allowed to stir at this temperature for 1.5 hours. The reaction was quenched with ethanol (0.2 mL), concentrated in vacuo, and the resulting residue was purified using reverse phase HPLC [eluting acetonitrile in (water with 10 mM NH₄HCO₃)]to provide ammonium ((5-carbamoyl-7-(3-(methylsulfonyl)propoxy)benzo[b]thiophen-2-yl)difluoromethyl)phosphonate (Compound 453). ¹H NMR (400 MHz, D₂O) δ 7.81 (s, 1H), 7.68 (s, 1H), 7.11 (s, 1H), 4.29 (t, J=5.9 Hz, 2H), 3.53-3.46 (m, 2H), 3.14 (s, 3H), 2.40-2.31 (m, 2H). MS (ESI, m/z): 444 (M+H)⁺

### Example 63

### Preparation of Compound 454

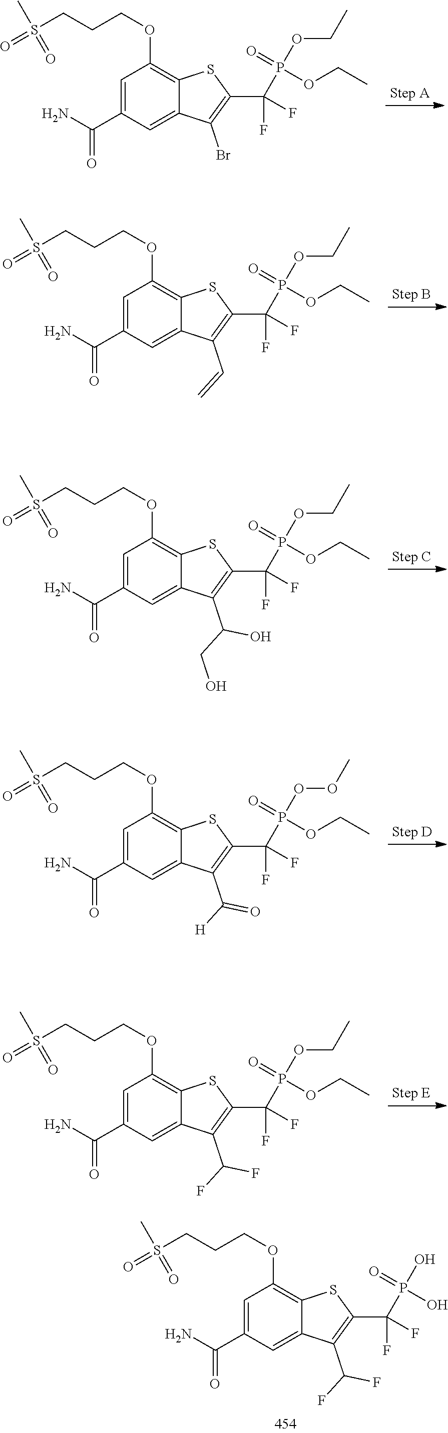

### Step A: Synthesis of diethyl ((5-carbamoyl-7-(3-(methylsulfonyl)propoxy)-3-vinylbenzo[b]thiophen-2-yl)difluoromethyl)phosphonate

To a mixture of diethyl ((3-bromo-5-carbamoyl-7-(3-(methylsulfonyl)propoxy)benzo[b]thiophen-2-yl)difluoromethyl)phosphonate (300 mg, 0.519 mmol) in 1,4-dioxane (5 mL) were added tetrakis(triphenylphosphine)palladium (0) (60 mg, 0.052 mmol), and tributyl(vinyl)tin (247 mg, 0.778 mmol) at room temperature. The resulting reaction was heated to 100° C., and allowed to stir at this temperature for 16 hours. The reaction mixture was concentrated in vacuo, and the resulting residue was purified using reverse phase HPLC (eluting acetonitrile in water) to provide diethyl ((5-carbamoyl-7-(3-(methylsulfonyl)propoxy)-3-vinylbenzo[b]thiophen-2-yl)difluoromethyl) phosphonate. MS (ESI, m/z): 526 (M+H)⁺

### Step B: Synthesis of diethyl ((5-carbamoyl-3-(1,2-dihydroxyethyl)-7-(3-(methylsulfonyl)propoxy)benzo[b]thiophen-2-yl)difluoromethyl)phosphonate

To a mixture of diethyl ((5-carbamoyl-7-(3-(methylsulfonyl)propoxy)-3-vinylbenzo[b]thiophen-2-yl)difluoromethyl)phosphonate (150 mg, 0.285 mmol) in acetone (2.7 mL), and water (0.3 mL) were added N-methylmorpholine-N-oxide (50 mg, 0.43 mmol), and a solution of osmium tetroxide (5% in water, 3.63 mg, 0.014 mmol) at 0° C. The resulting reaction was allowed to stir at 0° C. for 2 hours. The reaction was quenched with saturated aqueous Na₂S₂O₃ (80 mL), and extracted with ethyl acetate (80 mL×3). The combined organic extracts were washed with brine (80 mL×3), dried over anhydrous sodium sulfate, filtered, and concentrated in vacuo to provide diethyl ((5-carbamoyl-3-(1,2-dihydroxyethyl)-7-(3-(methylsulfonyl)propoxy)benzo[b]thiophen-2-yl)difluoromethyl)phosphonate, which was used directly in the next step without purification. MS (ESI, m/z): 560 (M+H)⁺

### Step C: Synthesis of diethyl ((5-carbamoyl-3-formyl-7-(3-(methylsulfonyl)propoxy)benzo[b]thiophen-2-yl)difluoromethyl)phosphonate

To a mixture of diethyl ((5-carbamoyl-3-(1,2-dihydroxyethyl)-7-(3-(methylsulfonyl)propoxy)benzo[b]thiophen-2-yl)difluoromethyl)phosphonate (160 mg, 0.286 mmol) in methanol (3 mL) was added a solution of sodium periodate (7% w/w in water, 0.961 mL, 0.315 mmol) in water at room temperature. The resulting reaction was allowed to stir at room temperature for 2 hours. The reaction was quenched with saturated aqueous Na₂S₂O₃ (20 mL), and extracted with ethyl acetate (50 mL×3). The combined organic extracts were washed with brine (50 mL), dried over anhydrous sodium sulfate, filtered, and concentrated in vacuo. The resulting residue was purified using silica gel chromatography (eluting methanol in dichloromethane) to provide diethyl ((5-carbamoyl-3-formyl-7-(3-(methylsulfonyl)propoxy)benzo[b]thiophen-2-yl)difluoromethyl)phosphonate. LCMS (ESI, m/z): 528 (M+H)⁺

### Step D: Synthesis of diethyl ((5-carbamoyl-3-(difluoromethyl)-7-(3-(methylsulfonyl)propoxy) benzo[b]thiophen-2-yl)difluoromethyl)phosphonate

To a mixture of diethyl ((5-carbamoyl-3-formyl-7-(3-(methylsulfonyl)propoxy)benzo[b]thiophen-2-yl)difluoromethyl)phosphonate (80 mg, 0.15 mmol) in dichloromethane (2 mL) was added diethylaminosulfur trifluoride (0.200 mL, 1.52 mmol) at 0° C. The resulting reaction was allowed to stir at room temperature for 16 hours. The reaction was quenched with saturated aqueous sodium bicarbonate (30 mL), and extracted with ethyl acetate (30 mL×3). The combined organic extracts were washed with brine (30 mL), dried over anhydrous sodium sulfate, filtered, and concentrated in vacuo. The resulting residue was purified using reverse phase HPLC (eluting acetonitrile in water) to provide diethyl ((5-carbamoyl-3-(difluoromethyl)-7-(3-(methylsulfonyl)propoxy)benzo[b]thiophen-2-yl)difluoromethyl)phosphonate. MS (ESI, m/z): 550 (M+H)⁺

### Step E: Synthesis of ((5-carbamoyl-3-(difluoromethyl)-7-(3-(methylsulfonyl)propoxy)benzo[b]thiophen-2-yl)difluoromethyl)phosphonic acid

To a mixture of diethyl ((5-carbamoyl-3-(difluoromethyl)-7-(3-(methylsulfonyl)propoxy) benzo[b]thiophen-2-yl)difluoromethyl)phosphonate (23 mg, 0.042 mmol) in N,N-dimethylformamide (0.2 mL) was added bromotrimethylsilane (0.109 mL, 0.837 mmol) at room temperature. The resulting reaction was heated to 50° C. for 2 hours, then reaction was quenched with ethanol (0.5 mL), and concentrated in vacuo. The resulting residue was purified using reverse phase HPLC [eluting acetonitrile in (water with 10 mM NH₄HCO₃)]to provide ((5-carbamoyl-3-(difluoromethyl)-7-(3-(methylsulfonyl)propoxy)benzo[b]thiophen-2-yl)difluoromethyl)phosphonic acid (Compound 454). ¹H NMR (400 MHz, D₂O) δ 8.03 (s, 1H), 7.38-7.06 (m, 2H), 4.26 (s, 2H), 3.44 (t, J=7.8 Hz, 2H), 3.08 (s, 3H), 2.33 (s, 2H). MS (ESI, m/z): 494 (M+H)⁺

### Example 64

### Preparation of Compound 455

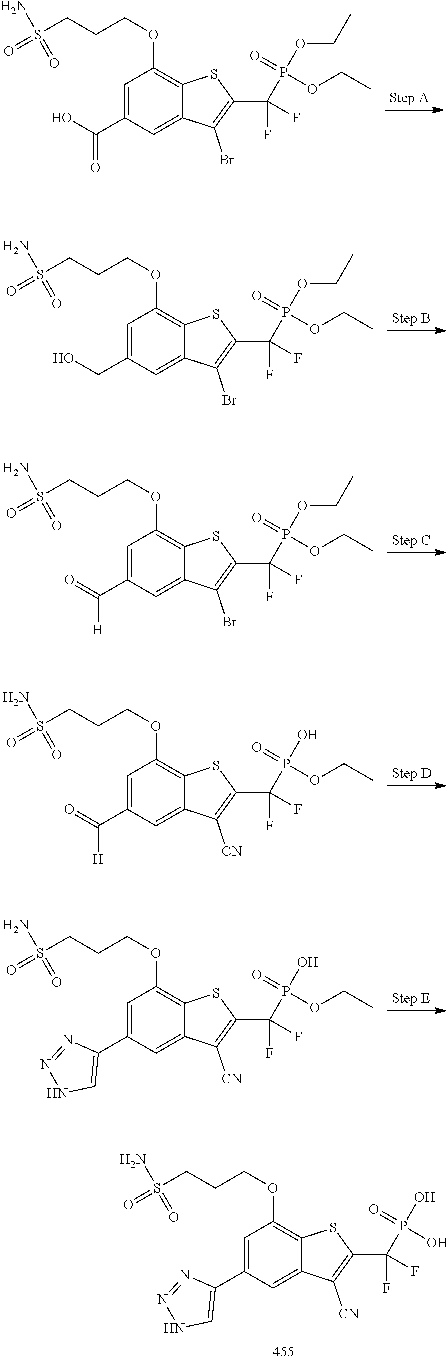 [SMILES: CCOP(=O)(OCC)C(F)(F)c1sc2c(OCCCS(N)(=O)=O)cc(C(=O)O)cc2c1Br ; CCOP(=O)(OCC)C(F)(F)c1sc2c(OCCCS(N)(=O)=O)cc(CO)cc2c1Br ; CCOP(=O)(OCC)C(F)(F)c1sc2c(OCCCS(N)(=O)=O)cc(C=O)cc2c1Br ; CCOP(=O)(O)C(F)(F)c1sc2c(OCCCS(N)(=O)=O)cc(C=O)cc2c1C#N ; CCOP(=O)(O)C(F)(F)c1sc2c(OCCCS(N)(=O)=O)cc(-c3c[nH]nn3)cc2c1C#N ; N#Cc1c(C(F)(F)P(=O)(O)O)sc2c(OCCCS(N)(=O)=O)cc(-c3c[nH]nn3)cc12]

### Step A: Synthesis of diethyl ((3-bromo-5-(hydroxymethyl)-7-(3-sulfamoylpropoxy)benzo[b]thiophen-2-yl)difluoromethyl)phosphonate

To a stirred solution of 3-bromo-2-((diethoxyphosphoryl)difluoromethyl)-7-(3-sulfamoylpropoxy)benzo[b]thiophene-5-carboxylic acid (140 mg, 0.241 mmol) in THF (1.5 mL) was added dropwise a solution of borane (1.0 M in THF, 2.17 mL, 2.2 mmol) at 0° C. under an argon atmosphere. The resulting reaction was allowed to stir at room temperature for 2 hours, then the reaction was quenched with methanol (10 mL), and water (30 mL). The mixture was extracted with ethyl acetate (50 mL×3), and the combined organic extracts were washed with brine (50 mL), dried over anhydrous sodium sulfate, filtered, and concentrated in vacuo. The resulting residue was purified using silica gel chromatography (eluting ethyl acetate in petroleum ether) to provide diethyl ((3-bromo-5-(hydroxymethyl)-7-(3-sulfamoylpropoxy)benzo[b]thiophen-2-yl)difluoromethyl)phosphonate. MS (ESI, m/z): 566, 568 (M+H)⁺

### Step B: Synthesis of diethyl ((3-bromo-5-formyl-7-(3-sulfamoylpropoxy)benzo[b]thiophen-2-yl)difluoromethyl)phosphonate

To a mixture of diethyl ((3-bromo-5-(hydroxymethyl)-7-(3-sulfamoylpropoxy)benzo[b]thiophen-2-yl)difluoromethyl)phosphonate (78 mg, 0.14 mmol) in DCM (0.8 mL) was added (1,1,1-triacetoxy)-1,1-dihydro-1,2-benziodoxol-3(1H)-one (117 mg, 0.275 mmol) at 0° C. under an argon atmosphere. The resulting reaction was allowed to stir at room temperature for 1 hour. The reaction was quenched with saturated aqueous sodium bicarbonate (30 mL), and extracted with ethyl acetate (100 mL×3). The combined organic extracts were washed with brine (100 mL×2), dried over anhydrous sodium sulfate, filtered, and concentrated in vacuo. The resulting residue was purified using silica gel chromatography (eluting ethyl acetate in petroleum ether) to provide diethyl ((3-bromo-5-formyl-7-(3-sulfamoylpropoxy)benzo[b]thiophen-2-yl)difluoromethyl)phosphonate. MS (ESI, m/z): 564, 566 (M+H)⁺

### Step C: Synthesis of ethyl hydrogen ((3-cyano-5-formyl-7-(3-sulfamoylpropoxy)benzo[b]thiophen-2-yl)difluoromethyl)phosphonate

To a mixture of diethyl ((3-bromo-5-formyl-7-(3-sulfamoylpropoxy)benzo[b]thiophen-2-yl)difluoromethyl)phosphonate (100 mg, 0.177 mmol) in N,N-dimethylformamide (2 mL) were added tetrakis(triphenylphosphine)palladium (0) (410 mg, 0.354 mmol), and zinc cyanide (41 mg, 0.35 mmol) at room temperature under an argon atmosphere. The resulting reaction was heated to 100° C., and allowed to stir at this temperature for 1.5 hours. The reaction mixture was cooled to room temperature, and directly purified using reverse phase HPLC (eluting acetonitrile in water) to provide ethyl hydrogen ((3-cyano-5-formyl-7-(3-sulfamoylpropoxy)benzo[b]thiophen-2-yl)difluoromethyl)phosphonate. MS (ESI, m/z): 483 (M+H)⁺

### Step D: Synthesis of ethyl hydrogen ((3-cyano-7-(3-sulfamoylpropoxy)-5-(1H-1,2,3-triazol-4-yl)benzo[b]thiophen-2-yl)difluoromethyl)phosphonate

To a stirred solution of ethyl hydrogen ((3-cyano-5-formyl-7-(3-sulfamoylpropoxy)benzo[b]thiophen-2-yl)difluoromethyl)phosphonate (20 mg, 0.041 mmol) in N,N-dimethylformamide (0.2 mL) were added acetic acid (20 mg, 0.33 mmol), nitromethane (5 mg, 0.08 mmol), sodium azide (5 mg, 0.08 mmol), and ammonium acetate (3 mg, 0.04 mmol) at room temperature under an argon atmosphere. The resulting reaction was heated to 140° C., and allowed to stir at this temperature for 1.5 hours. The reaction mixture was cooled to room temperature, and directly purified using reverse phase HPLC [eluting acetonitrile in (water with 10 mM NH₄HCO₃)]to provide ethyl hydrogen ((3-cyano-7-(3-sulfamoylpropoxy)-5-(1H-1,2,3-triazol-4-yl)benzo[b]thiophen-2-yl)difluoromethyl)phosphonate. MS (ESI, m/z): 522 (M+H)⁺

### Step E: Synthesis of ((3-cyano-7-(3-sulfamoylpropoxy)-5-(1H-1,2,3-triazol-4-yl)benzo[b]thiophen-2-yl)difluoromethyl)phosphonic acid

To a stirred solution of ethyl hydrogen ((3-cyano-7-(3-sulfamoylpropoxy)-5-(1H-1,2,3-triazol-4-yl)benzo[b]thiophen-2-yl)difluoromethyl)phosphonate (5 mg, 9 μmol) in N,N-dimethylformamide (100 μL) was added bromotrimethylsilane (36 μL, 0.28 mmol). The resulting reaction was heated to 60° C., and allowed to stir at this temperature for 3 hours. The reaction was quenched with ethanol (0.1 mL), and concentrated in vacuo. The resulting residue was purified using reverse phase HPLC [eluting acetonitrile in (water with 10 mM NH₄HCO₃)]to provide ((3-cyano-7-(3-sulfamoylpropoxy)-5-(1H-1,2,3-triazol-4-yl)benzo[b]thiophen-2-yl)difluoromethyl)phosphonic acid (Compound 455). ¹H NMR (400 MHz, DMSO-d₆) δ 8.50 (s, 1H), 7.94 (s, 1H), 7.56 (s, 1H), 4.52-4.28 (m, 2H), 3.31-3.12 (m, 2H), 2.31-2.13 (m, 2H). MS (ESI, m/z): 494 (M+H)⁺

### Example 65

### Preparation of Compound 456

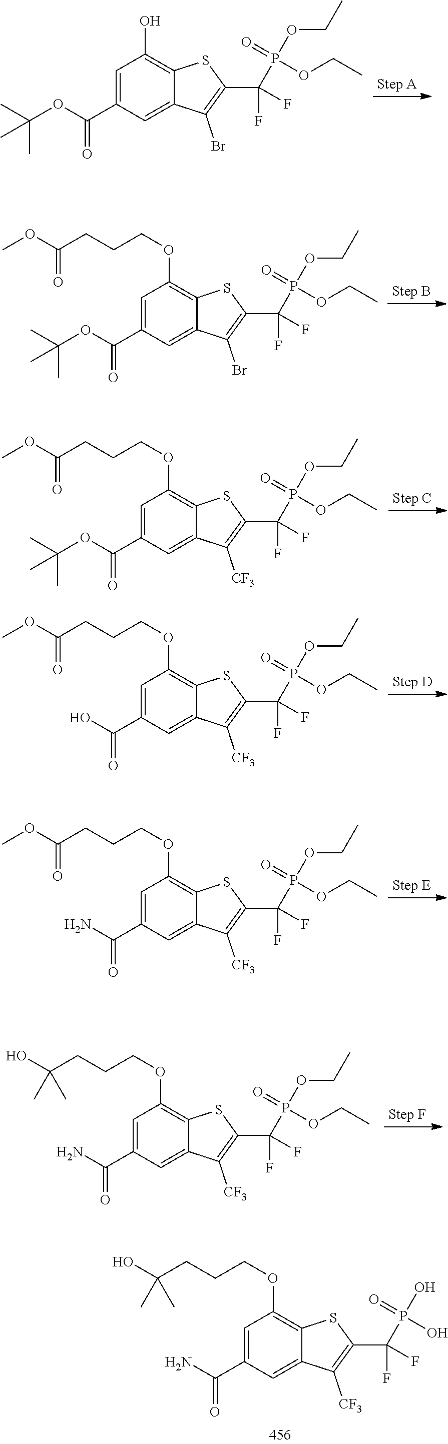

### Step A: Synthesis of tert-butyl 3-bromo-2-((diethoxyphosphoryl)difluoromethyl)-7-(4-methoxy-4-oxobutoxy)benzo[b]thiophene-5-carboxylate

To a solution of tert-butyl 3-bromo-2-((diethoxyphosphoryl)difluoromethyl)-7-hydroxybenzo[b]thiophene-5-carboxylate (500 mg, 0.970 mmol) in acetonitrile (5 mL) was added potassium carbonate (201 mg, 1.46 mmol), and methyl 4-bromobutanoate (527 mg, 2.91 mmol). The resulting reaction was heated to 50° C., and allowed to stir at this temperature for 16 hours. The reaction mixture was then diluted with DCM (80 mL), filtered through Celite, and washed with DCM. The filtrate was concentrated in vacuo, and the resulting residue was purified using silica gel chromatography (eluting ethyl acetate in petroleum ether) to provide tert-butyl 3-bromo-2-((diethoxyphosphoryl)difluoromethyl)-7-(4-methoxy-4-oxobutoxy)benzo[b]thiophene-5-carboxylate. MS (ESI, m/z): 615, 617 (M+H)⁺

### Step B: Synthesis of tert-butyl 2-((diethoxyphosphoryl)difluoromethyl)-7-(4-methoxy-4-oxobutoxy)-3-(trifluoromethyl)benzo[b]thiophene-5-carboxylate

To a mixture of tert-butyl 3-bromo-2-((diethoxyphosphoryl)difluoromethyl)-7-(4-methoxy-4-oxobutoxy)benzo[b]thiophene-5-carboxylate (600 mg, 0.975 mmol) in NMP (10 mL) were added methyl 2,2-difluoro-2-(fluorosulfonyl)acetate (1.87 g, 9.75 mmol), and copper (I) iodide (186 mg, 0.975 mmol) under an argon atmosphere. The resulting reaction was heated to 80° C., and was allowed to stir at this temperature for 2 hours. The reaction mixture was then directly purified using reverse phase HPLC (eluting acetonitrile in water, with 0.1% TFA) to provide tert-butyl 2-((diethoxyphosphoryl)difluoromethyl)-7-(4-methoxy-4-oxobutoxy)-3-(trifluoromethyl)benzo[b]thiophene-5-carboxylate. MS (ESI, m/z): 622 (M+H₂O)+

### Step C: Synthesis of 2-((diethoxyphosphoryl)difluoromethyl)-7-(4-methoxy-4-oxobutoxy)-3-(trifluoromethyl)benzo[b]thiophene-5-carboxylic acid

To a mixture of tert-butyl 2-((diethoxyphosphoryl)difluoromethyl)-7-(4-methoxy-4-oxobutoxy)-3-(trifluoromethyl)benzo[b]thiophene-5-carboxylate (360 mg, 0.596 mmol) in DCM (5 mL) was added 2,2,2-trifluoroacetic acid (5.0 mL, 65 mmol) at −20° C. under an argon atmosphere. The resulting reaction was heated to 40° C., and allowed to stir at this temperature for 1 hour. The reaction mixture was diluted with toluene (50 mL), and concentrated in vacuo. The resulting residue was taken up in MeCN (20 mL), and toluene (50 mL), and concentrated in vacuo to provide 2-((diethoxyphosphoryl)difluoromethyl)-7-(4-methoxy-4-oxobutoxy)-3-(trifluoromethyl)benzo[b]thiophene-5-carboxylic acid, which was used without purification in the next step. MS (ESI, m/z): 547 (M−H)⁻

### Step D: Synthesis of methyl 4-((5-carbamoyl-2-((diethoxyphosphoryl)difluoromethyl)-3-(trifluoromethyl)benzo[b]thiophen-7-yl)oxy)butanoate

To a stirred solution of 2-((diethoxyphosphoryl)difluoromethyl)-7-(4-methoxy-4-oxobutoxy)-3-(trifluoromethyl)benzo[b]thiophene-5-carboxylic acid (325 mg, 0.593 mmol, crude) in THF (5 mL) was added di(1H-imidazol-1-yl)methanone (250 mg, 1.54 mmol). The resulting reaction was allowed to stir for 16 hours at room temperature. A solution of ammonia (0.40 M in 1,4-dioxane, 14.8 mL, 5.9 mmol) was added to the reaction, and the mixture was allowed to stir for 2 hours at room temperature. The reaction mixture was concentrated in vacuo, and the resulting residue was taken up in ethyl acetate (50 mL), diluted with water (80 mL), and extracted with ethyl acetate (50 mL×2). The combined organic extracts were washed with brine (100 mL), dried over anhydrous sodium sulfate, filtered, and concentrated in vacuo. The resulting residue was purified using silica gel chromatography (eluting methanol in dichloromethane) to provide methyl 4-((5-carbamoyl-2-((diethoxyphosphoryl)difluoromethyl)-3-(trifluoromethyl)benzo[b]thiophen-7-yl)oxy)butanoate. MS (ESI, m/z): 548 (M+H)⁺

### Step E: Synthesis of diethyl ((5-carbamoyl-7-((4-hydroxy-4-methylpentyl)oxy)-3-(trifluoromethyl)benzo[b]thiophen-2-yl)difluoromethyl)phosphonate

To a mixture of methyl 4-((5-carbamoyl-2-((diethoxyphosphoryl)difluoromethyl)-3-(trifluoromethyl)benzo[b]thiophen-7-yl)oxy)butanoate (170 mg, 0.311 mmol) in ethyl ether (5 mL) was added a solution of methylmagnesium bromide (3.0 M in ethyl ether, 1.04 mL, 3.1 mmol) at −20° C. under an argon atmosphere. The resulting reaction was allowed to stir for 48 hours at −20° C., then the reaction was quenched with saturated aqueous ammonium chloride (50 mL) at −20° C. and extracted with ethyl acetate (50 mL×2). The combined organic extracts were washed with brine (100 mL), dried over anhydrous sodium sulfate, filtered, and concentrated in vacuo. The resulting residue was purified using silica gel chromatography (eluting methanol in dichloromethane) to provide diethyl ((5-carbamoyl-7-((4-hydroxy-4-methylpentyl)oxy)-3-(trifluoromethyl)benzo[b]thiophen-2-yl)difluoromethyl)phosphonate. MS (ESI, m/z): 546 (M−H)⁻

### Step F: Synthesis of ((5-carbamoyl-7-((4-hydroxy-4-methylpentyl)oxy)-3-(trifluoromethyl)benzo[b]thiophen-2-yl)difluoromethyl)phosphonic acid

To a mixture of diethyl ((5-carbamoyl-7-((4-hydroxy-4-methylpentyl)oxy)-3-(trifluoromethyl)benzo[b]thiophen-2-yl)difluoromethyl)phosphonate (50 mg, 0.091 mmol) in DMF (500 μL) was added bromotrimethylsilane (237 μL, 1.83 mmol). The resulting reaction was heated to 50° C., and allowed to stir at this temperature for 1 hour. The reaction was quenched with ethanol (1 mL), and directly purified using reverse phase HPLC [eluting acetonitrile in (water with 10 mM NH₄HCO₃)]to provide ((5-carbamoyl-7-((4-hydroxy-4-methylpentyl)oxy)-3-(trifluoromethyl)benzo [b]thiophen-2-yl)difluoromethyl)phosphonic acid (Compound 456). ¹H NMR (400 MHz, D₂O) δ 7.98 (s, 1H), 7.25 (s, 1H), 4.25 (t, J=6.4 Hz, 2H), 1.98-1.88 (m, 2H), 1.79-1.65 (m, 2H), 1.28 (s, 6H). MS (ESI, m/z): 490 (M−H)⁻

### Example 66

### Preparation of Compound 457

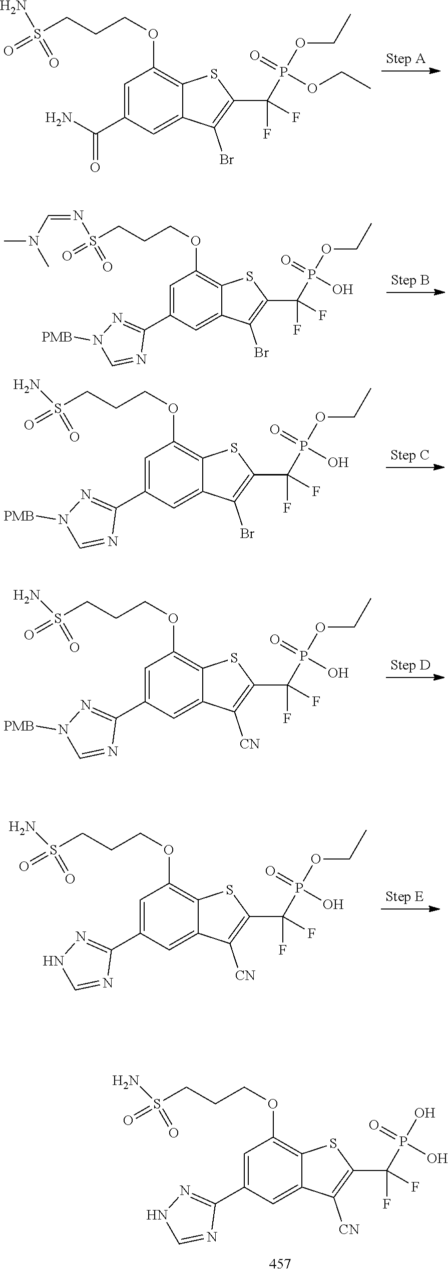

### Step A: Synthesis of ethyl hydrogen (Z)-((3-bromo-7-(3-(N-((dimethylamino)methylene) sulfamoyl)propoxy)-5-(1-(4-methoxybenzyl)-1H-1,2,4-triazol-3-yl)benzo[b]thiophen-2-yl)difluoromethyl)phosphonate

A mixture of diethyl ((3-bromo-5-carbamoyl-7-(3-sulfamoylpropoxy)benzo[b]thiophen-2-yl)difluoromethyl)phosphonate (80 mg, 0.14 mmol) in dimethoxy-N,N-dimethylmethanamine (0.80 mL, 6.0 mmol) was stirred and heated for 2 hours at 80° C. under an argon atmosphere. The reaction mixture was concentrated in vacuo, and the resulting residue was taken up in acetic acid (0.6 mL). To the resulting mixture was added (4-methoxybenzyl)hydrazine hydrochloride (260 mg, 1.38 mmol), and the resulting reaction was heated to 90° C., and was allowed to stir at this temperature for 2 hours. The reaction mixture was then directly purified using reverse phase HPLC (eluting acetonitrile in water) to provide ethyl hydrogen (Z)-((3-bromo-7-(3-(N-((dimethylamino)methylene)sulfamoyl)propoxy)-5-(1-(4-methoxybenzyl)-1H-1,2,4-triazol-3-yl)benzo[b]thiophen-2-yl)difluoromethyl)phosphonate. MS (ESI, m/z): 750, 752 (M+H)⁺

### Step B: Synthesis of ethyl hydrogen ((3-bromo-5-(1-(4-methoxybenzyl)-1H-1,2,4-triazol-3-yl)-7-(3-sulfamoylpropoxy)benzo[b]thiophen-2-yl)difluoromethyl)phosphonate

To a solution of ethyl hydrogen (Z)-((3-bromo-7-(3-(N-((dimethylamino)methylene) sulfamoyl)propoxy)-5-(1-(4-methoxybenzyl)-1H-1,2,4-triazol-3-yl)benzo[b]thiophen-2-yl)difluoromethyl)phosphonate (15 mg, 0.020 mmol) in ethanol (0.2 mL) was added hydrazinium hydroxide (10 μl, 0.20 mmol). The resulting reaction was allowed to stir for 3 hours at room temperature, then the reaction mixture was directly purified using reverse phase HPLC (eluting acetonitrile in water) to provide ethyl hydrogen ((3-bromo-5-(1-(4-methoxybenzyl)-1H-1,2,4-triazol-3-yl)-7-(3-sulfamoylpropoxy)benzo[b]thiophen-2-yl)difluoromethyl)phosphonate. MS (ESI, m/z): 695, 697 (M+H)⁺

### Step C: Synthesis of ethyl hydrogen ((3-cyano-5-(1-(4-methoxybenzyl)-1H-1,2,4-triazol-3-yl)-7-(3-sulfamoylpropoxy)benzo[b]thiophen-2-yl)difluoromethyl)phosphonate

To a mixture of ethyl hydrogen ((3-bromo-5-(1-(4-methoxybenzyl)-1H-1,2,4-triazol-3-yl)-7-(3-sulfamoylpropoxy)benzo[b]thiophen-2-yl)difluoromethyl)phosphonate (60 mg, 0.086 mmol) in N,N-dimethylformamide (1.2 mL) were added tetrakis(triphenylphosphine)palladium (0) (199 mg, 0.173 mmol), and zinc cyanide (20 mg, 0.17 mmol), and put under an argon atmosphere. The resulting reaction was heated to 100° C., and allowed to stir at this temperature for 1.5 hours. The mixture was directly purified using reverse phase HPLC (eluting acetonitrile in water) to provide ethyl hydrogen ((3-cyano-5-(1-(4-methoxybenzyl)-1H-1,2,4-triazol-3-yl)-7-(3-sulfamoylpropoxy)benzo[b]thiophen-2-yl)difluoromethyl)phosphonate. MS (ESI, m/z): 642 (M+H)⁺

### Step D: Synthesis of ethyl hydrogen ((3-cyano-7-(3-sulfamoylpropoxy)-5-(1H-1,2,4-triazol-3-yl)benzo[b]thiophen-2-yl)difluoromethyl)phosphonate

A solution of ethyl hydrogen ((3-cyano-5-(1-(4-methoxybenzyl)-1H-1,2,4-triazol-3-yl)-7-(3-sulfamoylpropoxy)benzo[b]thiophen-2-yl)difluoromethyl)phosphonate (35 mg, 0.055 mmol) in trifluoroacetic acid (0.4 mL) was heated to 80° C., and allowed to stir at this temperature for 30 minutes. The reaction mixture was then directly concentrated in vacuo, and the resulting residue was directly purified using reverse phase HPLC (eluting acetonitrile in water) to provide ethyl hydrogen ((3-cyano-7-(3-sulfamoylpropoxy)-5-(1H-1,2,4-triazol-3-yl)benzo[b]thiophen-2-yl)difluoromethyl)phosphonate. MS (ESI, m/z): 522 (M+H)⁺

### Step E: Synthesis of ((3-cyano-7-(3-sulfamoylpropoxy)-5-(1H-1,2,4-triazol-3-yl)benzo[b]thiophen-2-yl)difluoromethyl)phosphonic acid

To a stirred solution of ethyl hydrogen ((3-cyano-7-(3-sulfamoylpropoxy)-5-(1H-1,2,4-triazol-3-yl)benzo[b]thiophen-2-yl)difluoromethyl)phosphonate (7.0 mg, 0.013 mmol) in N,N-dimethylformamide (100 μL) was added bromotrimethylsilane (52 μL, 0.40 mmol). The resulting reaction was heated to 60° C., and allowed to stir at this temperature for 1.5 hours. The reaction was quenched with ethanol (0.1 mL), concentrated in vacuo, and the resulting residue was purified using reverse phase HPLC [eluting acetonitrile in (water with 10 mM NH₄HCO₃)]to provide ((3-cyano-7-(3-sulfamoylpropoxy)-5-(1H-1,2,4-triazol-3-yl)benzo[b]thiophen-2-yl)difluoromethyl)phosphonic acid (Compound 457). ¹H NMR (400 MHz, D₂O) δ 8.27 (s, 1H), 7.32 (s, 1H), 6.90 (s, 1H), 4.26-4.11 (m, 2H), 3.62-3.45 (m, 2H), 2.51-2.26 (m, 2H). MS (ESI, m/z): 494 (M+H)⁺

### Example 67

### Preparation of Compound 458

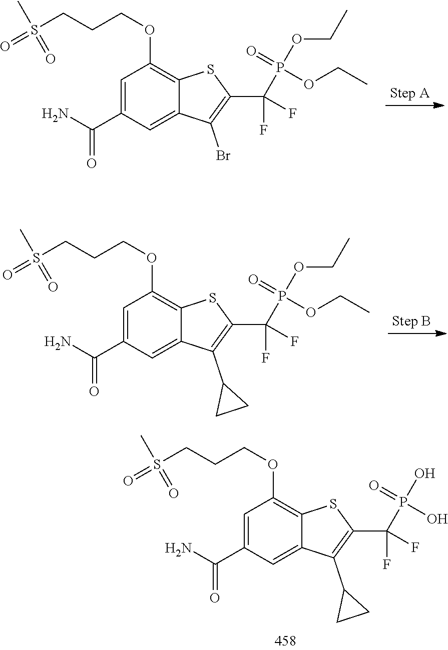

### Step A: Synthesis of diethyl ((5-carbamoyl-3-cyclopropyl-7-(3-(methylsulfonyl)propoxy)benzo[b]thiophen-2-yl)difluoromethyl)phosphonate

To a stirred mixture of diethyl ((3-bromo-5-carbamoyl-7-(3-(methylsulfonyl)propoxy) benzo[b]thiophen-2-yl)difluoromethyl)phosphonate (50 mg, 0.086 mmol) in 2-methyl-2-butanol (1.2 mL) were added cyclopropylboronic acid (9 mg, 0.1 mmol), cesium carbonate (85 mg, 0.26 mmol), and 1,1′-bis (di-t-butylphosphino)ferrocene palladium dichloride (6 mg, 9 μmol). The resulting reaction was heated to 100° C. and allowed to stir at this temperature for 8 hours under an argon atmosphere. The reaction was quenched with water (50 mL), and extracted with ethyl acetate (50 mL×3). The combined organic extracts were dried over anhydrous sodium sulfate, filtered, and concentrated in vacuo. The resulting residue was purified using reverse phase HPLC (eluting acetonitrile in water) to provide diethyl ((5-carbamoyl-3-cyclopropyl-7-(3-(methylsulfonyl)propoxy) benzo[b]thiophen-2-yl)difluoromethyl)phosphonate. MS (ESI, m/z): 540 (M+H)⁺

### Step B: Synthesis of (5-carbamoyl-3-cyclopropyl-7-(3-(methylsulfonyl)propoxy)benzo[b]thiophen-2-yl)difluoromethyl)phosphonic acid

To a stirred solution of diethyl ((5-carbamoyl-3-cyclopropyl-7-(3-(methylsulfonyl)propoxy) benzo[b]thiophen-2-yl)difluoromethyl)phosphonate (10 mg, 0.019 mmol) in DMF (148 μL) was added bromotrimethylsilane (73 μL, 0.56 mmol). The resulting reaction was heated to 50° C. and allowed to stir at this temperature for 2 hours. The reaction was quenched with ethanol (0.2 mL) and concentrated in vacuo. The resulting residue was purified using reverse phase HPLC [eluting acetonitrile in (water with 10 mM NH₄HCO₃)]to provide (5-carbamoyl-3-cyclopropyl-7-(3-(methylsulfonyl)propoxy)benzo[b]thiophen-2-yl)difluoromethyl)phosphonic acid (Compound 457). ¹H NMR (400 MHz, D₂O) δ 8.05 (s, 1H), 7.25 (s, 1H), 4.40 (t, J=5.9 Hz, 2H), 3.55 (t, J=7.8 Hz, 2H), 3.17 (s, 3H), 2.47-2.36 (m, 2H), 2.20-2.09 (m, 1H), 1.16-1.08 (m, 2H), 1.05-0.97 (m, 2H). MS (ESI, m/z): 484 (M+H)⁺

### Example 68

### Preparation of Compound 459

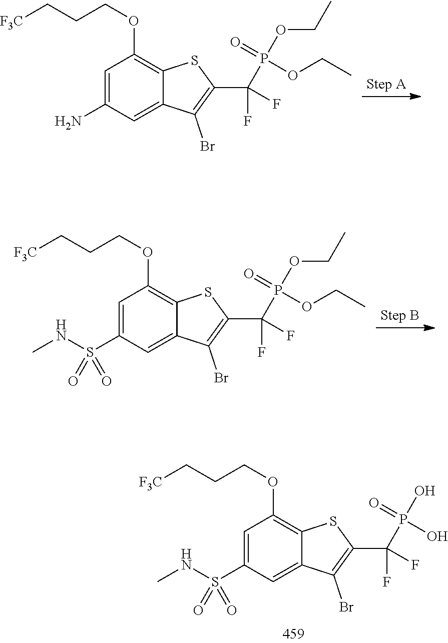 [SMILES: CCOP(=O)(OCC)C(F)(F)c1sc2c(OCCCC(F)(F)F)cc(N)cc2c1Br ; CCOP(=O)(OCC)C(F)(F)c1sc2c(OCCCC(F)(F)F)cc(S(=O)(=O)NC)cc2c1Br ; CNS(=O)(=O)c1cc(OCCCC(F)(F)F)c2sc(C(F)(F)P(=O)(O)O)c(Br)c2c1]

### Step A: Synthesis of diethyl ((3-bromo-5-(N-methylsulfamoyl)-7-(4,4,4-trifluorobutoxy)benzo[b]thiophen-2-yl)difluoromethyl)phosphonate

To a solution of diethyl ((5-amino-3-bromo-7-(4,4,4-trifluorobutoxy)benzo[b]thiophen-2-yl)difluoromethyl)phosphonate (100 mg, 0.185 mmol) in MeCN (430 μL) were added hydrochloric acid (12 M in water, 0.304 mL, 3.7 mmol), and a solution of sodium nitrite (19 mg, 0.28 mmol) in water (130 μL) at 0° C. under an argon atmosphere. The resulting reaction was allowed to stir for 30 minutes at 0° C., then was concentrated in vacuo. The resulting residue was azeotroped with MeCN (3×20 mL) to provide a diazo intermediate. To a suspension of this diazo intermediate in DCE (1 mL) were added 1,4-diazabicyclo[2.2.2]octane-1,4-diium-1,4-disulfinate (45 mg, 0.19 mmol), and 1-hydroxybenzotriazole (30 mg, 0.22 mmol) at 0° C. The resulting reaction was allowed to stir for 1 hour at 0° C., then N,N-diisopropylethylamine (0.039 mL, 0.22 mmol), and methylamine (2.0 M in THF, 1.0 mL, 2.0 mmol). The resulting reaction was warmed to room temperature, and allowed to stir at this temperature for 2 hours. The mixture mixture was then concentrated in vacuo, and the resulting residue was purified using reverse phase HPLC (eluting acetonitrile in water) to provide diethyl ((3-bromo-5-(N-methylsulfamoyl)-7-(4,4,4-trifluorobutoxy)benzo[b]thiophen-2-yl)difluoromethyl)phosphonate. MS (ESI, m/z): 616, 618 (M−H)⁻

### Step B: Synthesis of ((3-bromo-5-(N-methylsulfamoyl)-7-(4,4,4-trifluorobutoxy)benzo[b]thiophen-2-yl)difluoromethyl)phosphonic acid

To a mixture of diethyl ((3-bromo-5-(N-methylsulfamoyl)-7-(4,4,4-trifluorobutoxy)benzo[b]thiophen-2-yl)difluoromethyl)phosphonate (15 mg, 0.024 mmol) in DMF (188 μL) was added bromotrimethylsilane (94 μL, 0.728 mmol). The resulting reaction was heated to 50° C., and allowed to stir at this temperature for 1 hour. The reaction was quenched with ethanol (1 mL), and the resulting solution was directly purified using reverse phase HPLC [eluting acetonitrile in (water with 10 mM NH₄HCO₃)]to provide ((3-bromo-5-(N-methylsulfamoyl)-7-(4,4,4-trifluorobutoxy)benzo[b]thiophen-2-yl)difluoromethyl)phosphonic acid (Compound 459). ¹H NMR (400 MHz, D₂O) δ 7.77 (s, 1H), 7.12 (s, 1H), 4.19 (t, J=6.2 Hz, 2H), 2.41 (s, 3H), 2.38-2.23 (m, 2H), 2.04-1.98 (m, 2H). MS (ESI, m/z): 560, 562 (M−H)⁻

### Example 69

### Preparation of Compound 460

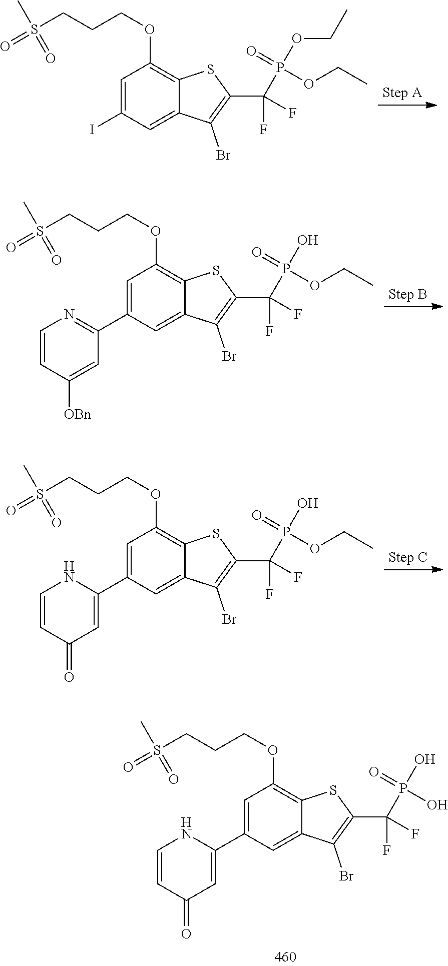 [SMILES: CCOP(=O)(OCC)C(F)(F)c1sc2c(OCCCS(C)(=O)=O)cc(I)cc2c1Br ; CCOP(=O)(O)C(F)(F)c1sc2c(OCCCS(C)(=O)=O)cc(-c3cc(OCc4ccccc4)ccn3)cc2c1Br ; CCOP(=O)(O)C(F)(F)c1sc2c(OCCCS(C)(=O)=O)cc(-c3cc(=O)cc[nH]3)cc2c1Br ; CS(=O)(=O)CCCOc1cc(-c2cc(=O)cc[nH]2)cc2c(Br)c(C(F)(F)P(=O)(O)O)sc12]

### Step A: Synthesis of ethyl hydrogen ((5-(4-(benzyloxy)pyridin-2-yl)-3-bromo-7-(3-(methylsulfonyl) propoxy)benzo[b]thiophen-2-yl)difluoromethyl)phosphonate

To a mixture of diethyl ((3-bromo-5-iodo-7-(3-(methylsulfonyl)propoxy)benzo[b]thiophen-2-yl)difluoromethyl)phosphonate (60 mg, 0.091 mmol) in 1,4-dioxane (0.6 mL) were added 4-(benzyloxy)-2-(trimethylstannyl)pyridine (63 mg, 0.18 mmol), and dichlorobis(triphenylphosphine) palladium (II) (6 mg, 9 μmol). The resulting reaction was heated to 100° C., and allowed to stir at this temperature for 2 hours under an argon atmosphere. The reaction mixture was then directly purified using reverse phase HPLC (eluting acetonitrile in water) to provide ethyl hydrogen ((5-(4-(benzyloxy)pyridin-2-yl)-3-bromo-7-(3-(methylsulfonyl)propoxy)benzo[b]thiophen-2-yl)difluoromethyl)phosphonate. MS (ESI, m/z): 690, 692 (M+H)⁺

### Step B: Synthesis of ethyl hydrogen ((3-bromo-7-(3-(methylsulfonyl)propoxy)-5-(4-oxo-1,4-dihydropyridin-2-yl)benzo[b]thiophen-2-yl)difluoromethyl)phosphonate

To a stirred mixture of ethyl hydrogen ((5-(4-(benzyloxy)pyridin-2-yl)-3-bromo-7-(3-(methylsulfonyl)propoxy)benzo[b]thiophen-2-yl)difluoromethyl)phosphonate (15 mg, 0.021 mmol) in dichloromethane (0.15 mL) was added a solution of trichloroborane in dichloromethane (1.0 M, 0.021 mL, 0.021 mmol) at −78° C. under an argon atmosphere. The resulting reaction was allowed to stir for 2 hours at −78° C. The reaction was then quenched with ethanol (2 mL), and was directly purified using reverse phase HPLC (eluting acetonitrile in water) to provide ethyl hydrogen ((3-bromo-7-(3-(methylsulfonyl)propoxy)-5-(4-oxo-1,4-dihydropyridin-2-yl)benzo[b]thiophen-2-yl)difluoromethyl)phosphonate. MS (ESI, m/z): 600, 602 (M+H)⁺

### Step C: Synthesis of ((3-bromo-7-(3-(methylsulfonyl)propoxy)-5-(4-oxo-1,4-dihydropyridin-2-yl)benzo[b]thiophen-2-yl)difluoromethyl)phosphonic acid

To a stirred solution of ethyl hydrogen ((3-bromo-7-(3-(methylsulfonyl)propoxy)-5-(4-oxo-1,4-dihydropyridin-2-yl)benzo[b]thiophen-2-yl)difluoromethyl)phosphonate (7.0 mg, 0.011 mmol) in DMF (0.1 mL) was added bromotrimethylsilane (0.043 mL, 0.33 mmol). The resulting reaction was heated to 60° C., and allowed to stir at this temperature for 2 hours. The reaction was then quenched with ethanol (0.1 mL), and concentrated in vacuo. The resulting residue was purified using reverse phase HPLC (eluting acetonitrile in water, with 0.1% TFA modifier) to provide ((3-bromo-7-(3-(methylsulfonyl)propoxy)-5-(4-oxo-1,4-dihydropyridin-2-yl)benzo[b]thiophen-2-yl)difluoromethyl)phosphonic acid (Compound 460). ¹H NMR (400 MHz, DMSO-d₆+CD₃CN+CF₃COOD) δ 8.63-8.55 (m, 1H), 8.01-7.96 (m, 1H), 7.77-7.71 (m, 1H), 7.61-7.55 (m, 1H), 7.33-7.27 (m, 1H), 4.49 (t, J=6.1 Hz, 2H), 3.38-3.30 (m, 2H), 3.03 (s, 3H), 2.38-2.26 (m, 2H). MS (ESI, m/z): 572, 574 (M+H)⁺

### Example 70

### Preparation of Compound 461

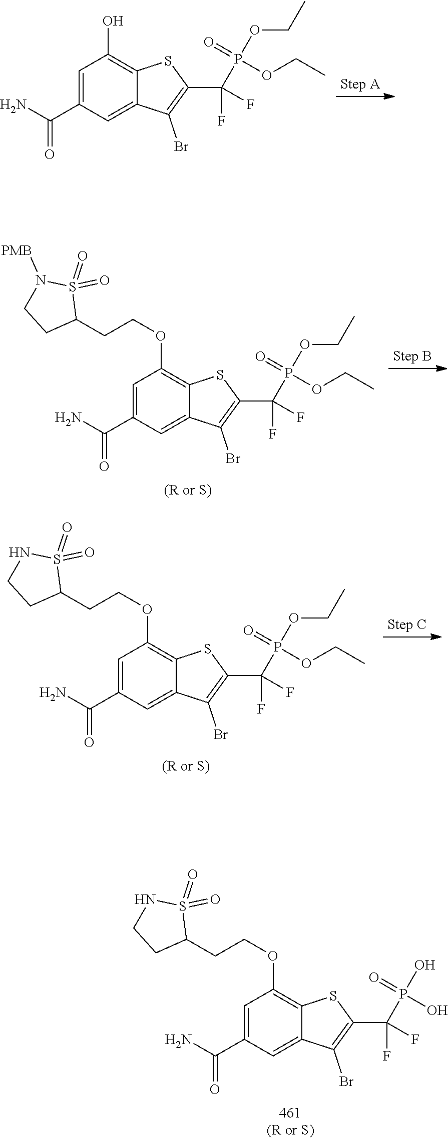

### Step A: Synthesis of diethyl (R or S)-((3-bromo-5-carbamoyl-7-(2-(2-(4-methoxybenzyl)-1,1-dioxidoisothiazolidin-5-yl)ethoxy)benzo[I]thiophen-2-yl)difluoromethyl)phosphonate

To a mixture of diethyl ((3-bromo-5-carbamoyl-7-hydroxybenzo[b]thiophen-2-yl)difluoromethyl)phosphonate (60 mg, 0.13 mmol), and 5-(2-hydroxyethyl)-2-(4-methoxybenzyl)isothiazolidine 1,1-dioxide (37 mg, 0.13 mmol) in toluene (0.6 mL) was added cyanomethylenetributylphosphorane (0.090 mL, 0.33 mmol). The resulting reaction was heated to 80° C., and allowed to stir at this temperature for 2 hours. The reaction was quenched with water (20 mL), and extracted with ethyl acetate (50 mL×3). The combined organic extracts were washed with brine (20 mL×3), dried over anhydrous sodium sulfate, filtered, and concentrated in vacuo. The resulting residue was purified using silica gel chromatography (eluting methanol in dichloromethane) followed by purification by reverse phase HPLC (eluting acetonitrile in water) to provide the product as a racemic mixture. The product was resolved using chiral HPLC {Column: (R, R)-WHELK-01-Kromasil, 5×25 cm, 5 μm; eluting 15% [tert-butyl methyl ether (with 0.5% 2M NH₃-MeOH)]in ethanol; Flow rate: 20 mL/min} to provide diethyl (R or S)-((3-bromo-5-carbamoyl-7-(2-(2-(4-methoxybenzyl)-1,1-dioxidoisothiazolidin-5-yl)ethoxy)benzo[b]thiophen-2-yl)difluoromethyl)phosphonate as the first eluting peak (t_{R}=10.6 min), and diethyl (S or R)-((3-bromo-5-carbamoyl-7-(2-(2-(4-methoxybenzyl)-1,1-dioxidoisothiazolidin-5-yl)ethoxy)benzo[b]thiophen-2-yl)difluoromethyl)phosphonate as the second eluting peak (t_{R}=13.5 min). MS (ESI, m/z): 725, 727 (M+H)⁺

### Step B: Synthesis of diethyl (R or S)-((3-bromo-5-carbamoyl-7-(2-(1,1-dioxidoisothiazolidin-5-yl)ethoxy)benzo[b]thiophen-2-yl)difluoromethyl)phosphonate

A mixture of diethyl (R or S)-((3-bromo-5-carbamoyl-7-(2-(2-(4-methoxybenzyl)-1,1-dioxidoisothiazolidin-5-yl)ethoxy)benzo[b]thiophen-2-yl)difluoromethyl)phosphonate (15 mg, 0.021 mmol) in TFA (0.1 mL) was heated to 60° C., and allowed to stir at this temperature for 1 hour. The reaction mixture was concentrated in vacuo to provide diethyl (R or S)-((3-bromo-5-carbamoyl-7-(2-(1,1-dioxidoisothiazolidin-5-yl)ethoxy)benzo[b]thiophen-2-yl)difluoromethyl)phosphonate, which was used in the next step without purification. MS (ESI, m/z): 605, 607 (M+H)⁺

### Step C: Synthesis of (R or S)-((3-bromo-5-carbamoyl-7-(2-(1,1-dioxidoisothiazolidin-5-yl)ethoxy)benzo[b]thiophen-2-yl)difluoromethyl)phosphonic acid

To a stirred solution of diethyl (R or S)-((3-bromo-5-carbamoyl-7-(2-(1,1-dioxidoisothiazolidin-5-yl)ethoxy)benzo[b]thiophen-2-yl)difluoromethyl)phosphonate (8.0 mg, 0.013 mmol) in DMF (104 μL) was added bromotrimethylsilane (44 μL, 0.33 mmol). The resulting reaction was heated to 60° C., and allowed to stir at this temperature for 1.5 hours. The reaction was quenched with ethanol (0.5 mL), and concentrated in vacuo. The resulting residue was purified using reverse phase HPLC [eluting acetonitrile in (water with 10 mM NH₄HCO₃)]to provide (R or S)-((3-bromo-5-carbamoyl-7-(2-(1,1-dioxidoisothiazolidin-5-yl)ethoxy)benzo[b]thiophen-2-yl)difluoromethyl)phosphonic acid (Compound 461). ¹H NMR (400 MHz, D₂O) δ 7.73 (s, 1H), 7.14 (s, 1H), 4.33 (t, J=6.0 Hz, 2H), 3.62-3.52 (m, 1H), 3.41-3.34 (m, 2H), 2.79-2.67 (m, 1H), 2.49-2.38 (m, 1H), 2.30-2.16 (m, 2H). MS (ESI, m/z): 549, 551 (M+H)⁺

The following illustrative compounds were made using the methods described in the Example immediately above, utilizing the other stereoisomer obtained from the chiral separation following Step A.

| Com- | | Mass |
|---|---|---|
| pound | Structure | [M + H]⁺ |
| 462 | 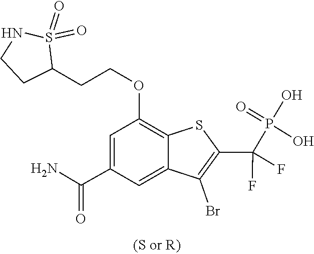 [SMILES: NC(=O)c1cc(OCCC2CCNS2(=O)=O)c2sc(C(F)(F)P(=O)(O)O)c(Br)c2c1] | 549, 551 |

### Example 70

### Preparation of Compound 463

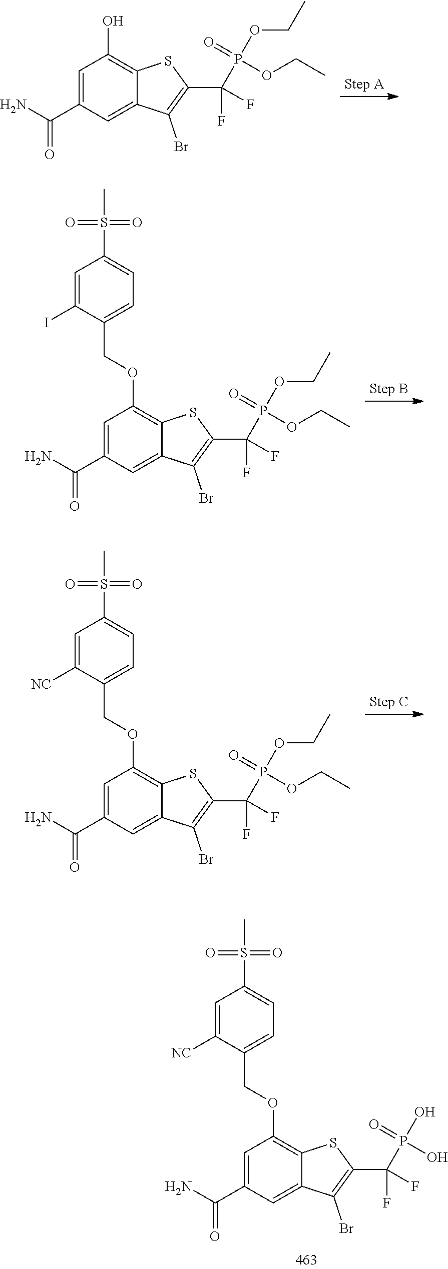 [SMILES: CCOP(=O)(OCC)C(F)(F)c1sc2c(O)cc(C(N)=O)cc2c1Br ; CCOP(=O)(OCC)C(F)(F)c1sc2c(OCc3ccc(S(C)(=O)=O)cc3I)cc(C(N)=O)cc2c1Br ; CCOP(=O)(OCC)C(F)(F)c1sc2c(OCc3ccc(S(C)(=O)=O)cc3C#N)cc(C(N)=O)cc2c1Br ; CS(=O)(=O)c1ccc(COc2cc(C(N)=O)cc3c(Br)c(C(F)(F)P(=O)(O)O)sc23)c(C#N)c1]

### Step A: Synthesis of diethyl ((3-bromo-5-carbamoyl-7-((2-iodo-4-(methylsulfonyl)benzyl)oxy) benzo[b]thiophen-2-yl)difluoromethyl)phosphonate

Diisopropyl azodicarboxylate (0.034 mL, 0.18 mmol) was added to a mixture of diethyl ((3-bromo-5-carbamoyl-7-hydroxybenzo[b]thiophen-2-yl)difluoromethyl)phosphonate (40 mg, 0.087 mmol), (2-iodo-4-(methylsulfonyl)phenyl)methanol (55 mg, 0.18 mmol), and triphenylphosphane (46 mg, 0.18 mmol) in THF (0.5 mL) at 0° C. The resulting reaction was warmed to room temperature, and allowed to stir at this temperature for 2 hours. The reaction mixture was then directly purified using reverse phase HPLC (eluting acetonitrile in water) to provide diethyl ((3-bromo-5-carbamoyl-7-((2-iodo-4-(methylsulfonyl)benzyl)oxy)benzo[b]thiophen-2-yl)difluoromethyl)phosphonate. ¹H NMR (400 MHz, CDCl₃) δ 8.44 (d, J=1.8 Hz, 1H), 8.00-7.96 (m, 1H), 7.91 (d, J=1.3 Hz, 1H), 7.75 (d, J=8.1 Hz, 1H), 7.57 (s, 1H), 5.34 (s, 2H), 4.39-4.23 (m, 4H), 3.10 (s, 3H), 1.42-1.36 (m, 6H). MS (ESI, m/z): 752, 754 (M+H)⁺

### Step B: Synthesis of diethyl ((3-bromo-5-carbamoyl-7-((2-cyano-4-(methylsulfonyl)benzyl)oxy) benzo[b]thiophen-2-yl)difluoromethyl)phosphonate

A mixture of diethyl ((3-bromo-5-carbamoyl-7-((2-iodo-4-(methylsulfonyl)benzyl)oxy) benzo[b]thiophen-2-yl)difluoromethyl)phosphonate (30 mg, 0.040 mmol), dicyanozinc (5 mg, 0.04 mmol), and tetrakis(triphenylphosphine)palladium (9 mg, 8 μmol) in DMF (0.3 mL) was allowed to stir at 100° C. for 30 minutes under an argon atmosphere. The reaction mixture was cooled to room temperature, and directly purified using reverse phase HPLC (eluting acetonitrile in water) to provide diethyl ((5-carbamoyl-3-cyano-7-((2-cyano-4-(methylsulfonyl)benzyl)oxy)benzo[b]thiophen-2-yl)difluoromethyl)phosphonate. ¹H NMR (400 MHz, CDCl₃) δ 8.23 (s, 1H), 8.17-8.12 (m, 1H), 7.99 (s, 1H), 7.91 (d, J=8.2 Hz, 1H), 7.50 (s, 1H), 5.52 (s, 2H), 4.26-4.16 (m, 4H), 3.04 (s, 3H), 1.32-1.26 (m, 6H). MS (ESI, m/z): 651, 653 (M+H)⁺

### Step C: Synthesis of ((3-bromo-5-carbamoyl-7-((2-cyano-4-(methylsulfonyl)benzyl)oxy)benzo[b]thiophen-2-yl)difluoromethyl)phosphonic acid

Bromotrimethylsilane (91 μL, 0.69 mmol) was added to a mixture of diethyl ((3-bromo-5-carbamoyl-7-((2-cyano-4-(methylsulfonyl)benzyl)oxy)benzo[b]thiophen-2-yl)difluoromethyl) phosphonate (15 mg, 0.023 mmol) in DMF (182 μL) at room temperature under an argon atmosphere. The resulting reaction was heated to 60° C., and allowed to stir at this temperature for 1 hour. The reaction was quenched with ethanol (0.5 mL), and concentrated in vacuo. The resulting residue was purified using reverse phase HPLC [eluting acetonitrile in (water with 0.01 M ammonium bicarbonate)]to provide ((3-bromo-5-carbamoyl-7-((2-cyano-4-(methylsulfonyl)benzyl) oxy)benzo[b]thiophen-2-yl)difluoromethyl)phosphonic acid (Compound 463). ¹H NMR (400 MHz, D₂O) δ 8.60 (d, J=2.0 Hz, 1H), 8.42 (dd, J=8.2, 2.0 Hz, 1H), 8.22-8.18 (m, 2H), 7.69 (d, J=1.3 Hz, 1H), 5.76 (s, 2H), 3.44 (s, 3H). MS (ESI, m/z): 595, 597 (M+H)⁺

### Example 71

### Preparation of Compound 464

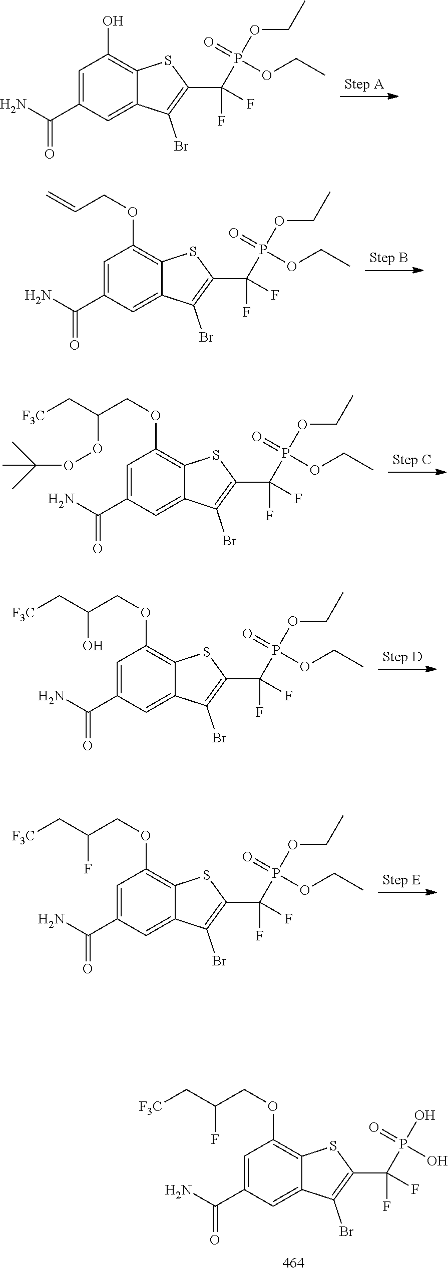

### Step A: Synthesis of diethyl ((7-(allyloxy)-3-bromo-5-carbamoylbenzo[b]thiophen-2-yl) difluoromethyl)phosphonate

A mixture of diethyl ((3-bromo-5-carbamoyl-7-hydroxybenzo[b]thiophen-2-yl)difluoromethyl)phosphonate (500 mg, 1.09 mmol), potassium carbonate (302 mg, 2.18 mmol), and 3-bromoprop-1-ene (198 mg, 1.64 mmol) in DMF (5 mL) was heated to 50° C., and allowed to stir for 1 hour. The mixture was directly purified using reverse phase HPLC (eluting acetonitrile in water) to provide diethyl ((7-(allyloxy)-3-bromo-5-carbamoylbenzo[b]thiophen-2-yl)difluoromethyl)phosphonate. ¹H NMR (400 MHz, CDCl₃) δ 7.84 (s, 1H), 7.46 (s, 1H), 6.17-6.01 (m, 1H), 5.56-5.43 (m, 1H), 5.39-5.31 (m, 1H), 4.80 (d, J=5.2 Hz, 2H), 4.39-4.20 (m, 4H), 1.38 (t, J=7.1 Hz, 6H). MS (ESI, m/z): 498, 500 (M+H)⁺

### Step B: Synthesis of diethyl ((3-bromo-7-(2-(tert-butylperoxy)-4,4,4-trifluorobutoxy)-5-carbamoylbenzo[b]thiophen-2-yl)difluoromethyl)phosphonate

tert-Butyl hydroperoxide (70% in water, 1008 mg, 7.83 mmol) was added to a mixture of diethyl ((7-(allyloxy)-3-bromo-5-carbamoylbenzo[b]thiophen-2-yl)difluoromethyl)phosphonate (600 mg, 1.20 mmol), sodium trifluoromethanesulfinate (376 mg, 2.41 mmol), cobalt (II) acetate tetrahydrate (30 mg, 0.12 mmol), and potassium phosphate tribasic (153 mg, 0.722 mmol) in acetonitrile (6 mL). The mixture was heated to 75° C., and allowed to stir for 2 hours. The mixture was directly purified using reverse phase HPLC (eluting acetonitrile in water) to provide diethyl ((3-bromo-7-(2-(tert-butylperoxy)-4,4,4-trifluorobutoxy)-5-carbamoylbenzo[b]thiophen-2-yl)difluoromethyl)phosphonate. ¹H NMR (400 MHz, CDCl₃) δ 7.88 (s, 1H), 7.54 (s, 1H), 4.66-4.58 (m, 1H), 4.55-4.46 (m, 1H), 4.44-4.37 (m, 1H), 4.35-4.27 (m, 4H), 2.69-2.53 (m, 2H), 1.38 (t, J=7.1, 0.6 Hz, 6H), 1.25 (s, 9H). MS (ESI, m/z): 656, 658 (M+H)⁺

### Step C: Synthesis of diethyl ((3-bromo-5-carbamoyl-7-(4,4,4-trifluoro-2-hydroxybutoxy)benzo[b]thiophen-2-yl)difluoromethyl)phosphonate

Zinc (120 mg, 1.83 mmol) was added to a mixture of diethyl ((3-bromo-7-(2-(tert-butylperoxy)-4,4,4-trifluorobutoxy)-5-carbamoylbenzo[b]thiophen-2-yl)difluoromethyl)phosphonate (120 mg, 0.183 mmol) in acetic acid (1.2 mL) under an argon atmosphere. The mixture was heated to 40° C., and allowed to stir for 16 hours. The mixture was filtered and the filtrate was directly purified using reverse phase HPLC (eluting acetonitrile in water) to provide diethyl ((3-bromo-5-carbamoyl-7-(4,4,4-trifluoro-2-hydroxybutoxy)benzo[b]thiophen-2-yl)difluoromethyl)phosphonate. ¹H NMR (400 MHz, CDCl₃) δ 7.84 (s, 1H), 7.47 (s, 1H), 4.53-4.43 (m, 1H), 4.41-4.21 (m, 6H), 2.61-2.46 (m, 2H), 1.39 (t, J=7.0 Hz, 6H). MS (ESI, m/z): 584, 586 (M+H)⁺

### Step D: Synthesis of diethyl ((3-bromo-5-carbamoyl-7-(2,4,4,4-tetrafluorobutoxy)benzo[b]thiophen-2-yl)difluoromethyl)phosphonate

Diethylaminosulfur trifluoride (0.018 mL, 0.14 mmol) was added to a mixture of diethyl ((3-bromo-5-carbamoyl-7-(4,4,4-trifluoro-2-hydroxybutoxy)benzo[b]thiophen-2-yl)difluoromethyl)phosphonate (40 mg, 0.068 mmol) in DCM (0.4 mL) at 0° C. under an argon atmosphere. The mixture was warmed to room temperature and stirred for 1 hour. The reaction was quenched with saturated aqueous sodium bicarbonate (20 mL), and extracted with ethyl acetate (30 mL×3). The combined organic extracts were washed with brine (30 mL×3), dried over anhydrous sodium sulfate, filtered, and concentrated in vacuo. The resulting residue was directly purified using reverse phase HPLC (eluting acetonitrile in water) to provide diethyl ((3-bromo-5-carbamoyl-7-(2,4,4,4-tetrafluorobutoxy)benzo[b]thiophen-2-yl)difluoromethyl)phosphonate. ¹H NMR (400 MHz, CDCl₃) δ 7.88 (s, 1H), 7.49 (s, 1H), 5.33-5.13 (m, 1H), 4.58-4.20 (m, 6H), 2.90-2.54 (m, 2H), 1.39 (t, J=7.0 Hz, 6H). MS (ESI, m/z): 586, 588 (M+H)⁺

### Step E: Synthesis of ((3-bromo-5-carbamoyl-7-(2,4,4,4-tetrafluorobutoxy)benzo[b]thiophen-2-yl)difluoromethyl)phosphonic acid

Bromotrimethylsilane (0.066 mL, 0.51 mmol) was added to a mixture of diethyl ((3-bromo-5-carbamoyl-7-(2,4,4,4-tetrafluorobutoxy)benzo[b]thiophen-2-yl)difluoromethyl)phosphonate (10 mg, 0.017 mmol) in DMF (0.15 mL) under an argon atmosphere. The mixture was heated to 60° C., and allowed to stir for 1 hour. The reaction was quenched with ethanol (0.5 mL), and concentrated in vacuo. The resulting residue was purified using reverse phase HPLC [eluting acetonitrile in (water with 0.01 M ammonium bicarbonate)]to provide ((3-bromo-5-carbamoyl-7-(2,4,4,4-tetrafluorobutoxy)benzo[b]thiophen-2-yl)difluoromethyl)phosphonic acid (Compound 464). ¹H NMR (400 MHz, D₂O) δ 7.71 (s, 1H), 7.13 (s, 1H), 5.51-5.29 (m, 1H), 4.56-4.29 (m, 2H), 3.09-2.67 (m, 2H). MS (ESI, m/z): 530, 532 (M+H)⁺

### Example 72

### Human PTPN2 Biochemical Assay

Test compounds were dissolved in DMSO, and 10-point serial 3-fold dilution series in DMSO were prepared in Echo Qualified 384-well Polypropylene Microplates (Labcyte, San Jose, CA) (top concentration 50 M). Assay plates (384-well low volume black plates; Corning #3820, Corning, NY) were prepared by dispensing 50 nL of test compound, and DMSO (for high and low controls) by ECHO acoustic dispenser (Labcyte, San Jose CA). This was followed by addition of 5 μL of 0.2 nM human PTPN2 (1-387) solution (prepared in the assay buffer, 50 mM Tris pH 7.4, 150 mM NaCl, 0.01% Tween20, 0.5 mM dithiothreitol) to all wells except the low control. 5 μl of assay buffer was added into the low control, then the plate was incubated for 30 minutes at room temperature. Subsequently, 5 μl of 100 M DiFMUP (6,8-difluoro-4-methylumbelliferyl phosphate) solution (prepared in assay buffer from 10 mM stock in DMSO) was added to the assay plate and incubated for 1 hour at room temperature. For detection, assay plates were read on SpectraMax Microplate Reader (Molecular Devices), with Excitation wavelength=360 nm and Emission wavelength=460 nm. Test compound effects were normalized to the window defined by the controls, DMSO/buffer, and DMSO/50 pM human PTPN2. Calculated % effects were fit using a 4-parameter algorithm, and EC₅₀ was reported.

### Example 73

### Human PTPN1 Biochemical Assay

Test compounds were dissolved in DMSO, and 10-point serial 3-fold dilution series in DMSO were prepared in Echo Qualified 384-well Polypropylene Microplates (Labcyte, San Jose, CA) (top concentration 50 M). Assay plates (384-well low volume black plates; Corning #3820, Corning, NY) were prepared by dispensing 50 nL of test compound and DMSO (for high and low controls) by ECHO acoustic dispenser (Labcyte, San Jose CA). This was followed by addition of 5 μL of 6 nM human PTPN1 (1-435) solution (prepared in the assay buffer, 50 mM Tris pH 7.4, 150 mM NaCl, 0.01% Tween20, 0.5 mM dithiothreitol) to all wells except the low control. 5 μl of assay buffer was added into the low control. The plate was incubated for 30 minutes at room temperature. Subsequently, 5 μl of 100 M DiFMUP (6,8-difluoro-4-methylumbelliferyl phosphate) solution (prepared in assay buffer from 10 mM stock in DMSO) was added to the assay plate and incubated for 1 hour at room temperature. For detection, assay plates were read on SpectraMax Microplate Reader (Molecular Devices), with Excitation wavelength=360 nm and Emission wavelength=460 nm. Test compound effects were normalized to the window defined by the controls, DMSO/buffer, and DMSO/150 pM human PTPN1. Calculated % effects were fit using a 4-parameter algorithm, and EC₅₀ was reported.

Illustrative compounds of the present disclosure were tested in one or more of the above assays and results are provided in the table below:

| Compound | PTPN2 IC₅₀ (nM) | PTP1B IC₅₀ (nM) |
|---|---|---|
| 1 | 3007 | 1608 |
| 2 | 5324 | 3233 |
| 3 | 1024 | 1126 |
| 4 | 1199 | 1445 |
| 5 | 896.5 | 1129 |
| 6 | 4643 | 6048 |
| 7 | 1329 | 764 |
| 8 | 3937 | 3271 |
| 9 | 2129 | 2199 |
| 10 | 5.791 | 4.609 |
| 11 | 3053 | 2162 |
| 12 | 8891 | 5247 |
| 13 | 5304 | 4113 |
| 14 | 4123 | 2693 |
| 15 | 6162 | 5624 |
| 16 | 7254 | 5120 |
| 17 | 1913 | 1456 |
| 18 | 2169 | 1334 |
| 19 | 2351 | 1497 |
| 20 | 6137 | 4441 |
| 21 | 2145 | 1413 |
| 22 | 2588 | 1988 |
| 23 | 9282 | 7253 |
| 24 | 2162 | 1362 |
| 25 | 3165 | 2571 |
| 26 | 2341 | 1718 |
| 27 | 3232 | 2187 |
| 28 | 6932 | 5158 |
| 29 | 3340 | 2490 |
| 30 | 2093 | 1836 |
| 31 | 1465 | 1355 |
| 32 | 1885 | 1312 |
| 33 | 1865 | 2119 |
| 34 | 5862 | 4477 |
| 35 | 7159 | 6435 |
| 36 | 3925 | 2752 |
| 37 | 3488 | 2648 |
| 38 | 4238 | 3395 |
| 39 | 2996 | 2271 |
| 40 | 5775 | 4195 |
| 41 | 3230 | 2229 |
| 42 | 2376 | 1782 |
| 43 | 6527 | 4899 |
| 44 | 3060 | 2467 |
| 45 | 5167 | 4075 |
| 46 | 8502 | 6228 |
| 47 | 4913 | 4214 |
| 48 | 7151 | 5534 |
| 49 | 5091 | 4148 |
| 50 | 7487 | 6392 |
| 51 | 1351 | 1113 |
| 52 | 6818 | 5681 |
| 53 | 4108 | 3237 |
| 54 | 6753 | 5031 |
| 55 | 9695 | 7618 |
| 56 | 3931 | 2881 |
| 57 | 5092 | 4332 |
| 58 | 7904 | 9053 |
| 59 | 550 | 1387 |
| 60 | 2533 | 5716 |
| 61 | 904.1 | 638.3 |
| 62 | 1612 | 1997 |
| 63 | 542.1 | 443.6 |
| 64 | 1798 | 1440 |
| 65 | 4853 | 4305 |
| 66 | 719.4 | 515.6 |
| 67 | 4889 | 3180 |
| 68 | 4243 | 3773 |
| 69 | 4157 | 6254 |
| 70 | 8750 | 9209 |
| 71 | 3530 | 3650 |
| 72 | 3522 | 2753 |
| 73 | 3082 | 2555 |
| 74 | 7440 | 5763 |
| 75 | 2704 | 1849 |
| 76 | 958.3 | 1883 |
| 77 | 1112 | 730 |
| 78 | 1029 | 1086 |
| 79 | 9752 | 8095 |
| 80 | 1887 | 2128 |
| 81 | 27.32 | 114.5 |
| 82 | 1883 | 1390 |
| 83 | 2009 | 1631 |
| 84 | 3474 | 7431 |
| 85 | 4541 | 4666 |
| 86 | 1447 | 3781 |
| 87 | 3301 | 7092 |
| 88 | 1512 | 1093 |
| 89 | 6035 | 5125 |
| 90 | 5803 | 4205 |
| 91 | 6170 | 4981 |
| 92 | 3160 | 1938 |
| 93 | 4329 | 4133 |
| 94 | 2800 | 2404 |
| 95 | 4784 | 3895 |
| 96 | 1279 | 939.8 |
| 97 | 8234 | 7079 |
| 98 | 4729 | 4567 |
| 99 | 3791 | 2616 |
| 100 | 682 | 539.9 |
| 101 | 2356 | 1783 |
| 102 | 2457 | 2878 |
| 103 | 3460 | 3142 |
| 104 | 6497 | 9147 |
| 105 | 5777 | 5139 |
| 106 | 8969 | 6915 |
| 107 | 4034 | 3858 |
| 108 | 3620 | 4077 |
| 109 | 779.8 | 465.1 |
| 110 | 3557 | 3202 |
| 111 | 4260 | 3982 |
| 112 | 2906 | 3683 |
| 113 | 4167 | 4082 |
| 114 | 5167 | 3496 |
| 115 | 2604 | 3616 |
| 116 | 1063 | 986.5 |
| 117 | 2729 | 2136 |
| 118 | 6172 | 5907 |
| 119 | 629.6 | 404.1 |
| 120 | 4993 | 3795 |
| 121 | 3104 | 2307 |
| 122 | 5370 | 3886 |
| 123 | 4294 | 3558 |
| 124 | 7375 | 6125 |
| 125 | 6520 | 5228 |
| 126 | 6111 | 5226 |
| 127 | 6698 | 5143 |
| 128 | 4904 | 4075 |
| 129 | 2645 | 2228 |
| 130 | 1216 | 1094 |
| 131 | 4164 | 4130 |
| 132 | 5230 | 4703 |
| 133 | 1182 | 1447 |
| 134 | 2884 | 2399 |
| 135 | 1899 | 1924 |
| 136 | 4253 | 3758 |
| 137 | 1422 | 1094 |
| 138 | 2914 | 2084 |
| 139 | 1488 | 1299 |
| 140 | 1813 | 1476 |
| 141 | 3716 | 2316 |
| 142 | 1870 | 2640 |
| 143 | 6136 | 4341 |
| 144 | 7174 | 1414 |
| 145 | 6975 | 4590 |
| 146 | 7474 | 3654 |
| 147 | 5661 | 3189 |
| 148 | 5943 | 5271 |
| 149 | 6011 | 3129 |
| 150 | 6279 | 2589 |
| 151 | 5975 | 2737 |
| 152 | 8218 | 4414 |
| 153 | 1952 | 3158 |
| 154 | 2770 | 1520 |
| 155 | 2164 | 2378 |
| 156 | 902.3 | 1787 |
| 157 | 5252 | 2376 |
| 158 | 455.9 | 1452 |
| 159 | 469.9 | 250.6 |
| 160 | 545.8 | 314.9 |
| 161 | 344 | 445.9 |
| 162 | 221.7 | 243.6 |
| 163 | 301.3 | 436.4 |
| 164 | 435.4 | 361.4 |
| 165 | 389.7 | 675.4 |
| 166 | 3605 | 3163 |
| 167 | 3070 | 3557 |
| 168 | 29.67 | 28.24 |
| 169 | 132.9 | 115.4 |
| 170 | 635.5 | 518.4 |
| 171 | 1466 | 2184 |
| 172 | 580.7 | 662.6 |
| 173 | 577.5 | 738.5 |
| 174 | 4137 | 8606 |
| 175 | 2022 | 1418 |
| 176 | 4720 | 2680 |
| 177 | 2071 | 839 |
| 178 | 5822 | 10840 |
| 179 | 2571 | 2839 |
| 180 | 6633 | 13730 |
| 181 | 4101 | 2416 |
| 182 | 5078 | 2654 |
| 183 | 1996 | 1409 |
| 184 | 7345 | 4968 |
| 185 | 5202 | 3388 |
| 186 | 647.7 | 1384 |
| 187 | 1864 | 961 |
| 188 | 1409 | 714 |
| 189 | 1320 | 990 |
| 190 | 1829 | 1018 |
| 191 | 2825 | 3597 |
| 192 | 7982 | 4592 |
| 193 | 5835 | 3143 |
| 194 | 3195 | 1741 |
| 195 | 5544 | 2741 |
| 196 | 3543 | 2096 |
| 197 | 9459 | 7209 |
| 198 | 5459 | 5708 |
| 199 | 1713 | 1189 |
| 200 | 1469 | 976 |
| 201 | 9.056 | 10.9 |
| 202 | 3649 | 2316 |
| 203 | 16.39 | 17.2 |
| 204 | 1259 | 1430 |
| 205 | 6.949 | 7.5 |
| 206 | 8.039 | 9 |
| 207 | 9.791 | 11.6 |
| 208 | 6.959 | 7.5 |
| 209 | 4.626 | 6.5 |
| 210 | 13.68 | 131.9 |
| 211 | 511.4 | 696 |
| 212 | 9662 | 8152 |
| 213 | 6357 | 5291 |
| 214 | 4325 | 3480 |
| 215 | 8429 | 7551 |
| 216 | 5757 | 6409 |
| 217 | 7057 | 1845 |
| 218 | 2046 | 2903 |
| 219 | 3736 | 4655 |
| 220 | 6062 | 7138 |
| 221 | 6679 | 3455 |
| 222a | 1884 | 1623 |
| 222b | 770.6 | 707.1 |
| 223 | 3209 | 2440 |
| 224 | 1609 | 2389 |
| 225 | 2196 | 2410 |
| 226 | 2694 | 2030 |
| 227 | 855.8 | 1001 |
| 228 | 1606 | 1854 |
| 229 | 86.21 | 531.3 |
| 230 | 1729 | 1030 |
| 231 | 988.7 | 878.6 |
| 232 | 3.059 | 3.4 |
| 233 | 145.7 | 77 |
| 234 | 773.5 | 490 |
| 235 | 16.59 | 20 |
| 236 | 13.49 | 15.2 |
| 237 | 440.8 | 543.8 |
| 238 | 227 | 327.3 |
| 239 | 53.87 | 55.4 |
| 240 | 98.72 | 112.5 |
| 241 | 50.48 | 61.8 |
| 242 | 60 | 79.6 |
| 243 | 47.89 | 68.4 |
| 244 | 227 | 259.1 |
| 245 | 1464 | 903 |
| 246 | 1171 | N/A |
| 247 | 54.42 | 94.2 |
| 248 | 49.96 | 54 |
| 249 | 60.89 | 84.5 |
| 250 | 54.11 | 69.7 |
| 251 | 271.3 | 422.7 |
| 252 | 106.2 | 109.8 |
| 253 | 84.62 | 118.1 |
| 254 | 111.2 | 154.8 |
| 255 | 96.36 | 139.1 |
| 256 | 128.2 | 66 |
| 257 | 293.9 | 237 |
| 258 | 8.395 | 4.3 |
| 259 | 338.1 | 180 |
| 260 | 15.7 | 11.4 |
| 261 | 5878 | 2019 |
| 262 | 112.5 | 42 |
| 263 | 650.6 | 386 |
| 264 | 26.32 | 24 |
| 265 | 60.34 | 62 |
| 266 | 12.88 | 17.4 |
| 267 | 12.23 | 26 |
| 268 | 19.98 | 15.69 |
| 269 | 26.44 | 37.1 |
| 270 | 21.57 | 23.3 |
| 271 | 338.3 | 399.8 |
| 272 | 47.21 | 35 |
| 273 | 131.7 | 93 |
| 274 | 64.94 | 43 |
| 275 | 175.7 | 83 |
| 276 | 27.3 | 17 |
| 277 | 107.8 | 69 |
| 278 | 17.4 | 16 |
| 279 | 230.8 | 97 |
| 280 | 99.4 | 62 |
| 281 | 27.8 | 18 |
| 282 | 167.6 | 71 |
| 283 | 50.2 | 28 |
| 284 | 64.5 | 42 |
| 285 | 77.9 | 45 |
| 286 | 76.9 | 40 |
| 287 | 45.3 | 16 |
| 288 | 6.856 | 7.3 |
| 289 | 63.17 | 53.6 |
| 290 | 9.74 | 11 |
| 291 | 26.82 | 21 |
| 292 | 78.6 | 49 |
| 293 | 618.9 | 246 |
| 294 | 80.7 | 38 |
| 295 | 38.88 | 38 |
| 296 | 241.4 | N/A |
| 297 | 26480 | 7350 |
| 298 | 21560 | 11730 |
| 299 | 17090 | 8984 |
| 300 | 16920 | 7327 |
| 301 | 17050 | 12930 |
| 302 | 43680 | 37930 |
| 303 | 24740 | 2691 |
| 304 | 8.898 | 9.8 |
| 305 | 7.604 | 8.6 |
| 306 | 16.4 | 25.7 |
| 307 | 64.98 | 57.5 |
| 308 | 25.24 | 23.4 |
| 309 | 335.2 | N/A |
| 310 | 68.5 | 77.7 |
| 311 | 120 | 140 |
| 312 | 71.2 | 79.1 |
| 313 | 131.8 | 146 |
| 314 | 52.6 | 47.5 |
| 315 | 21.8 | 23.4 |
| 316 | 50.4 | 68.1 |
| 317 | 68.5 | 68.4 |
| 318 | 34.8 | 52.6 |
| 319 | 9.669 | 9.2 |
| 320 | 6.812 | 7.5 |
| 321 | 24.09 | 25.1 |
| 322 | 29.25 | 20.9 |
| 323 | 55.44 | 44 |
| 324 | 29.88 | 41 |
| 325 | 385 | 182.8 |
| 326 | 16.1 | 2.5 |
| 327 | 265.8 | 233 |
| 328 | 7.835 | 7.9 |
| 329 | 14.1 | 18.1 |
| 330 | 10.45 | 9.2 |
| 331 | 23.78 | 28.8 |
| 332 | 28.14 | 29.9 |
| 333 | 42.08 | 37 |
| 334 | 77.66 | 65.77 |
| 335 | 4.4 | 5.4 |
| 336 | 88.6 | 160 |
| 337 | 83.3 | 158 |
| 338 | 51.2 | 74.4 |
| 339 | 24.4 | 45.3 |
| 340 | 115.5 | 283 |
| 341 | 46.7 | 94.3 |
| 342 | 75.1 | 131 |
| 343 | 19.3 | 128 |
| 344 | 116.7 | 195 |
| 345 | 16.3 | 29.5 |
| 346 | 2.5 | 16.4 |
| 347 | 23.8 | 36.5 |
| 348 | 75.7 | 177 |
| 349 | 22 | 26.9 |
| 350 | 159.3 | 406 |
| 351 | 103.5 | 174 |
| 352 | 364 | 692 |
| 353 | 38.5 | 140 |
| 354 | 9.6 | 69.7 |
| 355 | 16.3 | 118 |
| 356 | 15.9 | 20.5 |
| 357 | 13.7 | 21.7 |
| 358 | 54.8 | 106 |
| 359 | 35.2 | 109 |
| 360 | 12.9 | 25.2 |
| 361 | 54.6 | 133 |
| 362 | 42.8 | 76.7 |
| 363 | 112 | 152 |
| 364 | 2.5 | 2.6 |
| 365 | 2.5 | 2.5 |
| 366 | 2.6 | 2.5 |
| 367 | 12.24 | 14.1 |
| 368 | 3.531 | 3.1 |
| 369 | <2.5 | <2.5 |
| 370 | 140.4 | 133.3 |
| 371 | 26.95 | 18 |
| 372 | 19.7 | 18.2 |
| 373 | 11.96 | 8.6 |
| 374 | 14.03 | 12.5 |
| 375 | 77.89 | 67.4 |
| 376 | 37.89 | 32.6 |
| 377 | 4.518 | 5.8 |
| 378 | 91.86 | 74.4 |
| 379 | 3.985 | 5.2 |
| 380 | 2.5 | 2.9 |
| 381 | 2.5 | 2.5 |
| 382 | 32.05 | 27.6 |
| 383 | 79.6 | 92.3 |
| 384 | 351.2 | 299 |
| 385 | 124 | 166 |
| 386 | 17.1 | 26 |
| 387 | 15.1 | 35.8 |
| 388 | 59.3 | 132 |
| 389 | 7.8 | 14.1 |
| 390 | 4 | 8.7 |
| 391 | 6.3 | 12.4 |
| 392 | 170.5 | 153 |
| 393 | 48 | 56.9 |
| 394 | 34.2 | 26.1 |
| 395 | 17.3 | 21.2 |
| 396 | 20.5 | 68.1 |
| 397 | 4.4 | 7.7 |
| 398 | 23.2 | 78.6 |
| 399 | 69 | 213 |
| 400 | 214.3 | 736 |
| 401 | 4.4 | 36.2 |
| 402 | 10.1 | 59.5 |
| 403 | 4 | 4.6 |
| 404 | 224.2 | 287.2 |
| 405 | 288.4 | 289.7 |
| 406 | 518.1 | 487.3 |
| 407 | 199.9 | 190.6 |
| 408 | 684.1 | 518.2 |
| 409 | 2.6 | 3.7 |
| 410 | 225.3 | 281.1 |
| 411 | 250.9 | 299.4 |
| 412 | 358.8 | 322.8 |
| 413 | 7397 | 4082 |
| 414 | 6696 | 3586 |
| 415 | 3844 | 3929 |
| 416 | 91.97 | 136.8 |
| 417 | 9.08 | N/A |
| 418 | 2.7 | 2.5 |
| 419 | 345.4 | N/A |
| 420 | 2.85 | 2.7 |
| 421 | 4.874 | 11.9 |
| 422 | 7.842 | 11.7 |
| 423 | 9.699 | 11.5 |
| 424 | 6.693 | 19.7 |
| 425 | 22.8 | 30.8 |
| 426 | 10.25 | 13.7 |
| 427 | 3.629 | 5.4 |
| 428 | 2.5 | 2.5 |
| 429 | 87.18 | 82.1 |
| 430 | 109 | 61.7 |
| 431 | 118.9 | 99.3 |
| 432 | 114.4 | 117.3 |
| 433 | 33.05 | 29.3 |
| 434 | 437.6 | 482.5 |
| 435 | 129.6 | 207.3 |
| 436 | 6.603 | 6.7 |
| 437 | 109 | 152.8 |
| 438 | 21.5 | 27.4 |
| 439 | 2.5 | 2.5 |
| 440 | 2.5 | 2.5 |
| 441 | 2.5 | 2.5 |
| 442 | 5.56 | 5 |
| 443 | 6.171 | 5.1 |
| 444 | <2.5 | <2.5 |
| 445 | 15.5 | 12.7 |
| 446 | 14.74 | 18.2 |
| 447 | 499.7 | 230.7 |
| 448 | 315.9 | 229.6 |
| 449 | 1191 | 811.6 |
| 450 | 226.9 | 174.6 |
| 451 | 14.64 | 16.5 |
| 452 | 63.88 | 71.9 |
| 453 | 9582 | 7864 |
| 454 | 24.46 | 40.3 |
| 455 | 2.693 | 3.6 |
| 456 | 14.1 | 23.2 |
| 457 | 2.5 | 2.5 |
| 458 | 68.1 | 78.6 |
| 459 | 892.5 | 616.4 |
| 460 | 2.5 | 2.5 |
| 461 | 22.51 | 21.1 |
| 462 | 12.12 | 12.5 |
| 463 | 6.7 | 14.8 |
| 464 | 39.7 | 42.6 |

N/A = not available

### Uses of the Substituted Benzothiophene Derivatives

### Treatment or Prevention of Cellular Proliferation Disorders

The present disclosure also relates to methods of treating a cellular proliferative disorder, said methods comprising administering to a subject in need thereof a Substituted Benzothiophene Derivative.

The Substituted Benzothiophene Derivatives disclosed herein are potentially useful in treating diseases or disorders including, but not limited to, cellular proliferative disorders. Cellular proliferation disorders include, but are not limited to, cancers, benign papillomatosis, and gestational trophoblastic diseases. The terms “cancer”, “cancerous”, or “malignant” refer to or describe the physiological condition in mammals that is typically characterized by unregulated cell growth.

In specific embodiments, the cellular proliferative disorder is selected from cancer, benign papillomatosis, benign neoplastic diseases and gestational trophoblastic diseases. In particular embodiments, the gestational trophoblastic disease is selected from the group consisting of hydatidiform moles, and gestational trophoblastic neoplasia (e.g., invasive moles, choriocarcinomas, placental-site trophoblastic tumors, and epithelioid trophoblastic tumors).

In a particular embodiment, the cellular proliferative disorder being treated is cancer.

Accordingly, in one embodiment, provided herein are methods for treating cancer in a patient, the methods comprising administering to the patient an effective amount of a Substituted Benzothiophene Derivative. In a specific embodiment, the amount administered is effective to treat cancer in the patient. In another specific embodiment, the amount administered is effective to inhibit cancer cell replication or cancer cell metastasis in the patient.

In one embodiments, described herein are the use of the Substituted Benzothiophene Derivatives, or a pharmaceutically acceptable salt thereof, for the preparation of a medicament for the treatment of cancer.

In another embodiment, described herein are Substituted Benzothiophene Derivatives, for use in the treatment of cancer.

In one embodiment, the cancer is metastatic. In another embodiment, the cancer is relapsed. In another embodiment, the cancer is refractory. In yet another embodiment, the cancer is relapsed and refractory.

In one embodiment, the patient has previously received treatment for cancer. In another embodiment, the patient has not previously received treatment for cancer.

In one embodiment, the patient has previously received systemic treatment for cancer. In another embodiment, the patient has not previously received systemic treatment for cancer.

In other embodiments, the cancer is present in an adult patient; in additional embodiments, the cancer is present in a pediatric patient.

The compounds, compositions and methods provided herein are useful for the treatment of cancer. Cancers that may be treated using the compounds, compositions and methods disclosed herein include, but are not limited to: (1) Cardiac: sarcoma (angiosarcoma, fibrosarcoma, rhabdomyosarcoma, liposarcoma), myxoma, rhabdomyoma, fibroma, lipoma and teratoma; (2) Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma, non-small cell; (3) Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma), colon, colorectal, rectal; (4) Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); (5) Liver: hepatoma (hepatocellular carcinoma), cholangiocarcinoma, hepatoblastoma, angiosarcoma, hepatocellular adenoma, hemangioma; (6) Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; (7) Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiforme, oligodendroglioma, schwannoma, retinoblastoma, congenital tumors), spinal cord neurofibroma, meningioma, glioma, sarcoma); (8) Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast; (9) Hematologic: blood (myeloid leukemia [acute and chronic], acute lymphoblastic leukemia, chronic lymphocytic leukemia, chronic myelomonocytic (CMML), myelocellular proliferative disorders, multiple myeloma, myelodysplastic syndrome), Hodgkin's disease, non-Hodgkin's lymphoma [malignant lymphoma]; (10) Skin: malignant melanoma, basal cell carcinoma, squamous cell carcinoma, Karposi's sarcoma, moles dysplastic nevi, lipoma, angioma, dermatofibroma, keloids, psoriasis; and (11) Adrenal glands: neuroblastoma. Examples of cancer that may be treated using the compounds, compositions and methods described herein include thyroid cancer, anaplastic thyroid carcinoma, epidermal cancer, head and neck cancer (e.g., squamous cell cancer of the head and neck), sarcoma, tetracarcinoma, hepatoma and multiple myeloma.

The term “cancerous cell” as used herein, includes a cell afflicted by any one of the above-identified conditions.

In particular embodiments, the cancer is selected from brain and spinal cancers, cancers of the head and neck, leukemia and cancers of the blood, skin cancers, cancers of the reproductive system, cancers of the gastrointestinal system, liver and bile duct cancers, kidney and bladder cancers, bone cancers, lung cancers, metastatic microsatellite instability-high (MSI-H) cancer, mismatch repair deficient cancer, malignant mesothelioma, sarcomas, lymphomas, glandular cancers, thyroid cancers, heart tumors, germ cell tumors, malignant neuroendocrine (carcinoid) tumors, midline tract cancers, and cancers of unknown primary origin (i.e., cancers in which a metastasized cancer is found but the original cancer site is not known). In particular embodiments, the cancer is AIDS-related.

In one embodiment, the cancer is bladder cancer. In another embodiment, the cancer is breast cancer. In yet another embodiment, the cancer is NSCLC. In still another embodiment, the cancer is CRC. In another embodiment, the cancer is RCC. In another embodiment, the cancer is HCC. In one embodiment, the cancer is skin cancer. In another embodiment, the skin cancer is melanoma. In another embodiment, the cancer is ovarian cancer. In yet another embodiment, the cancer is pancreatic cancer. In another embodiment, the cancer is a primary or metastatic brain cancer. In still another embodiment, the cancer is CRC.

In one embodiment, provided herein is a method of treating unresectable or metastatic melanoma in a human patient. In some embodiments, the method comprises treating resected high-risk stage III melanoma.

In one embodiment, provided herein is a method of treating metastatic non-small cell lung cancer (NSCLC) in a human patient. In some embodiments, the NSCLC is non-squamous.

In other embodiments, the NSCLC is squamous.

In some embodiments, the cancer exhibits high PD-L1 expression [(Tumor Proportion Score (TPS)≥50%)] and was not previously treated with platinum-containing chemotherapy. In alternative embodiments, the patient has a tumor with PD-L1 expression (TPS≥1%), and was previously treated with platinum-containing chemotherapy. In specific embodiments, the patient had disease progression on or after receiving platinum-containing chemotherapy.

In certain embodiments the PD-L1 TPS is determined by an FDA-approved test.

In certain embodiments of the method for treating NSCLC, the patient's tumor has no EGFR or ALK genomic aberrations.

In certain embodiments of the method for treating NSCLC, the patient's tumor has an EGFR or ALK genomic aberration and had disease progression on or after receiving treatment for the EGFR or ALK aberration(s) prior to receiving combination therapy described herein.

In one embodiment, provided herein is a method of treating recurrent or metastatic head and neck squamous cell cancer (HNSCC) in a human patient. In some embodiments, the patient was previously treated with platinum-containing chemotherapy. In certain embodiments, the patient had disease progression during or after platinum-containing chemotherapy.

In one embodiment, provided herein is a method of treating refractory classical Hodgkin lymphoma (cHL) in a human patient. In certain embodiments, the patient has relapsed after 1, 2, 3 or more lines of therapy for cHL. In specific embodiments, the patient is an adult patient. In alternative embodiments the patient is a pediatric patient.

In one embodiment, provided herein is a method of treating locally advanced or metastatic urothelial carcinoma in a human patient. In certain embodiments, the patient is not eligible for cisplatin-containing chemotherapy. In further embodiments, the patient has disease progression during or following platinum-containing chemotherapy or within 12 months of neoadjuvant or adjuvant treatment with platinum-containing chemotherapy. In specific embodiments, the patient's tumor expresses PD-L1 (CPS ≥10).

In one embodiment, provided herein is a method of treating unresectable or metastatic, microsatellite instability-high (MSI-H) or mismatch repair deficient solid tumors in a human patient. In specific embodiments, the patient had disease progression following prior anti-cancer treatment.

In one embodiment, provided herein is a method of treating unresectable or metastatic, microsatellite instability-high (MSI-H) or mismatch repair deficient colorectal cancer in a human patient. In specific embodiments, the patient had disease progression following prior treatment with a fluoropyrimidine, oxaliplatin, and irinotecan.

In one embodiment, provided herein is a method of treating recurrent locally advanced or metastatic gastric cancer or recurrent locally advanced or metastatic gastroesophageal junction adenocarcinoma in a human patient. In specific embodiments, the patient's tumor expresses PD-L1 [Combined Positive Score (CPS)≥1]. In some embodiments, the patient has disease progression on or after two or more prior lines of therapy including fluoropyrimidine- and platinum-containing chemotherapy. In some embodiments, the patient has disease progression on or after two or more prior lines of therapy including HER2/neu-targeted therapy.

In one embodiment, provided herein is a method of treating non-Hodgkin lymphoma in a human patient. In certain embodiments, the non-Hodgkin lymphoma is primary mediastinal large B-cell lymphoma.

In one embodiment, provided herein is a method of treating breast cancer in a human patient. in specific embodiments, the breast cancer is triple negative breast cancer. In other specific embodiments, the breast cancer is ER+/HER2− breast cancer.

In one embodiment, provided herein is a method of treating cancer in a human patient comprising, wherein the patient has a tumor with a high mutational burden.

In specific embodiments, the cancer is selected from brain and spinal cancers. In particular embodiments, the brain and spinal cancer is selected from the group consisting of anaplastic astrocytomas, glioblastomas, astrocytomas, and estheosioneuroblastomas (also known as olfactory blastomas). In particular embodiments, the brain cancer is selected from the group consisting of astrocytic tumor (e.g., pilocytic astrocytoma, subependymal giant-cell astrocytoma, diffuse astrocytoma, pleomorphic xanthoastrocytoma, anaplastic astrocytoma, astrocytoma, giant cell glioblastoma, glioblastoma, secondary glioblastoma, primary adult glioblastoma, and primary pediatric glioblastoma), oligodendroglial tumor (e.g., oligodendroglioma, and anaplastic oligodendroglioma), oligoastrocytic tumor (e.g., oligoastrocytoma, and anaplastic oligoastrocytoma), ependymoma (e.g., myxopapillary ependymoma, and anaplastic ependymoma); medulloblastoma, primitive neuroectodermal tumor, schwannoma, meningioma, atypical meningioma, anaplastic meningioma, pituitary adenoma, brain stem glioma, cerebellar astrocytoma, cerebral astorcytoma/malignant glioma, visual pathway and hypothalmic glioma, and primary central nervous system lymphoma. In specific instances of these embodiments, the brain cancer is selected from the group consisting of glioma, glioblastoma multiforme, paraganglioma, and suprantentorial primordial neuroectodermal tumors (sPNET). In one embodiment, the brain or spinal cancer is a metastatic brain tumor or tumors.

In specific embodiments, the cancer is selected from cancers of the head and neck, including recurrent or metastatic head and neck squamous cell carcinoma (HNSCC), nasopharyngeal cancers, nasal cavity and paranasal sinus cancers, hypopharyngeal cancers, oral cavity cancers (e.g., squamous cell carcinomas, lymphomas, and sarcomas), lip cancers, oropharyngeal cancers, salivary gland tumors, cancers of the larynx (e.g., laryngeal squamous cell carcinomas, rhabdomyosarcomas), and cancers of the eye or ocular cancers. In particular embodiments, the ocular cancer is selected from the group consisting of intraocular melanoma and retinoblastoma.

In specific embodiments, the cancer is selected from leukemia and cancers of the blood. In particular embodiments, the cancer is selected from the group consisting of myeloproliferative neoplasms, myelodysplastic syndromes, myelodysplastic/myeloproliferative neoplasms, acute myeloid leukemia (AML), myelodysplastic syndrome (MDS), chronic myelogenous leukemia (CML), myeloproliferative neoplasm (MPN), post-MPN AML, post-MDS AML, del(5q)-associated high risk MDS or AML, blast-phase chronic myelogenous leukemia, angioimmunoblastic lymphoma, acute lymphoblastic leukemia, Langerans cell histiocytosis, hairy cell leukemia, and plasma cell neoplasms including plasmacytomas and multiple myelomas. Leukemias referenced herein may be acute or chronic.

In specific embodiments, the cancer is selected from skin cancers. In particular embodiments, the skin cancer is selected from the group consisting of melanoma, squamous cell cancers, and basal cell cancers. In specific embodiments, the skin cancer is unresectable or metastatic melanoma.

In specific embodiments, the cancer is selected from cancers of the reproductive system. In particular embodiments, the cancer is selected from the group consisting of breast cancers, cervical cancers, vaginal cancers, ovarian cancers, endometrial cancers, prostate cancers, penile cancers, and testicular cancers. In specific instances of these embodiments, the cancer is a breast cancer selected from the group consisting of ductal carcinomas and phyllodes tumors. In specific instances of these embodiments, the breast cancer may be male breast cancer or female breast cancer. In some instances of these embodiments, the breast cancer is triple-negative breast cancer. In other instances, the breast cancer is ER+/HER2− breast cancer. In specific instances of these embodiments, the cancer is a cervical cancer selected from the group consisting of squamous cell carcinomas and adenocarcinomas. In specific instances of these embodiments, the cancer is an ovarian cancer selected from the group consisting of epithelial cancers.

In specific embodiments, the cancer is selected from cancers of the gastrointestinal system. In particular embodiments, the cancer is selected from the group consisting of esophageal cancers, gastric cancers (also known as stomach cancers), gastrointestinal carcinoid tumors, pancreatic cancers, gall bladder cancers, colorectal cancers, and anal cancer. In instances of these embodiments, the cancer is selected from the group consisting of esophageal squamous cell carcinomas, esophageal adenocarcinomas, gastric adenocarcinomas, gastrointestinal carcinoid tumors, gastrointestinal stromal tumors, gastric lymphomas, gastrointestinal lymphomas, solid pseudopapillary tumors of the pancreas, pancreatoblastoma, islet cell tumors, pancreatic carcinomas including acinar cell carcinomas and ductal adenocarcinomas, gall bladder adenocarcinomas, colorectal adenocarcinomas, microsatellite stable colorectal cancer, advanced microsatellite stable colorectal cancer, metastatic microsatellite stable colorectal cancer and anal squamous cell carcinomas.

In specific embodiments, the cancer is selected from liver and bile duct cancers. In particular embodiments, the cancer is liver cancer (also known as hepatocellular carcinoma). In particular embodiments, the cancer is bile duct cancer (also known as cholangiocarcinoma); in instances of these embodiments, the bile duct cancer is selected from the group consisting of intrahepatic cholangiocarcinoma and extrahepatic cholangiocarcinoma.

In specific embodiments, the cancer is selected from kidney and bladder cancers. In particular embodiments, the cancer is a kidney cancer selected from the group consisting of renal cell cancer, Wilms tumors, and transitional cell cancers. In particular embodiments, the cancer is a bladder cancer selected from the group consisting of urothelial carcinoma (a transitional cell carcinoma), squamous cell carcinomas, and adenocarcinomas.

In specific embodiments, the cancer is selected from bone cancers. In particular embodiments, the bone cancer is selected from the group consisting of osteosarcoma, malignant fibrous histiocytoma of bone, Ewing sarcoma, chordoma (cancer of the bone along the spine).

In specific embodiments, the cancer is selected from lung cancers. In particular embodiments, the lung cancer is selected from the group consisting of non-small cell lung cancer, small cell lung cancers, bronchial tumors, and pleuropulmonary blastomas.

In specific embodiments, the cancer is selected from malignant mesothelioma. In particular embodiments, the cancer is selected from the group consisting of epithelial mesothelioma and sarcomatoids.

In specific embodiments, the cancer is selected from sarcomas. In particular embodiments, the sarcoma is selected from the group consisting of central chondrosarcoma, central and periosteal chondroma, fibrosarcoma, clear cell sarcoma of tendon sheaths, and Kaposi's sarcoma.

In specific embodiments, the cancer is selected from lymphomas. In particular embodiments, the cancer is selected from the group consisting of Hodgkin lymphoma (e.g., classical Hodgkin refractory lymphoma), non-Hodgkin lymphoma (e.g., diffuse large B-cell lymphoma, follicular lymphoma, mycosis fungoides, Sezary syndrome, primary central nervous system lymphoma), cutaneous T-cell lymphomas, primary central nervous system lymphomas.

In specific embodiments, the cancer is selected from glandular cancers. In particular embodiments, the cancer is selected from the group consisting of adrenocortical cancer (also known as adrenocortical carcinoma or adrenal cortical carcinoma), pheochromocytomas, paragangliomas, pituitary tumors, thymoma, and thymic carcinomas.

In specific embodiments, the cancer is selected from thyroid cancers. In particular embodiments, the thyroid cancer is selected from the group consisting of medullary thyroid carcinomas, papillary thyroid carcinomas, and follicular thyroid carcinomas.

In specific embodiments, the cancer is selected from germ cell tumors. In particular embodiments, the cancer is selected from the group consisting of malignant extracranial germ cell tumors and malignant extragonadal germ cell tumors. In specific instances of these embodiments, the malignant extragonadal germ cell tumors are selected from the group consisting of nonseminomas and seminomas.

In specific embodiments, the cancer is selected from heart tumors. In particular embodiments, the heart tumor is selected from the group consisting of malignant teratoma, lymphoma, rhabdomyosacroma, angiosarcoma, chondrosarcoma, infantile fibrosarcoma, and synovial sarcoma.

In embodiments, the cancer is a metastatic tumor, for example, liver metastases from colorectal cancer or pancreatic cancer; and brain metastases from lung or breast cancer.

In embodiments, the cancer is selected from the group consisting of solid tumors and lymphomas. In particular embodiments, the cancer is selected from the group consisting of advanced or metastatic solid tumors and lymphomas. In more particular embodiments, the cancer is selected from the group consisting of malignant melanoma, head and neck squamous cell carcinoma, breast adenocarcinoma, and lymphomas. In aspects of such embodiments, the lymphomas are selected from the group consisting of diffuse large B-cell lymphoma, follicular lymphoma, mantle cell lymphoma, small lymphocytic lymphoma, mediastinal large B-cell lymphoma, splenic marginal zone B-cell lymphoma, extranodal marginal zone B-cell lymphoma of mucosa-associated lymphoid tissue (malt), nodal marginal zone B-cell lymphoma, lymphoplasmacytic lymphoma, primary effusion lymphoma, Burkitt lymphoma, anaplastic large cell lymphoma (primary cutaneous type), anaplastic large cell lymphoma (systemic type), peripheral T-cell lymphoma, angioimmunoblastic T-cell lymphoma, adult T-cell lymphoma/leukemia, nasal type extranodal NK/T-cell lymphoma, enteropathy-associated T-cell lymphoma, gamma/delta hepatosplenic T-cell lymphoma, subcutaneous panniculitis-like T-cell lymphoma, mycosis fungoides, and Hodgkin lymphoma.

In particular embodiments, the cancer is classified as stage III cancer or stage IV cancer. In some instances of these embodiments, the cancer is not surgically respectable.

### Compositions and Administration

When administered to a patient, a Substituted Benzothiophene Derivative can be administered as a component of a pharmaceutical composition that comprises a pharmaceutically acceptable excipient. Accordingly, in one embodiment, the present disclosure provides pharmaceutical compositions comprising an effective amount of a Substituted Benzothiophene Derivative, and one or more pharmaceutically acceptable carriers or excipients.

The Substituted Benzothiophene Derivatives are useful in preparing a medicament that is useful in treating a cellular proliferative disorder. In one embodiment, the Substituted Benzothiophene Derivatives are also useful for preparing a medicament that is useful in treating cancer.

In the pharmaceutical compositions and methods of the present disclosure, the active ingredients will typically be administered in admixture with suitable carrier materials suitably selected with respect to the intended form of administration, i.e., oral tablets, capsules (either solid-filled, semi-solid filled or liquid filled), powders for constitution, oral gels, elixirs, dispersible granules, syrups, suspensions, and the like, and consistent with conventional pharmaceutical practices. For example, for oral administration in the form of tablets or capsules, the active drug component may be combined with any oral non-toxic pharmaceutically acceptable inert carrier, such as lactose, starch, sucrose, cellulose, magnesium stearate, dicalcium phosphate, calcium sulfate, talc, mannitol, ethyl alcohol (liquid forms), and the like. Solid form preparations include powders, tablets, dispersible granules, capsules, cachets and suppositories. Powders and tablets may be comprised of from about 0.5 to about 95 percent inventive composition. Tablets, powders, cachets and capsules can be used as solid dosage forms suitable for oral administration.

Moreover, when desired or needed, suitable binders, lubricants, disintegrating agents and coloring agents may also be incorporated in the mixture. Suitable binders include starch, gelatin, natural sugars, corn sweeteners, natural and synthetic gums such as acacia, sodium alginate, carboxymethylcellulose, polyethylene glycol and waxes. Suitable lubricants include boric acid, sodium benzoate, sodium acetate, sodium chloride, and the like. Disintegrants include starch, methylcellulose, guar gum, and the like. Sweetening and flavoring agents, and preservatives may also be included where appropriate.

Also included are solid form preparations which are intended to be converted, shortly before use, to liquid form preparations for either oral or parenteral administration. Such liquid forms include solutions, suspensions and emulsions.

For preparing suppositories, a low melting wax such as a mixture of fatty acid glycerides or cocoa butter is first melted, and the active ingredient is dispersed homogeneously therein. The molten homogeneous mixture is then poured into convenient sized molds, allowed to cool, and thereby solidify.

Additionally, the pharmaceutical compositions of the present disclosure may be formulated in sustained release form to provide the rate-controlled release of any one or more of the components or active ingredients to optimize therapeutic effects, i.e., anticancer activity and the like. Suitable dosage forms for sustained release include layered tablets containing layers of varying disintegration rates or controlled release polymeric matrices impregnated with the active components, and shaped in tablet form or capsules containing such impregnated or encapsulated porous polymeric matrices.

In one embodiment, the Substituted Benzothiophene Derivative is administered orally. In another embodiment, the Substituted Benzothiophene Derivative is administered orally in a capsule. In another embodiment, the Substituted Benzothiophene Derivative is administered orally in a tablet.

In another embodiment, the Substituted Benzothiophene Derivative is administered intravenously.

In another embodiment, the Substituted Benzothiophene Derivative is administered via subcutaneous injection.

In another embodiment, the Substituted Benzothiophene Derivative is administered via intertumoral injection.

In another embodiment, the Substituted Benzothiophene Derivative is administered topically. In a specific embodiment, the Substituted Benzothiophene Derivative is formulated as a cream that can be applied topically.

In still another embodiment, the Substituted Benzothiophene Derivative is administered sublingually.

In one embodiment, a pharmaceutical preparation comprising a Substituted Benzothiophene Derivative is in unit dosage form. In such form, the preparation is subdivided into unit doses containing effective amounts of the active components.

Compositions can be prepared using techniques such as conventional mixing, granulating or coating methods; and by using solid dispersion based upon the guidance provided herein. In one embodiment, the present compositions can contain from about 0.10% to about 99% of a Substituted Benzothiophene Derivative by weight or volume. In various embodiments, the present compositions can contain, in one embodiment, from about 1% to about 70%, or from about 5% to about 60%, or from about 10% to about 50% of a Substituted Benzothiophene Derivative by weight or volume.

In one embodiment, the present disclosure provides compositions comprising a Substituted Benzothiophene Derivative, a pharmaceutically acceptable carrier, and one or more additional therapeutic agents. In another embodiment, the present disclosure provides compositions comprising a Substituted Benzothiophene Derivative, a pharmaceutically acceptable carrier, and one additional therapeutic agents. In another embodiment, the present disclosure provides compositions comprising a Substituted Benzothiophene Derivative, a pharmaceutically acceptable carrier, and two additional therapeutic agents.

The quantity of a Substituted Benzothiophene Derivative in a unit dose of preparation may be varied or adjusted from about 1 mg to about 2500 mg. In various embodiments, the quantity is from about 10 mg to about 1000 mg, 1 mg to about 500 mg, 1 mg to about 100 mg, 1 mg to about 50 mg, 1 mg to about 20 mg, and 1 mg to about 10 mg.

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR), e.g., the Physicians' Desk Reference, 71^{st} Edition, 2017 (published by PDR Network, LLC at Montvale, NJ 07645-1725), presently accessible through www.pdr.net; the disclosures of which are incorporated herein by reference thereto.

If the patient is responding, or is stable, after completion of the therapy cycle, the therapy cycle can be repeated according to the judgment of the skilled clinician. Upon completion of multiple therapy cycles, the patient can be continued on the Substituted Benzothiophene Derivatives at the same dose that was administered in the treatment protocol. This maintenance dose can be continued until the patient progresses, or can no longer tolerate the dose (in which case the dose can be reduced and the patient can be continued on the reduced dose).

The doses and dosage regimen of the additional therapeutic agent(s) used in the combination therapies described herein for the treatment of cellular proliferative disorders can be determined by the attending clinician, taking into consideration the approved doses and dosage regimen in the package insert; the age, sex and general health of the patient; and the type and severity of the cellular proliferative disorder. When administered in combination with one or more additional therapeutic agents, the Substituted Benzothiophene Derivative, and the additional therapeutic agent(s) can be administered simultaneously (i.e., in the same composition or in separate compositions one right after the other) or sequentially. This is particularly useful when the components of the combination are given on different dosing schedules, e.g., one component is administered once daily and another component is administered every six hours, or when the preferred pharmaceutical compositions are different, e.g., one is a tablet and one is a capsule. A kit comprising the separate dosage forms can therefore be advantageous.

The attending clinician, in judging whether treatment is effective at the dosage administered, will consider the general well-being of the patient as well as more definite signs such as relief of cancer-related symptoms (e.g., pain), inhibition of tumor growth, actual shrinkage of the tumor, or inhibition of metastasis. Size of the tumor can be measured by standard methods such as radiological studies, e.g., CAT or MRI scan, and successive measurements can be used to judge whether or not growth of the tumor has been retarded or even reversed. Relief of disease-related symptoms such as pain, and improvement in overall condition can also be used to help judge effectiveness of treatment.

Generally, a total daily dosage of a Substituted Benzothiophene Derivative alone, or when administered as combination therapy, can range from about 1 to about 2500 mg per day, although variations will necessarily occur depending on the target of therapy, the patient and the route of administration. In one embodiment, the dosage is from about 10 to about 1000 mg/day, administered in a single dose or in 2-4 divided doses. In another embodiment, the dosage is from about 1 to about 500 mg/day, administered in a single dose or in 2-4 divided doses. In still another embodiment, the dosage is from about 1 to about 100 mg/day, administered in a single dose or in 2-4 divided doses. In yet another embodiment, the dosage is from about 1 to about 50 mg/day, administered in a single dose or in 2-4 divided doses. In another embodiment, the dosage is from about 500 to about 1500 mg/day, administered in a single dose or in 2-4 divided doses. In still another embodiment, the dosage is from about 500 to about 1000 mg/day, administered in a single dose or in 2-4 divided doses. In yet another embodiment, the dosage is from about 100 to about 500 mg/day, administered in a single dose or in 2-4 divided doses.

For convenience, the total daily dosage may be divided and administered in portions during the day if desired. In one embodiment, the daily dosage is administered in one portion. In another embodiment, the total daily dosage is administered in two divided doses over a 24-hour period. In another embodiment, the total daily dosage is administered in three divided doses over a 24-hour period. In still another embodiment, the total daily dosage is administered in four divided doses over a 24-hour period.

The amount and frequency of administration of a Substituted Benzothiophene Derivative will be regulated according to the judgment of the attending clinician considering such factors as age, condition and size of the patient as well as severity of the symptoms being treated.

### Combination Therapy

In one aspect, the present methods for treating a cellular proliferative disorder can further comprise the administration of one or more additional therapeutic agents that are other than a Substituted Benzothiophene Derivative.

Accordingly, in one embodiment, the present disclosure provides methods for treating a cellular proliferative disorder in a patient, the method comprising administering to the patient: (i) a Substituted Benzothiophene Derivative, or a pharmaceutically acceptable salt thereof, and (ii) at least one additional therapeutic agent that is other than a Substituted Benzothiophene Derivative, wherein the amounts administered are together effective to treat a cellular proliferative disorder. In one embodiment, the cellular proliferative disorder treated is cancer.

When administering a combination therapy of the present disclosure to a patient, therapeutic agents in the combination, or a pharmaceutical composition or compositions comprising therapeutic agents, may be administered in any order such as, for example, sequentially, concurrently, together, simultaneously and the like. The amounts of the various actives in such combination therapy may be different amounts (different dosage amounts) or same amounts (same dosage amounts). Thus, for non-limiting illustration purposes, the Substituted Benzothiophene Derivative, and an additional therapeutic agent may be present in fixed amounts (dosage amounts) in a single dosage unit (e.g., a capsule, a tablet and the like).

In one embodiment, the Substituted Benzothiophene Derivative is administered during a time when the additional therapeutic agent(s) exert their prophylactic or therapeutic effect, or vice versa.

In another embodiment, the Substituted Benzothiophene Derivative, and the additional therapeutic agent(s) are administered in doses commonly employed when such agents are used as monotherapy for treating cancer.

In another embodiment, the Substituted Benzothiophene Derivative, and the additional therapeutic agent(s) are administered in doses lower than the doses commonly employed when such agents are used as monotherapy for treating cancer.

In one embodiment, the Substituted Benzothiophene Derivative, and the additional therapeutic agent(s) are present in the same composition. In one embodiment, this composition is suitable for oral administration. In another embodiment, this composition is suitable for intravenous administration. In another embodiment, this composition is suitable for intertumoral administration. In another embodiment, this composition is suitable for subcutaneous administration. In still another embodiment, this composition is suitable for parenteral administration. (none of these types of administration would be preferred for these compounds.)

Cancers and proliferative disorders that can be treated or prevented using the combination therapy methods of the present disclosure include, but are not limited to, those listed above.

The Substituted Benzothiophene Derivative, and the additional therapeutic agent(s) can act additively or synergistically. A synergistic combination may allow the use of lower dosages of one or more agents and/or less frequent administration of one or more agents of a combination therapy. A lower dosage or less frequent administration of one or more agents may lower toxicity of therapy without reducing the efficacy of therapy. Accordingly, in one embodiment, the Substituted Benzothiophene Derivative, and the additional therapeutic agent(s) act synergistically and are administered in doses lower than the doses commonly employed when such agents are used as monotherapy for treating cancer.

In one embodiment, the administration of the Substituted Benzothiophene Derivative, and the additional therapeutic agent(s) may inhibit the resistance of cancer to these agents.

The Substituted Benzothiophene Derivatives may be used in combination with one or more other active agents (collectively referred to herein as “additional therapeutic agents”), including but not limited to, other therapeutic agents that are used in the prevention, treatment, control, amelioration, or reduction of risk of a particular disease or condition (e.g., cancer). In one embodiment, a Substituted Benzothiophene Derivative is combined with one or more other therapeutic agents for use in the prevention, treatment, control amelioration, or reduction of risk of a particular disease or condition for which the Substituted Benzothiophene Derivatives are useful. Such other active agents may be administered, by a route and in an amount commonly used therefor, contemporaneously or sequentially with a compound of the present disclosure.

Combinations of the Substituted Benzothiophene Derivatives with one or more anticancer agents are within the scope of the present disclosure. Examples of such additional anticancer agents can be found in Cancer Principles and Practice of Oncology by V. T. Devita and S. Hellman (editors), 9^{th} edition (May 16, 2011), Lippincott Williams & Wilkins Publishers. A person of ordinary skill in the art would be able to discern which combinations of additional therapeutic agents would be useful based on the particular characteristics of the drugs and the cancer involved. Such additional therapeutic agents include the following: estrogen receptor modulators, programmed cell death protein 1 (PD-1) inhibitors, programmed death-ligand 1 (PD-L1) inhibitors, androgen receptor modulators, retinoid receptor modulators, cytotoxic/cytostatic agents, antiproliferative agents, prenyl-protein transferase inhibitors, HMG-CoA reductase inhibitors and other angiogenesis inhibitors, HIV protease inhibitors, reverse transcriptase inhibitors, inhibitors of cell proliferation and survival signaling, bisphosphonates, aromatase inhibitors, siRNA therapeutics, γ-secretase inhibitors, agents that interfere with receptor tyrosine kinases (RTKs), and agents that interfere with cell cycle checkpoints.

The additional therapeutic agents, and classes of additional therapeutic agents, disclosed below herein, are all useful in the combination therapies described herein.

“Androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

“Estrogen receptor modulators” refers to compounds that interfere with or inhibit the binding of estrogen to the receptor, regardless of mechanism. Examples of estrogen receptor modulators include, but are not limited to, tamoxifen, raloxifene, idoxifene, LY353381, LY117081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]-phenyl-2,2-dimethylpropanoate, 4,4′-dihydroxybenzophenone-2,4-dinitrophenyl-hydrazone, and SH646.

In the treatment of breast cancer (e.g., postmenopausal and premenopausal breast cancer, e.g., hormone-dependent breast cancer) the compound of formula (1) may be used with an effective amount of at least one antihormonal agent selected from the group consisting of: (a) aromatase inhibitors, (b) antiestrogens, and (c) LHRH analogues; and optionally an effective amount of at least one chemotherapeutic agent. Examples of aromatase inhibitors include but are not limited to: Anastrozole (e.g., Arimidex), Letrozole (e.g., Femara), Exemestane (Aromasin), Fadrozole and Formestane (e.g., Lentaron). Examples of antiestrogens include but are not limited to: Tamoxifen (e.g., Nolvadex), Fulvestrant (e.g., Faslodex), Raloxifene (e.g., Evista), and Acolbifene.

Examples of LHRH analogues include but are not limited to: goserelin (e.g., Zoladex), and leuprolide (e.g., leuprolide acetate, such as Lupron or Lupron Depot). Examples of additional therapeutic agents useful in the present compositions and methods include, but are not limited to, the following cancer chemotherapeutic agents: trastuzumab (e.g., Herceptin), gefitinib (e.g., Iressa), erlotinib (e.g., erlotinib HCl, such as Tarceva), bevacizumab (e.g., Avastin), cetuximab (e.g., Erbitux), and bortezomib (e.g., Velcade).

“Retinoid receptor modulators” refers to compounds which interfere or inhibit the binding of retinoids to the receptor, regardless of mechanism. Examples of such retinoid receptor modulators include bexarotene, tretinoin, 13-cis-retinoic acid, 9-cis-retinoic acid, a-difluoromethylornithine, ILX23-7553, trans-N-(4′-hydroxyphenyl) retinamide, and N-4-carboxyphenyl retinamide.

“Cytotoxic/cytostatic agents” refers to compounds which cause cell death or inhibit cell proliferation primarily by interfering directly with the cell's functioning or inhibit or interfere with cell mycosis, including alkylating agents, tumor necrosis factors, intercalators, hypoxia activatable compounds, microtubule inhibitors/microtubule-stabilizing agents, inhibitors of mitotic kinesins, histone deacetylase inhibitors, inhibitors of kinases involved in mitotic progression, inhibitors of kinases involved in growth factor and cytokine signal transduction pathways, antimetabolites, biological response modifiers, hormonal/anti-hormonal therapeutic agents, haematopoietic growth factors, monoclonal antibody targeted therapeutic agents, topoisomerase inhibitors, proteosome inhibitors, ubiquitin ligase inhibitors, and aurora kinase inhibitors.

Examples of cytotoxic/cytostatic agents include, but are not limited to, sertenef, cachectin, ifosfamide, tasonermin, lonidamine, carboplatin, altretamine, prednimustine, dibromodulcitol, ranimustine, fotemustine, nedaplatin, oxaliplatin, temozolomide, heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichloro(2-methyl-pyridine)platinum, benzylguanine, glufosfamide, GPX100, (trans, trans, trans)-bis-mu-(hexane-1,6-diamine)-mu-[diamine-platinum(II)]bis[diamine(chloro)platinum (II)]tetrachloride, diarizidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deamino-3′-morpholino-13-deoxo-10-hydroxycarminomycin, annamycin, galarubicin, elinafide, MEN10755, 4-demethoxy-3-deamino-3-aziridinyl-4-methylsulphonyl-daunorubicin (see WO 00/50032), Raf kinase inhibitors (such as Bay43-9006), and mTOR inhibitors (such as Wyeth's CCI-779).

An example of a hypoxia activatable compound is tirapazamine.

Examples of proteosome inhibitors include but are not limited to lactacystin and MLN-341 (Velcade).

Examples of microtubule inhibitors/microtubule-stabilizing agents include paclitaxel, vindesine sulfate, 3′,4′-didehydro-4′-deoxy-8′-norvincaleukoblastine, docetaxol, rhizoxin, dolastatin, mivobulin isethionate, auristatin, cemadotin, RPR109881, BMS184476, vinflunine, cryptophycin, 2,3,4,5,6-pentafluoro-N-(3-fluoro-4-methoxyphenyl) benzene sulfonamide, anhydrovinblastine, TDX258, the epothilones (see for example U.S. Pat. Nos. 6,284,781 and 6,288,237), and BMS188797. In an example the epothilones are not included in the microtubule inhibitors/microtubule-stabilising agents.

Some examples of topoisomerase inhibitors are topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-kl]acridine-2-(6H) propanamine, 1-amino-9-ethyl-5-fluoro-2,3-dihydro-9-hydroxy-4-methyl-1H,12H-benzo[de]pyrano[3′,4′:b,7]-indolizino[1,2b]quinoline-10,13(9H,15H)dione, lurtotecan, 7-[2-(N-isopropylamino)ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a, 5aB, 8aa,9b)-9-[2-[N-[2-(dimethylamino)ethyl]-N-methylamino]ethyl]-5-[4-hydroxy-3,5-dimethoxyphenyl]-5,5a,6,8,8a,9-hexohydrofuro(3′,4′:6,7)naphtho(2,3-d)-1,3-dioxol-6-one, 2,3-(methylenedioxy)-5-methyl-7-hydroxy-8-methoxybenzo[c]-phenanthridinium, 6,9-bis[(2-aminoethyl)amino]benzo[g]isoguinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2,1-c]quinolin-7-one, and dimesna.

Examples of inhibitors of mitotic kinesins, and in particular the human mitotic kinesin KSP, are described in Publications WO03/039460, WO03/050064, WO03/050122, WO03/049527, WO03/049679, WO03/049678, WO04/039774, WO03/079973, WO03/099211, WO03/105855, WO03/106417, WO04/037171, WO04/058148, WO04/058700, WO04/126699, WO05/018638, WO05/019206, WO05/019205, WO05/018547, WO05/017190, US2005/0176776. In an example inhibitors of mitotic kinesins include, but are not limited to inhibitors of KSP, inhibitors of MKLP1, inhibitors of CENP-E, inhibitors of MCAK and inhibitors of Rab6-KIFL.

Examples of “histone deacetylase inhibitors” include, but are not limited to, SAHA, TSA, oxamflatin, PXD101, MG98 and scriptaid. Further reference to other histone deacetylase inhibitors may be found in the following manuscript; Miller, T. A. et al. J. Med. Chem. 46(24):5097-5116 (2003).

“Inhibitors of kinases involved in mitotic progression” include, but are not limited to, inhibitors of aurora kinase, inhibitors of Polo-like kinases (PLK; in particular inhibitors of PLK-1), inhibitors of bub-1 and inhibitors of bub-R1. An example of an “aurora kinase inhibitor” is VX-680 (tozasertib).

“Antiproliferative agents” include antisense RNA and DNA oligonucleotides such as G3139, ODN698, GEM231, and INX3001, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxycytidine, N-[5-(2,3-dihydro-benzofuryl)sulfonyl]-N′-(3,4-dichlorophenyl)urea, N6-[4-deoxy-4-[N2-[2(E),4(E)-tetradecadienoyl]glycylamino]-L-glycero-B-L-manno-heptopyranosyl]adenine, aplidine, ecteinascidin, troxacitabine, 4-[2-amino-4-oxo-4,6,7,8-tetrahydro-3H-pyrimidino[5,4-b][1,4]thiazin-6-yl-(S)-ethyl]-2,5-thienoyl-L-glutamic acid, aminopterin, 5-flurouracil, alanosine, 11-acetyl-8-(carbamoyloxymethyl)-4-formyl-6-methoxy-14-oxa-1,11-diazatetracyclo(7.4.1.0.0)-tetradeca-2,4,6-trien-9-yl acetic acid ester, swainsonine, lometrexol, dexrazoxane, methioninase, 2′-cyano-2′-deoxy-N4-palmitoyl-1-B-D-arabino furanosyl cytosine, 3-aminopyridine-2-carboxaldehyde thiosemicarbazone and trastuzumab.

Examples of monoclonal antibody targeted therapeutic agents include those therapeutic agents which have cytotoxic agents or radioisotopes attached to a cancer cell specific or target cell specific monoclonal antibody. In one embodiment, a monoclonal antibody targeted therapeutic agent is Bexxar.

“HMG-CoA reductase inhibitor” refers to inhibitors of 3-hydroxy-3-methylglutaryl-CoA reductase. Examples of HMG-CoA reductase inhibitors that may be used include but are not limited to lovastatin, simvastatin, pravastatin, fluvastatin, atorvastatin, rosuvastatin and cerivastatin. The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and therefore the use of such salts, esters, open-acid and lactone forms is included within the scope of the present disclosure.

“Prenyl-protein transferase inhibitor” refers to a compound which inhibits any one or any combination of the prenyl-protein transferase enzymes, including farnesyl-protein transferase (FPTase), geranylgeranyl-protein transferase type I (GGPTase-I), and geranylgeranyl-protein transferase type-II (GGPTase-II, also called Rab GGPTase). For an example of the role of a prenyl-protein transferase inhibitor on angiogenesis see European J. of Cancer, Vol. 35, No. 9, pp. 1394-1401 (1999).

“Angiogenesis inhibitor” refers to compounds that inhibit the formation of new blood vessels, regardless of mechanism. Examples of angiogenesis inhibitors include, but are not limited to, tyrosine kinase inhibitors, such as inhibitors of the tyrosine kinase receptors Flt-1 (VEGFR1), and Flk-1/KDR (VEGFR2), inhibitors of epidermal-derived, fibroblast-derived, or platelet derived growth factors, MMP (matrix metalloprotease) inhibitors, integrin blockers, interferon-a, interleukin-12, pentosan polysulfate, cyclooxygenase inhibitors, including nonsteroidal anti-inflammatories (NSAIDs) like aspirin and ibuprofen as well as selective cyclooxy-genase-2 inhibitors like celecoxib and rofecoxib, steroidal anti-inflammatories (such as corticosteroids, mineralocorticoids, dexamethasone, prednisone, prednisolone, methylpred, betamethasone), carboxyamidotriazole, combretastatin A-4, squalamine, 6-O-chloroacetyl-carbonyl)-fumagillol, thalidomide, angiostatin, troponin-1, angiotensin II antagonists.

Other examples of angiogenesis inhibitors useful in the present combinations include, but are not limited to, endostatin, ukrain, ranpirnase, IM862, 5-methoxy-4-[2-methyl-3-(3-methyl-2-butenyl)oxiranyl]-1-oxaspiro[2,5]oct-6-yl(chloroacetyl)carbamate, acetyldinanaline, 5-amino-1-[[3,5-dichloro-4-(4-chlorobenzoyl)phenyl]methyl]-1H-1,2,3-triazole-4-carboxamide, CM101, squalamine, combretastatin, RPI4610, NX31838, sulfated mannopentaose phosphate, 7,7-(carbonyl-bis[imino-N-methyl-4,2-pyrrolocarbonylimino[N-methyl-4,2-pyrrole]-carbonylimino]-bis-(1,3-naphthalene disulfonate), and 3-[(2,4-dimethylpyrrol-5-yl)methylene]-2-indolinone (SU5416), or a pharmaceutically acceptable salt thereof.

Additional therapeutic agents that modulate or inhibit angiogenesis and may also be used in combination with the Substituted Benzothiophene Derivatives, include agents that modulate or inhibit the coagulation and fibrinolysis systems (see review in Clin. Chem. La. Med. 38:679-692 (2000)). Examples of such agents include, but are not limited to, heparin, low molecular weight heparins and carboxypeptidase U inhibitors (also known as inhibitors of active thrombin activatable fibrinolysis inhibitor [TAFIa]).

Further examples of angiogenesis inhibitors include a tyrosine kinase inhibitor, an inhibitor of epidermal-derived growth factor, an inhibitor of fibroblast-derived growth factor, an inhibitor of platelet derived growth factor, an MMP (matrix metalloprotease) inhibitor, an integrin blocker, interferon-a, interleukin-12, pentosan polysulfate, a cyclooxygenase inhibitor, carboxyamidotriazole, combretastatin A-4, squalamine, 6-O-chloroacetyl-carbonyl)-fumagillol, thalidomide, angiostatin, troponin-1, or an antibody to VEGF.

“Agents that interfere with cell cycle checkpoints” refers to compounds that inhibit protein kinases that transduce cell cycle checkpoint signals, thereby sensitizing the cancer cell to DNA damaging agents. Such agents include inhibitors of ATR, ATM, the CHK1 and CHK2 kinases and cdk and cdc kinase inhibitors and are specifically exemplified by 7-hydroxystaurosporin, flavopiridol, CYC202 (Cyclacel), and BMS-387032.

“Agents that interfere with receptor tyrosine kinases (RTKs)” refers to compounds that inhibit RTKs and therefore mechanisms involved in oncogenesis and tumor progression. Such agents include inhibitors of c-Kit, Eph, PDGF, Flt3 and c-Met. Further agents include inhibitors of RTKs as described by Bume-Jensen and Hunter, Nature, 411:355-365, 2001. Specific examples of tyrosine kinase inhibitors include N-(trifluoromethylphenyl)-5-methylisoxazol-4-carboxamide, 3-[(2,4-dimethylpyrrol-5-yl)methylidenyl)indolin-2-one, 17-(allylamino)-17-demethoxygeldanamycin, 4-(3-chloro-4-fluorophenylamino)-7-methoxy-6-[3-(4-morpholinyl)propoxyl]quinazoline, N-(3-ethynylphenyl)-6,7-bis(2-methoxyethoxy)-4-quinazolinamine, BIBX1382, 2,3,9,10,11,12-hexahydro-10-(hydroxymethyl)-10-hydroxy-9-methyl-9,12-epoxy-TH-diindolo[1,2,3-fg:3′,2′,1′-kl]pyrrolo[3,4-i][1,6]benzodiazocin-1-one, SH268, genistein, STI571, CEP2563, 4-(3-chlorophenylamino)-5,6-dimethyl-7H-pyrrolo[2,3-d]pyrimidinemethane sulfonate, 4-(3-bromo-4-hydroxyphenyl)amino-6,7-dimethoxyquinazoline, 4-(4′-hydroxyphenyl)amino-6,7-dimethoxyquinazoline, SU6668, STI571A, N-4-chlorophenyl-4-(4-pyridylmethyl)-1-phthalazinamine, and EMD121974, or a pharmaceutically acceptable salt thereof.

“Inhibitors of cell proliferation and survival signaling pathway” refers to compounds that inhibit signal transduction cascades downstream of cell surface receptors. Such agents include inhibitors of serine/threonine kinases (including but not limited to inhibitors of Akt such as described in WO 02/083064, WO 02/083139, WO 02/083140, US 2004-0116432, WO 02/083138, US 2004/0102360, WO 03/086404, WO 03/086279, WO 03/086394, WO 03/084473, WO 03/086403, WO 2004/041162, WO 2004/096131, WO 2004/096129, WO 2004/096135, WO 2004/096130, WO 2005/100356, WO 2005/100344, U.S. Pat. Nos. 7,454,431, 7,589,068), inhibitors of Raf kinase (for example BAY-43-9006), inhibitors of MEK (for example CI-1040 and PD-098059), inhibitors of mTOR (for example Wyeth CCI-779), and inhibitors of PI3K (for example LY294002).

The present disclosure also encompasses combination therapies comprising NSAIDs which are selective COX-2 inhibitors. For purposes of the specification NSAIDs which are selective inhibitors of COX-2 are defined as those which possess a specificity for inhibiting COX-2 over COX-1 of at least 100-fold as measured by the ratio of IC₅₀ for COX-2 over IC₅₀ for COX-1 evaluated by cell or microsomal assays. Inhibitors of COX-2 that are useful in the present methods are: 3-phenyl-4-(4-(methylsulfonyl)phenyl)-2-(5H)-furanone; and 5-chloro-3-(4-methylsulfonyl)-phenyl-2-(2-methyl-5-pyridinyl)pyridine; or a pharmaceutically acceptable salt thereof. Compounds that have been described as specific inhibitors of COX-2 and are therefore also useful in the present disclosure include, but are not limited to, the following: rofecoxib, etoricoxib, parecoxib, BEXTRA® and CELEBREX® or a pharmaceutically acceptable salt thereof.

As used herein, “integrin blockers” refers to compounds which selectively antagonize, inhibit or counteract binding of a physiological ligand to the α_{V}β₃ integrin, to compounds which selectively antagonize, inhibit or counteract binding of a physiological ligand to the α_{V}β₅ integrin, to compounds which antagonize, inhibit or counteract binding of a physiological ligand to both the α_{V}β₃ integrin and the α_{V}β₅ integrin, and to compounds which antagonize, inhibit or counteract the activity of the particular integrin(s) expressed on capillary endothelial cells. The term also refers to antagonists of the α_{V}β₆, α_{V}β₈, α₁β₁, α₂β₁, α₅β₁, α₆β₁ and α₆β₄ integrins. The term also refers to antagonists of any combination of α_{V}β₃, α_{V}β₅, α_{V}β₆, α_{V}β₈, α₁β₁, α₂β₁, α₅β₁, α₆β₁ and α₆β₄ integrins.

Combinations with additional therapeutic agents, other than anti-cancer agents, are also contemplated in the instant methods. For example, combinations of the Substituted Benzothiophene Derivatives with PPAR-γ (i.e., PPAR-gamma) agonists and PPAR-δ (i.e., PPAR-delta) agonists are useful in the treatment of certain malignancies. PPAR-γ and PPAR-δ are the nuclear peroxisome proliferator-activated receptors γ and δ. PPAR-γ agonists have been shown to inhibit the angiogenic response to VEGF in vitro; both troglitazone and rosiglitazone maleate inhibit the development of retinal neovascularization in mice (Arch. Ophthamol. 2001; 119:709-717). Examples of PPAR-γ agonists and PPAR-γ/α agonists include, but are not limited to, thiazolidinediones (such as DRF2725, CS-011, troglitazone, rosiglitazone, and pioglitazone), fenofibrate, gemfibrozil, clofibrate, GW2570, SB219994, AR-H039242, JTT-501, MCC-555, GW2331, GW409544, NN2344, KRP297, NP0110, DRF4158, NN622, G1262570, PNU182716, DRF552926, 2-[(5,7-dipropyl-3-trifluoromethyl-1,2-benzisoxazol-6-yl)oxy]-2-methylpropionic acid (disclosed in U.S. Ser. No. 09/782,856), and 2(R)-7-(3-(2-chloro-4-(4-fluorophenoxy) phenoxy)propoxy)-2-ethylchromane-2-carboxylic acid (disclosed in U.S. Ser. No. 60/235,708 and 60/244,697), or a pharmaceutically acceptable salt thereof.

Another embodiment of the present disclosure is the use of the Substituted Benzothiophene Derivatives in combination with gene therapy for the treatment of cancer. For an overview of genetic strategies to treating cancer see Hall et al., (Am. J. Hum. Genet. 61:785-789, 1997), and Kufe et al., (Cancer Medicine, 5th Ed, pp 876-889, BC Decker, Hamilton 2000). Gene therapy can be used to deliver any tumor suppressing gene. Examples of such genes include, but are not limited to, p53, which can be delivered via recombinant virus-mediated gene transfer (see U.S. Pat. No. 6,069,134, for example), a uPA/uPAR antagonist (“Adenovirus-Mediated Delivery of a uPA/uPAR Antagonist Suppresses Angiogenesis-Dependent Tumor Growth and Dissemination in Mice,” Gene Therapy, August 1998; 5(8):1105-13), and interferon gamma (J. Immunol. 2000; 164:217-222).

The Substituted Benzothiophene Derivatives may also be administered in combination with an inhibitor of inherent multidrug resistance (MDR), in particular MDR associated with high levels of expression of transporter proteins. Such MDR inhibitors include inhibitors of p-glycoprotein (P-gp), such as LY335979, XR9576, OC144-093, R101922, VX853 and PSC833 (valspodar), or a pharmaceutically acceptable salt thereof.

A Substituted Benzothiophene Derivative may also be administered with an immunologic-enhancing drug, such as levamisole, isoprinosine and Zadaxin, or a pharmaceutically acceptable salt thereof.

A Substituted Benzothiophene Derivative may also be useful for treating or preventing cancer in combination with P450 inhibitors including: xenobiotics, quinidine, tyramine, ketoconazole, testosterone, quinine, methyrapone, caffeine, phenelzine, doxorubicin, troleandomycin, cyclobenzaprine, erythromycin, cocaine, furafyline, cimetidine, dextromethorphan, ritonavir, indinavir, amprenavir, diltiazem, terfenadine, verapamil, cortisol, itraconazole, mibefradil, nefazodone and nelfinavir, or a pharmaceutically acceptable salt thereof.

A Substituted Benzothiophene Derivative may also be useful for treating or preventing cancer in combination with Pgp and/or BCRP inhibitors including: cyclosporin A, PSC833, GF120918, cremophorEL, fumitremorgin C, Ko132, Ko134, Iressa, Imatnib mesylate, EKI-785, C11033, novobiocin, diethylstilbestrol, tamoxifen, resperpine, VX-710, tryprostatin A, flavonoids, ritonavir, saquinavir, nelfinavir, omeprazole, quinidine, verapamil, terfenadine, ketoconazole, nifidepine, FK506, amiodarone, XR9576, indinavir, amprenavir, cortisol, testosterone, LY335979, OC144-093, erythromycin, vincristine, digoxin and talinolol, or a pharmaceutically acceptable salt thereof.

A Substituted Benzothiophene Derivative may also be useful for treating or preventing cancer, including bone cancer, in combination with bisphosphonates, including but not limited to: etidronate (Didronel), pamidronate (Aredia), alendronate (Fosamax), risedronate (Actonel), zoledronate (Zometa), ibandronate (Boniva), incadronate or cimadronate, clodronate, EB-1053, minodronate, neridronate, piridronate and tiludronate including any and all pharmaceutically acceptable salts, derivatives, hydrates and mixtures thereof.

A Substituted Benzothiophene Derivative may also be useful for treating or preventing breast cancer in combination with aromatase inhibitors. Examples of aromatase inhibitors include but are not limited to: anastrozole, letrozole and exemestane, or a pharmaceutically acceptable salt thereof.

A Substituted Benzothiophene Derivative may also be useful for treating or preventing cancer in combination with siRNA therapeutics.

The Substituted Benzothiophene Derivatives may also be administered in combination with γ-secretase inhibitors and/or inhibitors of NOTCH signaling. Such inhibitors include compounds described in WO 01/90084, WO 02/30912, WO 01/70677, WO 03/013506, WO 02/36555, WO 03/093252, WO 03/093264, WO 03/093251, WO 03/093253, WO 2004/039800, WO 2004/039370, WO 2005/030731, WO 2005/014553, U.S. Ser. No. 10/957,251, WO 2004/089911, WO 02/081435, WO 02/081433, WO 03/018543, WO 2004/031137, WO 2004/031139, WO 2004/031138, WO 2004/101538, WO 2004/101539 and WO 02/47671 (including LY-450139), or a pharmaceutically acceptable salt thereof.

In one embodiment, specific anticancer agents useful in the present combination therapies include, but are not limited to: pembrolizumab (Keytruda®), abarelix (Plenaxis Depot®); aldesleukin (Prokine®); Aldesleukin (Proleukin®); Alemtuzumabb (Campath®); alitretinoin (Panretin®); allopurinol (Zyloprim®); altretamine (Hexalen®); amifostine (Ethyol®); anastrozole (Arimidex®); arsenic trioxide (Trisenox®); asparaginase (Elspar®); azacitidine (Vidaza®); bevacuzimab (Avastin®); bexarotene capsules (Targretin®); bexarotene gel (Targretin®); bleomycin (Blenoxane®); bortezomib (Velcade®); busulfan intravenous (Busulfex®); busulfan oral (Myleran®); calusterone (Methosarb®); capecitabine (Xeloda®); carboplatin (Paraplatin®); carmustine (BCNU®, BiCNU®); carmustine (Gliadel®); carmustine with Polifeprosan 20 Implant (Gliadel Wafer®); celecoxib (Celebrex®); cetuximab (Erbitux®); chlorambucil (Leukeran®); cisplatin (Platinol®); cladribine (Leustatin®, 2-CdA®); clofarabine (Clolar®); cyclophosphamide (Cytoxan®, Neosar®); cyclophosphamide (Cytoxan Injection®); cyclophosphamide (Cytoxan Tablet®); cytarabine (Cytosar-U®); cytarabine liposomal (DepoCyt®); dacarbazine (DTIC-Dome®); dactinomycin, actinomycin D (Cosmegen®); Darbepoetin alfa (Aranesp®); daunorubicin liposomal (DanuoXome®); daunorubicin, daunomycin (Daunorubicin®); daunorubicin, daunomycin (Cerubidine®); Denileukin diftitox (Ontak®); dexrazoxane (Zinecard®); docetaxel (Taxotere®); doxorubicin (Adriamycin PFS®); doxorubicin (Adriamycin®, Rubex®); doxorubicin (Adriamycin PFS Injection®); doxorubicin liposomal (Doxil®); dromostanolone propionate (Dromostanolone®); dromostanolone propionate (Masterone Injection®); Elliott's B Solution (Elliott's B Solution®); epirubicin (Ellence®); Epoetin alfa (Epogen®); erlotinib (Tarceva®); estramustine (Emcyt®); etoposide phosphate (Etopophos®); etoposide, VP-16 (Vepesid®); exemestane (Aromasin®); Filgrastim (Neupogen®); floxuridine (intraarterial) (FUDR®); fludarabine (Fludara®); fluorouracil, 5-FU (Adrucil®); fulvestrant (Faslodex®); gefitinib (Iressa®); gemcitabine (Gemzar®); gemtuzumab ozogamicin (Mylotarg®); goserelin acetate (Zoladex Implant®); goserelin acetate (Zoladex®); histrelin acetate (Histrelin Implant®); hydroxyurea (Hydrea®); Ibritumomab Tiuxetan (Zevalin®); idarubicin (Idamycin®); ifosfamide (IFEX®); imatinib mesylate (Gleevec®); interferon alfa 2a (Roferon A®); Interferon alfa-2b (Intron A®); irinotecan (Camptosar®); lenalidomide (Revlimid®); letrozole (Femara®); leucovorin (Wellcovorin®, Leucovorin®); Leuprolide Acetate (Eligard®); levamisole (Ergamisol®); lomustine, CCNU (CeeBU®); meclorethamine, nitrogen mustard (Mustargen®); megestrol acetate (Megace®); melphalan, L-PAM (Alkeran®); mercaptopurine, 6-MP (Purinethol®); mesna (Mesnex®); mesna (Mesnex Tabs®); methotrexate (Methotrexate®); methoxsalen (Uvadex®); mitomycin C (Mutamycin®); mitotane (Lysodren®); mitoxantrone (Novantrone®); nandrolone phenpropionate (Durabolin-50®); nelarabine (Arranon®); Nofetumomab (Verluma®); Oprelvekin (Neumega®); oxaliplatin (Eloxatin®); paclitaxel (Paxene®); paclitaxel (Taxol®); paclitaxel protein-bound particles (Abraxane®); palifermin (Kepivance®); pamidronate (Aredia®); pegademase (Adagen (Pegademase Bovine)®); pegaspargase (Oncaspar®); Pegfilgrastim (Neulasta®); pemetrexed disodium (Alimta®); pentostatin (Nipent®); pipobroman (Vercyte®); plicamycin, mithramycin (Mithracin®); porfimer sodium (Photofrin®); procarbazine (Matulane®); quinacrine (Atabrine®); Rasburicase (Elitek®); Rituximab (Rituxan®); Ridaforolimus; sargramostim (Leukine®); Sargramostim (Prokine®); sorafenib (Nexavar®); streptozocin (Zanosar®); sunitinib maleate (Sutent®); talc (Sclerosol®); tamoxifen (Nolvadex®); temozolomide (Temodar®); teniposide, VM-26 (Vumon®); testolactone (Teslac®); thioguanine, 6-TG (Thioguanine®); thiotepa (Thioplex®); topotecan (Hycamtin®); toremifene (Fareston®); Tositumomab (Bexxar®); Tositumomab/I-131 tositumomab (Bexxar®); Trastuzumab (Herceptin®); tretinoin, ATRA (Vesanoid®); Uracil Mustard (Uracil Mustard Capsules®); valrubicin (Valstar®); vinblastine (Velban®); vincristine (Oncovin®); vinorelbine (Navelbine®); vorinostat (Zolinza®), and zoledronate (Zometa®), or a pharmaceutically acceptable salt thereof.

Thus, the scope of the present disclosure encompasses the use of the Substituted Benzothiophene Derivatives in combination with a second compound selected from: an estrogen receptor modulator, an androgen receptor modulator, a retinoid receptor modulator, a cytotoxic/cytostatic agent, an antiproliferative agent, a prenyl-protein transferase inhibitor, an HMG-CoA reductase inhibitor, an HIV protease inhibitor, a reverse transcriptase inhibitor, an angiogenesis inhibitor, PPAR-γ agonists, PPAR-δ agonists, an inhibitor of inherent multidrug resistance, an anti-emetic agent, an agent useful in the treatment of anemia, an agent useful in the treatment of neutropenia, an immunologic-enhancing drug, an inhibitor of cell proliferation and survival signaling, a bisphosphonate, an aromatase inhibitor, an siRNA therapeutic, γ-secretase and/or NOTCH inhibitors, agents that interfere with receptor tyrosine kinases (RTKs), an agent that interferes with a cell cycle checkpoint, and any of the therapeutic agents listed above.

Yet another example of the present disclosure is a method of treating cancer that comprises administering a therapeutically effective amount of a Substituted Benzothiophene Derivative in combination with paclitaxel or trastuzumab.

The therapeutic combination disclosed herein may be used in combination with one or more other active agents, including but not limited to, other anti-cancer agents that are used in the prevention, treatment, control, amelioration, or reduction of risk of a particular disease or condition (e.g., cell-proliferation disorders). In one embodiment, a Substituted Benzothiophene Derivative is combined with one or more other anti-cancer agents for use in the prevention, treatment, control amelioration, or reduction of risk of a particular disease or condition for which the Substituted Benzothiophene Derivatives are useful. Such other active agents may be administered, by a route and in an amount commonly used therefor, prior to, contemporaneously, or sequentially with a compound of the present disclosure.

The present disclosure also includes a pharmaceutical composition useful for treating or preventing cancer that comprises a therapeutically effective amount of a Substituted Benzothiophene Derivative and a second compound selected from: an estrogen receptor modulator, an androgen receptor modulator, a retinoid receptor modulator, a cytotoxic/cytostatic agent, an antiproliferative agent, a prenyl-protein transferase inhibitor, an HMG-CoA reductase inhibitor, an HIV protease inhibitor, a reverse transcriptase inhibitor, an angiogenesis inhibitor, a PPAR-γ agonist, a PPAR-δ agonist, an inhibitor of cell proliferation and survival signaling, a bisphosphonate, an aromatase inhibitor, an siRNA therapeutic, γ-secretase and/or NOTCH inhibitors, agents that interfere with receptor tyrosine kinases (RTKs), an agent that interferes with a cell cycle checkpoint, and any of the therapeutic agents listed above.

The present disclosure further relates to a method of treating cancer in a human patient comprising administration of a Substituted Benzothiophene Derivative and a PD-1 antagonist to the patient. The compound of the present disclosure and the PD-1 antagonist may be administered concurrently or sequentially.

In particular embodiments, the PD-1 antagonist is an anti-PD-1 antibody, or antigen binding fragment thereof. In alternative embodiments, the PD-1 antagonist is an anti-PD-L1 antibody, or antigen binding fragment thereof. In some embodiments, the PD-1 antagonist is an anti-PD-1 antibody, independently selected from pembrolizumab, nivolumab, cemiplimab, sintilimab, tislelizumab, atezolizumab (MPDL3280A), camrelizumab and toripalimab. In other embodiments, the PD-L1 antagonist is an anti-PD-L1 antibody independently selected from atezolizumab, durvalumab and avelumab.

In one embodiments, the PD-1 antagonist is pembrolizumab. In particular sub-embodiments, the method comprises administering 200 mg of pembrolizumab to the patient about every three weeks. In other sub-embodiments, the method comprises administering 400 mg of pembrolizumab to the patient about every six weeks.

In further sub-embodiments, the method comprises administering 2 mg/kg of pembrolizumab to the patient about every three weeks. In particular sub-embodiments, the patient is a pediatric patient.

In some embodiments, the PD-1 antagonist is nivolumab. In particular sub-embodiments, the method comprises administering 240 mg of nivolumab to the patient about every two weeks.

In other sub-embodiments, the method comprises administering 480 mg of nivolumab to the patient about every four weeks.

In some embodiments, the PD-1 antagonist is cemiplimab. In particular embodiments, the method comprises administering 350 mg of cemiplimab to the patient about every 3 weeks.

In some embodiments, the PD-1 antagonist is atezolizumab. In particular sub-embodiments, the method comprises administering 1200 mg of atezolizumab to the patient about every three weeks.

In some embodiments, the PD-1 antagonist is durvalumab. In particular sub-embodiments, the method comprises administering 10 mg/kg of durvalumab to the patient about every two weeks.

In some embodiments, the PD-1 antagonist is avelumab. In particular sub-embodiments, the method comprises administering 800 mg of avelumab to the patient about every two weeks.

When the Substituted Benzothiophene Derivatives are administered in combination with an anti-human PD-1 antibody (or antigen-binding fragment thereof), the anti-human PD-1 antibody (or antigen-binding fragment thereof) may be administered either simultaneously with, or before or after, the Substituted Benzothiophene Derivative. Either of the anti-human PD-1 antibody (or antigen-binding fragment thereof), and/or Substituted Benzothiophene Derivative of the present disclosure, or a pharmaceutically acceptable salt thereof, may be administered separately, by the same or different route of administration, or together in the same pharmaceutical composition as the other agent(s). The weight ratio of the anti-human PD-1 antibody (or antigen-binding fragment thereof) to Substituted Benzothiophene Derivative of the present disclosure, may be varied and will depend upon the therapeutically effective dose of each agent. Generally, a therapeutically effective dose of each will be used. Combinations including at least one anti-human PD-1 antibody (or antigen-binding fragment thereof), a Substituted Benzothiophene Derivative of the present disclosure, and optionally other active agents will generally include a therapeutically effective dose of each active agent. In such combinations, the anti-human PD-1 antibody (or antigen-binding fragment thereof), the Substituted Benzothiophene Derivative, and other active agents may be administered separately or in conjunction. In addition, the administration of one element may be prior to, concurrent with, or subsequent to the administration of other agent(s).

In one embodiment, this disclosure provides an anti-human PD-1 antibody (or antigen-binding fragment thereof), and/or Substituted Benzothiophene Derivative, and at least one other active agent as a combined preparation for simultaneous, separate or sequential use in treating cancer.

The disclosure also provides the use of a Substituted Benzothiophene Derivative of the present disclosure, for treating cancer, where the patient has previously (e.g., within 24-hours) been treated with an anti-human PD-1 antibody (or antigen-binding fragment thereof). The disclosure also provides the use of an anti-human PD-1 antibody (or antigen-binding fragment thereof) for treating a cellular proliferative disorder, where the patient has previously (e.g., within 24-hours) been treated with a Substituted Benzothiophene Derivative of the present disclosure.

The present disclosure further relates to methods of treating cancer, said method comprising administering to a subject in need thereof a combination therapy that comprises (a) a Substituted Benzothiophene Derivative of the present disclosure, and (b) an anti-human PD-1 antibody (or antigen-binding fragment thereof); wherein the anti-human PD-1 antibody (or antigen-binding fragment thereof) is administered once every 21 days.

Additionally, the present disclosure relates to methods of treating cancer, said method comprising administering to a subject in need thereof a combination therapy that comprises: (a) a Substituted Benzothiophene Derivative of the present disclosure, and (b) an anti-human PD-1 antibody (or antigen-binding fragment thereof. In specific embodiments, the cancer occurs as one or more solid tumors or lymphomas. In further specific embodiments, the cancer is selected from the group consisting of advanced or metastatic solid tumors and lymphomas. In still further specific embodiments, the cancer is selected from the group consisting of malignant melanoma, head and neck squamous cell carcinoma, MSI-H cancer, MMR deficient cancer, non-small cell lung cancer, urothelial carcinoma, gastric or gastroesophageal junction adenocarcinoma, breast adenocarcinoma, and lymphomas. In additional embodiments, the lymphoma is selected from the group consisting of diffuse large B-cell lymphoma, follicular lymphoma, mantle cell lymphoma, small lymphocytic lymphoma, mediastinal large B-cell lymphoma, splenic marginal zone B-cell lymphoma, extranodal marginal zone B-cell lymphoma of mucosa-associated lymphoid tissue (malt), nodal marginal zone B-cell lymphoma, lymphoplasmacytic lymphoma, primary effusion lymphoma, Burkitt lymphoma, anaplastic large cell lymphoma (primary cutaneous type), anaplastic large cell lymphoma (systemic type), peripheral T-cell lymphoma, angioimmunoblastic T-cell lymphoma, adult T-cell lymphoma/leukemia, nasal type extranodal NK/T-cell lymphoma, enteropathy-associated T-cell lymphoma, gamma/delta hepatosplenic T-cell lymphoma, subcutaneous panniculitis-like T-cell lymphoma, mycosis fungoides, and Hodgkin lymphoma. In particular embodiments, the cellular proliferative disorder is a cancer that has metastasized, for example, a liver metastases from colorectal cancer. In additional embodiments, the cellular proliferative disorder is a cancer is classified as stage III cancer or stage IV cancer. In instances of these embodiments, the cancer is not surgically respectable.

In embodiments of the methods disclosed herein, the anti-human PD-1 antibody (or antigen binding fragment thereof) is administered by intravenous infusion or subcutaneous injection.

In one embodiment, the present disclosure provides compositions comprising a Substituted Benzothiophene Derivative, a pharmaceutically acceptable carrier, and an anti-human PD-1 antibody (or antigen-binding fragment thereof).

In another embodiment, the present disclosure provides compositions comprising a Substituted Benzothiophene Derivative, a pharmaceutically acceptable carrier, and pembrolizumab.

In one embodiment, the present disclosure provides compositions comprising a Substituted Benzothiophene Derivative, a pharmaceutically acceptable carrier, and two additional therapeutic agents, one of which is an anti-human PD-1 antibody (or antigen-binding fragment thereof), and the other of which is independently selected from the group consisting of anticancer agents.

A compound of the present disclosure may be employed in conjunction with anti-emetic agents to treat nausea or emesis, including acute, delayed, late-phase, and anticipatory emesis, which may result from the use of a compound of the present disclosure, alone or with radiation therapy. For the prevention or treatment of emesis, a compound of the present disclosure may be used in conjunction with other anti-emetic agents, especially neurokinin-1 receptor antagonists, 5HT3 receptor antagonists, such as ondansetron, granisetron, tropisetron, and zatisetron, GABAB receptor agonists, such as baclofen, a corticosteroid such as Decadron (dexamethasone), Kenalog, Aristocort, Nasalide, Preferid, Benecorten or others such as disclosed in U.S. Pat. Nos. 2,789,118, 2,990,401, 3,048,581, 3,126,375, 3,929,768, 3,996,359, 3,928,326 and 3,749,712, an antidopaminergic, such as the phenothiazines (for example prochlorperazine, fluphenazine, thioridazine and mesoridazine), metoclopramide or dronabinol. In another example, conjunctive therapy with an anti-emesis agent selected from a neurokinin-1 receptor antagonist, a 5HT3 receptor antagonist and a corticosteroid is disclosed for the treatment or prevention of emesis that may result upon administration of the Substituted Benzothiophene Derivatives.

A Substituted Benzothiophene Derivative may also be administered with an agent useful in the treatment of anemia. Such an anemia treatment agent is, for example, a continuous erythropoiesis receptor activator (such as epoetin alfa).

A Substituted Benzothiophene Derivative may also be administered with an agent useful in the treatment of neutropenia. Such a neutropenia treatment agent is, for example, a hematopoietic growth factor which regulates the production and function of neutrophils such as a human granulocyte colony stimulating factor, (G-CSF). Examples of a G-CSF include filgrastim.

The Substituted Benzothiophene Derivatives may be useful when co-administered with other treatment modalities, including but not limited to, radiation therapy, surgery, and gene therapy. Accordingly, in one embodiment, the methods of treating cancer described herein, unless stated otherwise, can optionally include the administration of an effective amount of radiation therapy. For radiation therapy, y-radiation is preferred.

The methods of treating cancers described herein can optionally include the administration of an effective amount of radiation (i.e., the methods of treating cancers described herein optionally include the administration of radiation therapy).

The methods of treating cancer described herein include methods of treating cancer that comprise administering a therapeutically effective amount of a Substituted Benzothiophene Derivative in combination with radiation therapy and/or in combination with a second compound selected from: an estrogen receptor modulator, an androgen receptor modulator, a retinoid receptor modulator, a cytotoxic/cytostatic agent, an antiproliferative agent, a prenyl-protein transferase inhibitor, an HMG-CoA reductase inhibitor, an HIV protease inhibitor, a reverse transcriptase inhibitor, an angiogenesis inhibitor, PPAR-γ agonists, PPAR-δ agonists, an inhibitor of inherent multidrug resistance, an anti-emetic agent, an agent useful in the treatment of anemia, an agent useful in the treatment of neutropenia, an immunologic-enhancing drug, an inhibitor of cell proliferation and survival signaling, a bisphosphonate, an aromatase inhibitor, an siRNA therapeutic, γ-secretase and/or NOTCH inhibitors, agents that interfere with receptor tyrosine kinases (RTKs), an agent that interferes with a cell cycle checkpoint, and any of the additional therapeutic agents listed herein.

Additional embodiments of the disclosure include the pharmaceutical compositions, combinations, uses and methods set forth in above, wherein it is to be understood that each embodiment may be combined with one or more other embodiments, to the extent that such a combination is consistent with the description of the embodiments. It is further to be understood that the embodiments provided above are understood to include all embodiments, including such embodiments as result from combinations of embodiments.

### Kits

In one aspect, provided is a kit comprising a therapeutically effective amount of a Substituted Benzothiophene Derivative, or a pharmaceutically acceptable salt, solvate or ester of said compound and a pharmaceutically acceptable carrier, vehicle or diluent.

In another aspect provided is a kit comprising an amount of a Substituted Benzothiophene Derivative, and an amount of at least one additional therapeutic agent listed above, wherein the amounts of the two or more active ingredients result in a desired therapeutic effect. In one embodiment, the Substituted Benzothiophene Derivative, and the one or more additional therapeutic agents are provided in the same container. In one embodiment, the Substituted Benzothiophene Derivative, and the one or more additional therapeutic agents are provided in separate containers.

1. A compound of formula (I): 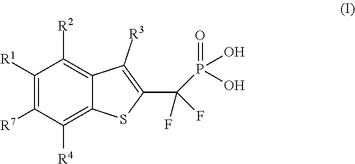
or a pharmaceutically acceptable salt thereof,
wherein:
R¹ is selected from H, C₁-C₁₀ alkyl, C₂-C₁₀ alkynyl, halo, —OH, —C(O)OH, —CN, —S(O)₂R⁹, —C(O)N(R⁵)(R⁶), —NHC(O)N(R⁵)(R⁶), —(C₁-C₁₀ alkenyl)ₙ-NHC(O)—(C₁-C₁₀ alkyl), —(C₁-C₁₀ alkenyl)ₙ-S(O)(NH)R⁹, NHC(O)—(C₁-C₁₀ alkenyl)ₙ-(5 or 6-membered monocyclic heteroaryl), —NH₂, —NHC(O)—CF₃, —(C₁-C₁₀ alkenyl)ₙ-NHC(O)—(C₃-C₇ monocyclic cycloalkyl), —O—(C₁-C₁₀ alkyl), —C(═NH)(NH₂), —O—(C₁-C₁₀ alkylene)-(C₆-C₁₀ aryl), —O—(C₆-C₁₀ aryl), —O-(5 or 6-membered monocyclic heteroaryl), —O-(8 to 10-membered bicyclic heteroaryl), 5 or 6-membered monocyclic heteroaryl, 4 to 6-membered monocyclic heterocycloalkenyl, —(8 to 10-membered bicyclic heteroaryl), —CH₂-(3 to 7-membered monocyclic heterocycloalkyl), 9 or 10-membered bicyclic heterocycloalkyl, —CH(NH₂)(CF₃), and —CH(NH)-phenyl, wherein said phenyl group, said C₆-C₁₀ aryl group, said C₃-C₇ monocyclic cycloalkyl group, said 5 or 6-membered monocyclic heteroaryl group, said 4 to 6-membered heterocycloalkenyl group, said 3 to 7-membered monocyclic heterocycloalkyl group, and said 9 or 10-membered monocyclic heterocycloalkyl group may be optionally substituted with 1-3 groups, which can be the same or different, and which are selected from C₁-C₁₀ alkyl, C₁-C₁₀ haloalkyl, —O—(C₁-C₁₀ alkyl), C₃-C₇ monocyclic cycloalkyl, and halo; and wherein said C₃-C₇ monocyclic cycloalkyl group, said 4 to 6-membered heterocycloalkenyl group, and said 3 to 7-membered monocyclic heterocycloalkyl group can have one or more ring carbon atoms or ring heteroatoms optionally substituted with an oxo group; and wherein any C₁-C₁₀ alkyl group can be optionally substituted with 1-3 groups, which can be the same or different, and which are selected from —OH, halo, —CN, —OR⁹, —N(R⁹)₂, —SO₂(R⁹), —S(O)₂N(R⁹)₂, —S(O)NH(R⁹), halo, —NHC(O)R⁹, and —C(O)N(R⁹)₂;
R² is H or halo;
R³ is selected from H, halo, —CN, C₁-C₁₀ alkyl, C₃-C₇ monocyclic cycloalkyl, and C₁-C₁₀ haloalkyl;
R⁴ is selected from H, halo, —OH, —CN, —(C₁-C₁₀ alkylene)ₙ-(5 or 6-membered monocyclic heteroaryl), —(C₁-C₁₀ alkylene)-(C₆-C₁₀ aryl), —(C₁-C₁₀ alkylene)-S-(5 or 6-membered monocyclic heteroaryl), —(C₁-C₁₀ alkylene)-Si(C₁-C₆ alkyl)₃, —O—(C₁-C₁₀ alkyl), —S—(C₁-C₁₀ alkyl), —O—(C₁-C₁₀ alkenylene), —O—(C₁-C₁₀ haloalkyl), —O—(C₁-C₁₀ hydroxyalkyl), —O—(C₁-C₁₀ alkylene)-S(O)₂R⁹, —O—(C₁-C₁₀ alkylene)-CN, —O—(C₁-C₁₀ alkylene)-O—(C₁-C₁₀ alkyl), —O—(C₁-C₁₀ alkylene)-O—(C₁-C₁₀ haloalkyl), —O—(C₁-C₁₀ alkylene)-(C₆-C₁₀ aryl), —O—(C₁-C₁₀ alkylene)-(C₃-C₇ monocyclic cycloalkyl), —O—(C₁-C₁₀ alkylene)-(3 to 7-membered monocyclic heterocycloalkyl), O—(C₁-C₁₀ alkylene)-(5 to 10-membered bridged heterocycloalkyl), —O—(C₁-C₁₀ alkylene)-(5 or 6-membered monocyclic heteroaryl), —C₁-C₁₀ alkenyl, —(C₁-C₁₀ alkylene)-O—(C₁-C₁₀ alkyl), —O—(C₁-C₁₀ alkylene)-S(O)₂N(R⁹)₂, —O—(C₁-C₁₀ alkylene)-NHS(O)₂—(C₁-C₁₀ alkyl), —O—(C₁-C₁₀ alkylene)-NHS(O)₂—(C₁-C₁₀ haloalkyl), —O—(C₁-C₁₀ alkylene)-NHS(O)₂—(C₃-C₇ monocyclic cycloalkyl), —O—(C₁-C₁₀ alkylene)-(C₅-C₁₀ bicyclic cycloalkyl), —O—(C₁-C₁₀ alkylene)-C(O)NH-phenyl, —O—(C₁-C₁₀ alkylene)-C(O)NH₂, —O—(C₁-C₁₀ alkylene)-C(O)NH—(5 or 6-membered monocyclic heteroaryl), —O—(C₁-C₁₀ alkylene)-C(O)NH—(C₃-C₇ monocyclic cycloalkyl), —O—(C₁-C₁₀ alkylene)-C(O)NH—(C₁-C₁₀ alkyl), —O—(C₁-C₁₀ alkylene)-C(O)NH—(C₁-C₁₀ haloalkyl), —(C₁-C₁₀ alkylene)ₙ-(3 to 7-membered monocyclic heterocycloalkyl), —(C₁-C₁₀ alkylene)ₙ-(8 to 10-membered bicyclic heteroaryl), —(C₁-C₁₀ alkylene)ₙ-(9 to 14-membered tricyclic heteroaryl), —(C₁-C₁₀ alkylene)ₙ-(10 to 14-membered tricyclic heterocycloalkyl), —O—(C₁-C₁₀ alkylene)ₙ-(3 to 7-membered monocyclic heterocycloalkyl), —O—(C₁-C₁₀ alkylene)ₙ-(C₅-C₁₀ membered bicyclic cycloalkyl), —O—(C₁-C₁₀ alkylene)ₙ-(10 to 14-membered tricyclic heterocycloalkyl), —NH—(C₁-C₁₀ alkyl), —NH—(C₁-C₁₀ haloalkyl), —NH—(C₁-C₁₀ hydroxyalkyl), —NH—(C₁-C₁₀ alkylene)-CN, —NH—(C₁-C₁₀ alkylene)-O—(C₁-C₁₀ alkyl), —NH—(C₁-C₁₀ alkylene)-O—(C₁-C₁₀ haloalkyl), —NH—(C₁-C₁₀ alkylene)-(C₆-C₁₀ aryl), —NH—(C₁-C₁₀ alkylene)-(C₃-C₇ monocyclic cycloalkyl), —NH—(C₁-C₁₀ alkylene)-(3 to 7-membered monocyclic heterocycloalkyl), —NH—(C₁-C₁₀ alkylene)-(5 or 6-membered monocyclic heteroaryl), —NH—(C₁-C₁₀ haloalkyl), —NH—(C₁-C₁₀ hydroxyalkyl), —NH—(C₁-C₁₀ alkylene)-S(O)₂—(C₁-C₁₀ alkyl), —NH—(C₁-C₁₀ alkylene)-S(O)₂N(R⁹)₂, —NH—(C₁-C₁₀ alkylene)-NHS(O)₂—(C₁-C₁₀ alkyl), —NH—(C₁-C₁₀ alkylene)-NHS(O)₂—(C₁-C₁₀ haloalkyl), —NH—(C₁-C₁₀ alkylene)-NHS(O)₂—(C₃-C₇ monocyclic cycloalkyl), —NH—(C₁-C₁₀ alkylene)-(C₆-C₁₀ bicyclic cycloalkyl), —NH—(C₁-C₁₀ alkylene)-C(O)NH-phenyl, —NH—(C₁-C₁₀ alkylene)-C(O)NH—(C₃-C₇ monocyclic cycloalkyl), —NH—(C₁-C₁₀ alkylene)-C(O)NH—(C₁-C₁₀ alkyl), —NH—(C₁-C₁₀ alkylene)-C(O)NH—(C₁-C₁₀ haloalkyl), —NH—(C₁-C₁₀ alkylene)ₙ-(3 to 7-membered monocyclic heterocycloalkyl), —NH—(C₁-C₁₀ alkylene)ₙ-(6 to 10 membered bicyclic cycloalkyl), —(C₁-C₁₀ alkylene)ₙ-S(0)₂N(R⁹)₂, and —NH—(C₁-C₁₀ alkylene)ₙ-(10 to 14-membered tricyclic heterocycloalkyl), wherein said 5 or 6-membered monocyclic heteroaryl group, said 3 to 7-membered monocyclic heterocycloalkyl group, said C₆-C₁₀ aryl group, said 8 to 10-membered bicyclic heteroaryl group, said 6 to 10-membered bicyclic heterocycloalkyl group, said 10 to 14-membered membered tricyclic heterocycloalkyl group, said C₃-C₇ monocyclic cycloalkyl group, said 5 to 10 membered bridged cycloalkyl group, 5 to 10-membered bridged heterocycloalkyl group and C₆-C₁₀ bicyclic cycloalkyl group may be optionally substituted with one or more R^{A} groups, which can be the same or different; and wherein the C₁-C₁₀ alkyl moiety of a —O—(C₁-C₁₀ alkyl) group can be optionally substituted with 1-3 groups, which can be the same or different, and which are selected from —OH, halo, —CN, —OR⁹, —N(R⁹)₂, —SO₂(R⁹), —S(O)₂N(R⁹)₂, —S(O)NH(R⁹), halo, —NHC(O)R⁹, and —C(O)N(R⁹)₂;
R⁵ is selected from H, C₁-C₁₀ alkyl, and —(C₁-C₁₀ alkylene)-O—(C₁-C₁₀ alkyl);
R⁶ is selected from H, C₁-C₁₀ alkyl, —OR⁹, —(C₁-C₁₀ alkylene)-S—(C₁-C₁₀ haloalkyl), —(C₁-C₁₀ alkylene)ₙ-OR⁹, —(C₁-C₁₀ alkylene)ₙ-(C₆-C₁₀ aryl), —(C₁-C₁₀ alkylene)ₙ-CN, —(C₁-C₁₀ alkylene)ₙ-(5 or 6-membered monocyclic heteroaryl), —(C₁-C₁₀ alkylene)ₙ-(8 to 10-membered bicyclic heteroaryl), —(C₁-C₁₀ alkylene)ₙ-(3 to 7-membered monocyclic cycloalkyl), —(C₁-C₁₀ alkylene)ₙ-(3 to 7-membered monocyclic heterocycloalkyl), —(C₁-C₁₀ alkylene)ₙ-(6 to 10 membered bicyclic heterocycloalkyl), and —(C₁-C₁₀ alkylene)ₙ-(10 to 14-membered membered tricyclic heterocycloalkyl) group, wherein said C₆-C₁₀ aryl group, said 5 or 6-membered monocyclic heteroaryl group, said 8 to 10-membered bicyclic heteroaryl group, said 10 to 14-membered membered tricyclic heterocycloalkyl group, said C₃-C₇ monocyclic cycloalkyl group, said 3 to 7-membered monocyclic heterocycloalkyl group, and said 6 to 10 membered bicyclic heterocycloalkyl group can each be optionally substituted with one or more R^{B} groups, which can be the same or different, and wherein the C₁-C₁₀ alkyl group or C₁-C₁₀ alkylenyl group can be optionally substituted with 1-3 groups, which can be the same or different, and which are selected from —OH, halo, —CN, —OR⁹, —N(R⁹)₂, —SO₂(R⁹), —S(O)₂N(R⁹)₂, —S(O)NH(R⁹), halo, phenyl, —NHC(O)R⁹, C₁-C₁₀ haloalkyl, and —C(O)N(R⁹)₂;
R⁷ is selected from H, halo, CN, —C(O)N(R′)₂, —O—(C₁-C₁₀ hydroxyalkyl), and —NHC(O)R⁸;
each occurrence of R⁸ is independently selected from H, C₁-C₁₀ alkyl, phenyl, and 5 or 6-membered monocyclic heteroaryl;
each occurrence of R⁹ is independently selected from H, C₁-C₁₀ alkyl, C₁-C₁₀ haloalkyl, —SO₂CH₃, C₃-C₇ monocyclic cycloalkyl, 3 to 7-membered monocyclic heterocycloalkyl, C₆-C₁₀ aryl, —(C₁-C₁₀ alkylene)ₙ-(C₆-C₁₀ aryl), and 5 or 6-membered monocyclic heteroaryl;
each occurrence of R^{A} is independently selected from C₁-C₁₀ alkyl, halo, —CN, C₁-C₁₀ haloalkyl, —O—(C₁-C₁₀ haloalkyl), oxo, —O—(C₁-C₁₀ alkyl), —C₃-C₇ monocyclic cycloalkyl, 5 or 6-membered monocyclic heteroaryl, C₆-C₁₀ aryl, —C(O)-(3 to 7-membered monocyclic heterocycloalkyl), —S(O)₂—(C₁-C₁₀ alkyl), and —O-(3 to 7-membered monocyclic heterocycloalkyl); wherein said C₃-C₇ monocyclic cycloalkyl group and said 3 to 7-membered monocyclic heterocycloalkyl group can be further substituted with C₁-C₁₀ alkyl, halo, C₁-C₁₀ haloalkyl or —CN;
each occurrence of R^{B} is independently selected from C₁-C₁₀ alkyl, halo, —OH, oxo, —SO2NH2, C₁-C₁₀ haloalkyl, C₁-C₁₀ hydroxyalkyl, —O—(C₁-C₁₀ alkyl), —O—(C₁-C₁₀ alkylene)-O—(C₁-C₁₀ alkyl), —O—(C₁-C₁₀ haloalkyl), —CN, —C(O)—(C₁-C₁₀ alkyl), —C(O)—(C₃-C₇ monocyclic cycloalkyl), —C(O)-(3 to 7-membered monocyclic heterocycloalkyl), —O-(3 to 7-membered monocyclic heterocycloalkyl), —S(O)₂—(C₁-C₁₀ alkyl), phenyl, benzyl, —O-phenyl, —O-benzyl, —S— phenyl, C₃-C₇ monocyclic cycloalkyl, —O—(C₁-C₁₀ alkylene)-phenyl, —O—(C₁-C₁₀ alkylene)ₙ-(3 to 7-membered monocyclic heterocycloalkyl), —(C₁-C₁₀ alkylene)ₙ-(5 or 6-membered monocyclic heteroaryl); wherein said phenyl group, said C₃-C₇ monocyclic cycloalkyl group, said 3 to 7-membered monocyclic heterocycloalkyl group, and said 5 or 6-membered monocyclic heteroaryl group, can be optionally substituted with 1-3 groups, which can be the same or different, and are selected from C₁-C₁₀ alkyl, —O—(C₁-C₁₀ alkyl), halo, C₁-C₁₀ haloalkyl, CN, and —OH; and
each occurrence of n is independently 0 or 1.

2. The compound of claim 1, or a pharmaceutically acceptable salt thereof, wherein R² is H.

3. The compound of claim 1, or a pharmaceutically acceptable salt thereof, wherein R³ is selected from H, Br, Cl, —CN—CH₃, —CHF₂, cyclopropyl, and CF₃.

4. The compound of claim 1, or a pharmaceutically acceptable salt thereof, wherein R¹ is —C(O)N(R⁵)(R⁶) or 5 or 6-membered monocyclic heteroaryl.

5. The compound of claim 4, or a pharmaceutically acceptable salt thereof, wherein R¹ is —C(O)NH(R⁶).

6. The compound of claim 4, or a pharmaceutically acceptable salt thereof, wherein R¹ is 5-membered monocyclic heteroaryl.

7. The compound of claim 1, wherein R⁴ is selected from H, halo, —O—(C₁-C₁₀ haloalkyl), —O—(C₁-C₁₀ hydroxyalkyl), —O—(C₁-C₁₀ alkylene)-CN, —O—(C₁-C₁₀ alkylene)-S(O)₂—(C₁-C₁₀ alkyl), —O—(C₁-C₁₀ alkylene)-S(O)₂NH—(C₁-C₁₀ alkyl), —O—(C₁-C₁₀ alkylene)-(C₆-C₁₀ aryl), —O—(C₁-C₁₀ alkylene)-(C₃-C₇ monocyclic cycloalkyl), —O—(C₁-C₁₀ alkylene)-(3 to 7-membered monocyclic heterocycloalkyl), —O—(C₁-C₁₀ alkylene)-(5 or 6-membered monocyclic heteroaryl), and —O—(C₁-C₁₀ alkylene)ₙ-(3 to 7-membered monocyclic heterocycloalkyl), wherein said C₃-C₇ monocyclic cycloalkyl can be optionally substituted with 1-3 groups, each independently selected from halo and —CN.

8. The compound of claim 1 of formula (Ia): 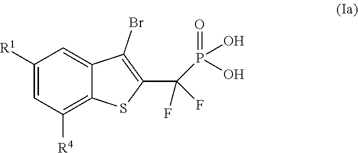
or a pharmaceutically acceptable salt thereof,
wherein:
R¹ is —C(O)NH(R⁶) or 5-membered monocyclic heteroaryl,
R⁴ is selected from R⁴ is selected from H, halo, —O—(C₁-C₁₀ haloalkyl), —O—(C₁-C₁₀ hydroxyalkyl), —O—(C₁-C₁₀ alkylene)-CN, —O—(C₁-C₁₀ alkylene)-S(O)₂—(C₁-C₁₀ alkyl), —O—(C₁-C₁₀ alkylene)-S(O)₂NH—(C₁-C₁₀ alkyl), —O—(C₁-C₁₀ alkylene)-(C₆-C₁₀ aryl), —O—(C₁-C₁₀ alkylene)-(C₃-C₇ monocyclic cycloalkyl), —O—(C₁-C₁₀ alkylene)-(3 to 7-membered monocyclic heterocycloalkyl), —O—(C₁-C₁₀ alkylene)-(5 or 6-membered monocyclic heteroaryl), and —O—(C₁-C₁₀ alkylene)ₙ-(3 to 7-membered monocyclic heterocycloalkyl), wherein said C₃-C₇ monocyclic cycloalkyl can be optionally substituted with 1-3 groups, each independently selected from halo and —CN;
R⁶ is selected from H, C₁-C₁₀ alkyl, and —(C₁-C₁₀ alkylene)ₙ-5 or 6-membered monocyclic heteroaryl; and
each occurrence of n is independently 0 or 1.

9. The compound of claim 1, or a pharmaceutically acceptable salt thereof, wherein R¹ is selected from: H, I, —OH, —C(O)NH₂, —C(O)NHCH₃, —C—CH, —C(O)NHCH₂CH₃, —C(O)NHCD₃-C(O)OH, —CH₂OH, —CH₂CN, pyrazolyl, —CH₂S(O)(NH)—CH₃, —S(O)(NH)—CH₃, imidazolyl, —C(O)NHCH₂CN, —C(O)NHCH₂CH₂CN, —S(O)₂NHCH₃, —CH₂NHC(O)CH₃,

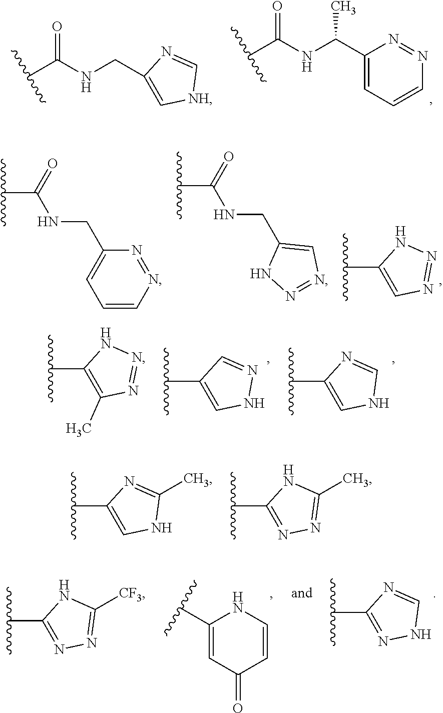

10. The compound of claim 1, or a pharmaceutically acceptable salt thereof, wherein R⁴ is selected from: H, —O(CH₂)₃CF₃, —O(CH₂)₂C(CH₃)₂OH, —O(CH₂)₃SO₂CH₃, —O(CH₂)₃SO₂NH₂, —O(CH₂)₄C(O)NH₂, —OCH(CH₃)CH₂CH₂CH₃, —O(CH₂)₃SO₂N(CH₃)₂, —O(CH₂)₃SO₂NHCH₃, —O(CH₂)₄SO₂CH₃, —(CH₂)₄SO₂NH₂, —NH(CH₂)SO₂NH₂, —O(CH₂)₄C(O)NH₂, —O(CH₂)₂NHSO₂CH₃, —O(CH₂)₂CF₂CH₃, —O—CH(CH₃)CH₂CH₂CH₃, pyrazolyl, —S(CH₂)₃CH₃, —CH₂CH₂-phenyl, —C≡C(CH₂)₂SO₂NH₂, —CH₂CH₂—Si(CH₃)₃, —O(CH₂)₃SO₂CF₃, —OCH(CH₂OH)CH₂CH₂CH₃, —OCH₂CH(F)CH₂CF₃,

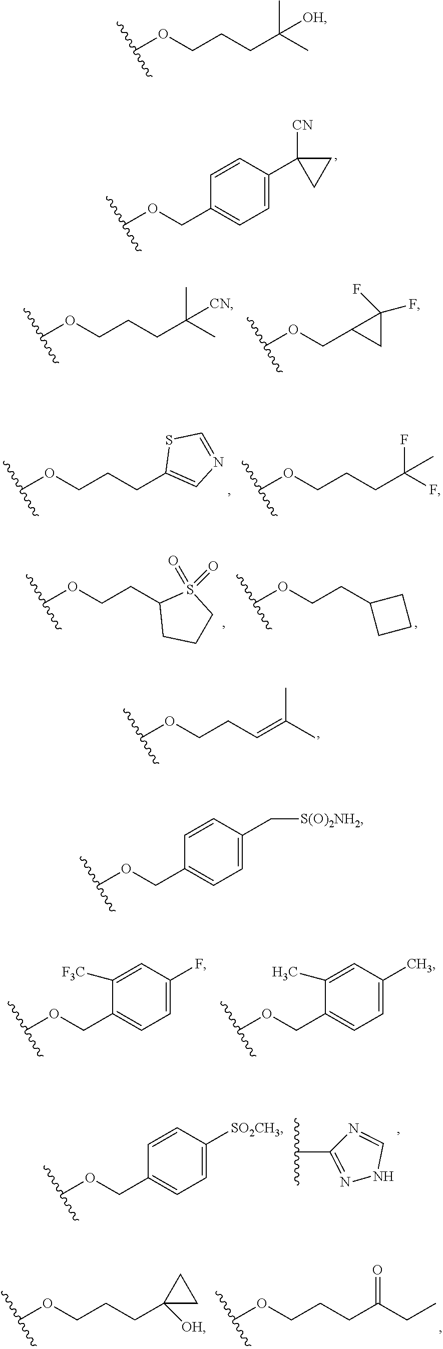

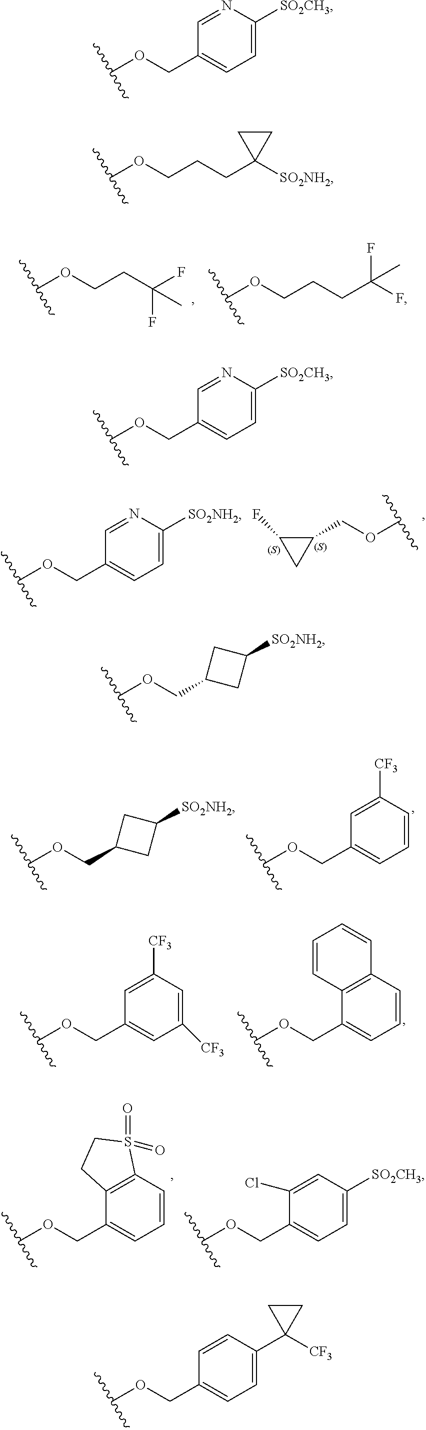

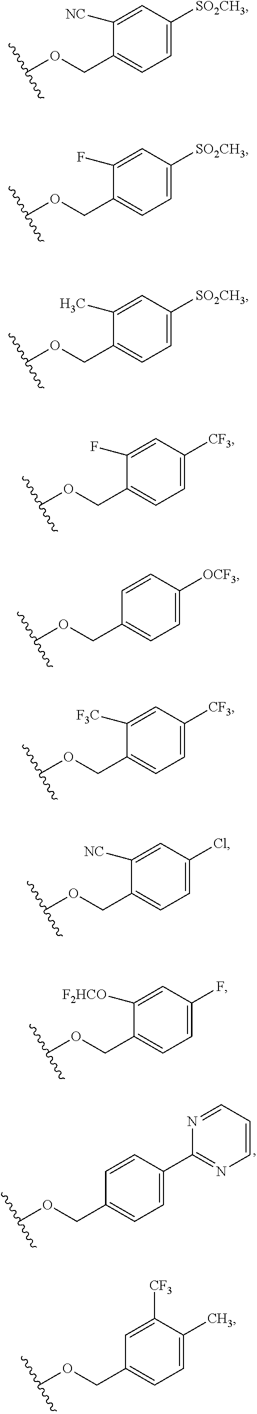

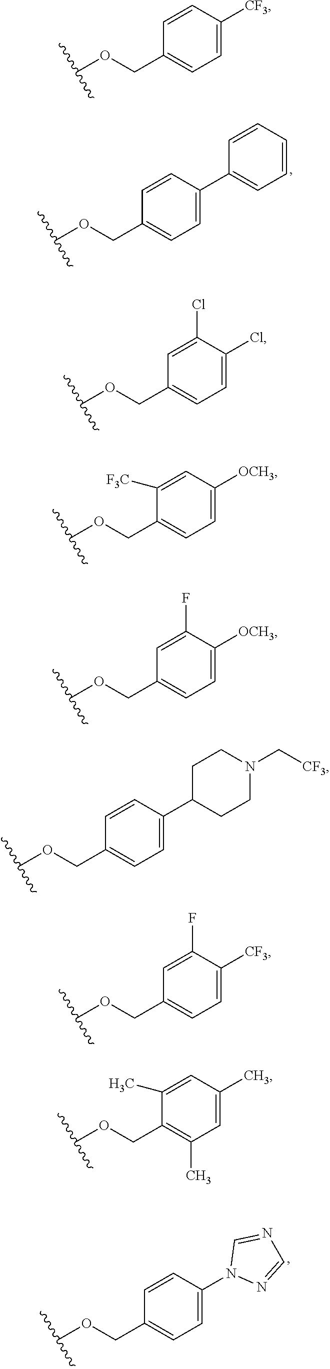

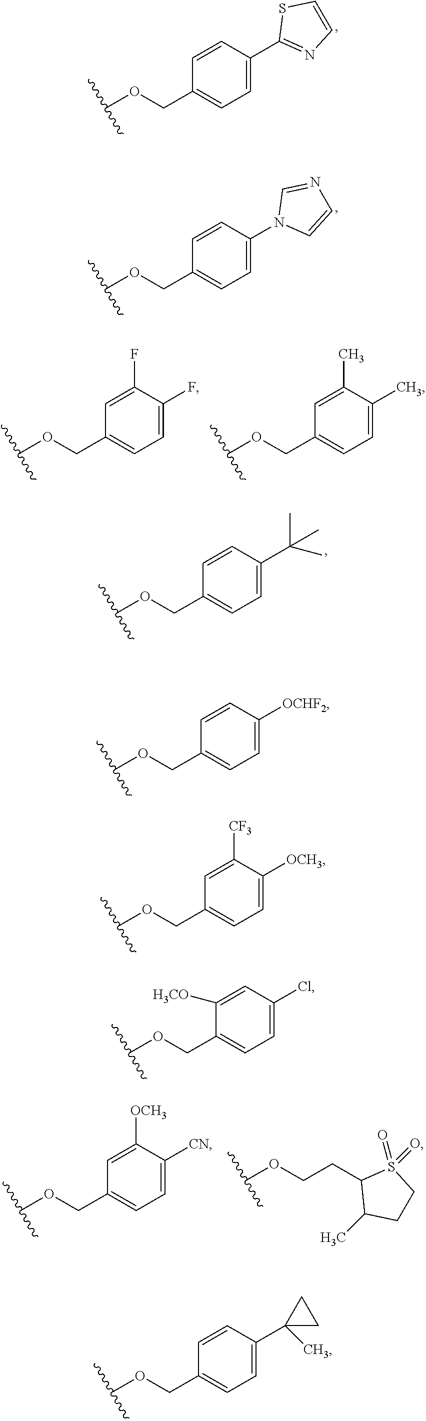

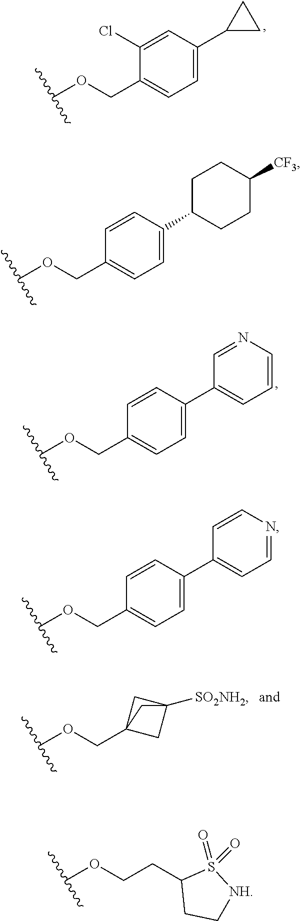 [SMILES: *OCc1ccc(C2CC2)cc1Cl ; *OCc1ccc([C@H]2CC[C@H](C(F)(F)F)CC2)cc1 ; *OCc1ccc(-c2cccnc2)cc1 ; *OCc1ccc(-c2ccncc2)cc1 ; *OCC12CC(S(N)(=O)=O)(C1)C2 ; *OCCC1CCNS1(=O)=O]

11. A compound selected from any of the compounds numbered 1-464 in the above specification, or a pharmaceutically acceptable salt thereof.

12. A pharmaceutical composition comprising an effective amount of the compound of claim 1, or a pharmaceutically acceptable salt thereof, and a pharmaceutically acceptable carrier.

13. The pharmaceutical composition of claim 12 further comprising one or more additional therapeutic agents, wherein said additional therapeutic agents are selected from anticancer agents.

14. A method of treating cancer in a patient in need thereof, comprising administering to the patient an effective amount of the compound of claim 1, or a pharmaceutically acceptable salt thereof.

15. The method of claim 14, further comprising administering one or more additional therapeutic agents, wherein said additional therapeutic agents are selected from anticancer agents.

16. The pharmaceutical composition of claim 13, wherein said additional therapeutic agents comprise pembrolizumab.

17. The method of claim 15, wherein said additional therapeutic agents comprise pembrolizumab.
